SEQUENCE LISTING (2 sequences)

SEQ ID NO: 1 - protein, 31 aa, Homo sapiens
  mat_peptide (1)..(31)
HAEGTFTSDVSSYLEGQAAKEFIAWLVKGRG

SEQ ID NO: 2 - protein, 39 aa, Heloderma suspectum
  mat_peptide (1)..(39)
HGEGTFTSDLSKQMEEEAVRLFIEWLKNGGPSSGAPPPS

(19) United States
(12) Patent Application Publication
(10) Pub. No.: US 2013/0012432 A1
(43) Pub. Date: Jan. 10, 2013

(54) Peptides for Treatment of Obesity

(75) Inventors: Kilian Waldemar Conde-Frieboes, Maalov (DK); Jane Spetzler, Broenshoej (DK); Ulrich Sensfuss, Koebenhavn V (DK); Birgitte Schjellerup Wulff, Virum (DK); Henning Thoegersen, Farum (DK); Jens Christian Norrild, Birkeroed (DK)

(73) Assignee: Novo Nordisk A/S, Bagsvaerd (DK)

(21) Appl. No.: 13/580,042

(22) PCT Filed: Feb. 28, 2011
(86) PCT No.: PCT/EP2011/052909

§ 371 Date: Sep. 5, 2012

Related U.S. Application Data
(60) Provisional application No. 61/310,494, filed on Mar. 4, 2010.

(30) Foreign Application Priority Data
Feb. 26, 2010 (EP) 10154818.8

(51) Int. Cl. A61K 38/12; A61P 3/10; A61P 3/04; A61P 3/00; A61P 35/00; A61P 15/00; A61P 9/12; A61P 1/16; A61P 19/08; A61P 19/02; A61P 29/00; A61P 37/06; C07K 7/56; A61P 9/00

(52) U.S. Cl. 514/4.9; 514/6.9; 514/4.8; 530/317; 530/323; 530/327; 514/21.1; 514/19.9; 514/16.4; 514/7.4; 514/16.8; 514/15.7; 514/17.7; 514/17.9

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates to novel peptides with improved solubility which are specific to one or more melanocortin receptors, to the use of said peptides in therapy, to methods of treatment comprising administration of said peptides to patients, and to the use of said peptides in the manufacture of medicaments.

### BACKGROUND OF THE INVENTION

Obesity is a well known risk factor for the development of common diseases such as atherosclerosis, hypertension, type 2 diabetes, dyslipidaemia, coronary heart disease, gallbladder disease, osteoarthritis, premature death, certain types of cancer and various other malignancies. It also causes considerable problems through reduced motility and decreased quality of life. In the industrialized western world the prevalence of obesity has increased significantly in the past few decades. Only a few pharmacological treatments are available to date, namely Sibutramine (Abbot, acting via serotonergic and noradrenaline mechanisms), Orlistat (Roche and GlaxoSmithKline, works by reducing fat uptake from the gut). Because obesity represents a very high risk factor in serious and even fatal common diseases, its treatment should be a high public health priority and there is a need for pharmaceutical compounds useful in the treatment of obesity.

Pro-opiomelanocortin (POMC) is the precursor of the melanocortin family of peptides, which include α-, β- and γ-melanocyte stimulating hormone (MSH) peptides and adrenocorticotropic hormone (ACTH), as well as other peptides such as β-endorphin. POMC is expressed in neurons of the central and peripheral nervous system and in the pituitary. Several of the melanocortin peptides, including ACTH and α-MSH, have been shown to have appetite-suppressing activity when administered to rats by intracerebroventricular (icy) injection [Vergoni et al, European Journal of Pharmacology 179, 347-355 (1990)]. An appetite-suppressing effect is also obtained with the artificial cyclic α-MSH analogue, MT-II.

Five melanocortin receptor subtypes, MC1-5 receptors have been identified. MC1, MC2 and MC5 receptors are mainly expressed in peripheral tissues, whereas MC3 and MC4 receptors are mainly centrally expressed. MC3 receptors are also expressed in several peripheral tissues. In addition to being involved in energy homeostasis, MC3 receptors have also been suggested to be involved in several inflammatory diseases. It has been suggested that MC5 receptors are involved in exocrine secretion and in inflammation. MC4 receptors have been shown to be involved in the regulation of body weight and feeding behavior, as MC4 knock-out mice develop obesity [Huzar et al., Cell 88, 131-141 (1997)] and common variants near MC4 receptor have been found to be associated with fat mass, weight and risk of obesity [Loos et al. Nat Genet., 40(6):768-75 (2008)]. Furthermore, studies with mice showed that overexpression in the mouse brain of the melanocortin receptor antagonists agouti protein and agouti-related protein (AGRP), led to the development of obesity [Kleibig et al., PNAS 92, 4728-4732 (1995)]. Moreover, icy injection of a C-terminal fragment of AGRP increases feeding and antagonizes the inhibitory effect of α-MSH on food intake.

MC4 receptor agonists could serve as anorectic drugs and/or energy expenditure increasing drugs and be useful in the treatment of obesity or obesity-related diseases, as well as in the treatment of other diseases, disorders or conditions which may be ameliorated by activation of MC4 receptor. On the other hand, MC4 receptor antagonists may be useful in the treatment of cachexia or anorexia, of waisting in frail elderly patients, chronic pain, neuropathy and neurogenic inflammation.

A large number of patent applications disclose various classes of non-peptidic small molecules as melanocortin receptor modulators, of which examples hereof are WO 03/009850, WO 03/007949 and WO 02/081443. The use of peptides as melanocortin receptor modulators is disclosed in a number of patent documents, e.g. WO 03/006620, U.S. Pat. No. 5,731,408 and WO 98/27113. Hadley [Pigment Cell Res. (1991) 4:180-185] reported a prolonged effect of specific melanotropic peptides conjugated to fatty acids, the prolongation effected by a transformation of the modulators from being reversibly acting to being irreversibly acting being caused by the conjugated fatty acids.

### SUMMARY OF THE INVENTION

The present invention relates to novel peptides which are specific to one or more melanocortin receptors with improved solubility at neutral to weakly basic pH, to the use of said peptides in therapy, to methods of treatment comprising administration of said peptides to patients, and to the use of said peptides in the manufacture of medicaments.

The present inventors have surprisingly found that specific peptide conjugates have a high modulating effect on one or more melanocortin receptors, i.e., the MC1, MC2, MC3, MC4 or MC5. Accordingly, in a first embodiment (embodiment 1), the invention relates to compounds (more particularly compounds acting as melanocortin receptor agonists or antagonists) according to formula I or formula II:

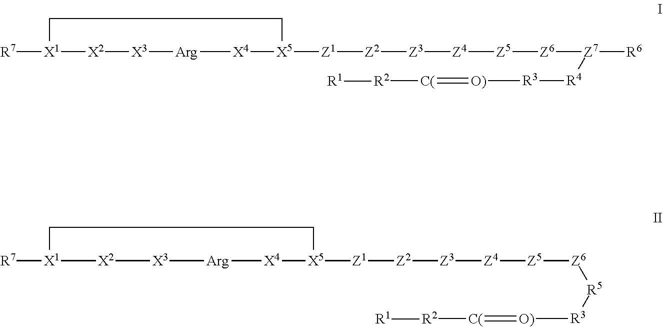

wherein
R¹ represents tetrazol-5-yl or carboxy;
R² represents a straight-chain, branched and/or cyclic C₆₋₂₀alkylene, C₆₋₂₀alkenylene or C₆₋₂₀alkynylene which may optionally be substituted with one or more substituents selected from halogen, hydroxy and aryl;
R³ is absent or represents —NH—S(═O)₂—(CH₂)₃₋₅—C(═O)— or a peptide fragment comprising one or two amino acid residues derived from natural or unnatural amino acids and containing at least one carboxy group;
wherein the side chains of R³ do not contain amino, guanidino, imidazolyl or other basic groups positively charged at neutral pH;
R⁴ in formula I represents a glycolether-based aminoacid structure according to one of the formulas IIIa-IIIh;

—HN—CH₂—CH₂—O—CH₂—CH₂—O—CH₂—C(═O)— [IIIa]

—[HN—CH₂—CH₂—O—CH₂—CH₂—O—CH₂—C(═O)]₂— [IIIb]

—[HN—CH₂—CH₂—O—CH₂—CH₂—O—CH₂—C(═O)]₃₋₅— [IIIc]

—[HN—CH₂—CH₂—O—CH₂—CH₂—O—CH₂—CH₂—NH—C(═O)—CH₂—CH₂—CH₂—C(═O)]₁₋₃— [IIId]

—[HN—CH₂—CH₂—O—CH₂—CH₂—O—CH₂—CH₂—NH—C(═O)—CH₂—O—CH₂—C(═O)]₁₋₃— [IIIe]

—[HN—CH₂—CH₂—O—CH₂—CH₂—O—CH₂—CH₂—O—CH₂—CH₂—O—CH₂—CH₂—C(═O)]₁₋₃— [IIIf]

—HN—CH₂—CH₂—[O—CH₂—CH₂]₂₋₁₂—O—CH₂—C(═O)— [IIIg]

—HN—CH₂—CH₂—[O—CH₂—CH₂]₄₋₁₂—O—CH₂—CH₂—C(═O)— [IIIh]

wherein the C-terminus of R⁴ is linked to the side chain of Z⁷;
R⁵ in formula II represents a glycolether-based diamine structure according to one of the formulas IVa-IVe;

—HN—CH₂—CH₂—O—CH₂—CH₂—O—CH₂—CH₂—NH—[IVa]

—HN—CH₂—CH₂—CH₂—O—CH₂—CH₂—O—CH₂—CH₂—CH₂—NH— [IVb]

—HN—CH₂—CH₂—CH₂—[O—CH₂—CH₂]₂—O—CH₂—CH₂—CH₂—NH— [IVc]

—HN—CH₂—CH₂—[O—CH₂—CH₂]₂₋₁₀—O—CH₂—CH₂—NH— [IVd]

—HN—CH₂—CH₂—CH₂—[O—CH₂—CH₂]₃₋₁₀—O—CH₂—CH₂—CH₂—NH— [IVe]

wherein R⁵ is linked to the C-terminus of Z⁶, if Z⁶ is present, or to the C-terminus of Z⁵, if Z⁶ is absent;
R⁶ in formula I represents OR′ or N(R′)₂, wherein each R′ independently represents hydrogen or represents C₁₋₆alkyl, C₂₋₆alkenyl or C₂₋₆alkynyl which may optionally be substituted with one or more hydroxy;
R⁷ represents C₁₋₆alkanoyl, C₂₋₆alkenoyl or C₂₋₆alkynoyl which may optionally be substituted with one or more hydroxy;
X¹ represents Glu, Asp, Cys, homoCys, Lys, Orn, Dab or Dap;
X² represents His, Ser, Thr, Gln, Asn, Cit, Hyp, Pro, Ala, or dipeptide fragment Glu-His or Asp-His;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine, (S)-2,3,4,9-tetrahydro-1H-β-carboline-3-carboxylic acid, or dipeptide fragment Trp-Gly;
X⁵ represents Glu, Asp, Cys, homoCys, Lys, Orn, Dab or Dap;
wherein X¹ and X⁵ are joined, rendering the compound cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;
Z¹ represents Pro, D-Pro, Hyp, D-Hyp, Ser, D-Ser, Thr, D-Thr, Gln, D-Gln, Asn, D-Asn, Cit, D-Cit, Ala or D-Ala;
Z² represents Pro, D-Pro, Hyp, D-Hyp, Ser, D-Ser, Thr, D-Thr, Gln, D-Gln, Asn, D-Asn, Cit, D-Cit, Ala or D-Ala;
wherein at least one of residues Z¹ and Z² is selected from Pro, D-Pro, Hyp or D-Hyp;
Z³ is absent or represents Arg, D-Arg, homoArg, D-homoArg, Lys, D-Lys, homoLys, D-homoLys, Orn, D-Orn, Dab, D-Dab, Dap, D-Dap, Dab(Me₂), D-Dab(Me₂), Dap(Me₂), D-Dap(Me₂), Dab(iPr), D-Dab(iPr), Dap(iPr), D-Dap(iPr), His, D-His, Ser, D-Ser, Thr, D-Thr, Asn, D-Asn, Gln, D-Gln, Cit, D-Cit, Ala, D-Ala, Gly or β-Ala;
Z⁴ represents Glu, D-Glu, Asp, D-Asp, Ser, D-Ser, Thr, D-Thr, Asn, D-Asn, Gln, D-Gln, Cit, D-Cit, Ala, D-Ala, Gly or β-Ala;
with the proviso that Z⁴ is selected from Glu, D-Glu, Asp and D-Asp, if Z³ is Arg, D-Arg, homoArg, D-homoArg, Lys, D-Lys, homoLys, D-homoLys, Orn, D-Orn, Dab, D-Dab, Dap, D-Dap, Dab(Me₂), D-Dab(Me₂), Dap(Me₂), D-Dap(Me₂), Dab(iPr), D-Dab(iPr), Dap(iPr) or D-Dap(iPr);
Z⁵ represents Lys(BCMA), D-Lys(BCMA), homoLys(BCMA), D-homoLys(BCMA), Orn(BCMA), D-Orn(BCMA), Dab(BCMA), D-Dab(BCMA), Dap(BCMA), D-Dap(BCMA), Lys(biscarboxymethyl), D-Lys(biscarboxymethyl), homoLys(biscarboxymethyl), D-homoLys(biscarboxymethyl), Orn(biscarboxymethyl), D-Orn(biscarboxymethyl), Dab(biscarboxymethyl), D-Dab(biscarboxymethyl), Dap(biscarboxymethyl), D-Dap(biscarboxymethyl), Glu, D-Glu, Asp or D-Asp;
Z⁶ is absent or represents a peptide fragment comprising one to four amino acid residues selected from Glu, D-Glu, Asp, D-Asp, Ser, D-Ser, Thr, D-Thr, Asn, D-Asn, Gln, D-Gln, Cit, D-Cit, Ala, D-Ala, Gly or β-Ala;
Z⁷ in formula I represents Lys, D-Lys, homoLys, D-homoLys, Orn, D-Orn, Dab, D-Dab, Dap, or D-Dap;
wherein the amino group in the side chain of Z⁷ is linked to R⁴ via an amide bond;
and pharmaceutically acceptable salts, prodrugs and solvates thereof.

The invention further relates to the use of compounds of the invention in therapy, to pharmaceutical compositions comprising compounds of the invention, and to the use of compounds of the invention in the manufacture of medicaments.

### DESCRIPTION OF THE INVENTION

Among further embodiments of compounds of the present invention are the following:

2. A compound according to embodiment 1, wherein
R² represents straight-chain α,ω-C₁₂₋₂₀alkylene, α,ω-C₁₂₋₂₀alkenylene or α,ω-C₁₂₋₂₀alkynylene which may optionally be substituted with one or more hydroxyl;
R³ is absent or represents —NH—S(═O)₂—(CH₂)₃—C(═O)—, Glu, D-Glu, γ-Glu or D-γ-Glu;
R⁴ in formula I represents a glycolether-based aminoacid structure according to one of the formulas IIIa, IIIb and IIIc;
R⁵ in formula II represents a glycolether-based diamine structure according to one of the formulas IVa, IVb and IVc;
R⁶ in formula I represents OR′ or N(R′)₂, wherein each R′ independently represents hydrogen or C₁₋₃alkyl;
R⁷ represents C₁₋₃alkanoyl;
X³ represents D-Phe;
and X⁴ represents Trp or Trp-Gly.
3. A compound according to any of embodiments 1-2, wherein the compound is in accordance with formula I.
4. A compound according to any of embodiments 1-2, wherein the compound is in accordance with formula II.
5. A compound according to any of embodiments 1-4, wherein R¹-R² represents 13-(tetrazol-5-yl)tridecyl, 14-(tetrazol-5-yl)tetradecyl, 15-(tetrazol-5-yl)pentadecyl, 16-(tetrazol-5-yl)hexadecyl, 17-(tetrazol-5-yl)heptadecyl or 18-(tetrazol-5-yl)octadecyl.
6. A compound according to any of embodiments 1-4, wherein R¹-R² represents 15-(tetrazol-5-yl)pentadecyl.
7. A compound according to any of embodiments 1-4, wherein R¹-R² represents 16-(tetrazol-5-yl)hexadecyl.
8. A compound according to any of embodiments 1-4, wherein R¹-R² represents 13-carboxytridecyl, 14-carboxytetradecyl, 15-carboxypentadecyl, 16-carboxyhexadecyl, 17-carboxyheptadecyl, 18-carboxyoctadecyl or 19-carboxynonadecyl.
9. A compound according to any of embodiments 1-4, wherein R¹-R² represents 14-carboxytetradecyl, 16-carboxyhexadecyl or 18-carboxyoctadecyl.
10. A compound according to any of embodiments 1-4, wherein R¹-R² represents 14-carboxytetradecyl.
11. A compound according to any of embodiments 1-4, wherein R¹-R² represents 15-carboxypentadecyl.
12. A compound according to any of embodiments 1-4, wherein R¹-R² represents 16-carboxyhexadecyl.
13. A compound according to any of embodiments 1-4, wherein R¹-R² represents 18-carboxyoctadecyl.
14. A compound according to any of embodiments 1-13, wherein R³ is absent.
15. A compound according to any of embodiments 1-13, wherein R³ represents —NH—S(═O)₂—(CH₂)₃—C(═O)—.
16. A compound according to any of embodiments 1-13, wherein R³ represents γ-Glu.
17. A compound according to any of embodiments 1-3 and 5-16, wherein R⁴ represents a structure according to formula IIIa.
18. A compound according to any of embodiments 1-3 and 5-16, wherein R⁴ represents a structure according to formula IIIb.
19. A compound according to any of embodiments 1-3 and 5-16, wherein R⁴ represents a structure according to formula IIIc.
20. A compound according to any of embodiments 1-2 and 4-16, wherein R⁵ represents a structure according to formula IVa.
21. A compound according to any of embodiments 1-2 and 4-16, wherein R⁵ represents a structure according to formula IVb.
22. A compound according to any of embodiments 1-2 and 4-16, wherein R⁵ represents a structure according to formula IVc.
23. A compound according to any of embodiments 1-3 and 5-19, wherein R⁶ is NH₂.
24. A compound according to any of embodiments 1-3 and 5-19, wherein R⁶ is OH.
25. A compound according to any of embodiments 1-24, wherein R⁷ is acetyl.
26. A compound according to any of embodiments 1-25, wherein X¹ is Lys, Orn, Dab or Dap and X⁵ is Glu or Asp.
27. A compound according to any of embodiments 1-25, wherein X¹ is Glu or Asp and X⁵ is Lys, Orn, Dab or Dap.
28. A compound according to any of embodiments 1-25, wherein X¹ is homoCys and X⁵ is Cys.
29. A compound according to any of embodiments 1-25, wherein X¹ is homoCys and X⁵ is homoCys.
30. A compound according to any of embodiments 1-25, wherein X¹ is Cys and X⁵ is Cys.
31. A compound according to any of embodiments 1-25, wherein X¹ is Cys and X⁵ is homoCys.
32. A compound according to any of embodiments 1-25, wherein X¹ is Lys and X⁵ is Asp.
33. A compound according to any of embodiments 1-25, wherein X¹ is Orn and X⁵ is Asp.
34. A compound according to any of embodiments 1-25, wherein X¹ is Dab and X⁵ is Asp.
35. A compound according to any of embodiments 1-25, wherein X¹ is Dap and X⁵ is Asp.
36. A compound according to any of embodiments 1-25, wherein X¹ is Orn and X⁵ is Glu.
37. A compound according to any of embodiments 1-25, wherein X¹ is Dab and X⁵ is Glu.
38. A compound according to any of embodiments 1-25, wherein X¹ is Dap and X⁵ is Glu.
39. A compound according to any of embodiments 1-25, wherein X¹ is Asp and X⁵ is Lys.
40. A compound according to any of embodiments 1-25, wherein X¹ is Asp and X⁵ is Orn.
41. A compound according to any of embodiments 1-25, wherein X¹ is Asp and X⁵ is Dab.
42. A compound according to any of embodiments 1-25, wherein X¹ is Asp and X⁵ is Dap.
43. A compound according to any of embodiments 1-25, wherein X¹ is Glu and X⁵ is Lys.
44. A compound according to any of embodiments 1-25, wherein X¹ is Glu and X⁵ is Orn.
45. A compound according to any of embodiments 1-25, wherein X¹ is Glu and X⁵ is Dab.
46. A compound according to any of embodiments 1-24, wherein X¹ is Glu and X⁵ is Dap.
47. A compound according to any of embodiments 1-46, wherein X² is His.
48. A compound according to any of embodiments 1-46, wherein X² is Glu-His.
49. A compound according to any of embodiments 1-46, wherein X² is Asp-His.
50. A compound according to any of embodiments 1-46, wherein X² is Hyp.
51. A compound according to any of embodiments 1-46, wherein X² is Pro.
52. A compound according to any of embodiments 1-46, wherein X² is Ser, Thr, Gln, Asn or Cit.
53. A compound according to any of embodiments 1-46, wherein X² is Ser. 54. A compound according to any of embodiments 1-46, wherein X² is Gln.
55. A compound according to any of embodiments 1-54, wherein X⁴ is Trp.
56. A compound according to any of embodiments 1-55, wherein Z¹ is Pro, Hyp, D-Pro or D-Hyp.
57. A compound according to any of embodiments 1-55, wherein Z¹ is Ser.
58. A compound according to any of embodiments 1-55, wherein Z¹ is Pro.
59. A compound according to any of embodiments 1-55, wherein Z¹ is Hyp.
60. A compound according to any of embodiments 1-55, wherein Z¹ is D-Pro.
61. A compound according to any of embodiments 1-55, wherein Z¹ is D-Hyp.
62. A compound according to any of embodiments 1-61, wherein Z² is Pro, Hyp, D-Pro or D-Hyp.
63. A compound according to any of embodiments 1-61, wherein Z² is Ser.
64. A compound according to any of embodiments 1-60, wherein Z² is Pro.
65. A compound according to any of embodiments 1-60, wherein Z² is Hyp.
66. A compound according to any of embodiments 1-60, wherein Z² is D-Pro.
67. A compound according to any of embodiments 1-60, wherein Z² is D-Hyp.
68. A compound according to any of embodiments 1-67, wherein Z³ is Lys, homoLys, Orn, Dab or Dap.
69. A compound according to any of embodiments 1-67, wherein Z³ is Lys, homoLys or Orn.
70. A compound according to any of embodiments 1-67, wherein Z³ is Lys.
71. A compound according to any of embodiments 1-67, wherein Z³ is Arg or homoArg.
72. A compound according to any of embodiments 1-67, wherein Z³ is Arg.
73. A compound according to any of embodiments 1-67, wherein Z³ is His.
74. A compound according to any of embodiments 1-67, wherein Z³ is Ser.
75. A compound according to any of embodiments 1-67, wherein Z³ is absent.
76. A compound according to any of embodiments 1-75, wherein Z⁴ is Glu or Asp.
77. A compound according to any of embodiments 1-75, wherein Z⁴ is Glu.
78. A compound according to any of embodiments 1-75, wherein Z⁴ is Asp.
79. A compound according to any of embodiments 1-67 and 73-75, wherein Z⁴ is Ser, Thr, Asn, Gln or Cit.
80. A compound according to any of embodiments 1-67 and 73-75, wherein Z⁴ is Ser.
81. A compound according to any of embodiments 1-80, wherein Z⁵ is Lys(BCMA), homoLys(BCMA), Orn(BCMA), Dab(BCMA), Dap(BCMA), Lys(biscarboxymethyl), homoLys(biscarboxymethyl), Orn(biscarboxymethyl), Dab(biscarboxymethyl) or Dap(biscarboxymethyl).
82. A compound according to any of embodiments 1-80, wherein Z⁵ is Lys(biscarboxymethyl), homoLys(biscarboxymethyl) or Orn(biscarboxymethyl).
83. A compound according to any of embodiments 1-80, wherein Z⁵ is Lys(biscarboxymethyl).
84. A compound according to any of embodiments 1-80, wherein Z⁵ is Lys(BCMA), homoLys(BCMA), Orn(BCMA), Dab(BCMA) or Dap(BCMA).
85. A compound according to any of embodiments 1-80, wherein Z⁵ is Lys(BCMA).
86. A compound according to any of embodiments 1-80, wherein Z⁵ is Dap(BCMA).
87. A compound according to any of embodiments 1-80, wherein Z⁵ is Glu or Asp.
88. A compound according to any of embodiments 1-80, wherein Z⁵ is Glu.
89. A compound according to any of embodiments 1-88, wherein Z⁶ is absent or represents a peptide fragment comprising one to three amino acid residues selected from Glu, D-Glu, Asp, D-Asp, Ser, D-Ser, Thr, D-Thr, Asn, D-Asn, Gln, D-Gln, Cit, D-Cit, Ala, D-Ala, Gly or β-Ala.
90. A compound according to any of embodiments 1-88, wherein Z⁶ is absent.
91. A compound according to any of embodiments 1-88, wherein Z⁶ represents a peptide fragment comprising one to three amino acid residues selected from Glu and Ser.
92. A compound according to any of embodiments 1-88, wherein Z⁶ is Glu.
93. A compound according to any of embodiments 1-88, wherein Z⁶ is Ser-Ser-Glu.
94. A compound according to any of embodiments 1-88, wherein Z⁶ is Ser-Ser.
95. A compound according to any of embodiments 1-88, wherein Z⁶ is Glu-Ser-Ser.
96. A compound according to any of embodiments 1-88, wherein Z⁶ is Ser-Glu-Ser.
97. A compound according to any of embodiments 1-88, wherein Z⁶ is Ser.
98. A compound according to any of embodiments 1-3, 5-19 and 23-97, wherein Z⁷ is Lys, homoLys or Orn.
99. A compound according to any of embodiments 1-3, 5-19 and 23-97, wherein Z⁷ is Lys.
100. A compound according to embodiment 1, selected from the group consisting of:
Ac-c[Dap-His-D-Phe-Arg-Trp-Asp]-Pro-Pro-Arg-Asp-Lys(BCMA)-Lys[(2-{2-[16-(tetrazol-5-yl)hexadecanoylamino]ethoxy}ethoxy)acetyl]-NH₂

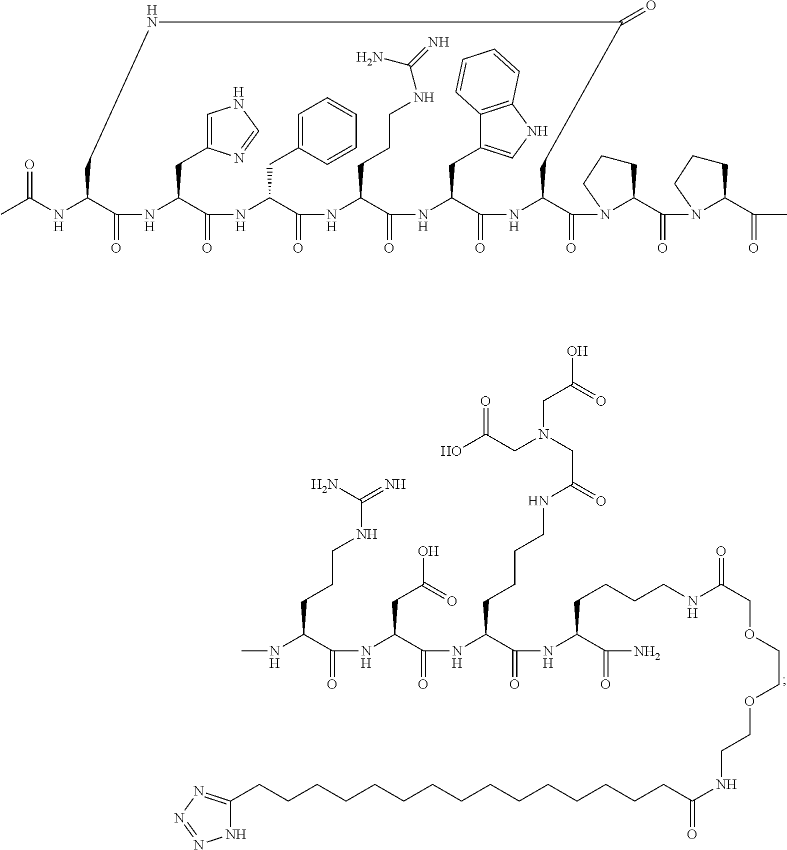 [SMILES: CC(=O)N[C@H]1CNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Asp-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

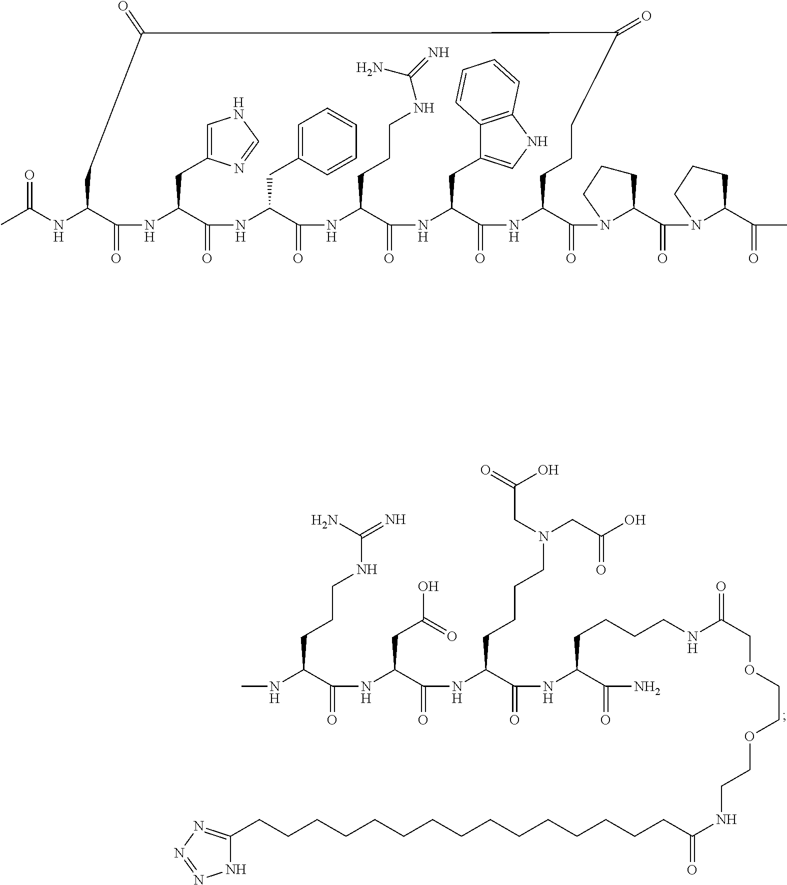 [SMILES: CC(=O)N[C@H]1CC(=O)C(=O)CCC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Dab-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl)

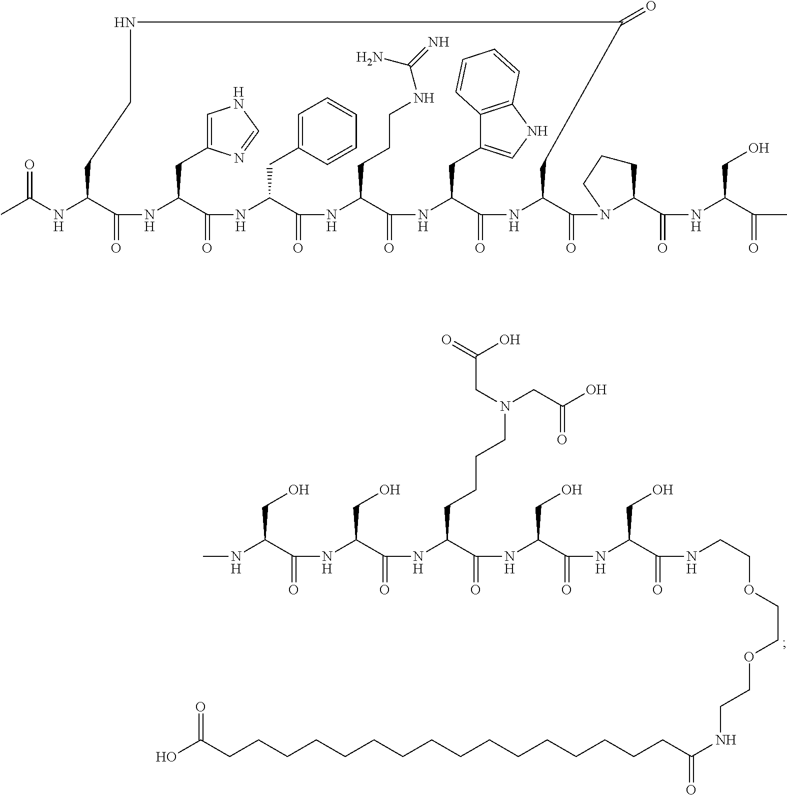 [SMILES: CC(=O)N[C@H]1CCNC(=O)C[C@@H](C(=O)N2CCC[C@H]2C(=O)N[C@@H](CO)C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]

Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-OH

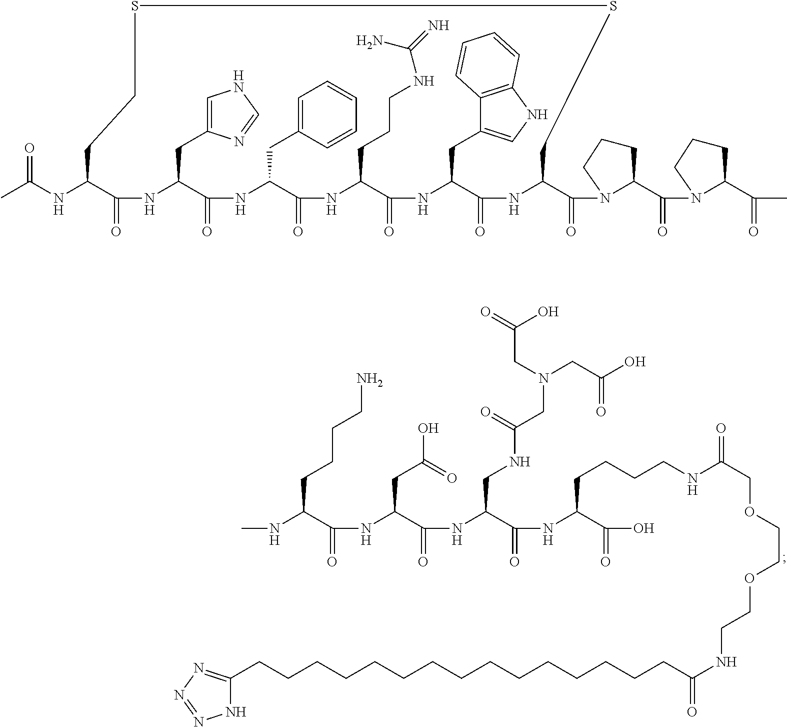 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(=O)O]

Ac-c[homoCys-His-D-Phe-Arg-Trp-Cys]-Pro-Pro-Lys-Glu-Lys(biscarboxymethyl)-Lys({2-[2-(15-carboxypentadecanoylamino)ethoxy]ethoxy}acetyl)-NH₂

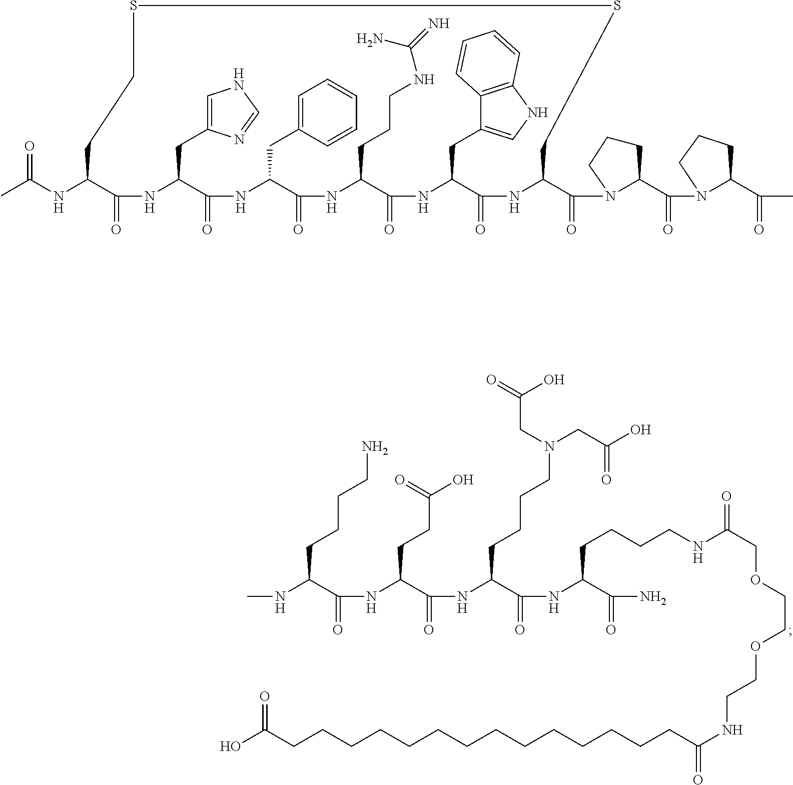 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

Ac-c[Glu-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

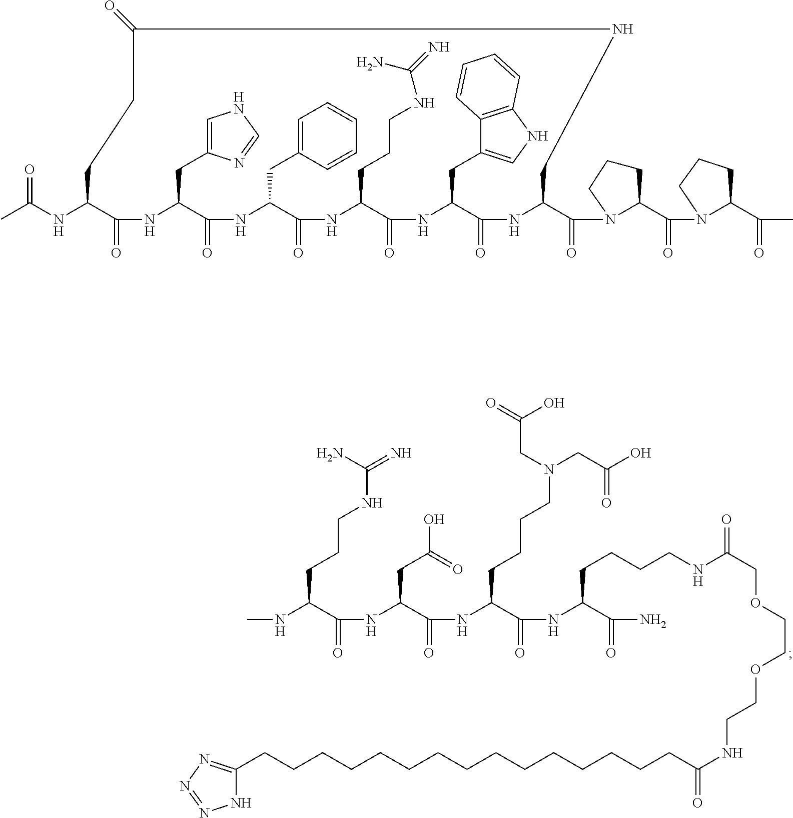 [SMILES: CC(=O)N[C@H]1CCC(=O)NC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Lys-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂

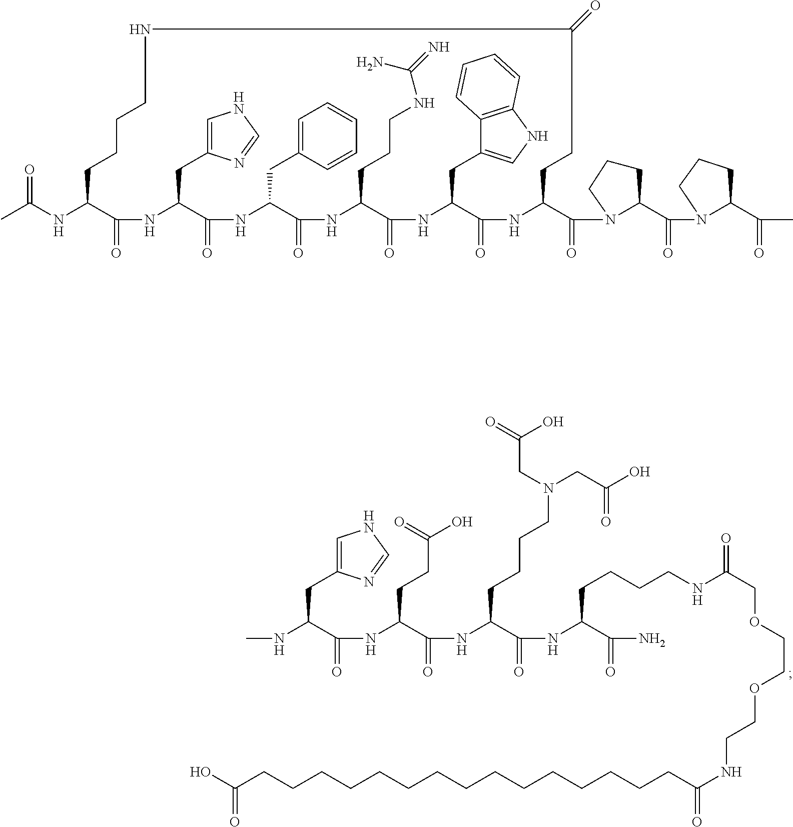 [SMILES: CC(=O)N[C@H]1CCCCNC(=O)CC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]

Ac-c[Orn-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

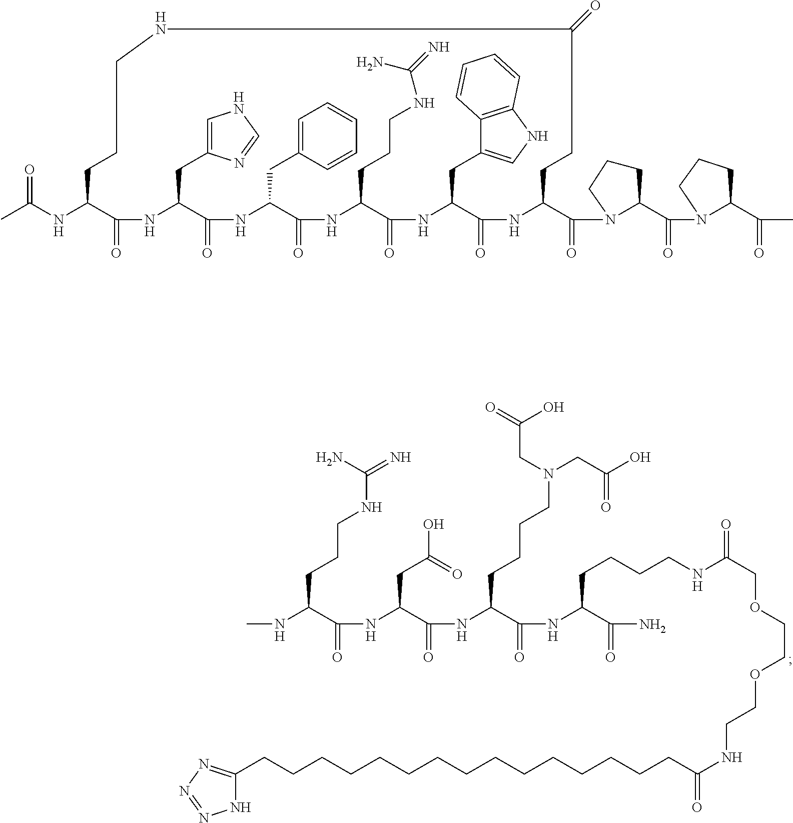 [SMILES: CC(=O)N[C@H]1CCCNC(=O)CC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Dab-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl)

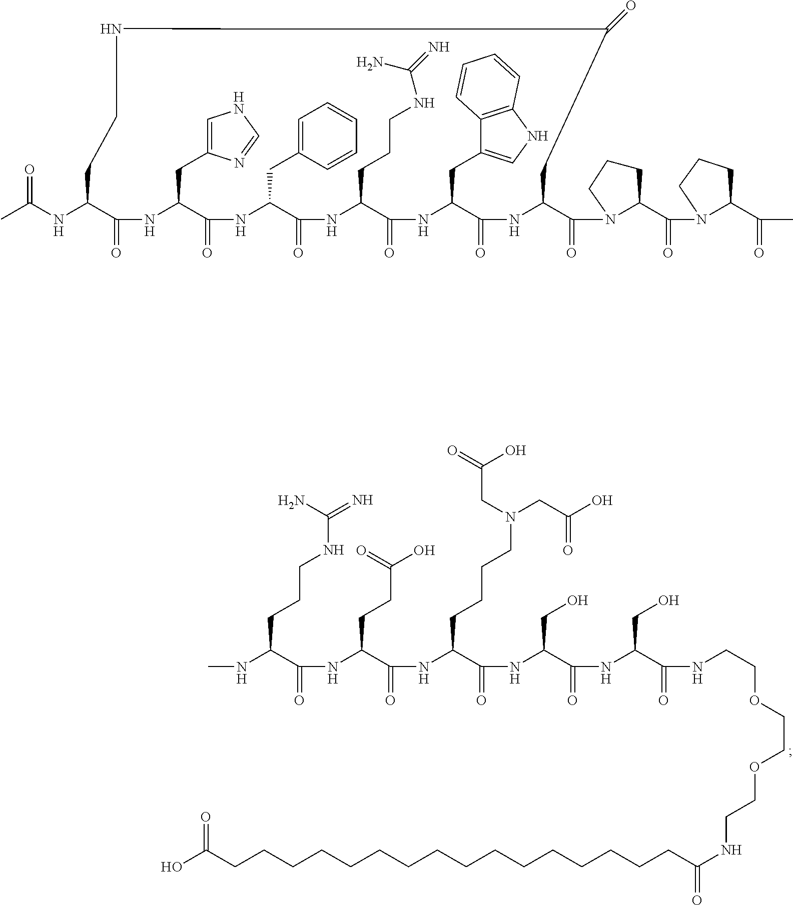 [SMILES: CC(=O)N[C@H]1CCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CCC(=O)O)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]

Ac-c[Orn-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂

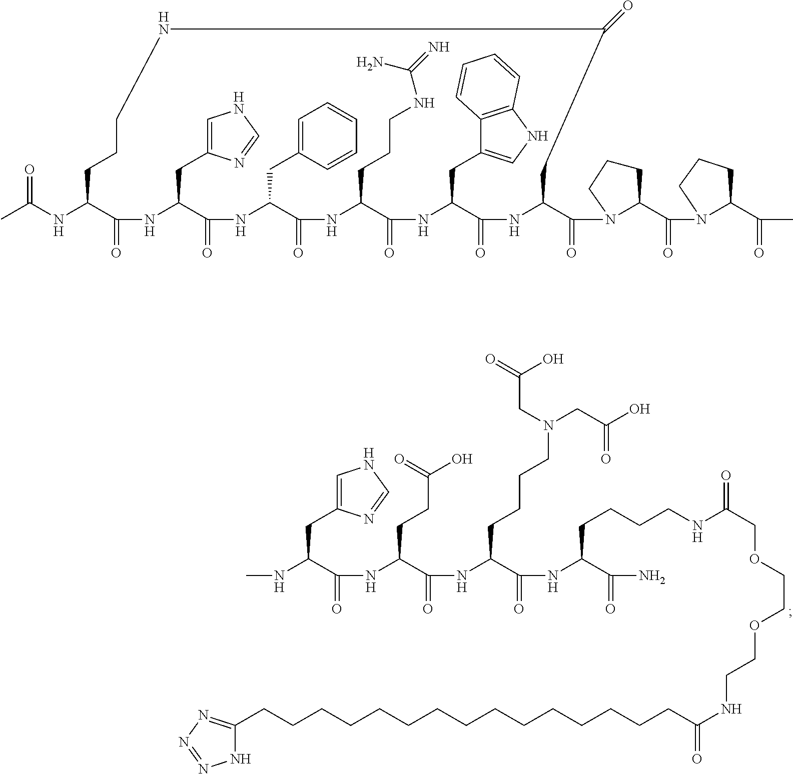 [SMILES: CC(=O)N[C@H]1CCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Lys-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

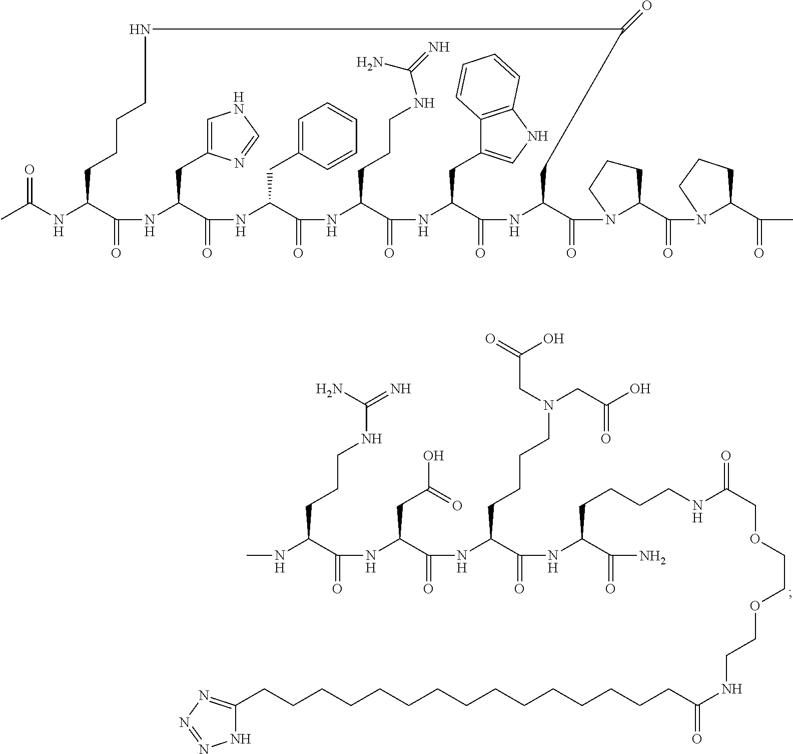 [SMILES: CC(=O)N[C@H]1CCCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Orn-His-D-Phe-Arg-Trp-Asp]-Pro-Ser-Ser-Ser-Lys(biscarboxymethyl)-Ser-Ser-Lys({2-[2-(15-carboxypentadecanoylamino)ethoxy]ethoxy}acetyl)-NH₂

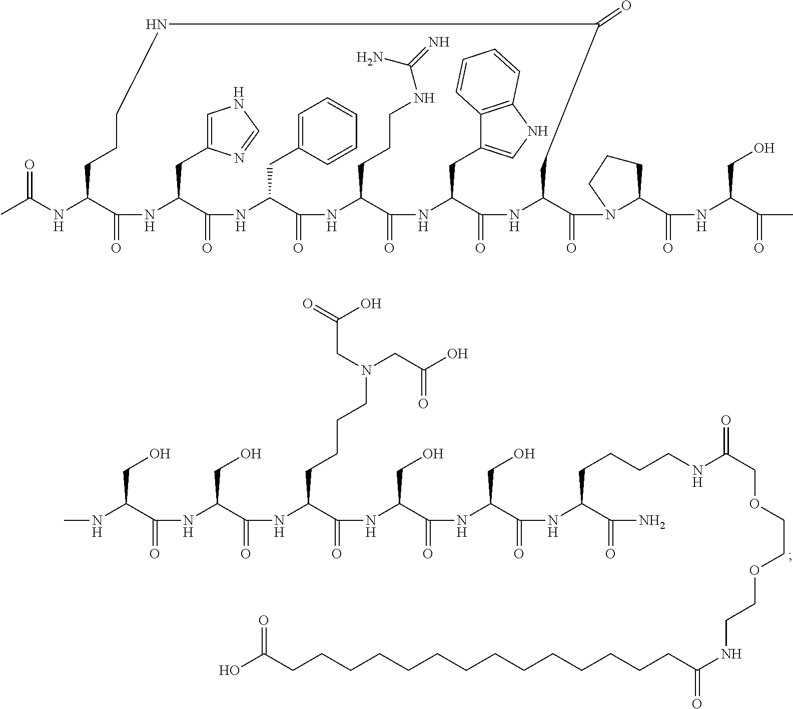

Ac-c[Glu-His-D-Phe-Arg-Trp-Orn]-Pro-Pro-Asp-Lys(biscarboxymethyl)-Lys({2-[2-(15-carboxypentadecanoylamino)ethoxy]ethoxy}acetyl)-NH₂

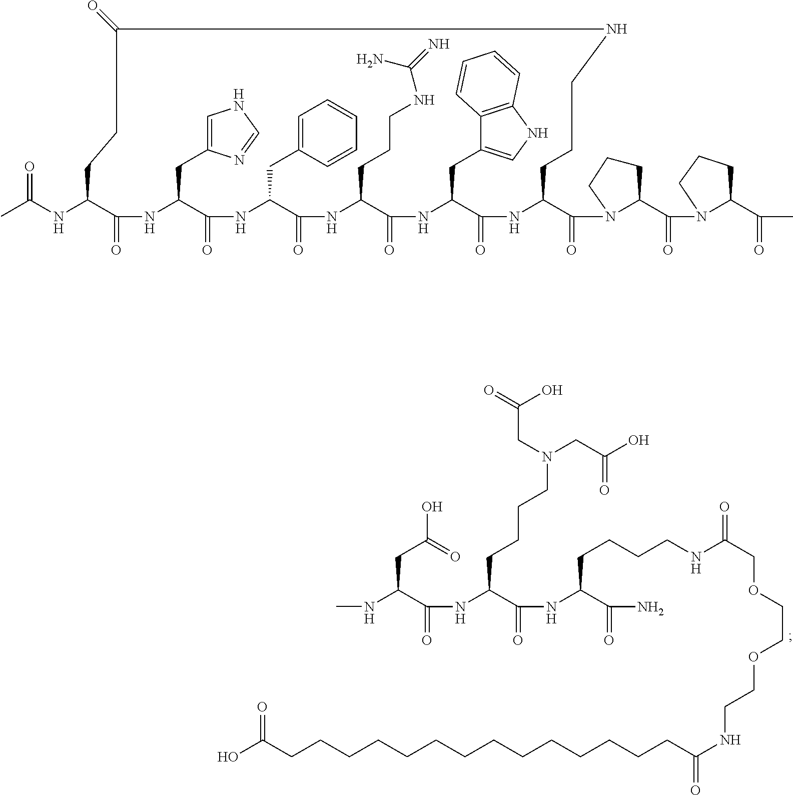 [SMILES: CC(=O)N[C@H]1CCC(=O)NCCC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

Ac-c[Orn-His-D-Phe-Arg-Trp-Asp]-Pro-Ser-Ser-Ser-Lys(biscarboxymethyl)-Ser-Ser-Lys({2-[2-(17-carboxyheptadecanoylamino)ethoxy]ethoxy}acetyl)-NH₂

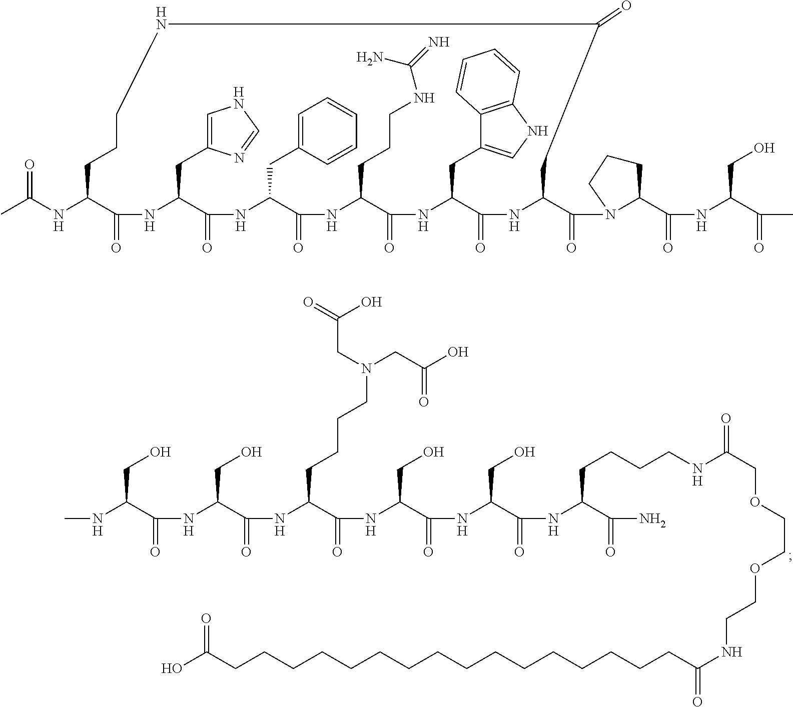

Ac-c[Dab-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂

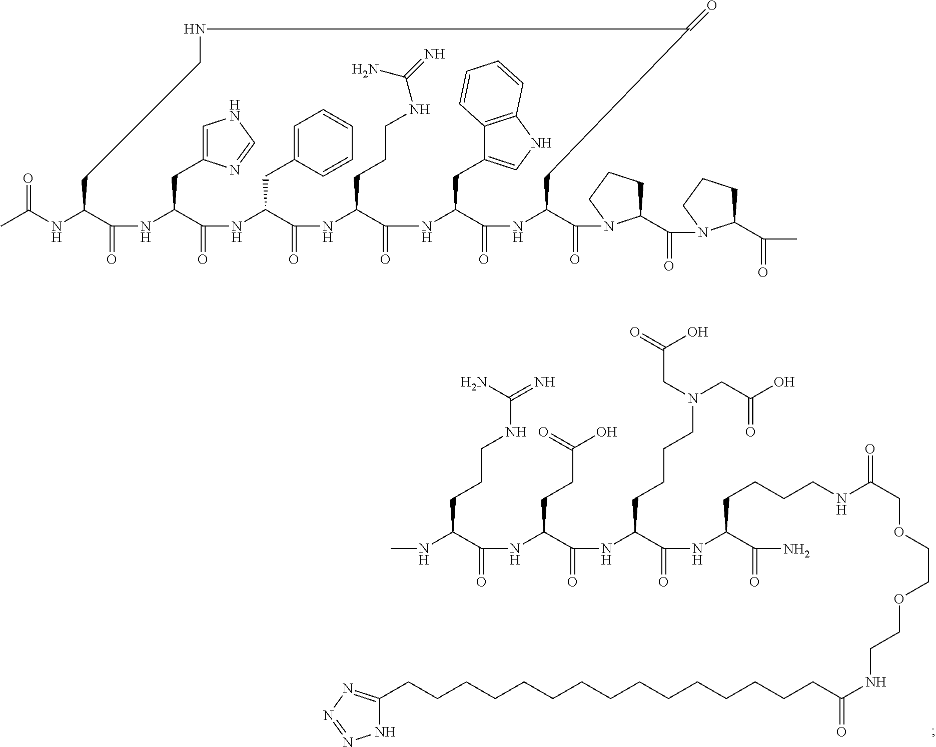 [SMILES: CC(=O)N[C@H]1CCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Glu-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

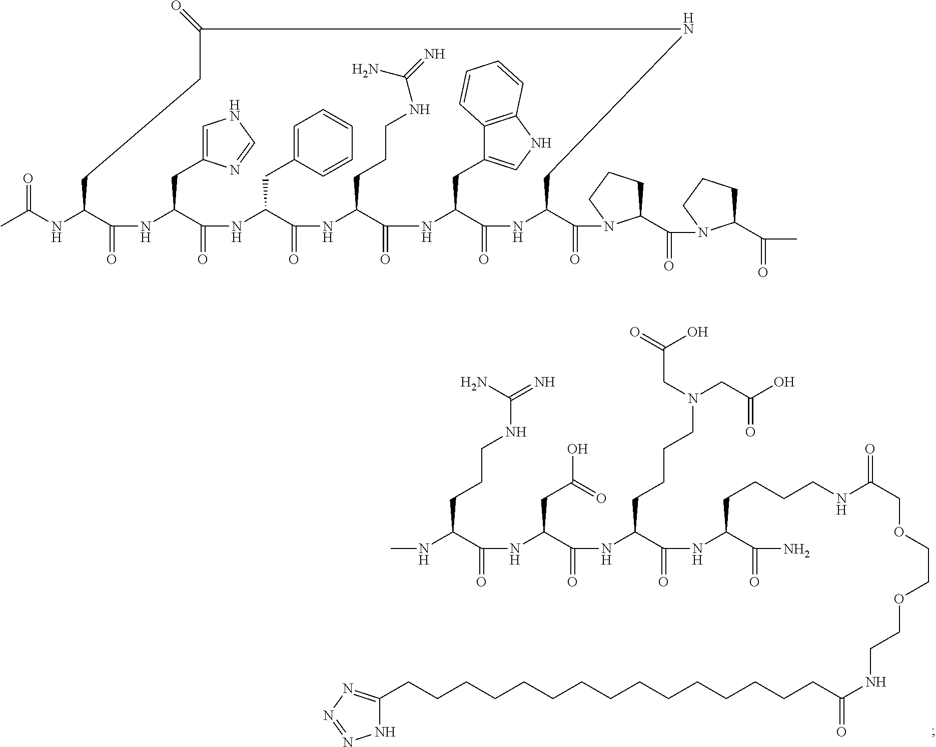 [SMILES: CC(=O)N[C@H]1CCC(=O)NC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Orn-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

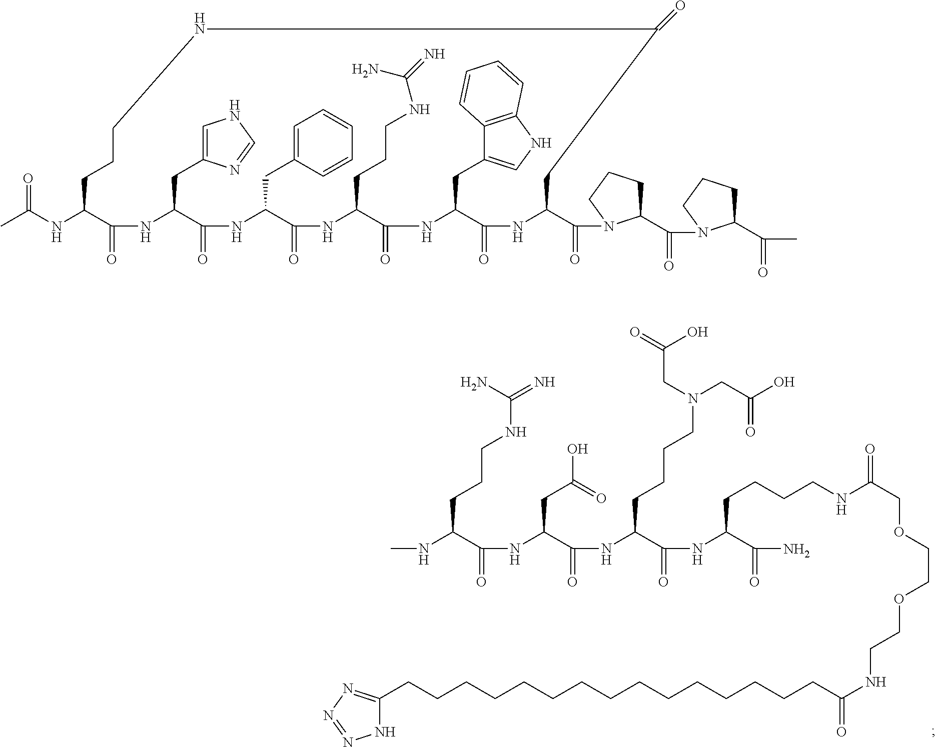 [SMILES: CC(=O)N[C@H]1CCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Glu-His-D-Phe-Arg-Trp-Orn]-Pro-Pro-Arg-Asp-Lys(biscarboxymethyl)-Lys({2-[2-(15-carboxypentadecanoylamino)ethoxy]ethoxy}acetyl)-NH₂

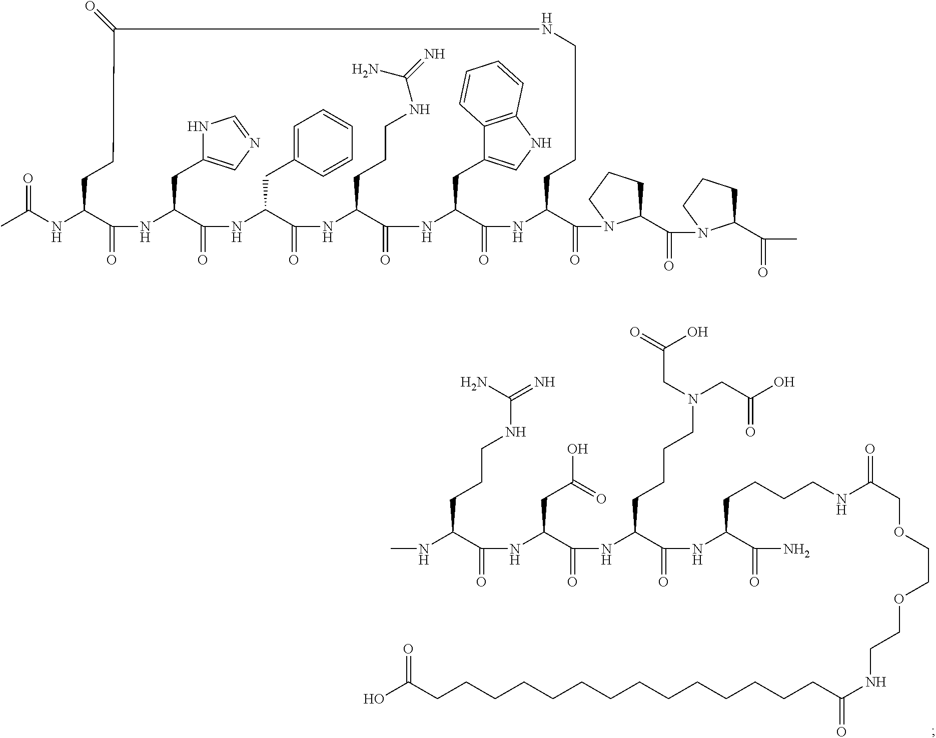 [SMILES: CC(=O)N[C@H]1CCC(=O)NCCC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O ; ";"]

Ac-c[Orn-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂

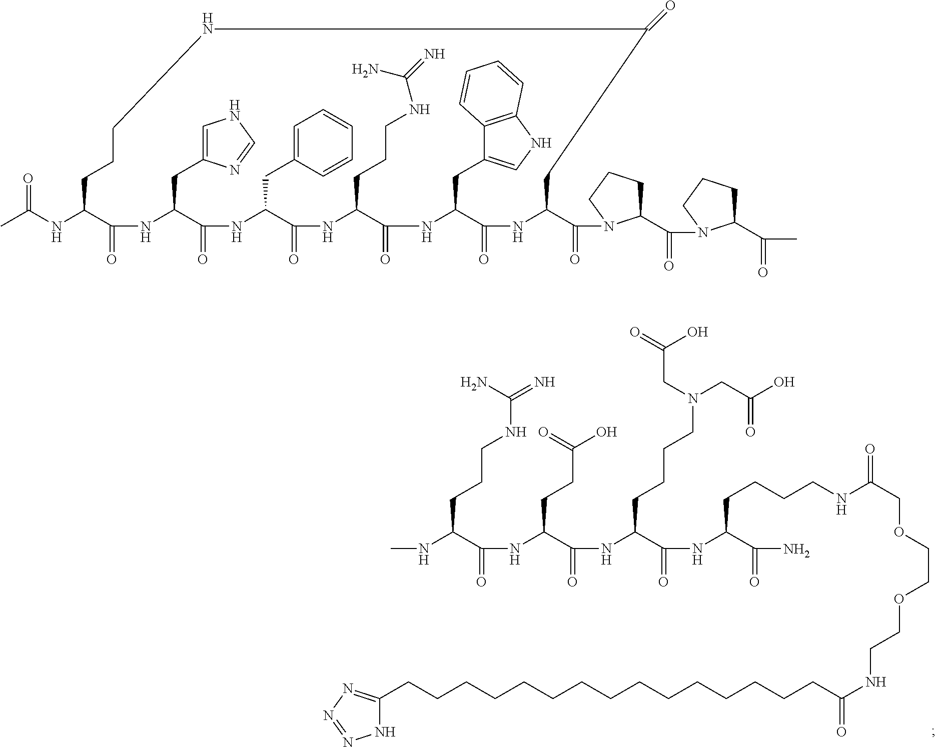 [SMILES: CC(=O)N[C@H]1CCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Orn-His-D-Phe-Arg-Trp-Glu]-Pro-Ser-Lys-Glu-Glu-Ser-Ser-Glu-Lys({2-[2-(16-carboxyhexadecanoylamino)ethoxy]ethoxy}acetyl)-NH₂

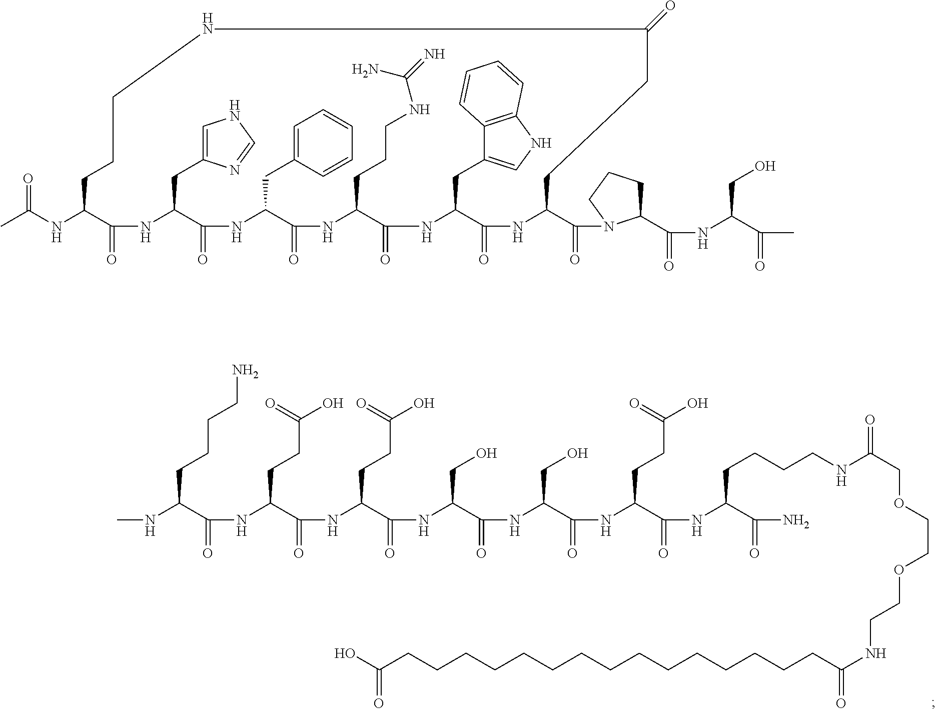

Ac-c[Lys-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

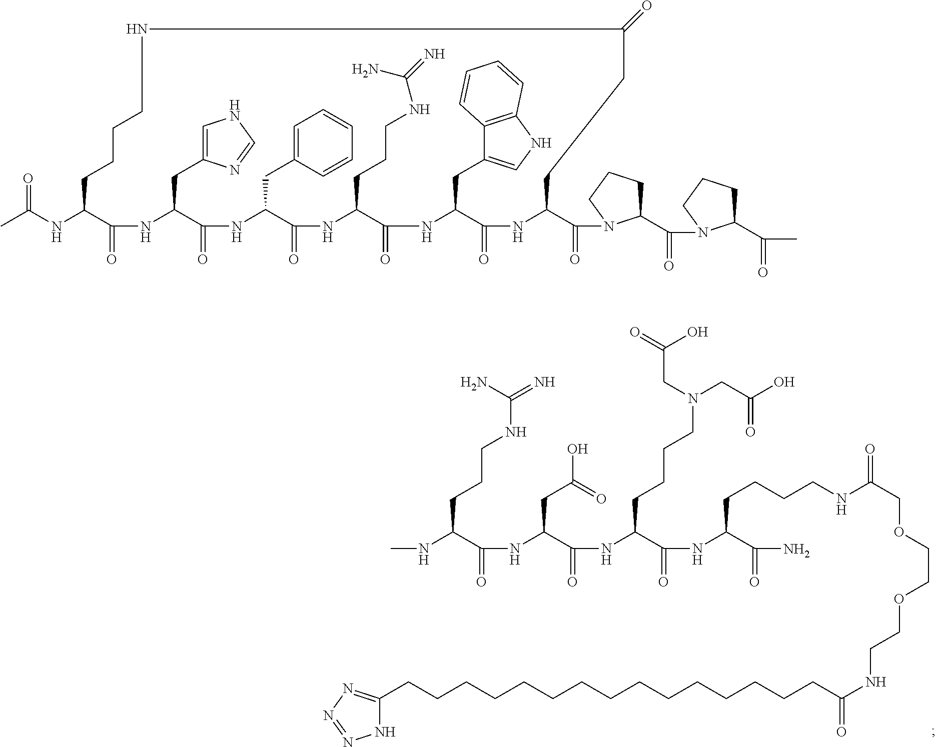 [SMILES: CC(=O)N[C@H]1CCCCNC(=O)CC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Orn-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂

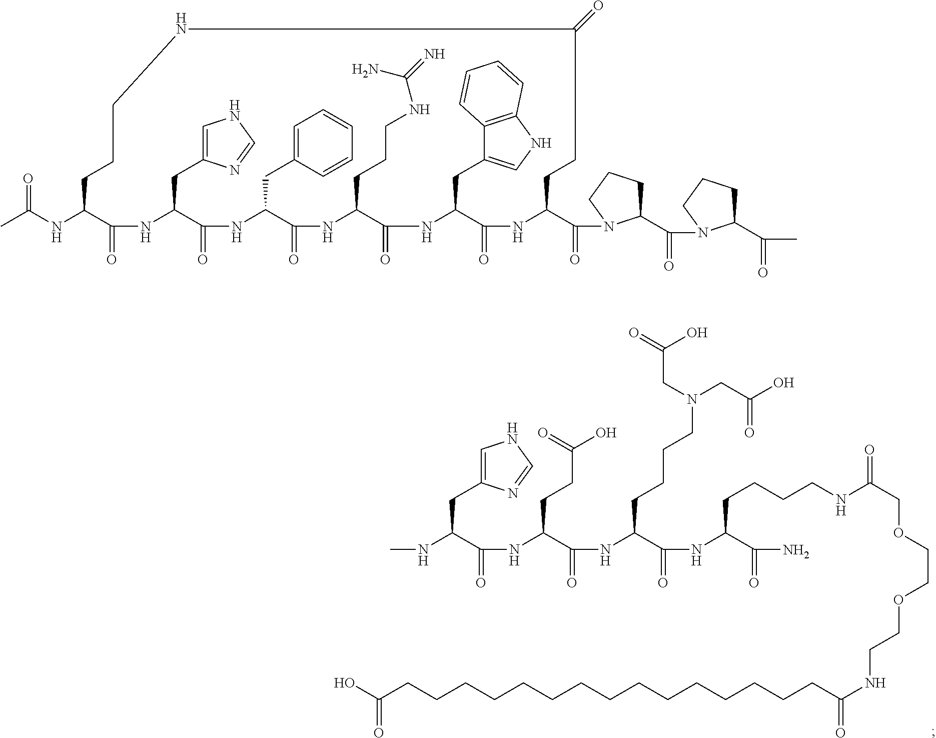 [SMILES: CC(=O)N[C@H]1CCCNC(=O)CC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O ; ";"]

Ac-c[Dab-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

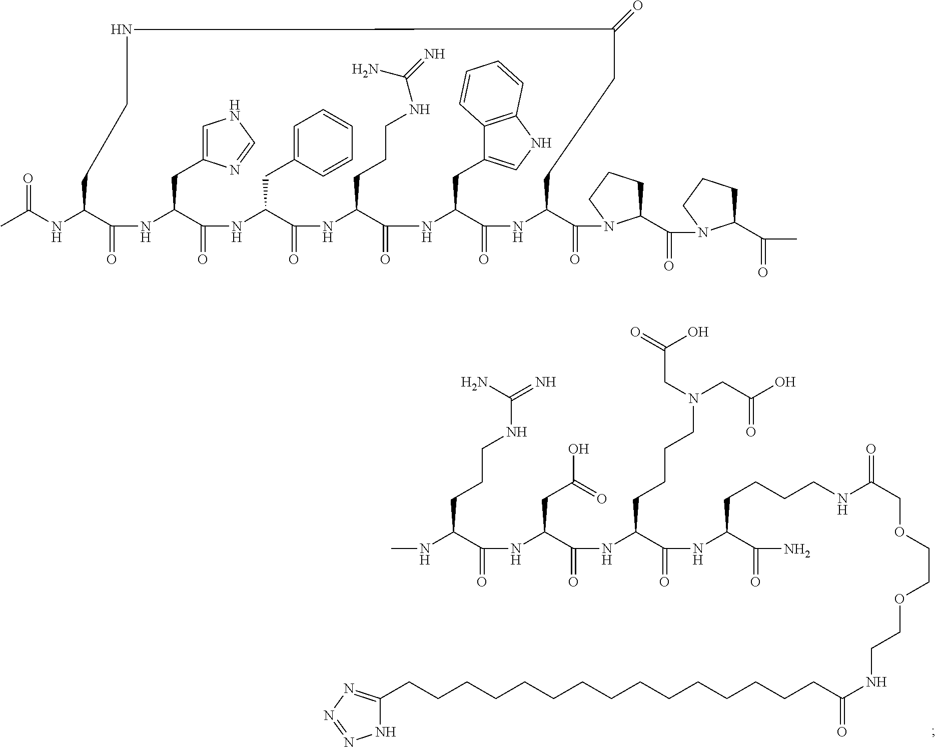 [SMILES: CC(=O)N[C@H]1CCNC(=O)CC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Asp-His-D-Phe-Arg-Trp-Dap]-Pro-Pro-Arg-Asp-Lys(BCMA)-Lys[(2-{2-[16-(tetrazol-5-yl)hexadecanoylamino]ethoxy}ethoxy)acetyl]-NH₂

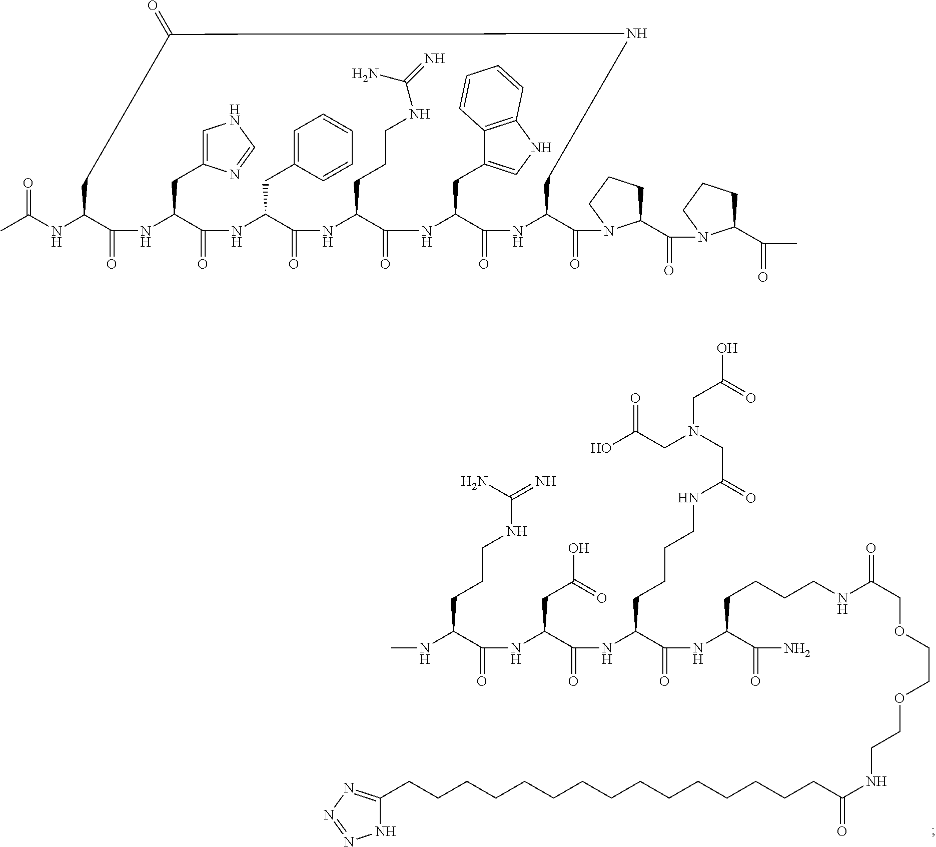 [SMILES: CC(=O)N[C@H]1CC(=O)NC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Dab-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

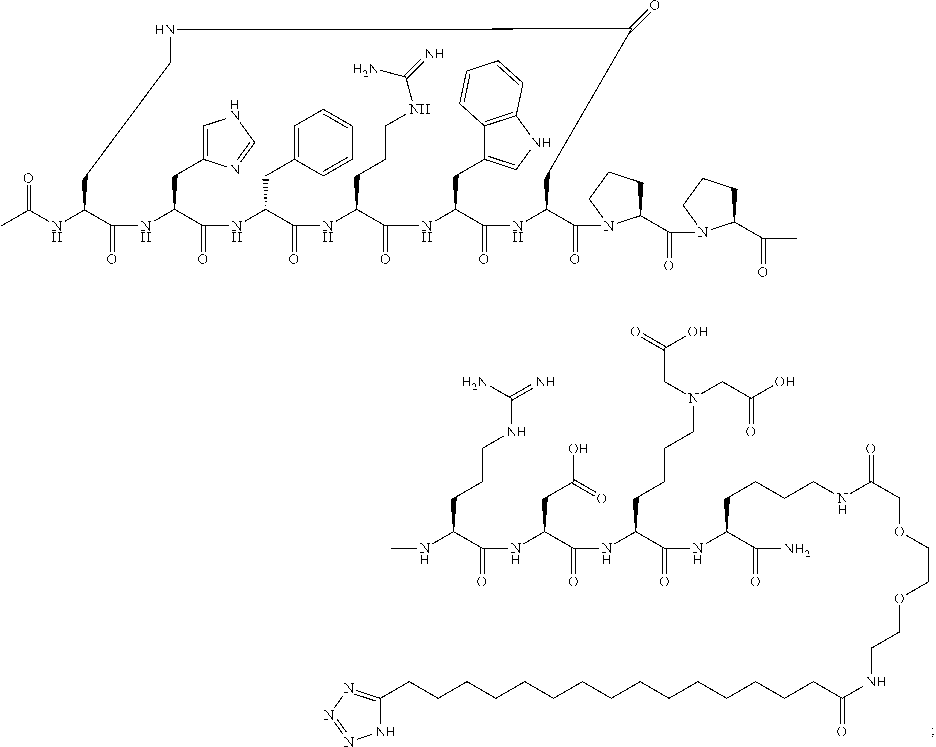 [SMILES: CC(=O)N[C@H]1CCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Glu-His-D-Phe-Arg-Trp-Orn]-Pro-Pro-Asp-Lys(biscarboxymethyl)-Lys({2-[2-(17-carboxyheptadecanoylamino)ethoxy]ethoxy}acetyl)-NH₂

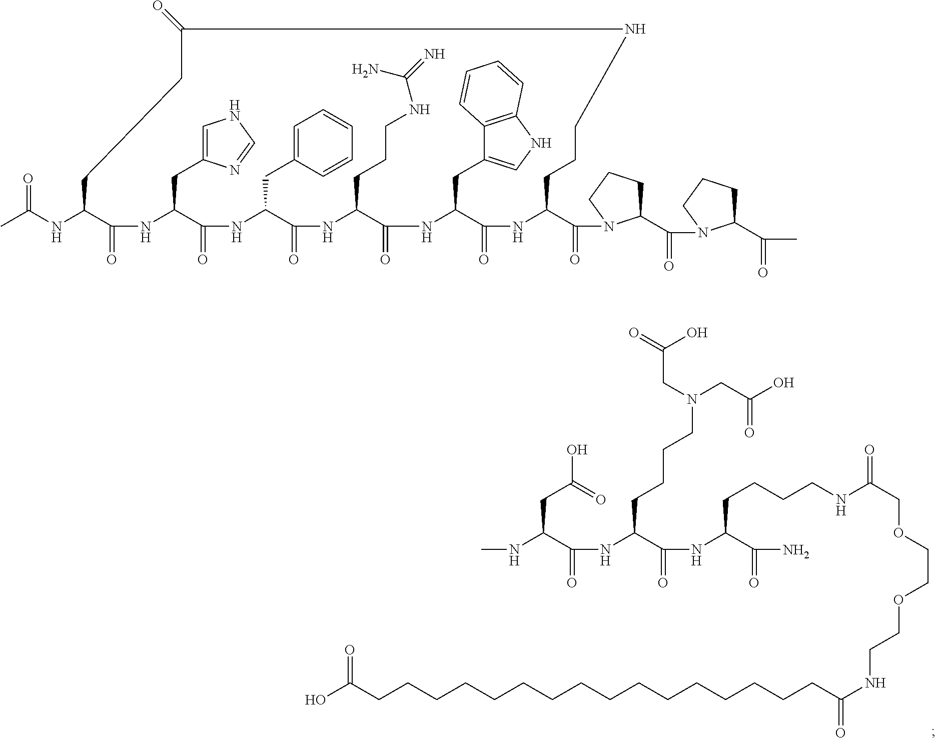 [SMILES: CC(=O)N[C@H]1CCC(=O)NCCC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O ; ";"]

Ac-c[Glu-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

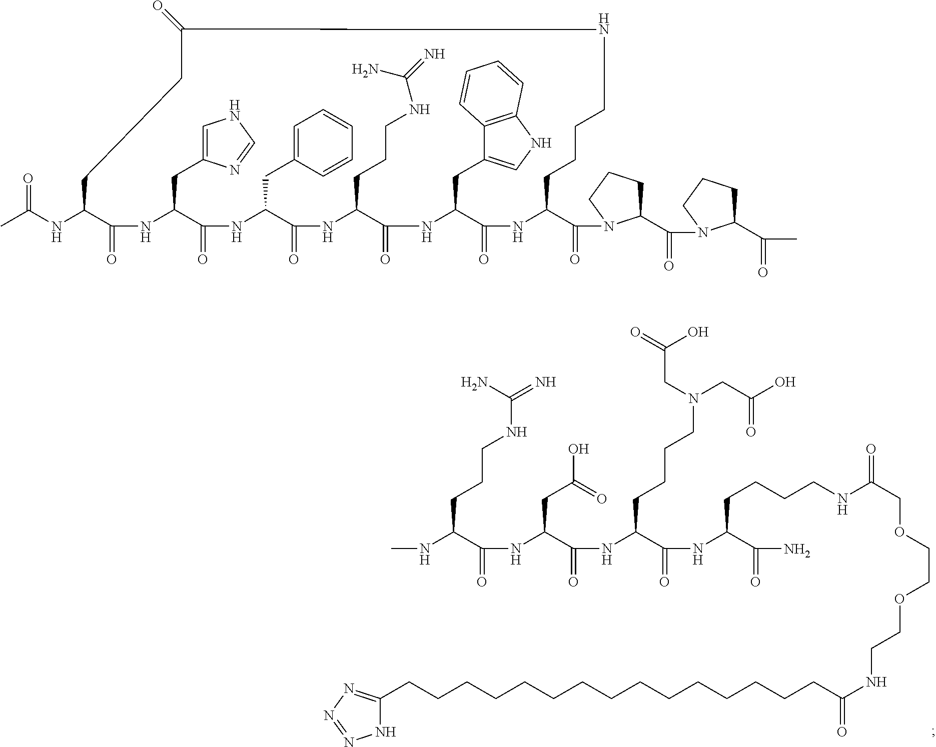 [SMILES: CC(=O)N[C@H]1CCC(=O)NCCCC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Orn-His-D-Phe-Arg-Trp-Asp]-Pro-Ser-Ser-Ser-Lys(biscarboxymethyl)-Ser-Ser-Lys[(2-{2-[16-(tetrazol-5-yl)hexadecanoylamino]ethoxy}ethoxy)acetyl]-NH₂

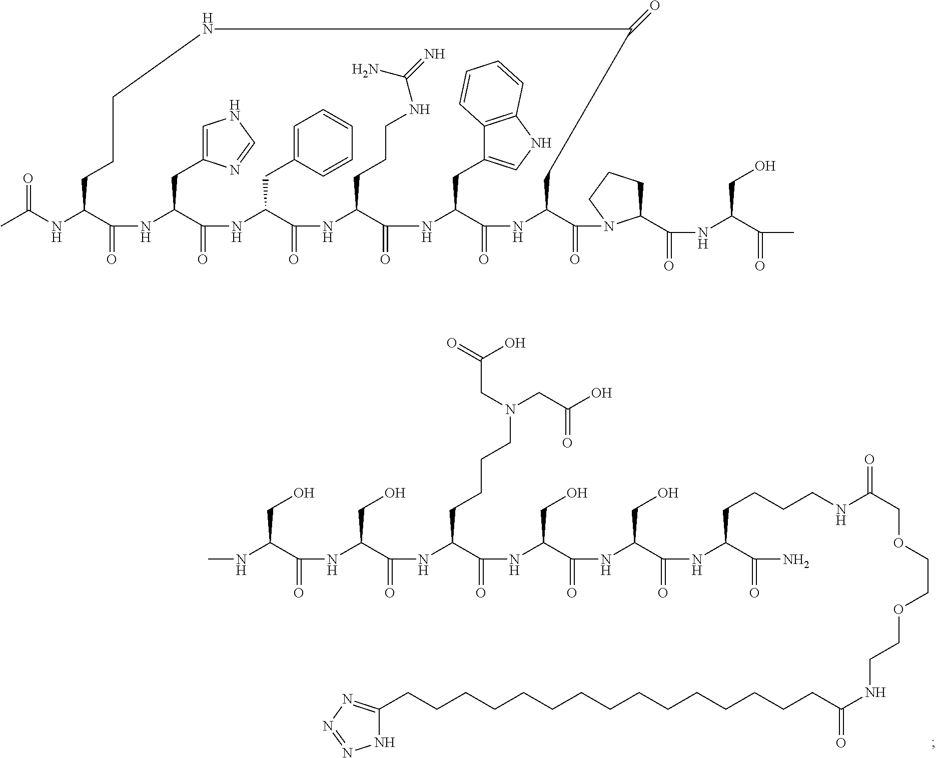

Ac-c[Glu-His-D-Phe-Arg-Trp-Orn]-D-Pro-D-Pro-Arg-Asp-Lys(biscarboxymethyl)-Lys[(2-{2-[16-(tetrazol-5-yl)hexadecanoylamino]ethoxy}ethoxy)acetyl]-NH₂

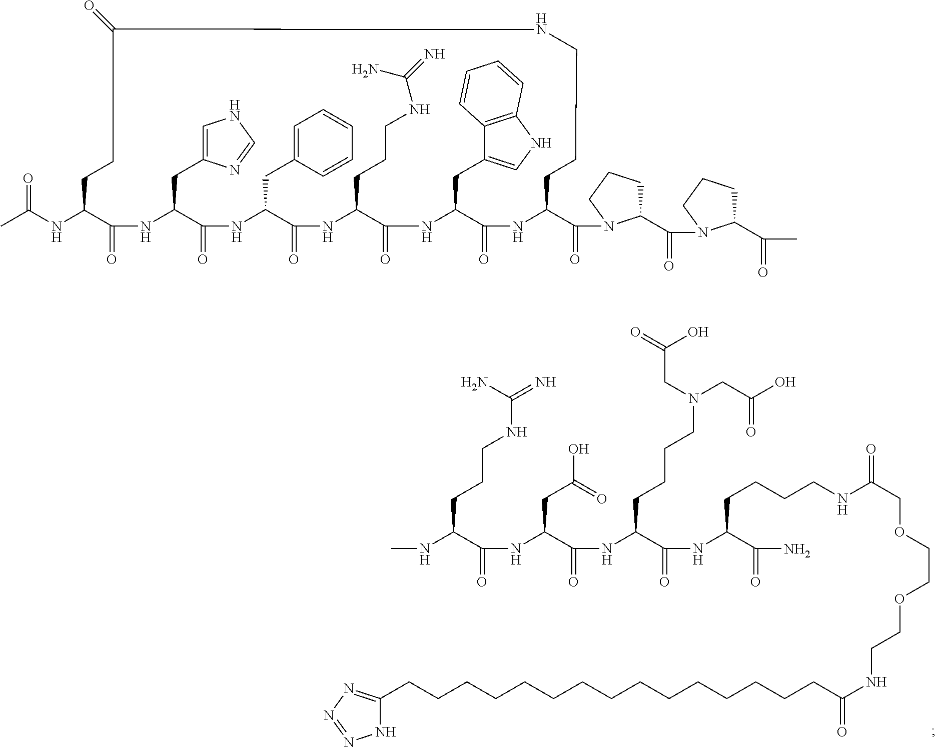 [SMILES: CC(=O)N[C@H]1CCC(=O)NCCC[C@@H](C(=O)N2CCC[C@@H]2C(=O)N2CCC[C@@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O ; ";"]

Ac-c[Asp-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

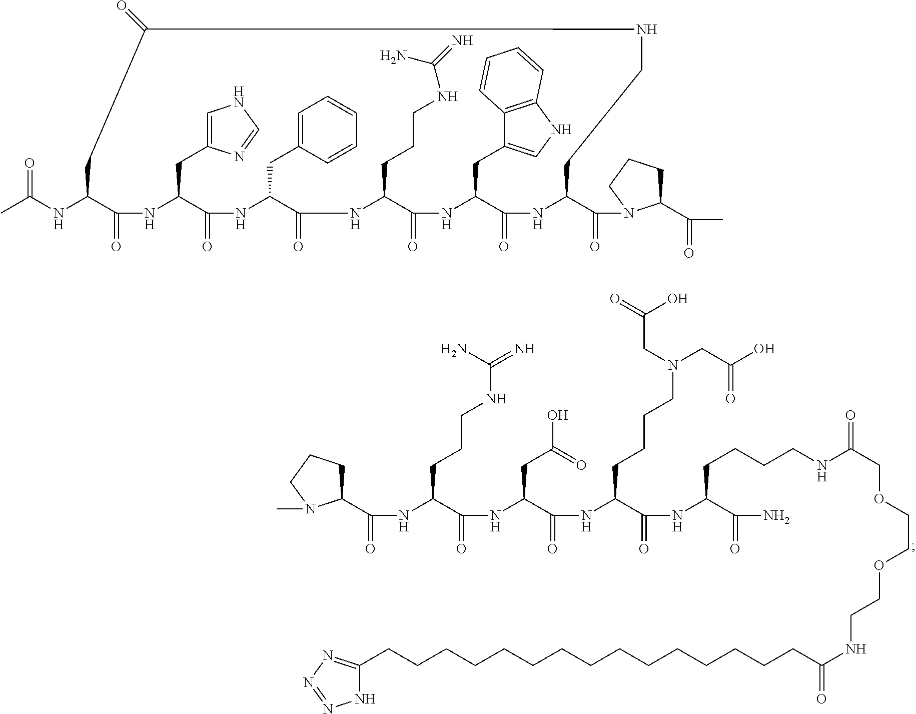

Ac-c[Glu-His-D-Phe-Arg-Trp-Dap]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

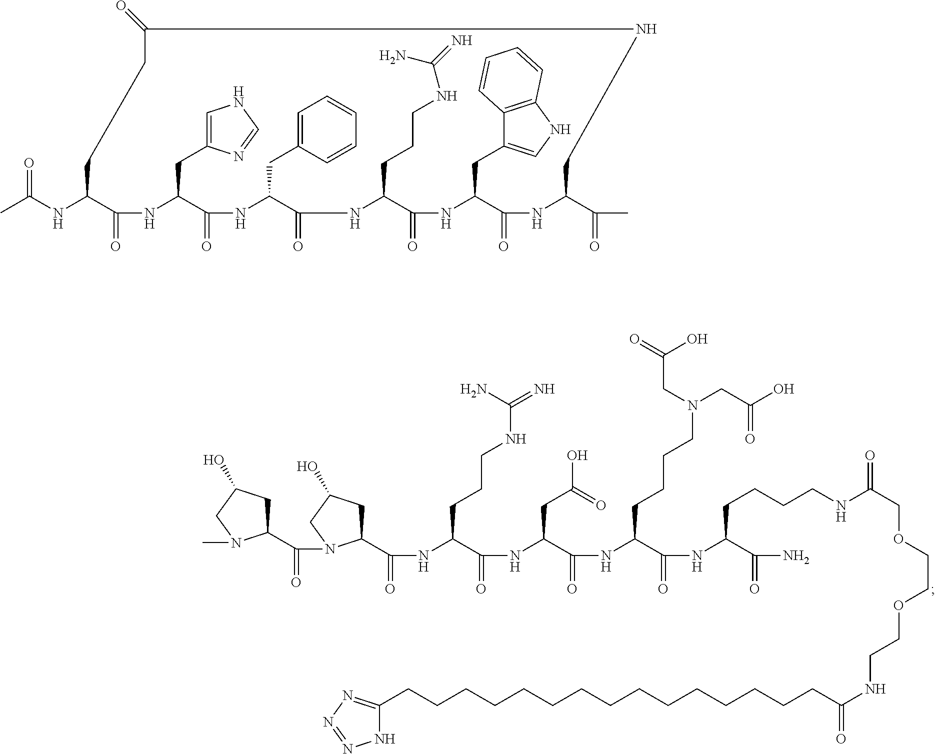 [SMILES: CC(=O)N[C@H]1CCC(=O)NC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1C[C@H](O)C[C@H]1C(=O)N1C[C@H](O)C[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Glu-His-D-Phe-Arg-Trp-Dab]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

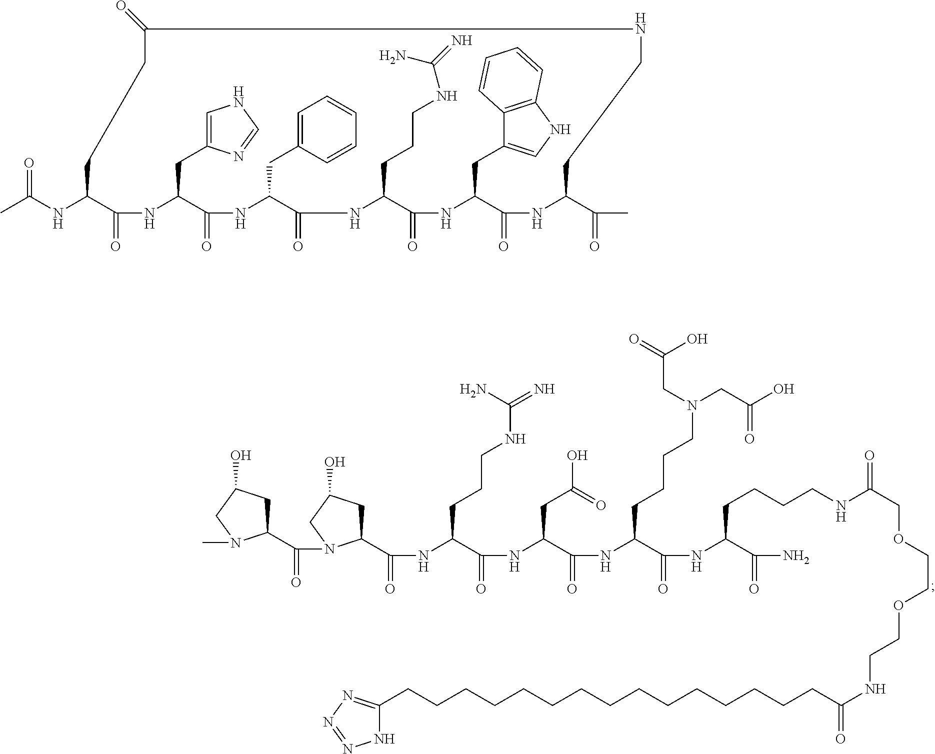 [SMILES: CC(=O)N[C@H]1CCC(=O)NCC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1C[C@H](O)C[C@H]1C(=O)N1C[C@H](O)C[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Dab-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂

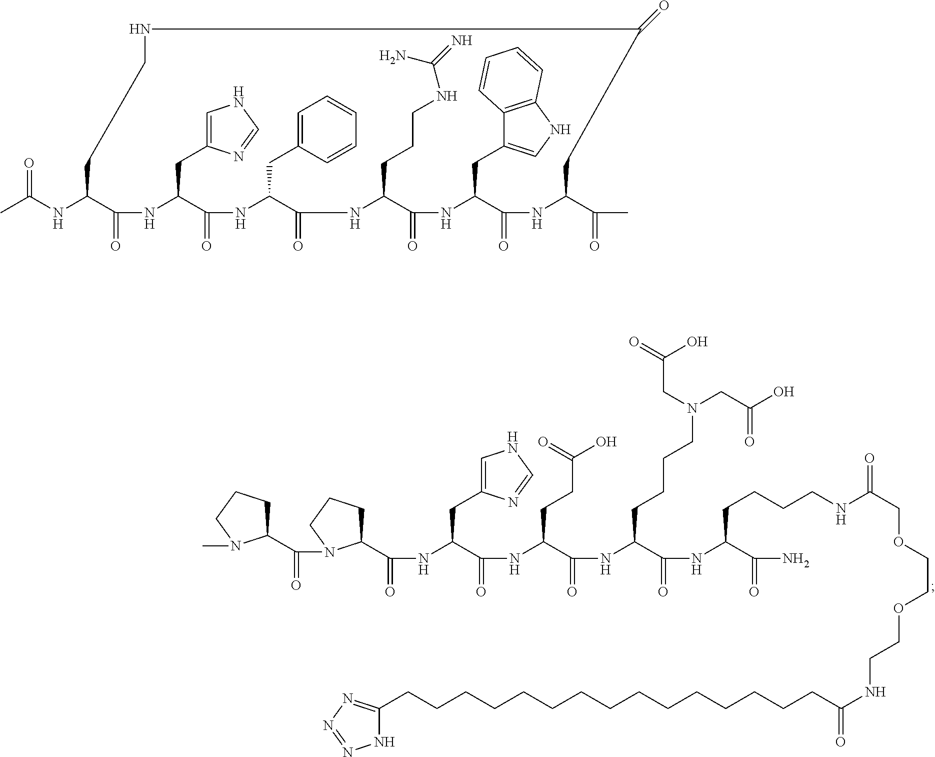 [SMILES: CC(=O)N[C@H]1CCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Orn-His-D-Phe-Arg-Trp-Asp]-Pro-Pro-Arg-Glu-Lys(biscarboxymethyl)-Lys({2-[2-(17-carboxyheptadecanoylamino)ethoxy]ethoxy}acetyl)-NH₂

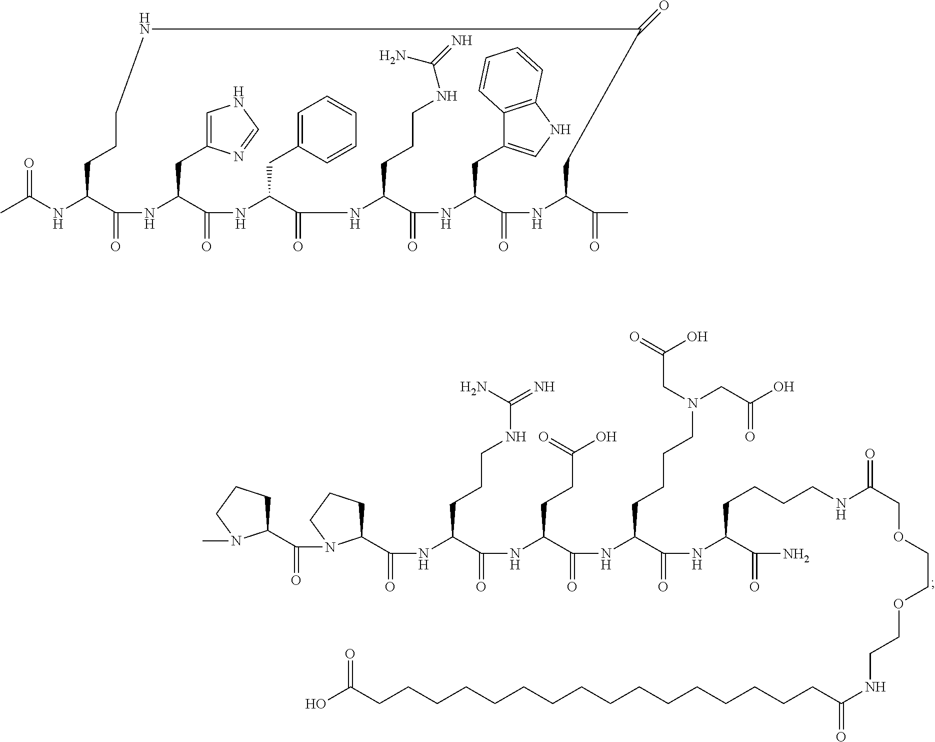 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]

Ac-c[Asp-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

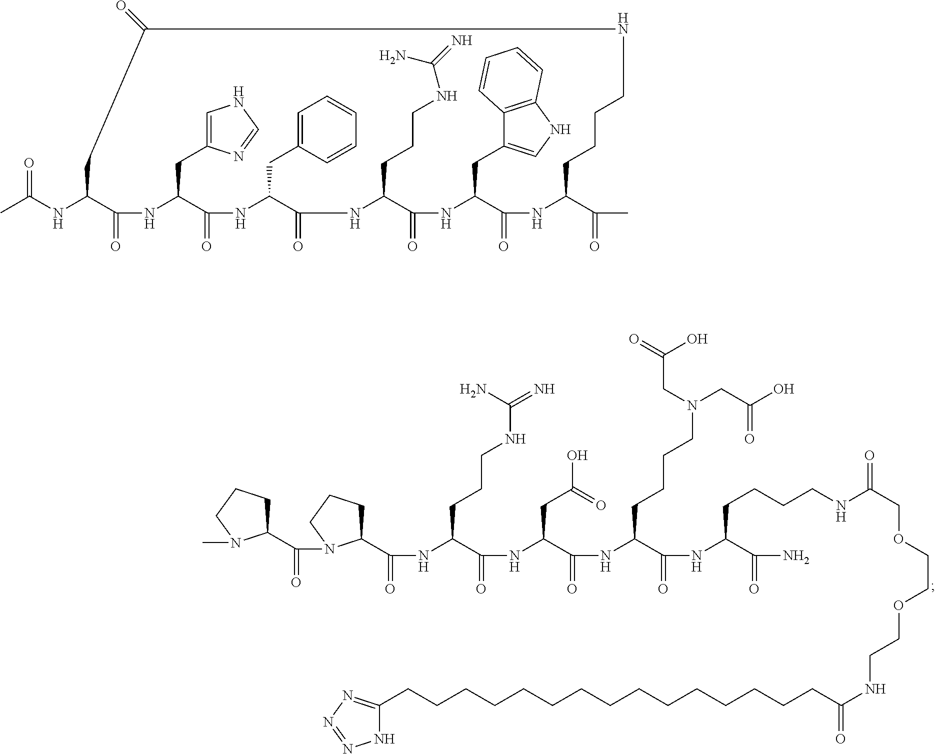

Ac-c[Dap-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

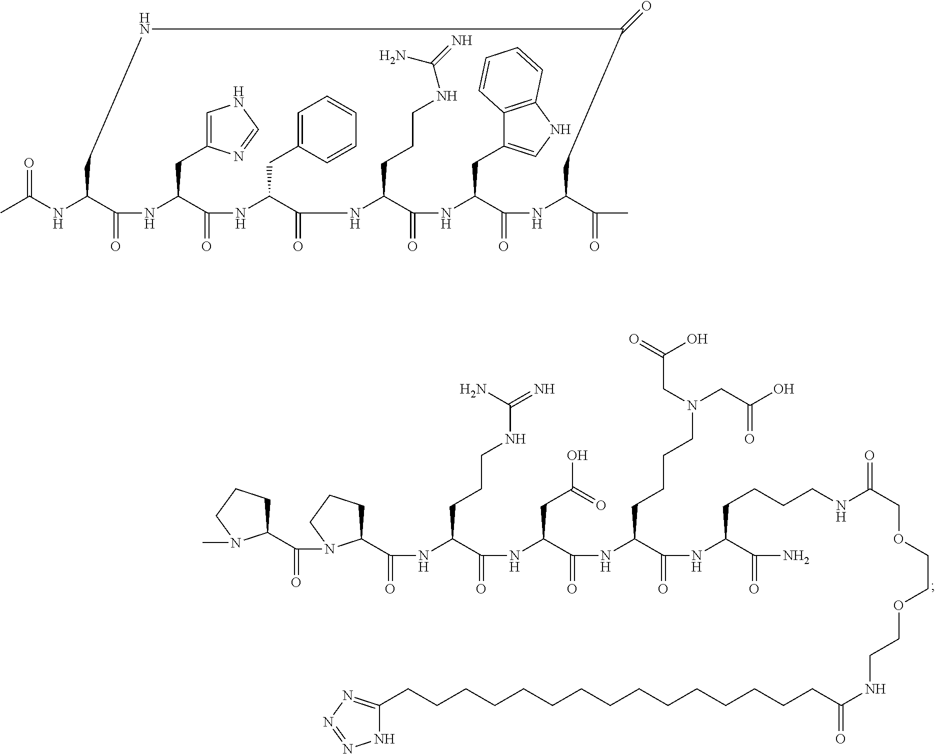 [SMILES: CC(=O)N[C@H]1CNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Glu-Gln-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

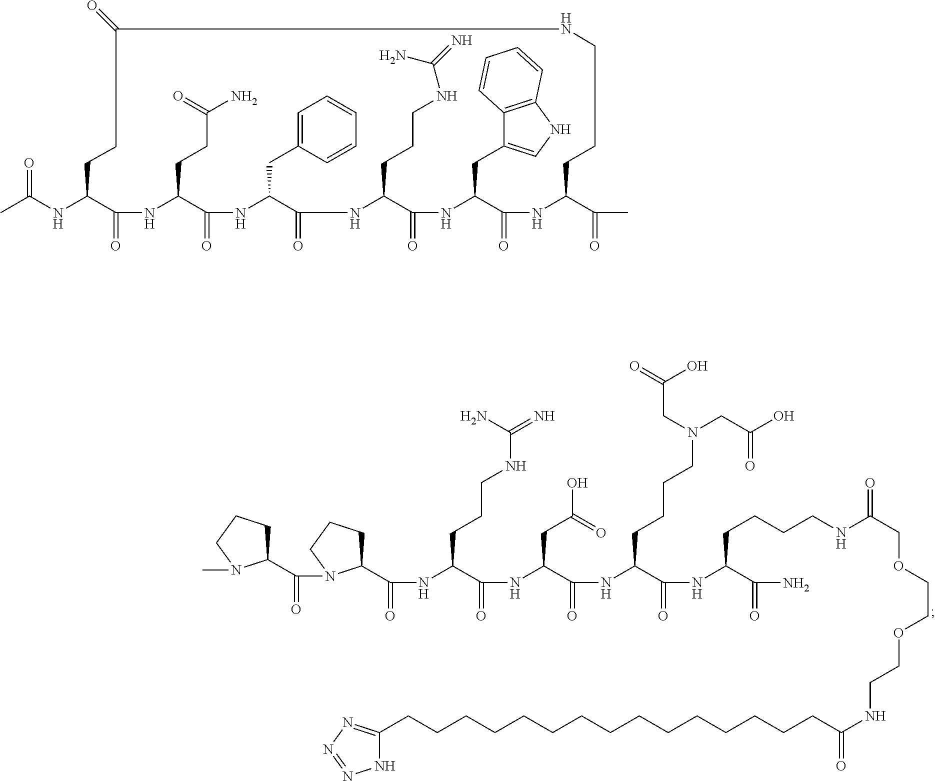

Ac-c[Glu-His-D-Phe-Arg-Trp-Orn]-Pro-Pro-Arg-Asp-Lys(biscarboxymethyl)-Lys({2-[2-(17-carboxyheptadecanoylamino)ethoxy]ethoxy}acetyl)-NH₂

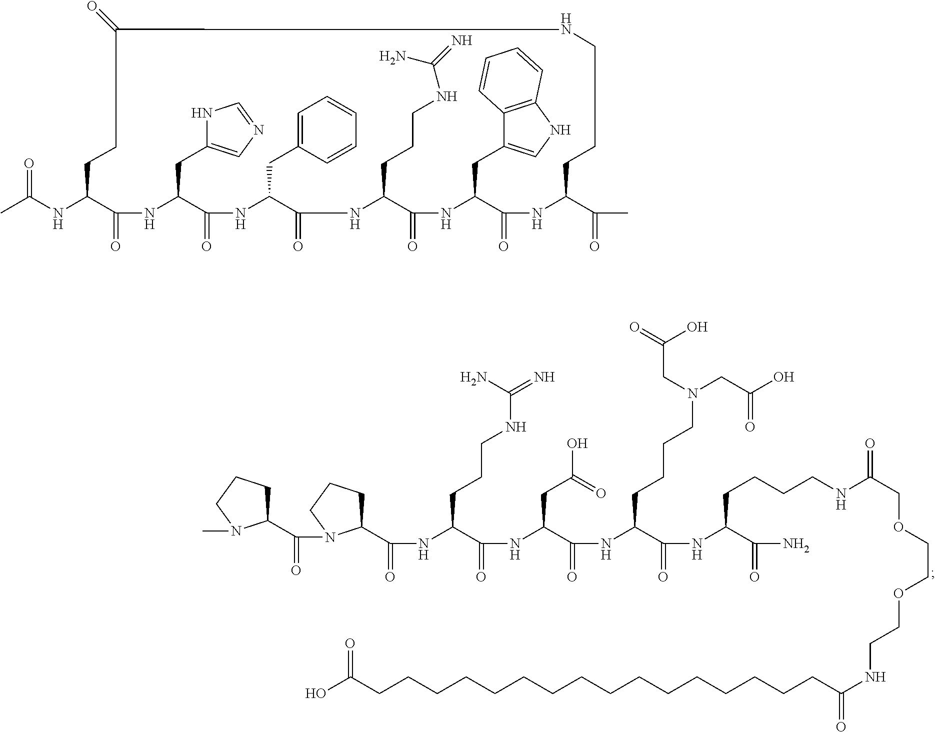

Ac-c[Orn-His-D-Phe-Arg-Trp-Glu]-Pro-Ser-Ser-Ser-Glu-Glu-Ser-Ser-Lys({2-[2-(16-carboxyhexadecanoylamino)ethoxy]ethoxy}acetyl)-NH₂

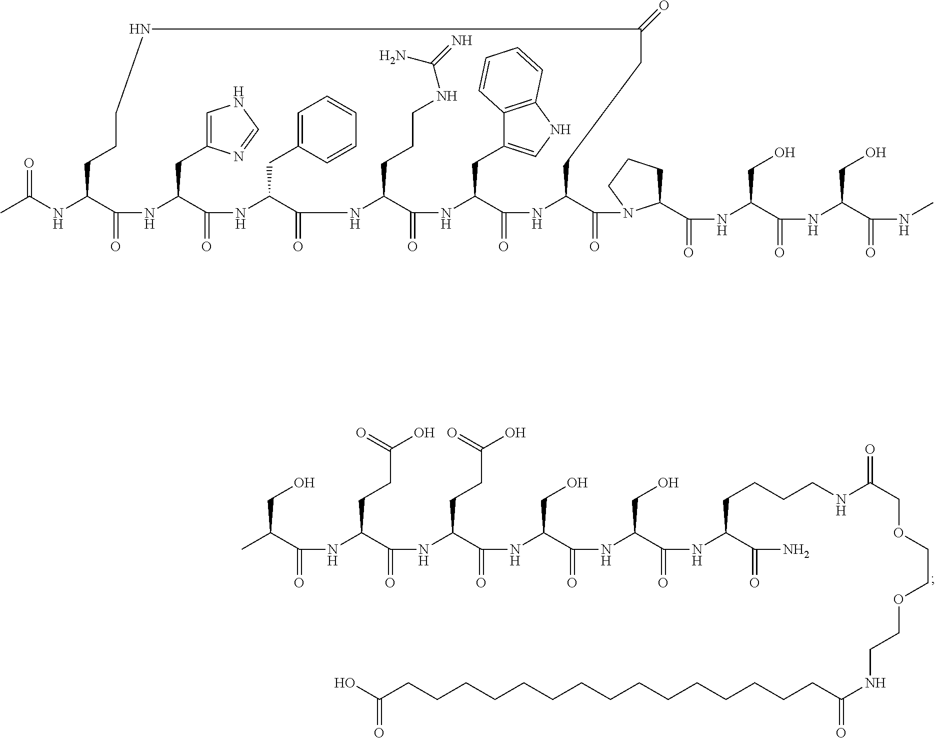

Ac-c[Orn-Glu-His-D-Phe-Arg-Trp-Gly-Asp]-Pro-Ser-Lys-Glu-Glu-Ser-Ser-Lys({2-[2-(16-carboxyhexadecanoylamino)ethoxy]ethoxy}acetyl)-NH₂

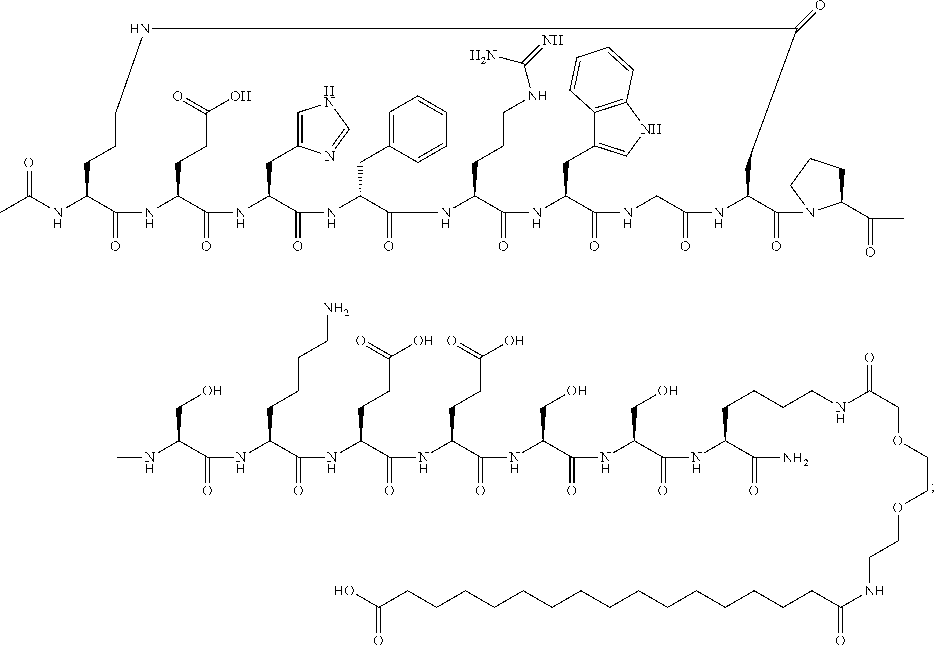

Ac-c[Asp-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

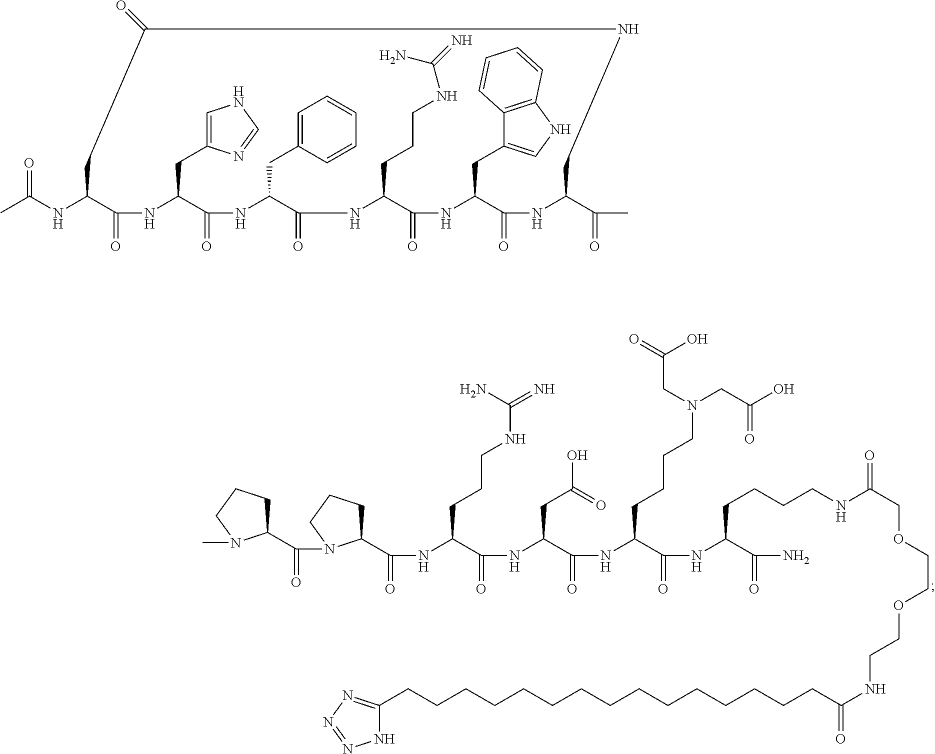

Ac-c[Orn-His-D-Phe-Arg-Trp-Asp]-Pro-Pro-Arg-Glu-Lys(biscarboxymethyl)-Lys({2-[2-(15-carboxypentadecanoylamino)ethoxy]ethoxy}acetyl)-NH₂

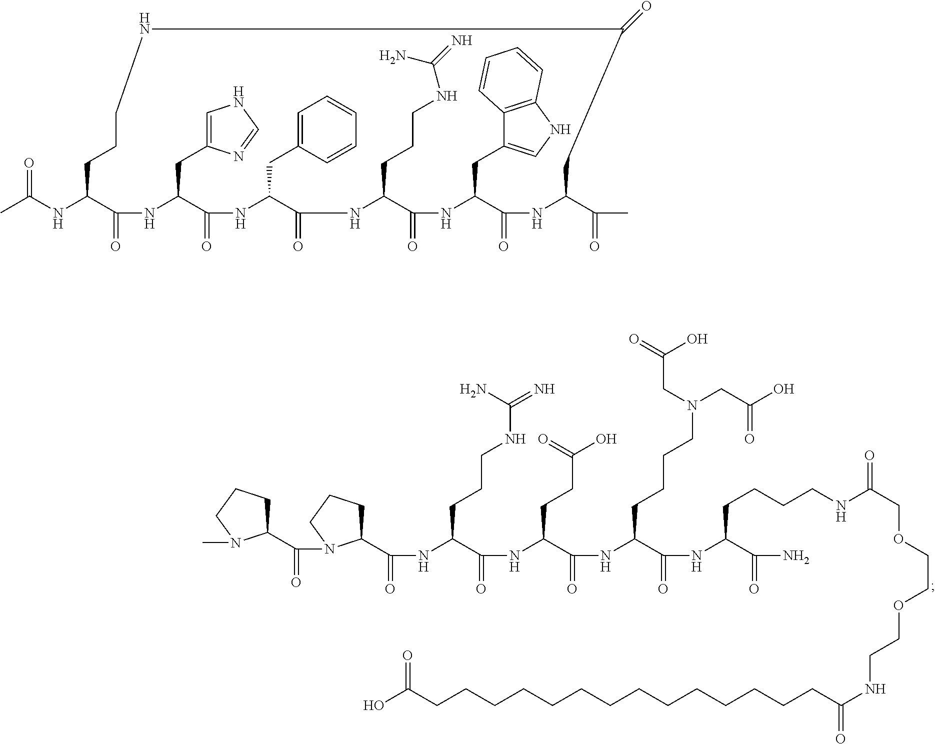 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

Ac-c[Glu-His-D-Phe-Arg-Trp-Orn]-Pro-Pro-His-Asp-Lys(biscarboxymethyl)-Lys[(2-{2-[16-(tetrazol-5-yl)hexadecanoylamino]ethoxy}ethoxy)acetyl]-NH₂

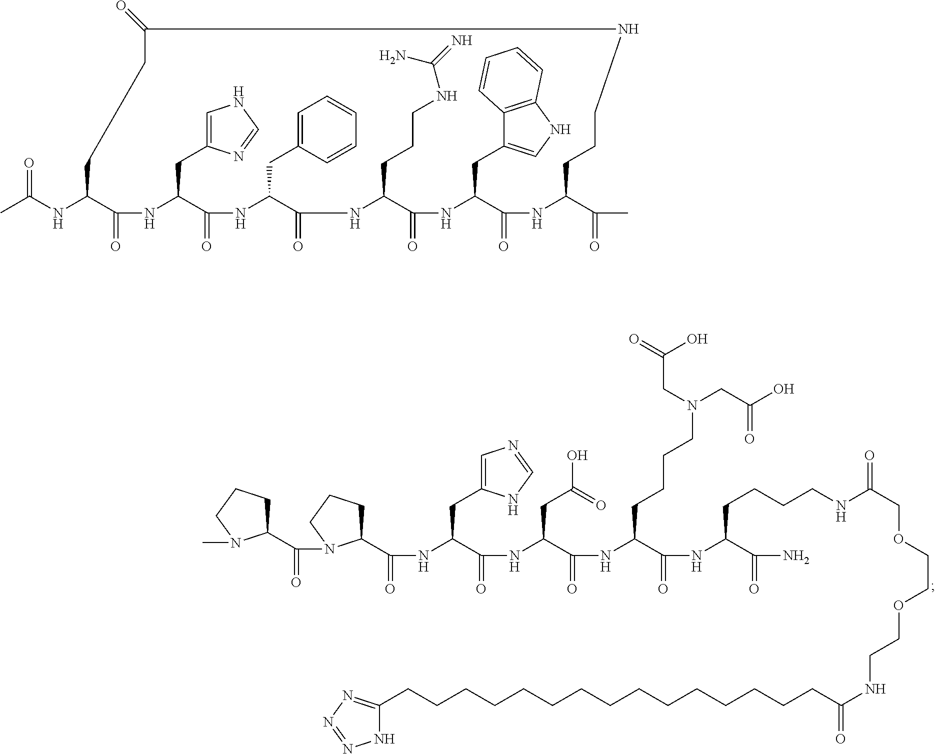

Ac-c[Lys-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂

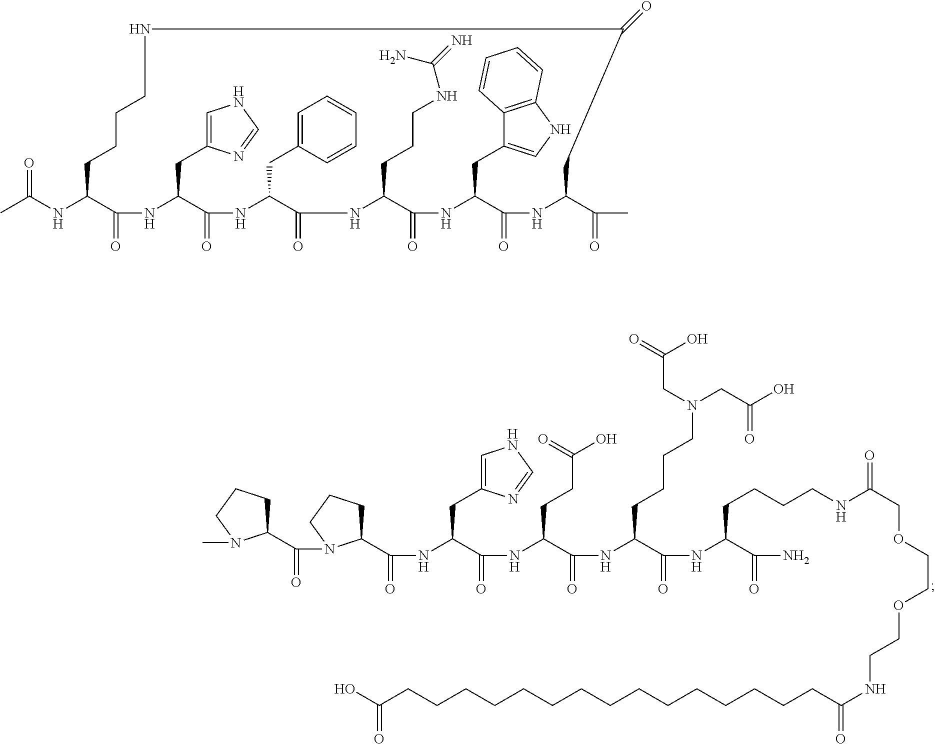 [SMILES: CC(=O)N[C@H]1CCCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]

Ac-c[Lys-His-D-Phe-Arg-Trp-Asp]-Pro-Pro-His-Ser-Lys(biscarboxymethyl)-Ser-Ser-Glu-Lys({2-[2-(16-carboxyhexadecanoylamino)ethoxy]ethoxy}acetyl)-NH₂

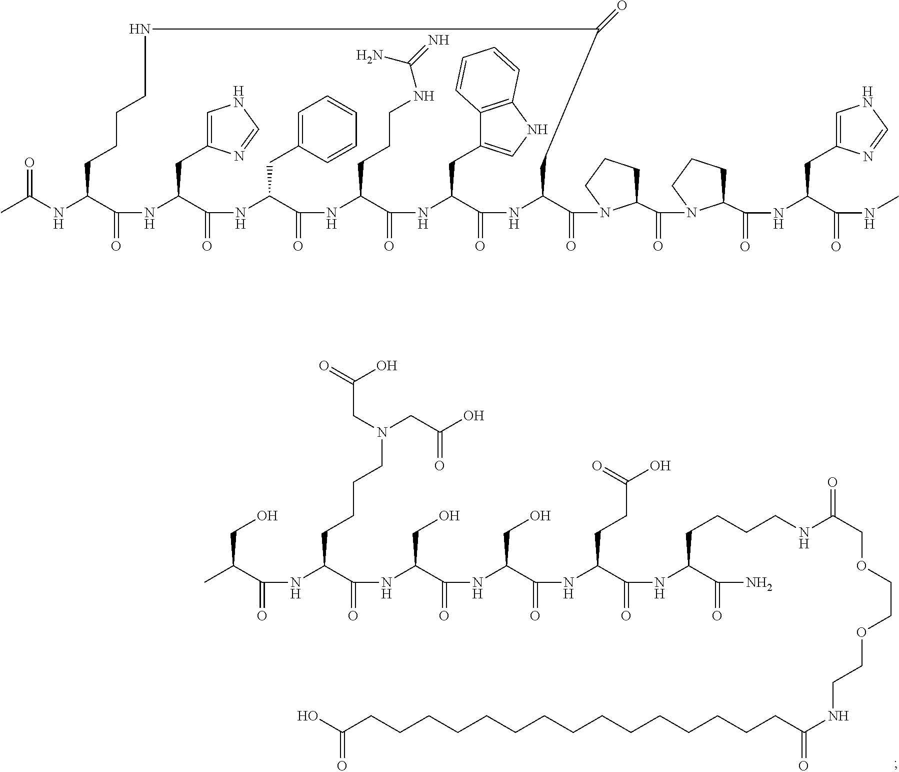

Ac-c[Orn-His-D-Phe-Arg-Trp-Asp]-Pro-Pro-His-Glu-Lys(biscarboxymethyl)-Lys({2-[2-(17-carboxyheptadecanoylamino)ethoxy]ethoxy}acetyl)-NH₂

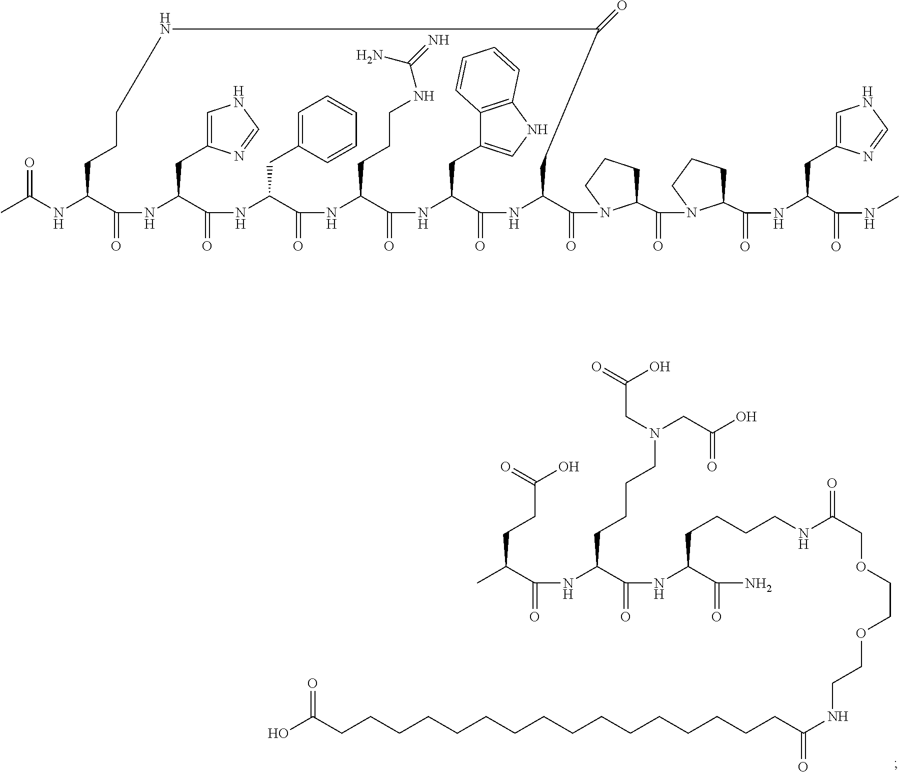 [SMILES: CNC(=O)[C@H](Cc1c[nH]cn1)NC(=O)[C@@H]1CCCN1C(=O)[C@@H]1CCCN1C(=O)[C@@H]1CC(=O)NCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]

Ac-c[Dap-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

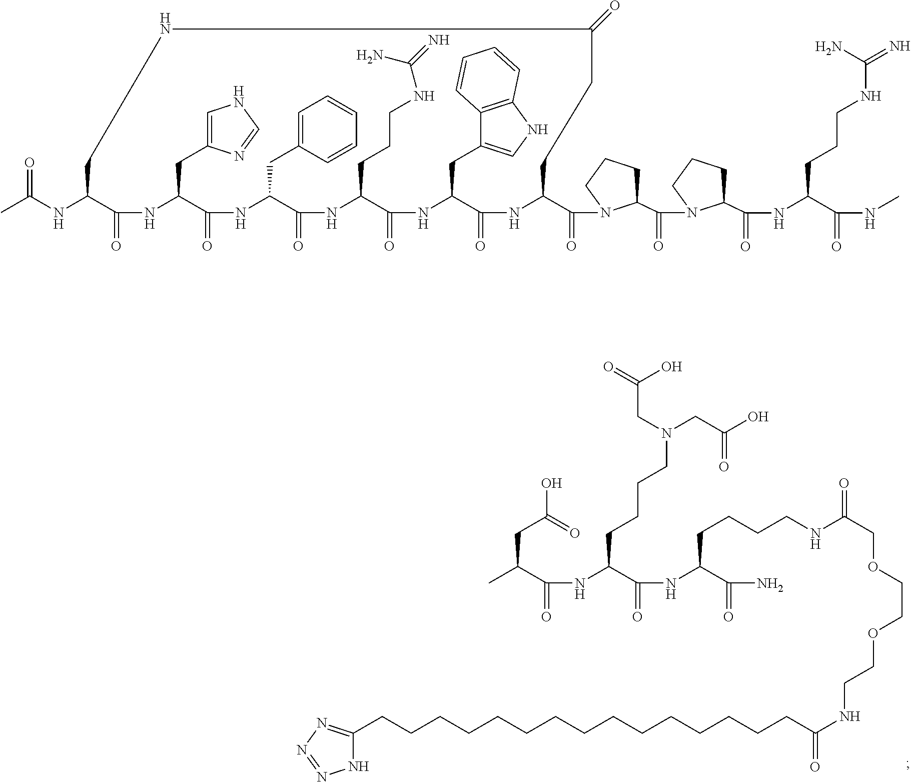 [SMILES: CNC(=O)[C@H](CCCNC(=N)N)NC(=O)[C@@H]1CCCN1C(=O)[C@@H]1CCCN1C(=O)[C@@H]1CCC(=O)NC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Glu-His-D-Phe-Arg-Trp-Lys]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

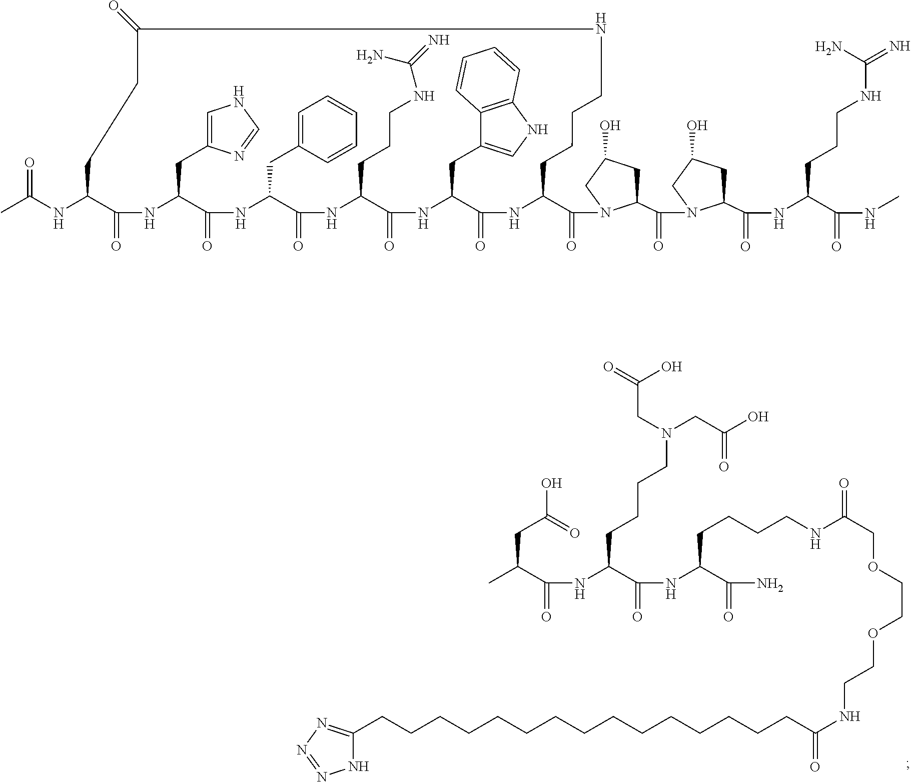 [SMILES: CNC(=O)[C@H](CCCNC(=N)N)NC(=O)[C@@H]1C[C@@H](O)CN1C(=O)[C@@H]1C[C@@H](O)CN1C(=O)[C@@H]1CCCCNC(=O)CC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Lys-His-D-Phe-Arg-Trp-Asp]-Pro-Ser-His-Glu-Lys(biscarboxymethyl)-Lys({2-[2-(16-carboxyhexadecanoylamino)ethoxy]ethoxy}acetyl)-NH₂

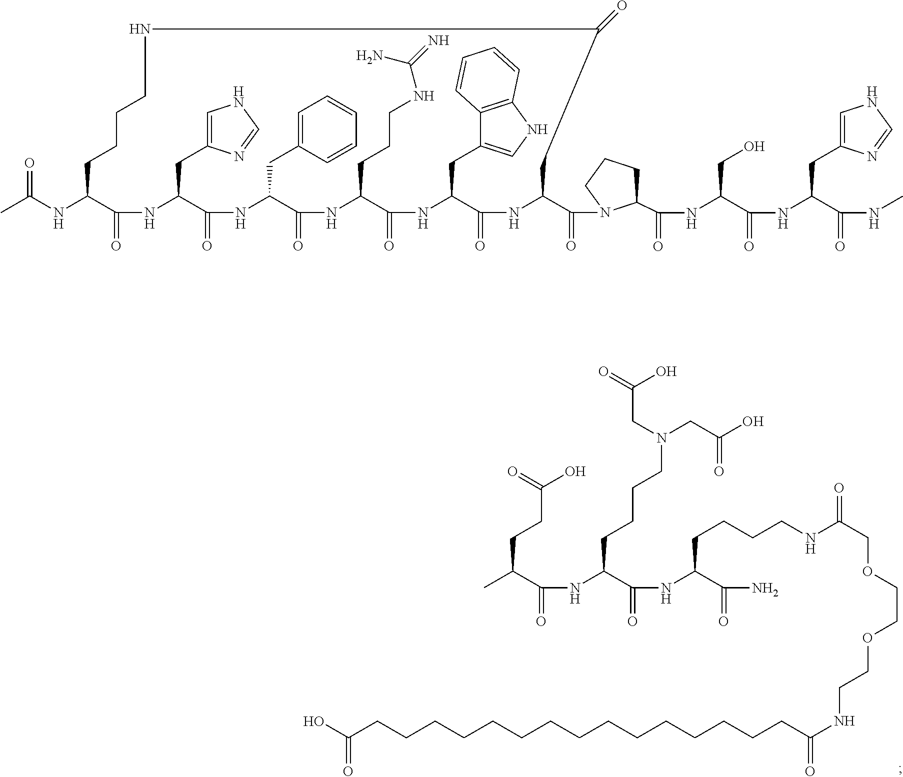 [SMILES: CNC(=O)[C@H](Cc1c[nH]cn1)NC(=O)[C@H](CO)NC(=O)[C@@H]1CCCN1C(=O)[C@@H]1CC(=O)NCCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]

Ac-c[Orn-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl)

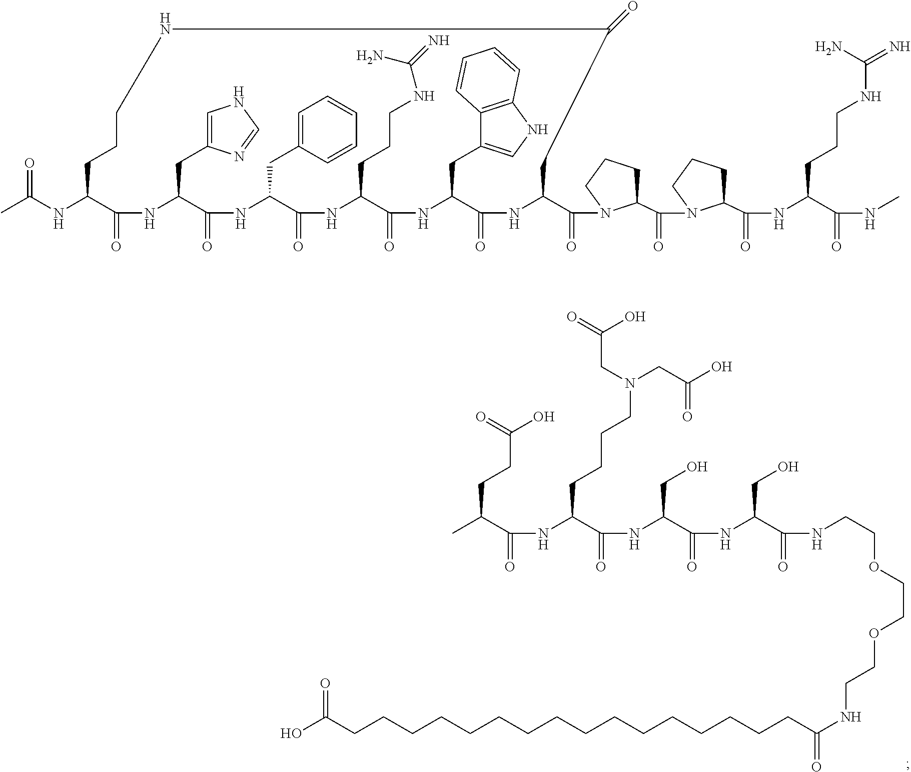 [SMILES: CNC(=O)[C@H](CCCNC(=N)N)NC(=O)[C@@H]1CCCN1C(=O)[C@@H]1CCCN1C(=O)[C@@H]1CC(=O)NCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]

Ac-c[Orn-His-D-Phe-Arg-Trp-Asp]-Pro-Pro-His-Glu-Lys(biscarboxymethyl)-Lys({2-[2-(15-carboxypentadecanoylamino)ethoxy]ethoxy}acetyl)-NH₂

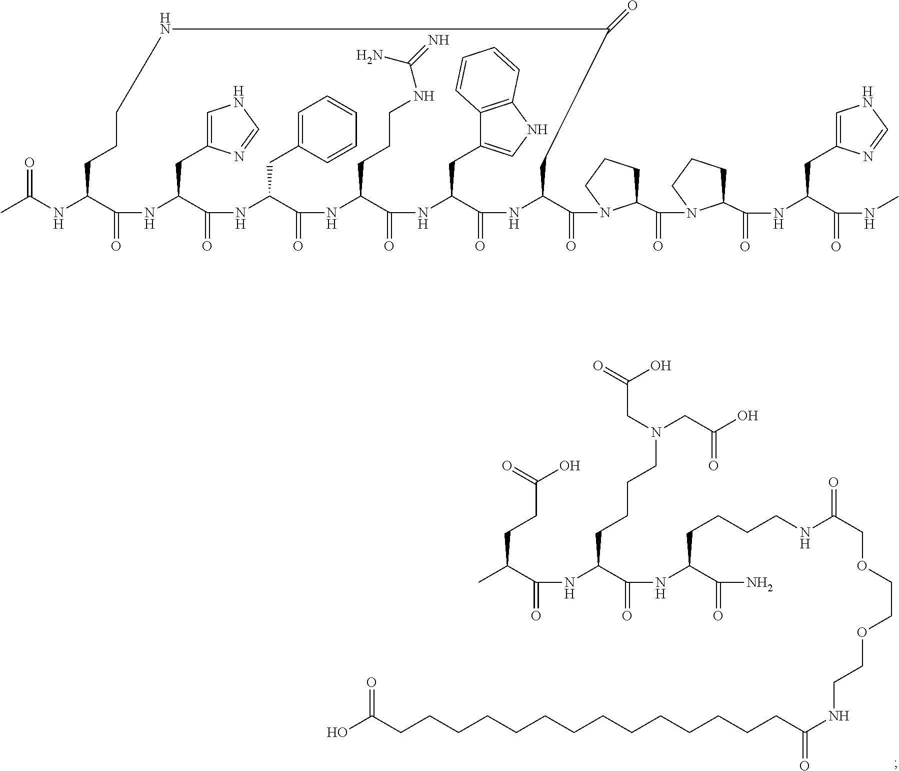 [SMILES: CNC(=O)[C@H](Cc1c[nH]cn1)NC(=O)[C@@H]1CCCN1C(=O)[C@@H]1CCCN1C(=O)[C@@H]1CC(=O)NCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

Ac-c[Lys-His-D-Phe-Arg-Trp-Asp]-Pro-Pro-His-Glu-Glu-Ser-Ser-Glu-Lys({2-[2-(16-carboxyhexadecanoylamino)ethoxy]ethoxy}acetyl)-NH₂

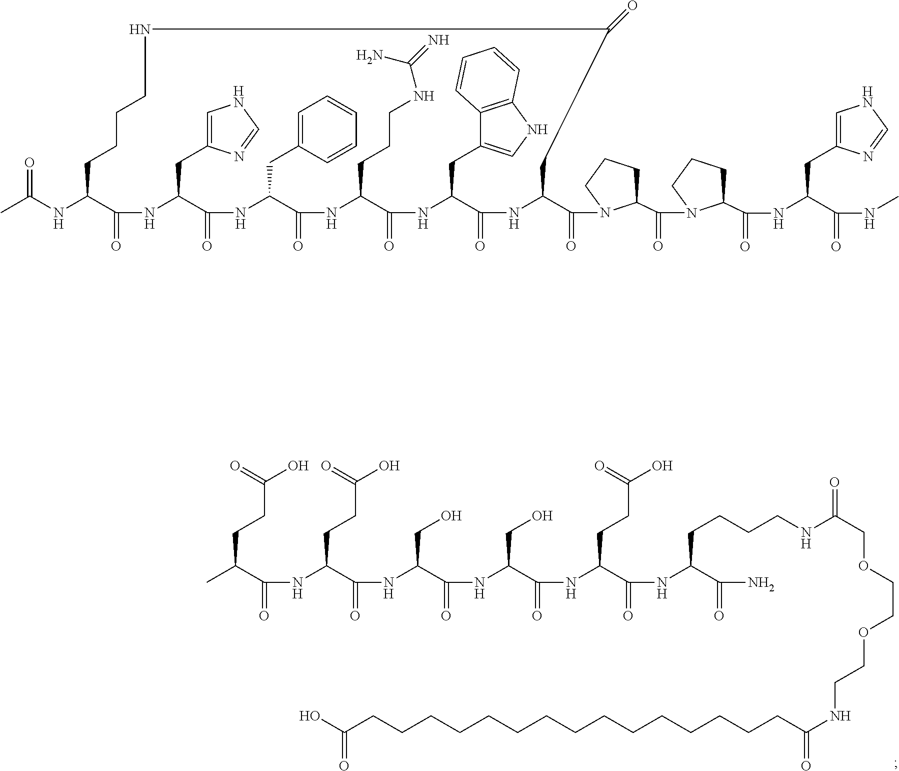

Ac-c[Lys-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl)

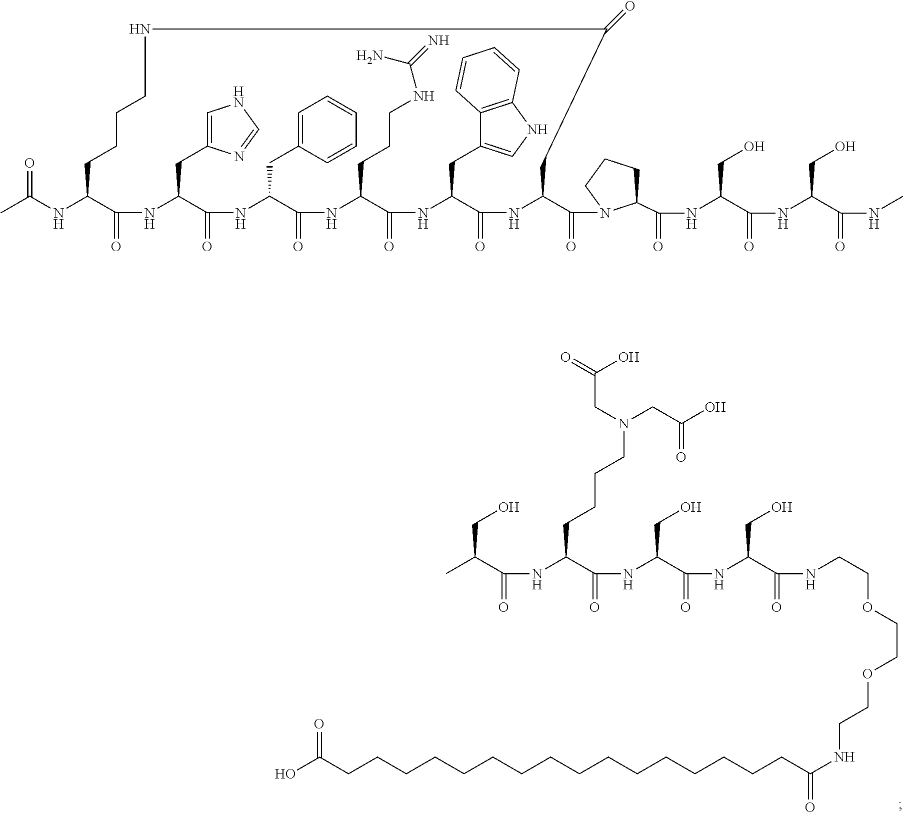 [SMILES: CNC(=O)[C@H](CO)NC(=O)[C@H](CO)NC(=O)[C@@H]1CCCN1C(=O)[C@@H]1CC(=O)NCCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CO)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]

Ac-c[Glu-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

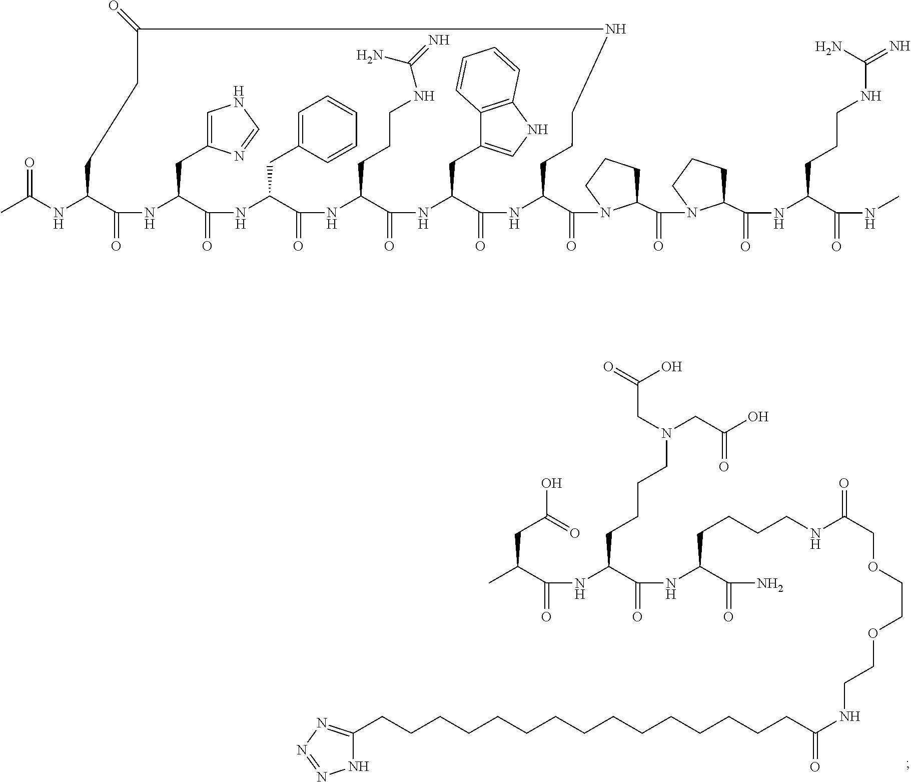 [SMILES: CNC(=O)[C@H](CCCNC(=N)N)NC(=O)[C@@H]1CCCN1C(=O)[C@@H]1CCCN1C(=O)[C@@H]1CCCNC(=O)CC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Orn-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂

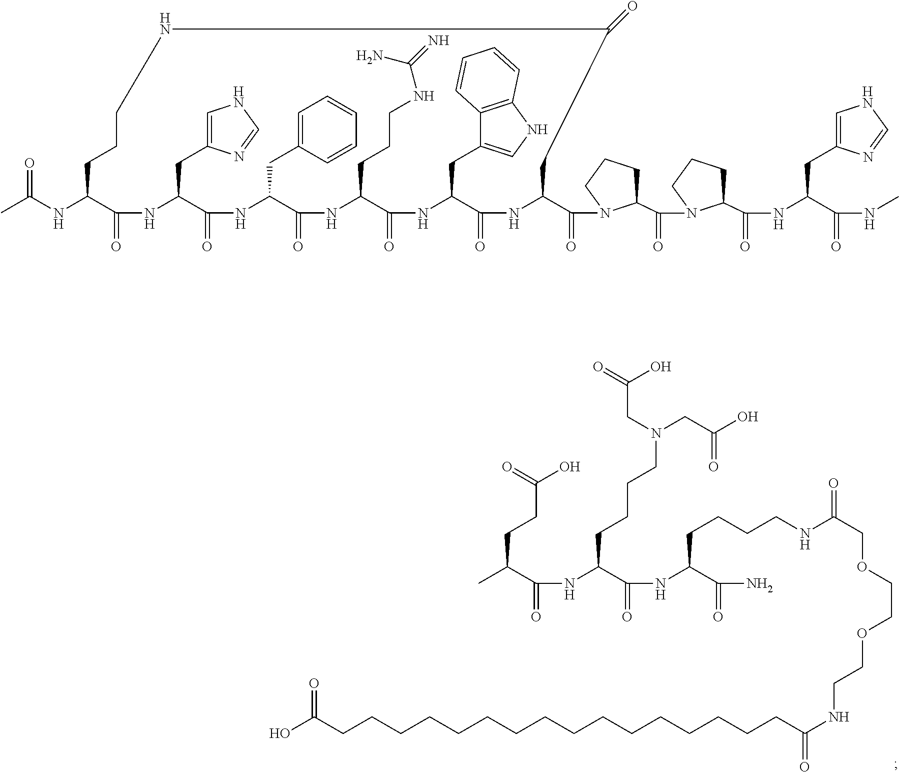 [SMILES: CNC(=O)[C@H](Cc1c[nH]cn1)NC(=O)[C@@H]1CCCN1C(=O)[C@@H]1CCCN1C(=O)[C@@H]1CC(=O)NCCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]

Ac-c[Glu-His-D-Phe-Arg-Trp-Orn]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

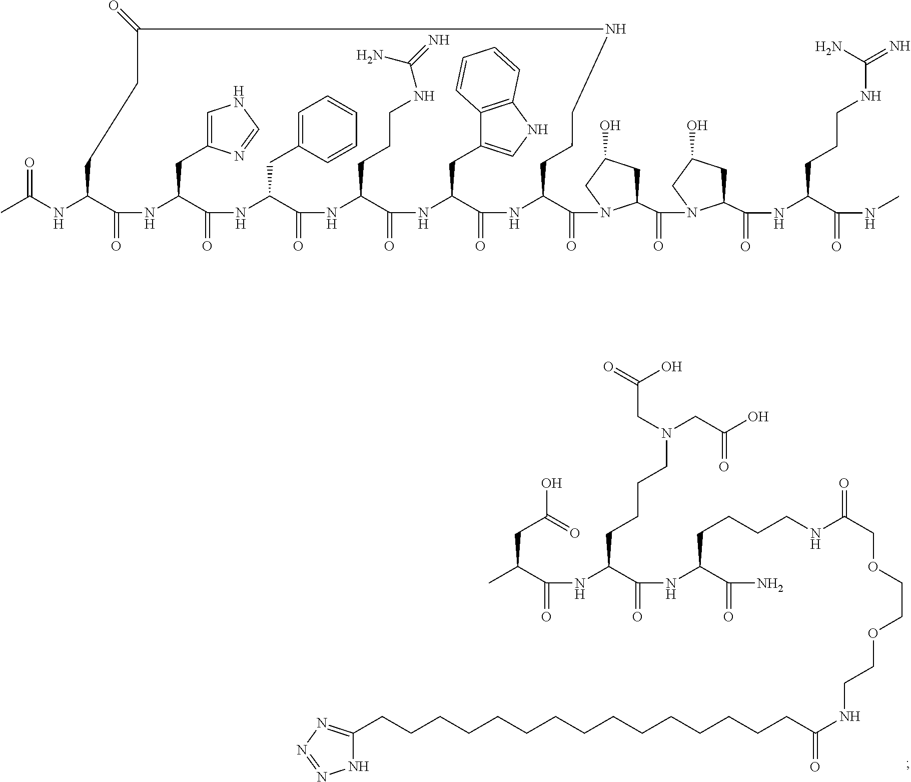 [SMILES: CNC(=O)[C@H](CCCNC(=N)N)NC(=O)[C@@H]1C[C@@H](O)CN1C(=O)[C@@H]1C[C@@H](O)CN1C(=O)[C@@H]1CCCNC(=O)CC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Orn-His-D-Phe-Arg-Trp-Glu]-Pro-Ser-Ser-Ser-Lys(biscarboxymethyl)-Ser-Ser-Lys({2-[2-(16-carboxyhexadecanoylamino)ethoxy]ethoxy}acetyl)-NH₂

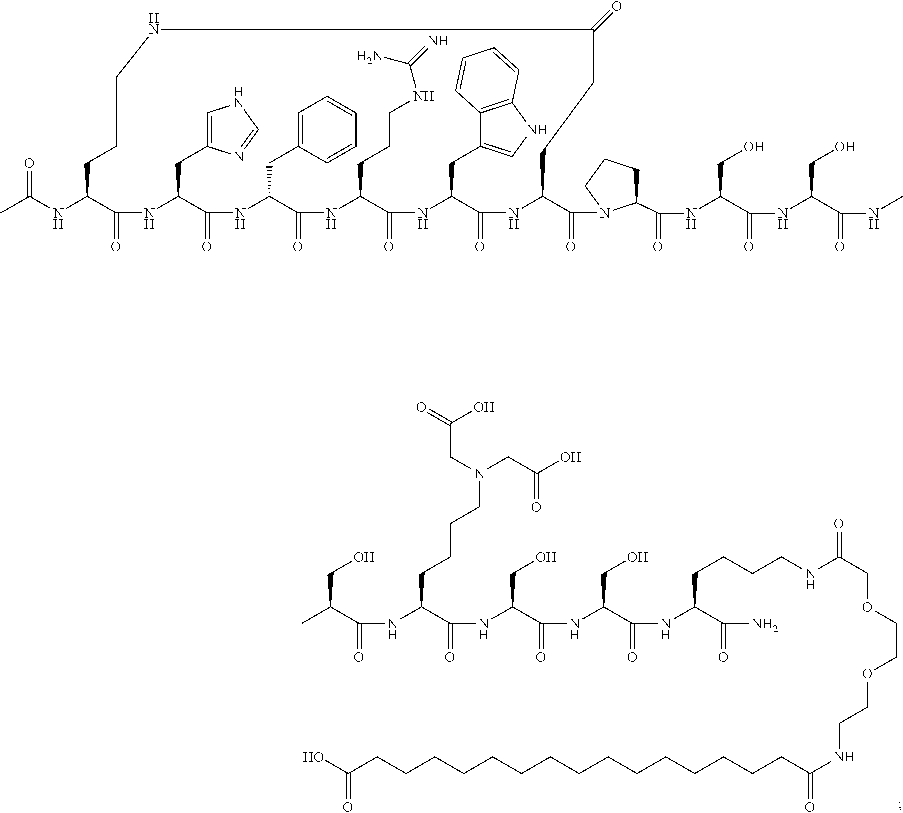

Ac-c[homoCys-Ser-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂

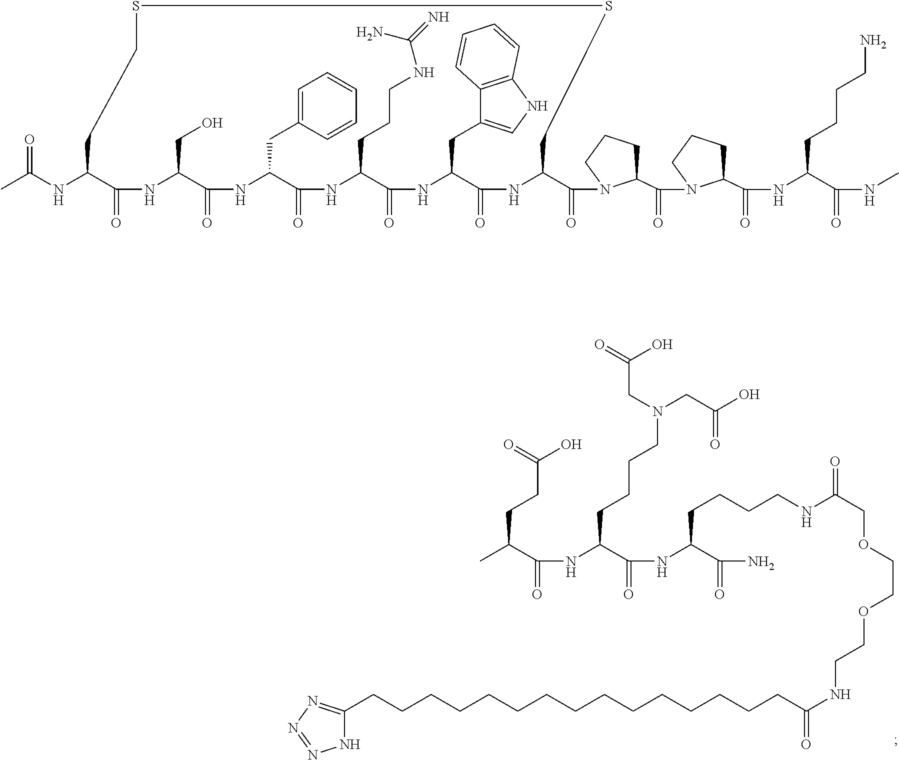 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](CO)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

Ac-c[Cys-Glu-His-D-Phe-Arg-Trp-Gly-Cys]-Pro-Pro-Lys-Glu-Lys(biscarboxymethyl)-Lys({2-[2-(15-carboxypentadecanoylamino)ethoxy]ethoxy}acetyl)-NH₂

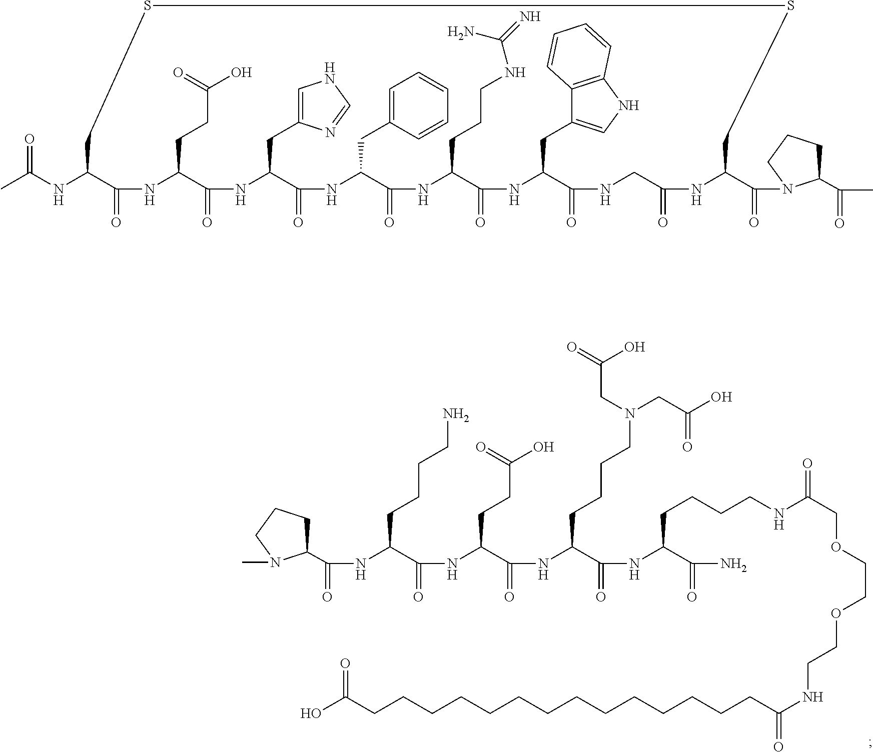

Ac-c[homoCys-His-D-Phe-Arg-Trp-Cys]-Ser-Pro-Lys-Glu-Lys(biscarboxymethyl)-Lys({2-[2-(15-carboxypentadecanoylamino)ethoxy]ethoxy}acetyl)-NH₂

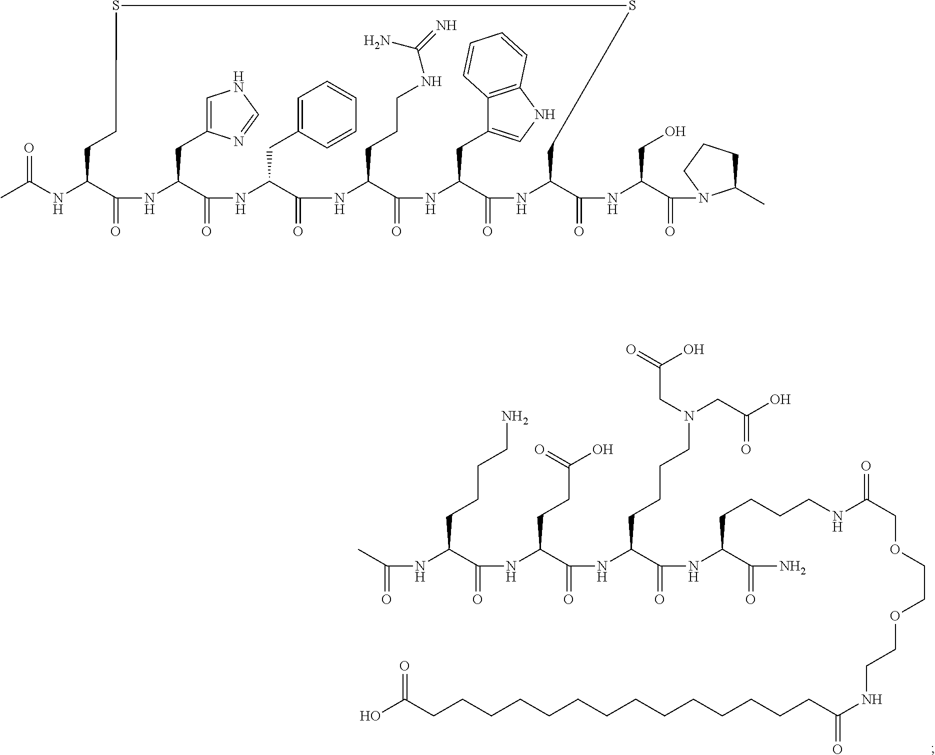

Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

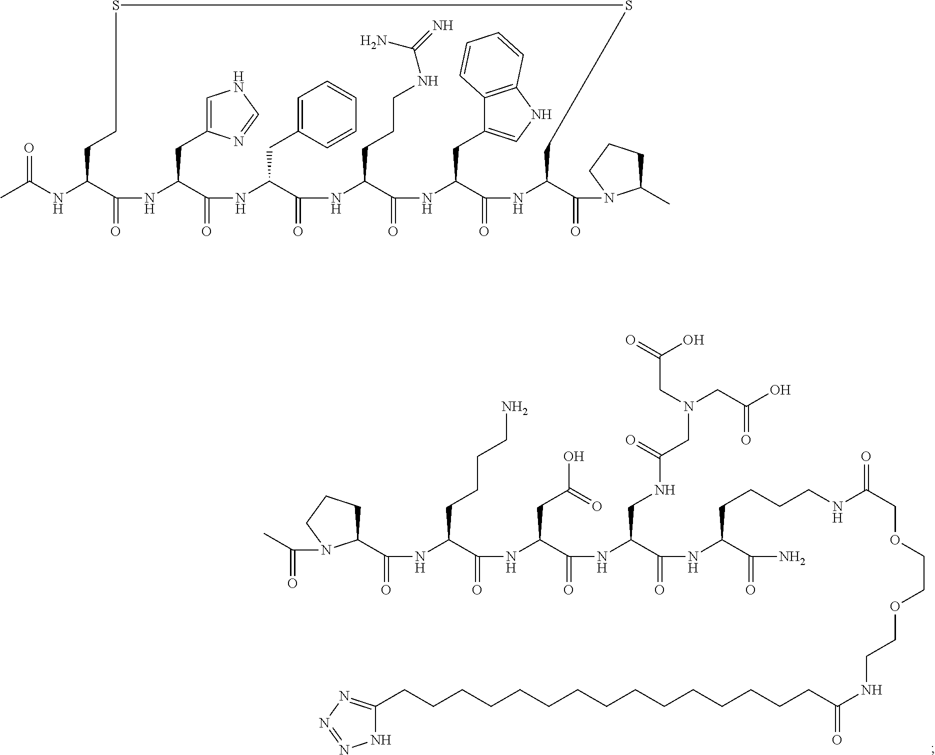

Ac-c[homoCys-His-D-Phe-Arg-Trp-Cys]-Ser-Pro-His-Glu-Lys(biscarboxymethyl)-Lys({2-[2-(17-carboxyheptadecanoylamino)ethoxy]ethoxy}acetyl)-NH₂

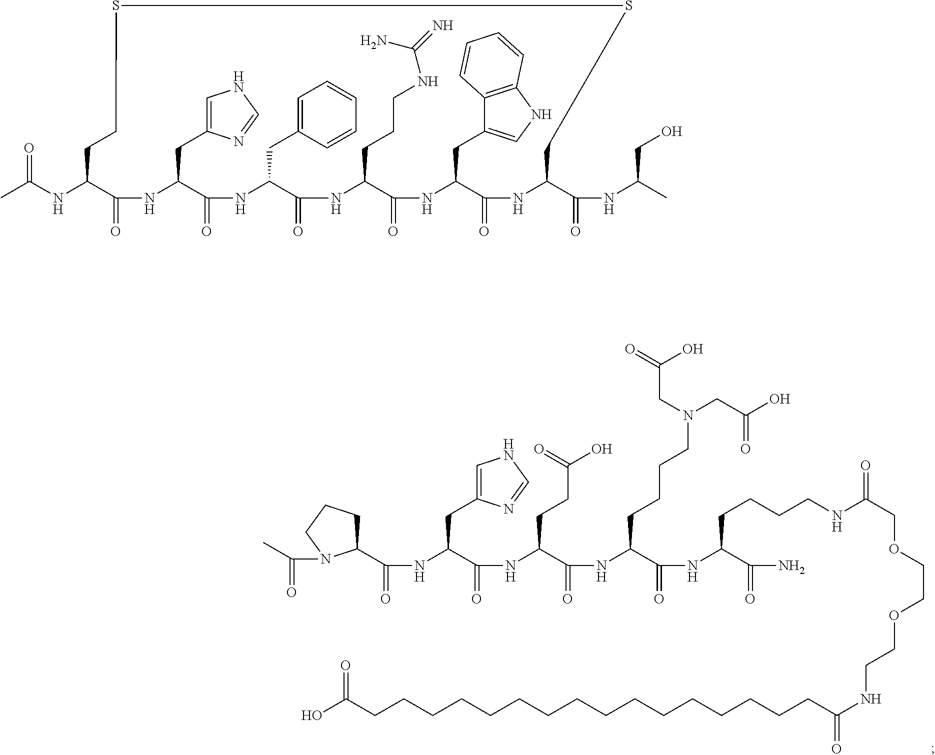 [SMILES: CC(=O)N[C@H]1CCSSC[C@@H](C(=O)N[C@H](C)CO)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O ; ";"]

Ac-c[Cys-Glu-His-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂

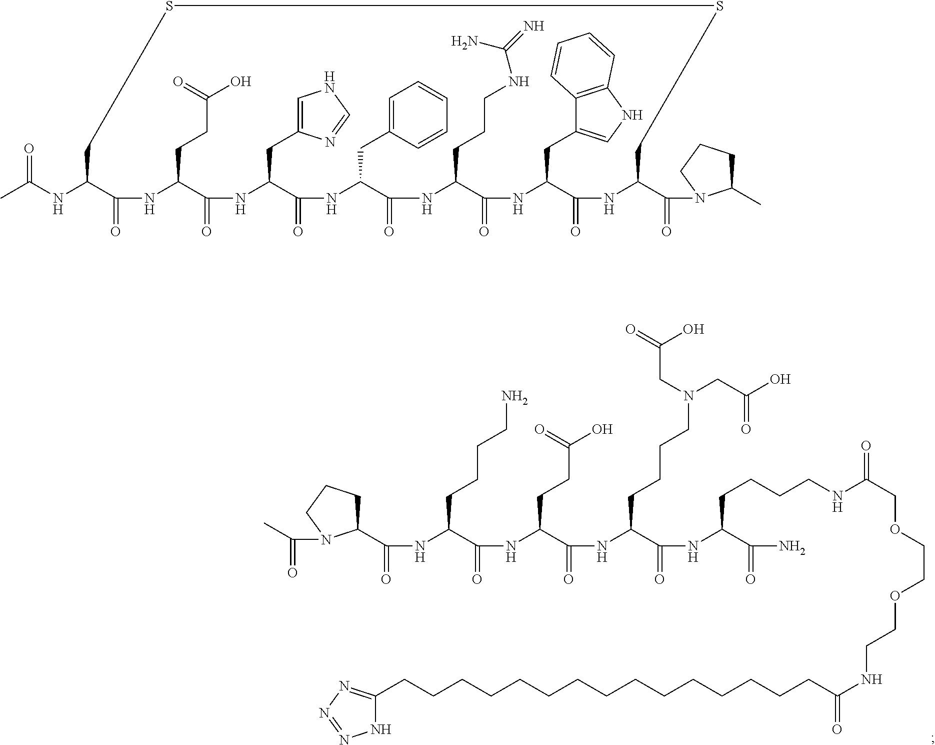

Ac-c[homoCys-His-D-Phe-Arg-Trp-Cys]-Pro-Pro-Lys-Glu-Dap(BCMA)-Lys[(2-{2-[16-(tetrazol-5-yl)hexadecanoylamino]ethoxy}ethoxy)acetyl]-NH₂

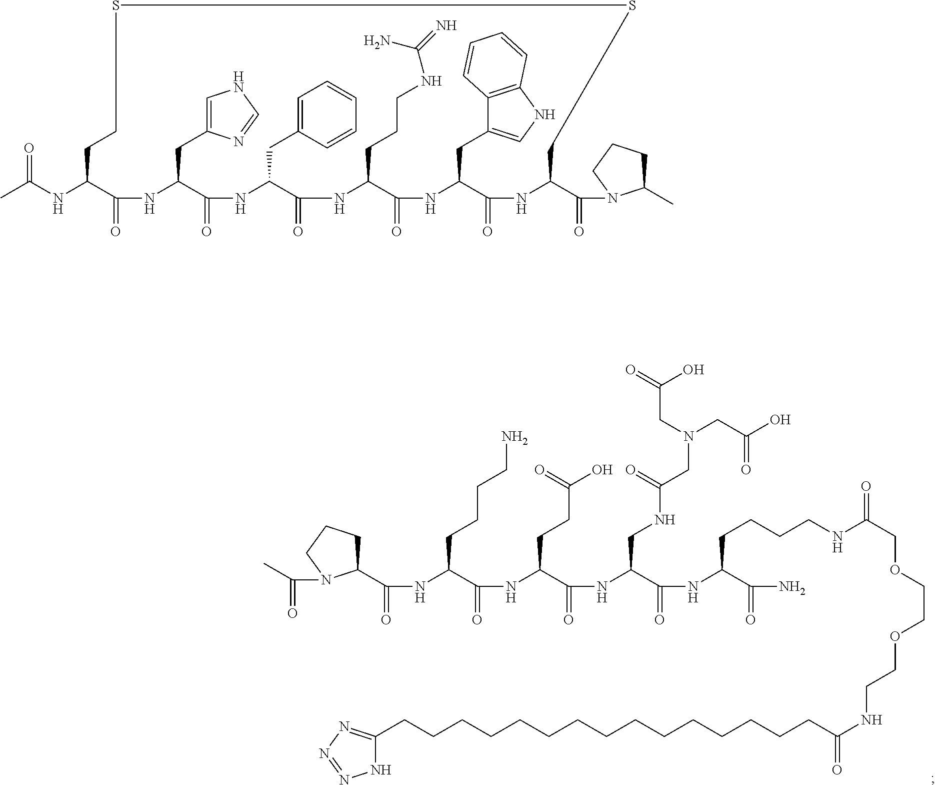

Ac-c[homoCys-His-D-Phe-Arg-Trp-Cys]-Pro-Pro-Lys-Asp-Lys(BCMA)-Lys[(2-{2-[16-(tetrazol-5-yl)hexadecanoylamino]ethoxy}ethoxy)acetyl]-NH₂

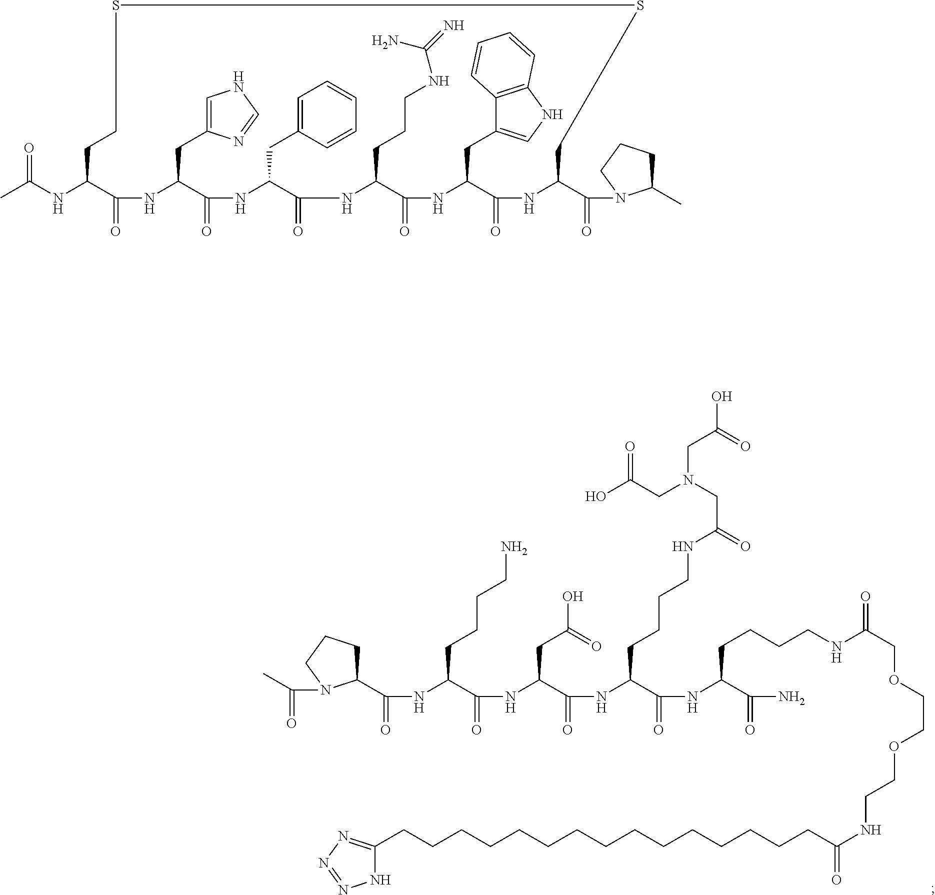

Ac-c[homoCys-Gln-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

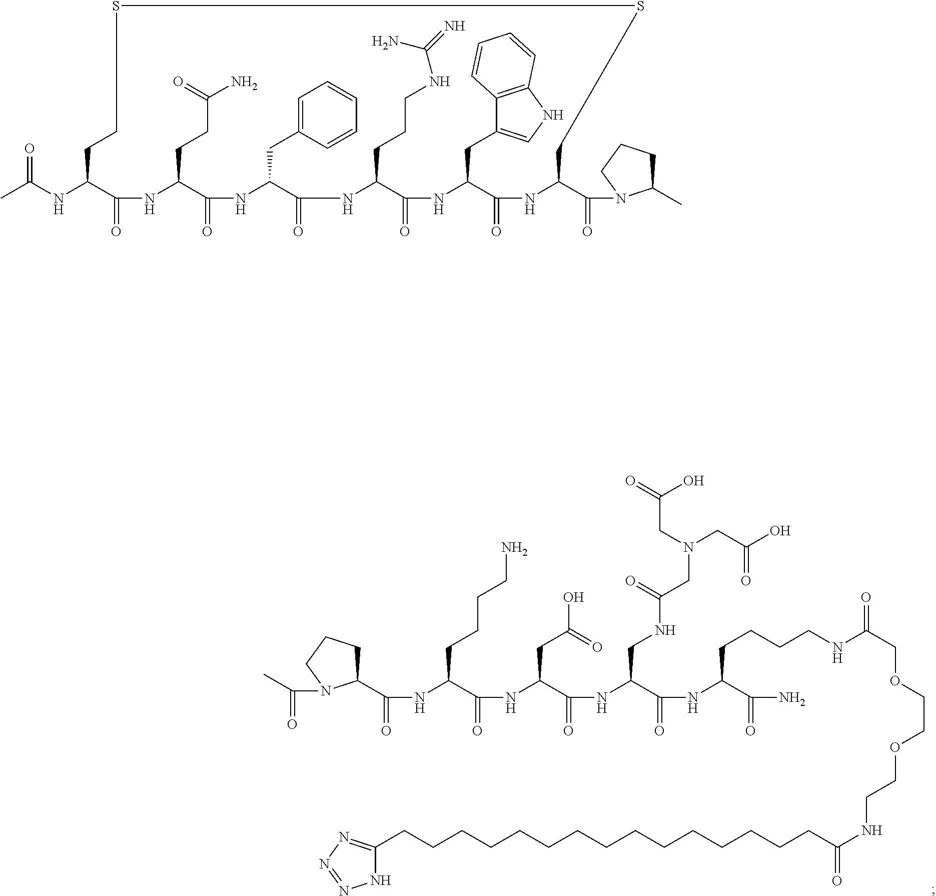

Ac-c[homoCys-Hyp-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

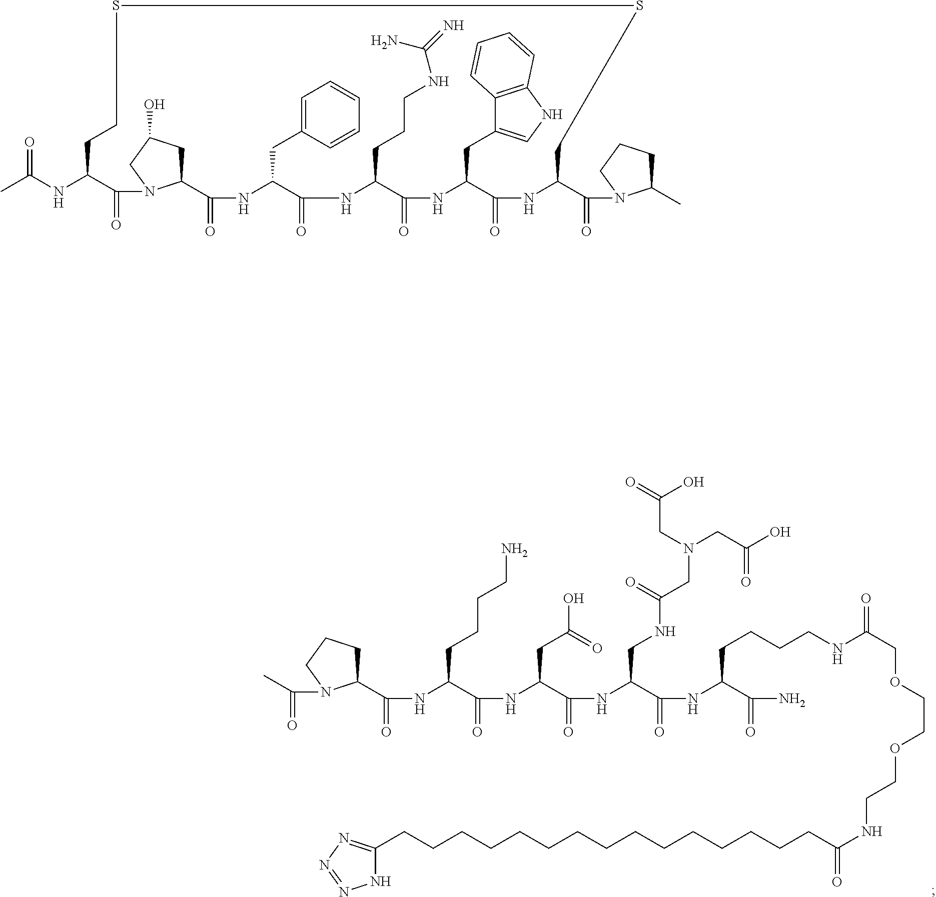

Ac-c[Cys-Glu-His-D-Phe-Arg-Trp-Gly-Cys]-Pro-Pro-Lys-Glu-Glu-Lys({2-[2-(15-carboxypentadecanoylamino)ethoxy]ethoxy}acetyl)-NH₂

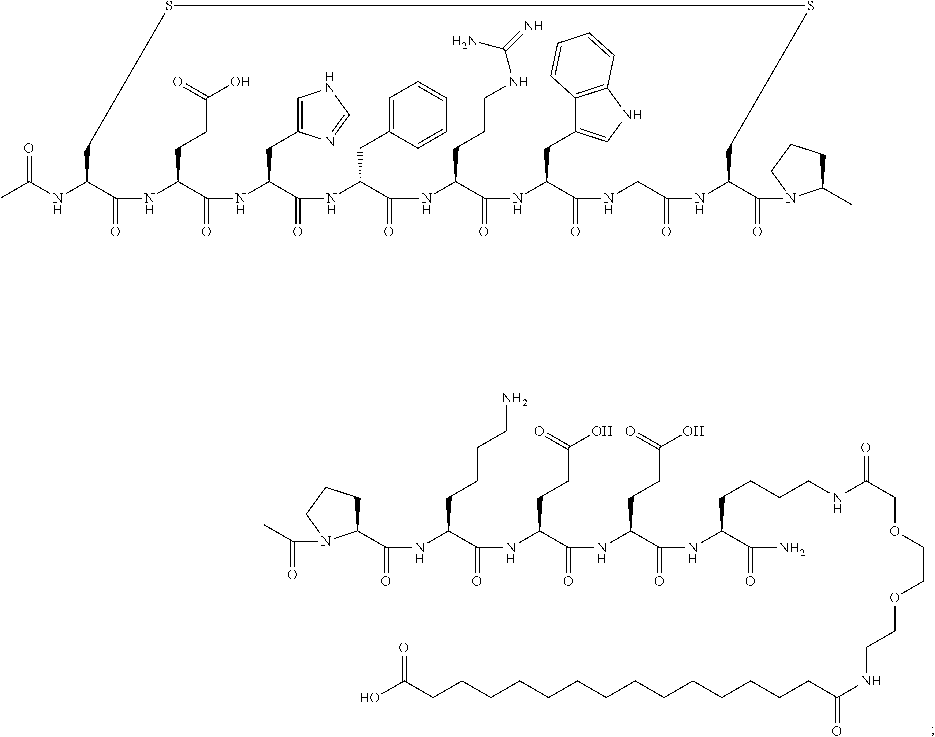

Ac-c[homoCys-His-D-Phe-Arg-Trp-Cys]-Pro-Pro-Lys-Asp-Dap(BCMA)-Lys({2-[2-(13-carboxytridecanoylamino)ethoxy]ethoxy}acetyl)-NH₂

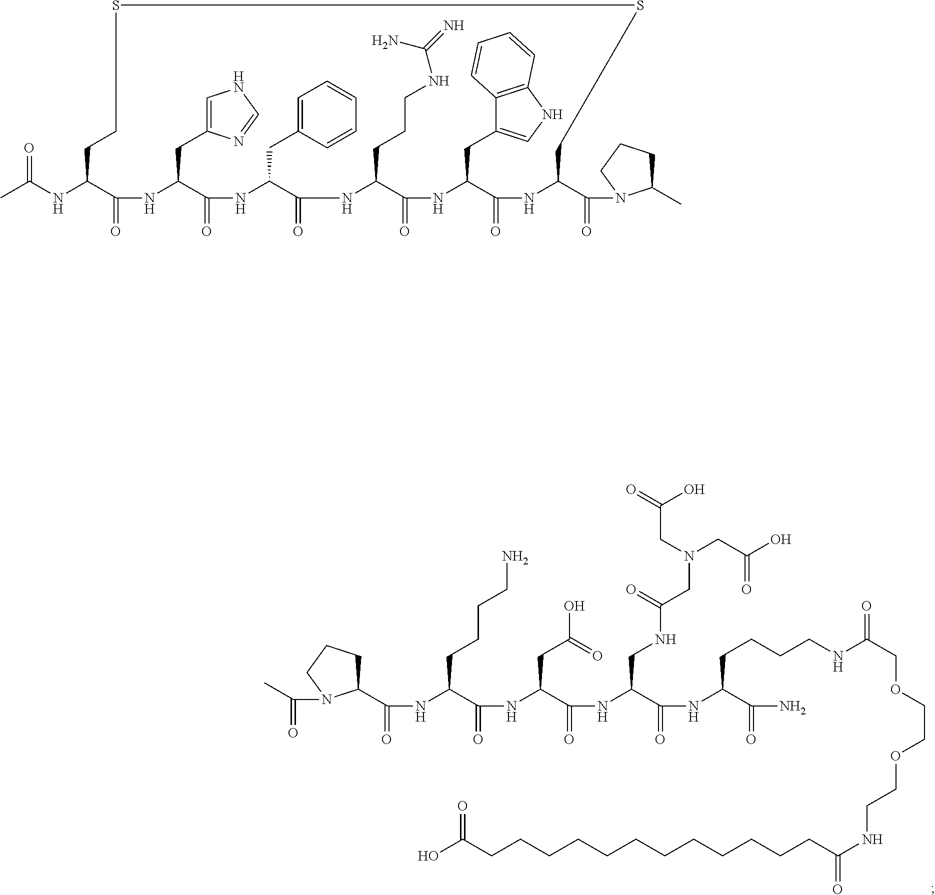

and
Ac-c[homoCys-His-D-Phe-Arg-Trp-Cys]-Pro-Pro-Ser-Glu-Lys(biscarboxymethyl)-Lys({2-[2-(15-carboxypentadecanoylamino)ethoxy]ethoxy}acetyl)-NH₂

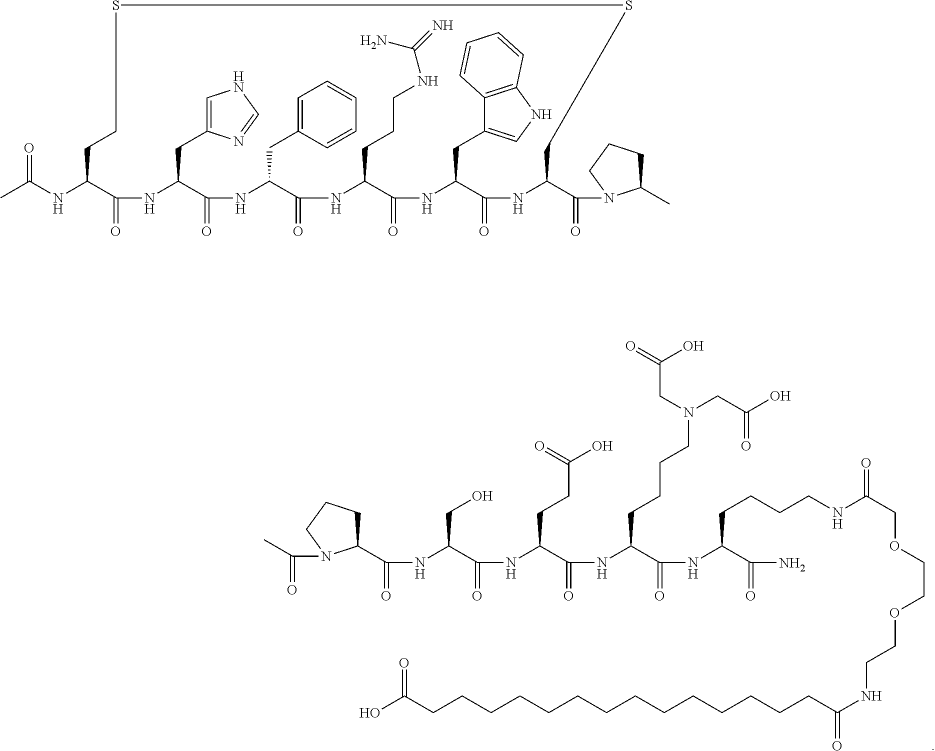

101. A compound according to any of embodiments 1-100, which is soluble in an aqueous solution at neutral to weakly basic pH.
102. A compound according to any of embodiments 1-100, which is soluble in an aqueous solution at pH 7.0 to 8.0.
103. A compound according to any of embodiments 1-100, which is soluble in an aqueous solution at a pH about 7.5.

The present invention also encompasses combinations of two or more embodiments of compounds of the invention as outlined above.

In one aspect of the present invention, the compound of the invention is an agonist of a melanocortin receptor, notably an agonist of MC4. In another aspect of the invention, the compound is a selective agonist of MC4. In this context, selectivity is to be understood in relation to the activity of the compound with respect to MC1, MC3 and/or MC5. If a compound is a significantly more potent as a MC4 agonist than as a MC1, MC3 and/or MC5 agonist, it is deemed to be a selective MC4 agonist. The binding affinity of a compound with respect to MC1, MC3, MC5 and MC4 may be determined by comparing the Ki from an MC1, MC3 or MC5 binding assay as described below under “Assay IV” (MC1), “Assay VIII” (MC3) and “Assay IX” (MC5), respectively, with Ki from an MC4 binding assay as described below under “Assay V” (MC4). If the binding affinity of a compound is more than 10 times, such as more than 50 times, e.g. more than 100 times greater ( ) with respect to MC4 than with respect to MC1, it is deemed to be a selective MC4 agonist with respect to MC1. If the binding affinity of a compound is more than 10 times, such as more than 50 times, e.g. more than 100 times greater (higher) with respect to MC4 than with respect to MC3, it is deemed to be a selective MC4 agonist with respect to MC3. If the binding affinity of a compound is more than 10 times, such as more than 50 times, e.g. more than 100 times greater with respect to MC4 than with respect to MC5, it is deemed to be a selective MC4 agonist with respect to MC5. The agonistic potency of a compound with respect to MC3, MC4 and MC5 may be determined in functional assays as described in “Assay II” (MC3 and MC5), “Assay X” (MC3) and “Assay III” (MC4). If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. In a particular aspect, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In a further aspect of the present invention, the compound of the present invention is both a selective MC3 agonist and a selective MC4 agonist. In this context, a compound is deemed to be a selective MC3 and MC4 agonist if it is significantly more potent as an agonist towards MC3 and MC4 than as an agonist toward MC1 and MC5. The selectivity of a compound with respect to MC1 and MC3 may be determined by comparing the binding affinity determined for MC1 as described in “Assay IV” with the binding affinity for MC3 determined as described in “Assay VIII”. If the binding affinity of a compound is more than 10 times, such as more than 50 times, e.g. more than 100 times greater with respect to MC3 than with respect to MC1, it is deemed to be a selective MC3 agonist with respect to MC1. The selectivity of a compound with respect to MC3 and MC5 may be determined by comparing the affinity determined as described in “Assay VIII and IX”. If the binding affinity of a compound is more than 10 times, such as more the 50 times, e.g. more than 100 times greater with respect to MC3 than with respect to MC5, it is deemed to be a selective MC3 agonist with respect to MC5. The MC4 selectivity of a compound with respect to MC3 and MC5 is determined as discussed above.

Compounds of the present invention may exert a protracted effect, i.e. the period of time in which they exert a biological activity is prolonged. Effect is defined as being protracted when a compound significantly reduces food intake in the period from 24 hours to 48 hours in test animals compared to the food intake in the same time period in the vehicle-treated control group of animals in “Assay I”. The protracted effect can be evaluated through different binding assays, for example the protracting effect may be evaluated in an indirect albumin-binding assay, in which Ki determined for binding in the presence of ovalbumin is compared with the EC₅₀ value determined in the presence of HSA [see Assay VII in the “Pharmacological methods” section for a description of a suitable assay procedure].

Compounds of the present invention modulate melanocortin receptors, and they are therefore believed to be particularly suited for the treatment of diseases or states which can be treated by a modulation of melanocortin receptor activity. In particular, compounds of the present invention are believed to be suited for the treatment of diseases or states via activation of MC4.

Another aspect or embodiment of the invention relates to:

104. A pharmaceutical composition comprising a compound according to any of embodiments 1-103.
105. A pharmaceutical composition according to embodiment 104, further comprising one or more additional therapeutically active compounds or substances.
106. A pharmaceutical composition according to embodiment 104, further comprising a GLP-1 compound.
107. A pharmaceutical composition according to embodiment 106, wherein the GLP-1 compound is selected from the group consisting of:
N-epsilon26-((S)-4-Carboxy-4-hexadecanoylamino-butyryl)[Arg34]GLP-1-(7-37):

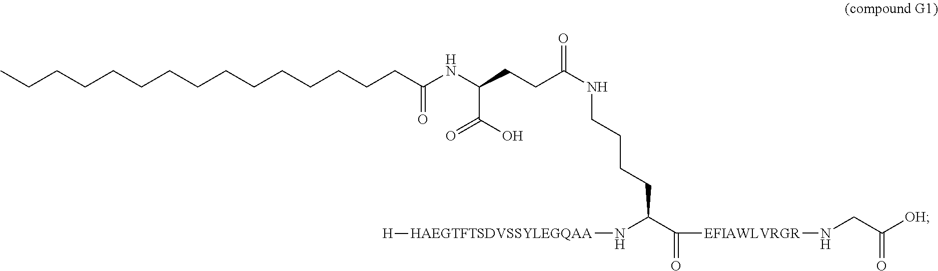

N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34,Lys37]GLP-1-(7-37):

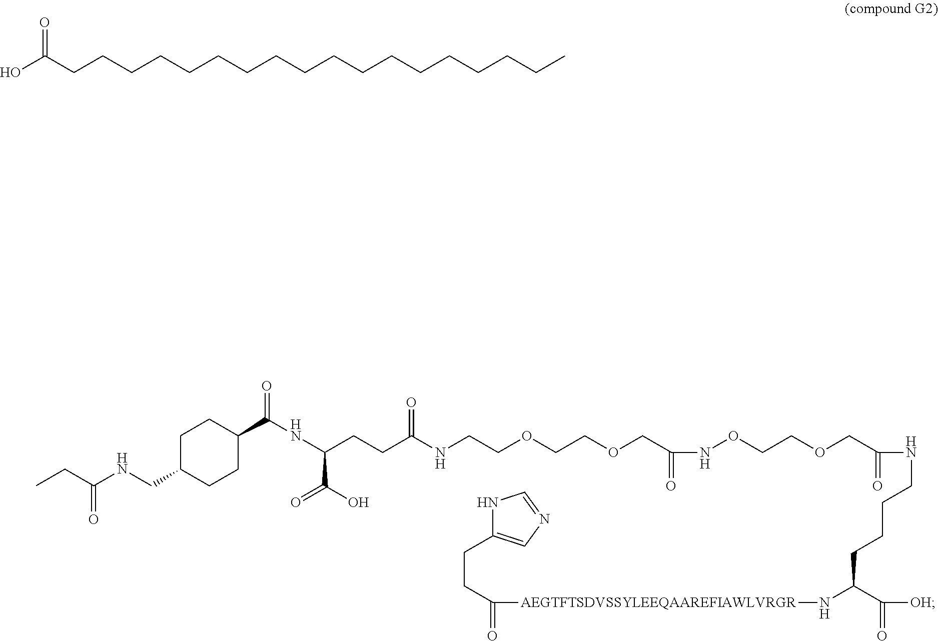

N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib8,Arg34]G canoylamino)butyrylamino]ethoxy}ethoxy)acetylamino]ethoxy}ethoxy)acetyl][Aib8,Arg34]GLP-1-(7-37):

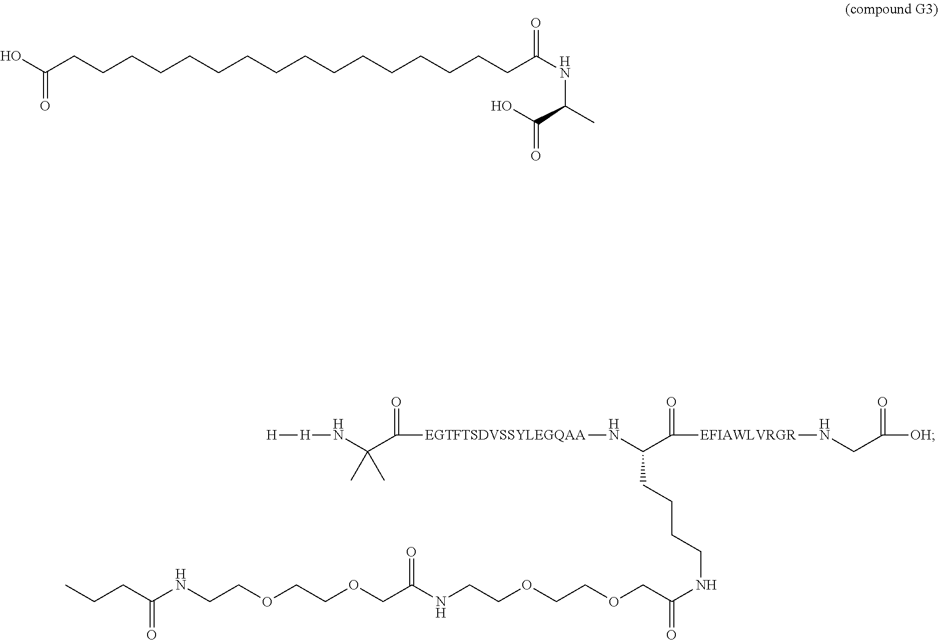

N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35,Lys37]GLP-1-(7-37):

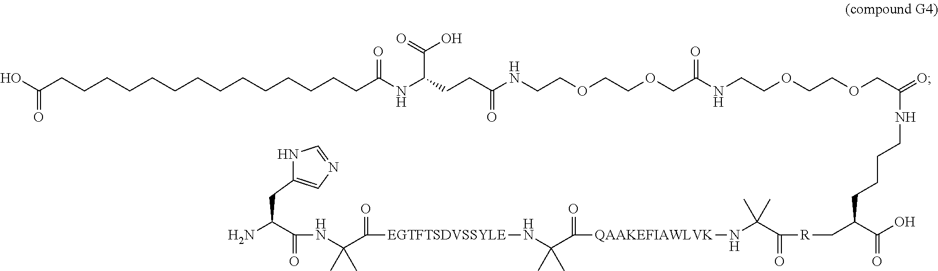

and their pharmaceutically acceptable salts, amides, alkyls, or esters.
108. A pharmaceutical composition according to embodiments 104-107, in unit dosage form comprising from about 0.05 mg to about 1000 mg, such as from about 0.1 mg to about 500 mg, e.g. from about 0.5 mg to about 200 mg, of a compound according to any of embodiments 1-103.
109. A pharmaceutical composition according to any of embodiments 104-108, wherein the pH is neutral to weakly basic.
110. A pharmaceutical composition according to any of embodiments 104-108, wherein the pH is from about 7.0 to about 8.0.
111. A pharmaceutical composition according to any of embodiments 104-108, wherein the pH is about 7.5.

Among further aspects or embodiments of the present invention are the following:

112. A compound according to any of embodiments 1-103, optionally in combination with one or more additional therapeutically active compounds, for use in delaying the progression from impaired glucose tolerance (IGT) to type 2 diabetes.
113. A compound according to any of embodiments 1-103, optionally in combination with one or more additional therapeutically active compounds, for use in delaying the progression from non-insulin-requiring type 2 diabetes to insulin-requiring type 2 diabetes.
114. A compound according to any of embodiments 1-103, optionally in combination with one or more additional therapeutically active compounds, for use in treating obesity or preventing overweight.
115. A compound according to any of embodiments 1-103, optionally in combination with one or more additional therapeutically active compounds, for use in regulating appetite.
116. A compound according to any of embodiments 1-103, optionally in combination with one or more additional therapeutically active compounds, for use in inducing satiety.
117. A compound according to any of embodiments 1-103, optionally in combination with one or more additional therapeutically active compounds, for preventing weight gain after successful weight loss.
118. A compound according to any of embodiments 1-103, optionally in combination with one or more additional therapeutically active compounds, for use in increasing energy expenditure.
119. A pharmaceutical composition according to any of embodiments 104-111, for use in delaying the progression from impaired glucose tolerance (IGT) to type 2 diabetes.
120. A pharmaceutical composition according to any of embodiments 104-111, for use in delaying the progression from non-insulin-requiring type 2 diabetes to insulin-requiring type 2 diabetes.
121. A pharmaceutical composition according to any of embodiments 104-111, for use in treating obesity or preventing overweight.
122. A pharmaceutical composition according to any of embodiments 104-111, for use in regulating appetite.
123. A pharmaceutical composition according to any of embodiments 104-111, for use in inducing satiety.
124. A pharmaceutical composition according to any of embodiments 104-111, for preventing weight gain after successful weight loss.
125. A pharmaceutical composition according to any of embodiments 104-111, for use in increasing energy expenditure.
126. A method of delaying the progression from IGT to type 2 diabetes, comprising administering to a patient in need thereof an effective amount of a compound according to any of embodiments 1-103, optionally in combination with one or more additional therapeutically active compounds.
127. A method of delaying the progression from non-insulin-requiring type 2 diabetes to insulin-requiring type 2 diabetes, comprising administering to a patient in need thereof an effective amount of a compound according to any of embodiments 1-103, optionally in combination with one or more additional therapeutically active compounds.
128. A method of treating obesity or preventing overweight, comprising administering to a patient in need thereof an effective amount of a compound according to any of embodiments 1-103, optionally in combination with one or more additional therapeutically active compounds.
129. A method of regulating appetite, comprising administering to a patient in need thereof an effective amount of a compound according to any of embodiments 1-103, optionally in combination with one or more additional therapeutically active compounds.
130. A method of inducing satiety, comprising administering to a patient in need thereof an effective amount of a compound according to any of embodiments 1-103, optionally in combination with one or more additional therapeutically active compounds.
131. A method of preventing weight gain after successfully having lost weight, comprising administering to a patient in need thereof an effective amount of a compound according to any of embodiments 1-103, optionally in combination with one or more additional therapeutically active compounds.
132. A method of increasing energy expenditure, comprising administering to a patient in need thereof an effective amount of a compound according to any of embodiments 1-103, optionally in combination with one or more additional therapeutically active compounds.
133. A method of delaying the progression from IGT to type 2 diabetes, comprising administering to a patient in need thereof an effective amount of a pharmaceutical composition according to any of embodiments 104-111.
134. A method of delaying the progression from non-insulin-requiring type 2 diabetes to insulin-requiring type 2 diabetes, comprising administering to a patient in need thereof an effective amount of a pharmaceutical composition according to any of embodiments 104-111.
135. A method of treating obesity or preventing overweight, comprising administering to a patient in need thereof an effective amount of a pharmaceutical composition according to any of embodiments 104-111.
136. A method of regulating appetite, comprising administering to a patient in need thereof an effective amount of a pharmaceutical composition according to any of embodiments 104-111.
137. A method of inducing satiety, comprising administering to a patient in need thereof an effective amount of a pharmaceutical composition according to any of embodiments 104-111.
138. A method of preventing weight gain after successfully having lost weight, comprising administering to a patient in need thereof an effective amount of a pharmaceutical composition according to any of embodiments 104-111.
139. A method of increasing energy expenditure, comprising administering to a patient in need thereof an effective amount of a pharmaceutical composition according to any of embodiments 104-111.

Among yet further aspects or embodiments of the present invention are the following:

140. A compound according to any of embodiments 1-103, optionally in combination with one or more additional therapeutically active compounds, for use in treating a disease or state related to overweight or obesity.
141. A compound according to any of embodiments 1-103, optionally in combination with one or more additional therapeutically active compounds, for use in treating bulimia.
142. A compound according to any of embodiments 1-103, optionally in combination with one or more additional therapeutically active compounds, for treating binge-eating.
143. A compound according to any of embodiments 1-103, optionally in combination with one or more additional therapeutically active compounds, for use in treating a disease or state selected from atherosclerosis, hypertension, type 2 diabetes, impaired glucose tolerance (IGT), dyslipidemia, coronary heart dis-ease, gallbladder disease, gall stone, osteoarthritis, cancer, sexual dysfunction, hypthalamic amenorrhea and risk of premature death.
144. A compound according to any of embodiments 1-103, optionally in combination with one or more additional therapeutically active compounds, for providing neuronal protection, for having an effect on ischemic heart disease, cerebral ischemia or anti-inflammatory effects and for the treatment of autoimmune diseases, e.g. multiple sclerosis.
145. A pharmaceutical composition according to any of embodiments 104-111, for use in treating a disease or state related to overweight or obesity.
146. A pharmaceutical composition according to any of embodiments 104-111, for use in treating bulimia.
147. A pharmaceutical composition according to any of embodiments 104-111, for use in treating binge-eating.
148. A pharmaceutical composition according to any of embodiments 104-111, for use in treating a disease or state selected from atherosclerosis, hypertension, type 2 diabetes, impaired glucose tolerance (IGT), dyslipidemia, coronary heart dis-ease, gallbladder disease, gall stone, osteoarthritis, cancer, sexual dysfunction, hypthalamic amenorrhea and risk of premature death.
149. A pharmaceutical composition according to any of embodiments 104-111, for use in providing neuronal protection, for having an effect on ischemic heart disease, cerebral ischemia or anti-inflammatory effects and for the treatment of autoimmune diseases, e.g. multiple sclerosis.
150. A method of treating a disease or state related to overweight or obesity, comprising administering to a patient in need thereof an effective amount of a compound according to any of embodiments 1-103, optionally in combination with one or more additional therapeutically active compounds.
151. A method of treating bulimia, comprising administering to a patient in need thereof an effective amount of a compound according to any of embodiments 1-103, optionally in combination with one or more additional therapeutically active compounds.
152. A method of treating binge-eating, comprising administering to a patient in need thereof an effective amount of a compound according to any of embodiments 1-103, optionally in combination with one or more additional therapeutically active compounds.
153. A method of treating a disease or state selected from atherosclerosis, hypertension, type 2 diabetes, impaired glucose tolerance (IGT), dyslipidemia, coronary heart disease, gallbladder disease, gall stone, osteoarthritis, cancer, sexual dysfunction, hypthalamic amenorrhea and risk of premature death, comprising administering to a patient in need thereof an effective amount of a compound according to any of embodiments 1-103, optionally in combination with one or more additional therapeutically active compounds.
154. A method for providing neuronal protection, for having an effect on ischemic heart disease, cerebral ischemia or anti-inflammatory effects and for the treatment of auto-immune diseases, e.g. multiple sclerosis., comprising administering to a patient in need thereof an effective amount of a compound according to any of embodiments 1-103, optionally in combination with one or more additional therapeutically active compounds.
155. A method of treating a disease or state related to overweight or obesity, comprising administering to a patient in need thereof an effective amount of a pharmaceutical composition according to any of embodiments 104-111.
156. A method of treating bulimia, comprising administering to a patient in need thereof an effective amount of a pharmaceutical composition according to any of embodiments 104-111.
157. A method of treating binge-eating, comprising administering to a patient in need thereof an effective amount of a pharmaceutical composition according to any of embodiments 104-111.
158. A method of treating a disease or state selected from atherosclerosis, hypertension, type 2 diabetes, impaired glucose tolerance (IGT), dyslipidemia, coronary heart disease, gallbladder disease, gall stone, osteoarthritis, cancer, sexual dysfunction, hypthalamic amenorrhea and risk of premature death, comprising administering to a patient in need thereof an effective amount of a pharmaceutical composition according to any of embodiments 104-111.
159. A method for providing neuronal protection, for having an effect on ischemic heart disease, cerebral ischemia or anti-inflammatory effects and for the treatment of auto-immune diseases, e.g. multiple sclerosis, comprising administering to a patient in need thereof an effective amount of a pharmaceutical composition according to any of embodiments 104-111.

Compounds of the present invention may be suited for the treatment of diseases in obese or overweight patients. Accordingly, a yet further aspect or embodiment of the invention relates to the following:

160. A compound according to any of embodiments 1-103, optionally in combination with one or more additional therapeutically active compounds, for use in treating, in an obese patient, a disease or state selected from atherosclerosis, hypertension, type 2 diabetes, IGT, dyslipidemia, coronary heart disease, gallbladder disease, gall stone, osteoarthritis, cancer, sexual dysfunction, hypthalamic amenorrhea, risk of premature death, neuronal protection, effect in ischemic heart disease or anti-inflammatory effects.
161. A pharmaceutical composition according to any of embodiments 104-111, for use in treating, in an obese patient, a disease or state selected from atherosclerosis, hypertension, type 2 diabetes, IGT, dyslipidemia, coronary heart disease, gallbladder disease, gall stone, osteoarthritis, cancer, sexual dysfunction, hypthalamic amenorrhea, risk of premature death, neuronal protection, effect in ischemic heart disease or anti-inflammatory effects.
162. A method of treating, in an obese patient, a disease or state selected from atherosclerosis, hypertension, type 2 diabetes, IGT, dyslipidemia, coronary heart disease, gallbladder disease, gall stone, osteoarthritis, cancer, sexual dysfunction, hypthalamic amenorrhea, risk of premature death, neuronal protection, effect in ischemic heart disease or anti-inflammatory effects, comprising administering to an obese patient in need thereof an effective amount of a compound according to any of embodiments 1-103, optionally in combination with one or more additional therapeutically active compounds.
163. A method of treating, in an obese patient, a disease or state selected from atherosclerosis, hypertension, type 2 diabetes, IGT, dyslipidemia, coronary heart disease, gallbladder disease, gall stone, osteoarthritis, cancer, sexual dysfunction, hypthalamic amenorrhea, risk of premature death, neuronal protection, effect in ischemic heart disease or anti-inflammatory effects, comprising administering to an obese patient in need thereof an effective amount of a pharmaceutical composition according to any of embodiments 104-111.

Yet further aspects or embodiments of the invention relate to:

164. A compound according to any of embodiments 112-118, 140-144 and 160, wherein said additional therapeutically active compound is selected from antidiabetic agents, antihyperlipidemic agents, antiobesity agents, antihypertensive agents and agents for the treatment of complications resulting from, or associated with, diabetes.
165. A pharmaceutical composition according to any of embodiments 105-111, 119-125, 145-149 and 161, wherein said additional therapeutically active compound is selected from antidiabetic agents, antihyperlipidemic agents, antiobesity agents, antihypertensive agents and agents for the treatment of complications resulting from, or associated with, diabetes.
166. A method according to any of embodiments 126-132, 150-154 and 162, wherein said additional therapeutically active compound is selected from antidiabetic agents, antihyperlipidemic agents, antiobesity agents, antihypertensive agents and agents for the treatment of complications resulting from, or associated with, diabetes.
167. A compound according to any of embodiments 1-103, 112-118, 140-144, 160 and 164, which is in a unit dosage form comprising from about 0.05 mg to about 1000 mg of said compound.
168. A pharmaceutical composition according to any of embodiments 104-111, 119-125, 145-149, 161 and 165, wherein said compound according to any of embodiments 1-103 is in a unit dosage form comprising from about 0.05 mg to about 1000 mg of said compound.
169. A method according to any of embodiments 126-132, 150-154, 162 and 166, wherein said compound according to any of embodiments 1-103 is administered to said patient in a unit dosage form comprising from about 0.05 mg to about 1000 mg of said compound.
170. A method according to any of embodiments 126-132, 150-154, 162, 166 and 169, wherein said compound according to any of embodiments 1-100 is administered to said patient, once daily.
171. A method according to any of embodiments 126-132, 150-154, 162, 166 and 169-170, wherein said compound according to any of embodiments 1-100 is administered to said patient once weekly.
172. A method of activating MC4 in a subject, the method comprising administering to said subject an effective amount of a compound according to any of embodiments 1-103.
173. A method according to any of embodiments 126-132, 150-154, 162, 166 and 169-171, wherein said compound according to any of embodiments 1-103 is administered parenterally, orally, nasally, buccally or sublingually.
174. A method according to any of embodiments 126-132, 150-154, 162, 166 and 169-171, wherein said compound according to any of embodiments 1-103 is administered parenterally or sublingually.

Yet another aspect or embodiment of the invention relates to the following:

175. A compound according to any of embodiments 1-103 for use in therapy.
176. A pharmaceutical composition according to any of embodiments 104-111 for use in therapy.
177. Use of a compound according to any of embodiments 1-103 in the manufacture of a medicament for delaying the progression from impaired glucose tolerance (IGT) to type 2 diabetes; delaying the progression from type 2 diabetes to insulin-requiring diabetes; treating obesity or preventing overweight; regulating appetite; inducing satiety; preventing weight regain after successful weight loss; increasing energy expenditure; treating a disease or state related to overweight or obesity; treating bulimia; treating binge-eating; treating atherosclerosis, hypertension, type 2 diabetes, IGT, dyslipidemia, coronary heart disease, gallbladder disease, gall stone, osteoarthritis, cancer, sexual dysfunction, hypthalamic amenorrhea or risk of premature death; or treating, in an obese patient, a disease or state selected from type 2 diabetes, IGT, dyspilidemia, coronary heart disease, gallbladder disease, gall stone, osteoarthritis, cancer, sexual dysfunction, risk of premature death; for providing neuronal protection, for having an effect on ischemic heart disease, cerebral ischemia or anti-inflammatory effects and for the treatment of autoimmune diseases, e.g. multiple sclerosis.

Compounds of the invention that act as MC4 agonists could have a positive effect on insulin sensitivity, on drug abuse (by modulating the reward system) and on hemorrhagic shock. Furthermore, MC3 and MC4 agonists have antipyretic effects, and both have been suggested to be involved in peripheral nerve regeneration. MC4 agonists are also known to reduce stress response. In addition to treating drug abuse, treating or preventing hemorrhagic shock, and reducing stress response, compounds of the invention may also be of value in treating alcohol abuse, treating stroke, treating ischemia and protecting against neuronal damage.

As already indicated, in all of the therapeutic methods or indications disclosed above, the compound of the present invention may be administered alone. However, it may also be administered in combination with one or more additional therapeutically active agents, substances or compounds, either sequentially or concomitantly.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from about 0.05 to about 1000 mg, preferably from about 0.1 to about 500 mg, such as from about 0.5 to about 200 mg of a compound of the invention.

Compounds of the invention comprise compounds that are believed to be well-suited to administration with longer intervals than, for example, once daily, thus, appropriately formulated compounds of the invention may be suitable for, e.g., twice-weekly or once-weekly administration by a suitable route of administration, such as one of the routes disclosed herein.

As described above, compounds of the present invention may be administered or applied in combination with one or more additional therapeutically active compounds or substances, and suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include insulin, insulin derivatives or analogues, GLP-1 (glucagon like peptide-1) derivatives or analogues [such as those disclosed in WO 98/08871 (Novo Nordisk A/S), which is incorporated herein by reference, or other GLP-1 analogues such as exenatide (Byetta, Eli Lilly/Amylin; AVE0010, Sanofi-Aventis), taspoglutide (Roche), albiglutide (Syncria, GlaxoSmithKline), amylin, amylin analogues (e.g. Symlin™/Pramlintide) as well as orally active hypoglycemic agents.

Suitable orally active hypoglycemic agents include: metformin, imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S) which are incorporated herein by reference; potassium channel openers such as ormitiglinide; potassium channel blockers such as nateglinide or BTS-67582; glucagon receptor antagonists such as those disclosed in WO 99/01423 and WO 00/39088 (Novo Nordisk A/S and Agouron Pharmaceuticals, Inc.), all of which are incorporated herein by reference; GLP-1 receptor agonists such as those disclosed in WO 00/42026 (Novo Nordisk A/S and Agouron Pharmaceuticals, Inc.), which are incorporated herein by reference; amylin analogues (agonists on the amylin receptor); DPP-IV (dipeptidyl peptidase-IV) inhibitors; PTPase (protein tyrosine phosphatase) inhibitors; glucokinase activators, such as those described in WO 02/08209 to Hoffmann La Roche; inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include: α-glucosidase inhibitors, e.g. voglibose, emiglitate, miglitol or acarbose; glycogen phosphorylase inhibitors, e.g. the compounds described in WO 97/09040 (Novo Nordisk A/S); glucokinase activators; agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y receptor 1 and/or 5) antagonists, MC3 (melanocortin receptor 3) agonists, MC3 antagonists, MC4 (melanocortin receptor 4) agonists, orexin receptor antagonists, TNF (tumor necrosis factor) agonists, CRF (corticotropin releasing factor) agonists, CRF BP (corticotropin releasing factor binding protein) antagonists, urocortin agonists, neuromedin U analogues (agonists on the neuromedin U receptor subtypes 1 and 2), β3 adrenergic agonists such as CL-316243, AJ-9677, GW-0604, LY362884, LY377267 or AZ-40140, MC1 (melanocortin receptor 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, 5HT6 agonists, 5HT2c agonists such as APD356 (U.S. Pat. No. 6,953,787), bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4 analogues, GLP-1 analogues, ciliary neurotrophic factor, amylin analogues, peptide YY₃₋₃₆ (PYY3-36) (Batterham et al, Nature 418, 650-654 (2002)), PYY3-36 analogues, NPY Y2 receptor agonists, NPY Y4 receptor agonists and substances acting as combined NPY Y2 and NPY Y4 agonists, FGF21 and analogues thereof, μ-opioid receptor antagonists, oxyntomodulin or analogues thereof.

Further suitable antiobesity agents are bupropion (antidepressant), topiramate (anti-convulsant), ecopipam (dopamine D1/D5 antagonist) and naltrexone (opioid antagonist), and combinations thereof. Combinations of these antiobesity agents would be e.g.: phentermine+topiramate, bupropion sustained release (SR)+naltrexone SR, zonisamide SR and bupropion SR. Among embodiments of suitable antiobesity agents for use in a method of the invention as additional therapeutically active substances in combination with a compound of the invention are leptin and analogues or derivatives of leptin.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In one embodiment, the present invention relates to the combination of a compound of the present invention and a glucagon-like peptide 1 (GLP-1) compound.

In one embodiment, the present invention relates to the combination of a compound of the present invention and exendin-4.

In one embodiment, the present invention relates to a compound of the present invention and a GLP-1 compound in combination, for the preparation of a medicament for the treatment of diabetes and/or obesity.

In one embodiment, the present invention relates to a compound of the present invention and a exendin-4 compound in combination, for the preparation of a medicament for the treatment of diabetes and/or obesity.

In another embodiment the invention relates to pharmaceutical compositions comprising a compound of the present invention and GLP-1 compounds in combination, together with a pharmaceutically acceptable carrier.

In another embodiment the invention relates to pharmaceutical compositions comprising a compound of the present invention and exendin-4 compounds in combination, together with a pharmaceutically acceptable carrier.

GLP-1 is an incretin hormone produced by the endocrine cells of the intestine following ingestion of food. GLP-1 is a regulator of glucose metabolism, and the secretion of insulin from the beta cells of the islets of Langerhans in the pancreas. GLP-1 also causes insulin secretion in the diabetic state. The half-life in vivo of GLP-1 itself is, however, very short, thus, ways of prolonging the half-life of GLP-1 in vivo has attracted much attention.

WO 98/08871 discloses protracted GLP-1 analogues and derivatives based on human GLP-1(7-37) (amino acids 1-31 of SEQ ID NO:3) which have an extended half-life, including liraglutide, a GLP-1 derivative for once daily administration developed by Novo Nordisk A/S and expected to be marketed soon for the treatment of type 2 diabetes.

Exenatide is a commercial incretin mimetic for the treatment of diabetes mellitus type 2 which is manufactured and marketed by Amylin Pharmaceuticals and Eli Lilly & Co. Exenatide is based on exendin-4(7-45) (amino acids 1-39 of SEQ ID NO:4), a hormone found in the saliva of the Gila monster. It displays biological properties similar to human GLP-1. U.S. Pat. No. 5,424,286 relates i.a. to a method of stimulating insulin release in a mammal by administration of exendin-4(7-45) (SEQ ID NO:1 in the U.S. patent).

The term “GLP-1 compound” as used herein refers to human GLP-1(7-37) (amino acids 1-31 of SEQ ID NO:3), exendin-4(7-45) (amino acids 1-39 of SEQ ID NO:4), as well as analogues, fusion peptides, and derivatives thereof, which maintain GLP-1 activity.

As regards position numbering in GLP-1 compounds: for the present purposes any amino acid substitution, deletion, and/or addition is indicated relative to the sequences of SEQ ID NO:3, and/or 4. However, the numbering of the amino acid residues in the sequence listing always starts with no. 1, whereas for the present purpose we want, following the established practice in the art, to start with amino acid residue no. 7 and assign number 7 to it. Therefore, generally, any reference herein to a position number of the GLP-1(7-37) or exendin-4 sequence is to the sequence starting with His at position 7 in both cases, and ending with Gly at position 37, or Ser at position 45, respectively.

GLP-1 compounds may be prepared as exemplified in example 71.

GLP-1 activity may be determined using any method known in the art, e.g. the assay (XI) herein (stimulation of cAMP formation in a cell line expressing the human GLP-1 receptor).

Furthermore, the GLP-1 compound is a compound which may:

i) comprise at least one of the following: DesaminoHis7, Aib8, Aib22, Arg26, Aib35, and/or Lys37;

ii) be a GLP-1 derivative comprising an albumin binding moiety which comprises at least one, preferably at least two, more preferably two, free carboxylic acid groups; or a pharmaceutically acceptable salt thereof;

iii) be a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) be selected from the group consisting of compounds N-epsilon26-((S)-4-Carboxy-4-hexadecanoylamino-butyryl)[Arg34]GLP-1-(7-37):

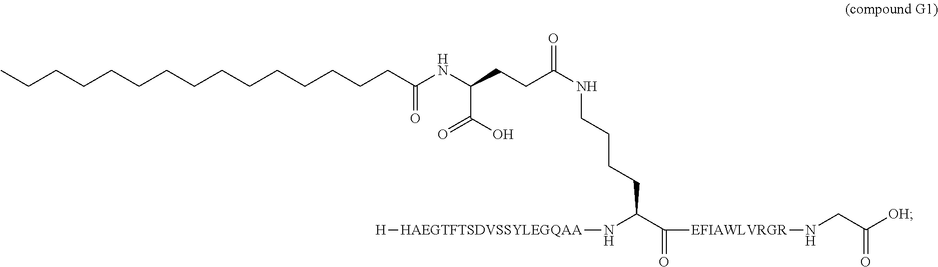

N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamin o]ethoxy}ethoxy)acetyl][DesaminoHis7,Glu22,Arg26,Arg34,Lys37]GLP-1-(7-37):

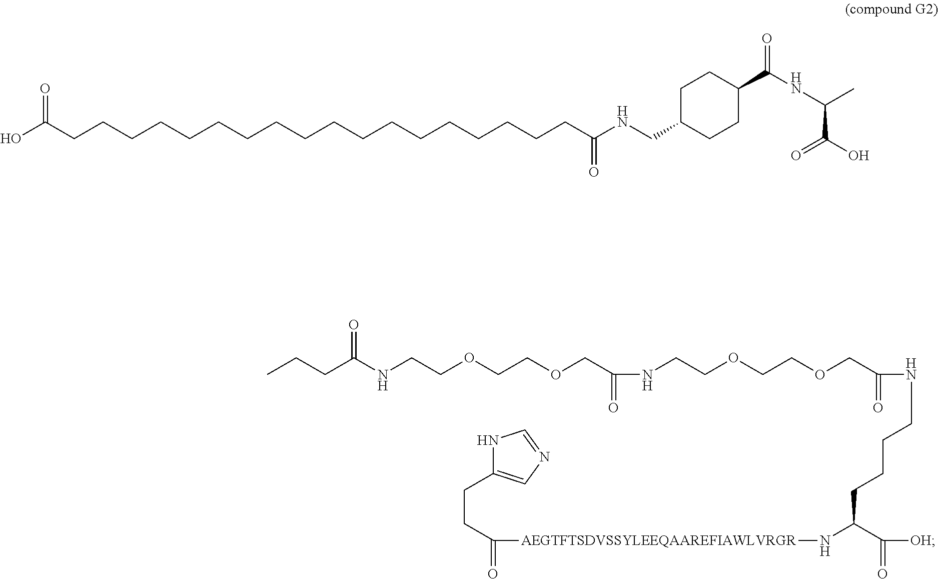

N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib8,Arg34]G LP-1-(7-37):

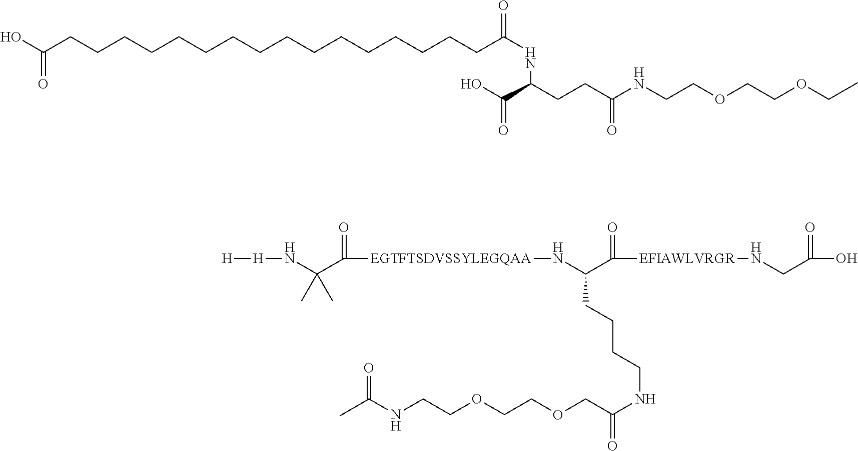

N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35, Lys37]GLP-1-(7-37):

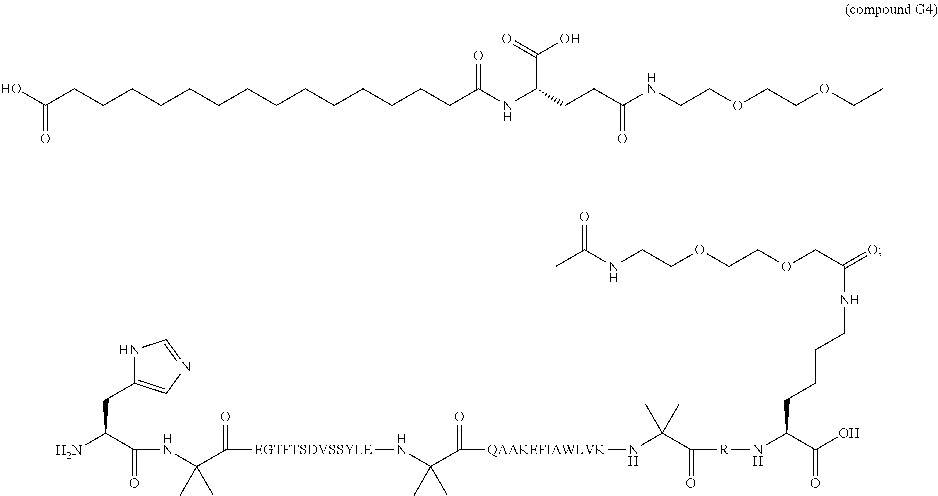

and their pharmaceutically acceptable salts, amides, alkyls, or esters.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In the case, in particular, of administration of a compound of the invention, optionally in combination with one or more additional therapeutically active compounds or substances as disclosed above, for a purpose related to treatment or prevention of obesity or overweight, i.e. related to reduction or prevention of excess adiposity, it may be of relevance to employ such administration in combination with surgical intervention for the purpose of achieving weight loss or preventing weight gain, e.g. in combination with bariatric surgical intervention. Examples of frequently used bariatric surgical techniques include, but are not limited to, the following: vertical banded gastroplasty (also known as “stomach stapling”), wherein a part of the stomach is stapled to create a smaller pre-stomach pouch which serves as a new stomach; gastric banding, e.g. using an adjustable gastric band system (such as the Swedish Adjustable Gastric Band (SAGB), the LAP-BAND™ or the MIDband™), wherein a small prestomach pouch which is to serve as a new stomach is created using an elastomeric (e.g. silicone) band which can be adjusted in size by the patient; and gastric bypass surgery, e.g. “Roux-en-Y” bypass wherein a small stomach pouch is created using a stapler device and is connected to the distal small intestine, the upper part of the small intestine being reattached in a Y-shaped configuration.

Another technique which is within the scope of the term “bariatric surgery” and variants thereof (e.g. “weight-loss surgery”, “weight-loss surgical intervention” “weight-loss surgical procedure”, “bariatric surgical intervention”, “bariatric surgical procedure” and the like) as employed in the context of the present invention is gastric balloon surgery, wherein an inflatable device resembling a balloon is introduced into the stomach and then inflated, the purpose being to reduce the accessible volume within the stomach to create a sensation of satiety in the patient at an earlier stage than normal during food intake, and thereby cause a reduction in food intake by the patient.

Another technique which is within the scope of the term “bariatric surgery” is EndoBarrier™ technology, which is a proprietary platform of products for the treatment of type 2 diabetes and obesity. The EndoBarrier Gastrointestinal Liner works by creating a physical barrier between ingested food and the intestinal wall, which may change how hormonal signals that originate in the intestine are activated, mimicking the effects of gastric bypass procedure. Weight loss is enhanced by combining the EndoBarrier Gastrointestinal Liner with the customizable EndoBarrier Flow Restrictor. Combination of the EndoBarrier Gastrointestinal Liner with the EndoBarrier Flow Restrictor was shown to affect multiple mechanisms of action.

All of the above-mentioned techniques are in principle reversible. Non-limiting examples of additional, irreversible and consequently generally less frequently employed techniques of relevance in the present context include biliopancreatic diversion and sleeve gastrectomy (the latter of which may also be employed in conjunction with duodenal switch), both of which entail surgical resection of a substantial portion of the stomach.

The administration of a compound of the invention (optionally in combination with one or more additional therapeutically active compounds or substances as disclosed above) may take place for a period prior to carrying out the bariatric surgical intervention in question and/or for a period of time subsequent thereto. In many cases it may be preferable to begin administration of a compound of the invention after bariatric surgical intervention has taken place.

The treatment of obesity might be possible by using long-acting melanocortin 4 receptor agonists (MC4 agonists) comprising a peptide part and a fatty acid or alkyltetrazole chain as described in e.g. WO2007/009894, WO2008/087186 and WO2008/087187. These compounds have more basic than acidic residues, resulting in good solubility at acidic pH, but poor solubility at neutral or weakly basic pH. Solubility at pH from 6 to 9 is considered to be an advantage, since this could improve local tolerance and make it possible to combine the MC4 agonist with other drugs, soluble only at neutral to weakly basic pH.

The compounds of the present invention have a novel chemical structure. The compounds of the present invention comprise a macrocyclic peptide part, a linear peptide part linked to the C-terminus of the macrocycle and a fatty acid derivative in the C-terminal region of the peptide. The fatty acid derivative can be an alkyltetrazole. The peptide backbone has some similarity with the hormone β-MSH.

The primary function of the macrocyclic peptide part is to bind to the MC4 receptor, resulting in activation of the MC4 receptor, with selectivity for MC4 over MC1, MC3 and MC5 receptors. One of the functions of the covalently linked fatty acid part is to bind reversibly to human serum albumin (HSA), resulting in long in vivo half-life of the compound. The fatty acid part, however, is hydrophobic and therefore disadvantageous in terms of solubility. To ensure sufficient solubility of the compound, the linear peptide part with its overall negative charge is needed. The linear peptide part is also important for receptor binding and selectivity and is therefore limited to certain amino acid sequences. The fatty acid part may contribute to receptor binding and selectivity as well. The desired balance between MC4 potency, receptor selectivity, water-solubility and long lasting in vivo effect is achieved by the covalent combination of macrocyclic peptide part, linear peptide part linked to the C-terminus of the macrocycle and fatty acid part, according to the above-described embodiments of compounds of the invention.

The compounds of the present invention are negatively charged at pH 7.5, based on the number of acidic and basic residues present in the molecule. Therefore, all compounds of the present invention are expected to be soluble at pH 7.5 and above.

The compounds of the present invention have high MC4 receptor potency and higher MC4 receptor selectivity in relation to previously disclosed peptides in the art. The peptides of the present invention also have prolonged in vivo half-life. The compounds of the present invention can be a soluble MC4 receptor agonist, for example with solubility of at least 0.2 mmol/l, at least 0.5 mmol/l, at least 2 mmol/l, at least 4 mmol/l, at least 8 mmol/l, at least 10 mmol/l, or at least 15 mmol/l, at pH 7.5.

The compounds of the present invention can be a soluble MC4 receptor agonist, for example with solubility of at least 0.2 mmol/l, at least 0.5 mmol/l, at least 2 mmol/l, at least 4 mmol/l, at least 8 mmol/l, at least 10 mmol/l, or at least 15 mmol/l at pH 7.5.

In the present context, if not stated otherwise, the terms “soluble”, “solubility”, “soluble in aquous solution”, “aqueous solubility”, “water soluble”, “water-soluble”, “water solubility” and “water-solubility”, refer to the solubility of a compound in water or in an aqueous salt or aqueous buffer solution, for example a 10 mM phosphate solution, or in an aqueous solution containing other compounds, but no organic solvents.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) above 25 are to be regarded as obese.

The use of a prefix of the type “C_{x-y}” preceding the name of a radical, such as in C_{x-y}alkyl (e.g. C₆₋₂₀alkyl) is intended to indicate a radical of the designated type having from x to y carbon atoms.

The term “alkyl” as used herein refers to a straight-chain, branched and/or cyclic, saturated monovalent hydrocarbon radical.

The term “alkenyl” as used herein refers to a straight-chain, branched and/or cyclic, monovalent hydrocarbon radical comprising at least one carbon-carbon double bond.

The term “alkynyl” as used herein refers to a straight-chain, branched and/or cyclic, monovalent hydrocarbon radical comprising at least one carbon-carbon triple bond, and it may optionally also comprise one or more carbon-carbon double bonds.

The term “alkylene” as used herein refers to a straight-chain, branched and/or cyclic, saturated bivalent hydrocarbon radical.

The term “alkenylene” as used herein refers to a straight-chain, branched and/or cyclic, bivalent hydrocarbon radical comprising at least one carbon-carbon double bond.

The term “alkynylene” as used herein refers to a straight-chain, branched and/or cyclic, bivalent hydrocarbon radical comprising at least one carbon-carbon triple bond, and it may optionally also comprise one or more carbon-carbon double bonds.

The term “alkoxy” as used herein is intended to indicate a radical of the formula —OR′, wherein R′ is alkyl as indicated above.

In the present context, the term “aryl” is intended to indicate a carbocyclic aromatic ring radical or a fused aromatic ring system radical wherein at least one of the rings is aromatic. Typical aryl groups include phenyl, biphenylyl, naphthyl, and the like.

The term “halogen” is intended to indicate members of the 7^{th} main group of the periodic table of the elements, which includes fluorine, chlorine, bromine and iodine (corresponding to fluoro, chloro, bromo and iodo substituents, respectively).

The term “tetrazol-5-yl” is intended to indicate 1H-tetrazol-5-yl or 2H-tetrazol-5-yl.

In the present context, common rules for peptide nomenclature based on the three letter amino acid code apply, unless exceptions are specifically indicated. Briefly, the central portion of the amino acid structure is represented by the three letter code (e.g. Ala, Lys) and L-configuration is assumed, unless D-configuration is specifically indicated by “D-” followed by the three letter code (e.g. D-Ala, D-Lys). A substituent at the amino group replaces one hydrogen atom and its name is placed before the three letter code, whereas a C-terminal substituent replaces the carboxylic hydroxy group and its name appears after the three letter code. For example, “acetyl-Gly-Gly-NH₂” represents CH₃—C(═O)—NH—CH₂—C(═O)—NH—CH₂—C(═O)—NH₂. Unless indicated otherwise, amino acids with additional amino or carboxy groups in the side chains (such as Lys, Orn, Dap, Glu, Asp and others) are connected to their neighboring groups by amide bonds formed at the N-2 (α-nitrogen) atom and the C-1 (C═O) carbon atom.

When two amino acids are said to be bridged, it is intended to indicate that functional groups in the side chains of the two respective amino acids have reacted to form a covalent bond.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The amino acid abbreviations used in the present context have the following meanings:

| | |
|---|---|
| Ala | Alanine |
| β-Ala | 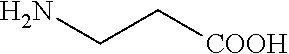 |
| Asn | Asparagine |
| Asp | Aspartic acid |
| β-Asp | 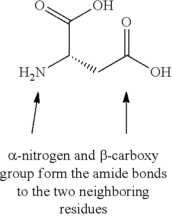 |
| Arg | Arginine |
| Cit | Citrulline |
| Cys | Cysteine |
| Dab | (S)-2,4-diaminobutyric acid |
| Dab (biscar- boxy- methyl) | 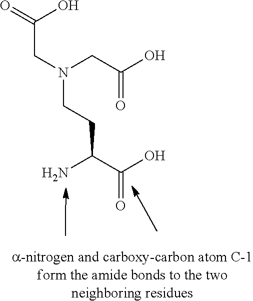 [SMILES: N[C@@H](CCN(CC(=O)O)CC(=O)O)C(=O)O] |
| Dab (BCMA) | 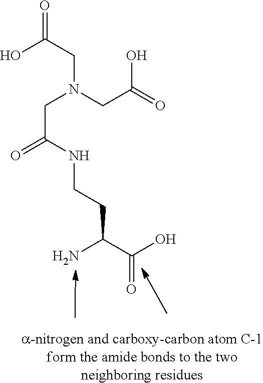 [SMILES: N[C@@H](CCNC(=O)CN(CC(=O)O)CC(=O)O)C(=O)O] |
| Dab(iPr) | 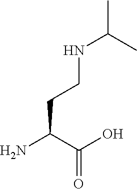 |
| Dab(Me₂) | 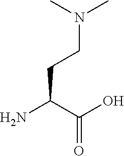 |
| Dap | (S)-2,3-diaminopropionic acid |
| Dap (biscar- boxy- methyl) | 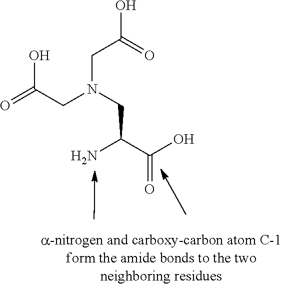 [SMILES: N[C@@H](CN(CC(=O)O)CC(=O)O)C(=O)O] |
| Dap (BCMA) | 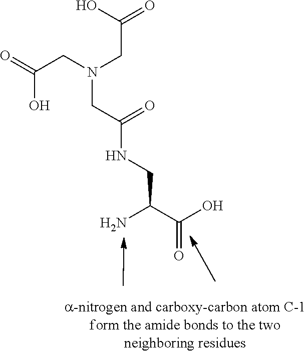 [SMILES: N[C@@H](CNC(=O)CN(CC(=O)O)CC(=O)O)C(=O)O] |
| Dap(iPr) | 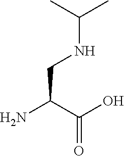 |
| Dap(Me₂) | 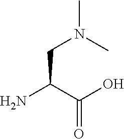 |
| D-β-Asp | 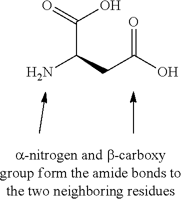 |
| D-Dap | (R)-2,3-diaminopropionic acid |
| D-γ-Glu | 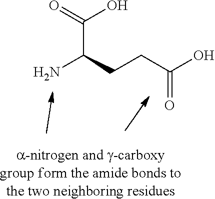 |
| D-Phe | 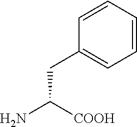 [SMILES: N[C@H](Cc1ccccc1)C(=O)O] |
| Gln | Glutamine |
| Glu | Glutamic acid |
| γ-Glu | 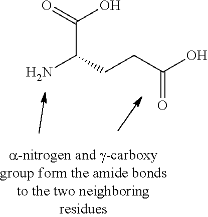 |
| Gly | Glycine |
| His | Histidine |
| homoArg | 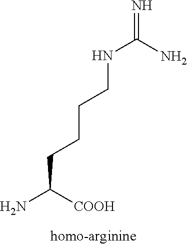 [SMILES: N=C(N)NCCCC[C@H](N)C(=O)O] |
| homoCys | 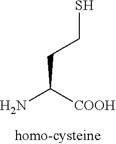 |
| homoLys | 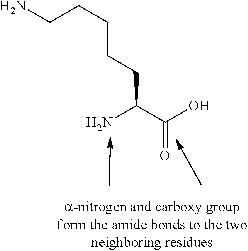 [SMILES: NCCCCC[C@H](N)C(=O)O] |
| homoLys (biscar- boxy- methyl) | 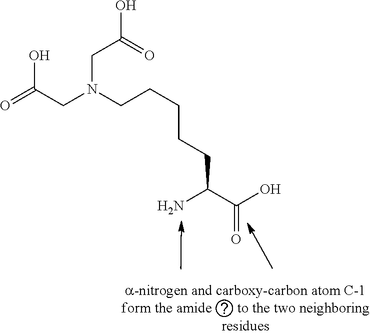 [SMILES: N[C@@H](CCCCCN(CC(=O)O)CC(=O)O)C(=O)O] |
| homoLys (BCMA) | 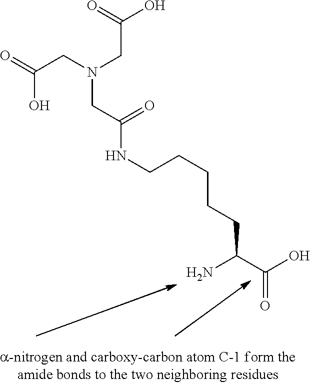 [SMILES: N[C@@H](CCCCCNC(=O)CN(CC(=O)O)CC(=O)O)C(=O)O] |
| homoSer | 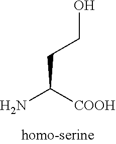 |
| Hyp | 4-hydroxyproline |
| Ile | Isoleucine |
| Leu | Leucine |
| Lys | Lysine |
| Lys (biscar- boxy- methyl) | 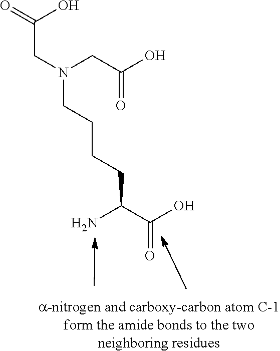 [SMILES: N[C@@H](CCCCN(CC(=O)O)CC(=O)O)C(=O)O] |
| Lys (BCMA) | 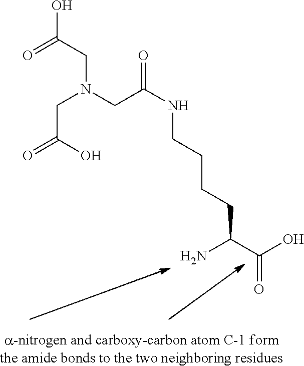 [SMILES: N[C@@H](CCCCNC(=O)CN(CC(=O)O)CC(=O)O)C(=O)O] |
| Met | Methionine |
| 2-Nal | 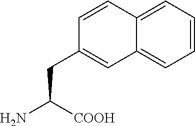 [SMILES: N[C@@H](Cc1ccc2ccccc2c1)C(=O)O] |
| Nle | 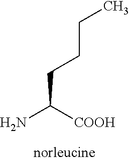 |
| Orn | Ornithine |
| Orn (biscar- boxy- methyl) | 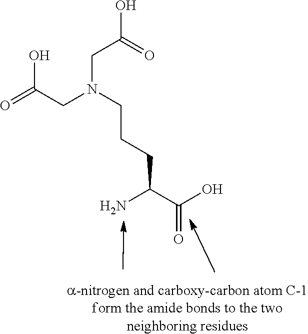 [SMILES: N[C@@H](CCCN(CC(=O)O)CC(=O)O)C(=O)O] |
| Orn (BCMA) | 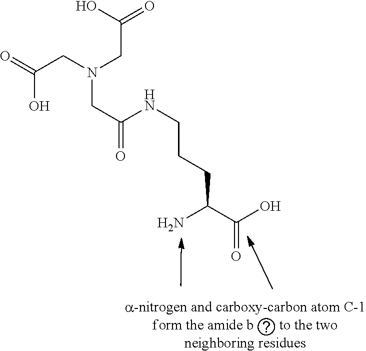 [SMILES: N[C@@H](CCCNC(=O)CN(CC(=O)O)CC(=O)O)C(=O)O] |
| Phe | Phenylalanine |
| Pro | Proline |
| Ser | Serine |
| Thr | Threonine |
| Tyr | Tyrosine |
| Trp | Tryptophan |
| Val | Valine |

BCMA = [bis(carboxymethyl)amino]acetyl
Amino acid abbreviations beginning with D- followed by a three letter code, such as D-Ser, D-His and so on, refer to the D-enantiomer of the corresponding amino acid, for example D-serine, D-histidine and so on.

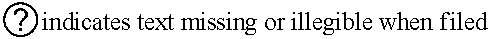

### Pharmaceutical Compositions

As already mentioned, one aspect of the present invention provides a pharmaceutical composition (formulation) comprising a compound of the present invention. Appropriate embodiments of such formulations will often contain a compound of the invention in a concentration of from 10⁻³ mg/ml to 200 mg/ml, such as, e.g., from 10⁻¹ mg/ml to 100 mg/ml. The pH in such a formulation of the invention will typically be in the range of 2.0 to 10.0. The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

In another embodiment, a pharmaceutical composition (formulation) of the invention may be a freeze-dried (i.e. lyophilized) formulation intended for reconstitution by the physician or the patient via addition of solvents and/or diluents prior to use.

In a further embodiment, a pharmaceutical composition (formulation) of the invention may be a dried formulation (e.g. freeze-dried or spray-dried) ready for use without any prior dissolution.

In a further aspect, the invention relates to a pharmaceutical composition (formulation) comprising an aqueous solution of a compound of the present invention, and a buffer, wherein the compound of the invention is present in a concentration of 0.1-100 mg/ml or above, and wherein the formulation has a pH from about 2.0 to about 10.0.

In another embodiment of the invention, the pH of the formulation has a value selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In a further embodiment, the buffer in a buffered pharmaceutical composition of the invention may comprise one or more buffer substances selected from the group consisting of sodium acetate, sodium carbonate, citrates, glycylglycine, histidine, glycine, lysine, arginine, sodium dihydrogen phosphate, disodium hydrogen phosphate, sodium phosphate, tris(hydroxymethyl)aminomethane (TRIS), bicine, tricine, malic acid, succinates, maleic acid, fumaric acid, tartaric acid and aspartic acid. Each one of these specific buffers constitutes an alternative embodiment of the invention.

In another embodiment, a pharmaceutical composition of the invention may comprise a pharmaceutically acceptable preservative, e.g. one or more preservatives selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thiomerosal, bronopol, benzoic acid, imidurea, chlorohexidine, sodium dehydroacetate, chlorocresol, ethyl p-hydroxybenzoate, benzethonium chloride and chlorphenesine (3p-chlorphenoxypropane-1,2-diol). Each one of these specific preservatives constitutes an alternative embodiment of the invention. In a further embodiment of the invention the preservative is present in a concentration from 0.1 mg/ml to 20 mg/ml. In still further embodiments of such a pharmaceutical composition of the invention, the preservative is present in a concentration in the range of 0.1 mg/ml to 5 mg/ml, a concentration in the range of 5 mg/ml to 10 mg/ml, or a concentration in the range of 10 mg/ml to 20 mg/ml. The use of a preservative in pharmaceutical compositions is well known to the skilled person. For convenience, reference is made in this respect to Remington: The Science and Practice of Pharmacy, 20^{th} edition, 2000.

In a further embodiment of the invention the formulation further comprises a tonicity-adjusting agent, i.e. a substance added for the purpose of adjusting the tonicity (osmotic pressure) of a liquid formulation (notably an aqueous formulation) or a reconstituted freeze-dried formulation of the invention to a desired level, normally such that the resulting, final liquid formulation is isotonic or substantially isotonic. Suitable tonicity-adjusting agents may be selected from the group consisting of salts (e.g. sodium chloride), sugars and sugar alcohols (e.g. mannitol), amino acids (e.g. glycine, histidine, arginine, lysine, isoleucine, aspartic acid, tryptophan or threonine), alditols [e.g. glycerol (glycerine), 1,2-propanediol (propyleneglycol), 1,3-propanediol or 1,3-butanediol], polyethyleneglycols (e.g. PEG 400) and mixtures thereof.

Any sugar, such as a mono-, di- or polysaccharide, or a water-soluble glucan, including for example fructose, glucose, mannose, sorbose, xylose, maltose, lactose, sucrose, trehalose, dextran, pullulan, dextrin, cyclodextrin, soluble starch, hydroxyethyl starch or carboxymethylcellulose-sodium, may be used; in one embodiment, sucrose may be employed. Sugar alcohols (polyols derived from mono-, di-, oligo- or polysaccharides) include, for example, mannitol, sorbitol, inositol, galactitol, dulcitol, xylitol, and arabitol. In one embodiment, the sugar alcohol employed is mannitol. Sugars or sugar alcohols mentioned above may be used individually or in combination. There is no fixed limit to the amount used, as long as the sugar or sugar alcohol is soluble in the liquid composition (formulation) and does not adversely effect the stabilizing effects achieved using the methods of the invention. In one embodiment, the concentration of sugar or sugar alcohol is between about 1 mg/ml and about 150 mg/ml.

In further embodiments, the tonicity-adjusting agent is present in a concentration of from 1 mg/ml to 50 mg/ml, such as from 1 mg/ml to 7 mg/ml, from 8 mg/ml to 24 mg/ml, or from 25 mg/ml to 50 mg/ml. A pharmaceutical composition of the invention containing any of the tonicity-adjusting agents specifically mentioned above constitutes an embodiment of the invention. The use of a tonicity-adjusting agent in pharmaceutical compositions is well known to the skilled person. For convenience, reference is made to Remington: The Science and Practice of Pharmacy, 20^{th} edition, 2000.

In a still further embodiment of a pharmaceutical composition (formulation) of the invention, the formulation further comprises a chelating agent. Suitable chelating agents may be selected, for example, from salts of ethylenediaminetetraacetic acid (EDTA), citric acid, and aspartic acid, and mixtures thereof. The concentration of chelating agent will suitably be in the range from 0.1 mg/ml to 5 mg/ml, such as from 0.1 mg/ml to 2 mg/ml or from 2 mg/ml to 5 mg/ml. A pharmaceutical composition of the invention containing any of the chelating agents specifically mentioned above constitutes an embodiment of the invention. The use of a chelating agent in pharmaceutical compositions is well known to the skilled person. For convenience, reference is made to Remington: The Science and Practice of Pharmacy, 20^{th} edition, 2000.

In another embodiment of a pharmaceutical composition (formulation) of the invention, the formulation further comprises a stabilizer. The use of a stabilizer in pharmaceutical compositions is well known to the skilled person. For convenience, reference is made to Remington: The Science and Practice of Pharmacy, 20^{th} edition, 2000.

More particularly, particularly useful compositions of the invention include stabilized liquid pharmaceutical compositions whose therapeutically active components include an oligo- or polypeptide that possibly exhibits aggregate formation during storage in liquid pharmaceutical formulations. By “aggregate formation” is meant the formation of oligomers, which may remain soluble, or large visible aggregates that precipitate from the solution, as the result of a physical interaction between the oligo- or polypeptide molecules. The term “during storage” I refers to the fact that a liquid pharmaceutical composition or formulation, once prepared, is not normally administered to a subject immediately. Rather, following preparation, it is packaged for storage, whether in a liquid form, in a frozen state, or in a dried form for later reconstitution into a liquid form or other form suitable for administration to a subject. By “dried form” is meant the product obtained when a liquid pharmaceutical composition or formulation is dried by freeze-drying (i.e., lyophilization; see, for example, Williams and Polli (1984) J. Parenteral Sci. Technol. 38: 48-59), by spray-drying [see, e.g., Masters (1991) in Spray-Drying Handbook (5th edn.; Longman Scientific and Technical, Essex, U.K.), pp. 491-676; Broadhead et al. (1992) Drug Devel. Ind. Pharm. 18: 1169-1206; and Mumenthaler et al. (1994) Pharm. Res. 11: 12-20], or by air-drying [see, e.g., Carpenter and Crowe (1988) Cryobiology 25: 459-470; and Roser (1991) Biopharm. 4: 47-53]. Aggregate formation by an oligo- or polypeptide during storage of a liquid pharmaceutical composition can adversely affect biological activity of that peptide, resulting in loss of therapeutic efficacy of the pharmaceutical composition. Furthermore, aggregate formation may cause other problems, such as blockage of tubing, membranes or pumps when the oligo- or polypeptide-containing pharmaceutical composition is administered using an infusion system.

A pharmaceutical composition of the invention may further comprise an amount of an amino acid base sufficient to decrease aggregate formation by the oligo- or polypeptide during storage of the composition. By “amino acid base” is meant an amino acid, or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids for use in preparing a composition of the invention are those carrying a charged side chain, such as arginine, lysine, aspartic acid and glutamic acid. Any stereoisomer (i.e., L, D, or mixtures thereof) of a particular amino acid (e.g. methionine, histidine, arginine, lysine, isoleucine, aspartic acid, tryptophan or threonine, and mixtures thereof) or combinations of these stereoisomers, may be present in the pharmaceutical compositions of the invention so long as the particular amino acid is present either in its free base form or its salt form. In one embodiment, the L-stereoisomer of an amino acid is used. Compositions of the invention may also be formulated with analogues of these amino acids. By “amino acid analogue” is meant a derivative of a naturally occurring amino acid that brings about the desired effect of decreasing aggregate formation by the oligo- or polypeptide during storage of liquid pharmaceutical compositions of the invention. Suitable arginine analogues include, for example, aminoguanidine, ornithine and N-monoethyl-L-arginine. Suitable methionine analogues include ethionine and buthionine, and suitable cysteine analogues include S-methyl-L-cysteine. As with the amino acids per se, amino acid analogues are incorporated into compositions of the invention in either their free base form or their salt form. In a further embodiment of the invention, the amino acids or amino acid analogues are incorporated in a concentration which is sufficient to prevent or delay aggregation of the oligo- or polypeptide.

In a particular embodiment of the invention, methionine (or another sulfur-containing amino acid or amino acid analogue) may be incorporated in a composition of the invention to inhibit oxidation of methionine residues to methionine sulfoxide when the oligo- or polypeptide acting as the therapeutic agent is a peptide comprising at least one methionine residue susceptible to such oxidation. The term “inhibit” in this context refers to minimization of accumulation of methionine-oxidized species over time. Inhibition of methionine oxidation results in increased retention of the oligo- or polypeptide in its proper molecular form. Any stereoisomer of methionine (L or D) or combinations thereof can be used. The amount to be added should be an amount sufficient to inhibit oxidation of methionine residues such that the amount of methionine sulfoxide is acceptable to regulatory agencies. Typically, this means that no more than from about 10% to about 30% of forms of the oligo- or polypeptide wherein methionine is sulfoxidated are present. In general, this can be achieved by incorporating methionine in the composition such that the ratio of added methionine to methionine residues ranges from about 1:1 to about 1000:1, such as from about 10:1 to about 100:1.

In a further embodiment of the invention the formulation further comprises a stabilizer selected from high-molecular-weight polymers and low-molecular-weight compounds. Thus, for example, the stabilizer may be selected from substances such as polyethylene glycol (e.g. PEG 3350), polyvinyl alcohol (PVA), polyvinylpyrrolidone, carboxy-/hydroxycellulose and derivatives thereof (e.g. HPC, HPC-SL, HPC-L or HPMC), cyclodextrins, sulfurcontaining substances such as monothioglycerol, thioglycolic acid and 2-methylthioethanol, and various salts (e.g. sodium chloride). A pharmaceutical composition of the invention containing any of the stabilizers specifically mentioned above constitutes an embodiment of the invention.

Pharmaceutical compositions of the present invention may also comprise additional stabilizing agents which further enhance stability of a therapeutically active oligo- or polypeptide therein. Stabilizing agents of particular interest in the context of the present invention include, but are not limited to: methionine and EDTA, which protect the peptide against methionine oxidation; and surfactants, notably nonionic surfactants which protect the polypeptide against aggregation or degradation associated with freeze-thawing or mechanical shearing.

Thus, in a further embodiment of the invention, the pharmaceutical formulation comprises a surfactant, particularly a nonionic surfactant. Examples thereof include ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (Tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl-serine, phosphatidyl-choline, phosphatidyl-ethanolamine, phosphatidyl-inositol, diphosphatidyl-glycerol and sphingomyelin), derivatives of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkyl ester and alkyl ether derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium tauro-dihydrofusidate, etc.), long-chain fatty acids (e.g. oleic acid or caprylic acid) and salts thereof, acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no. [577-11-7]), docusate calcium, CAS registry no. [128-49-4]), docusate potassium, CAS registry no. [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulfonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (e.g. Tetronic's), which are tetrafunctional block copolymers derived from sequential addition of propylene oxide and ethylene oxide to ethylenediamine. The surfactant may also be selected from imidazoline derivatives and mixtures thereof. A pharmaceutical composition of the invention containing any of the surfactants specifically mentioned above constitutes an embodiment of the invention.

The use of a surfactant in pharmaceutical compositions is well-known to the skilled person. For convenience, reference is made to Remington: The Science and Practice of Pharmacy, 20^{th} edition, 2000.

Additional ingredients may also be present in a pharmaceutical composition (formulation) of the present invention. Such additional ingredients may include, for example, wetting agents, emulsifiers, antioxidants, bulking agents, metal ions, oleaginous vehicles, proteins (e.g. human serum albumin, gelatine or other proteins) and a zwitterionic species (e.g. an amino acid such as betaine, taurine, arginine, glycine, lysine or histidine). Such additional ingredients should, of course, not adversely affect the overall stability of the pharmaceutical formulation of the present invention.

Pharmaceutical compositions containing a compound according to the present invention may be administered to a patient in need of such treatment at several sites, for example at topical sites (e.g. skin and mucosal sites), at sites which bypass absorption (e.g. via administration in an artery, in a vein or in the heart), and at sites which involve absorption (e.g. in the skin, under the skin, in a muscle or in the abdomen).

Administration of pharmaceutical compositions according to the invention to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva), uretal and parenteral.

Compositions of the present invention may be administered in various dosage forms, for example in the form of solutions, suspensions, emulsions, microemulsions, multiple emulsion, foams, salves, pastes, plasters, ointments, tablets, coated tablets, rinses, capsules (e.g. hard gelatine capsules or soft gelatine capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ-transforming solutions (for example in situ gelling, in situ setting, in situ precipitating or in situ crystallizing), infusion solutions or implants.

Compositions of the invention may further be compounded in, or bound to, e,g. via covalent, hydrophobic or electrostatic interactions, a drug carrier, drug delivery system or advanced drug delivery system in order to further enhance the stability of the compound of the present invention, increase bioavailability, increase solubility, decrease adverse effects, achieve chronotherapy well known to those skilled in the art, and increase patient compliance, or any combination thereof. Examples of carriers, drug delivery systems and advanced drug delivery systems include, but are not limited to: polymers, for example cellulose and derivatives; polysaccharides, for example dextran and derivatives, starch and derivatives; poly(vinyl alcohol); acrylate and methacrylate polymers; polylactic and polyglycolic acid and block co-polymers thereof; polyethylene glycols; carrier proteins, for example albumin; gels, for example thermogelling systems, such as block co-polymeric systems well known to those skilled in the art; micelles; liposomes; microspheres; nanoparticulates; liquid crystals and dispersions thereof; L2 phase and dispersions thereof well known to those skilled in the art of phase behavior in lipid-water systems; polymeric micelles; multiple emulsions (self-emulsifying, self-microemulsifying); cyclodextrins and derivatives thereof; and dendrimers.

Compositions of the present invention are useful in the formulation of solids, semi-solids, powders and solutions for pulmonary administration of a compound of the present invention, using, for example, a metered dose inhaler, dry powder inhaler or a nebulizer, all of which are devices well known to those skilled in the art.

Compositions of the present invention are useful in the formulation of controlled-release, sustained-release, protracted, retarded or slow-release drug delivery systems. Compositions of the invention are thus of value in the formulation of parenteral controlled-release and sustained-release systems well known to those skilled in the art (both types of systems leading to a many-fold reduction in the number of administrations required).

Of particular value are controlled-release and sustained-release systems for subcutaneous administration. Without limiting the scope of the invention, examples of useful controlled release systems and compositions are those containing hydrogels, oleaginous gels, liquid crystals, polymeric micelles, microspheres, nanoparticles,

Methods for producing controlled-release systems useful for compositions of the present invention include, but are not limited to, crystallization, condensation, co-crystallization, precipitation, co-precipitation, emulsification, dispersion, high-pressure homogenisation, encapsulation, spray-drying, microencapsulation, coacervation, phase separation, solvent evaporation to produce microspheres, extrusion and supercritical fluid processes. General reference is made in this context to Handbook of Pharmaceutical Controlled Release (Wise, D. L., ed. Marcel Dekker, New York, 2000), and Drugs and the Pharmaceutical Sciences, vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, for example a syringe in the form of a pen device. Alternatively, parenteral administration can be performed by means of an infusion pump. A further option is administration of a composition of the invention which is a liquid (typically aqueous) solution or suspension in the form of a nasal or pulmonary spray. As a still further option, a pharmaceutical composition of the invention can be adapted to transdermal administration (e.g. by needle-free injection or via a patch, such as an iontophoretic patch) or transmucosal (e.g. buccal) administration.

The term “stabilized formulation” refers to a formulation with increased physical stability, increased chemical stability or increased physical and chemical stability. The term “physical stability” in the context of a formulation containing an oligo- or polypeptide refers to the tendency of the peptide to form biologically inactive and/or insoluble aggregates as a result of exposure to thermo-mechanical stresses and/or interaction with interfaces and surfaces that are destabilizing, such as hydrophobic surfaces and interfaces. Physical stability of aqueous protein formulations is evaluated by means of visual inspection and/or turbidity measurements after exposing the formulation, filled in suitable containers (e.g. cartridges or vials), to mechanical/physical stress (e.g. agitation) at different temperatures for various time periods. Visual inspection of formulations is performed in a sharp focused light with a dark background. The turbidity of a formulation is characterized by a visual score ranking the degree of turbidity, for instance on a scale from 0 to 3 (in that a formulation showing no turbidity corresponds to a visual score 0, whilst a formulation showing visual turbidity in daylight corresponds to visual score 3). A formulation is normally classified physically unstable with respect to aggregation when it shows visual turbidity in daylight. Alternatively, the turbidity of a formulation can be evaluated by simple turbidity measurements well-known to the skilled person. Physical stability of aqueous oligo- or polypeptide formulations can also be evaluated by using a spectroscopic agent or probe of the conformational status of the peptide. The probe is preferably a small molecule that preferentially binds to a non-native conformer of the oligo- or polypeptide. One example of a small-molecular spectroscopic probe of this type is Thioflavin T. Thioflavin T is a fluorescent dye that has been widely used for the detection of amyloid fibrils. In the presence of fibrils, and possibly also other configurations, Thioflavin T gives rise to a new excitation maximum at about 450 nm, and enhanced emission at about 482 nm when bound to a fibril form. Unbound Thioflavin T is essentially non-fluorescent at the wavelengths in question.

Other small molecules can be used as probes of the changes in peptide structure from native to non-native states. Examples are the “hydrophobic patch” probes that bind preferentially to exposed hydrophobic patches of a polypeptide. The hydrophobic patches are generally buried within the tertiary structure of a polypeptide in its native state, but become exposed as it begins to unfold or denature. Examples of such small-molecular, spectroscopic probes are aromatic, hydrophobic dyes, such as antrhacene, acridine, phenanthroline and the like. Other spectroscopic probes are metal complexes of amino acids, such as cobalt complexes of hydrophobic amino acids, e.g. phenylalanine, leucine, isoleucine, methionine, valine, or the like.

The term “chemical stability” of a pharmaceutical formulation as used herein refers to chemical covalent changes in oligo- or polypeptide structure leading to formation of chemical degradation products with potentially lower biological potency and/or potentially increased immunogenicity compared to the original molecule. Various chemical degradation products can be formed depending on the type and nature of the starting molecule and the environment to which it is exposed. Elimination of chemical degradation can most probably not be completely avoided and gradually increasing amounts of chemical degradation products may often be seen during storage and use of oligo- or polypeptide formulations, as is well known to the person skilled in the art. A commonly encountered degradation process is deamidation, a process in which the side-chain amide group in glutaminyl or asparaginyl residues is hydrolysed to form a free carboxylic acid. Other degradation pathways involve formation of higher molecular weight transformation products wherein two or more molecules of the starting substance are covalently bound to each other through transamidation and/or disulfide interactions, leading to formation of covalently bound dimer, oligomer or polymer degradation products (see, e.g., Stability of Protein Pharmaceuticals, Ahern. T. J. & Manning M. C., Plenum Press, New York 1992). Oxidation (of for instance methionine residues) may be mentioned as another variant of chemical degradation. The chemical stability of a formulation may be evaluated by measuring the amounts of chemical degradation products at various time-points after exposure to different environmental conditions (in that the formation of degradation products can often be accelerated by, e.g., increasing temperature). The amount of each individual degradation product is often determined by separation of the degradation products depending on molecule size and/or charge using various chromatographic techniques (e.g. SEC-HPLC and/or RP-HPLC).

Hence, as outlined above, a “stabilized formulation” refers to a formulation with increased physical stability, increased chemical stability, or increased physical and chemical stability. In general, a pharmaceutical composition (formulation) must be stable during use and storage (in compliance with recommended use and storage conditions) until the expiry date is reached.

A pharmaceutical composition (formulation) of the invention should preferably be stable for more than 2 weeks of usage and for more than two years of storage, more preferably for more than 4 weeks of usage and for more than two years of storage, desirably for more than 4 weeks of usage and for more than 3 years of storage, and most preferably for more than 6 weeks of usage and for more than 3 years of storage.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

Headings and sub-headings are used herein for convenience only, and should not be construed as limiting the invention in any way.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The citation and incorporation of patent documents herein is done for convenience only, and does not reflect any view of the validity, patentability and/or enforceability of such patent documents.

The present invention includes all modifications and equivalents of the subject matter recited in the claims appended hereto, as permitted by applicable law.

### EXAMPLES

### List of Abbreviations Employed

AcOH acetic acid
BCMA [bis(carboxymethyl)amino]acetyl
Bn benzyl
BSA bovine serum albumin
DBU 1,8-diazabicyclo[5.4.0]undec-7-ene
DCM dichloromethane
Dde 1-(4,4-dimethyl-2,6-dioxocyclohex-1-ylidene)ethyl
DIC N,N-diisopropylcarbodiimide
DIPEA ethyldiisopropylamine
DMAP 4-(dimethylamino)pyridine
DMF N,N-dimethylformamide
DMSO dimethylsulfoxide
EDC 1-ethyl-3-(3-dimethylaminopropyl)carbodiimide hydrochloride
EDT 1,2-ethanedithiol
EGTA glycol-bis(2-aminoethylether)-N,N,N′,N′-tetraacetic acid (ethyleneglycol tetraacetic acid)
eq equivalents
FCS fetal calf serum
Fmoc 9H-fluoren-9-ylmethyloxycarbonyl
Fmoc SPPS Fmoc-based solid-phase peptide synthesis
HBTU 2-(1H-benzotriazol-1-yl-)-1,1,3,3-tetramethyluronium hexafluorophosphate
HEPES 2-[4-(2-hydroxyethyl)-piperazin-1-yl]ethanesulfonic acid
HFIP 1,1,1,3,3,3-hexafluoro-2-propanol
HOAt 1-hydroxy-7-aza-benzotriazole
HOBt 1-hydroxybenzotriazole
HSA human serum albumin
IBMX 3-isobutyl-1-methylxanthine
ivDde 1-(4,4-dimethyl-2,6-dioxocyclohex-1-ylidene)-3-methylbutyl
MC1 melanocortin receptor subtype 1 (also denoted melanocortin receptor 1)
MC2 melanocortin receptor subtype 2 (also denoted melanocortin receptor 2)
MC3 melanocortin receptor subtype 3 (also denoted melanocortin receptor 3)
MC4 melanocortin receptor subtype 4 (also denoted melanocortin receptor 4)
MC5 melanocortin receptor subtype 5 (also denoted melanocortin receptor 5)
MeCN acetonitrile
MeOH methanol
min minutes
α-MSH α-form of melanocyte-stimulating hormone
Mtt 4-methyltrityl
MTX methotrexate
NEt₃ triethylamine
NMP N-methylpyrrolidin-2-one
OPhiPr 2-phenylisopropyloxy=1-methyl-1-phenyl-ethyloxy
OSu ester 2,5-dioxo-pyrrolidin-1-yl ester
PBS phosphate-buffered saline
PEI polyethyleneimine
PhiPr 2-phenylisopropyl=1-methyl-1-phenyl-ethyl
PyBOP (benzotriazol-1-yloxy)trispyrrolidinophosphonium hexafluorophosphate
TFA trifluoroacetic acid
THF tetrahydrofuran
TIPS triisopropylsilane
TSTU O—(N-succinimidyl)-N,N,N′,N′-tetramethyluronium tetrafluoroborate
trityl triphenylmethyl
Trt triphenylmethyl
HPLC ultra performance liquid chromatography

All compounds of the present invention can be synthesized by those skilled in the art using standard coupling and deprotection steps. Non-standard procedures and syntheses of special building blocks are described below. A description of necessary tools and synthetic methods including standard abbreviations for peptide synthesis can be found in “Reagents for Peptide and High-Throughput Synthesis”, 2004/5 Catalogue, Novabiochem.

### General Procedures

### Automated Peptide Synthesis

Peptides can be synthesized according to the Fmoc strategy on an Applied Biosystems ABI-433A peptide synthesizer on a 0.25 mmol or 1.0 mmol scale using the manufacturer supplied FastMoc UV protocols which employ the Fmoc protected amino acid (4 equivalents), HOBt (4 equivalents), HBTU (4 equivalents) and DIPEA (8 equivalents) in NMP, and UV monitoring of the deprotection of the Fmoc protection group. Piperidine in NMP is used for deprotection of the Fmoc protected amino acids.

Fmoc-based automated peptide synthesis can also be done on a CEM Liberty microwave peptide synthesizer (HOAt/DIC-mediated couplings without base and without preactivation; Fmoc removal with piperidine in NMP), or on a Protein Technologies Prelude peptide synthesizer (HOAt/DIC-mediated couplings in the presence of 2,4,6-collidine and without preactivation; Fmoc removal with piperidine in NMP).

Cleavage from the Resin and Side-Chain Deprotection

After completed solid-phase peptide synthesis, the resin is extensively washed with DCM. The resin is shaken for 1.5-3 h with a cleavage cocktail consisting of TFA (≧90%) and suitable cation scavengers (≦10%). The mixture is filtered and the filtrate is collected. The resin is washed with TFA/DCM and the filtrate is collected. The combined filtrate solution is concentrated under reduced pressure. The peptide is precipitated with excess diethylether, collected by centrifugation or filtration, washed with diethylether and dried. For the cleavage of peptides containing a tetrazol-5-yl group or trityl-protected mercapto groups, Procedure 7 (see below) is recommended. For the cleavage of disulfide peptides and peptides without tetrazole, Procedure 8 (see below) is recommended. Cleavage of disulfide peptides containing a tetrazol-5-yl group is possible similarity to Procedure 8, but a larger volume of TFA/TIPS/water 95:2.5:2.5, should be used, in order to minimize capture of tert-butyl ions by the tetrazole ring.

### Purification and Quantification

The crude peptide is dissolved in a suitable mixture of water and MeCN or N-methylformamide and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or HPLC. Fractions containing the pure target peptide are mixed and concentrated under reduced pressure. The resulting solution is analyzed (HPLC, LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying for three days affords the peptide trifluoroacetate as a white solid.

### Special Procedures

Procedure 1: Removal of ivDde from a Resin-Bound Amino Group

The resin is washed 3 times for 20 min with a freshly prepared solution of 3% hydrazine hydrate in NMP. The resin is washed with NMP, 5% HOBt in NMP and 5 times with NMP.

Procedure 2: Removal of Trityl from a Resin-Bound α-Amino Group

The resin (0.25 mmol) is washed 5 times with DCM and then shaken with a solution of 2% TFA and 3% TIPS in DCM (15 ml) for 3 min and drained. This step is repeated another 7 times, or more times, if the filtrate is still yellow. The resin is washed with DCM (3×15 ml), 5% DIPEA in DCM (2×15 ml), NMP (10 ml) and DCM (5×15 ml).

Procedure 3: Removal of Mtt from a Resin-Bound Amino Group

The resin is washed 5 times with DCM and then 2 times for 15 min with HFIP/DCM/TIPS 70:28:3. The resin is washed 2 times with DCM (2×10 ml), 2 times with 5% DIPEA in DCM, once with NMP and 5 times with DCM.

### Procedure 4: Solid-Phase Lactam Cyclization

The peptide resin (0.25 mmol) containing an Mtt-protected amino group and a phenylisopropyl-protected carboxy group is washed 5 times with DCM and then shaken with a solution of 2% TFA and 3% TIPS in DCM (10 ml) for 3 min and drained. This step is repeated another 7 times, or more times, if the filtrate is still yellow. The resin is washed with DCM (3×10 ml), 5% DIPEA in DCM (2×10 ml) and NMP (3×10 ml). A solution of HOBt (101 mg, 0.75 mmol) and PyBOP (390 mg, 0.75 mmol) in NMP/DCM 1:1 (8 ml) is added to the resin, followed by addition of DIPEA (0.257 ml, 1.50 mmol). The mixture is shaken overnight. The resin is washed with NMP (5×10 ml).

Procedure 5: Solid-Phase Reductive Alkylation with Glyoxalic Acid

A solution of glyoxalic acid monohydrate (258 mg, 2.8 mmol) in MeOH (1.8 ml) and NMP (6.6 ml) is added to the resin (0.25 mmol). HOAc (0.160 ml, 2.8 mmol) and a freshly prepared solution of sodium cyanoborohydride (201 mg, 3.2 mmol) in MeOH (1.8 ml) and NMP (6.6 ml) is added. The mixture is shaken overnight. The resin is washed with NMP (5×10 ml).

Procedure 6: Solid-Phase Acylation with bis(tert-butoxycarbonylmethyl)aminoacetic Acid

A solution of bis(tert-butoxycarbonylmethyl)aminoacetic acid (303 mg, 1.0 mmol; available by the synthetic procedure described below) in NMP (8 ml) is added to the resin (0.25 mmol). PyBOP (520 mg, 1.0 mmol) and DIPEA (0.342 ml, 2.0 mmol) are added. The mixture is shaken for 2 h. The resin is washed 5 times with NMP.

Procedure 7: Cleavage from the Resin and Side-Chain Deprotection (Recommended for Peptides Containing tetrazol-5-yl or Peptides with trityl-protected Mercapto Groups)

The resin (0.25 mmol) is washed 5 times with DCM and then with DCM/EDT/TIPS 90:5:5 (10 ml). After filtration, the resin is shaken for 2 h with a premixed solution of TFA/EDT/TIPS/water 90:5:4:1 (10 ml). The liquid is collected by filtration. The resin is washed with DCM/TFA 2:1 (15 ml) and the filtrate is collected. The combined collected filtrate is concentrated under reduced pressure and thereafter treated with diethylether (40 ml). The precipitated peptide is collected by centrifugation and washed with diethylether (2×40 ml) to give a white solid.

Procedure 8: Cleavage from the Resin and Side-Chain Deprotection (Recommended for Peptides without tetrazol-5-yl and Peptides Containing Disulfide Bonds)

The resin (0.25 mmol) is washed 5 times with DCM and then shaken for 2 h with a premixed solution of TFA/TIPS/water 95:2.5:2.5 (10 ml). The liquid is collected by filtration. The resin is washed with DCM/TFA 2:1 (15 ml) and the filtrate is collected. The combined collected filtrate is concentrated under reduced pressure and thereafter treated with diethylether (40 ml). The precipitated peptide is collected by centrifugation and washed with diethylether (2×40 ml) to give a white solid.

### Procedure 9: Disulfide Cyclization in Solution

The crude peptide TFA salt (obtained from 0.25 mmol of resin) is dissolved in MeOH/water 95:5 (75 ml). A solution of iodine (254 mg, 1.0 mmol) in DCM/MeOH 1:4 (5 ml) is added slowly over 5 min to the peptide solution with stirring. The resulting dark brown mixture is stirred for 30 min and then quenched with a solution of ascorbic acid (177 mg, 1.0 mmol) in water to give a colourless solution. The mixture is concentrated under reduced pressure. The peptide is purified by preparative HPLC.

### Procedure 10: Disulfide Cyclization on Resin

The peptide resin (0.25 mmol) containing trityl-protected mercapto groups is shaken with a solution of iodine (632 mg, 2.5 mmol) in NMP (10 ml) for 1 h and then drained. The resin is washed with NMP (5×10 ml) and then shaken for 10 min with a solution of ascorbic acid (132 mg, 0.75 mmol) in water (0.75 ml) and NMP (10 ml). The resin is washed with NMP/MeOH 4:1 (4×10 ml), NMP (3×10 ml) and DCM (6×10 ml).

Procedure 11: Solution-Phase Reductive Alkylation with Glyoxalic Acid

The crude peptide (obtained from 0.25 mmol of resin) is dissolved in a mixture of N-methylformamide (5 ml), MeOH (8.5 ml) and 0.2 M citrate buffer pH 4.5 (9.0 ml, 1.8 mmol; preparation of the buffer: 0.2 mol of citric acid and 0.35 mol of NaOH dissolved in one liter of water). Glyoxalic acid monohydrate (0.424 g, 4.6 mmol) and a freshly prepared solution of sodium cyanoborohydride (0.163 g, 2.6 mmol) in MeOH (3 ml) are added. The mixture is stirred for 24 h. The peptide is purified by preparative HPLC.

Procedure 12: Solid-Phase Acylation with Fmoc-Dab(Mtt)-OH

A solution of Fmoc-Dab(Mtt)-OH (0.746 g, 1.25 mmol) and HOBt (0.169 g, 1.25 mmol) in NMP (5 ml) and DCM (2.5 ml) is added to the resin (0.25 mmol), followed by addition of PyBOP (0.650 g, 1.25 mmol) and DIPEA (0.428 ml, 2.5 mmol). The mixture is shaken overnight and then filtered. The resin is washed with NMP (5×10 ml) and DCM (5×10 ml).

Procedure 13: Acylation with bis(tert-butoxycarbonylmethyl)aminoacetic Acid and Subsequent Removal of tert-butyl

In a small test tube, bis(tert-butoxycarbonylmethyl)aminoacetic acid (49 mg, 0.160 mmol; available by the synthetic procedure described above) and TSTU (48 mg, 0.160 mmol) are mixed with NMP (1.5 ml). DIPEA (0.066 ml, 0.384 mmol) is added. The tube is capped and shaken for 2 h. The resulting OSu ester solution is then used for the following acylation. In a test tube, the crude peptide TFA salt (obtained from 0.25 mmol of resin) is dissolved in NMP (3 ml). DIPEA (0.072 ml, 0.420 mmol) is added. To the resulting solution, the OSu ester solution is added. The tube is capped and shaken for 3 h. The reaction mixture is dropped into diethylether (40 ml). The resulting precipitate is collected by centrifugation and washed again with diethylether (40 ml). The liquid phase is removed by centrifugation. To the resulting residue, a premixed solution of TIPS (0.5 ml) in TFA (9.5 ml) is added. The resulting solution is stirred for 90 min and then concentrated to give a liquid residue (appr. 2 ml). The liquid is treated with diethylether (40 ml) to give a white precipitate. The precipitate is collected by centrifugation, washed again with diethylether (40 ml) and dried to give a white solid.

### Preparation of bis(tert-butoxycarbonylmethyl)aminoacetic acid

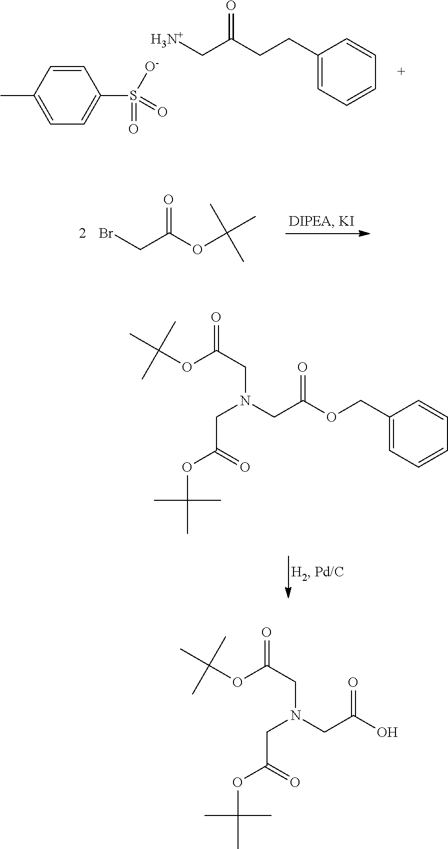

Bromoacetic acid tert-butyl ester (313.3 ml, 2.16 mol), DIPEA (179.5 ml, 1.08 mol) and potassium iodide (25.9 g, 216 mmol) were subsequently added to a solution of glycine benzyl ester p-methylbenzenesulfonic acid salt (72.95 g, 216 mmol) in DMF (730 ml). The resulting mixture was stirred at room temperature for 3 days under nitrogen. The solvent was evaporated in vacuo; the residue was diluted with DCM (300 ml) and 5% aqueous solution of sodium carbonate (300 ml). The organic phase was washed with another portion of 5% aqueous solution of sodium carbonate (300 ml) and dried (Na₂SO₄). The solvent was evaporated in vacuo. The residue was filtered through silica gel (200 g, Fluka 60) using hexanes/ethylacetate mixture (2:1). After removal of solvent in vacuo the purification process was repeated twice. The solvent was evaporated to give bis(tert-butoxycarbonylmethyl)aminoacetic acid benzyl ester as a viscous yellow liquid.

Yield: 58.19 g (68%)

¹H NMR spectrum (300 MHz, CDCl₃): δ 7.49-7.38 (m, 5H); 5.15 (s, 2H); 3.69 (s, 2 H); 3.54 (s, 4H); 1.44 (s, 18H).

Palladium on carbon (10%, 15 g) was added to a degassed solution of bis(tert-butoxycarbonylmethyl)aminoacetic acid benzyl ester (58.19 g, 148.8 mmol) in methanol (440 ml) and the reaction mixture was hydrogenated at 435 psi for 24 hrs. The mixture was filtered through a pad of Celite. The procedure was repeated three additional times. The filtrates were combined and evaporated in vacuo to give the title compound as a yellow solid. The residue was recrystallized four times from hexanes at −20° C. The solid was filtered off and dried in vacuo to give bis(tert-butoxycarbonylmethyl)aminoacetic acid.

Yield: 25.7 g (57%)

Melting point: 76-82° C.

¹H NMR spectrum (300 MHz, CDCl₃): δ 3.48 (s, 2H); 3.47 (s, 4H); 1.47 (s, 18H).

### Preparation of Fmoc-Lys(bis(tert-butoxycarbonylmethyl))-OH

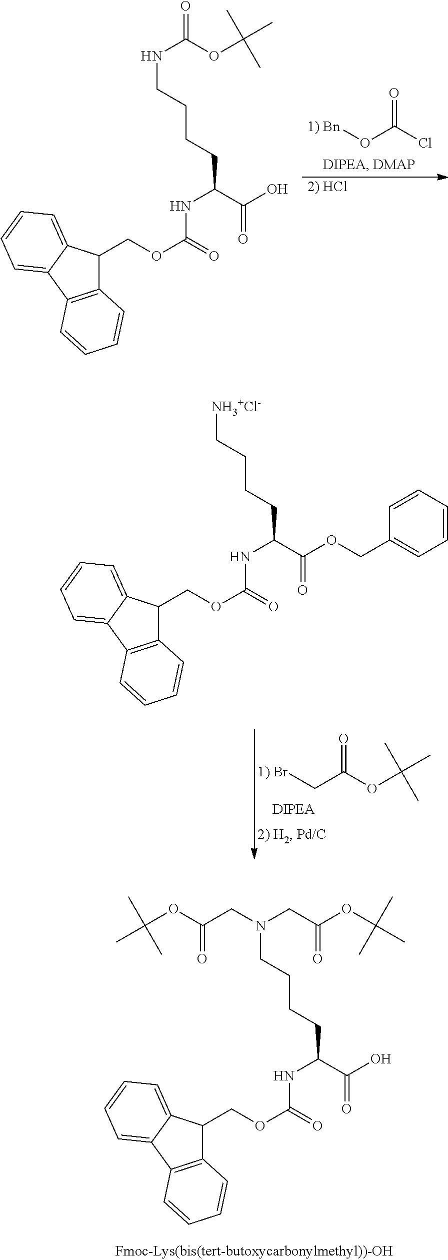

A solution of benzyl chloroformate (8.8 ml, 61.3 mmol) in DCM (50 mL) was added dropwise to a stirred solution of Fmoc-Lys(Boc)-OH (50 g, 53.6 mmol), DIPEA (27 ml, 78 mmol) and DMAP (650 mg, 5.3 mmol) in DCM (250 mL) at 0° C. The mixture was stirred at 0° C. for 24 hrs; then it was washed with 5% aqueous citric acid and water (200 mL). The organic layer was dried over anhydrous sodium sulfate and evaporated in vacuo. The residue was taken up in DCM (30 mL), filtered (S3) and purified by column chromatography (silica gel, hexanes/ethyl acetate 3:1). The fractions containing the product were evaporated in vacuo. The resulting solid was reevaporated from ethyl acetate to give Fmoc-Lys(Boc)-OBn as white amorphous powder.

Yield: 49.0 g (82%).

¹H NMR spectrum (300 MHz, CDCl₃): δ 7.79 (d, J=7.3 Hz, 2H); 7.62 (d, J=7.3 Hz, 2H); 7.48-7.29 (m, 9H); 5.44 (d, 1H); 5.21 (dd, 2H); 4.62-4.33 (m, 3H); 4.24 (t, 1H); 3.20-2.97 (m, 2H); 1.97-1.61 (m, 2H); 1.57-1.38 (m, 11H); 1.41-1.15 (m, 2H).

The above Fmoc-Lys(Boc)-OBn (31.32 g, 54 mmol) was dissolved in anhydrous DCM (60 mL), and solution of hydrogen chloride in dioxane (2.1 M, 205 mmol, 55 mL) was added. The reaction mixture was stirred at room temperature for 10 hrs before removal of the solvent under reduced pressure. The solid residue was dried on air. This crude product was used without further purification. LC/MS analysis proved a completion of the reaction. The reaction was done in two batches.

Crude Fmoc-Lys-OBn HCl salt (50.8 g, 102 mmol) was dissolved in dry DMF (250 mL), and DIPEA (87 ml, 510 mmol), and tert-butyl bromoacetate (45 mL, 306 mmol) were added to the solution. The mixture was stirred at room temperature for 3 hrs, and DMF was removed under reduced pressure (at 50° C.). The residue was suspended in water (500 mL) and extracted with DCM (3×500 mL). The organic layer was dried over anhydrous sodium sulfate and evaporated in vacuo. The residue was purified by column chromatography (silica gel, gradient elution hexanes/ethyl acetate 9:1 to 7:3) to give Fmoc-Lys(bis(tert-butoxycarbonylmethyl))-OBn as pale yellow oil. Chromatography of mixed fractions was repeated.

Yield: 54.24 g (77%).

¹H NMR spectrum (300 MHz, CDCl₃): δ 7.76 (d, J=7.2 Hz, 2H); 7.60 (d, J=6.6 Hz, 2H); 7.45-7.23 (m, 9H); 5.51 (d, 2H); 5.17 (dd, 2H); 4.44-4.30 (m, 2H); 4.20-3.95 (m, 2H); 3.41 (s, 4H); 2.65-2.58 (m, 3H), 1.96-1.30 (m, 6H), 1.45 (s, 18H).

Fmoc-Lys(bis(tert-butoxycarbonylmethyl))-OBn (54.24 g, 79 mmol) was dissolved in methanol (500 mL). Palladium on carbon (5 wt %, 3.35 g) was added to the solution. The suspension was stirred under hydrogen atmosphere at room temperature. After 3 hrs, the mixture was filtered through Celite and the filtrate was concentrated. The crude product was purified by flash column chromatography (silica gel, DCM/methanol 95:5) to afford the title compound Fmoc-Lys(bis(tert-butoxycarbonylmethyl))-OH as white solid.

Yield: 31.4 g (67%).

Melting point: 51-52° C.

¹H NMR spectrum (300 MHz, CDCl₃): δ 7.76 (d, J=7.3 Hz, 2H); 7.60 (d, J=6.6 Hz, 2H); 7.39 (t, J=7.3 Hz, 2H); 7.30 (t, J=7.4 Hz, 2H); 5.67 (d, J=7.2 Hz, 1H); 4.31-4.53 (m, 3H); 4.17-4.26 (m, 1H); 3.54 (s, 1H); 2.64-2.91 (m, 2H); 1.44 (s, 18H), 1.19-1.99 (m, 6H).

### Preparation of (S)-2-Fmoc-amino-3-{2-[bis(tert-butoxycarbonylmethyl)amino]aetylamino}propionic acid

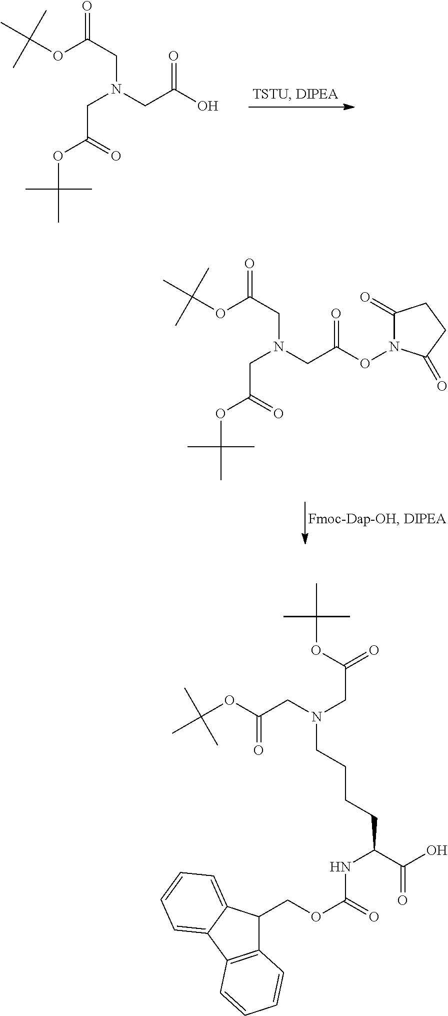 [SMILES: CC(C)(C)OC(=O)CN(CC(=O)O)CC(=O)OC(C)(C)C ; CC(C)(C)OC(=O)CN(CC(=O)ON1C(=O)CCC1=O)CC(=O)OC(C)(C)C ; CC(C)(C)OC(=O)CN(CCCC[C@H](NC(=O)OCC1c2ccccc2-c2ccccc21)C(=O)O)CC(=O)OC(C)(C)C]

To bis(tert-butoxycarbonylmethyl)aminoacetic acid (1.0 g, 3.3 mmol) in dry THF (60 ml) was added DIPEA (0.84 ml, 4.9 mmol) and TSTU (1.78 g, 4.9 mmol) and the mixture was stirred for 3 days at room temperature. The solvent was removed in vacuo and the residue was divided by a mixture of ethylacetate (75 ml) and 5% citric acid in water (75 ml). The organic phase was dried over Na₂SO₄ and the solvent was removed in vacuo. The resulting crude bis(tert-butoxycarbonylmethyl)aminoacetic acid 2,5-dioxo-pyrrolidin-1-yl ester was pure enough for further synthesis.

To (S)-3-amino-2-(9H-fluoren-9-ylmethoxycarbonylamino)propionic acid (Fmoc-Dap-OH; 1.0 g, 3.06 mmol) in THF (50 mL) was added DIPEA (0.52 mL, 3.06 mmol) and bis(tert-butoxycarbonylmethyl)aminoacetic acid 2,5-dioxo-pyrrolidin-1-yl ester (2.43 g, 6.12 mmol) and the mixture was stirred for 3 hours before the solvent was removed in vacuo. The crude product was subjected to preparative HPLC to give 1.2 g (64% yield) of (S)-2-Fmoc-amino-3-{2-[bis(tert-butoxycarbonylmethyl)amino]acetylamino}propionic acid.

### (2-{2-[16-(Tetrazol-5-yl)hexadecanoylamino]ethoxy}ethoxy)acetic acid

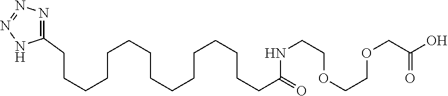 [SMILES: O=C(O)COCCOCCNC(=O)CCCCCCCCCCCCCCCc1nnn[nH]1]

2-Chlorotritylchloride resin 100-200 mesh (495 g, 495 mmol) was left to swell in dry DCM (2500 ml) for 20 min. A solution of Fmoc-8-amino-3,6-dioxaoctanoic acid (173.4 g, 450 mmol) and DIPEA (298 ml, 1.7 mol) in dry DCM (320 ml) was added and the mixture was shaken for 3.5 h. The resin was filtered and treated with a solution of DIPEA (160 ml) in MeOH/DCM mixture (2:8, 1000 ml, 2×5 min). The resin was then washed with DMF (2×1200 ml), DCM (2×1500 ml) and DMF (3×1200 ml). Fmoc group was removed by treatment with 20% piperidine in DMF (1×30 min, 1×60 min, 2×1000 ml). The resin was washed with DMF (3×1200 ml), 2-propanol (2×1000 ml) and DCM (1×2500 ml, 2×1500 ml). Resin loading was determined as 0.54 mmol/g. A solution of 16-(tetrazol-5-yl)hexadecanoic acid (142 g, 439 mmol; available by the synthetic procedure described in WO 2007/009894), O-(6-chloro-1H-benzotriazole-1-yl)-N,N,N′,N′-tetramethyluronium tetrafluoroborate (156.1 g, 439 mmol) and DIPEA (497 ml, 2.85 mol) in DMF (430 ml) and DCM (210 ml) were added to the resin and the mixture was shaken for 23 h. The resin was drained and washed with DMF (3×1500 ml), DCM (2×1800 ml), methanol (3×1500 ml) and DCM (1×2500 ml, 6×1600 ml).

The product was cleaved from the resin by treatment with TFA/water/TIPS 95:2.5:2.5 (1500 ml) for 3 h; then the resin was filtered off and washed with DCM/TFA mixture (9:1, 2×1500 ml), DCM (5×1000 ml) and chloroform (3×1000 ml). The volatiles were removed under reduced pressure. The residue was triturated with diethyl ether (2000 ml). The solid was filtered, washed with diethyl ether (6×300 ml) and dried in vacuo to give the title compound as beige solid.

Yield: 105.21 g (66%).

Melting point: 102-104° C.

R_{F} (SiO₂, DCM/methanol/acetic acid 89:10:1): 0.20.

¹H NMR spectrum (300 MHz, AcOD-d₄, δ): 4.27 (s, 2H); 3.85-3.77 (m, 2H); 3.77-3.72 (m, 2H); 3.69 (t, J=5.0 Hz, 2H); 3.51 (t, J=4.8 Hz, 2H); 3.07 (t, J=7.6 Hz, 2H); 2.34 (t, J=7.6 Hz, 2 H); 1.84 (m, 2H); 1.67 (m, 2H); 1.39 (brs, 4H); 1.34 (brs, 18H).

LC-MS purity (ELSD): 100%.

LC-MS Rt (Sunfire 4.6 mm×100 mm, acetonitrile/water 75:25+0.1% FA): 6.25 min.

LC-MS m/z: 470.2 (M+H)⁺.

### Preparation of heptadecanedioic acid mono-tert-butyl ester

### 2-Methoxycarbonyl-heptadecanedioic acid 1-methyl ester

Sodium (1.11 g, 48.2 mmol) was dissolved in dry methanol (30 ml) and heated to 50° C. Dimethyl malonate (5.87 ml, 51.4 mmol) was added over 15 min. The mixture was heated to reflux and a suspension of 15-bromopentadecanoic acid (5 g, 15.6 mmol) in dry methanol (50 ml) was added over 45 min. The resulting mixture was refluxed for another 30 min. After cooling to RT, water was added and the mixture was concentrated. Water was added to the residue and made alkaline with 1 N NaOH and extracted with ether (1×50 ml). The aqueous layer was acidified with 1N HCl and extracted with ether (3×30 ml). The combined organic layers were dried (Na₂SO₄) and concentrated to give the title compound in 98% (5.7 g) yield.

¹H-NMR (CDCl₃): δ 1.2-1.35 (m, 22H), 1.65 (pent, 2H), 1.90 (m, 2H), 2.34 (t, 2H), 3.37 (t, 1H), 3.71 ppm (s, 6H).

### Heptadecanedioic Acid

2-Methoxycarbonyl-heptadecanedioic acid 1-methyl ester (4.63 g, 12.4 mmol) was dissolved in 20% aqueous KOH (15 ml) by heating. The resulting solution was refluxed for 2.5 h. The cold reaction mixture was carefully concentrated. The residue was suspended in water (30 ml) on an ice bath and acidified with 10% aqueous HCl. The resulting slurry was refluxed for 2 h. After cooling the precipitate was isolated by filtration and dried over night in vacuo. The compound was decarboxylated by heating under stirring at 140° C. for 2 h. (The reaction should be followed, heating to 180° might be necessary). The crude product (4.0 g, 100%) was used without further purification.

¹H-NMR (DMSO-d₆): δ 1.22 (br s, 22H) 1.47 (m, 4H), 2.18 (t, 4H).

### Heptadecanedioic acid mono-tert-butyl ester

Crude heptadecanedioic acid (0.99 g, 3.3 mmol) was dissolved in toluene (15 ml) at 115° C. N,N-dimethylformamide di-tert-butylacetale (0.79 ml, 3.3 mmol) was added dropwise over 10 min. After refluxing for 1 h, more N,N-dimethylformamide di-tert-butylacetale (0.79 ml) was added over 10 min. After refluxing for another 1 h, a last eq of N,N-dimethylformamide di-tert-butylacetale (0.79 ml) was added over 10 min. Reflux was continued for 1 h. On cooling to RT a precipitate appeared, this was filtered off (diacid). The mother liqueour was extracted with water (25 ml) and DCM (25 ml). The organic layer was dried and concentrated. The residue was purified by flash chromatography using DCM/MeOH 15:1 as eluent. The title compound was isolated in 33% yield (0.330 g).

¹H-NMR (DMSO-d₆): δ 1.22 (br s, 22H), 1.39 (s, 9H), 1.47 (m, 4H), 2.16 (t, 2H), 2.19 ppm (t, 2H).

### Preparation of 16-(3-carboxy-propane-1-sulfonylamino)-16-oxo-hexadecanoic acid tert-butyl ester

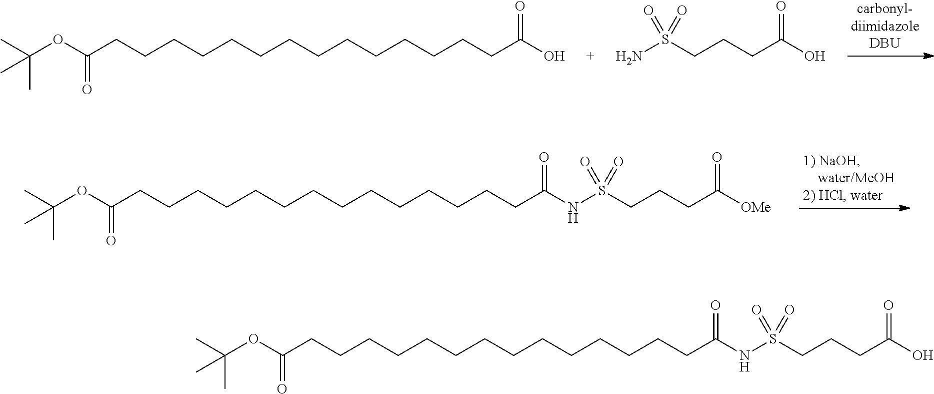 [SMILES: CC(C)(C)OC(=O)CCCCCCCCCCCCCCC(=O)O ; NS(=O)(=O)CCCC(=O)O ; COC(=O)CCCS(=O)(=O)NC(=O)CCCCCCCCCCCCCCC(=O)OC(C)(C)C ; CC(C)(C)OC(=O)CCCCCCCCCCCCCCC(=O)NS(=O)(=O)CCCC(=O)O]

Hexadecanedioic acid mono-tert-butyl ester (5.14 g, 15.0 mmol; available by the synthetic procedure described in: U. Widmer, Synthesis 1983, 135) was dissolved in DCM (30 ml) and MeCN (30 ml). Carbonyldiimidazole (2.51 g, 15.45 mmol) was added and the mixture was stirred for 2 h. A solution of (4-sulfamoyl)butyric acid methyl ester (2.72 g, 15.0 mmol) in DCM (30 ml) was added, followed by addition of DBU (2.69 ml, 18 mmol). The mixture was stirred overnight and then concentrated under reduced pressure. The resulting residue was treated with 0.2 M aqueous citrate buffer pH 4.5 (preparation of the buffer: 0.2 mol of citric acid and 0.35 mol of NaOH dissolved in one liter of water). After 20 min, the resulting precipitate was collected by filtration and washed with water (150 ml).

This product was dissolved in MeOH (70 ml) and THF (20 ml). 1M aqueous NaOH (13 ml, 13 mmol) was slowly added and the mixture was stirred. After 40 min, a new portion of 1M aqueous NaOH (14.3 ml, 14.3 mmol) was slowly added. The mixture was stirred overnight and then poured into a mixture of water (150 ml) and 0.2 M aqueous citrate buffer pH 4.5 (150 ml). After 1 h, the resulting precipitate was collected by filtration, washed with water (100 ml) and dried to give the crude title compound. Recrystallization from acetone (300 ml) afforded 2.44 g (33% yield) of 16-(3-carboxy-propane-1-sulfonylamino)-16-oxo-hexadecanoic acid tert-butyl ester.

¹H NMR (DMSO-d6) δ 1.23 (m, 20H), 1.39 (s, 9H), 1.48 (m, 4H), 1.84 (m, 2H), 2.16 (t, J 7 Hz, 2H), 2.24 (t, J 7 Hz, 2H), 2.38 (t, J 7 Hz, 2H), 3.37 (m, partially overlapping with water peak at 3.33 ppm).

Possible Reaction Scheme for the Synthesis of Compounds According to Formula I with Lactam Macrocycle, Z³ Containing an Alkylated Amino Group and R⁶═NH-alkyl

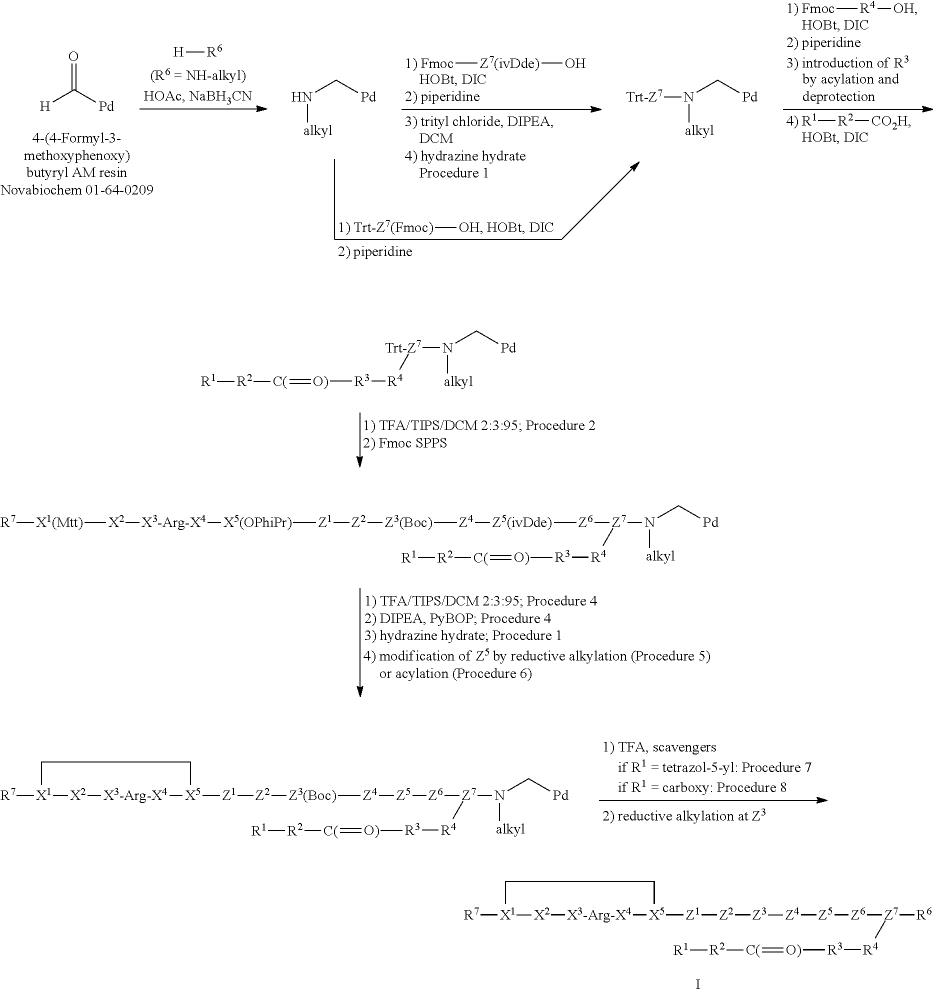

Possible Reaction Scheme for the Synthesis of Compounds According to Formula I with Disulfide Macrocycle, Z³ Containing an Alkylated Amino Group and R6=NH-alkyl

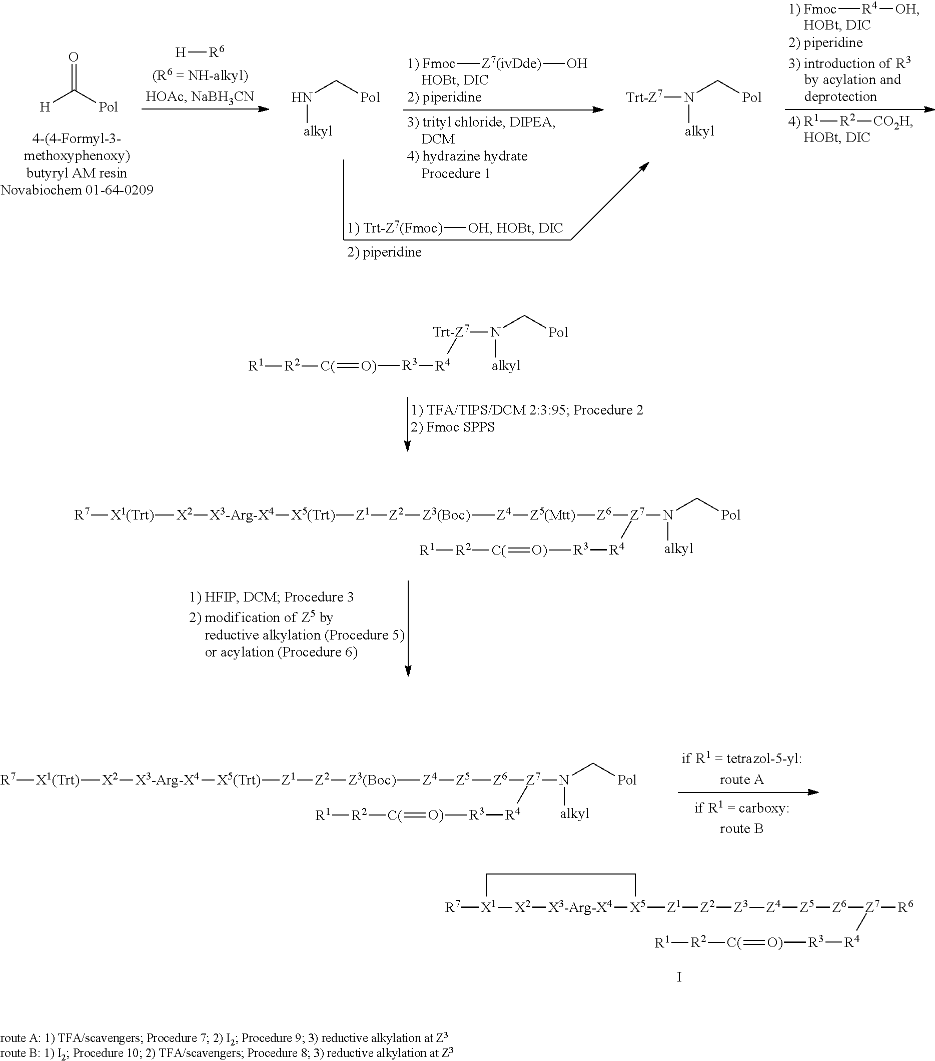

Possible Reaction Scheme for the Synthesis of Compounds According to Formula I with Lactam Macrocycle, Z³ Containing an Alkylated Amino Group and R⁶=dialkylamino

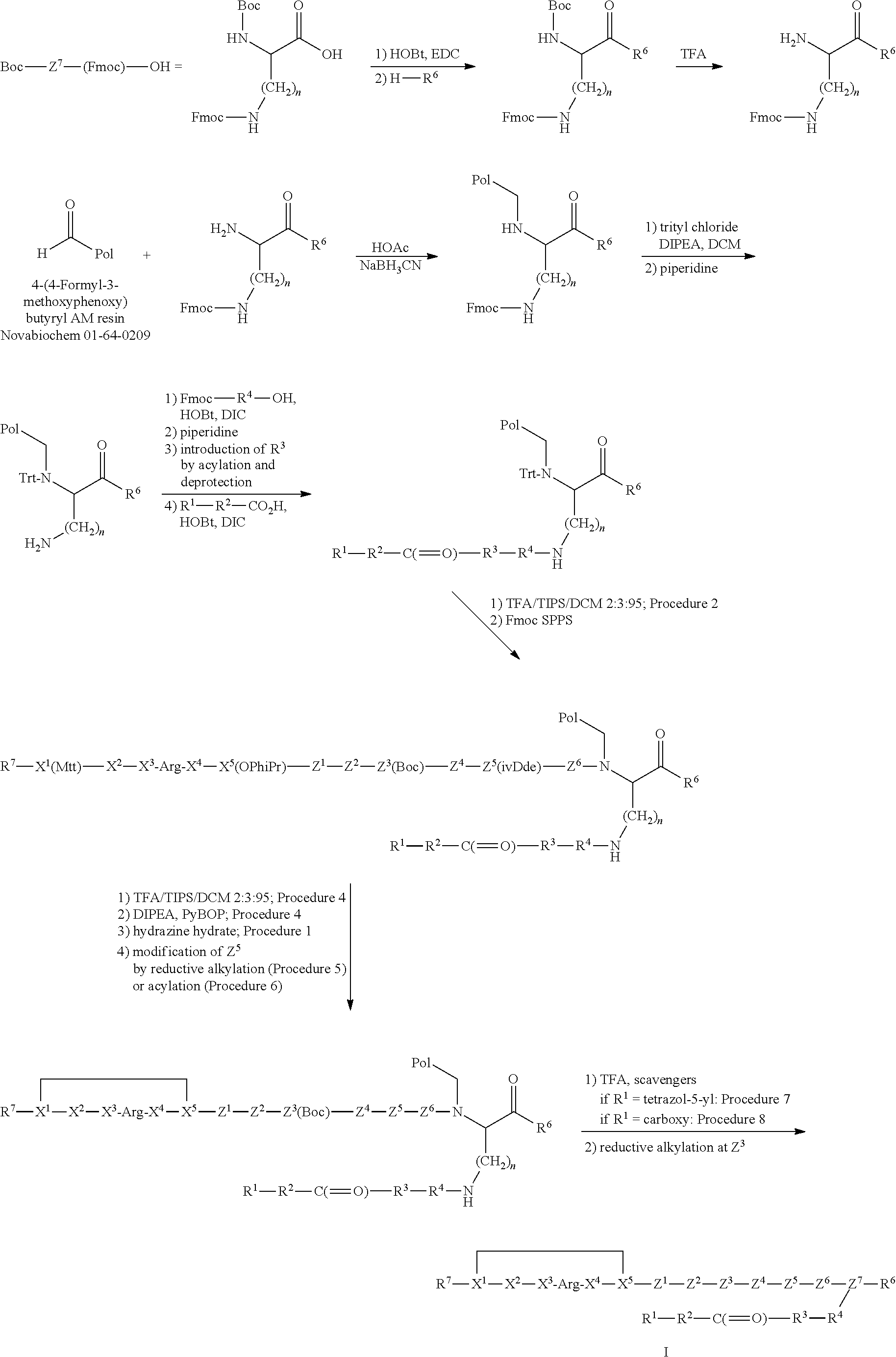

Possible Reaction Scheme for the Synthesis of Compounds According to Formula I with Disulfide Macrocycle, Z³ Containing an Alkylated Amino Group and R⁶=dialkylamino

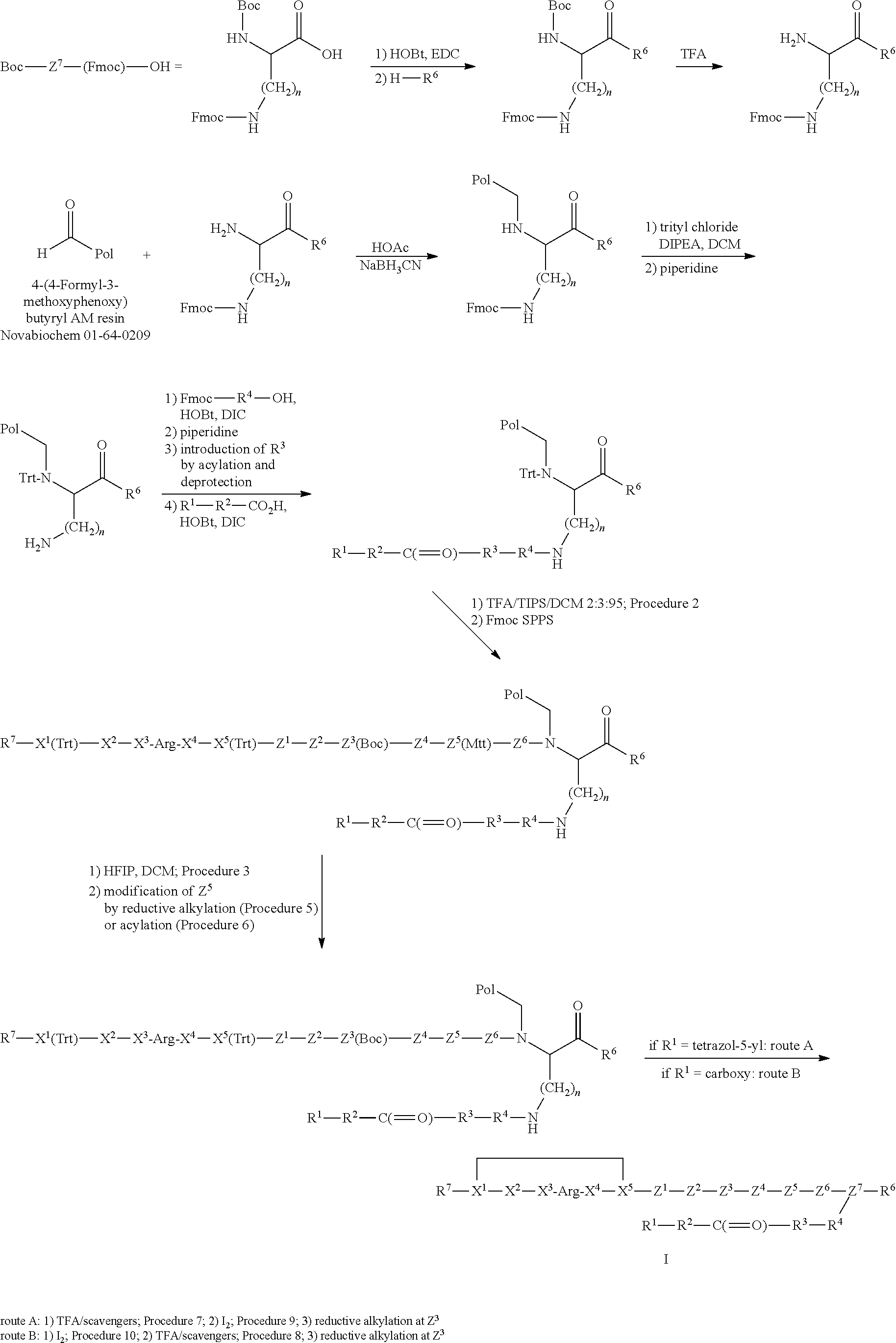

Possible Reaction Scheme for the Synthesis of Compounds According to Formula II with Lactam Macrocycle and Z³ Containing an Alkylated Amino Group

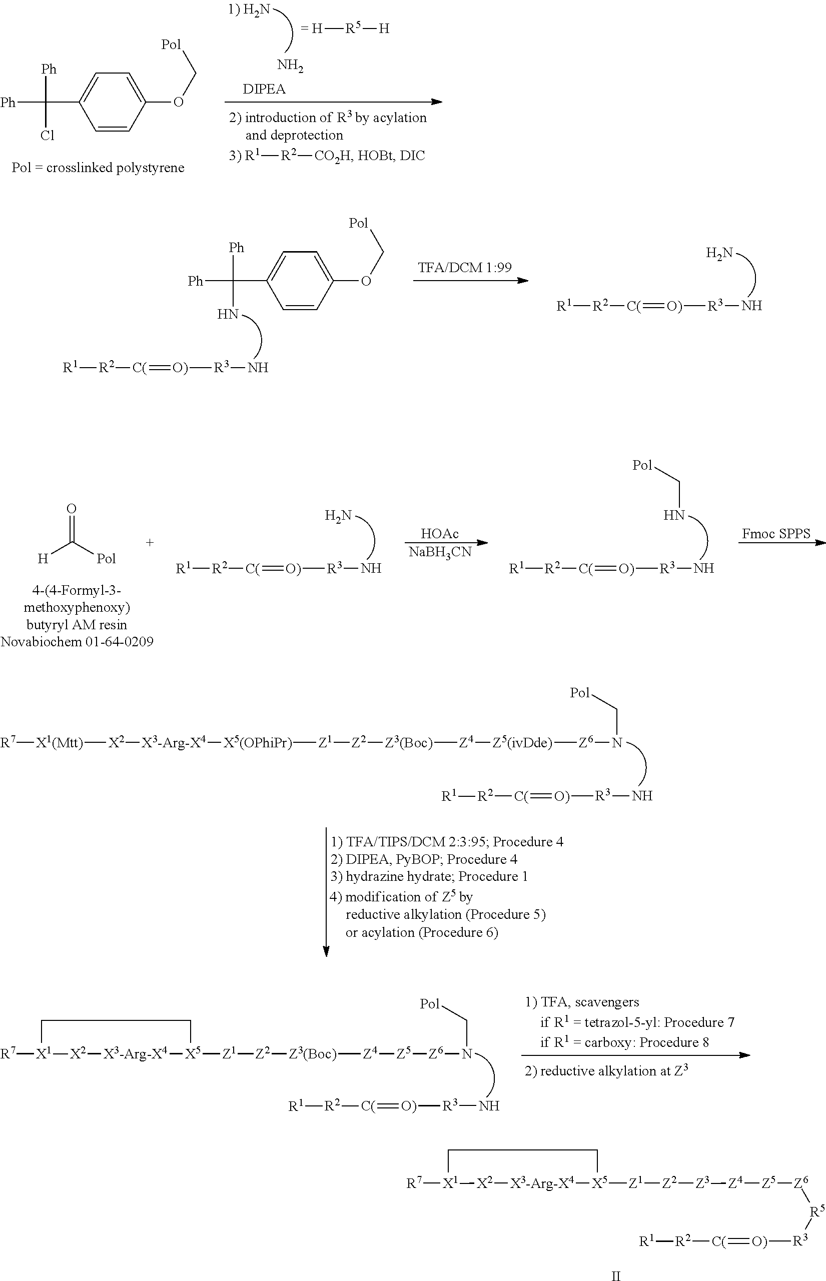

Possible Reaction Scheme for the Synthesis of Compounds According to Formula II with Disulfide Macrocycle and Z³ Containing an Alkylated Amino Group

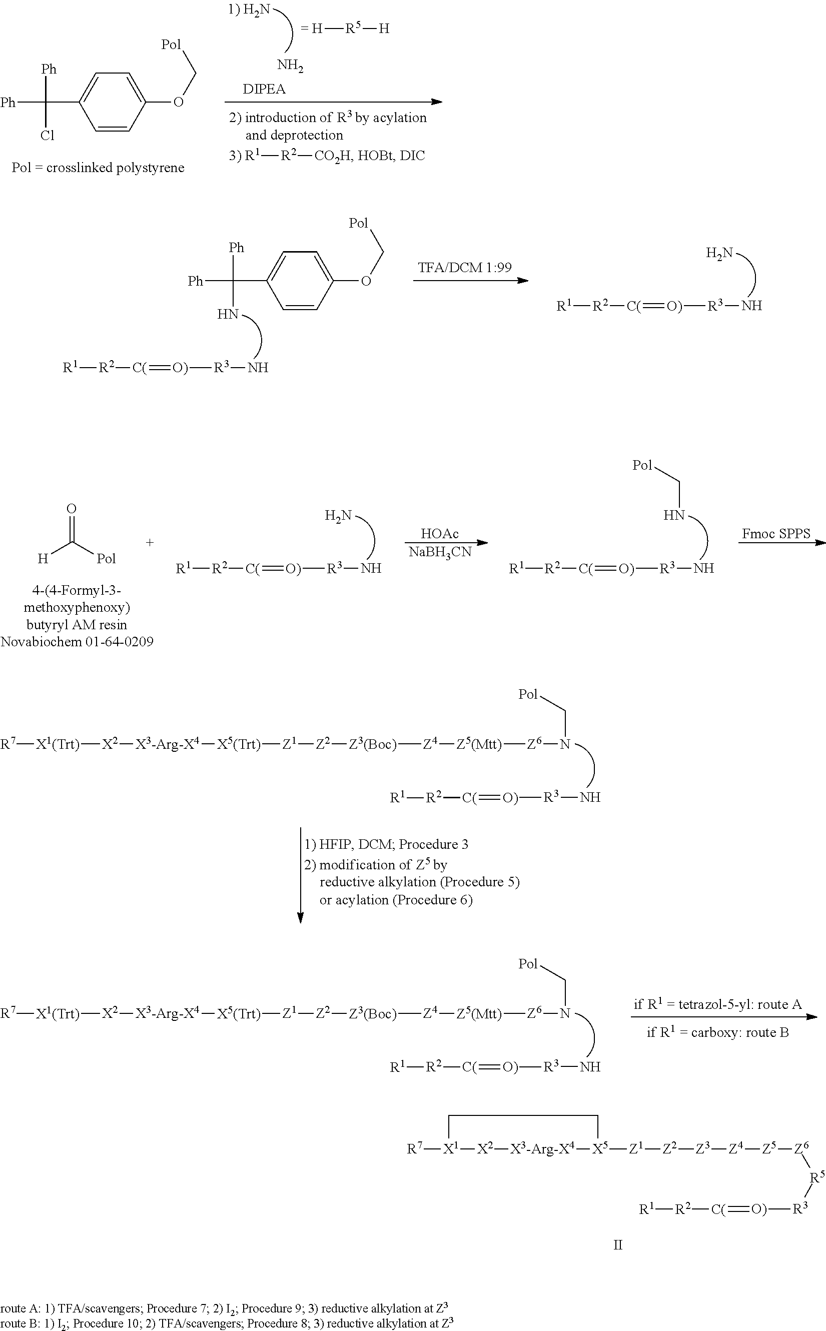

In the examples listed below, Rt values are retention times and the mass values are those detected by the mass spectroscopy (MS) detector and obtained using one of the following HPLC-MS or HPLC-MS devices, or MALDI-MS (matrix-assisted laser desorption ionization time of flight mass spectroscopy).

### LCMS (System 1a)

Waters Micromass LCT Premier XE mass spectrometer; electrospray; m/z=100 to m/z=2000; step 0.1 amu; Waters Acquity HPLC BEH C₁₈, 1.7 μm, 2.1 mm×50 mm; water/acetonitrile containing 0.1% formic acid; gradient 5% 95% acetonitrile linear during 4.0 min; flow 0.4 ml/min.

### LCMS (System 1b)

As described for system 1a, but with m/z=500 to m/z=2000.

### LCMS (System 1c)

As described for system 1a, but with m/z=700 to m/z=2500.

### LCMS (System 2)

Waters Micromass LCT Premier XE mass spectrometer; electrospray; m/z=500 to m/z=2000; step 0.1 amu; Waters Acquity HPLC BEH C₁₈, 1.7 μm, 2.1 mm×50 mm; water/acetonitrile containing 0.1% formic acid; gradient 5% 95% acetonitrile linear during 8.0 min; flow 0.4 ml/min.

### LCMS (System 3)

Sciex API-3000 Quadrupole MS, electrospray, m/z=300 to m/z=2000; column: Waters XTerra® MS C₁₈ 5 μm 3.0×50 mm; water/acetonitrile containing 0.05% TFA; gradient: 5%→90% acetonitrile from 0 to 7.5 min; flow 1.5 ml/min.

MALDI-MS

Molecular weights of the peptides were determined using matrix-assisted laser desorption ionization time of flight mass spectroscopy (MALDI-MS), recorded on a Microflex (Bruker Daltonics). A matrix of α-cyano-4-hydroxycinnamic acid was used.

Typical examples of synthesis procedures are as follows:

### Example 1

### Ac-c[Dap-His-D-Phe-Arg-Trp-Asp]-Pro-Pro-Arg-Asp-Lys(BCMA)-Lys[(2-{2-[16-(tetrazol-5-yl)hexadecanoylamino]ethoxy}ethoxy)acetyl]-NH₂

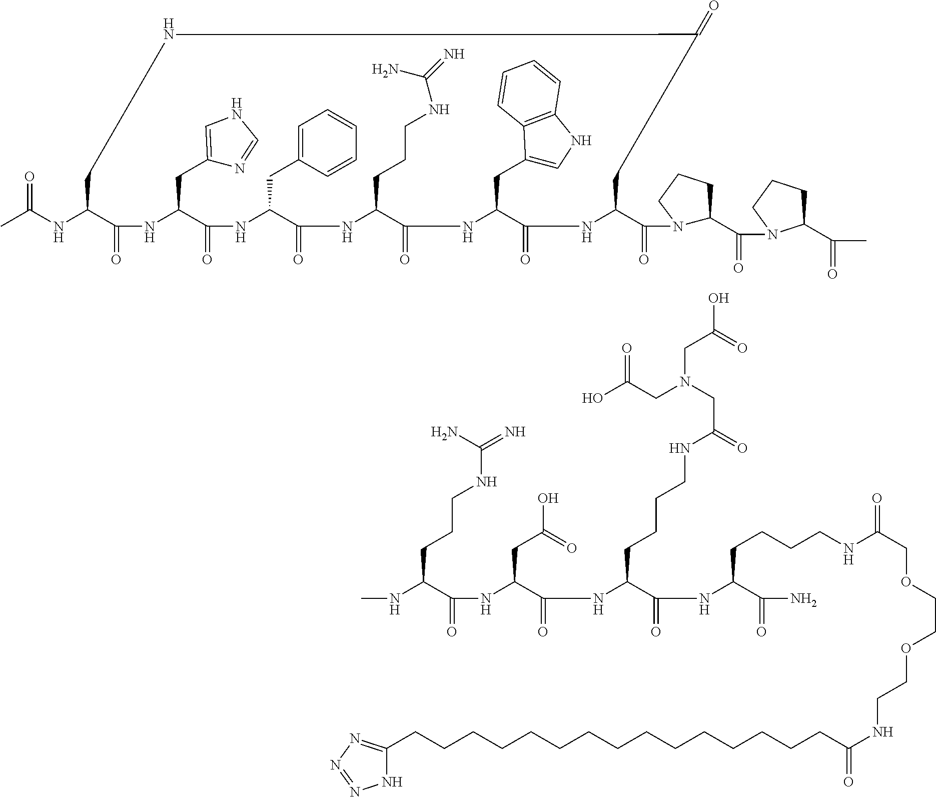

The synthesis was started using an Applied Biosystems 433 peptide synthesizer and Fmoc-Rink amide AM resin (4-(2′,4′-dimethoxyphenyl-Fmoc-aminomethyl)-phenoxyacetamidonorleucylaminomethylpolystyrene resin; 200-400 mesh; Novabiochem 01-64-0038) as starting material. Trt-Lys(Fmoc)-OH (N^{α}-trityl-N^{ε}-Fmoc-lysine; from Iris Biotech GmbH: FAA6570) was coupled to the deprotected resin. Fmoc was removed with piperidine in NMP. Fmoc-8-amino-3,6-dioxaoctanoic acid was then coupled to the side-chain nitrogen atom of Lys. Fmoc was removed. 16-(Tetrazol-5-yl)hexadecanoic acid (available by the synthetic procedure described in WO 2007/009894) was coupled to the resin to give Trt-Lys[(2-{2-[16-(tetrazol-5-yl)hexadecanoylamino]ethoxy}ethoxy)acetyl]-NH-RinkAM-polystyrene.

In a glass reactor with frit, the resin (0.25 mmol) was manually washed with a solution of 2.5% TIPS in DCM (40 ml). The resin was shaken with a solution of 2% TFA and 2.5% TIPS in DCM (40 ml) for 3 min and drained. This step was repeated another 7 times. The resin was washed with DCM (3×35 ml). It was shaken with 5% DIPEA in DCM (50 ml) for 10 min. The resin was then washed repeatedly with DCM, and was filled into a ABI reactor.

From this resin, the following resin-bound peptide chain was then assembled by automated synthesis on an ABI 433 peptide synthesizer: Fmoc-Dap(Mtt)-His(Trt)-D-Phe-Arg(Pmc)-Trp(Boc)-Asp(2-phenylisopropyloxy)-Pro-Pro-Arg(Pmc)-Asp(OtBu)-Lys(Boc)-Lys[(2-{2-[16-(tetrazol-5-yl)hexadecanoylamino]ethoxy}ethoxy)acetyl]-NH-RinkAM-polystyrene. The following steps were performed manually.

The resin was filled into a glass reactor with frit and washed extensively with DCM and finally once with 2.5% TIPS in DCM (50 ml). For selective side-chain deprotection of Dap(Mtt) and Asp(2-phenylisopropyloxy), the resin was shaken with a solution of 2% TFA and 2.5% TIPS in DCM (50 ml) for 10 min and drained. The resin was then washed with 2% TFA and 2.5% TIPS in DCM (50 ml) 8 times for 5 min. The resin was washed with DCM (3×30 ml), 5% DIPEA in DMF (25) and DMF (3×30 ml). For lactam cyclization, a solution of PyBOP (551 mg, 1.058 mmol), HOBt (143 mg, 1.058 mmol) and DIPEA (0.368 ml, 2.15 mmol) in DMF (30 ml) was added to the resin. The mixture was shaken overnight. The resin was washed several times with DCM.

Fmoc was removed by washing twice with 20% piperidine in NMP (1×2 min; 1×18 min). The resin was washed with NMP (6×30 ml). Acetylation was performed with a solution of acetic anhydride (0.185 ml, 2 mmol) in NMP (25 ml) for 1 h.

After several washings with DCM, the product was cleaved from the resin and purified similarly as described under general procedures to give 0.032 mmol (13% yield) of peptide Ac-c[Dap-His-D-Phe-Arg-Trp-Asp]-Pro-Pro-Arg-Asp-Lys-Lys[(2-{2-[16-(tetrazol-5-yl)hexadecanoylamino]ethoxy}ethoxy)acetyl]-NH₂ trifluoroacetate as a white solid.

Acylation at the Lys side chain can be performed in the following manner. In a small test tube, bis(tert-butoxycarbonylmethyl)aminoacetic acid (24 mg, 0.080 mmol; available by the synthetic procedure described above) and TSTU (24 mg, 0.080 mmol) are mixed with NMP (0.75 ml). DIPEA (0.033 ml, 0.192 mmol) is added. The tube is capped and shaken for 2 h. The resulting OSu ester solution is then used for the following acylation. In a test tube, the peptide TFA salt (0.032 mmol) is dissolved in NMP (1.5 ml). DIPEA (0.036 ml, 0.210 mmol) is added. To the resulting solution, the OSu ester solution is added. The tube is capped and shaken for 3 h. The reaction mixture is dropped into diethylether (40 ml). The resulting precipitate is collected by centrifugation and washed again with diethylether (40 ml). The liquid phase is removed by centrifugation. To the resulting solid or sticky residue, a premixed solution of triisopropylsilane (0.5 ml) and ethandithiol (0.5 ml) in TFA (9 ml) is added. The resulting solution is stirred for 90 min and then concentrated to give a liquid residue (appr. 2 ml). The liquid is treated with diethylether (40 ml) to give a white precipitate. The precipitate is collected by centrifugation, washed again with diethylether (40 ml) and dried to give a white solid.

Preparative HPLC (23-43% MeCN in water over 40 min) and freeze-drying afforded the peptide trifluoroacetate as a white solid.

LCMS (system 1a): Rt=1.87 min; ((M+2)/2)=1108.5

### Example 2

### Ac-c[Asp-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

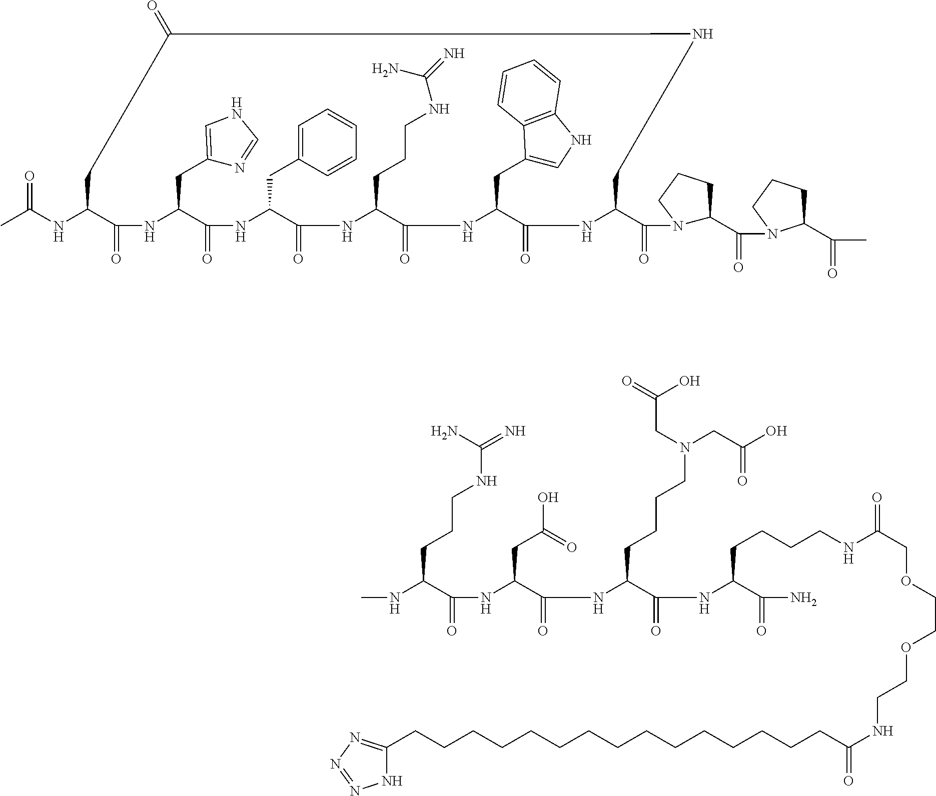 [SMILES: CC(=O)N[C@H]1CC(=O)NC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The synthesis was started from Fmoc-Rink amide AM resin (Novabiochem 01-64-0038).

### Removal of Fmoc:

The resin was shaken with 20% piperidine in NMP for 10 min. The liquid was filtered off. The resin was shaken again with 20% piperidine in NMP for 10 min and then washed six times with NMP.

Acylation with Fmoc-Lys(Mtt)-OH:

Fmoc-Lys(Mtt)-OH (2.4 eq) and HOBt (2.4 eq) were dissolved in NMP/DCM 1:1. DIC (2.4 eq) was added. The vial was capped, shaken and left to stand for 25 min. Then, the solution was added to the resin. The resin suspension was shaken. After 20 min, DIPEA (1.2 eq) was added and shaking was continued overnight. The resin was washed 4 times with NMP and 10 times with DCM.

Removal of Mtt from the Lys Side Chain:

The resin was shaken with a solution of 2% TFA and 2.5% TIPS in DCM for 5 min and drained. This procedure was repeated another 9 times (yellow filtrate from 2nd to 5th wash). The resin was washed 10 times with DCM, once with 5% DIPEA in NMP and 3 times with NMP.

### Acylation at Lys Side Chain:

{2-[2-(16-Tetrazol-5-yl)hexadecanoylamino)ethoxy]ethoxy}acetic acid (2.4 eq; available by the synthetic procedure described above) and HOBt (2.4 eq) were dissolved in NMP/DCM 1:1. DIC (2.4 eq) was added. The vial was capped, shaken and left to stand for 15 min. Then, the solution was added to the resin. The resin suspension was shaken overnight. The resin was washed 4 times with NMP.

### Peptide Synthesis:

From this resin, peptide resin Ac-Asp(2-phenylisopropyloxy)-His(Trt)-D-Phe-Arg(Pbf)-Trp(Boc)-Orn(Mtt)-Pro-Pro-Arg(Pbf)-Asp(OtBu)-Lys(Boc)-Lys[(2-{2-[16-(tetrazol-5-yl)hexadecanoylamino]ethoxy}ethoxy)acetyl]-NH-RinkAM-polystyrene was prepared using the corresponding Fmoc-amino acids. Fmoc removal was performed with 20% piperidine in NMP (2×10 min), followed by washing with NMP (6 times). Acylations were performed with Fmoc-amino acid (2.4 eq), HOBt (2.4 eq) and DIC (2.4 eq) in NMP/DCM 1:1 after preactivation for 10-20 min; acylation times from 2 h to overnight. Finally, acetyl was introduced by using acetic acid (4.8 eq), HOBt (4.8 eq) and DIC (4.8 eq) in NMP/DCM 1:1 after preactivation; acylation time overnight; then washing 3 times with NMP and 10 times with DCM.

### Selective Side-Chain Deprotection and Lactam Cyclization:

The resin was shaken with a solution of 2% TFA and 2.5% TIPS in DCM for 5 min and drained. This procedure was repeated another 9 times (yellow filtrate from 4th to 7th wash). The resin was washed 10 times with DCM, once with 5% DIPEA in NMP and 3 times with NMP. A solution of PyBOP (3 eq) and HOBt (3 eq) in NMP was added to the resin, followed by addition of DIPEA (6 eq). The mixture was shaken overnight. The resin was washed 3 times with NMP and 2 times with DCM.

Cleavage from the Resin and Side-Chain Deprotection:

The resin was washed 8 times with DCM. Then, it was shaken with a premixed solution (40 ml per mmol starting resin) of TFA/TIPS/mercaptoethanol 90:5:5 for 2 h. The liquid was then collected by filtration. The resin was washed with TFA-DCM and the filtrate was collected. The combined collected filtrate was treated with excess diethylether. The precipitated peptide was collected by centrifugation and washed another 2 times with diethylether to give a white solid.

### Reductive Alkylation at Lys Side Chain:

The crude peptide (from 0.125 mmol of Rink AM resin) was dissolved in a mixture of MeOH (4.25 ml), N-methylformamide (2.5 ml) and 0.2 M citrate buffer pH 4.5 (4.5 ml, 0.9 mmol; preparation of the buffer: 0.2 mol of citric acid and 0.35 mol of NaOH dissolved in one liter of water). Glyoxalic acid monohydrate (0.212 g, 2.3 mmol) and a freshly prepared solution of sodium cyanoborohydride (0.082 g, 1.3 mmol) in MeOH (1.5 ml) were added. The mixture was stirred for 4 days (not necessary; 24 h would have been fine). LCMS indicated completed N,N-dialkylation.

### Purification:

The mixture was acidified with TFA (0.25 ml) and diluted with water to give a total volume of 32 ml. HPLC purification (0.1% TFA; 20-40% MeCN in water over 40 min) and freeze-drying afforded the peptide trifluoroacetate as a white solid. Based on a nitrogen-specific HPLC detector (see above), the obtained yield of product was corresponding to 54 mg of the TFA-free peptide (20% yield from 0.125 mmol of Rink AM resin).

LCMS (system 1b): Rt=1.87 min; ((M+2)₁₋₂)=1094.0

### Example 3

### Ac-c[Dab-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl)

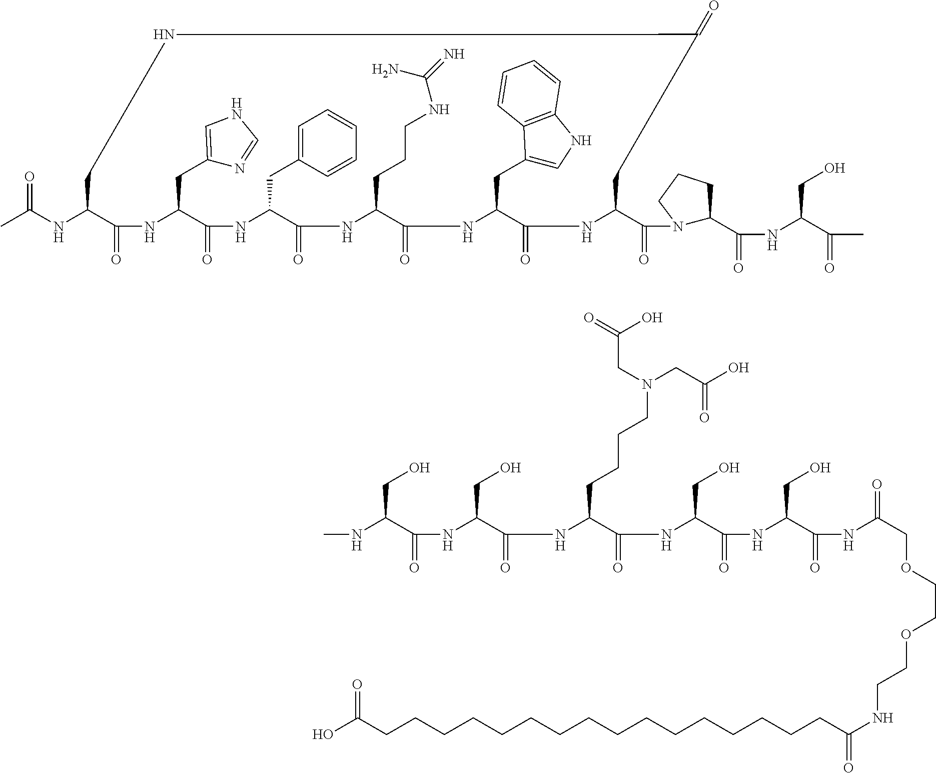

Attachment of Octadecanedioic acid to Bromo-Wang Resin:

In a glass reactor with frit, bromo-Wang polystyrene resin (1.429 g, 1.5 mmol; (4-bromomethyl)phenoxymethyl polystyrene, 1.05 mmol/g, Novabiochem 01-64-0186) was washed with DCM (15 ml). A solution prepared from octadecanedioic acid (3.3019 g, 10.5 mmol), DCM (5 ml), NMP (10 ml) and DIPEA (1.978 ml, 11.55 mmol) was added to the resin. The reactor was stoppered and the mixture was shaken for 23 h. The liquid was filtered off and the resin was washed with NMP (7×15 ml) and DCM (15 ml).

Amide Formation with Diamine:

NMP (5 ml), DCM (2.5 ml), DIPEA (1.027 ml, 6.0 mmol) and 1,10-diaza-4,7-dioxadecane (2.197 ml, 15.0 mmol) were added to the resin. The resin was shaken. A solution of PyBOP (3.122 g, 6.0 mmol) in a mixture of NMP (5 ml) and DCM (2.5 ml) was added. The reactor was stoppered and the mixture was shaken for 2½ h. The liquid was filtered off and the resin was washed with NMP (4×15 ml). The resin in the glass reactor was then washed twice for 15 min with 20% water in NMP (2×15 ml) (in order to hydrolyse phosphonium salts). Washing was continued with NMP (15 ml), THF (4×15 ml), DCM (15 ml) and NMP (15 ml).

Acylation with Fmoc-Ser(tBu)-OH:

In a separate glass vial, the carboxylic acid (1.726 g, 4.5 mmol) was dissolved in 1M HOBt-NMP solution (4.5 ml, 4.5 mmol), NMP (3 ml) and DCM (7.5 ml). DIC (0.736 ml, 4.725 mmol) was quickly added and the mixture was shaken immediately thereafter. The vial was capped and the mixture was shaken for 40 min. The resulting yellowish solution was added to the resin. The glass reactor was stoppered and shaken for 4 h. The liquid was filtered off and the resin was washed with NMP (3×15 ml), THF (3×15 ml) and DCM (4×15 ml). The resin was dried for 65 h in vacuum to give a yellowish resin (1.849 g).

### Estimation of the Resin Loading:

1.849 g−1.429 g=0.420 g attached to the resin. Molecular weight of the target product attached to the resin minus molecular weight of bromide=809.09 g/mol−79.90 g/mol=729.19 g/mol.

0.420 g/729.19 g/mol=0.576 mmol from 1.5 mmol starting resin.

This means the maximum yield of product resin would have been 0.576 mmol, but was probably less if impurities were attached to the resin. Maximum loading would have been 0.576 mmol/1.849 g=0.31 mmol/g, but was probably less.

### Peptide Synthesis:

From resin Fmoc-Ser(tBu)-NH—(CH₂)₂—O—(CH₂)₂—O—(CH₂)₂—NH—CO—(CH₂)₁₆—C0-O-Wang-polystyrene (0.924 g, 0.286 mmol if no impurities attached to the resin; originating from 0.75 mmol of bromo-Wang polystyrene), peptide resin Fmoc-His(Trt)-D-Phe-Arg(Pbf)-Trp(Boc)-Asp(2-phenylisopropyloxy)-Pro-Ser(tBu)-Ser(tBu)-Ser(tBu)-Lys(Boc)-Ser(tBu)-Ser(tBu)-NH—(CH₂)₂—O—(CH₂)₂—O—(CH₂)₂—NH—CO—(CH₂)₁₆—CO—O-Wang-polystyrene was prepared on a CEM Liberty microwave peptide synthesizer using Fmoc-amino acids (HOAt/DIC-mediated couplings without base and without preactivation; Fmoc removal with piperidine in NMP).

### Attachment of Fmoc-Dab(Mtt)-OH:

In a separate glass vial, Fmoc-Dab(Mtt)-OH (0.746 g, 1.25 mmol) was dissolved in 1M HOBt-NMP solution (1.25 ml, 1.25 mmol), NMP (2.25 ml) and DCM (0.5 ml). This solution was added to the resin, followed by addition of PyBOP (0.650 g, 1.25 mmol) and DIPEA (0.428 ml, 2.5 mmol). The mixture was shaken for 4 h and then filtered. The reaction was repeated with the same amount of reagents and solvents for 2 h. the resin was washed with NMP (5×10 ml) and DCM (5×10 ml).

Deprotection and Acylation with Acetic Acid:

The resin was shaken with 20% piperidine in NMP (10 ml) for 5 min, drained, shaken with 20% piperidine in NMP (10 ml) for 20 min, drained and washed with NMP (7×10 ml). To the resin was added a solution prepared from acetic acid (0.071 ml, 1.25 mmol), 1M HOBt-NMP solution (1.25 ml, 1.25 mmol), NMP (0.75 ml) and DCM (2 ml). PyBOP (0.650 g, 1.25 mmol) and DIPEA (0.445 ml, 2.6 mmol) were added. The mixture was shaken for 1 h and then filtered. The resin was washed with NMP (5×10 ml) and DCM (5×10 ml).

### Selective Side-Chain Deprotection and Lactam Cyclization:

The resin was shaken with a solution of 2% TFA and 3% TIPS in DCM (10 ml) for 2 min and drained. This procedure was repeated another 9 times. The resin was washed with DCM (4×10 ml), 10% DIPEA in NMP (2×10 ml) and DCM (3×10 ml). A solution of PyBOP (0.468 g, 0.9 mmol) and HOAt (0.122 g, 0.9 mmol) in DCM (2 ml) and NMP (2 ml) was added to the resin, followed by addition of DIPEA (0.308 ml, 1.8 mmol). The mixture was shaken overnight. The resin was washed with NMP (5×10 ml) and DCM (6×10 ml).

Cleavage from the Resin and Side-Chain Deprotection:

The resin was shaken with a premixed solution of TFA/TIPS/water 95:2.5:2.5 (10 ml) for 2½ h. The liquid was then collected by filtration. The resin was washed with TFA/DCM 1:2 (15 ml) and the filtrate was collected. The combined collected filtrate was concentrated and then treated with diethylether (40 ml). The precipitated peptide was collected by centrifugation and washed another 2 times with diethylether to give a white solid.

### Reductive Alkylation at Lys Side Chain:

The crude peptide was dissolved in a mixture of N-methylformamide (5 ml), MeOH (8.5 ml) and 0.2 M citrate buffer pH 4.5 (4.5 ml, 0.9 mmol; preparation of the buffer: 0.2 mol of citric acid and 0.35 mol of NaOH dissolved in one liter of water). Glyoxalic acid monohydrate (0.212 g, 2.3 mmol) and a freshly prepared solution of sodium cyanoborohydride (0.057 g, 0.91 mmol) in MeOH (0.6 ml) were added. The mixture was stirred for 3 days. LCMS indicated incomplete reaction. Glyoxalic acid monohydrate (0.106 g, 1.15 mmol) dissolved in 0.2 M citrate buffer pH 4.5 (4.5 ml, 0.9 mmol) was added. A freshly prepared solution of sodium cyanoborohydride (0.107 g, 0.1.7 mmol) in MeOH (2 ml) was added. Some precipitate was observed. Addition of N-methylformamide (1 ml) and gentle warming resulted in complete dissolution. The mixture was stirred for 24 h. LCMS indicated completed N,N-dialkylation.

### Purification:

MeOH was evapourated off. The mixture was acidified with TFA (0.25 ml) and diluted with water to give a total volume of 20 ml. HPLC purification (0.1% TFA; 27-39% MeCN in water over 40 min) and freeze-drying afforded the peptide trifluoroacetate as a white solid. Based on a nitrogen-specific HPLC detector (see above), the obtained yield of product was corresponding to 38 mg of the TFA-free peptide (2% yield from 0.75 mmol of bromo-Wang resin).

LCMS (system 3): Rt=3.96 min; ((M+2)/2)=1045.1

### Example 4

### Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-OH

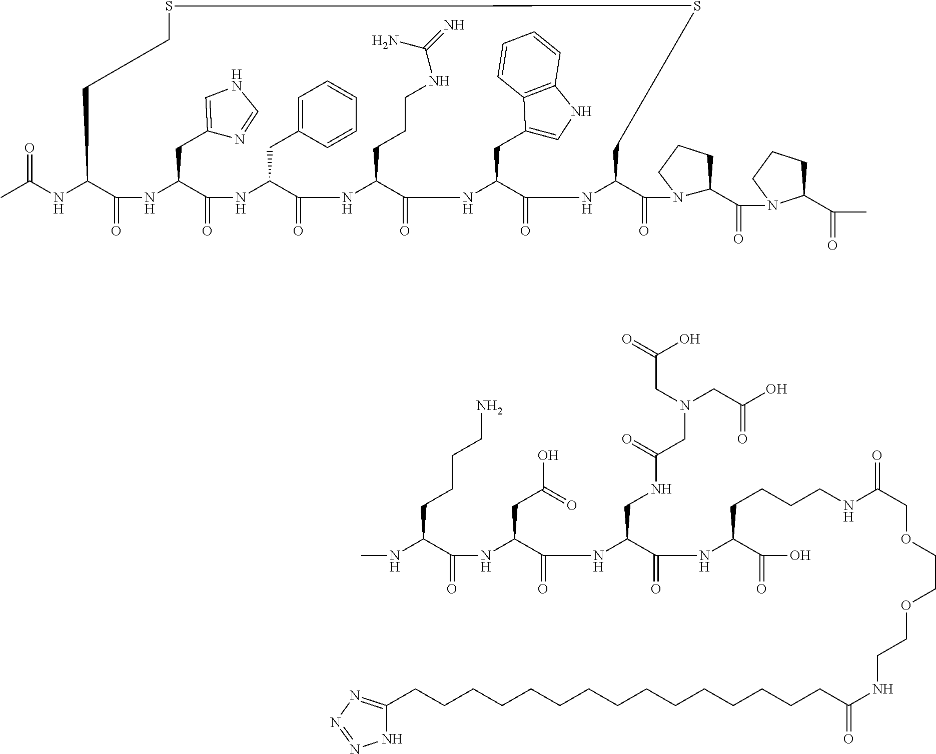 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(=O)O]

### Attachment of Fmoc-Lys(ivDde) to Wang Resin:

In a glass reactor with frit, Wang polystyrene resin (0.216 g, 0.25 mmol; p-benzyloxybenzyl alcohol resin; crosslinked polystyrene; 1.16 mmol/g) was washed with NMP and drained. In a separate flask with magnetic stirrer, Fmoc-Lys(ivDde)-OH (1.44 g, 2.50 mmol) was dissolved in a mixture of DCM (10 ml) and DMF (1 ml). A freshly prepared solution of DIC (0.194 ml, 1.25 mmol) in DCM (1 ml) was added to the and the mixture was stirred in a capped flask. After about 15 min, a precipitate was observed. DMF was added until all was dissolved again. Stirring was continued. After about 1 h, this solution was added to the resin. A solution of DMAP (3 mg, 0.025 mmol) in DMF (0.5 ml) was added to the resin. The reactor was stoppered and shaken for 2 h. The resin was washed 5 times with NMP and 5 times with DCM.

### Peptide Synthesis:

From the resulting resin Fmoc-Lys(ivDde)-O-Wang-polystyrene, peptide resin Ac-homoCys(Trt)-His(Trt)-D-Phe-Arg(Pbf)-Trp(Boc)-Cys(Trt)-Pro-Pro-Lys(Boc)-Asp(OtBu)-Dap(Mtt)-Lys(ivDde)-O-Wang-polystyrene was prepared on a CEM Liberty microwave peptide synthesizer using Fmoc-amino acids (HOAt/DIC-mediated couplings without base and without preactivation; Fmoc removal with piperidine in NMP; acetylation with 1M acetic anhydride in NMP).

### Removal of Mtt:

The resin was washed 5 times with DCM and then 2 times for 15 min with 15 ml of HFIP/DCM/TIPS 70:28:3. The resin was washed 5 times with DCM, 5 times with NMP, 3 times with 10% DIPEA in NMP and 5 times with NMP.

### Acylation:

A solution prepared from bis(tert-butoxycarbonylmethyl)aminoacetic acid (182 mg, 0.6 mmol), NMP (10 ml), PyBOP (313 mg, 0.6 mmol) and 2,4,6-collidine (0.166 ml, 1.25 mmol) was added to the resin. The mixture was shaken overnight. The resin was washed 5 times with NMP.

Removal of ivDde:

The resin was washed 3 times for 20 min with 3% hydrazine hydrate in DMF. The resin was washed with DMF, 5% HOBt in NMP, 5 times with NMP and 5 times with DCM.

Acylation with Fmoc-8-amino-3,6-dioxaoctanoic acid:

In a separate vial, the carboxylic acid (386 mg, 1.0 mmol) and HOAt (137 mg, 1.0 mmol) were dissolved in NMP (10 ml). DIC (0.155 ml, 1.0 mmol) was added and the mixture was shaken immediately thereafter. The vial was capped and the mixture was stirred for 30 min. The resulting solution was added to the resin, followed by addition of 2,4,6-collidine (0.133 ml, 1.0 mmol). The mixture was shaken overnight. The liquid was filtered off and the resin was washed 5 times with NMP.

### Removal of Fmoc:

The resin was washed twice for 20 min with 20% piperidine in NMP. The resin was washed 6 times with NMP.

Acylation with 16-(tetrazol-5-yl)hexadecanoic acid:

In a separate vial, the carboxylic acid (325 mg, 1.0 mmol) and HOAt (137 mg, 1.0 mmol) were dissolved in NMP (6 ml) and DCM (8 ml). DIC (0.155 ml, 1.0 mmol) was added and the mixture was shaken immediately thereafter. The vial was capped and the mixture was stirred for 15 min. The resulting solution was added to the resin, followed by addition of 2,4,6-collidine (0.133 ml, 1.0 mmol). The mixture was shaken overnight. The liquid was filtered off and the resin was washed 5 times with NMP and 5 times with DCM.

Cleavage from the Resin and Side-Chain Deprotection:

The resin was washed with DCM/TIPS/mercaptoethanol 90:5:5, drained and then shaken for 3 h with a premixed solution of TFA/EDT/TIPS/water 90:5:4:1 (20 ml). The liquid was collected by filtration. The resin was washed with TFA/EDT/TIPS/water 90:5:4:1 (15 ml) and the filtrate was collected. The combined collected filtrate was concentrated under reduced pressure and thereafter treated with diethylether (30 ml). The precipitated peptide was collected by centrifugation and washed another 2 times with diethylether to give a white solid.

### Disulfide Cyclisation in Solution:

The crude peptide (from 0.25 mmol of Wang resin) was dissolved in MeOH/water 95:5 (75 ml). A solution of iodine (254 mg, 1.0 mmol) in DCM/MeOH 1:4 (5 ml) was added slowly over 5 min to the peptide solution with stirring. The mixture was stirred for 30 min and then quenched with a solution of ascorbic acid (177 mg, 1.0 mmol) in water. The mixture was concentrated under reduced pressure.

Preparative HPLC and freeze-drying afforded the peptide trifluoroacetate as a white solid. Based on a nitrogen-specific HPLC detector (see above), the obtained yield of product was corresponding to 7.1 mg of the TFA-free peptide (1% yield from 0.25 mmol of Wang resin).

LCMS (system 1b): Rt=1.92 min; ((M+2)/2)=1091.5

### Example 5

### Ac-c[homoCys-His-D-Phe-Arg-Trp-Cys]-Pro-Pro-Lys-Glu-Lys(biscarboxymethyl)-Lys({2-[2-(15-carboxypentadecanoylamino)ethoxy]ethoxy}acetyl)-NH₂

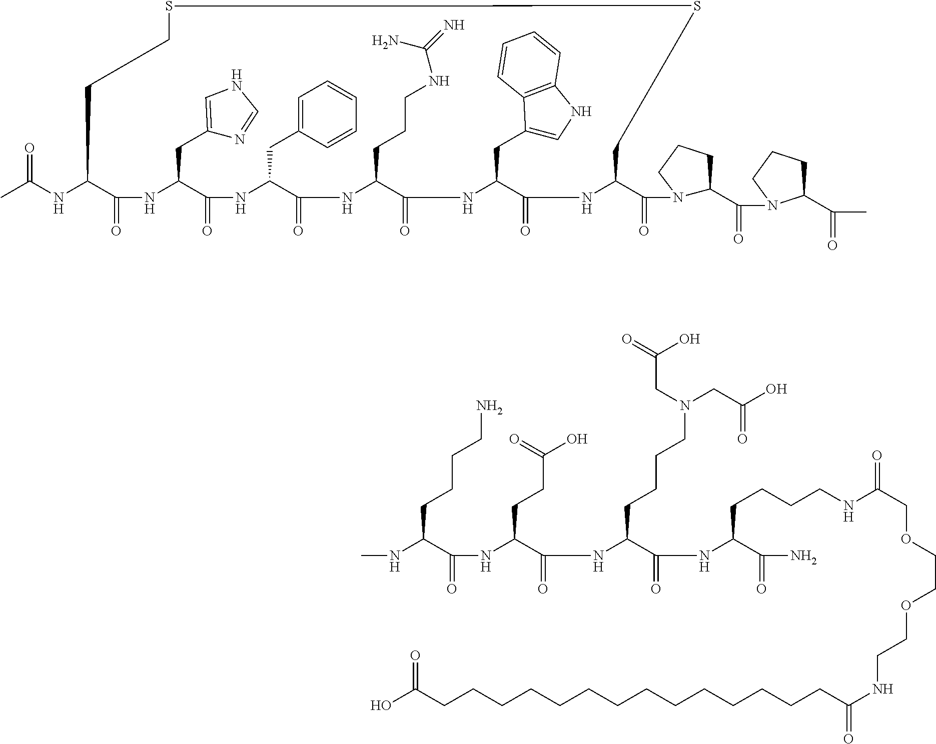

### Peptide Synthesis:

From Fmoc-Rink amide AM polystyrene resin (404 mg, 0.25 mmol; 0.62 mmolg), peptide resin Ac-homoCys(Trt)-His(Trt)-D-Phe-Arg(Pbf)-Trp(Boc)-Cys(Trt)-Pro-Pro-Lys(Boc)-Glu(OtBu)-Lys(ivDde)-Lys(Mtt)-NH-RinkAM-polystyrene was prepared on a CEM Liberty microwave peptide synthesizer using Fmoc-amino acids (HOAt/DIC-mediated couplings without base and without preactivation; Fmoc removal with piperidine in NMP; acetylation with 1M acetic anhydride in NMP; extensive DCM washing after completed synthesis).

### Removal of Mtt:

The resin was washed with DCM (2×10 ml) and then 2 times for 15 min with 15 ml of HFIP/DCM/TIPS 70:28:3. The resin was washed with DCM (2×10 ml), 10% DIPEA in NMP (2×10 ml) and NMP (4×10 ml).

Acylation with Fmoc-8-amino-3,6-dioxaoctanoic acid:

In a separate vial, the carboxylic acid (347 mg, 0.9 mmol), 1M HOBt-NMP solution (0.9 ml, 0.9 mmol), NMP (1.1 ml) and DCM (2.0 ml) were mixed to give a solution. DIC (0.140 ml, 0.9 mmol) was added and the mixture was shaken immediately thereafter. The vial was capped and the mixture was left to stand for 20 min. The resulting solution was added to the resin. The mixture was shaken for 90 min. The liquid was filtered off and the resin was washed with NMP/DCM 1:1 (5×10 ml).

### Removal of Fmoc:

The resin was washed shaken with 20% piperidine in NMP (10 ml) for 5 min, drained, and shaken with 20% piperidine in NMP (10 ml) for 15 min. The resin was washed with NMP (7×10 ml).

Acylation with hexadecanedioic acid mono-tert-butyl ester:

In a separate vial, the carboxylic acid (308 mg, 0.9 mmol; available by the synthetic procedure described in: U. Widmer, Synthesis 1983, 135) 1M HOBt-NMP solution (0.9 ml, 0.9 mmol), NMP (1.1 ml) and DCM (2.0 ml) were mixed to give a solution. DIC (0.140 ml, 0.9 mmol) was added and the mixture was shaken immediately thereafter. The vial was capped and the mixture was left to stand for 20 min. The resulting solution was added to the resin. The mixture was shaken for 18 h. The liquid was filtered off and the resin was washed with NMP/DCM 1:1 (5×10 ml).

Removal of ivDde:

The resin was washed 3 times for 30 min with 3% hydrazine hydrate in DMF. The resin was washed with NMP (10 ml), DCM (10 ml), 5% HOBt in NMP (10 ml), NMP (5×10 ml) and DCM (2×10 ml).

### Solid-Phase Reductive Alkylation:

A solution of glyoxalic acid monohydrate (258 mg, 2.8 mmol) in MeOH (1.8 ml) and NMP (6.6 ml) was added to the resin. HOAc (0.160 ml, 2.8 mmol) and a freshly prepared solution of sodium cyanoborohydride (201 mg, 3.2 mmol) in MeOH (1.8 ml) and NMP (6.6 ml) were added. The mixture was shaken overnight. The resin was washed with NMP (5×10 ml).

### Solid-Phase Disulfide Cyclisation:

The resin was shaken with a solution of iodine (632 mg, 2.5 mmol) in NMP (10 ml) for 1 h and then drained. The resin was washed with NMP (5×10 ml) and then shaken for 10 min with a solution of ascorbic acid (132 mg, 0.75 mmol) in water (0.75 ml) and NMP (10 ml). The resin was washed with NMP/MeOH 4:1 (4×10 ml), NMP (3×10 ml) and DCM (6×10 ml).

Cleavage from the Resin and Side-Chain Deprotection:

The resin was shaken for 2½ h with a premixed solution of TFA/TIPS/water 95:2.5:2.5 (10 ml). The liquid was collected by filtration. The resin was washed with DCM/TFA 2:1 (15 ml) and the filtrate was collected. The combined collected filtrate was concentrated under reduced pressure and thereafter treated with diethylether (45 ml). The precipitated peptide was collected by centrifugation and washed with diethylether (2×45 ml) to give a white solid.

HPLC purification (0.1% TFA; 25-37% MeCN in water over 40 min) and freeze-drying afforded the peptide trifluoroacetate as a white solid. Based on a nitrogen-specific HPLC detector (see above), the obtained yield of product was corresponding to 51 mg of the TFA-free peptide (10% yield from 0.25 mmol of Rink AM resin).

LCMS (system 1b): Rt=1.82 min; ((M+2)/2)=1071.5

### Example 6

### Ac-c[Glu-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

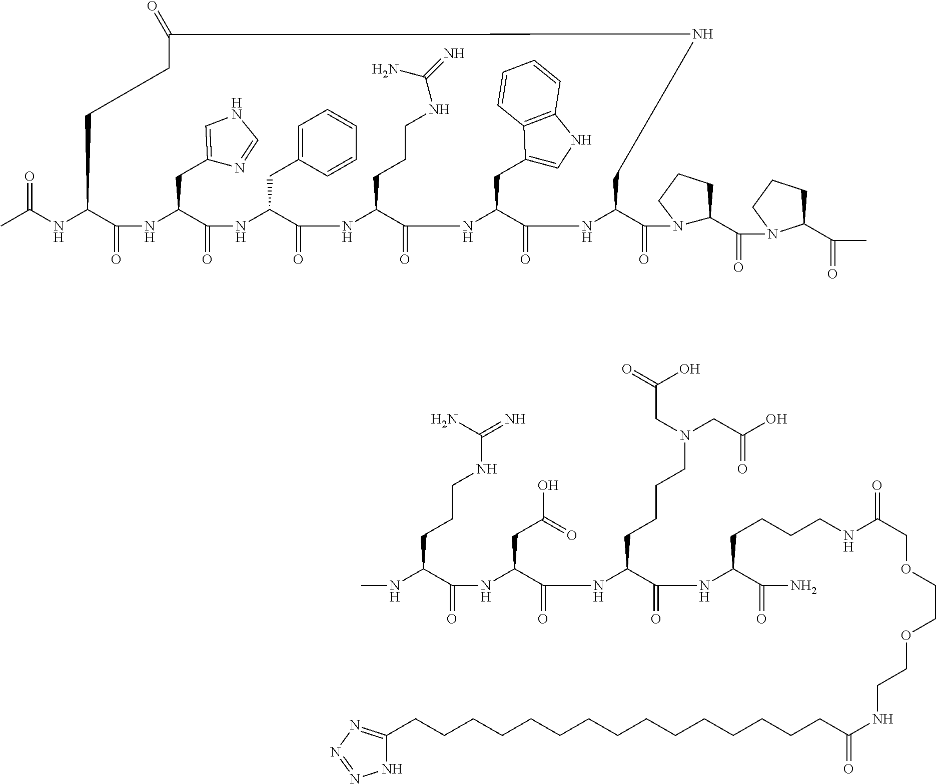 [SMILES: CC(=O)N[C@H]1CCC(=O)NC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Dap(Mtt)-Pro-Pro-Arg(Pbf)-Asp(OtBu)-Lys(Boc)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with 16-(tetrazol-5-yl)hexadecanoic acid+HOBt/DIC, cleavage from the resin and side-chain deprotection according to Procedure 7, reductive alkylation according to Procedure 11, HPLC purification and freeze-drying.

LCMS (system 1b): Rt=1.92 min; ((M+2)/2)=1087.0

### Example 7

### Ac-c[Lys-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂

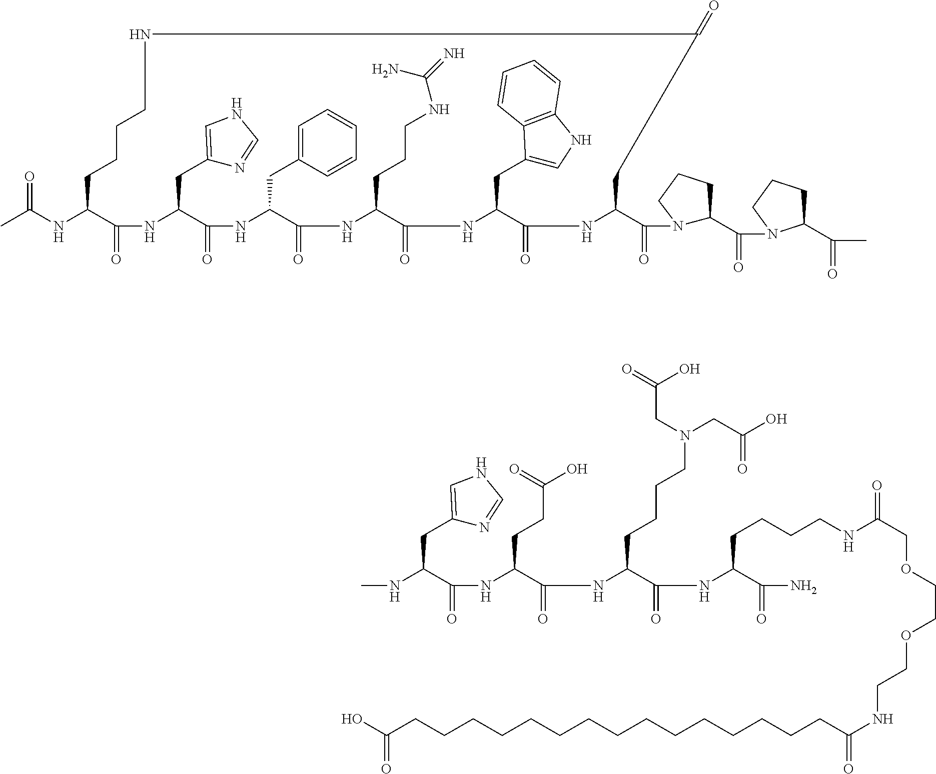 [SMILES: CC(=O)N[C@H]1CCCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]

The compound can be prepared by the following synthetic route: Fmoc-based synthesis of peptide resin Ac-Lys(Mtt)-His(Trt)-D-Phe-Arg(Pbf)-Trp(Boc)-Glu(2-phenylisopropyloxy)-Pro-Pro-His(Trt)-Glu(OtBu)-Lys(Boc)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with heptadecanedioic acid mono-tert-butyl ester (available by the synthetic procedure described above, or from heptadecanedioic acid by the procedure described in: U. Widmer, Synthesis 1983, 135)+HOBt/DIC, cleavage from the resin and side-chain deprotection according to Procedure 8, reductive alkylation according to Procedure 11, HPLC purification and freeze-drying.

LCMS (system 1b): Rt=2.08 min; ((M+2)/2)=1094

### Example 8

### Ac-c[Orn-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

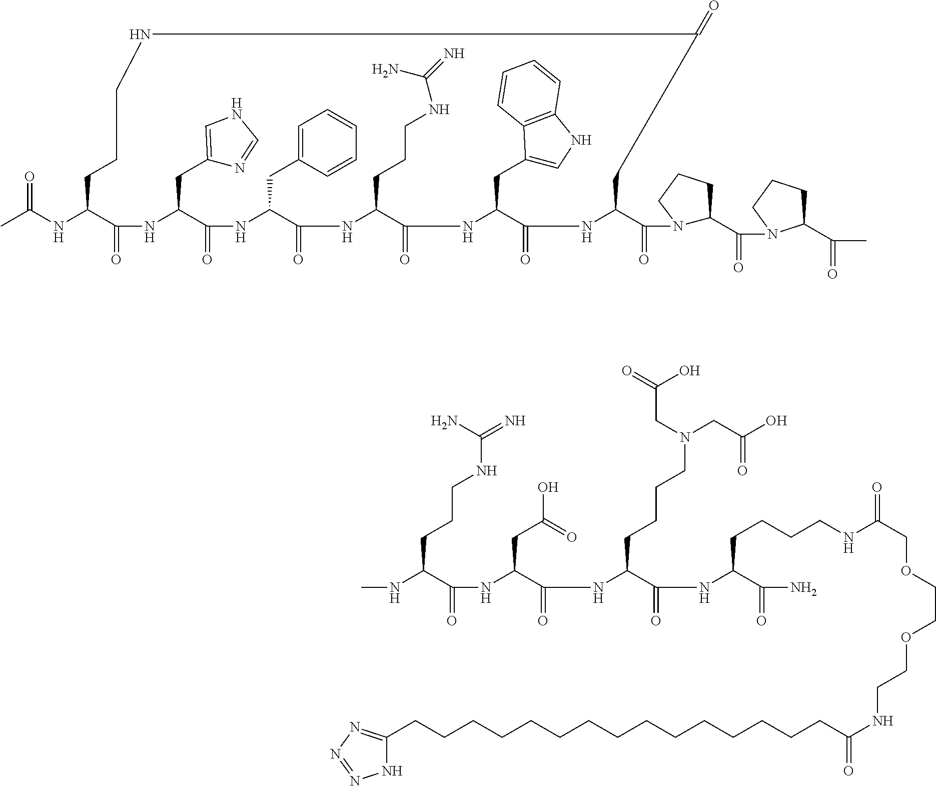 [SMILES: CC(=O)N[C@H]1CCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Orn(Mtt)-His(Trt)-D-Phe-Arg(Pbf)-Trp(Boc)-Glu(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 1b): Rt=2.06 min; ((M+2)/2)=1101.1

### Example 9

### Ac-c[Dab-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl)

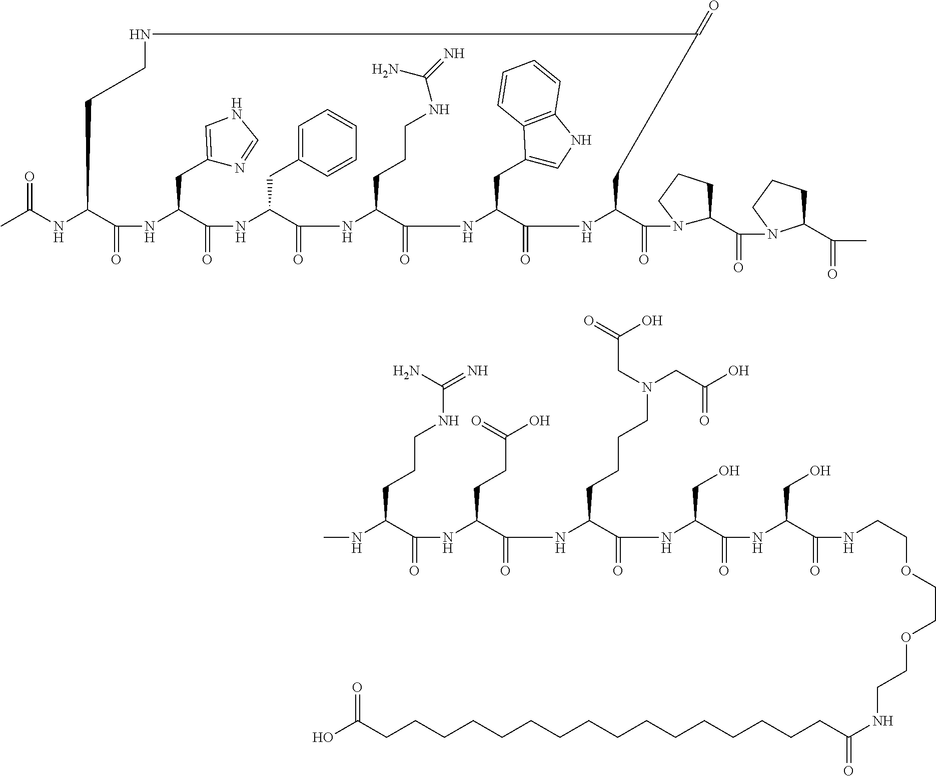

The compound can be prepared by the following synthetic route: preparation of resin Fmoc-Ser(tBu)-NH—(CH₂)₂—O—(CH₂)₂—O—(CH₂)₂—NH—CO—(CH₂)₁₆—CO—O-Wang-polystyrene as described for Example 3, then Fmoc-based synthesis of peptide resin Ac-Dab(Mtt)-His(Trt)-D-Phe-Arg(Pbf)-Trp(Boc)-Asp(2-phenylisopropyloxy)-Pro-Pro-Arg(Pbf)-Glu (OtBu)-Lys(Boc)-Ser(tBu)-Ser(tBu)-NH—(CH₂)₂—O—(CH₂)₂—O—(CH₂)₂—NH—CO—(CH₂)₁₆—CO—O-Wang-polystyrene (for acylation with Fmoc-Dab(Mtt)-OH, Procedure 12 is recommended), then selective removal of Mtt and 2-phenylisopropyl with subsequent lactam cyclization according to Procedure 4, cleavage from the resin and side-chain deprotection according to Procedure 8, reductive alkylation according to Procedure 11, HPLC purification and freeze-drying.

LCMS (system 3): Rt=3.73 min; ((M+2)/2)=1104.9

### Example 10

### Ac-c[Orn-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂

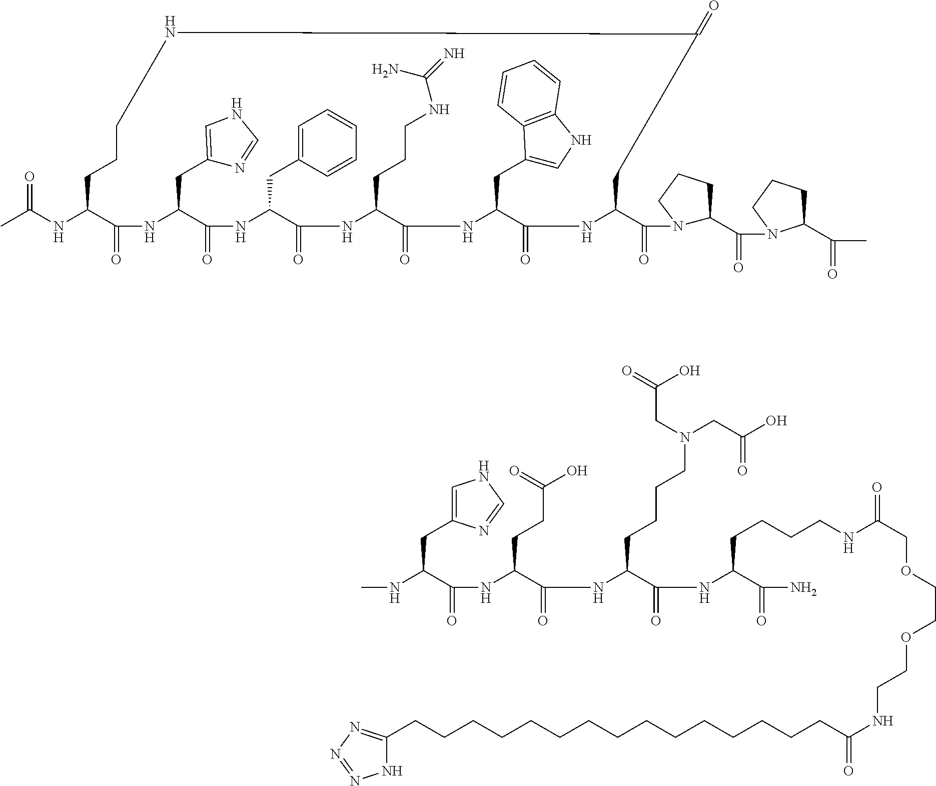 [SMILES: CC(=O)N[C@H]1CCCNC(=O)C[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene, then according to Example 6.

LCMS (system 1b): Rt=2.01 min; ((M+2)/2)=1092

### Example 11

### Ac-c[Lys-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

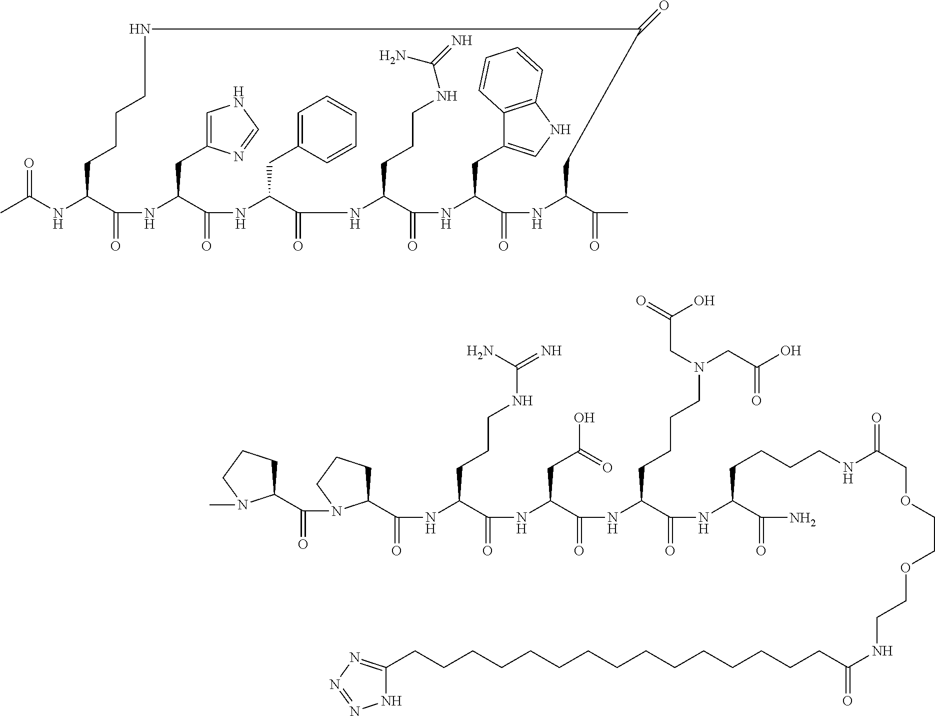 [SMILES: CC(=O)N[C@H]1CCCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Lys(Mtt)-His(Trt)-D-Phe-Arg(Pbf)-Trp(Boc)-Asp(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 1b): Rt=2.03 min; ((M+2)/2)=1101.1

### Example 12

### Ac-c[Orn-His-D-Phe-Arg-Trp-Asp]-Pro-Ser-Ser-Ser-Lys(biscarboxymethyl)-Ser-Ser-Lys({2-[2-(15-carboxypentadecanoylamino)ethoxy]ethoxy}acetyl)-NH₂

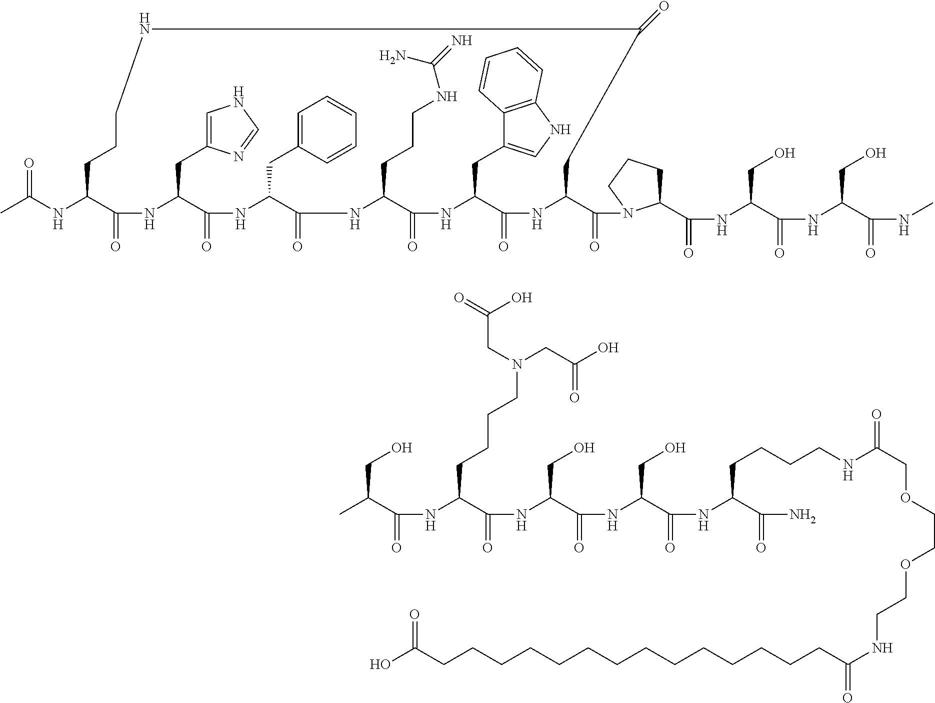

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Ser(tBu)-Ser(tBu)-Ser(tBu)-Lys(Boc)-Ser(tBu)-Ser(tBu)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with hexadecanedioic acid mono-tert-butyl ester acid+HOBt/DIC, cleavage from the resin and side-chain deprotection according to Procedure 8, reductive alkylation according to Procedure 11, HPLC purification and freeze-drying.

LCMS (system 3): Rt=3.45 min; ((M+2)/2)=1108.8

### Example 13

### Ac-c[Glu-His-D-Phe-Arg-Trp-Orn]-Pro-Pro-Asp-Lys(biscarboxymethyl)-Lys({2-[2-(15-carboxypentadecanoylamino)ethoxy]ethoxy}acetyl)-NH₂

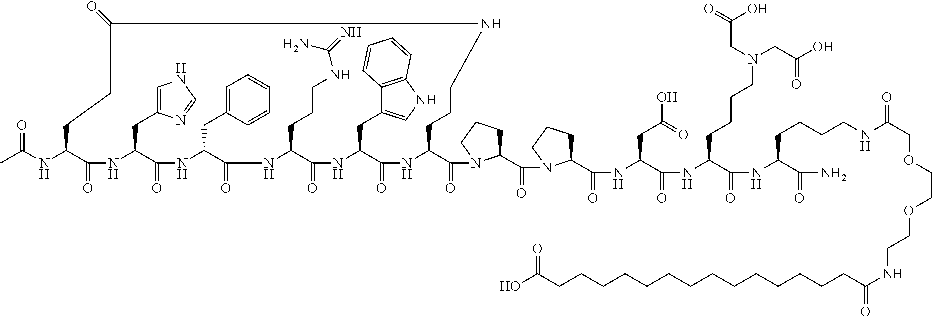 [SMILES: CC(=O)N[C@H]1CCC(=O)NCCC[C@@H](C(=O)N2CCC[C@H]2C(=O)N2CCC[C@H]2C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O]

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-Asp(OtBu)-Lys(Boc)-Lys(ivDde)-NH-RinkAM-polystyrene, then according to Example 12.

LCMS (system 1b): Rt=2.18 min; ((M+2)/2)=1004.0

### Example 14

### Ac-c[Orn-His-D-Phe-Arg-Trp-Asp]-Pro-Ser-Ser-Ser-Lys(biscarboxymethyl)-Ser-Ser-Lys({2-[2-(17-carboxyheptadecanoylamino)ethoxy]ethoxy}acetyl)-NH₂

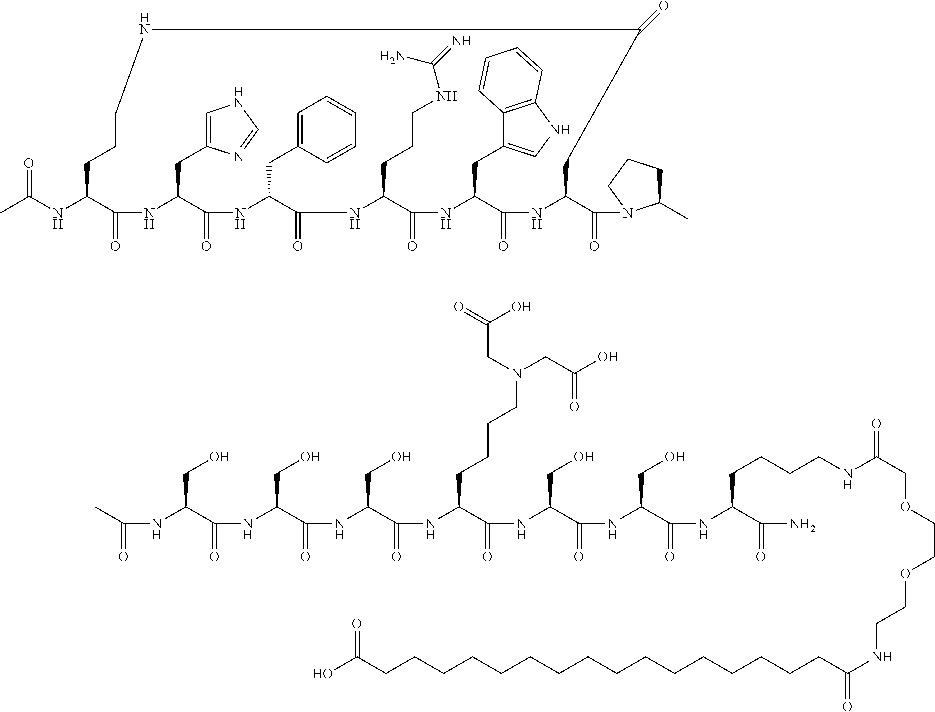

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Ser(tBu)-Ser(tBu)-Ser(tBu)-Lys(Boc)-Ser(tBu)-Ser(tBu)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with octadecanedioic acid mono-tert-butyl ester acid (available by the synthetic procedure described in: U. Widmer, Synthesis 1983, 135)+HOBt/DIC, cleavage from the resin and side-chain deprotection according to Procedure 8, reductive alkylation according to Procedure 11, HPLC purification and freeze-drying.

LCMS (system 1b): Rt=2.39 min; ((M+2)/2)=1123

### Example 15

### Ac-c[Dab-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂

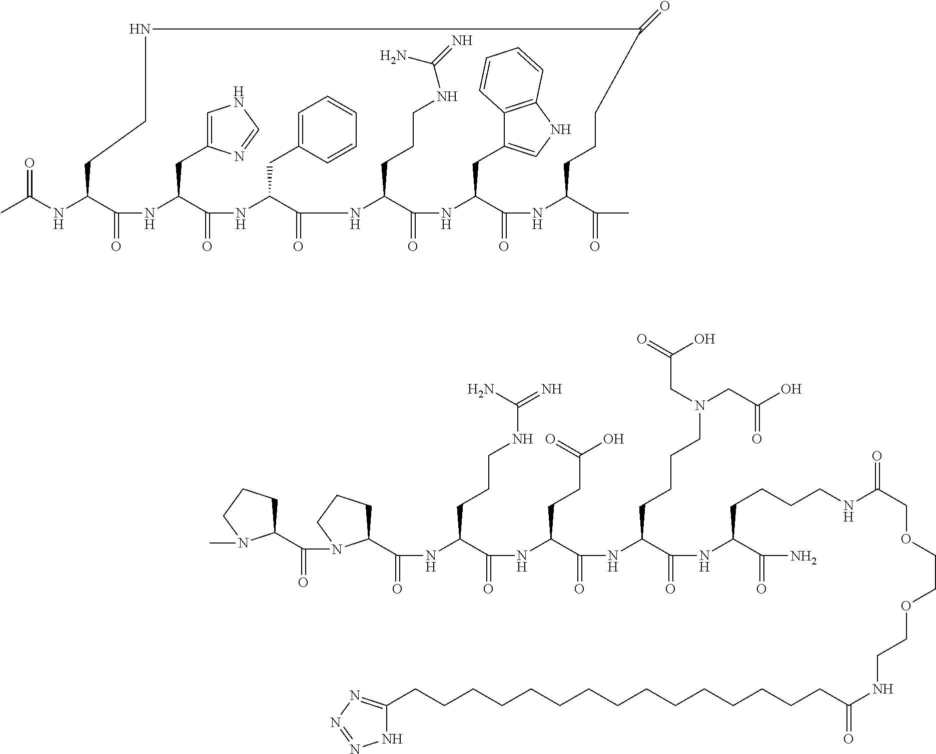 [SMILES: CC(=O)N[C@H]1CCNC(=O)CCC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Dab(Mtt)-His(Trt)-D-Phe-Arg(Pbf)-Trp(Boc)-Asp(2-phenylisopropyloxy)-Pro-Pro-Arg(Pbf)-Glu(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 1a): Rt=1.83 min; ((M+2)/2)=1094.0

### Example 16

### Ac-c[Glu-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

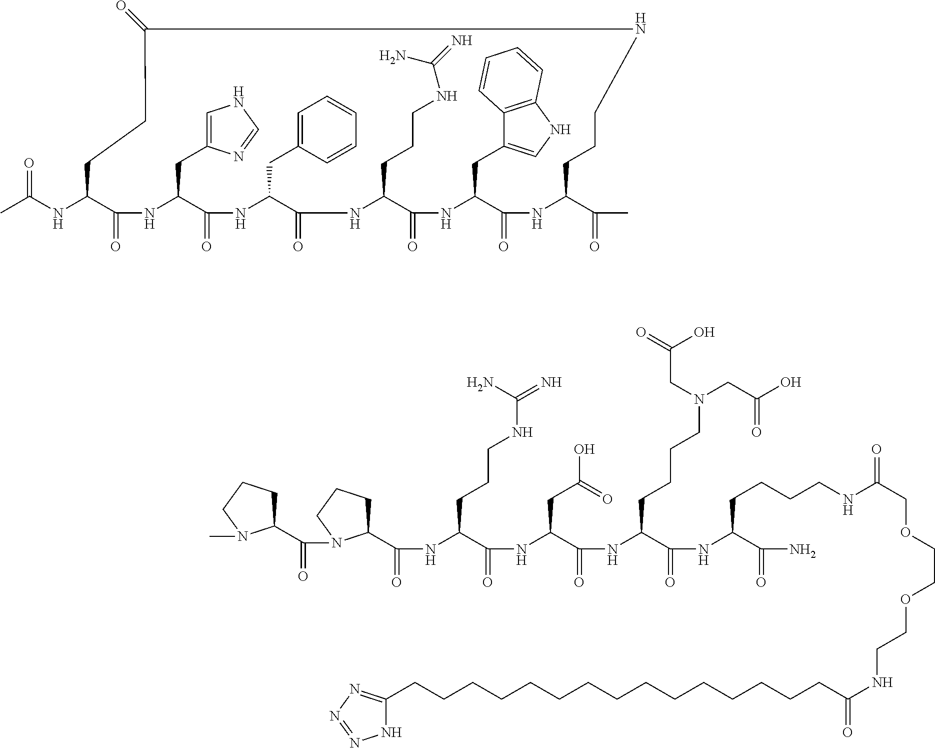 [SMILES: CC(=O)N[C@H]1CCC(=O)NCCC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Dab(Mtt)-Pro-Pro-Arg(Pbf)-Asp(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 1b): Rt=2.13 min; ((M+2)/2)=1094.1

### Example 17

### Ac-c[Orn-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

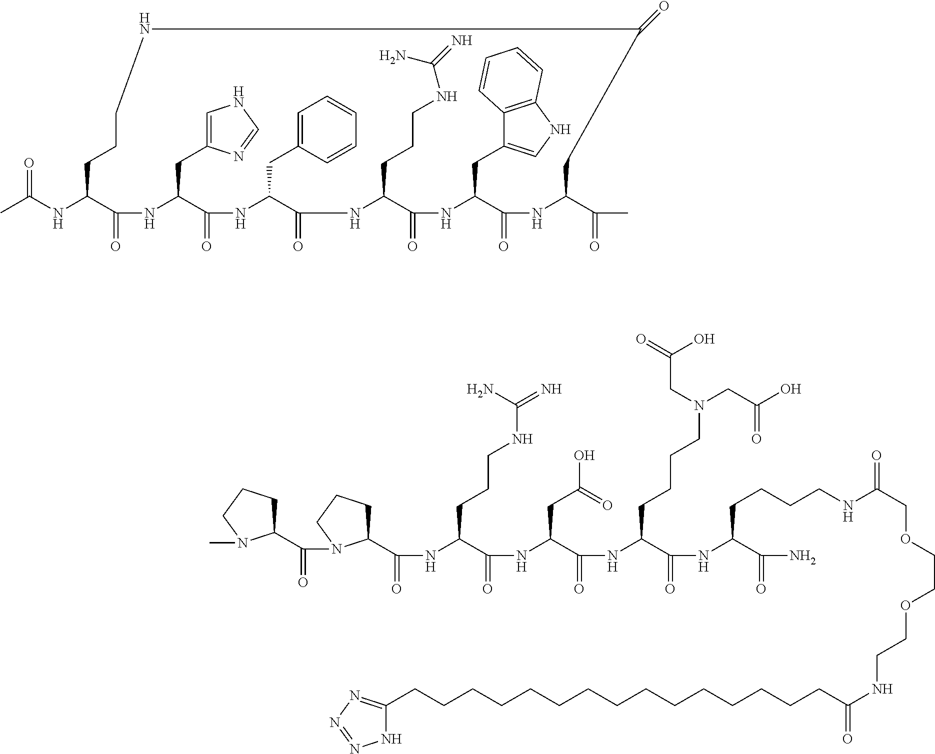 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 2): Rt=2.96 min; ((M+2)/2)=1094.0

### Example 18

### Ac-c[Glu-His-D-Phe-Arg-Trp-Orn]-Pro-Pro-Arg-Asp-Lys(biscarboxymethyl)-Lys({2-[2-(15-carboxypentadecanoylamino)ethoxy]ethoxy}acetyl)-NH₂

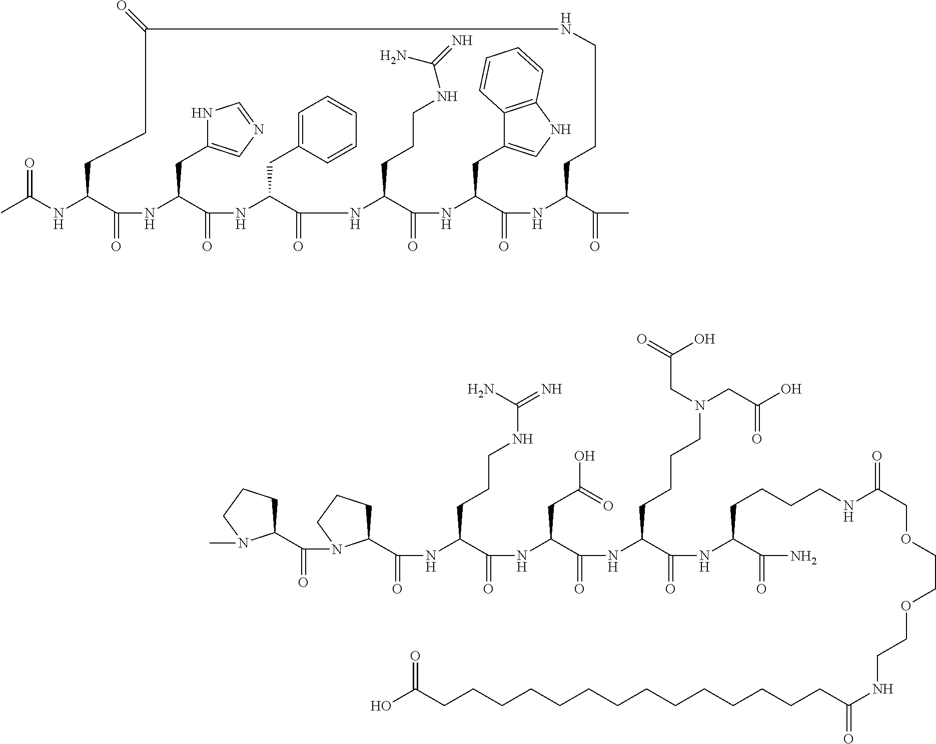

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-Arg(Pbf)-Asp(OtBu)-Lys(Boc)-Lys(ivDde)-NH-RinkAM-polystyrene, then according to Example 12.

LCMS (system 1b): Rt=2.53 min; ((M+2)/2)=1081.9

### Example 19

### Ac-c[Orn-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂

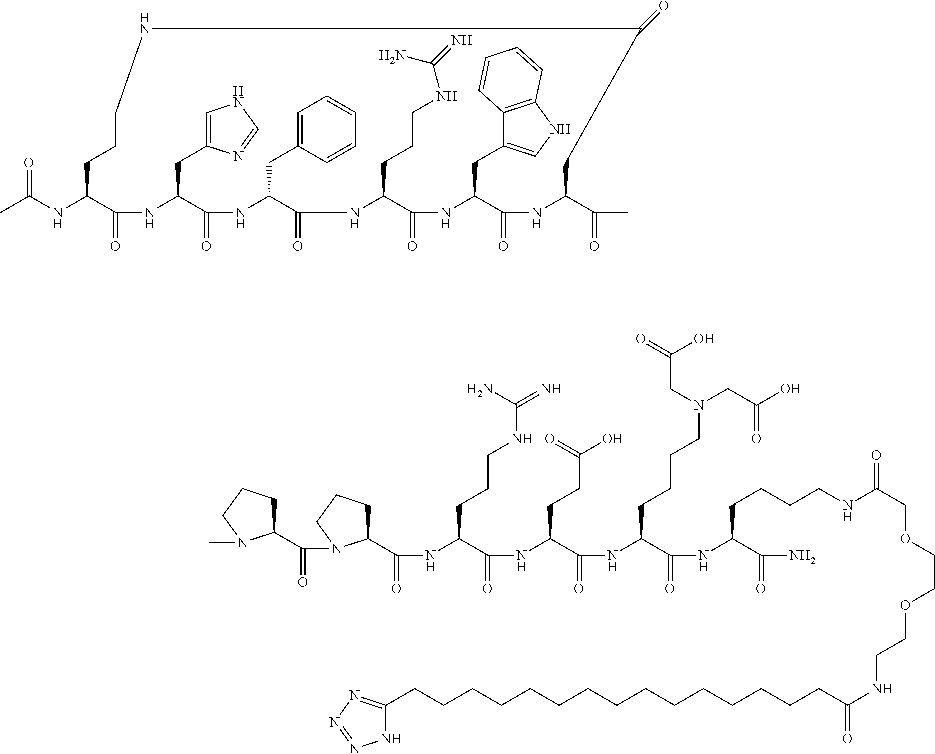 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-Arg(Pbf)-Glu(OtBu)-Lys(Boc)-Lys(ivDde)-NH-RinkAM-polystyrene, then according to Example 6.

LCMS (system 1b): Rt=2.06 min; ((M+2)/2)=1101

### Example 20

### Ac-c[Orn-His-D-Phe-Arg-Trp-Glu]-Pro-Ser-Lys-Glu-Glu-Ser-Ser-Glu-Lys({2-[2-(16-carboxyhexadecanoylamino)ethoxy]ethoxy}acetyl)-NH₂

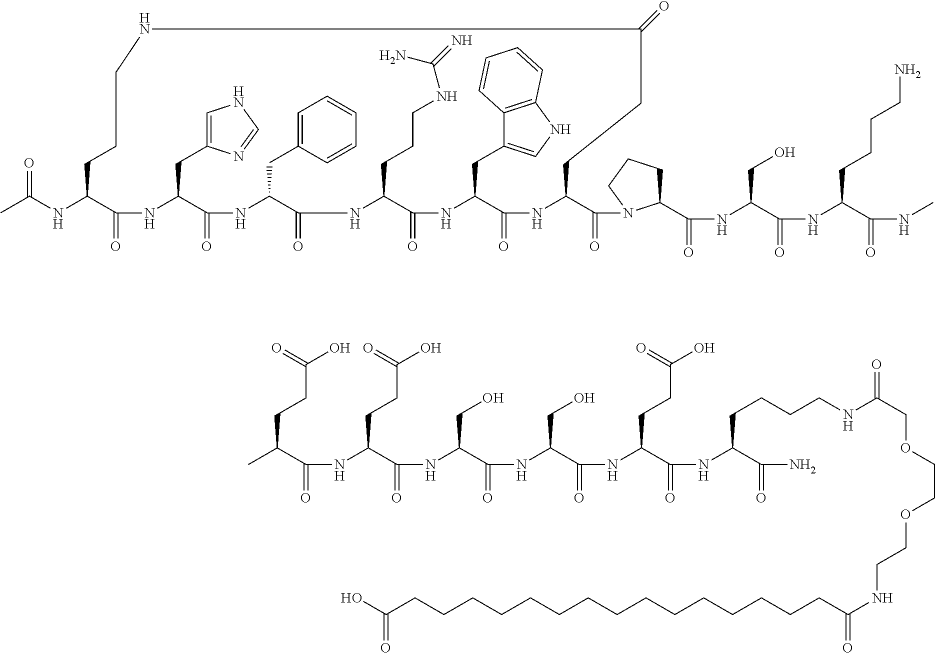

The compound can be prepared by the following synthetic route: Fmoc-based synthesis of peptide resin Ac-Orn(Mtt)-His(Trt)-D-Phe-Arg(Pbf)-Trp(Boc)-Glu(2-phenylisopropyloxy)-Pro-Ser(tBu)-Lys(Boc)-Glu(OtBu)-Glu(OtBu)-Ser(tBu)-Ser(tBu)-Glu(OtBu)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with heptadecanedioic acid mono-tert-butyl ester+HOBt/DIC, cleavage from the resin and side-chain deprotection according to Procedure 8, HPLC purification and freeze-drying.

LCMS (system 3): Rt=3.70 min; ((M+2)/2)=1171.4

### Example 21

### Ac-c[Lys-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

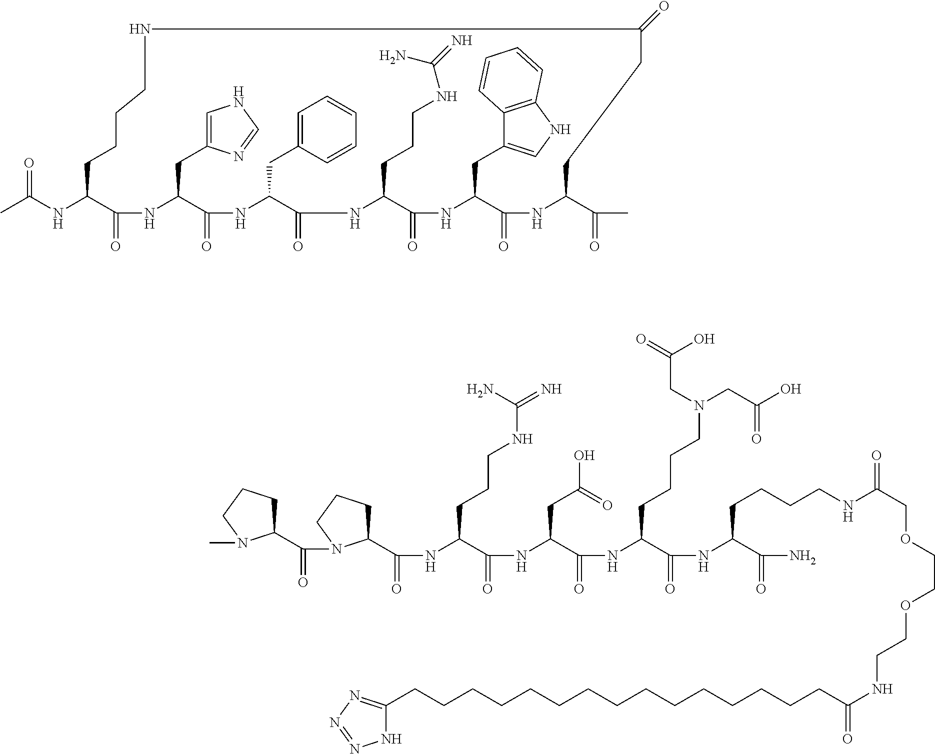 [SMILES: CC(=O)N[C@H]1CCCCNC(=O)CC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Lys(Mtt)-His(Trt)-D-Phe-Arg(Pbf)-Trp(Boc)-Glu(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 3): Rt=3.38 min; ((M+2)/2)=1108.3

### Example 22

### Ac-c[Orn-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂

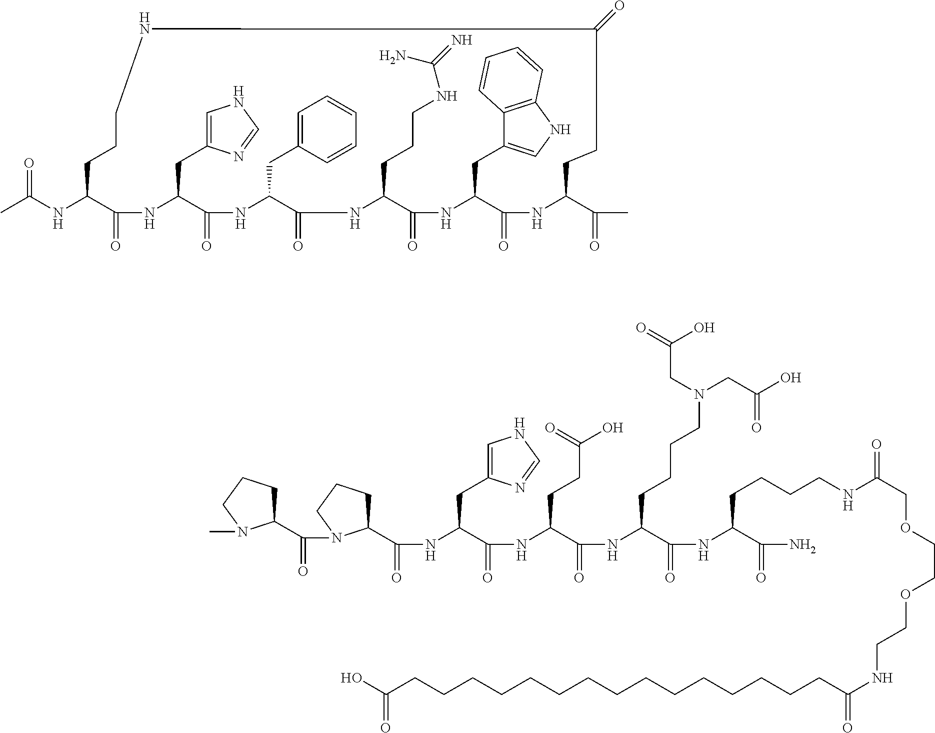

The compound can be prepared by the following synthetic route: Fmoc-based synthesis of peptide resin Ac-Orn(Mtt)-His(Trt)-D-Phe-Arg(Pbf)-Trp(Boc)-Glu(2-phenylisopropyloxy)-Pro-Pro-His(Trt)-Glu(OtBu)-Lys(Boc)-Lys(ivDde)-NH-RinkAM-polystyrene, then according to Example 7.

LCMS (system 1b): Rt=2.08 min; ((M+2)/2)=1087

### Example 23

### Ac-c[Dab-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

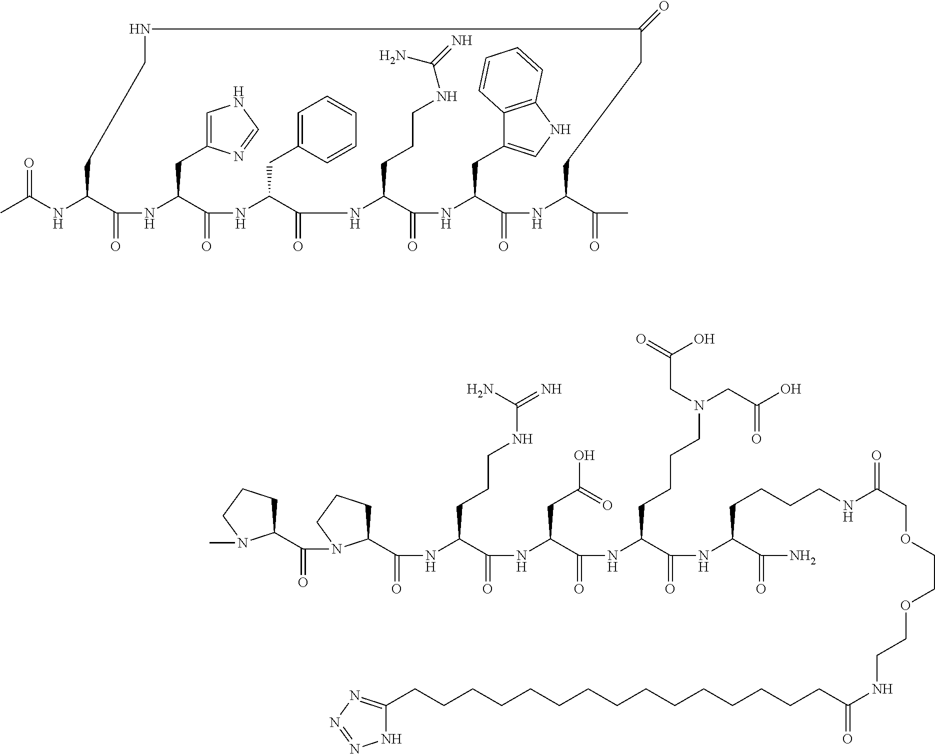 [SMILES: CC(=O)N[C@H]1CCNC(=O)CC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Dab(Mtt)-His(Trt)-D-Phe-Arg(Pbf)-Trp(Boc)-Glu(2-phenylisopropyloxy)-Pro-Pro-Arg(Pbf)-Asp(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 3): Rt=3.35 min; ((M+2)/2)=1094.1

### Example 24

### Ac-c[Asp-His-D-Phe-Arg-Trp-Dap]-Pro-Pro-Arg-Asp-Lys(BCMA)-Lys[(2-{2-[16-(tetrazol-5-yl)hexadecanoylamino]ethoxy}ethoxy)acetyl]-NH₂

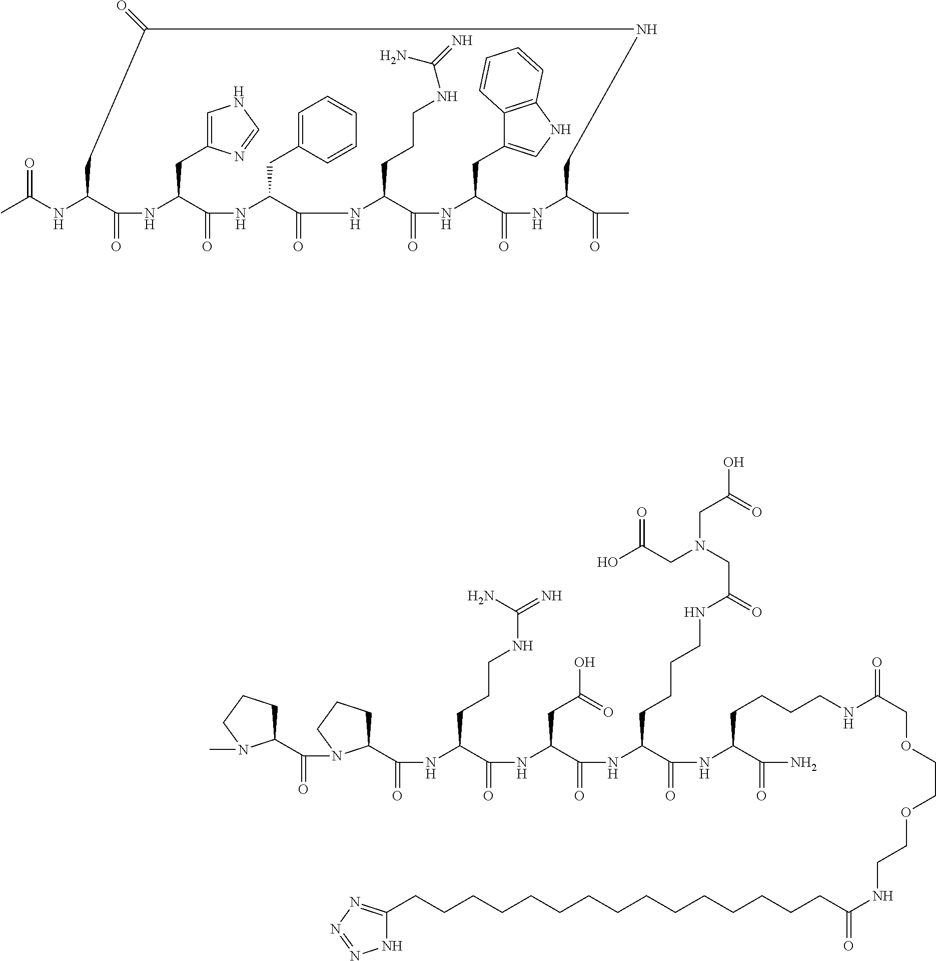 [SMILES: CC(=O)N[C@H]1CC(=O)NC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Asp(2-phenylisopropyloxy)-His(Trt)-D-Phe-Arg(Pbf)-Trp(Boc)-Dap(Mtt)-Pro-Pro-Arg(Pbf)-Asp(OtBu)-Lys(Boc)-Lys(ivDde)-NH-RinkAM-polystyrene, then selective removal of Mtt and 2-phenylisopropyl with subsequent lactam cyclization according to Procedure 4, removal of ivDde according to Procedure 1, acylation with Fmoc-8-amino-3,6-dioxaoctanoic acid+HOBt/DIC, deprotection with piperidine in NMP, acylation with 16-(tetrazol-5-yl)hexadecanoic acid+HOBt/DIC, cleavage from the resin and side-chain deprotection according to Procedure 7, acylation with bis(tert-butoxycarbonylmethyl)aminoacetic acid and subsequent removal of tBu according to Procedure 13, HPLC purification and freeze-drying.

LCMS (system 1a): Rt=1.87 min; ((M+2)/2)=1108.5

### Example 25

### Ac-c[Dab-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

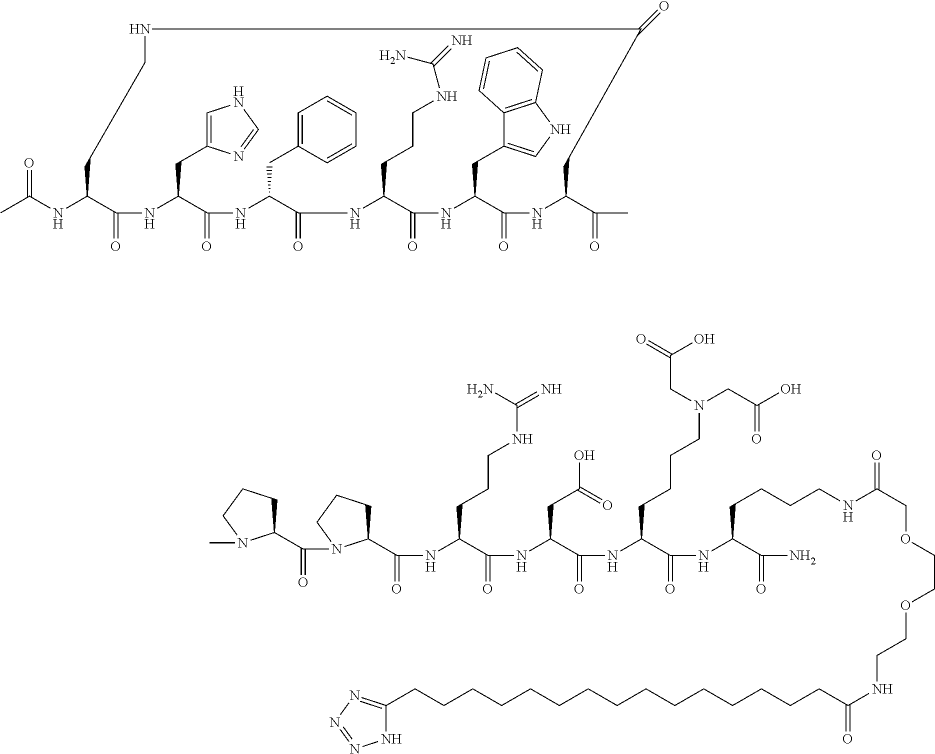 [SMILES: CC(=O)N[C@H]1CCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Dab(Mtt)-His(Trt)-D-Phe-Arg(Pbf)-Trp(Boc)-Asp(2-phenylisopropyloxy)-Pro-Pro-Arg(Pbf)-Asp(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 1b): Rt=1.87 min; ((M+2)/2)=1087.1

### Example 26

### Ac-c[Glu-His-D-Phe-Arg-Trp-Orn]-Pro-Pro-Asp-Lys(biscarboxymethyl)-Lys({2-[2-(17-carboxyheptadecanoylamino)ethoxy]ethoxy}acetyl)-NH₂

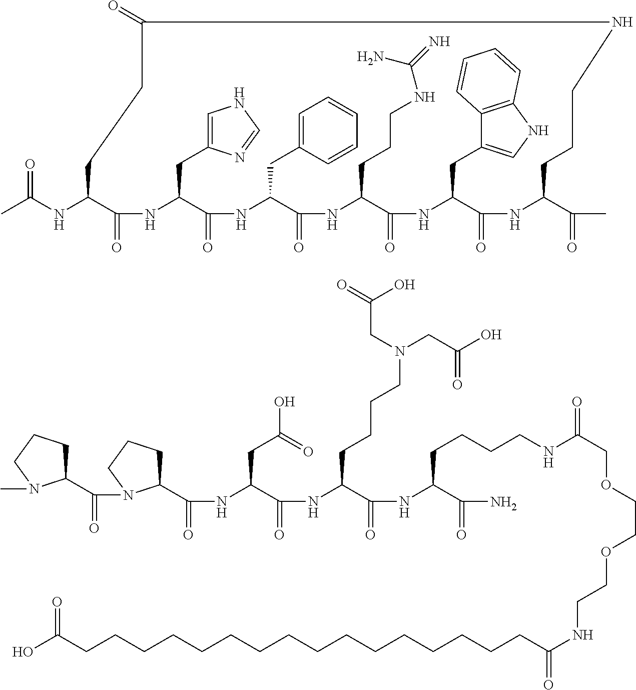

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-Asp(OtBu)-Lys(Boc)-Lys(ivDde)-NH-RinkAM-polystyrene, then according to Example 14.

LCMS (system 1b): Rt=2.37 min; ((M+2)/2)=1018.0

### Example 27

### Ac-c[Glu-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

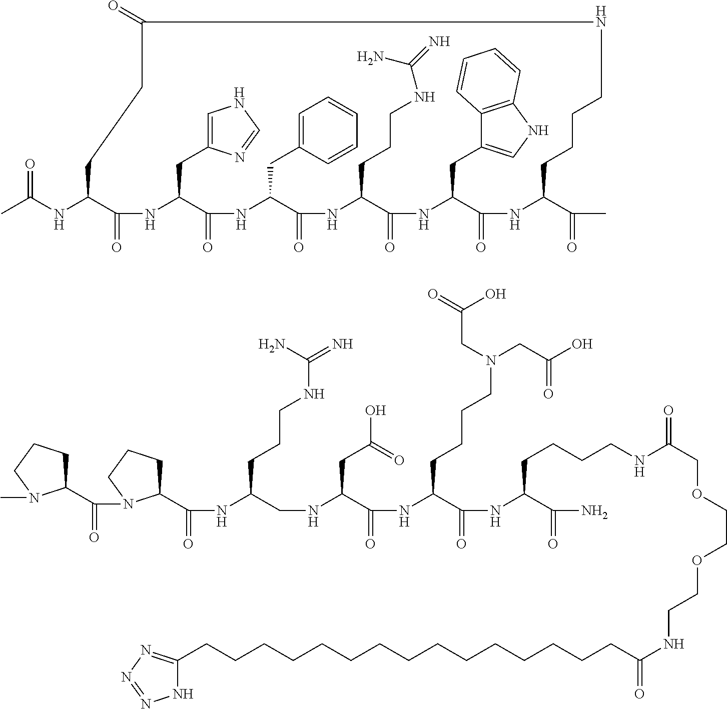

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Lys(Mtt)-Pro-Pro-Arg(Pbf)-Asp(OtBu)-Lys(Boc)-Lys(ivDde)-NH-RinkAM-polystyrene, then according to Example 6.

LCMS (system 1b): Rt=2.17 min; ((M+2)/2)=1108.1

### Example 28

### Ac-c[Orn-His-D-Phe-Arg-Trp-Asp]-Pro-Ser-Ser-Ser-Lys(biscarboxymethyl)-Ser-Ser-Lys[(2-{2-[16-(tetrazol-5-yl)hexadecanoylamino]ethoxy}ethoxy)acetyl]-NH₂

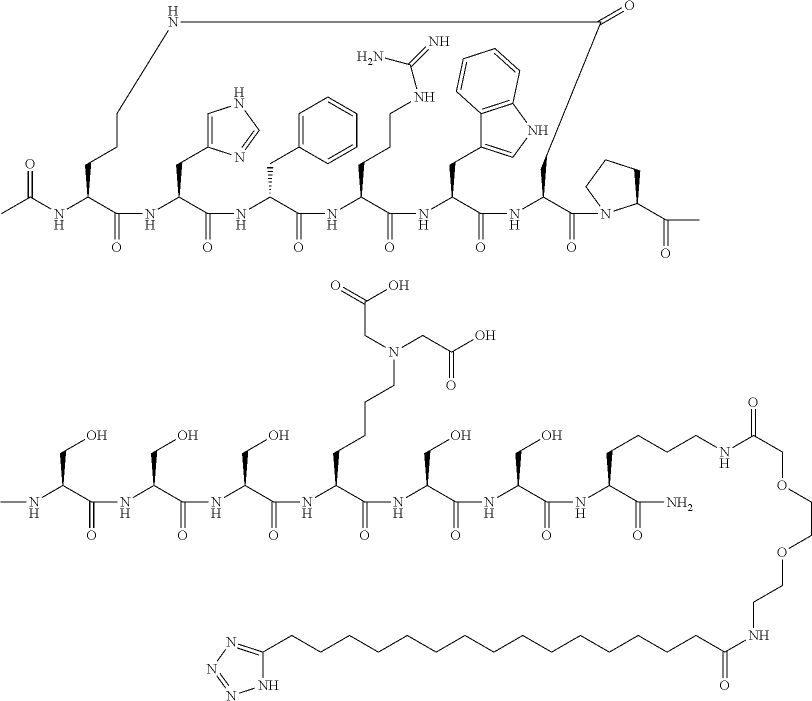

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Ser(tBu)-Ser(tBu)-Ser(tBu)-Lys(Boc)-Ser(tBu)-Ser(tBu)-Lys(ivDde)-NH-RinkAM-polystyrene, then according to Example 6.

LCMS (system 3): Rt=3.44 min; ((M+2)/2)=1127.8

### Example 29

### Ac-c[Glu-His-D-Phe-Arg-Trp-Orn]-D-Pro-D-Pro-Arg-Asp-Lys(biscarboxymethyl)-Lys[(2-{2-[16-(tetrazol-5-yl)hexadecanoylamino]ethoxy}ethoxy)acetyl]-NH₂

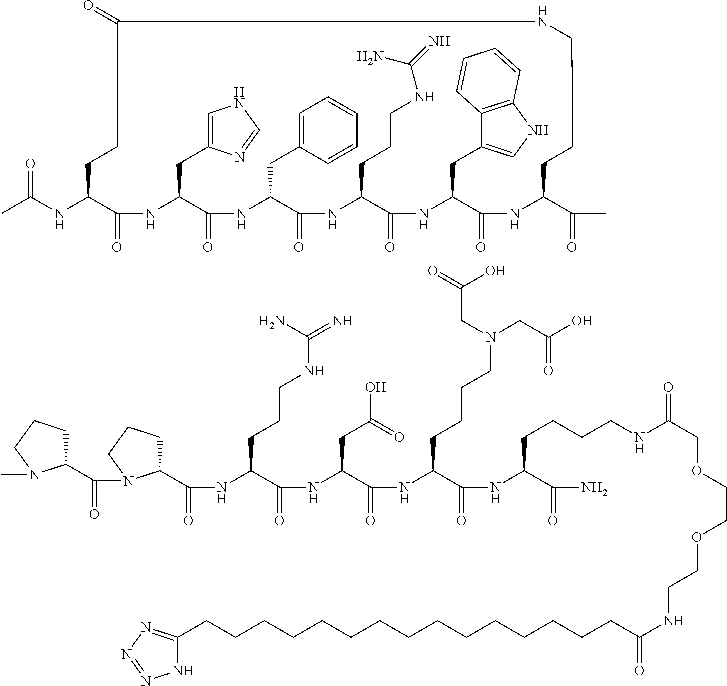

The compound can be prepared by the following synthetic route: Fmoc-based synthesis of peptide resin Ac-Glu (2-phenyl isopropyloxy)-His(Trt)-D-Phe-Arg(Pbf)-Trp(Boc)-Orn(Mtt)-D-Pro-D-Pro-Arg(Pbf)-Asp(OtBu)-Lys(Boc)-Lys(ivDde)-NH-RinkAM-polystyrene, then according to Example 6.

LCMS (system 1b): Rt=1.75 min; ((M+2)/2)=1101.1

### Example 30

### Ac-c[Asp-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

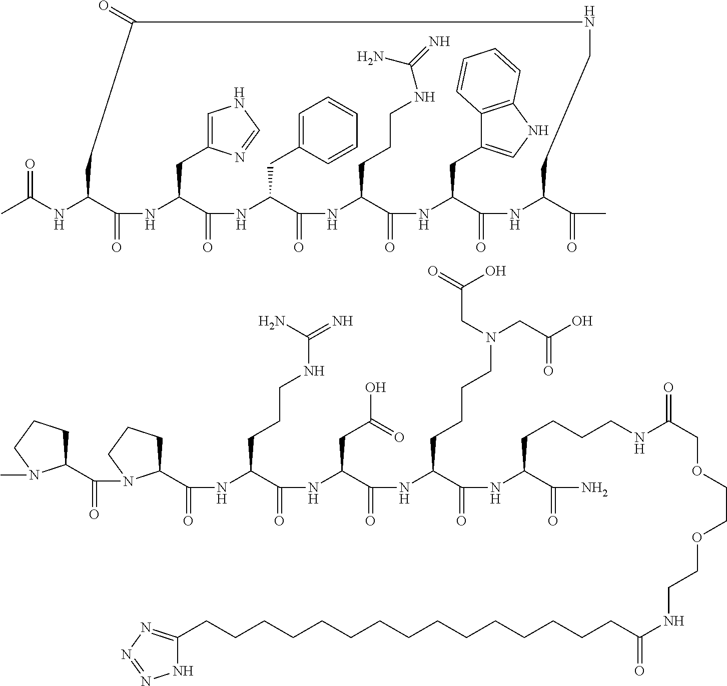

The compound can be prepared by the following synthetic route: Fmoc-based synthesis of peptide resin Ac-Asp(2-phenylisopropyloxy)-His(Trt)-D-Phe-Arg(Pbf)-Trp(Boc)-Dab(Mtt)-Pro-Pro-Arg(Pbf)-Asp(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 1b): Rt=1.89 min; ((M+2)/2)=1087.1

### Example 31

### Ac-c[Glu-His-D-Phe-Arg-Trp-Dap]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

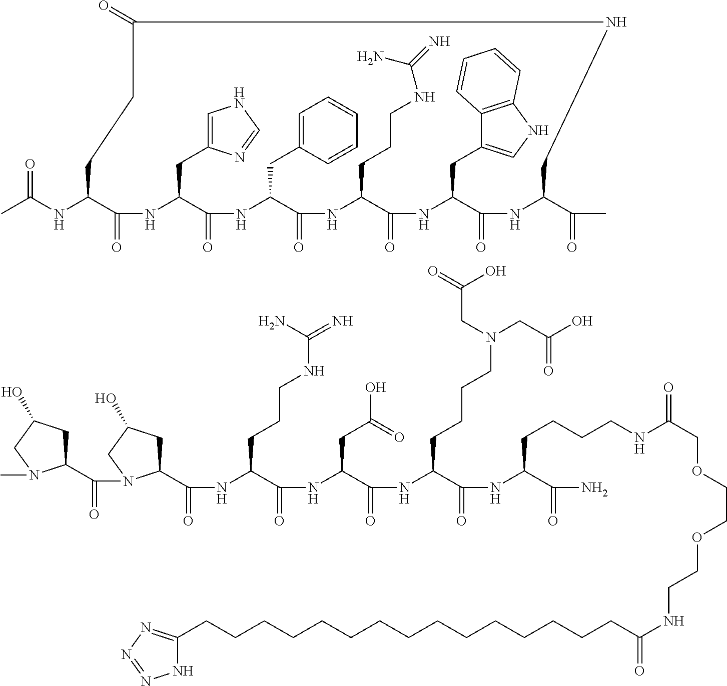

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Dap(Mtt)-Hyp(tBu)-Hyp(tBu)-Arg(Pbf)-Asp(OtBu)-Lys(Boc)-Lys(ivDde)-NH-RinkAM-polystyrene, then according to Example 6.

LCMS (system 1b): Rt=1.99 min; ((M+2)/2)=1103.0

### Example 32

### Ac-c[Glu-His-D-Phe-Arg-Trp-Dab]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

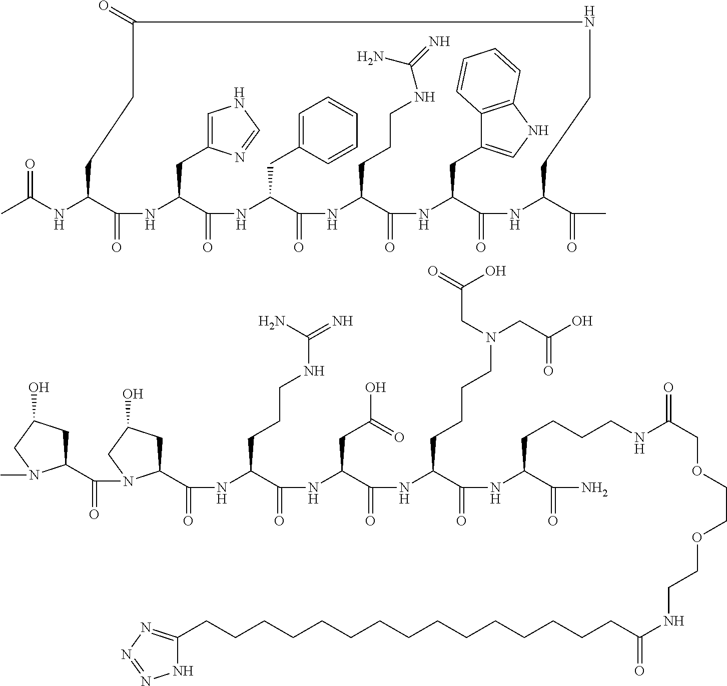

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Dab(Mtt)-Hyp(tBu)-Hyp(tBu)-Arg(Pbf)-Asp(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 1b): Rt=2.08 min; ((M+2)/2)=1110.1

### Example 33

### Ac-c[Dab-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂

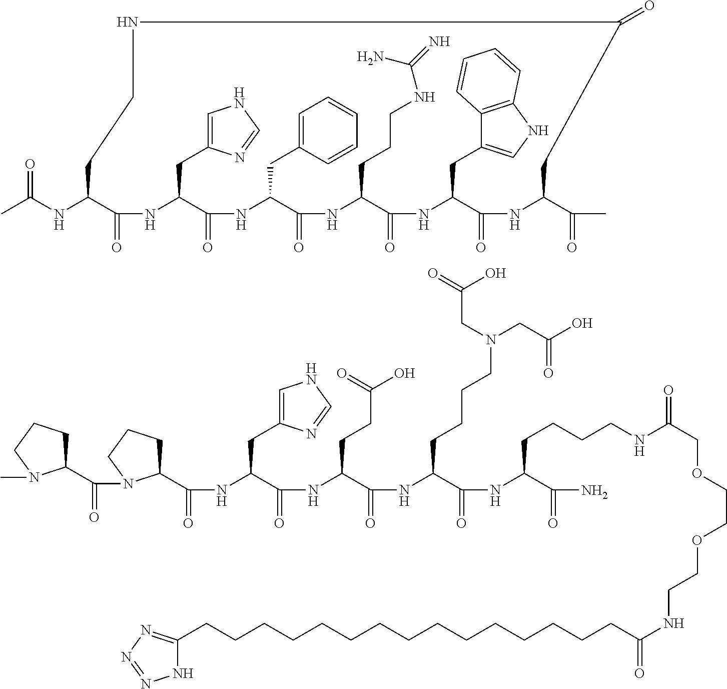

The compound can be prepared by the following synthetic route: Fmoc-based synthesis of peptide resin Ac-Dab(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene (for acylation with Fmoc-Dab(Mtt)-OH, Procedure 12 is recommended), then according to Example 6.

LCMS (system 3): Rt=3.51 min; ((M+2)/2)=1084.6

### Example 34

### Ac-c[Orn-His-D-Phe-Arg-Trp-Asp]-Pro-Pro-Arg-Glu-Lys(biscarboxymethyl)-Lys({2-[2-(17-carboxyheptadecanoylamino)ethoxy]ethoxy}acetyl)-NH₂

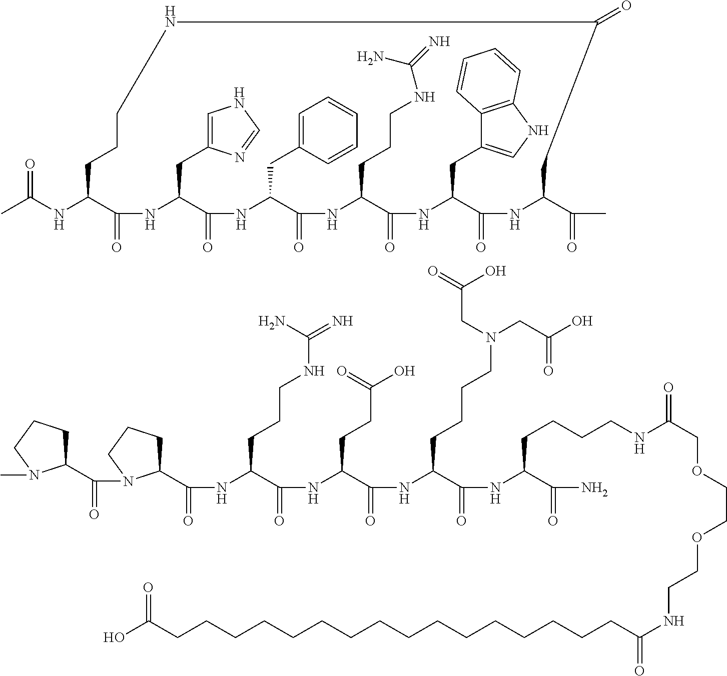

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-Arg(Pbf)-Glu(OtBu)-Lys(Boc)-Lys(ivDde)-NH-RinkAM-polystyrene, then according to Example 14.

LCMS (system 3): Rt=3.79 min; ((M+2)/2)=1096.8

### Example 35

### Ac-c[Asp-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

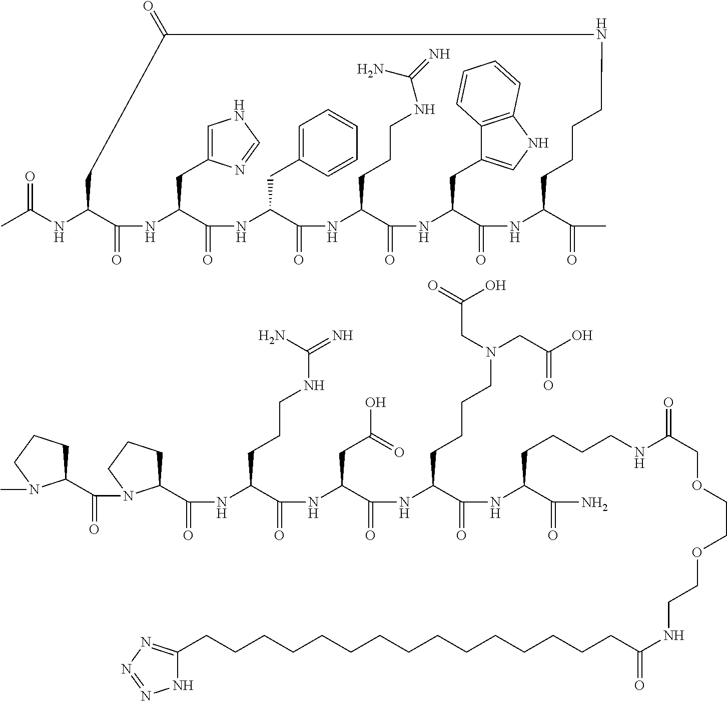

The compound can be prepared by the following synthetic route: Fmoc-based synthesis of peptide resin Ac-Asp(2-phenylisopropyloxy)-His(Trt)-D-Phe-Arg(Pbf)-Trp(Boc)-Lys(Mtt)-Pro-Pro-Arg(Pbf)-Asp(OtBu)-Lys(Boc)-Lys(ivDde)-NH-RinkAM-polystyrene, then according to Example 6.

LCMS (system 1b): Rt=2.13 min; ((M+2)/2)=1101.1

### Example 36

### Ac-c[Dap-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

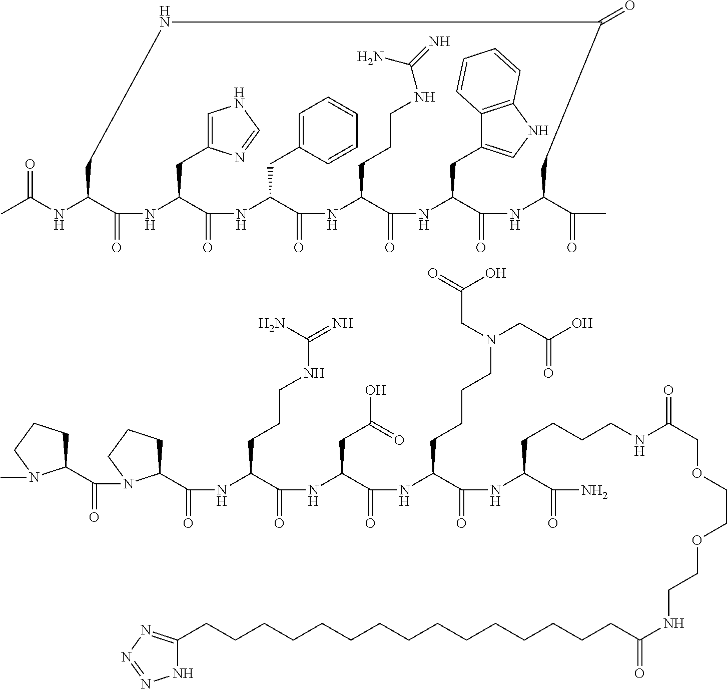

The compound can be prepared by the following synthetic route: Fmoc-based synthesis of peptide resin Ac-Dap(Mtt)-His(Trt)-D-Phe-Arg(Pbf)-Trp(Boc)-Asp(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 1b): Rt=1.89 min; ((M+2)/2)=1080.0

### Example 37

### Ac-c[Glu-Gln-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

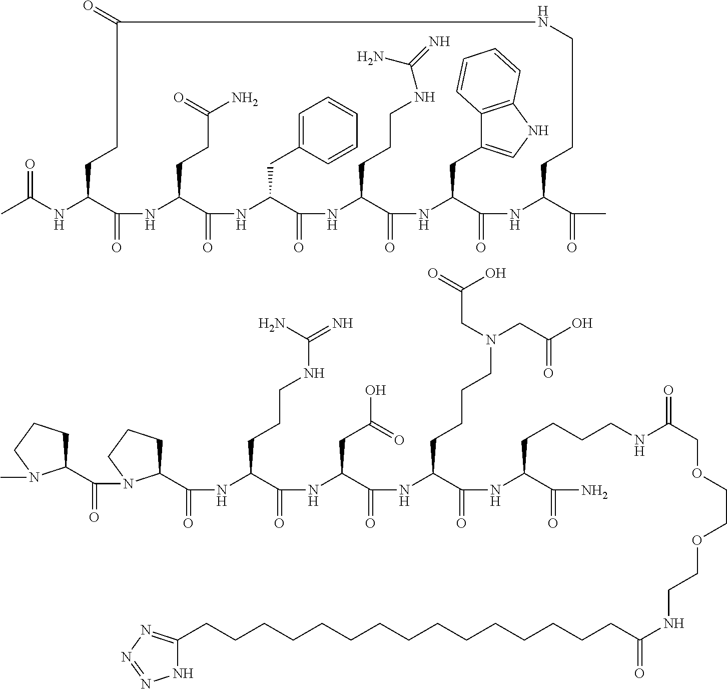

The compound can be prepared by the following synthetic route: Fmoc-based synthesis of peptide resin Ac-Glu(2-phenylisopropyloxy)-Gln(Trt)-D-Phe-Arg(Pbf)-Trp(Boc)-Orn(Mtt)-Pro-Pro-Arg(Pbf)-Asp(OtBu)-Lys(Boc)-Lys(ivDde)-NH-RinkAM-polystyrene, then according to Example 6.

LCMS (system 1b): Rt=2.79 min; ((M+2)/2)=1096.4

### Example 38

### Ac-c[Glu-His-D-Phe-Arg-Trp-Orn]-Pro-Pro-Arg-Asp-Lys(biscarboxymethyl)-Lys({2-[2-(17-carboxyheptadecanoylamino)ethoxy]ethoxy}acetyl)-NH₂

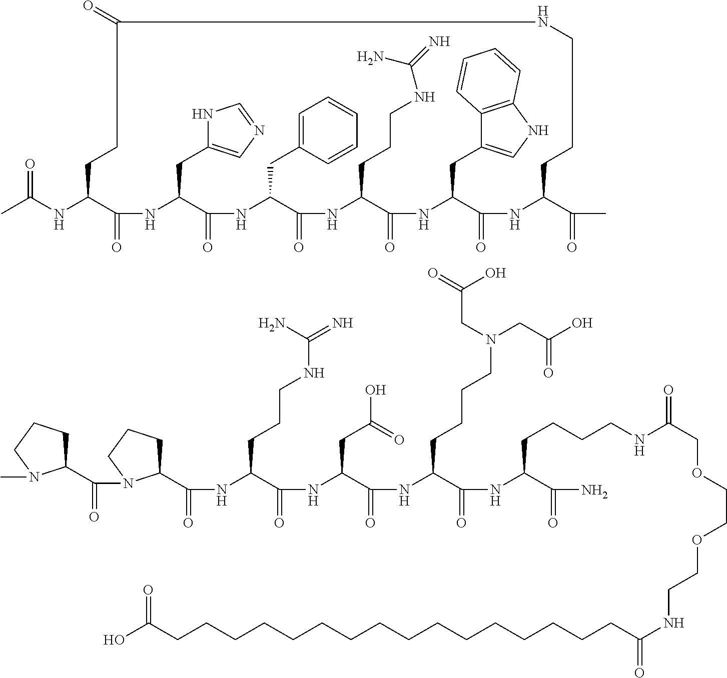

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-Arg(Pbf)-Asp(OtBu)-Lys(Boc)-Lys(ivDde)-NH-RinkAM-polystyrene, then according to Example 14.

LCMS (system 1b): Rt=2.03 min; ((M+2)/2)=1096.0

### Example 39

### Ac-c[Orn-His-D-Phe-Arg-Trp-Glu]-Pro-Ser-Ser-Ser-Glu-Glu-Ser-Ser-Lys({2-[2-(16-carboxyhexadecanoylamino)ethoxy]ethoxy}acetyl)-NH₂

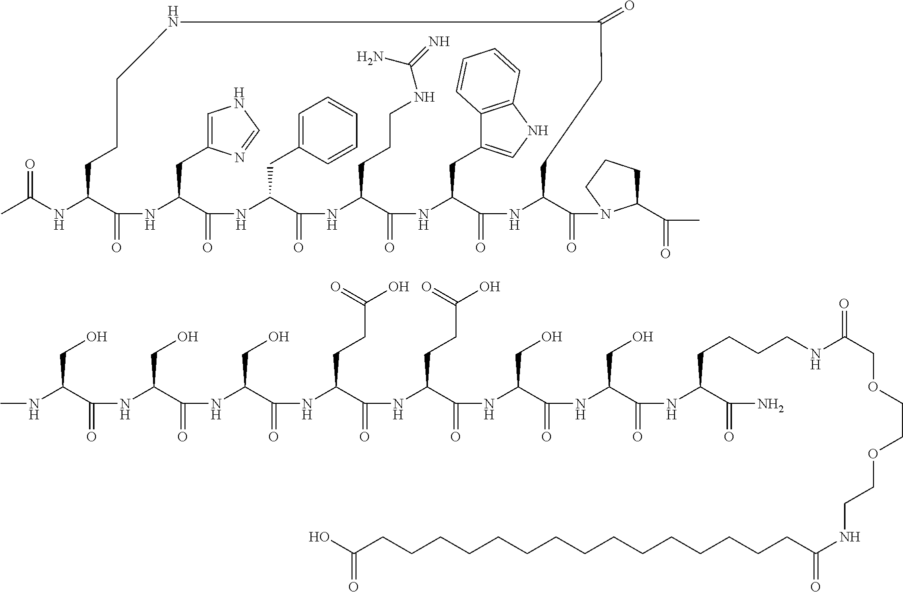

The compound can be prepared by the following synthetic route: Fmoc-based synthesis of peptide resin Ac-Orn(Mtt)-His(Trt)-D-Phe-Arg(Pbf)-Trp(Boc)-Glu(2-phenylisopropyloxy)-Pro-Ser(tBu)-Ser(tBu)-Ser(tBu)-Glu(OtBu)-Glu(OtBu)-Ser(tBu)-Ser(tBu)-Lys(ivDde)-NH-RinkAM-polystyrene, then according to Example 20.

LCMS (system 1b): Rt=2.13 min; ((M+2)/2)=1129.5

### Example 40

### Ac-c[Orn-Glu-His-D-Phe-Arg-Trp-Gly-Asp]-Pro-Ser-Lys-Glu-Glu-Ser-Ser-Lys({2-[2-(16-carboxyhexadecanoylamino)ethoxy]ethoxy}acetyl)-NH₂

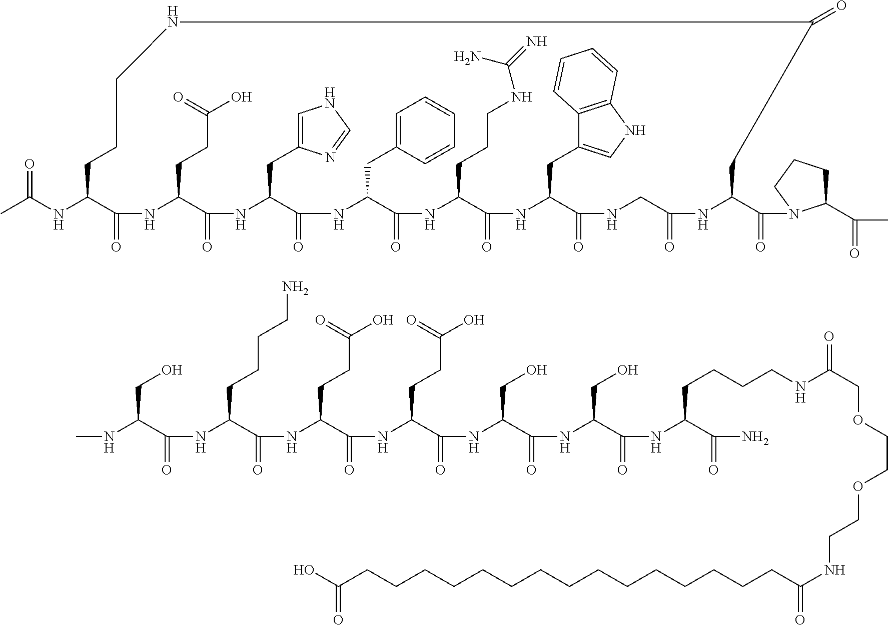

The compound can be prepared by the following synthetic route: Fmoc-based synthesis of peptide resin Ac-Orn(Mtt)-Glu(OtBu)-His(Trt)-D-Phe-Arg(Pbf)-Trp(Boc)-Gly-Asp(2-phenylisopropyloxy)-Pro-Ser(tBu)-Lys(Boc)-Glu(OtBu)-Glu(OtBu)-Ser(tBu)-Ser(tBu)-Lys(ivDde)-NH-RinkAM-polystyrene, then according to Example 20.

LCMS (system 3): Rt=3.67 min; ((M+2)/2)=1192.6

### Example 41

### Ac-c[Asp-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

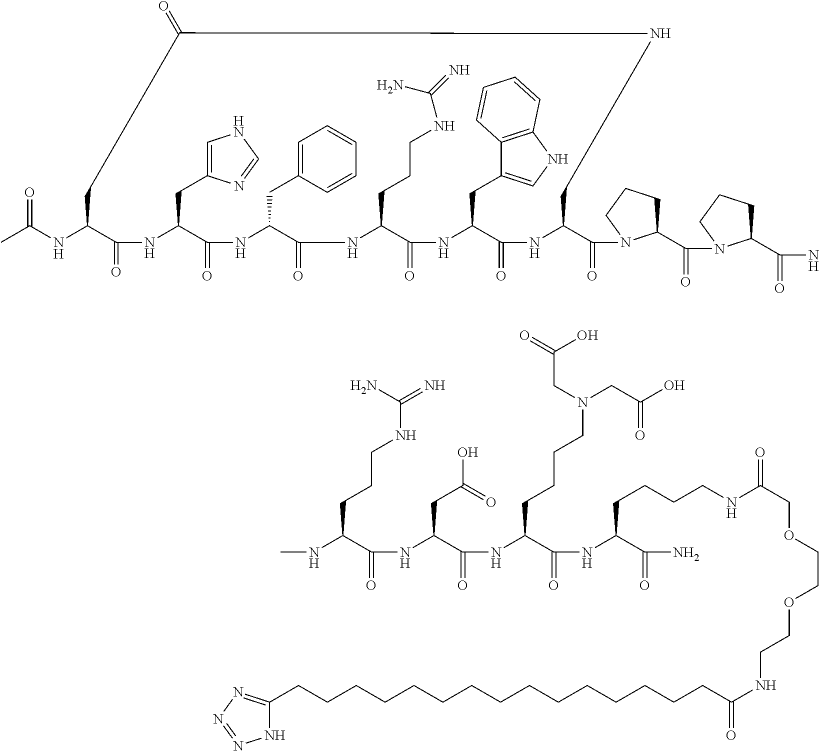

The compound can be prepared by the following synthetic route: Fmoc-based synthesis of peptide resin Ac-Asp(2-phenylisopropyloxy)-His(Trt)-D-Phe-Arg(Pbf)-Trp(Boc)-Dap(Mtt)-Pro-Pro-Arg(Pbf)-Asp(OtBu)-Lys(Boc)-Lys(ivDde)-NH-RinkAM-polystyrene, then according to Example 6.

LCMS (system 1b): Rt=1.98 min; ((M+2)/2)=1080.0

### Example 42

### Ac-c[Orn-His-D-Phe-Arg-Trp-Asp]-Pro-Pro-Arg-Glu-Lys(biscarboxymethyl)-Lys({2-[2-(15-carboxypentadecanoylamino)ethoxy]ethoxy}acetyl)-NH₂

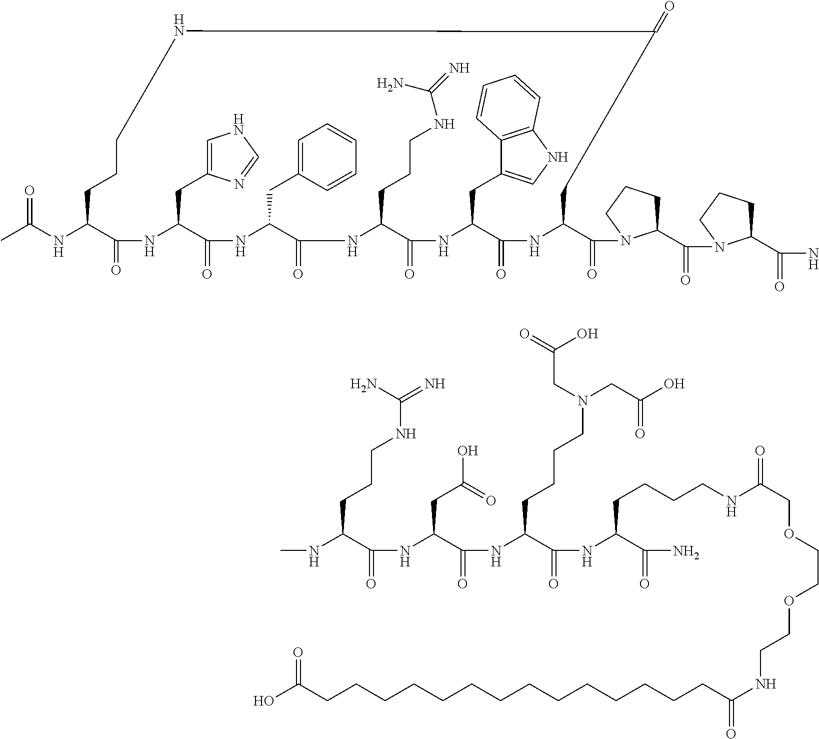

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-Arg(Pbf)-Glu(OtBu)-Lys(Boc)-Lys(ivDde)-NH-RinkAM-polystyrene, then according to Example 12.

LCMS (system 1b): Rt=1.99 min; ((M+2)/2)=1082

### Example 43

### Ac-c[Glu-His-D-Phe-Arg-Trp-Orn]-Pro-Pro-His-Asp-Lys(biscarboxymethyl)-Lys[(2-{2-[16-(tetrazol-5-yl)hexadecanoylamino]ethoxy}ethoxy)acetyl]-NH₂

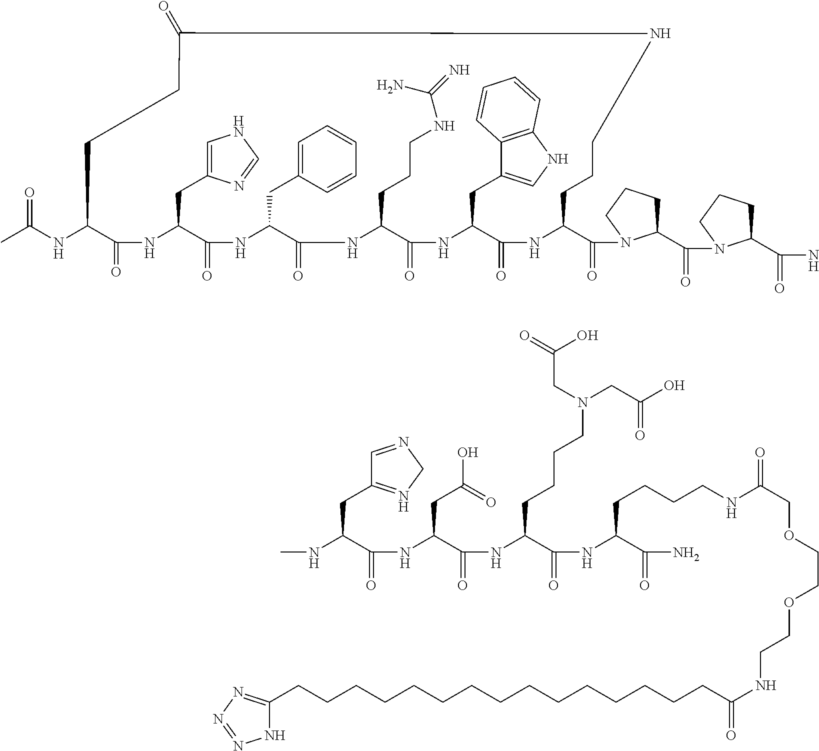

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-His(Trt)-Asp(OtBu)-Lys(Boc)-Lys(ivDde)-NH-RinkAM-polystyrene, then according to Example 6.

LCMS (system 3): Rt=3.48 min; ((M+2)/2)=1091.7

### Example 44

### Ac-c[Lys-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂

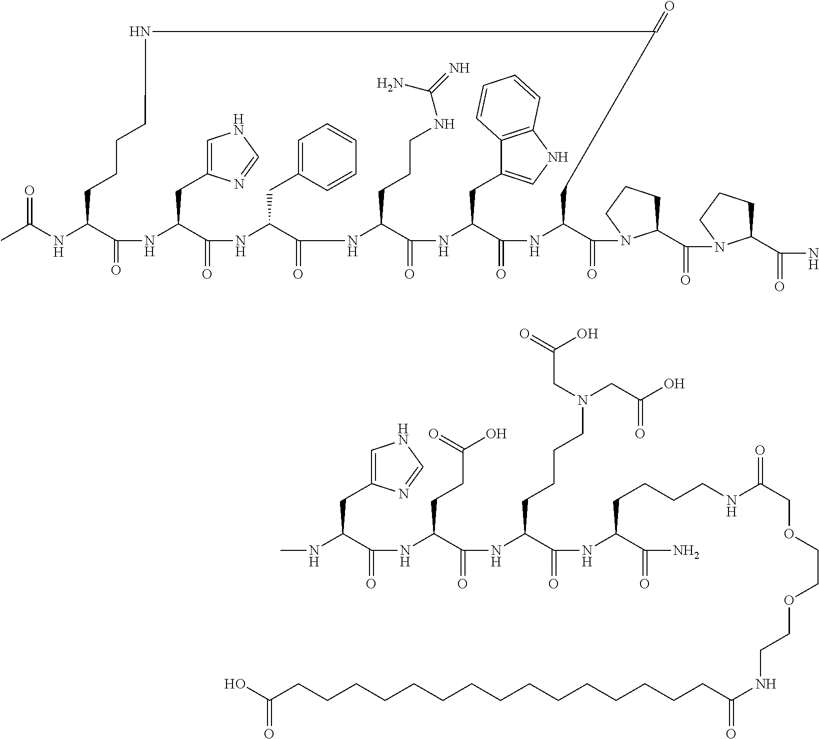

The compound can be prepared by the following synthetic route: Fmoc-based synthesis of peptide resin Ac-Lys(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene, then according to Example 7.

LCMS (system 3): Rt=3.76 min; ((M+2)/2)=1086.6

### Example 45

### Ac-c[Lys-His-D-Phe-Arg-Trp-Asp]-Pro-Pro-His-Ser-Lys(biscarboxymethyl)-Ser-Ser-Glu-Lys({2-[2-(16-carboxyhexadecanoylamino)ethoxy]ethoxy}acetyl)-NH₂

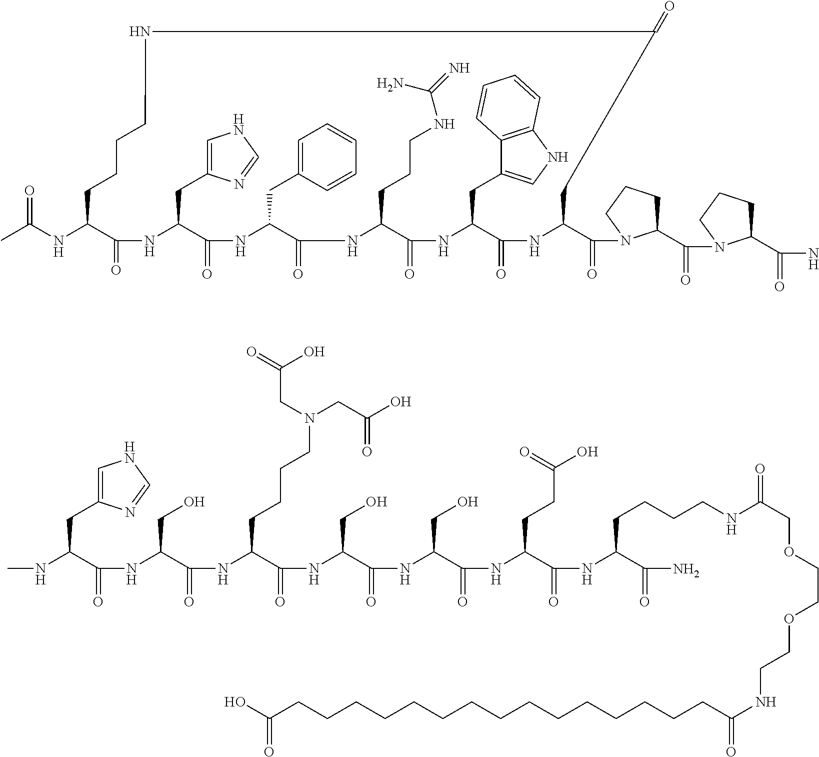

The compound can be prepared by the following synthetic route: Fmoc-based synthesis of peptide resin Ac-Lys(Mtt)-His(Trt)-D-Phe-Arg(Pbf)-Trp(Boc)-Asp(2-phenylisopropyloxy)-Pro-Pro-His(Trt)-Ser(tBu)-Lys(Boc)-Ser(tBu)-Ser(tBu)-Glu(OtBu)-Lys(ivDde)-NH-RinkAM-polystyrene, then according to Example 7.

LCMS (system 3): Rt=3.55 min; ((M+2)/2)=1217.3

### Example 46

### Ac-c[Orn-His-D-Phe-Arg-Trp-Asp]-Pro-Pro-His-Glu-Lys(biscarboxymethyl)-Lys({2-[2-(17-carboxyheptadecanoylamino)ethoxy]ethoxy}acetyl)-NH₂

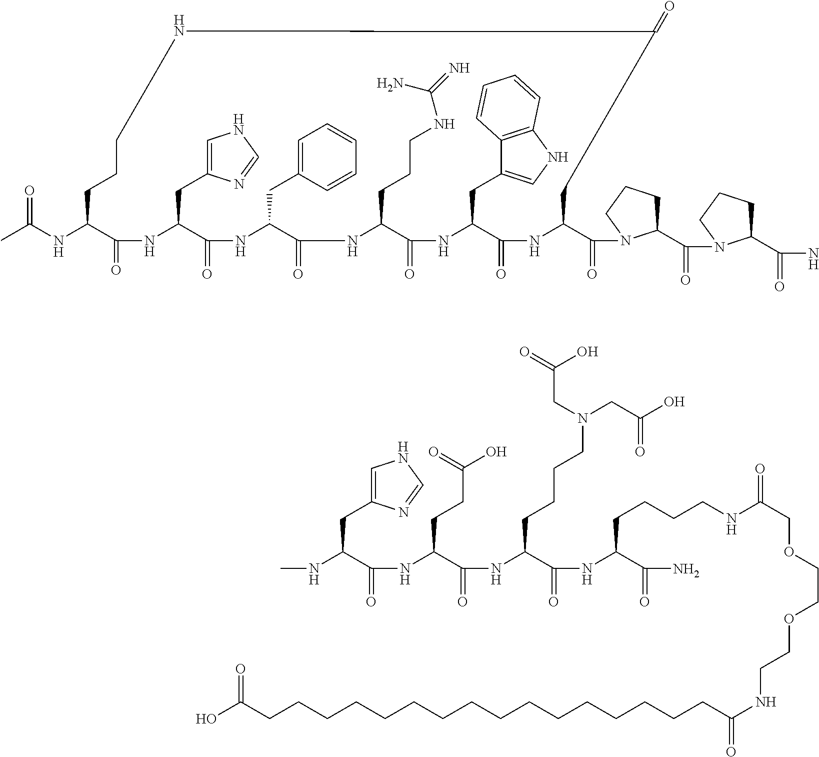

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene, then according to Example 14.

LCMS (system 1b): Rt=2.17 min; ((M+2)/2)=1087

### Example 47

### Ac-c[Dap-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

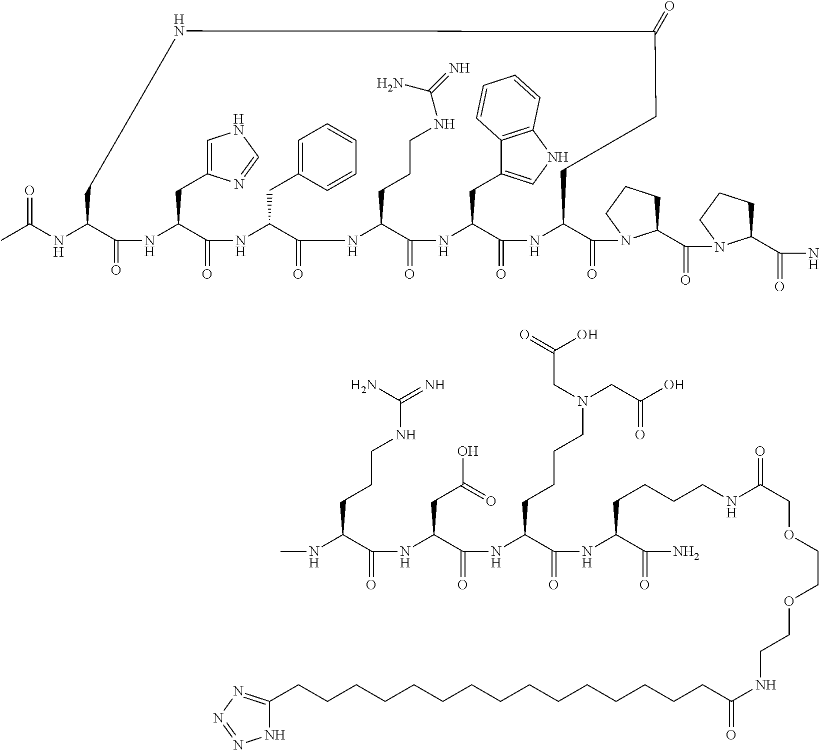

The compound can be prepared by the following synthetic route: Fmoc-based synthesis of peptide resin Ac-Dap(Mtt)-His(Trt)-D-Phe-Arg(Pbf)-Trp(Boc)-Glu(2-phenylisopropyloxy)-Pro-Pro-Arg(Pbf)-Asp(OtBu)-Lys(Boc)-Lys(ivDde)-NH-RinkAM-polystyrene, then according to Example 6.

LCMS (system 1b): Rt=2.06 min; ((M+2)/2)=1087.0

### Example 48

### Ac-c[Glu-His-D-Phe-Arg-Trp-Lys]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

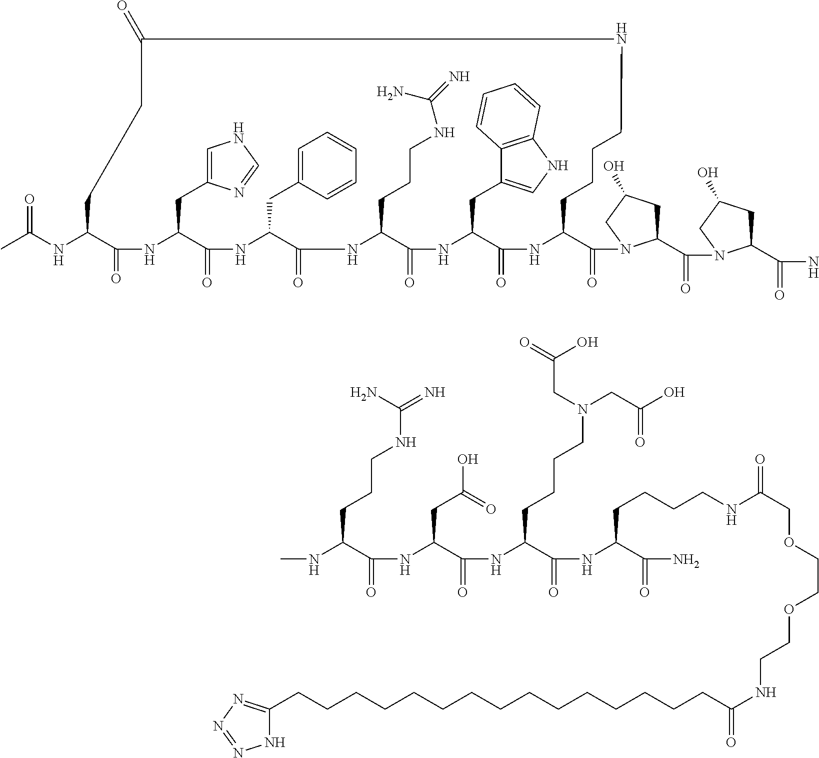

The compound can be prepared by the following synthetic route: Fmoc-based synthesis of peptide resin Ac-Glu (2-phenyl isopropyloxy)-His(Trt)-D-Phe-Arg(Pbf)-Trp(Boc)-Lys(Mtt)-Hyp(tBu)-Hyp(tBu)-Arg(Pbf)-Asp(OtBu)-Lys(Boc)-Lys(ivDde)-NH-RinkAM-polystyrene, then according to Example 6.

LCMS (system 1b): Rt=1.98 min; ((M+2)/2)=1124.1

### Example 49

### Ac-c[Lys-His-D-Phe-Arg-Trp-Asp]-Pro-Ser-His-Glu-Lys(biscarboxymethyl)-Lys({2-[2-(16-carboxyhexadecanoylamino)ethoxy]ethoxy}acetyl)-NH₂

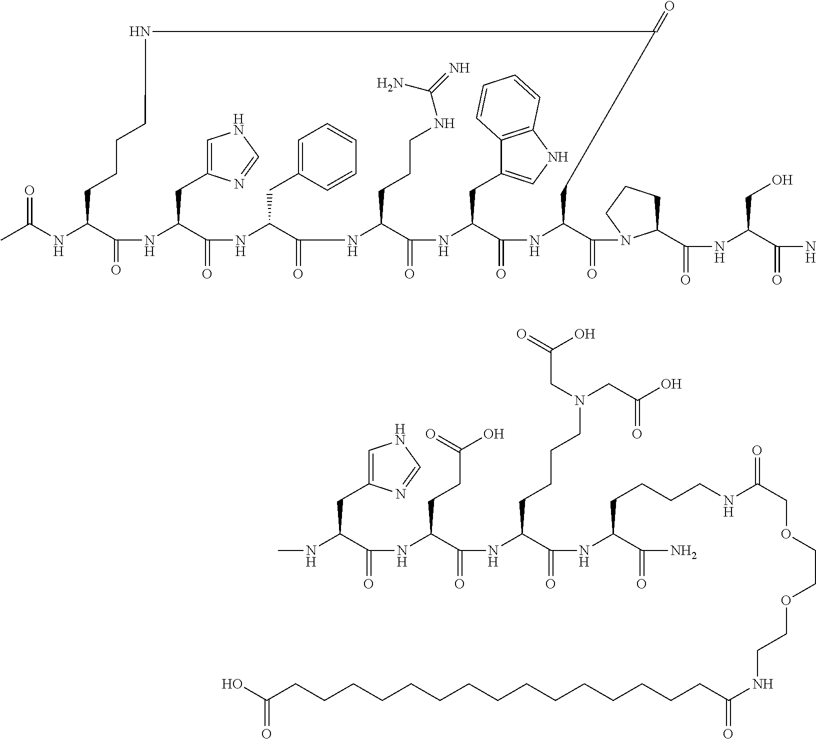

The compound can be prepared by the following synthetic route: Fmoc-based synthesis of peptide resin Ac-Lys(Mtt)-His(Trt)-D-Phe-Arg(Pbf)-Trp(Boc)-Asp(2-phenylisopropyloxy)-Pro-Ser(tBu)-His(Trt)-Glu(OtBu)-Lys(Boc)-Lys(ivDde)-NH-RinkAM-polystyrene, then according to Example 7.

LCMS (system 3): Rt=3.51 min; ((M+2)/2)=1081.6

### Example 50

### Ac-c[Orn-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl)

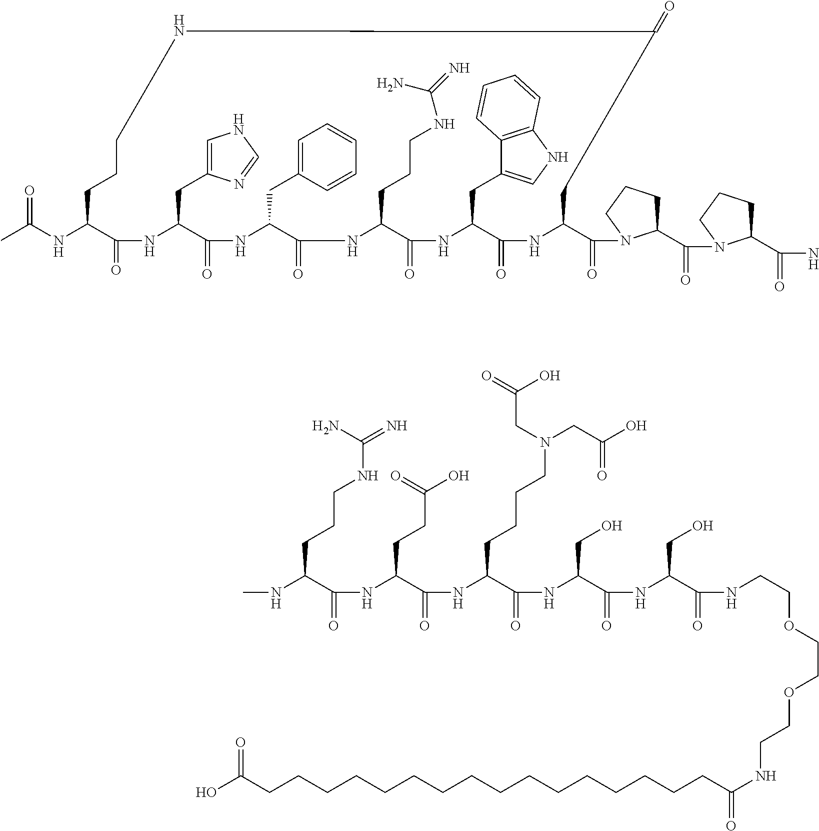

The compound can be prepared by the following synthetic route: preparation of resin Fmoc-Ser(tBu)-NH—(CH₂)₂—O—(CH₂)₂—O—(CH₂)₂—NH—CO—(CH₂)₁₆—CO—O-Wang-polystyrene as described for Example 3, then Fmoc-based synthesis of peptide resin Ac-Orn(Mtt)-His(Trt)-D-Phe-Arg(Pbf)-Trp(Boc)-Asp(2-phenylisopropyloxy)-Pro-Pro-Arg(Pbf)-Glu(OtBu)-Lys(Boc)-Ser(tBu)-Ser(tBu)-NH—(CH₂)₂—O—(CH₂)₂—O—(CH₂)₂—NH—CO—(CH₂)₁₆—CO—O-Wang-polystyrene, then according to Example 9.

LCMS (system 1b): Rt=2.17 min; ((M+2)/2)=1112

### Example 51

### Ac-c[Orn-His-D-Phe-Arg-Trp-Asp]-Pro-Pro-His-Glu-Lys(biscarboxymethyl)-Lys({2-[2-(15-carboxypentadecanoylamino)ethoxy]ethoxy}acetyl)-NH₂

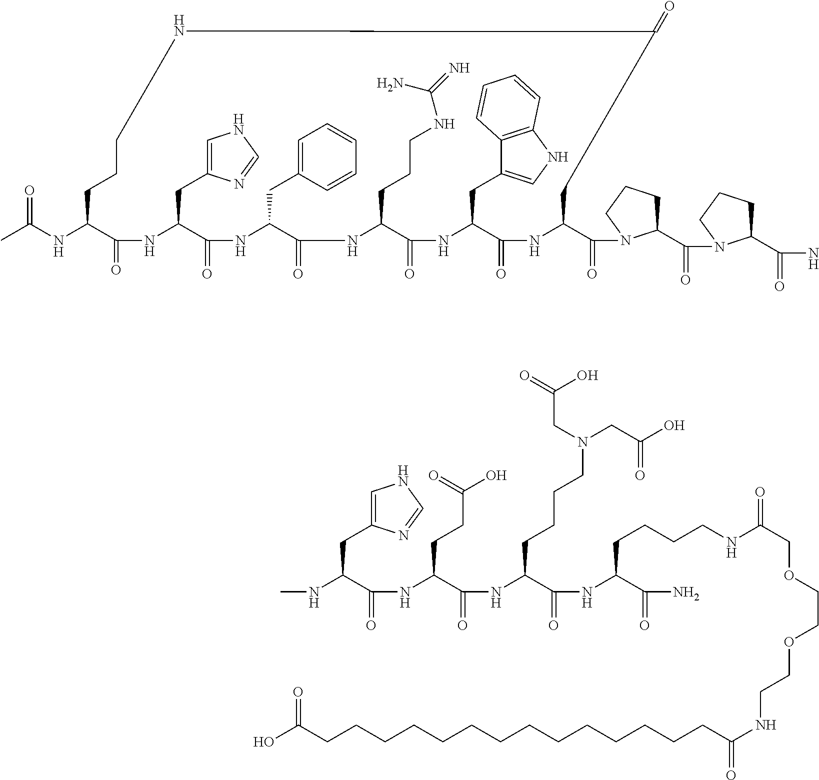

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene, then according to Example 12.

LCMS (system 1b): Rt=1.99 min; ((M+2)/2)=1073

### Example 52

### Ac-c[Lys-His-D-Phe-Arg-Trp-Asp]-Pro-Pro-His-Glu-Glu-Ser-Ser-Glu-Lys({2-[2-(16-carboxyhexadecanoylamino)ethoxy]ethoxy}acetyl)-NH₂

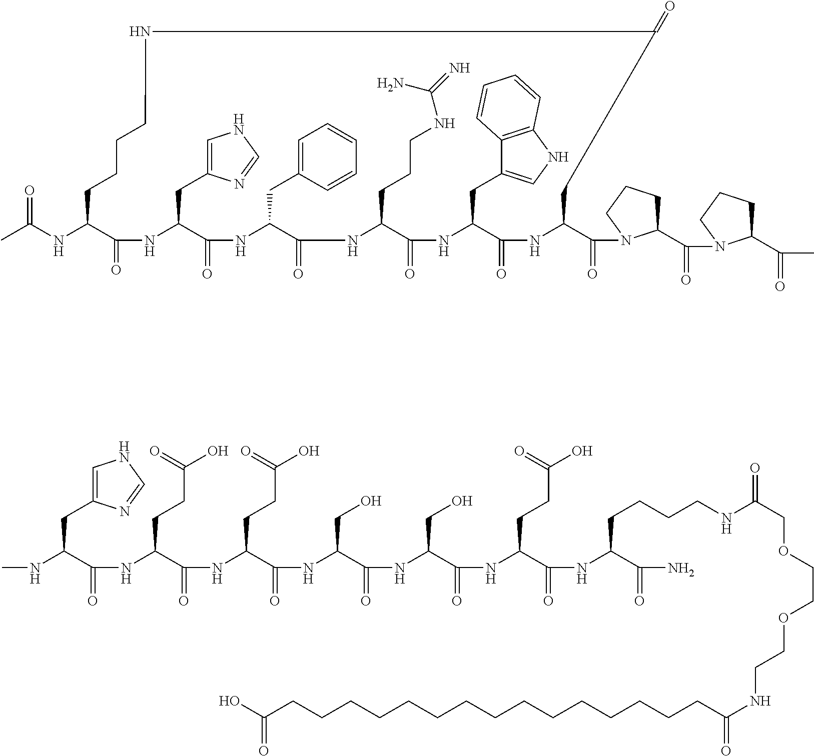

The compound can be prepared by the following synthetic route: Fmoc-based synthesis of peptide resin Ac-Lys(Mtt)-His(Trt)-D-Phe-Arg(Pbf)-Trp(Boc)-Asp(2-phenylisopropyloxy)-Pro-Pro-His(Trt)-Glu(OtBu)-Glu(OtBu)-Ser(tBu)-Ser(tBu)-Glu(OtBu)-Lys(ivDde)-NH-RinkAM-polystyrene, then according to Example 20.

LCMS (system 1b): Rt=2.10 min; ((M+2)/2)=1080.5

### Example 53

### Ac-c[Lys-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl)

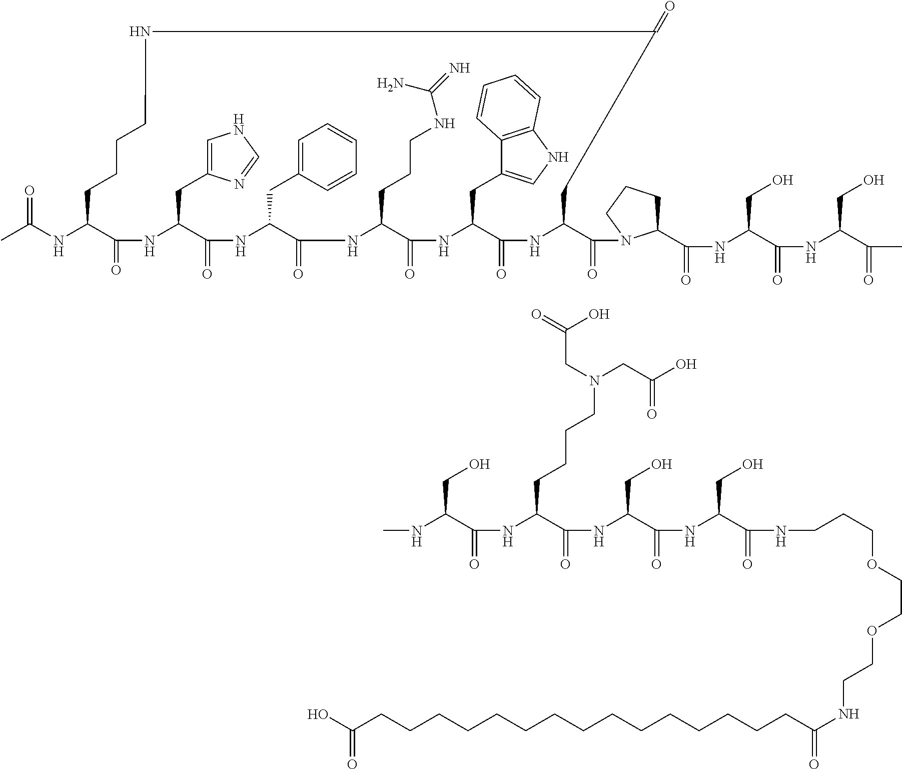

The compound can be prepared by the following synthetic route: preparation of resin Fmoc-Ser(tBu)-NH—(CH₂)₂—O—(CH₂)₂—O—(CH₂)₂—NH—CO—(CH₂)₁₆—CO—O-Wang-polystyrene as described for Example 3, then Fmoc-based synthesis of peptide resin Ac-Lys(Mtt)-His(Trt)-D-Phe-Arg(Pbf)-Trp(Boc)-Asp(2-phenylisopropyloxy)-Pro-Ser(tBu)-Ser(tBu)-Ser(tBu)-Lys(Boc)-Ser(tBu)-Ser(tBu)-NH—(CH₂)₂—O—(CH₂)₂—O—(CH₂)₂—NH—CO—(CH₂)₁₆—CO—O-Wang-polystyrene, then according to Example 9.

LCMS (system 1b): Rt=2.39 min; ((M+2)/2)=1058.5

### Example 54

### Ac-c[Glu-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂

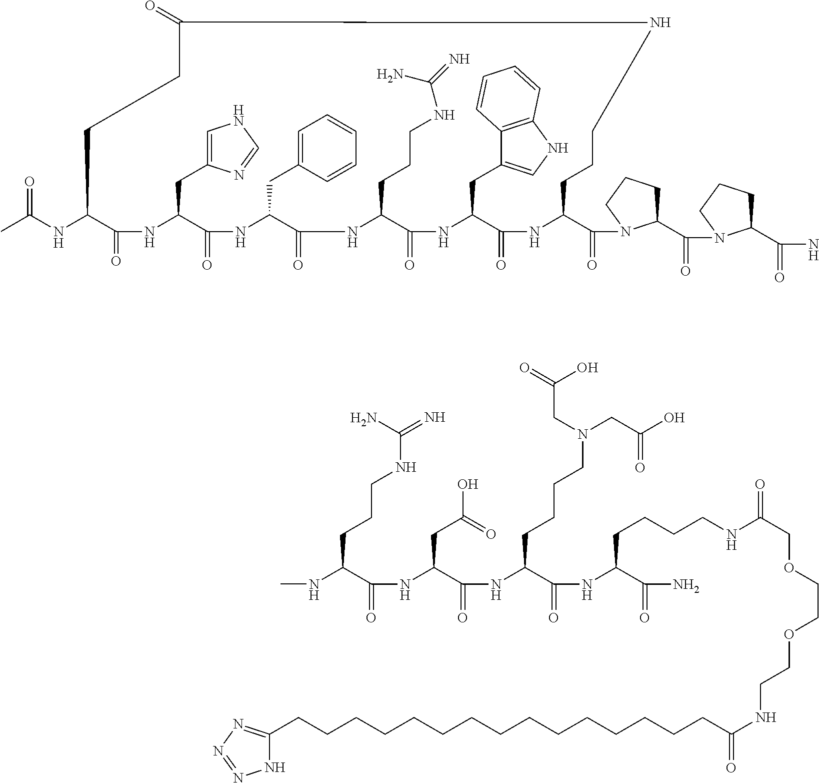

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Pro-Pro-Arg(Pbf)-Asp(OtBu)-Lys(Boc)-Lys(ivDde)-NH-RinkAM-polystyrene, then according to Example 6.

LCMS (system 1b): Rt=1.92 min; ((M+2)/2)=1101.0

### Example 55

### Ac-c[Orn-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂

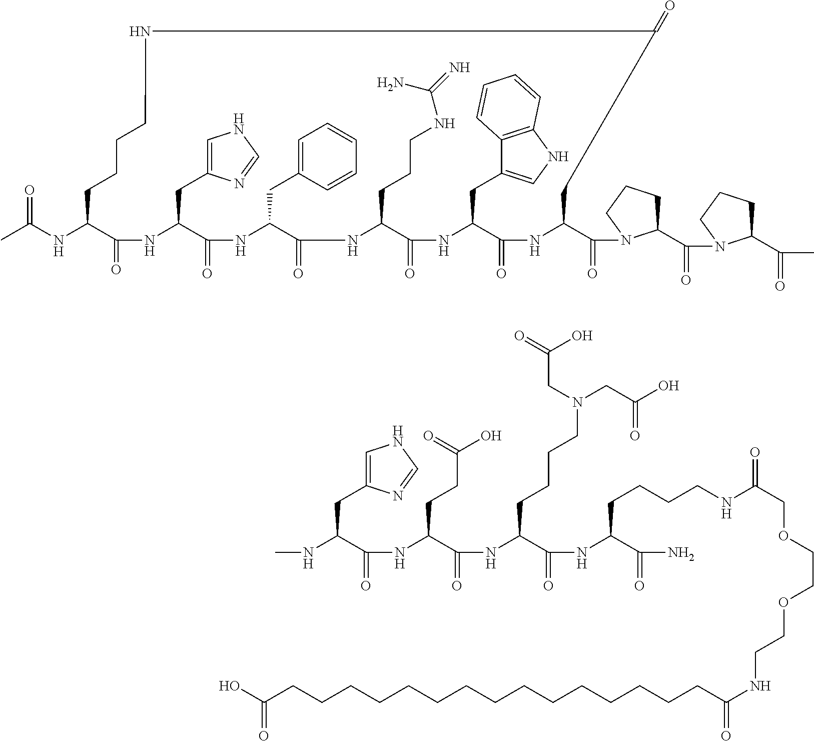

The compound can be prepared by the following synthetic route: Fmoc-based synthesis of peptide resin Ac-Orn(Mtt)-His(Trt)-D-Phe-Arg(Pbf)-Trp(Boc)-Asp(2-phenylisopropyloxy)-Pro-Pro-His(Trt)-Glu(OtBu)-Lys(Boc)-Lys(ivDde)-NH-RinkAM-polystyrene, then according to Example 7.

LCMS (system 1b): Rt=2.08 min; ((M+2)/2)=1080

### Example 56

### Ac-c[Glu-His-D-Phe-Arg-Trp-Orn]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂

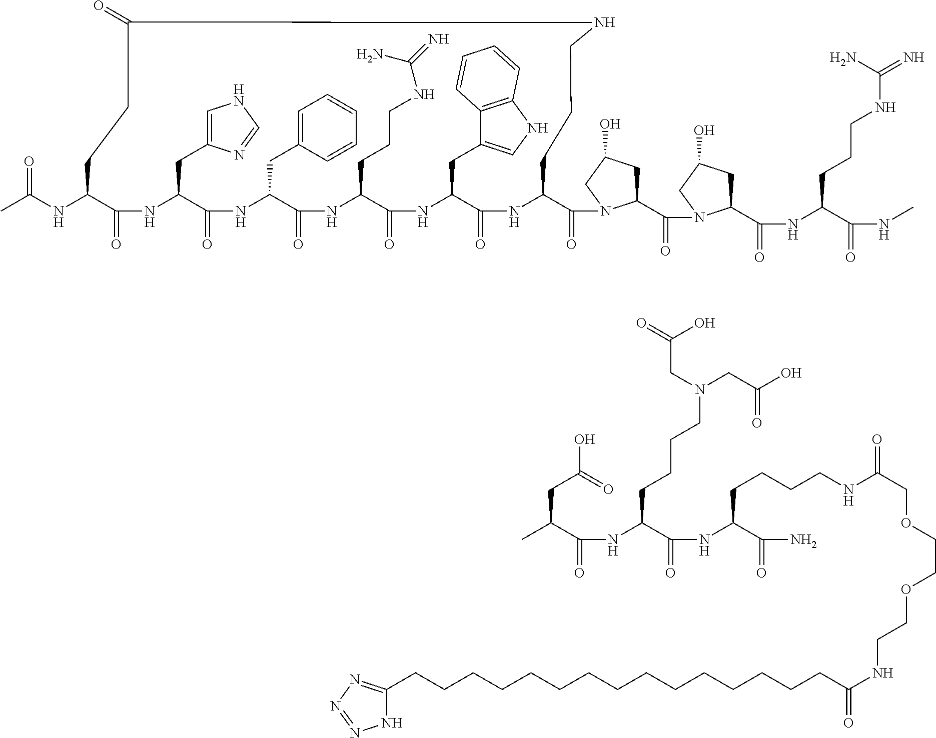 [SMILES: CNC(=O)[C@H](CCCNC(=N)N)NC(=O)[C@@H]1C[C@@H](O)CN1C(=O)[C@@H]1C[C@@H](O)CN1C(=O)[C@@H]1CCCNC(=O)CC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Glu(2-phenylisopropyloxy)-His(Trt)-D-Phe-Arg(Pbf)-Trp(Boc)-Orn(Mtt)-Hyp(tBu)-Hyp(tBu)-Arg(Pbf)-Asp(OtBu)-Lys(Boc)-Lys(ivDde)-NH-RinkAM-polystyrene, then according to Example 6.

LCMS (system 2): Rt=3.33 min; ((M+2)/2)=1117.0

### Example 57

### Ac-c[Orn-His-D-Phe-Arg-Trp-Glu]-Pro-Ser-Ser-Ser-Lys(biscarboxymethyl)-Ser-Ser-Lys({2-[2-(16-carboxyhexadecanoylamino)ethoxy]ethoxy}acetyl)-NH₂

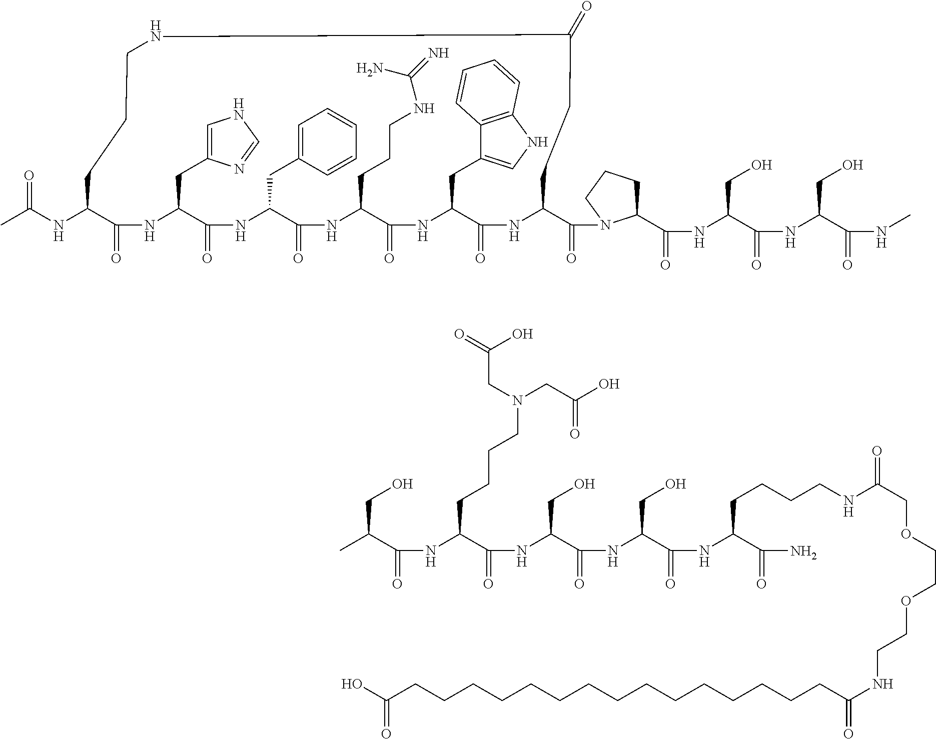

The compound can be prepared by the following synthetic route: Fmoc-based synthesis of peptide resin Ac-Orn(Mtt)-His(Trt)-D-Phe-Arg(Pbf)-Trp(Boc)-Glu(2-phenylisopropyloxy)-Pro-Ser(tBu)-Ser(tBu)-Ser(tBu)-Lys(Boc)-Ser(tBu)-Ser(tBu)-Lys(ivDde)-NH-RinkAM-polystyrene, then according to Example 7.

LCMS (system 3): Rt=3.71 min; ((M+2)/2)=1123.2

### Example 58

### Ac-c[homoCys-Ser-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂

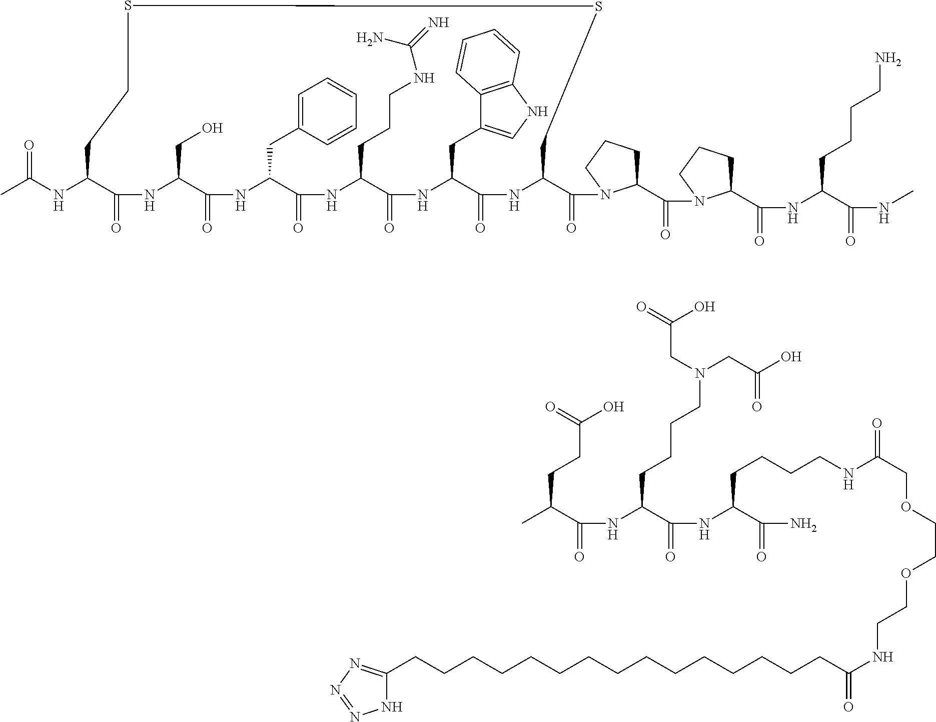 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](CO)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-Ser(tBu)-D-Phe-Arg(Pbf)-Trp(Boc)-Cys(Trt)-Pro-Pro-Lys(Boc)-Glu(OtBu)-Lys(ivDde)-Lys(Mtt)-NH-RinkAM-polystyrene, then selective removal of Mtt according to Procedure 3, acylation with Fmoc-8-amino-3,6-dioxaoctanoic acid+HOBt/DIC, deprotection with piperidine in NMP, acylation with 16-(tetrazol-5-yl)hexadecanoic acid+HOBt/DIC, removal of ivDde according to Procedure 1, solid-phase reductive alkylation with glyoxalic acid according to Procedure 5, cleavage from the resin and side-chain deprotection according to Procedure 7, disulfide cyclisation in solution according to Procedure 9, HPLC purification and freeze-drying.

LCMS (system 1b): Rt=2.05 min; ((M+2)/2)=1065.3

### Example 59

### Ac-c[Cys-Glu-His-D-Phe-Arg-Trp-Gly-Cys]-Pro-Pro-Lys-Glu-Lys(biscarboxymethyl)-Lys({2-[2-(15-carboxypentadecanoylamino)ethoxy]ethoxy}acetyl)-NH₂

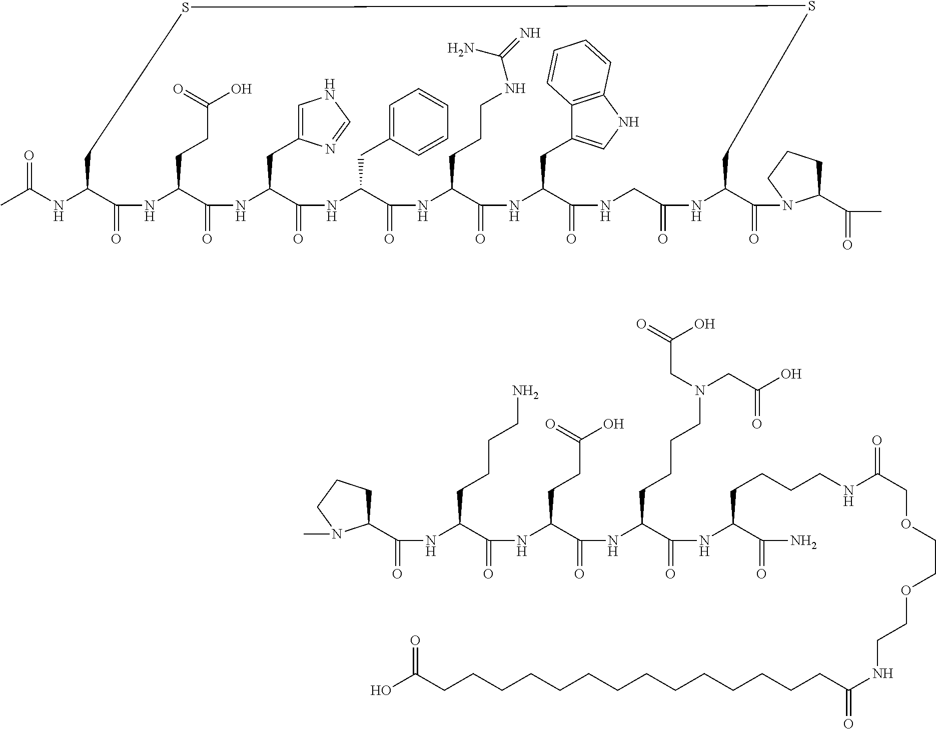

The compound can be prepared by the following synthetic route: Fmoc-based synthesis of peptide resin Ac-Cys(Trt)-Glu(OtBu)-His(Trt)-D-Phe-Arg(Pbf)-Trp(Boc)-Gly-Cys(Trt)-Pro-Pro-Lys(Boc)-Glu(OtBu)-Lys(ivDde)-Lys(Mtt)-NH-RinkAM-polystyrene, then selective removal of Mtt according to Procedure 3, acylation with Fmoc-8-amino-3,6-dioxaoctanoic acid+HOBt/DIC, deprotection with piperidine in NMP, acylation with hexadecanedioic acid monotert-butyl ester+HOBt/DIC, removal of ivDde according to 1, solid-phase reductive alkylation with glyoxalic acid according to Procedure 5, solid-phase disulfide cyclisation according to Procedure 10, cleavage from the resin and side-chain deprotection according to Procedure 8, HPLC purification and freeze-drying.

LCMS (system 1b): Rt=1.92 min; ((M+3)/3)=772.0

### Example 60

### Ac-c[homoCys-His-D-Phe-Arg-Trp-Cys]-Ser-Pro-Lys-Glu-Lys(biscarboxymethyl)-Lys({2-[2-(15-carboxypentadecanoylamino)ethoxy]ethoxy}acetyl)-NH₂

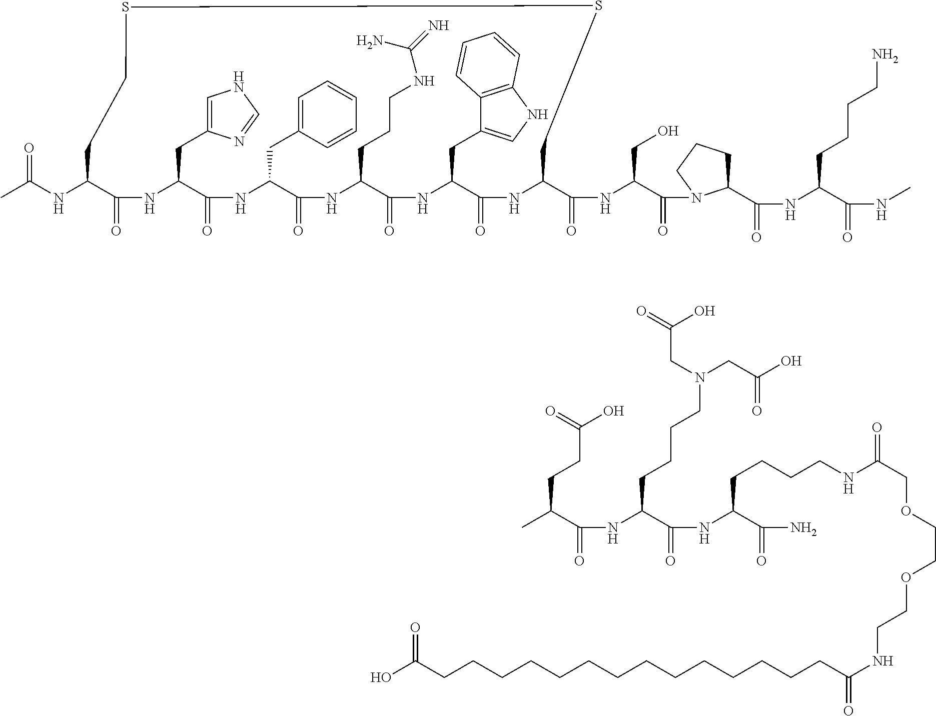 [SMILES: CNC(=O)[C@H](CCCCN)NC(=O)[C@@H]1CCCN1C(=O)[C@H](CO)NC(=O)[C@@H]1CSSCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Ser(tBu)-Pro-Lys(Boc)-Glu(OtBu)-Lys(ivDde)-Lys(Mtt)-NH-RinkAM-polystyrene, then according to Example 59.

LCMS (system 1b): Rt=1.92 min; ((M+2)/2)=1066.5

### Example 61

### Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

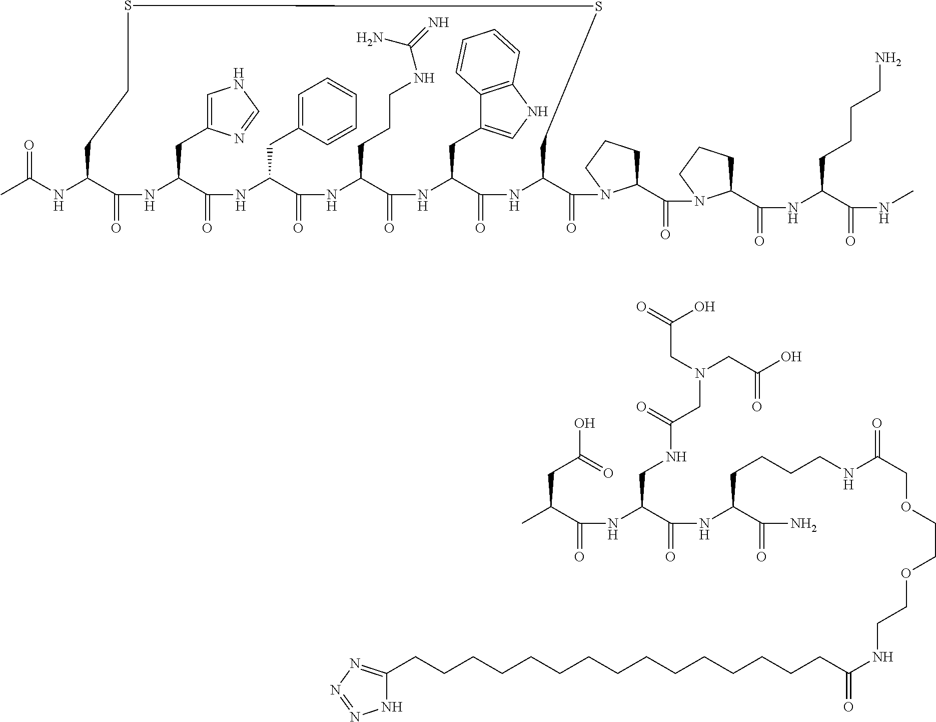 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Pro-Pro-Lys(Boc)-Asp(OtBu)-Dap(Mtt)-Lys(ivDde)-NH-RinkAM-polystyrene, then selective removal of Mtt according to Procedure 3, acylation with bis(tert-butoxycarbonylmethyl)aminoacetic acid+PyBOP/DIPEA according to Procedure 6, removal of ivDde according to Procedure 1, acylation with Fmoc-8-amino-3,6-dioxaoctanoic acid+HOBt/DIC, deprotection with piperidine in NMP, acylation with 16-(tetrazol-5-yl)hexadecanoic acid+HOBt/DIC, cleavage from the resin and side-chain deprotection according to Procedure 7, disulfide cyclisation in solution according to Procedure 9, HPLC purification and freeze-drying.

LCMS (system 3): Rt=3.72 min; ((M+2)/2)=1091.1

### Example 62

### Ac-c[homoCys-His-D-Phe-Arg-Trp-Cys]-Ser-Pro-His-Glu-Lys(biscarboxymethyl)-Lys({2-[2-(17-carboxyheptadecanoylamino)ethoxy]ethoxy}acetyl)-NH₂

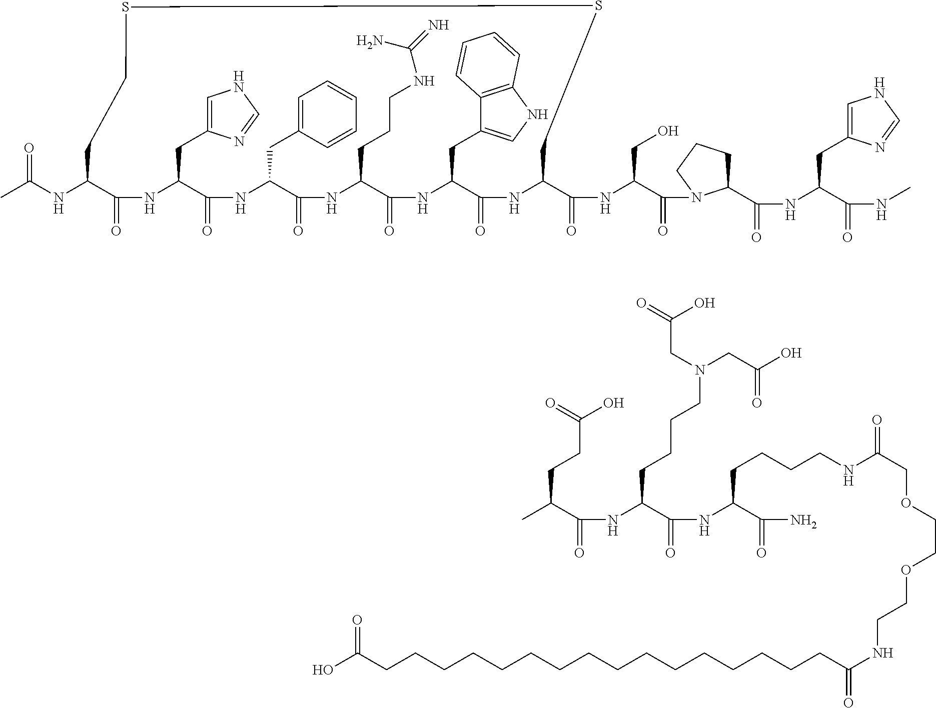 [SMILES: CNC(=O)[C@H](Cc1c[nH]cn1)NC(=O)[C@@H]1CCCN1C(=O)[C@H](CO)NC(=O)[C@@H]1CSSCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Ser(tBu)-Pro-His(Trt)-Glu(OtBu)-Lys(ivDde)-Lys(Mtt)-NH-RinkAM-polystyrene, then selective removal of Mtt according to Procedure 3, acylation with Fmoc-8-amino-3,6-dioxaoctanoic acid+HOBt/DIC, deprotection with piperidine in NMP, acylation with octadecanedioic acid monotert-butyl ester+HOBt/DIC, removal of ivDde according to Procedure 1, solid-phase reductive alkylation with glyoxalic acid according to Procedure 5, solid-phase disulfide cyclisation according to Procedure 10, cleavage from the resin and side-chain deprotection according to Procedure 8, HPLC purification and freeze-drying.

LCMS (system 1b): Rt=2.10 min; ((M+2)/2)=1085.0

### Example 63

### Ac-c[Cys-Glu-His-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂

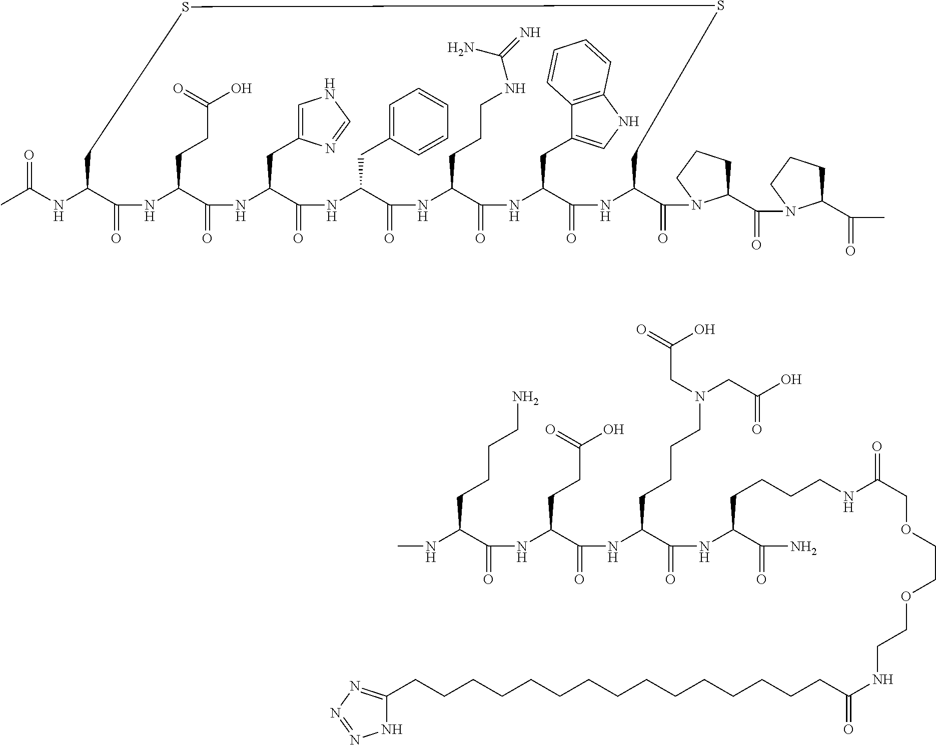 [SMILES: CC(=O)N[C@H]1CSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@H](CCC(=O)O)NC1=O ; CN[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-Cys(Trt)-Glu(OtBu)-His(Trt)-D-Phe-Arg(Pbf)-Trp(Boc)-Cys(Trt)-Pro-Pro-Lys(Boc)-Glu(OtBu)-Lys(ivDde)-Lys(Mtt)-NH-RinkAM-polystyrene, then according to Example 58.

LCMS (system 1a): Rt=1.87 min; ((M+2)/2)=1148.0

### Example 64

### Ac-c[homoCys-His-D-Phe-Arg-Trp-Cys]-Pro-Pro-Lys-Glu-Dap(BCMA)-Lys[(2-{2-[16-(tetrazol-5-yl)hexadecanoylamino]ethoxy}ethoxy)acetyl]-NH₂

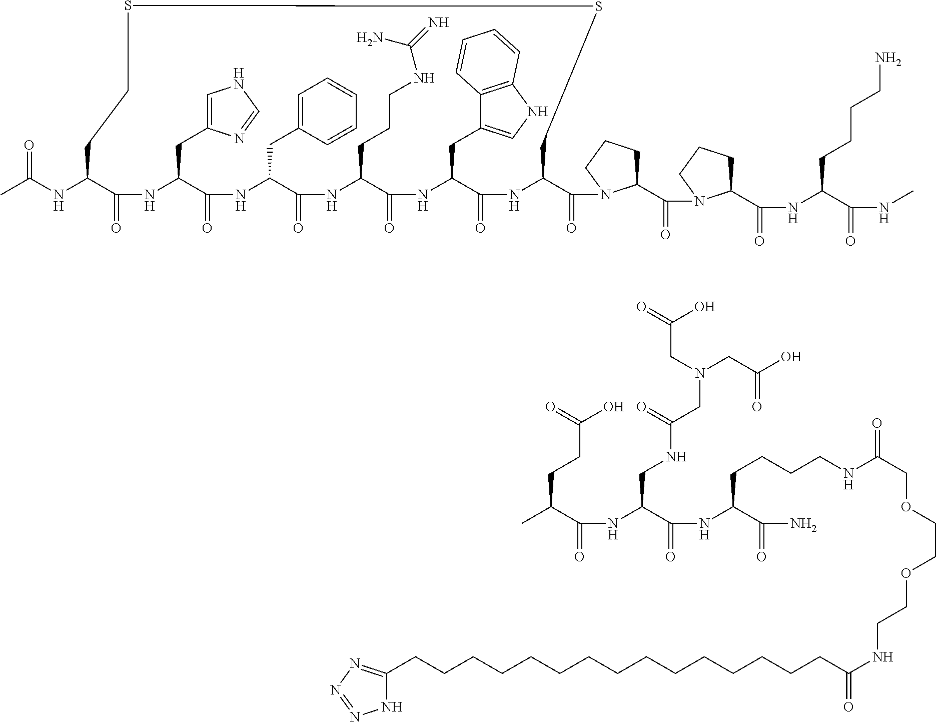 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CCC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Pro-Pro-Lys(Boc)-Glu(OtBu)-Dap(Mtt)-Lys(ivDde)-NH-RinkAM-polystyrene, then according to Example 61.

MALDI-MS: m/z=2197

### Example 65

### Ac-c[homoCys-His-D-Phe-Arg-Trp-Cys]-Pro-Pro-Lys-Asp-Lys(BCMA)-Lys[(2-{2-[16-(tetrazol-5-yl)hexadecanoylamino]ethoxy}ethoxy)acetyl]-NH₂

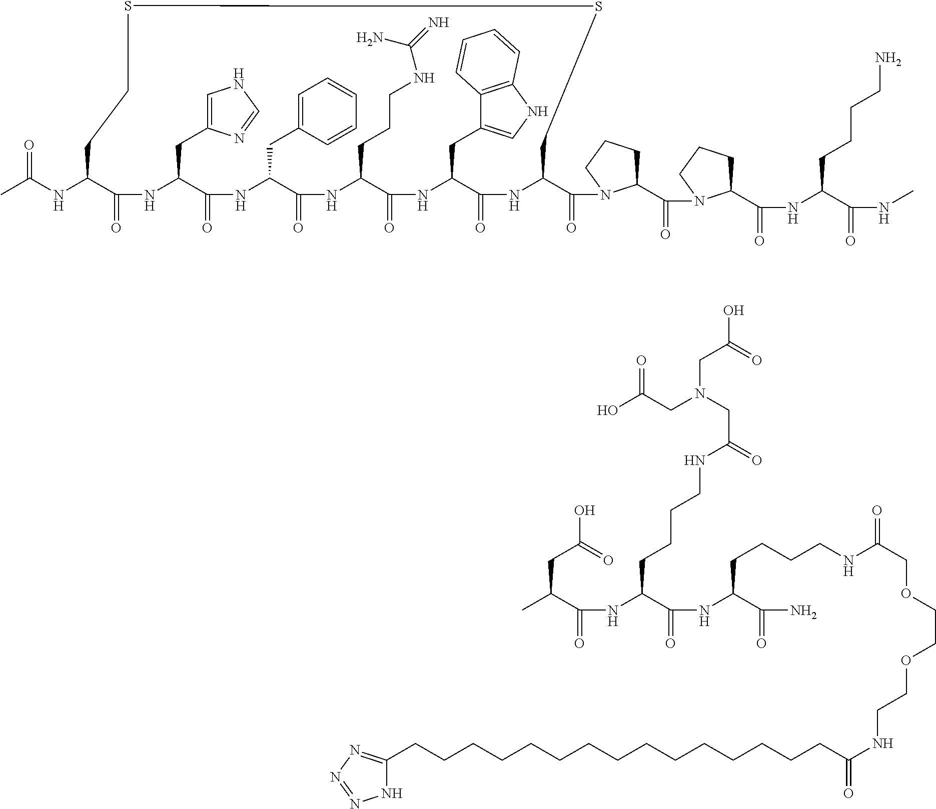 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Pro-Pro-Lys(Boc)-Asp(OtBu)-Lys(Mtt)-Lys(ivDde)-NH-RinkAM-polystyrene, then according to Example 61.

MALDI-MS: m/z=2224

### Example 66

### Ac-c[homoCys-Gln-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

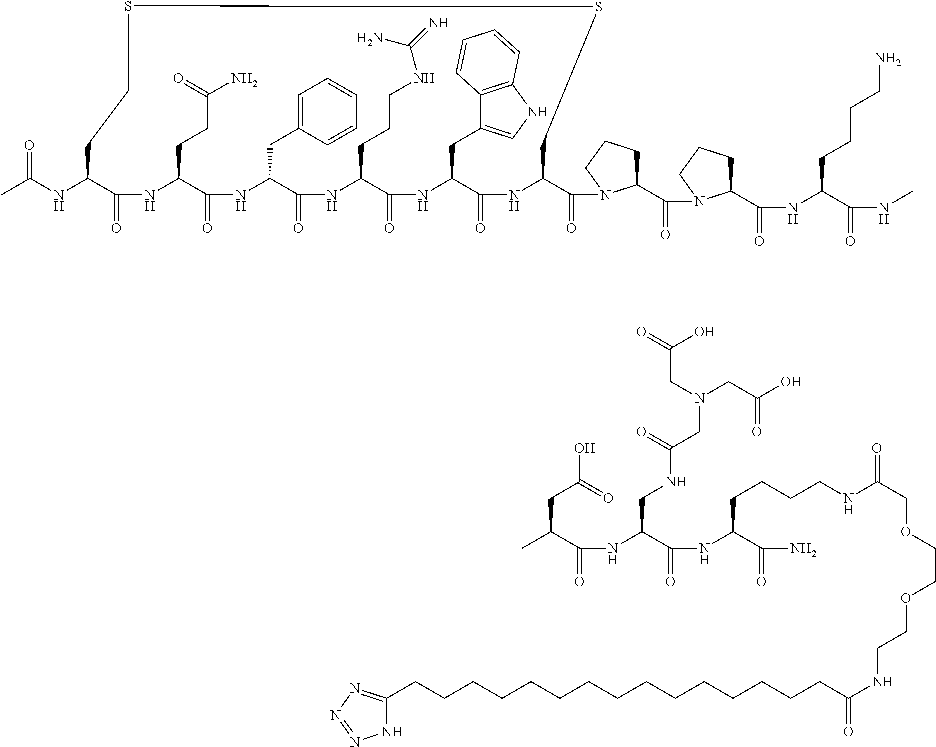 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N[C@@H](CCC(N)=O)C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-Gln(Trt)-D-Phe-Arg(Pbf)-Trp(Boc)-Cys(Trt)-Pro-Pro-Lys(Boc)-Asp(OtBu)-Dap(Mtt)-Lys(ivDde)-NH-RinkAM-polystyrene, then according to Example 61.

LCMS (system 1c): Rt=2.01 min; ((M+2)/2)=1086.7

### Example 67

### Ac-c[homoCys-Hyp-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂

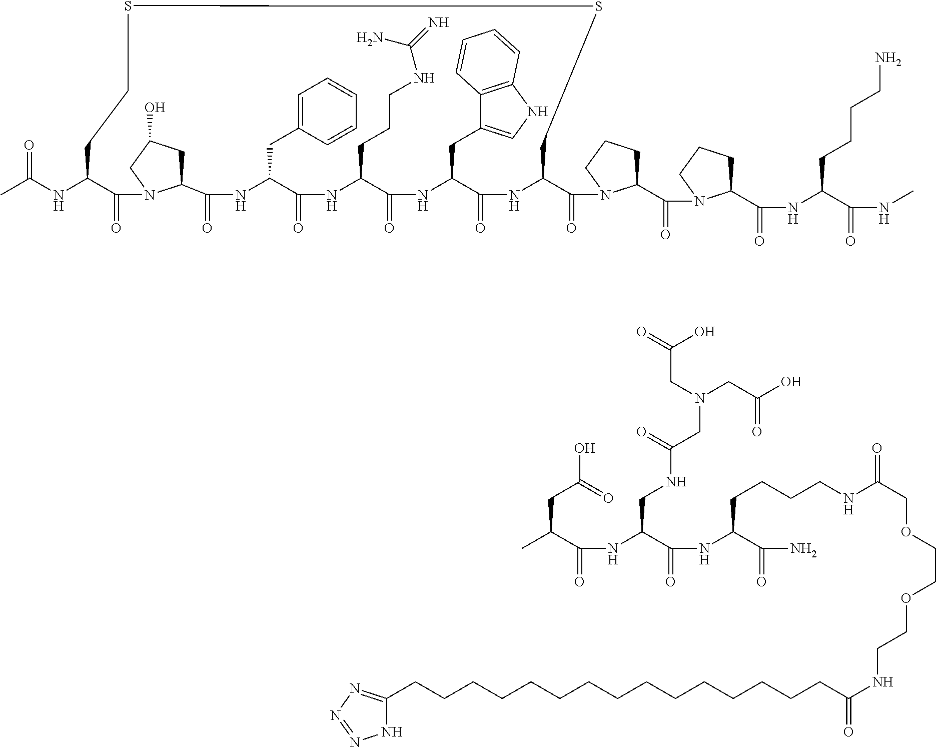 [SMILES: CNC(=O)[C@H](CCCCN)NC(=O)[C@@H]1CCCN1C(=O)[C@@H]1CCCN1C(=O)[C@@H]1CSSCC[C@H](NC(C)=O)C(=O)N2C[C@H](O)C[C@H]2C(=O)N[C@H](Cc2ccccc2)C(=O)N[C@@H](CCCNC(=N)N)C(=O)N[C@@H](Cc2c[nH]c3ccccc23)C(=O)N1 ; C[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]

The compound can be prepared by the following synthetic route: Fmoc-based synthesis of peptide resin Ac-homoCys(Trt)-Hyp(tBu)-D-Phe-Arg(Pbf)-Trp(Boc)-Cys(Trt)-Pro-Pro-Lys(Boc)-Asp(OtBu)-Dap(Mtt)-Lys(ivDde)-NH-RinkAM-polystyrene, then according to Example 61.

MALDI-MS: m/z=2154

### Example 68

### Ac-c[Cys-Glu-His-D-Phe-Arg-Trp-Gly-Cys]-Pro-Pro-Lys-Glu-Glu-Lys({2-[2-(15-carboxypentadecanoylamino)ethoxy]ethoxy}acetyl)-NH₂

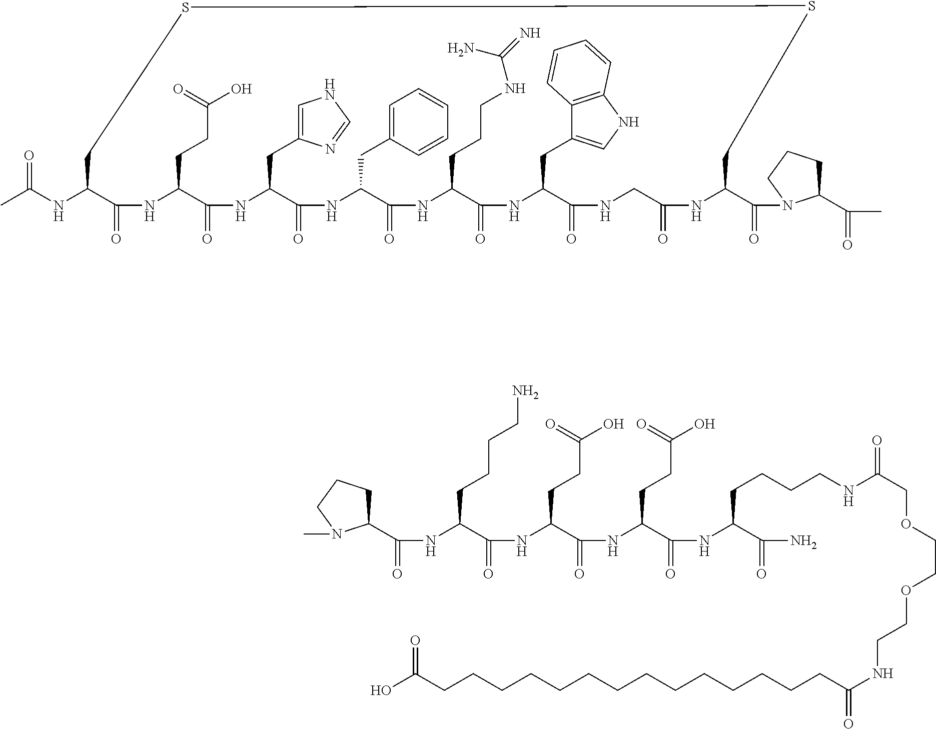

The compound can be prepared by the following synthetic route: Fmoc-based synthesis of peptide resin Ac-Cys(Trt)-Glu(OtBu)-His(Trt)-D-Phe-Arg(Pbf)-Trp(Boc)-Gly-Cys(Trt)-Pro-Pro-Lys(Boc)-Glu(OtBu)-Glu(OtBu)-Lys(Mtt)-NH-RinkAM-polystyrene, then selective removal of Mtt according to Procedure 3, acylation with Fmoc-8-amino-3,6-dioxaoctanoic acid+HOBt/DIC, deprotection with piperidine in NMP, acylation with hexadecanedioic acid monotert-butyl ester+HOBt/DIC, solid-phase disulfide cyclisation according to Procedure 10, cleavage from the resin and side-chain deprotection according to Procedure 8, HPLC purification and freeze-drying.

LCMS (system 1b): Rt=1.75 min; ((M+2)/2)=1100.0

### Example 69

### Ac-c[homoCys-His-D-Phe-Arg-Trp-Cys]-Pro-Pro-Lys-Asp-Dap(BCMA)-Lys({2-[2-(13-carboxytridecanoylamino)ethoxy]ethoxy}acetyl)-NH₂

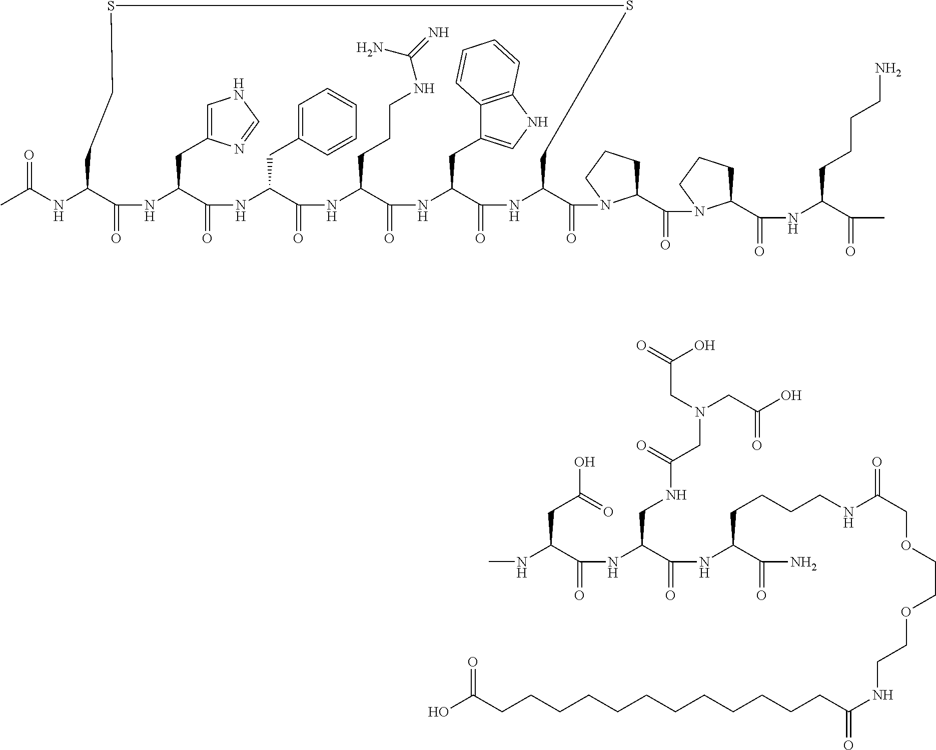 [SMILES: CC(=O)N[C@H]1CCSSC[C@@H](C(=O)N2CCC[C@H]2C(=O)N2CCC[C@H]2C(=O)N[C@@H](CCCCN)C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCC(=O)O)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Pro-Pro-Lys(Boc)-Asp(OtBu)-Dap(Mtt)-Lys(ivDde)-NH-RinkAM-polystyrene, then selective removal of Mtt according to Procedure 3, acylation with bis(tert-butoxycarbonylmethyl)aminoacetic acid+PyBOP/DIPEA according to Procedure 6, removal of ivDde according to Procedure 1, acylation with Fmoc-8-amino-3,6-dioxaoctanoic acid+HOBt/DIC, deprotection with piperidine in NMP, acylation with tetradecanedioic acid mono-tert-butyl ester (available by the synthetic procedure described in: U. Widmer, Synthesis 1983, 135)+HOBt/DIC, solid-phase disulfide cyclisation according to Procedure 10, cleavage from the resin and side-chain deprotection according to Procedure 8, HPLC purification and freeze-drying.

MALDI-MS: m/z=2114

### Example 70

### Ac-c[homoCys-His-D-Phe-Arg-Trp-Cys]-Pro-Pro-Ser-Glu-Lys(biscarboxymethyl)-Lys({2-[2-(15-carboxypentadecanoylamino)ethoxy]ethoxy}acetyl)-NH₂

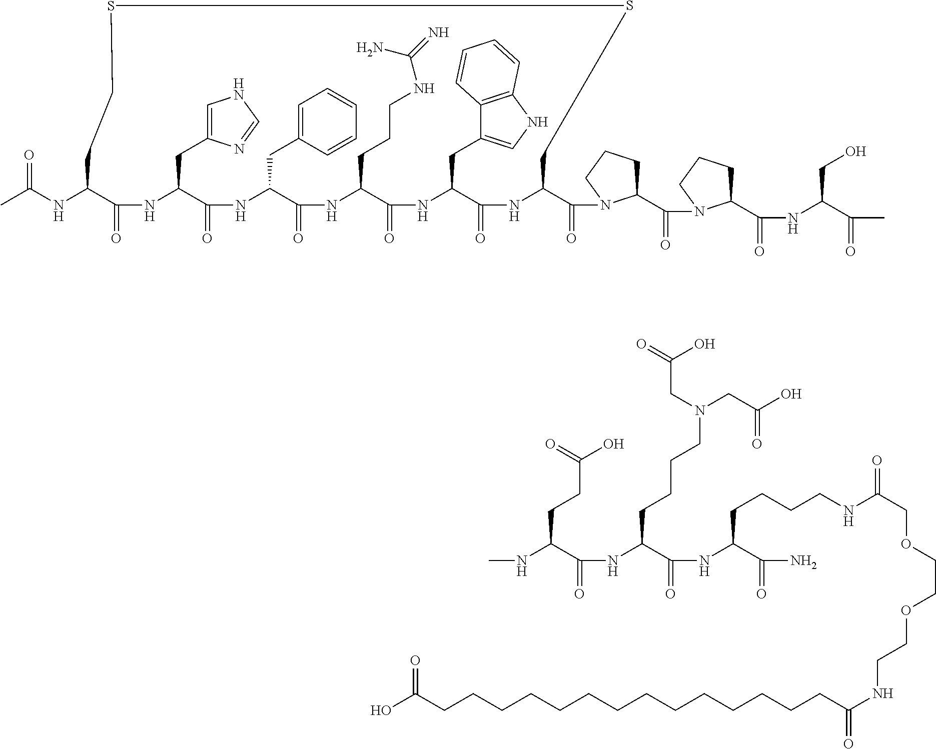 [SMILES: CC(=O)N[C@H]1CCSSC[C@@H](C(=O)N2CCC[C@H]2C(=O)N2CCC[C@H]2C(=O)N[C@@H](CO)C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

The compound can be prepared by the following synthetic route: Fmoc-based synthesis of peptide resin Ac-homoCys(Trt)-His(Trt)-D-Phe-Arg(Pbf)-Trp(Boc)-Cys(Trt)-Pro-Pro-Ser(tBu)-Glu(OtBu)-Lys(ivDde)-Lys(Mtt)-NH-RinkAM-polystyrene, then according to Example 59.

LCMS (system 1b): Rt=2.06 min; ((M+2)/2)=1051.0

### Example 71

### Preparation of GLP-1 derivatives

The following GLP-1 compounds were prepared (all being derivatives of analogues of GLP-1(7-37)):

### Compound G1:

N-epsilon26-((S)-4-Carboxy-4-hexadecanoylamino-butyryl)[Arg34]GLP-1-(7-37), which may also be designated Arg³⁴Lys²⁶(Nε-(μ-glutamyl(Nα-hexadecanoyl)))-GLP-1(7-37)-OH:

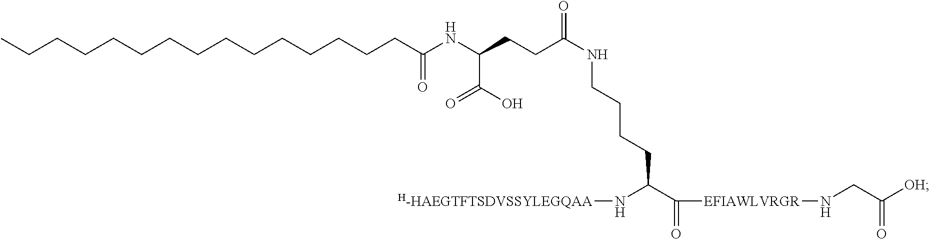

### Compound G2:

N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34,Lys37]GLP-1-(7-37):

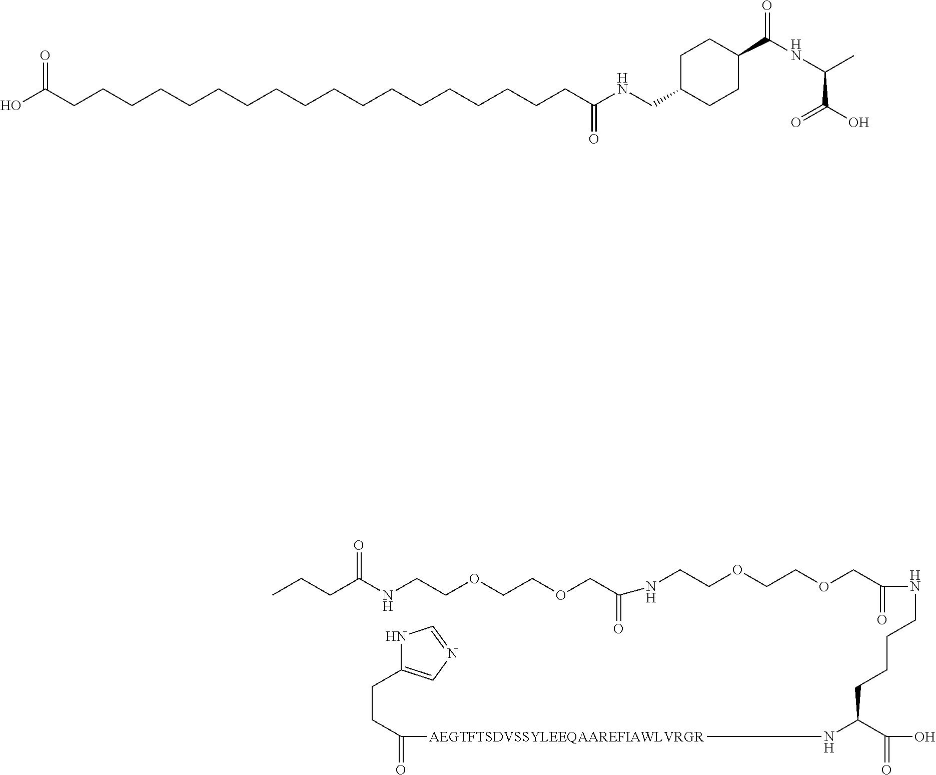

### Compound G3:

N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib8,Arg34]G LP-1-(7-37)

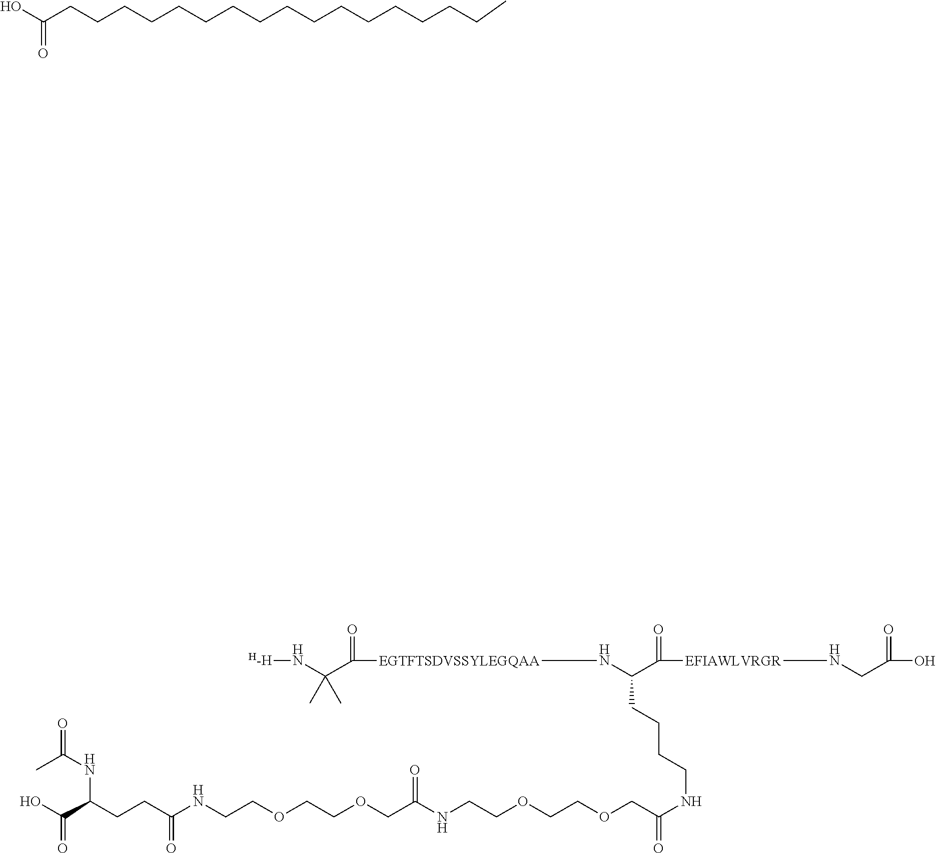

### Compound G4:

N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35,Lys37]GLP-1-(7-37)

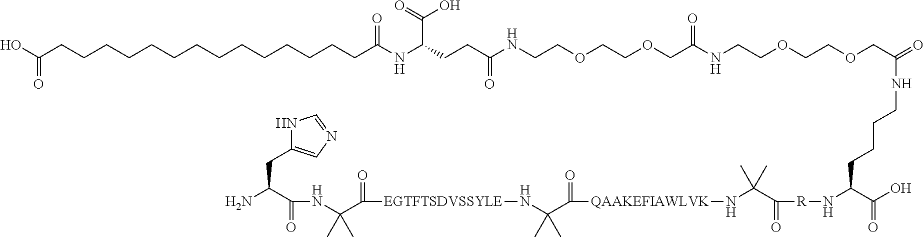

Compound G1 was prepared as described in Example 37 of WO 98/08871. Compound G2 was prepared as described in Example 26 of WO 09030771. Compound G3 was prepared as described in Example 4 of WO 2006/097537.

Novel compound G4 was prepared in similar fashion to the methods described in WO 09/030,771, using a CEM Liberty peptide synthesizer.

LCMS: m/z=1046 (M/4)

Calculated (M)=4184.8

LCMS was performed on a setup consisting of Waters Acquity HPLC system and LCT Premier XE mass spectrometer from Micromass. The HPLC pump was connected to two eluent reservoirs containing:

A: 0.1% Formic acid in water
B: 0.1% Formic acid in acetonitrile

The analysis was performed at room temperature (RT) by injecting an appropriate volume of the sample (preferably 2-100) onto the column which was eluted with a gradient of A and B. The HPLC conditions, detector settings and mass spectrometer settings used are given in the following table:

Column: Waters Acquity HPLC BEH, C-18, 1.7 μm, 2.1 mm×50 mm
Gradient: 5%-95% acetonitrile linear during 4.0 min at 0.4 ml/min
Detection: 214 nm (analogue output from DAD (Diode Array Detector))
MS ionisation mode: API-ES (atmospheric pressure ionisation electrospray), Scan 100-2000 amu (atomic mass units), step 0.1 amu

### Solubility Data

### Solubility Data of Compounds

From a stock solution in vehicle, either H₂O or 10 mM phosphate, containing 1 mg (as weighed) compound per ml, 8-11 aliquots are withdrawn and pH-adjusted individually with HCl/NaOH, to cover the whole pH range. After incubation at room temperature for 1-3 days, the samples were centrifuged at 20.000 g for 20 min., the pH was measured and the solubility determined by quantification of content in the supernatant by UV-detection (e_{(280 nm)}=5500M⁻¹ cm⁻¹).

TABLE 1
Solubility data

| Ex- | | | | | |
|---|---|---|---|---|---|
| am- | | | Solubility | | |
| ple | | | | Conc. | Vehi- |
| nr. | Compound | Molecule | pH | (μM) | cle |
| 2 | Ac-c[Asp- His-D- Phe-Arg- Trp-Orn]- Pro-Pro- Arg-Asp- Lys(bis- carboxy- methyl)- Lys[(2-{2- [16- (tetrazol-5- yl) hexadeca- noyl- amino] ethoxy} ethoxy) acetyl]- NH2 | 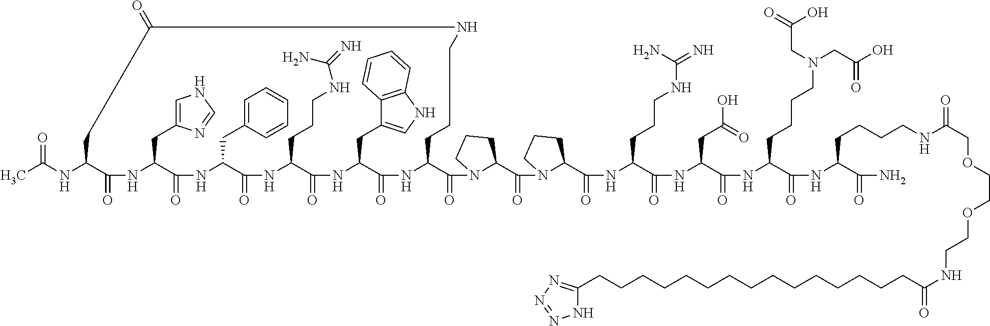 [SMILES: CC(=O)N[C@H]1CC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.6 7.6 7.4 7.3 7.2 7.0 6.4 5.8 | 458 454 482 465 267 172 67 26 21 | 10 mM phos- phate |
| 3 | Ac-c[Dab- His-D- Phe-Arg- Trp-Asp]- Pro-Ser- Ser-Ser- Lys(bis- carboxy- methyl)- Ser-Ser- NH(2-{2- [2-(17- carboxy- hepta- decanoyl- amino) ethoxy] ethoxy} ethyl) | 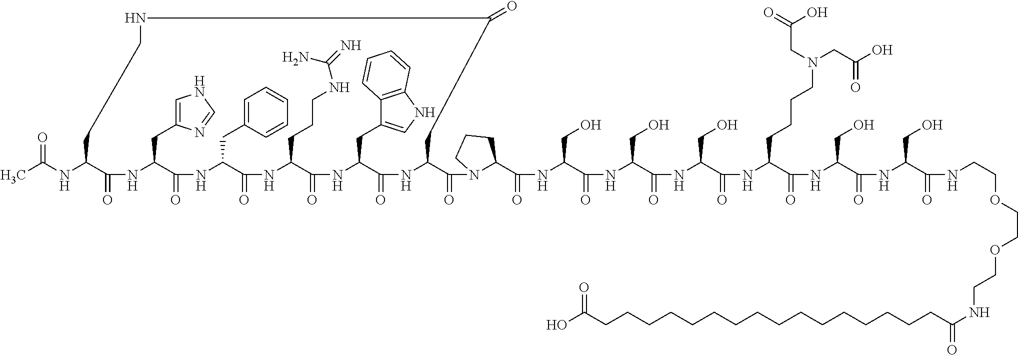 [SMILES: CC(=O)N[C@H]1CCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.7 7.4 7.2 7.1 6.9 6.7 6.1 5.6 4.9 | 360 345 396 401 396 295 154 54 32 24 | 10 mM phos- phate |
| 5 | Ac-c [homoCys- His-D- Phe-Arg- Trp-Cys]- Pro-Pro- Lys-Glu- Lys(bis- carboxy- methyl)- Lys({2-[2- (15- carboxy- penta- decanoyl- amino) ethoxy] ethoxy} acetyl)- NH2 | 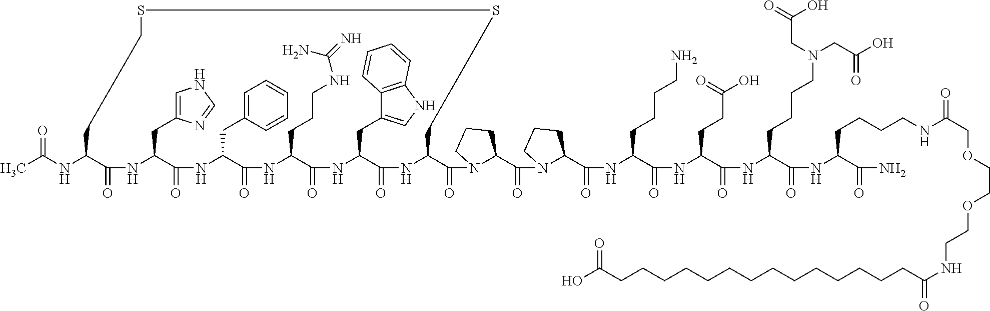 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.8 7.7 7.7 7.3 7.1 6.7 6.1 4.9 4.6 3.8 | 509 498 504 496 489 320 200 233 452 486 | water |
| 6 | Ac-c[Glu- His-D- Phe-Arg- Trp-Dap]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 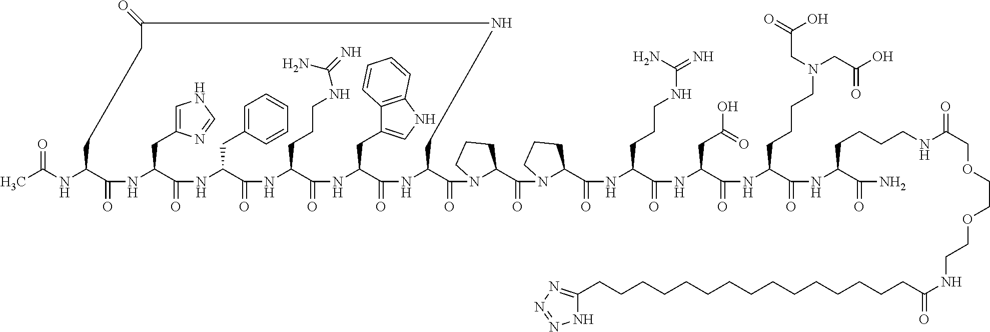 [SMILES: CC(=O)N[C@H]1CCC(=O)NC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.8 7.6 7.5 7.4 7.2 6.9 6.3 5.9 | 280 254 290 270 208 82 44 24 23 | 10 mM phos- phate |
| 7 | Ac-c[Lys- His-D- Phe-Arg- Trp-Glu]- Pro-Pro- His-Glu- Lys(bis- carboxy- methyl)- Lys({2-[2- (16- carboxy- hexa- decanoyl- amino) ethoxy] ethoxy} acetyl)- NH2 | 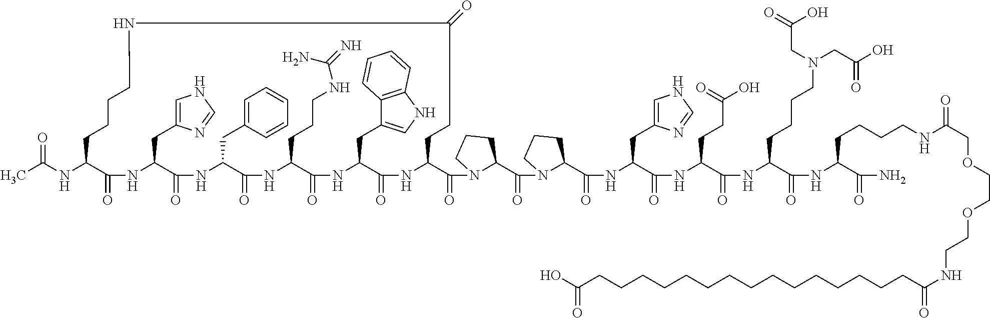 [SMILES: CC(=O)N[C@H]1CCCCNC(=O)CC[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.6 7.4 7.2 6.9 6.7 6.6 6.3 5.8 | 436 448 438 417 434 408 295 173 121 | 10 mM phos- phate |
| 8 | Ac-c[Orn- His-D- Phe-Arg- Trp-Glu]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 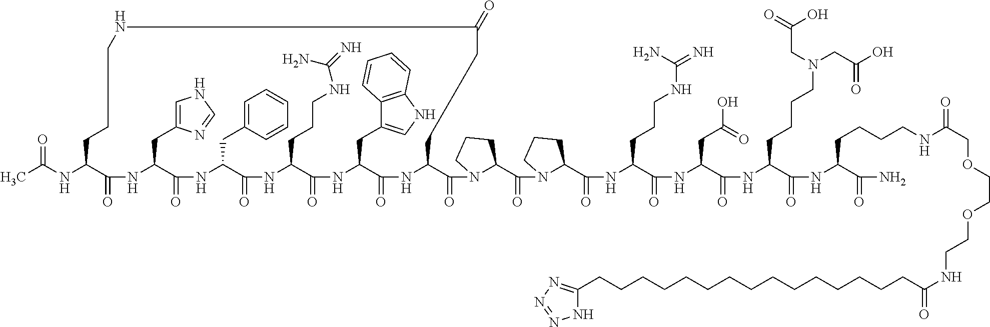 [SMILES: CC(=O)N[C@H]1CCCNC(=O)CC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.8 7.6 7.5 7.4 7.3 7.2 7.0 6.8 6.5 | 556 582 538 551 482 268 91 44 35 | 10 mM phos- phate |
| 9 | Ac-c[Dab- His-D- Phe-Arg- Trp-Asp]- Pro-Pro- Arg-Glu- Lys(bis- carboxy- methyl)- Ser-Ser- NH(2-{2- [2-(17- carboxy- hepta- decanoyl- amino) ethoxy] ethoxy} ethyl) | 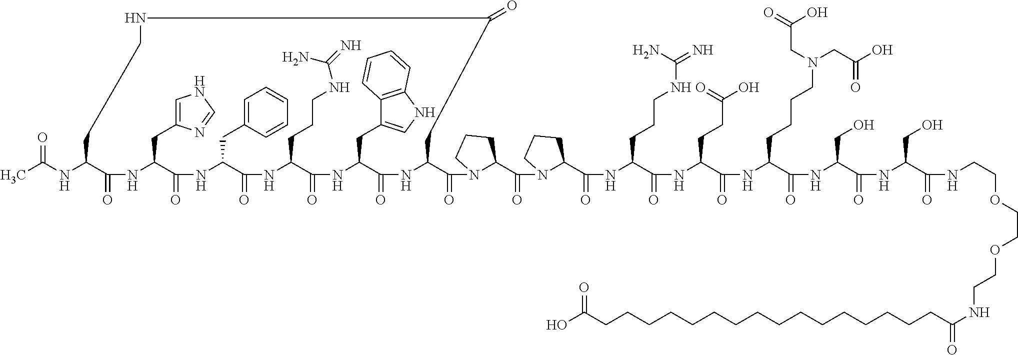 [SMILES: CC(=O)N[C@H]1CCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.2 7.9 7.7 7.4 7.3 6.6 5.3 5.2 4.7 | 280 259 242 183 158 79 40 22 26 | 10 mM phos- phate |
| 10 | Ac-c[Orn- His-D- Phe-Arg- Trp-Asp]- Pro-Pro- His-Glu- Lys(bis- carboxy- methyl)- Lys[(2-{2- [16- (tetrazol-5- yl)hexa- decanoyl- amino] ethoxy} ethoxy) acetyl]- NH2 | 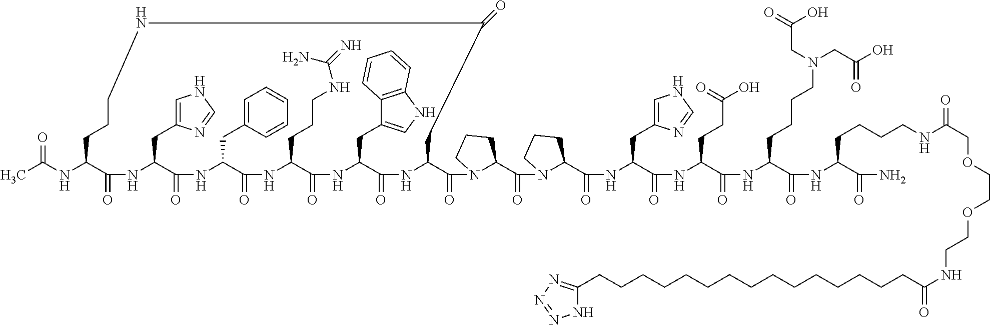 [SMILES: CC(=O)N[C@H]1CCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.7 7.4 7.3 7.0 6.7 6.6 6.4 6.0 | 531 521 516 517 517 514 287 88 35 | 10 mM phos- phate |
| 11 | Ac-c[Lys- His-D- Phe-Arg- Trp-Asp]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 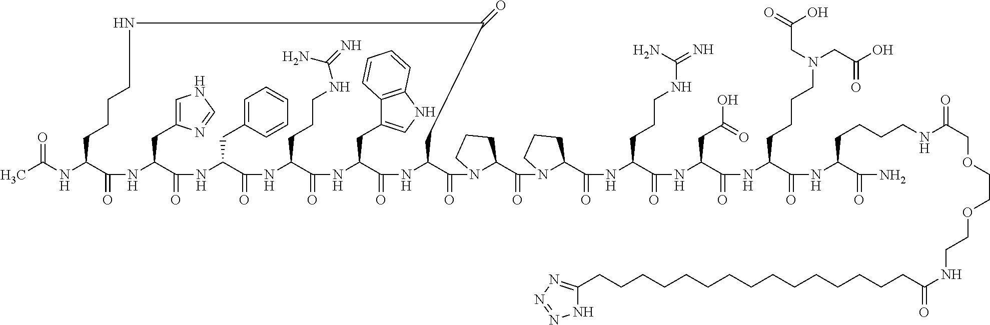 [SMILES: CC(=O)N[C@H]1CCCCNC(=O)C[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.8 7.6 7.3 7.2 7.0 6.4 6.0 5.3 4.8 | 479 472 479 364 147 36 25 29 51 | 10 mM phos- phate |
| 12 | Ac-c(Orn- His-D- Phe-Arg- Trp-Asp]- Pro-Ser- Ser-Ser- Lys(bis- carboxy- methyl)- Ser-Ser- Lys({2-[2- (15- carboxy- penta- decanoyl- amino) ethoxy] ethoxy} acetyl)- NH2 | 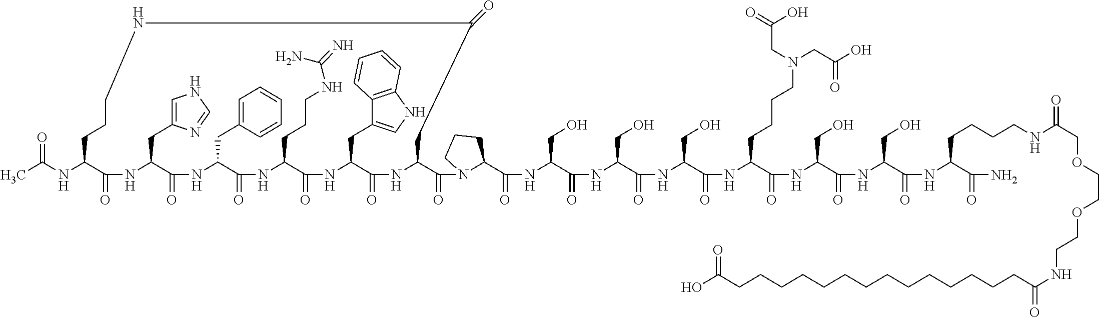 [SMILES: CC(=O)N[C@H]1CCCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.8 7.5 7.2 7.0 6.7 6.3 5.8 4.8 | 439 480 405 479 426 431 400 300 228 | 10 mM phos- phate |
| 13 | Ac-c[Glu- His-D- Phe-Arg- Trp-Orn]- Pro-Pro- Asp- Lys(bis- carboxy- methyl)- Lys({2-[2- (15- carboxy- penta- decanoyl- amino) ethoxy] ethoxy} acetyl)- NH2 | 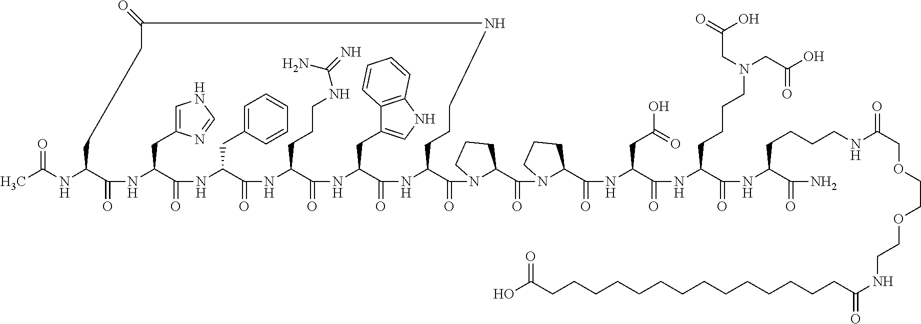 [SMILES: CC(=O)N[C@H]1CCC(=O)NCCC[C@@H](C(=O)N2CCC[C@H]2C(=O)N2CCC[C@H]2C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.3 7.1 6.8 6.5 6.1 5.7 5.5 | 493 472 467 506 459 469 473 494 348 | 10 mM phos- phate |
| 14 | Ac-c[Orn- His-D- Phe-Arg- Trp-Asp]- Pro-Ser- Ser-Ser- Lys(bis- carboxy- methyl)- Ser-Ser- Lys({2-[2- (17- carboxy- hepta- decanoyl- amino) ethoxy] ethoxy} acetyl)- NH2 | 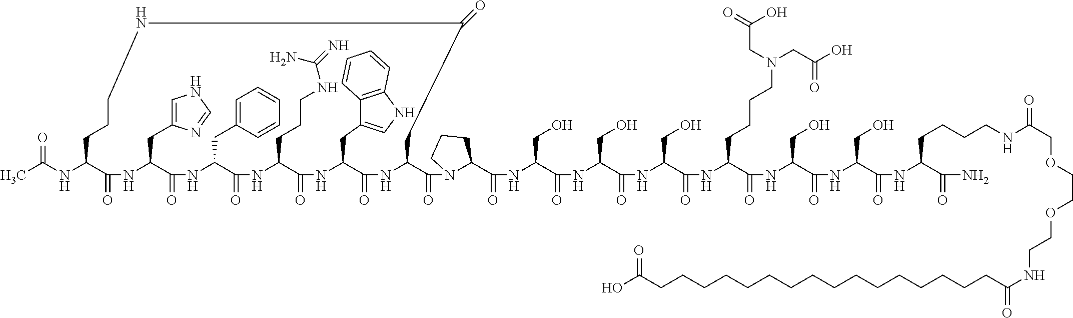 [SMILES: CC(=O)N[C@H]1CCCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.4 7.3 7.2 7.0 6.9 6.7 6.4 | 517 506 513 535 507 485 403 148 61 | 10 mM phos- phate |
| 15 | Ac-c[Dab- His-D- Phe-Arg- Trp-Asp]- Pro-Pro- Arg-Glu- Lys(bis- carboxy- methyl)- Lys[(2-{2- [16- (tetrazol-5- yl)hexa- decanoyl- amino] ethoxy} ethoxy) acetyl]- NH2 | 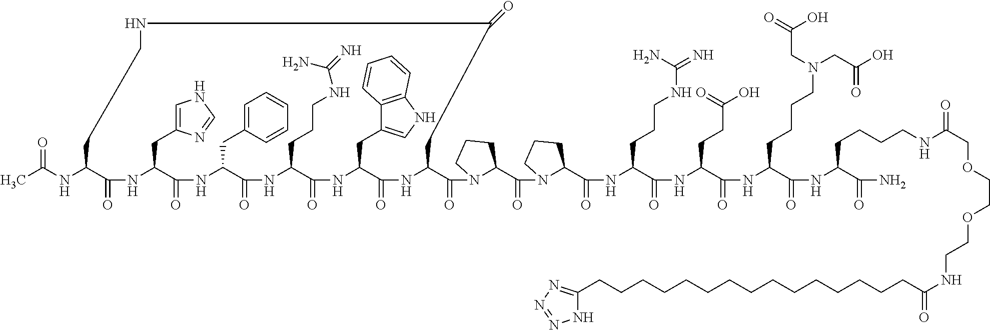 [SMILES: CC(=O)N[C@H]1CCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.7 7.4 7.3 7.1 6.8 6.1 5.0 | 417 441 461 445 395 147 60 26 49 | 10 mM phos- phate |
| 16 | Ac-c[Glu- His-D- Phe-Arg- Trp-Dab]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 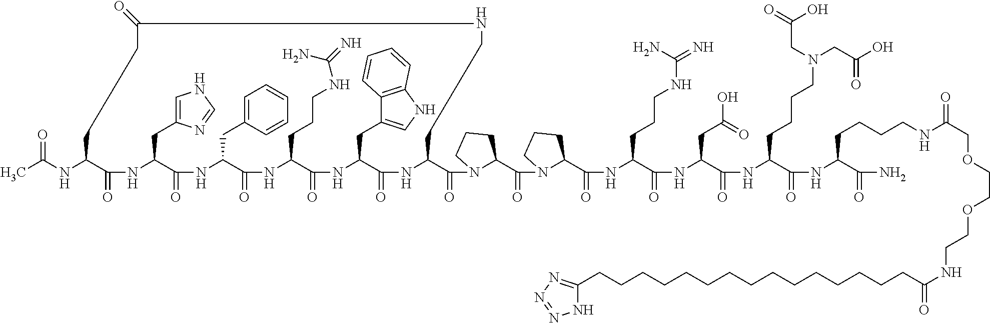 [SMILES: CC(=O)N[C@H]1CCC(=O)NCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.6 7.6 7.5 7.4 7.3 7.1 6.7 | 447 409 431 384 357 262 191 96 42 | 10 mM phos- phate |
| 17 | Ac-c[Orn- His-D- Phe-Arg- Trp-Asp]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 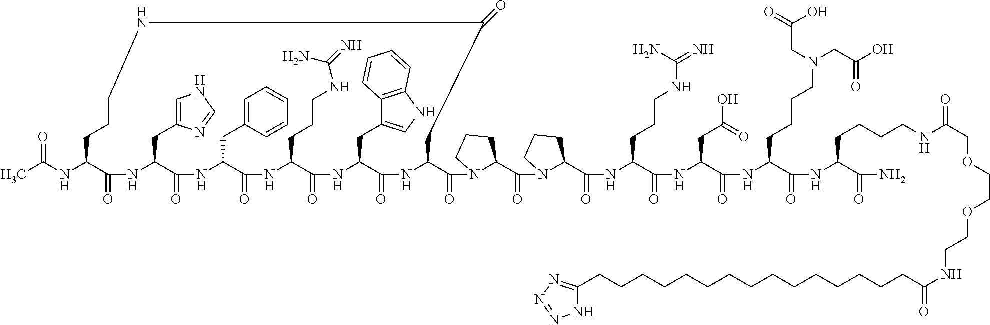 [SMILES: CC(=O)N[C@H]1CCCNC(=O)C[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.4 7.4 7.3 7.2 6.9 6.7 5.8 | 487 470 490 436 428 297 75 45 25 | 10 mM phos- phate |
| 18 | Ac-c[Glu- His-D- Phe-Arg- Trp-Orn]- Pro-Pro- Arg-Asp- Lys(bis- carboxy- methyl)- Lys({2-[2- (15- carboxy- penta- decanoyl- amino) ethoxy] ethoxy} acetyl)- NH2 | 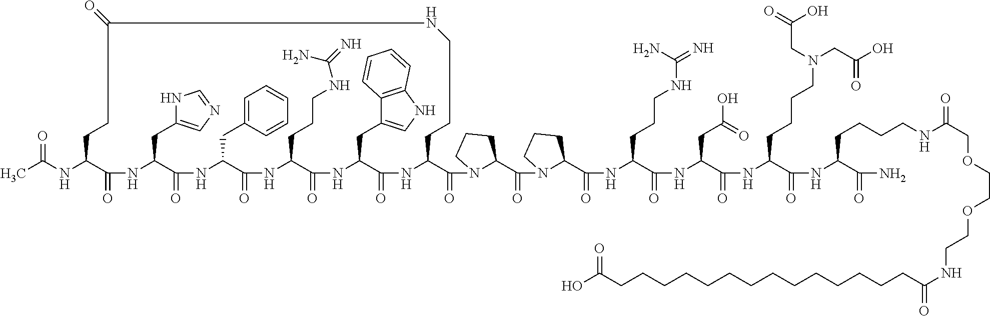 [SMILES: CC(=O)N[C@H]1CCC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O] | 7.9 7.7 7.4 7.1 6.8 6.5 6.4 6.1 5.3 | 474 451 449 484 447 392 326 310 193 | 10 mM phos- phate |
| 19 | Ac-c[Orn- His-D- Phe-Arg- Trp-Asp]- Pro-Pro- Arg-Glu- Lys(bis- carboxy- methyl)- Lys[(2-{2- [16- (tetrazol-5- yl)hexa- decanoyl- amino] ethoxy} ethoxy) acetyl]- NH2 | 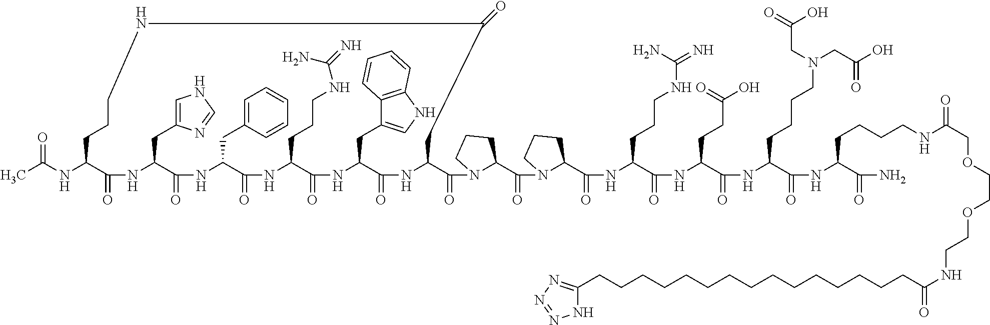 [SMILES: CC(=O)N[C@H]1CCCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.5 7.3 7.2 7.2 6.9 6.7 6.3 | 441 517 474 447 433 273 70 38 26 | 10 mM phos- phate |
| 20 | Ac-c[Orn- His-D- Phe-Arg- Trp-Glu]- Pro-Ser- Lys-Glu- Glu-Ser- Ser-Glu- Lys({2-[2- (16- carboxy- hexa- decanoyl- amino) ethoxy] ethoxy} acetyl)- NH2 | 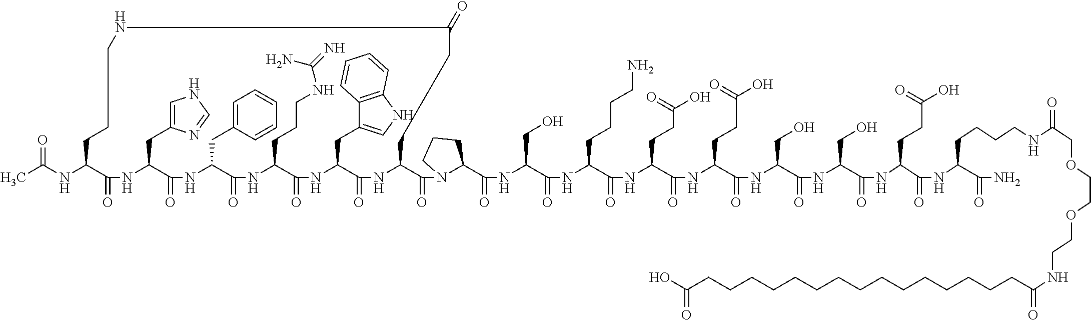 [SMILES: CC(=O)N[C@H]1CCCNC(=O)CC[C@@H](C(=O)N2CCC[C@H]2C(=O)N[C@@H](CO)C(=O)N[C@@H](CCCCN)C(=O)N[C@@H](CCC(=O)O)C(=O)N[C@@H](CCC(=O)O)C(=O)N[C@@H](CO)C(=O)N[C@@H](CO)C(=O)N[C@@H](CCC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.2 7.9 7.6 7.3 7.0 6.7 6.4 6.3 6.0 | 439 398 429 433 420 416 410 399 192 | 10 mM phos- phate |
| 21 | Ac-c[Lys- His-D- Phe-Arg- Trp-Glu]- Pro-Pro- Arg-Asp- Lys(bis- carboxy- methyl)- Lys[(2-{2- [16- (tetrazol-1- yl)hexa- decanoyl- amino] ethoxy} ethoxy) acetyl]- NH2 | 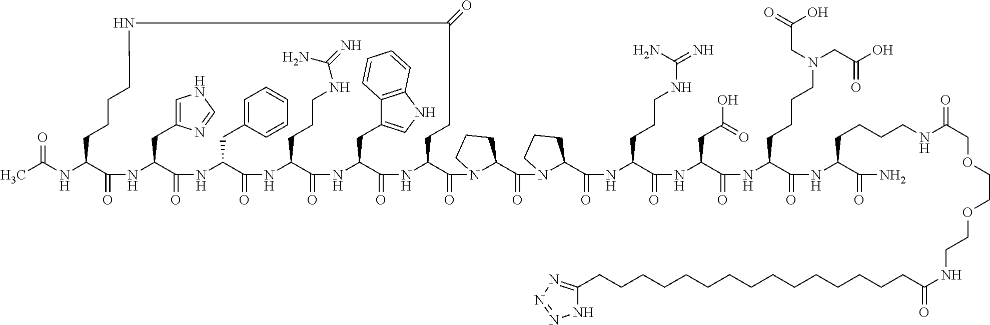 [SMILES: CC(=O)N[C@H]1CCCCNC(=O)CC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.5 7.4 7.2 6.6 6.3 6.0 5.3 | 366 360 276 175 90 47 36 31 38 | 10 mM phos- phate |
| 22 | Ac-c[Orn- His-D- Phe-Arg- Trp-Glu]- Pro-Pro- His-Glu- Lys(bis- carboxy- methyl)- Lys({2-[2- (16- carboxy- hexa- decanoyl- amino) ethoxy] ethoxy} acetyl)- NH2 | 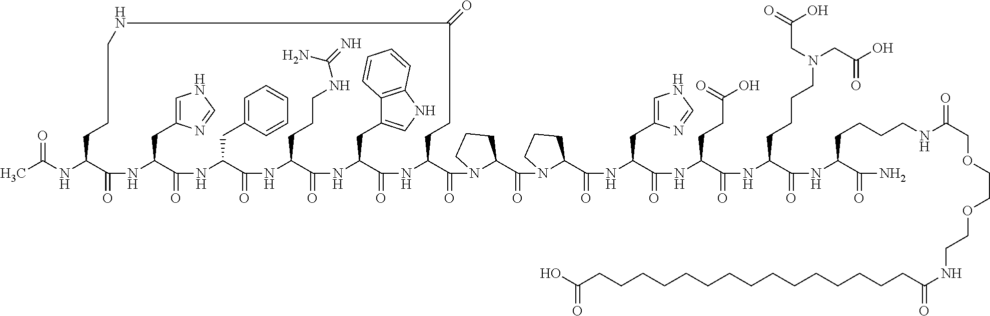 [SMILES: CC(=O)N[C@H]1CCCNC(=O)CC[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.4 7.2 6.9 6.8 6.7 6.3 6.1 | 468 438 447 443 444 284 173 134 | 10 mM phos- phate |
| 23 | Ac-c[Dab- His-D- Phe-Arg- Trp-Glu]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 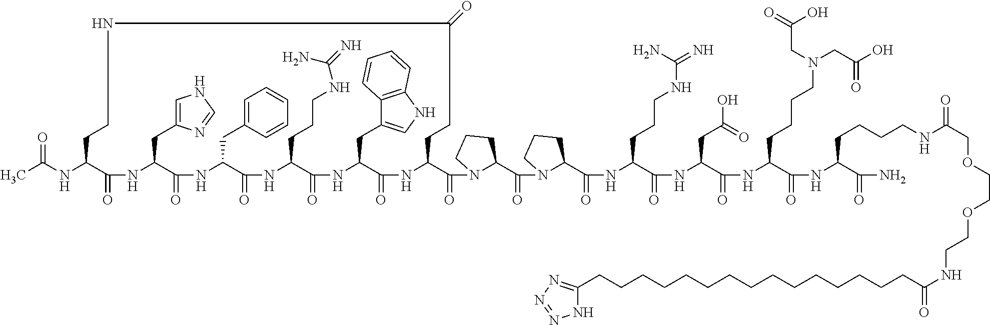 [SMILES: CC(=O)N[C@H]1CCNC(=O)CC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.6 7.4 7.2 6.9 6.6 6.1 4.7 | 300 295 229 145 76 45 38 34 73 | 10 mM phos- phate |
| 25 | Ac-c[Dab- His-D- Phe-Arg- Trp-Asp]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 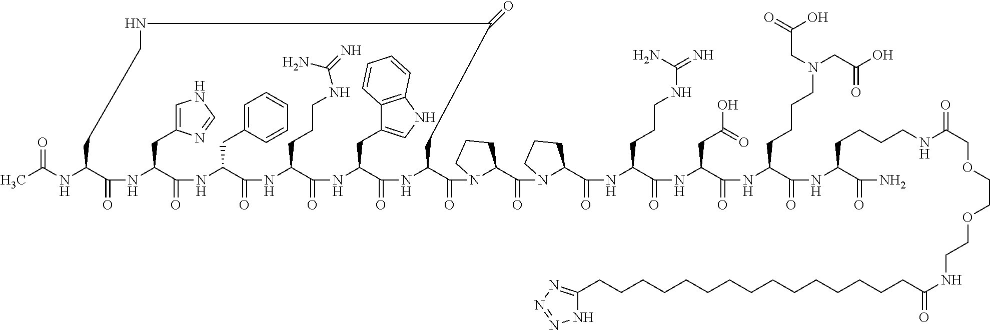 [SMILES: CC(=O)N[C@H]1CCNC(=O)C[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.7 7.5 7.4 7.3 7.3 7.0 6.3 6.0 | 498 471 479 472 439 318 133 34 24 | 10 mM phos- phate |
| 26 | Ac-c[Glu- His-D- Phe-Arg- Trp-Orn]- Pro-Pro- Asp- Lys(bis- carboxy- methyl)- Lys({2-[2- (17- carboxy- hepta- decanoyl- amino) ethoxy] ethoxy} acetyl)- NH2 | 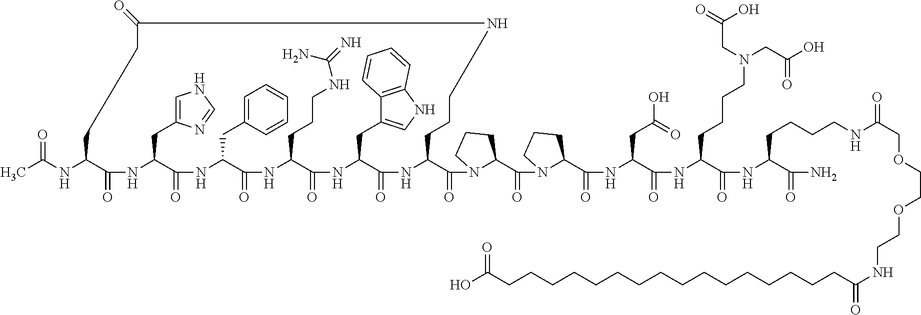 [SMILES: CC(=O)N[C@H]1CCC(=O)NCCC[C@@H](C(=O)N2CCC[C@H]2C(=O)N2CCC[C@H]2C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.4 7.1 6.8 6.5 6.1 5.8 5.5 | 478 474 482 476 461 478 474 325 49 | 10 mM phos- phate |
| 27 | Ac-c[Glu- His-D- Phe-Arg- Trp-Lys]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 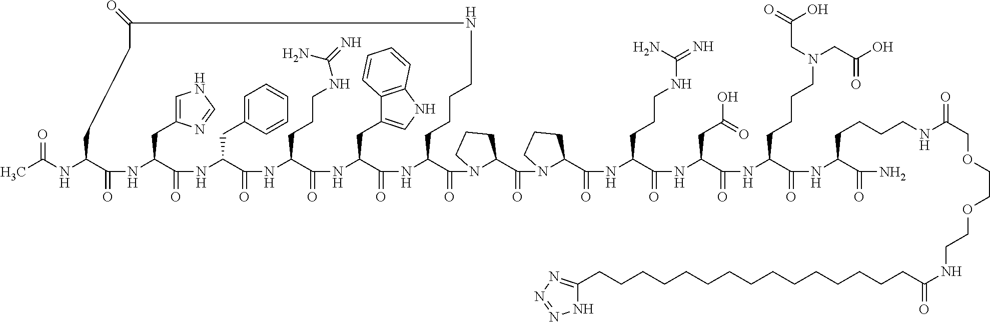 [SMILES: CC(=O)N[C@H]1CCC(=O)NCCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.8 7.6 7.5 7.4 7.3 7.2 6.9 6.6 | 435 481 521 474 459 394 263 65 42 | 10 mM phos- phate |
| 28 | Ac-c[Orn- His-D- Phe-Arg- Trp-Asp]- Pro-Ser- Ser-Ser- Lys(bis- carboxy- methyl)- Ser-Ser- Lys[(2-{2- [16- (tetrazol-5- yl)hexa- decanoyl- amino] ethoxy} ethoxy) acetyl]- NH2 | 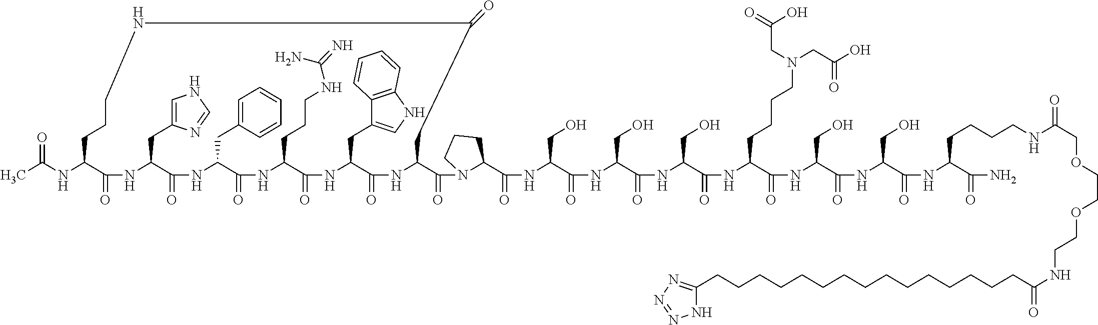 [SMILES: CC(=O)N[C@H]1CCCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.8 7.5 7.2 7.0 6.7 6.4 5.6 4.8 | 415 433 410 406 382 126 59 40 71 | 10 mM phos- phate |
| 30 | Ac-c[Asp- His-D- Phe-Arg- Trp-Dab]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 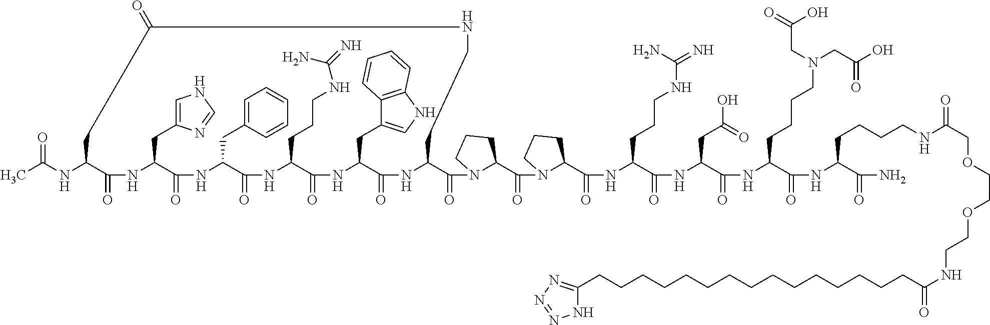 [SMILES: CC(=O)N[C@H]1CC(=O)NCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.6 7.5 7.5 7.3 7.3 7.0 6.4 5.9 | 497 493 478 476 377 318 144 37 24 | 10 mM phos- phate |
| 31 | Ac-c[Glu- His-D- Phe-Arg- Trp-Dap]- Hyp-Hyp- Arg-Asp- Lys(bis- carboxy- methyl)- Lys[(2-{2- [16- (tetrazol-5- yl)hexa- decanoyl- amino] ethoxy} ethoxy) acetyl]- NH2 | 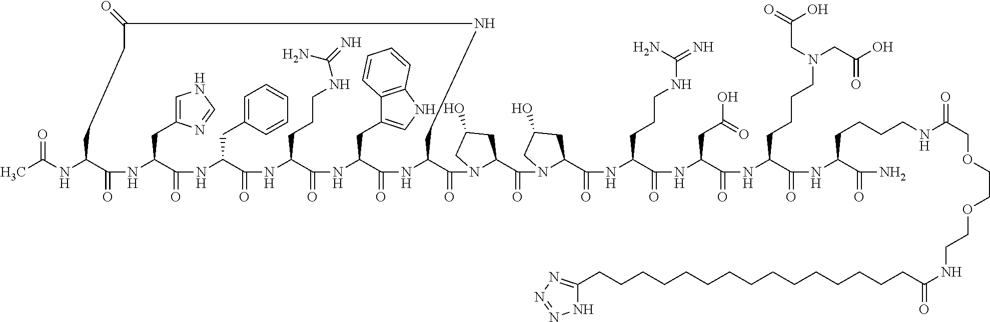 [SMILES: CC(=O)N[C@H]1CCC(=O)NC[C@@H](C(=O)N2C[C@H](O)C[C@H]2C(=O)N2C[C@H](O)C[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.7 7.5 7.5 7.4 7.3 6.9 6.6 | 472 529 495 440 368 228 96 61 36 | 10 mM phos- phate |
| 32 | Ac-c[Glu- His-D- Phe-Arg- Trp-Dab]- Hyp-Hyp- Arg-Asp- Lys(bis- carboxy- methyl)- Lys[(2-{2- [16- (tetrazol-5- yl)hexa- decanoyl- amino] ethoxy} ethoxy) acetyl]- NH2 | 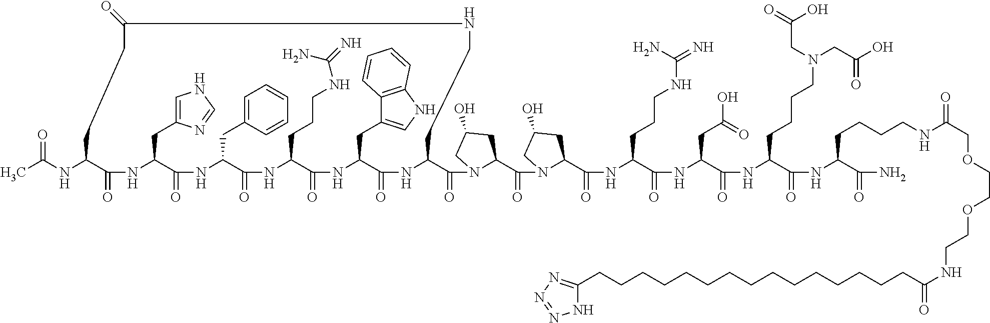 [SMILES: CC(=O)N[C@H]1CCC(=O)NCC[C@@H](C(=O)N2C[C@H](O)C[C@H]2C(=O)N2C[C@H](O)C[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.8 7.8 7.6 7.5 7.2 7.0 6.7 | 230 674 640 358 324 258 89 60 40 | 10 mM phos- phate |
| 33 | Ac-c[Dab- His-D- Phe-Arg- Trp-Asp]- Pro-Pro- His-Glu- Lys(bis- carboxy- methyl)- Lys[(2-{2- [16- (tetrazol-5- yl)hexa- decanoyl- amino] ethoxy} ethoxy) acetyl]- NH2 | 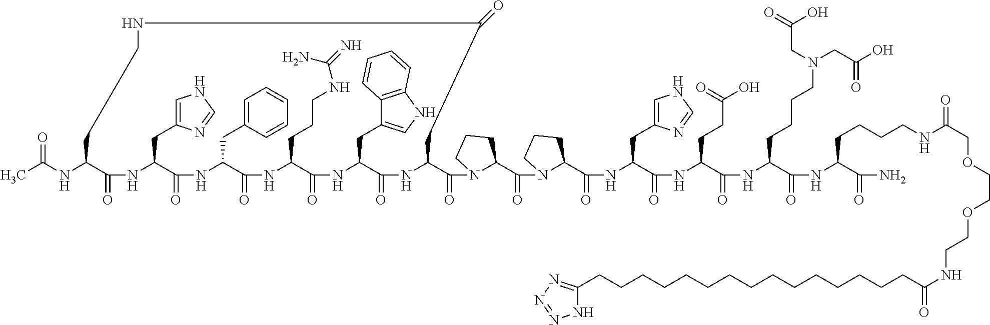 [SMILES: CC(=O)N[C@H]1CCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.6 8.1 7.8 7.5 7.3 6.9 6.7 6.6 6.4 | 473 511 489 503 488 487 485 468 122 | 10 mM phos- phate |
| 34 | Ac-c[Orn- His-D- Phe-Arg- Trp-Asp]- Pro-Pro- Arg-Glu- Lys(bis- carboxy- methyl)- Lys({2-[2- (17- carboxy- hepta- decanoyl- amino) ethoxy] ethoxy} acetyl)- NH2 | 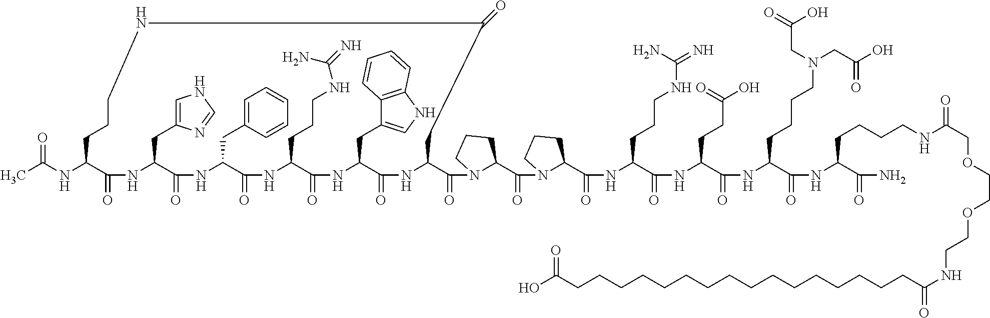 [SMILES: CC(=O)N[C@H]1CCCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.4 7.3 7.2 6.9 6.6 6.3 5.5 | 360 420 355 261 165 87 62 42 27 | 10 mM phos- phate |
| 35 | Ac-c[Asp- His-D- Phe-Arg- Trp-Lys]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 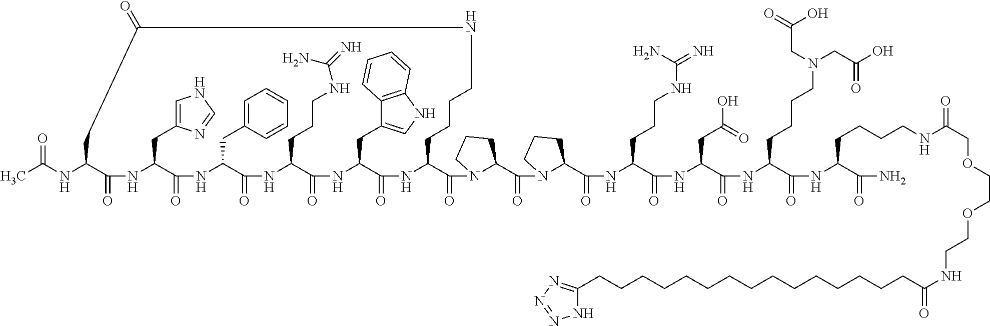 [SMILES: CC(=O)N[C@H]1CC(=O)NCCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.7 7.5 7.5 7.4 7.3 7.2 6.8 6.4 | 572 537 526 541 425 211 126 44 24 | 10 mM phos- phate |
| 36 | Ac-c[Dap- His-D- Phe-Arg- Trp-Asp]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 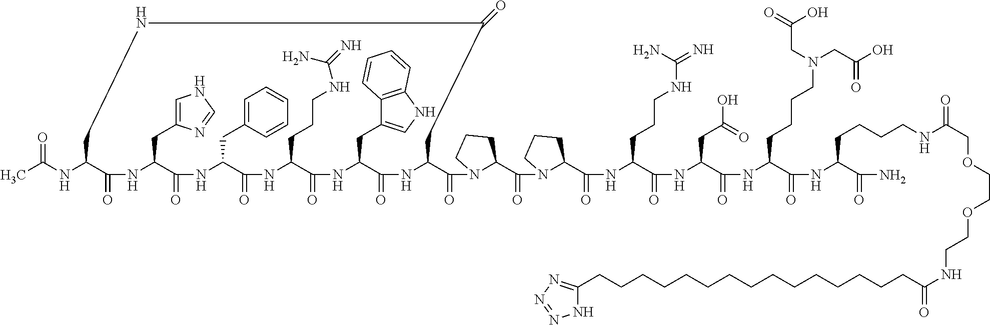 [SMILES: CC(=O)N[C@H]1CNC(=O)C[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.6 7.5 7.4 7.1 6.5 6.0 5.7 | 505 514 504 488 261 87 28 18 17 | 10 mM phos- phate |
| 37 | Ac-c[Glu- Gln-D- Phe-Arg- Trp-Orn]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 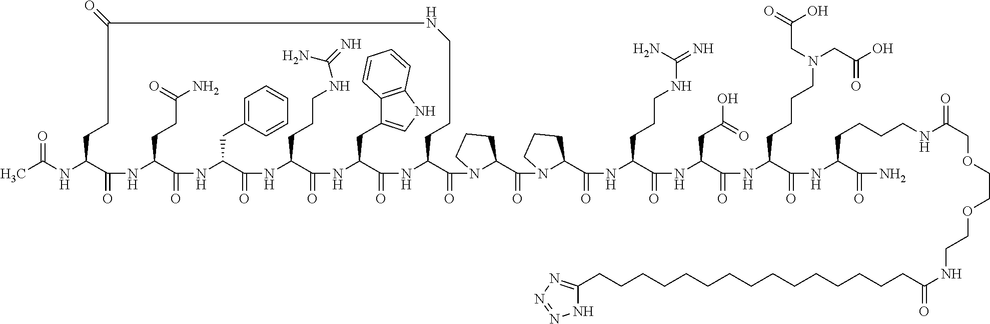 [SMILES: CC(=O)N[C@H]1CCC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](CCC(N)=O)NC1=O] | 7.9 7.6 7.5 7.3 7.2 7.0 6.8 6.6 6.4 5.7 | 507 467 458 486 482 510 453 450 464 95 | 10 mM phos- phate |
| 38 | Ac-c[Glu- His-D- Phe-Arg- Trp-Orn]- Pro-Pro- Arg-Asp- Lys(bis- carboxy- methyl)- Lys({2-[2- (17- carboxy- hepta- decanoyl- amino) ethoxy] ethoxy} acetyl)- NH2 | 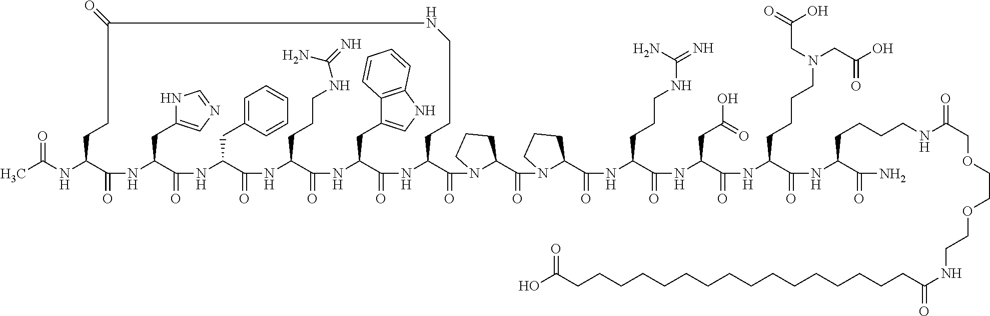 [SMILES: CC(=O)N[C@H]1CCC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O] | 7.9 7.7 7.6 7.5 7.4 7.0 6.5 6.2 5.4 | 430 422 360 282 218 111 58 36 21 | 10 mM phos- phate |
| 39 | Ac-c[Orn- His-D- Phe-Arg- Trp-Glu]- Pro-Ser- Ser-Ser- Glu-Glu- Ser-Ser- Lys({2-[2- (16- carboxy- hexa- decanoyl- amino) ethoxy] ethoxy} acetyl)- NH2 | 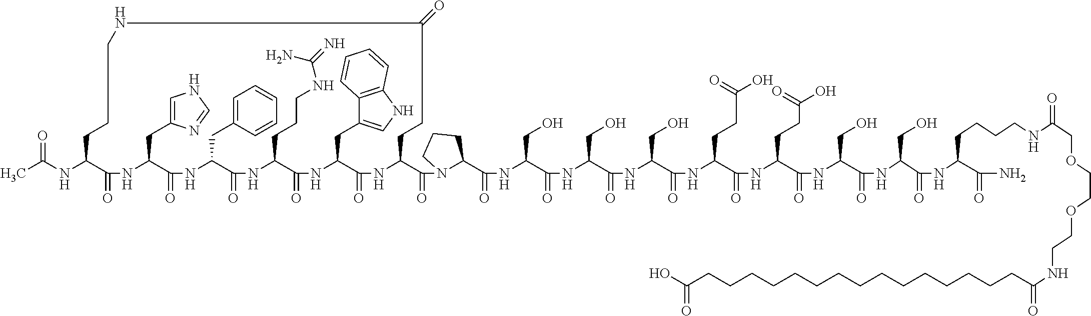 [SMILES: CC(=O)N[C@H]1CCCNC(=O)CC[C@@H](C(=O)N2CCC[C@H]2C(=O)N[C@@H](CO)C(=O)N[C@@H](CO)C(=O)N[C@@H](CO)C(=O)N[C@@H](CCC(=O)O)C(=O)N[C@@H](CCC(=O)O)C(=O)N[C@@H](CO)C(=O)N[C@@H](CO)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.3 8.0 7.7 7.4 7.1 6.9 6.6 6.1 | 445 397 426 448 419 397 403 272 | 10 mM phos- phate |
| 40 | Ac-c[Orn- Glu-His- D-Phe- Arg-Trp- Gly-Asp]- Pro-Ser- Lys-Glu- Glu-Ser- Ser- Lys({2-[2- (16- carboxy- hexa- decanoyl- amino) ethoxy] ethoxy) acetyl)- NH2 | 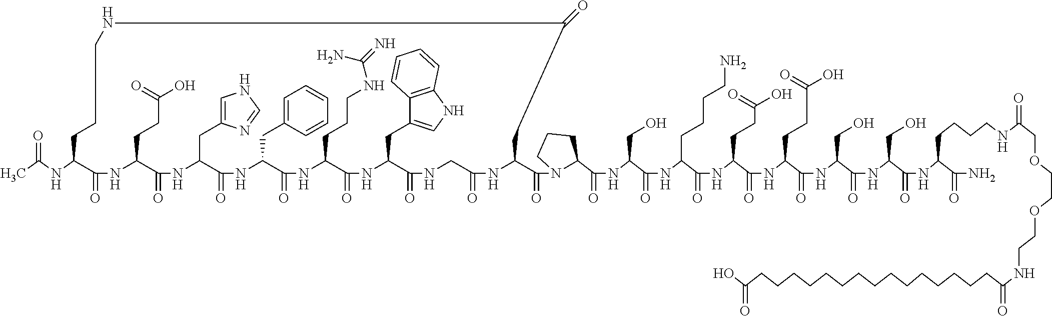 [SMILES: CC(=O)N[C@H]1CCCNC(=O)C[C@@H](C(=O)N2CCC[C@H]2C(=O)N[C@@H](CO)C(=O)NC(CCCCN)C(=O)N[C@@H](CCC(=O)O)C(=O)N[C@@H](CCC(=O)O)C(=O)N[C@@H](CO)C(=O)N[C@@H](CO)C(=O)N[C@@H](CCCCNC(=O)COCCOCCNC(=O)CCCCCCCCCCCCCCCC(=O)O)C(N)=O)NC(=O)CNC(=O)[C@H](Cc2c[nH]c3ccccc23)NC(=O)[C@H](CCCNC(=N)N)NC(=O)[C@@H](Cc2ccccc2)NC(=O)C(Cc2c[nH]cn2)NC(=O)[C@H](CCC(=O)O)NC1=O] | 8.2 8.0 7.6 7.4 7.0 6.7 6.5 6.3 5.9 | 503 488 489 499 505 493 512 501 266 | 10 mM phos- phate |
| 41 | Ac-c[Asp- His-D- Phe-Arg- Trp-Dap]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 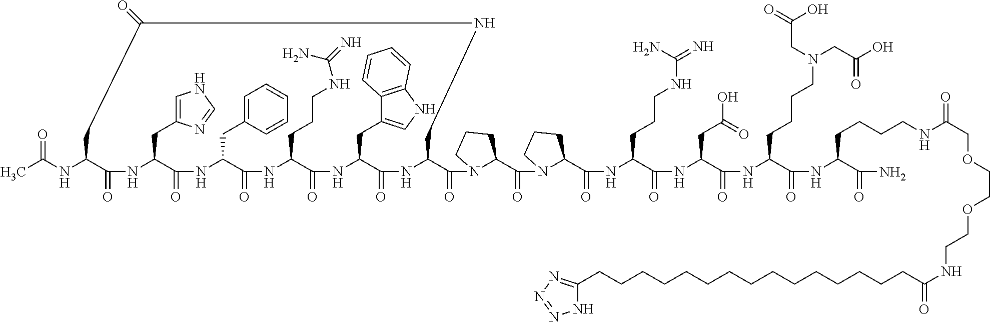 [SMILES: CC(=O)N[C@H]1CC(=O)NC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.7 7.6 7.5 7.5 7.4 7.3 7.0 | 493 451 478 497 387 336 160 87 39 | 10 mM phos- phate |
| 42 | Ac-c(Orn- His-D- Phe-Arg- Trp-Asp]- Pro-Pro- Arg-Glu- Lys(bis- carboxy- methyl)- Lys({2-[2- (15- carboxy- penta- decanoyl- amino) ethoxy] ethoxy} acetyl)- NH2 | 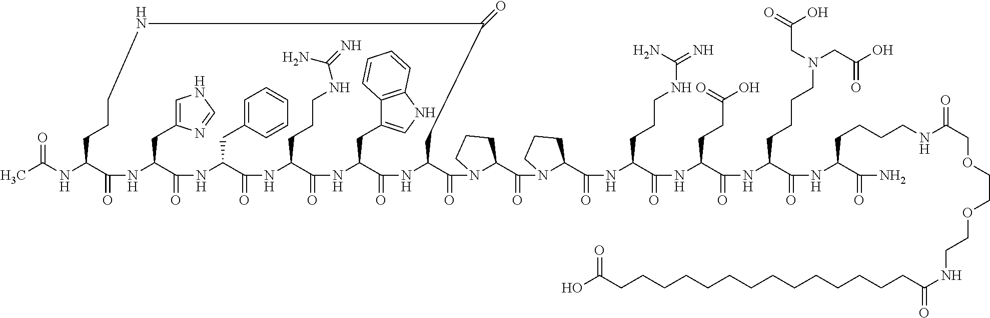 [SMILES: CC(=O)N[C@H]1CCCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.5 7.2 7.0 6.7 6.4 5.8 4.9 | 417 476 441 438 449 421 364 240 153 | 10 mM phos- phate |
| 43 | Ac-c[Glu- His-D- Phe-Arg- Trp-Orn]- Pro-Pro- His-Asp- Lys(bis- carboxy- methyl)- Lys[(2-{2- [16- (tetrazol-5- yl)hexa- decanoyl- amino] ethoxy} ethoxy) acetyl]- NH2 | 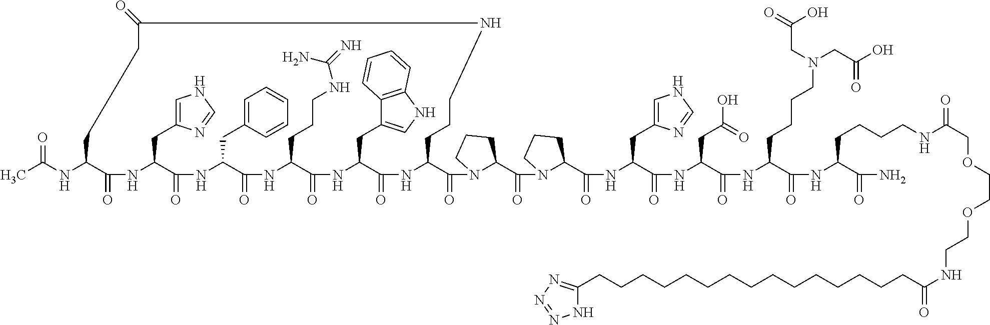 [SMILES: CC(=O)N[C@H]1CCC(=O)NCCC[C@@H](C(=O)N2CCC[C@H]2C(=O)N2CCC[C@H]2C(=O)N[C@@H](Cc2c[nH]cn2)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.7 7.4 7.2 6.8 6.7 6.5 5.9 | 493 490 487 506 478 478 315 75 27 | 10 mM phos- phate |
| 44 | Ac-c[Lys- His-D- Phe-Arg- Trp-Asp]- Pro-Pro- His-Glu- Lys(bis- carboxy- methyl)- Lys({2-[2- (16- carboxy- hexa- decanoyl- amino) ethoxy] ethoxy} acetyl)- NH2 | 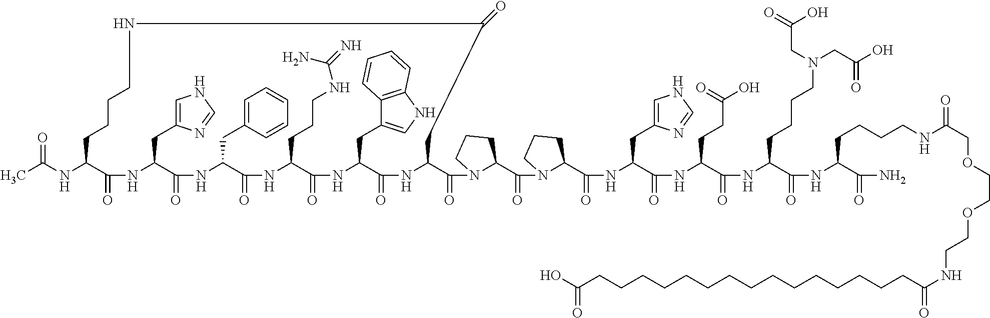 [SMILES: CC(=O)N[C@H]1CCCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.2 8.0 7.8 7.6 7.3 7.0 6.8 6.7 6.5 | 430 408 429 407 412 430 407 364 227 | 10 mM phos- phate |
| 45 | Ac-c[Lys- His-D- Phe-Arg- Trp-Asp]- Pro-Pro- His-Ser- Lys(bis- carboxy- methyl)- Ser-Ser- Glu- Lys({2-[2- (16- carboxy- hexa- decanoyl- amino) ethoxy] ethoxy} acetyl)- NH2 | 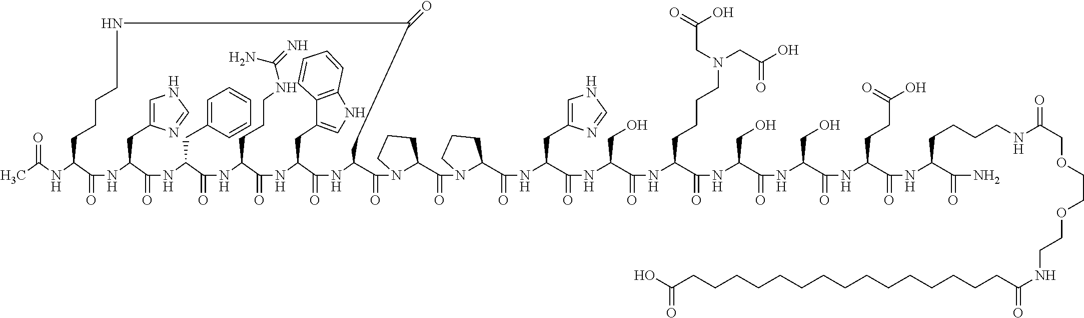 [SMILES: CC(=O)N[C@H]1CCCCNC(=O)C[C@@H](C(=O)N2CCC[C@H]2C(=O)N2CCC[C@H]2C(=O)N[C@@H](Cc2c[nH]cn2)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.1 7.9 7.7 7.5 7.3 7.0 6.8 6.7 6.4 | 400 391 379 380 391 370 350 334 199 | 10 mM phos- phate |
| 46 | Ac-c[Orn- His-D- Phe-Arg- Trp-Asp]- Pro-Pro- His-Glu- Lys(bis- carboxy- methyl)- Lys({2-[2- (17- carboxy- hepta- decanoyl- amino) ethoxy] ethoxy} acetyl)- NH2 | 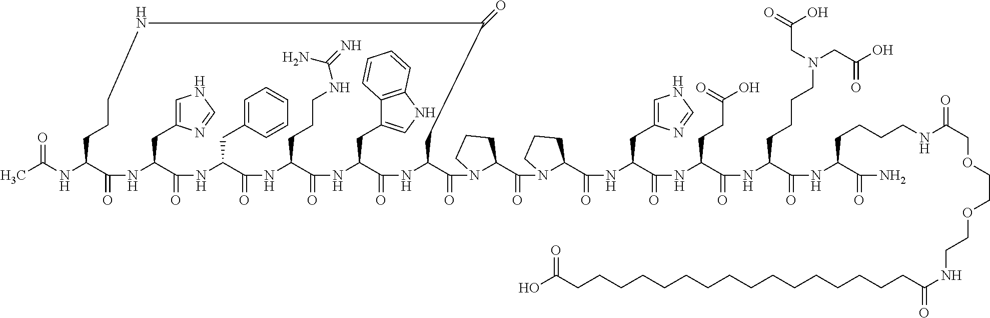 [SMILES: CC(=O)N[C@H]1CCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.8 7.6 7.1 6.8 6.7 6.6 6.1 5.4 5.0 | 507 509 494 471 422 160 47 24 25 | 10 mM phos- phate |
| 47 | Ac-c[Dap- His-D- Phe-Arg- Trp-Glu]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 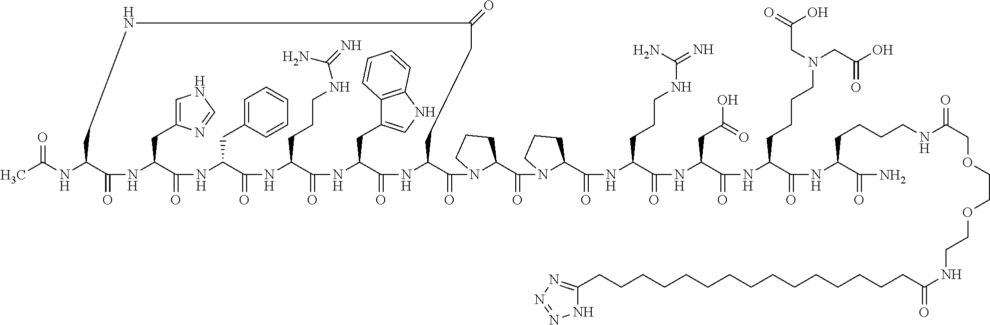 [SMILES: CC(=O)N[C@H]1CNC(=O)CC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.6 7.5 7.3 7.2 7.1 6.9 6.4 | 493 371 379 366 219 103 56 24 | 10 mM phos- phate |
| 48 | Ac-c[Glu- His-D- Phe-Arg- Trp-Lys]- Hyp-Hyp- Arg-Asp- Lys(bis- carboxy- methyl)- Lys[(2-{2- [16- (tetrazol-5- yl)hexa- decanoyl- amino] ethoxy} ethoxy) acetyl]- NH2 | 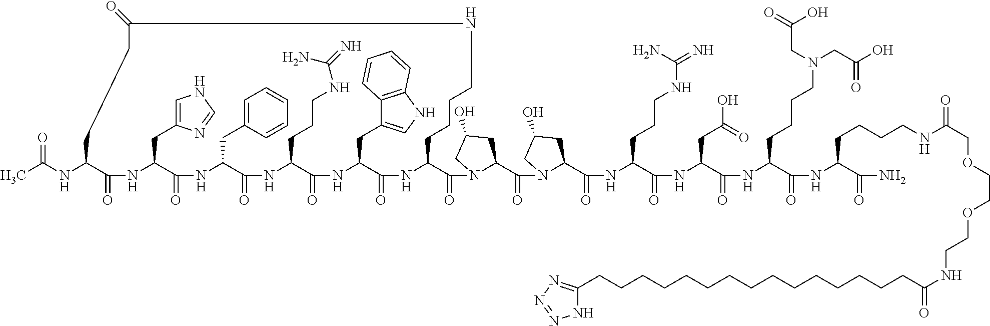 [SMILES: CC(=O)N[C@H]1CCC(=O)NCCCC[C@@H](C(=O)N2C[C@H](O)C[C@H]2C(=O)N2C[C@H](O)C[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.3 8.1 8.0 7.8 7.6 7.5 7.4 7.3 7.0 6.7 | 412 418 420 430 416 368 309 218 107 54 | 10 mM phos- phate |
| 49 | Ac-c[Lys- His-D- Phe-Arg- Trp-Asp]- Pro-Ser- His-Glu- Lys(bis- carboxy- methyl)- Lys({2-[2- (16- carboxy- hexa- decanoyl- amino) ethoxy] ethoxy} acetyl)- NH2 | 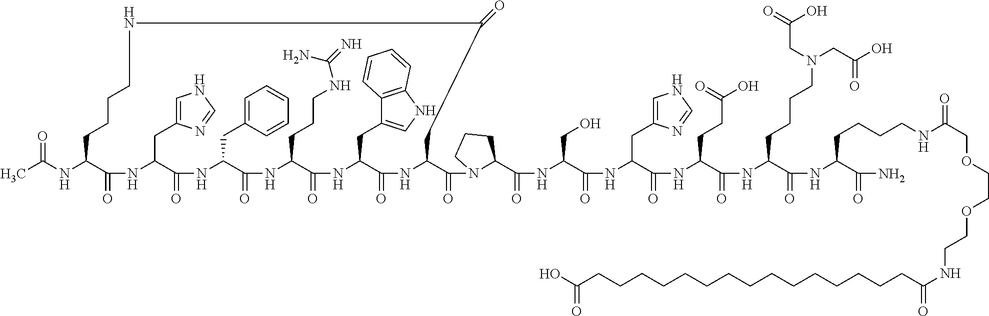 [SMILES: CC(=O)N[C@H]1CCCCNC(=O)C[C@@H](C(=O)N2CCC[C@H]2C(=O)N[C@@H](CO)C(=O)NC(Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)C(Cc2c[nH]cn2)NC1=O] | 8.2 8.0 7.8 7.7 7.3 7.0 6.8 6.6 6.5 | 446 410 456 439 460 462 427 423 248 | 10 mM phos- phate |
| 50 | Ac-c[Orn- His-D- Phe-Arg- Trp-Asp]- Pro-Pro- Arg-Glu- Lys(bis- carboxy- methyl)- Ser-Ser- NH(2-{2- [2-(17- carboxy- hepta- decaonyl- amino) ethoxy] ethoxy} ethyl) | 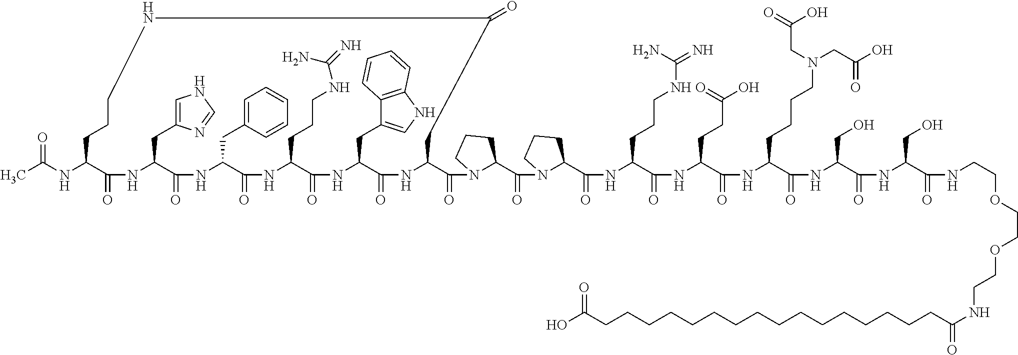 [SMILES: CC(=O)N[C@H]1CCCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.2 7.9 7.8 7.7 7.5 7.4 7.2 7.0 6.5 | 212 208 212 197 176 156 123 92 57 | 10 mM phos- phate |
| 51 | Ac-c[Orn- His-D- Phe-Arg- Trp-Asp]- Pro-Pro- His-Glu- Lys(bis- carboxy- methyl)- Lys({2-[2- (15- carboxy- penta- decanoyl- amino) ethoxy] ethoxy} acetyl)- NH2 | 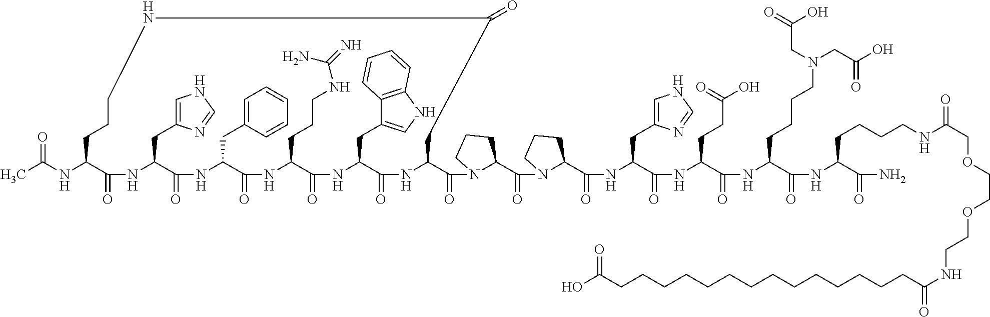 [SMILES: CC(=O)N[C@H]1CCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.8 7.6 7.3 7.0 6.7 6.4 6.1 5.8 5.3 | 457 485 468 442 460 450 359 280 204 | 10 mM phos- phate |
| 52 | Ac-c[Lys- His-D- Phe-Arg- Trp-Asp]- Pro-Pro- His-Glu- Glu-Ser- Ser-Glu- Lys({2-[2- (16- carboxy- hexa- decanoyl- amino) ethoxy] ethoxy} acetyl)- NH2 | 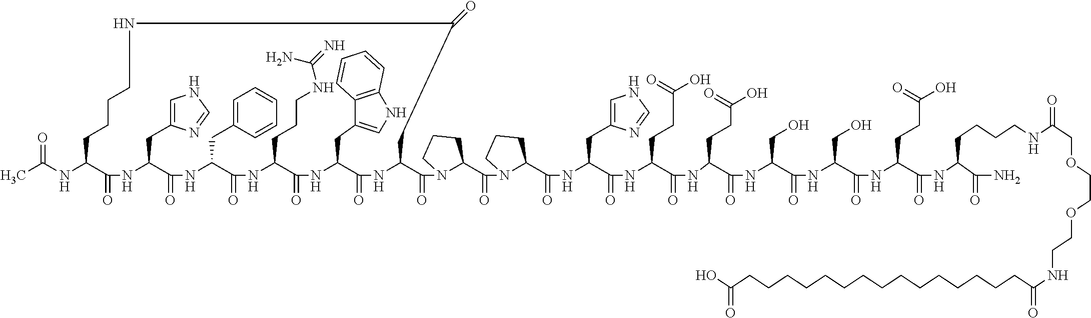 [SMILES: CC(=O)N[C@H]1CCCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCC(=O)O)C(=O)N[C@@H](CO)C(=O)N[C@@H](CO)C(=O)N[C@@H](CCC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.3 8.0 7.6 7.5 7.3 7.0 6.7 6.3 6.1 | 390 391 381 382 378 372 376 339 169 | 10 mM phos- phate |
| 53 | Ac-c[Lys- His-D- Phe-Arg- Trp-Asp]- Pro-Ser- Ser-Ser- Lys(bis- carboxy- methyl)- Ser-Ser- NH(2-{2- [2-(17- carboxy- hepta- decanoyl- amino) ethoxy] ethoxy} ethyl | 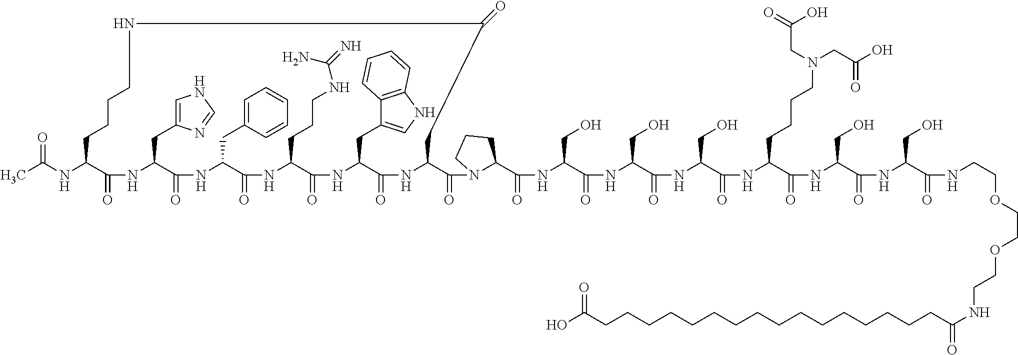 [SMILES: CC(=O)N[C@H]1CCCCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.2 7.9 7.7 7.5 7.4 7.3 7.2 6.9 6.6 | 403 425 393 385 410 396 337 159 82 | 10 mM phos- phate |
| 54 | Ac-c[Glu- His-D- Phe-Arg- Trp-Orn]- Pro-Pro- Arg-Asp- Lys(bis- carboxy- methyl)- Lys[(2-{2- [16- (tetrazol-5- yl)hexa- decanoyl- amino] ethoxy} ethoxy) acetyl]- NH2 | 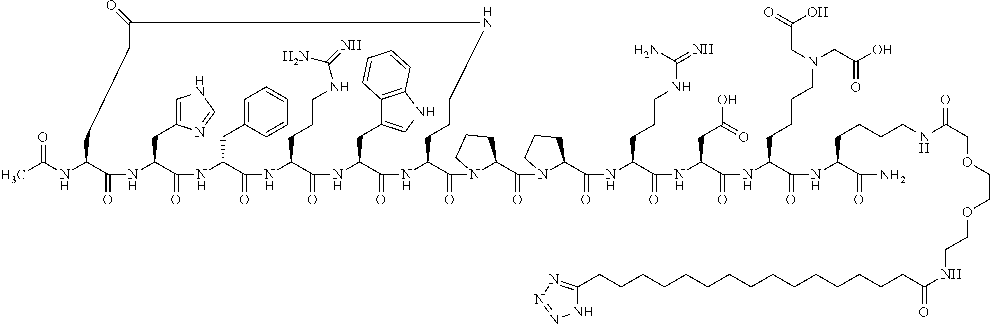 [SMILES: CC(=O)N[C@H]1CCC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.8 7.6 7.4 7.1 7.0 6.8 6.2 5.5 | 217 200 202 221 95 78 43 23 22 | 10 mM phos- phate |
| 55 | Ac-c[Orn- His-D- Phe-Arg- Trp-Asp]- Pro-Pro- His-Glu- Lys(bis- carboxy- methyl)- Lys({2-[2- (16- carboxy- hexa- decanoyl- amino) ethoxy] ethoxy} acetyl)- NH2 | 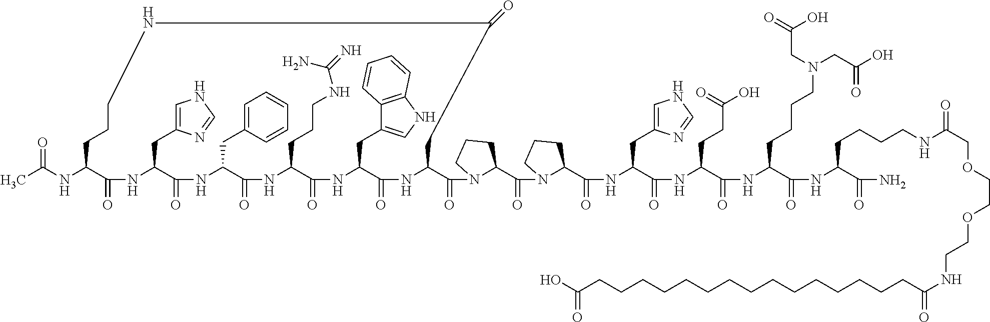 [SMILES: CC(=O)N[C@H]1CCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.7 7.6 7.2 6.9 6.7 6.6 6.5 6.3 | 426 435 422 414 426 436 337 236 152 | 10 mM phos- phate |
| 56 | Ac-c[Glu- His-D- Phe-Arg- Trp-Orn]- Hyp-Hyp- Arg-Asp- Lys(bis- carboxy- methyl)- Lys[(2-{2- [16- (tetrazol-5- yl)hexa- decanoyl- amino] ethoxy} ethoxy) acetyl]- NH2 | 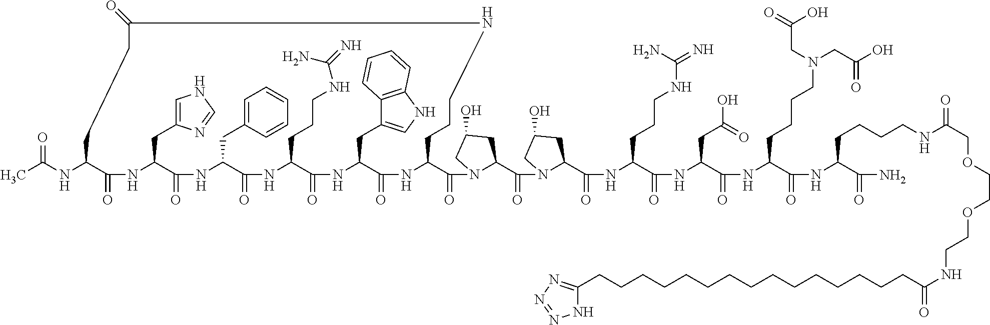 [SMILES: CC(=O)N[C@H]1CCC(=O)NCCC[C@@H](C(=O)N2C[C@H](O)C[C@H]2C(=O)N2C[C@H](O)C[C@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.7 7.6 7.5 7.5 7.4 7.1 6.8 | 392 524 408 521 395 284 169 79 47 | 10 mM phos- phate |
| 57 | Ac-c[Orn- His-D- Phe-Arg- Trp-Glu]- Pro-Ser- Ser-Ser- Lys(bis- carboxy- methyl)- Ser-Ser- Lys({2-[2- (16- carboxy- hexa- decanoyl- amino) ethoxy] ethoxy} acetyl)- NH2 | 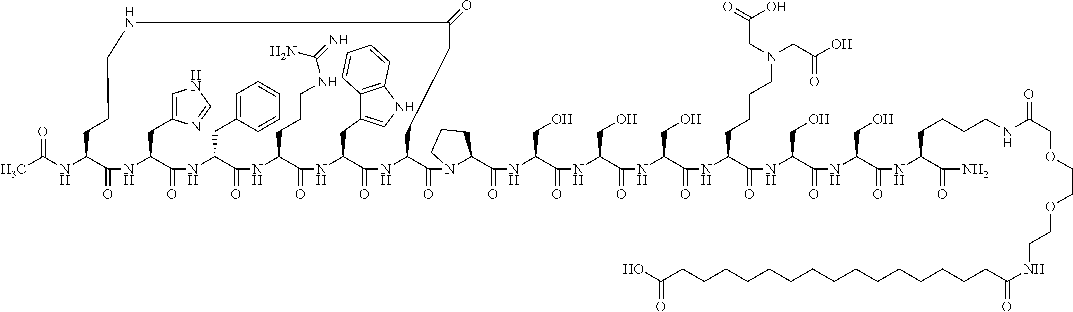 [SMILES: CC(=O)N[C@H]1CCCNC(=O)CC[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.7 7.6 7.5 7.5 7.4 7.1 6.8 | 392 524 408 521 395 284 169 79 47 | 10 mM phos- phate |
| 58 | Ac-c [homoCys- Ser-D- Phe-Arg- Trp-Cys]- Pro-Pro- Lys-Glu- Lys(bis- carboxy- methyl)- Lys[(2-{2- [16- (tetrazol-5- yl)hexa- decanoyl- amino] ethoxy} ethoxy) acetyl]- NH2 | 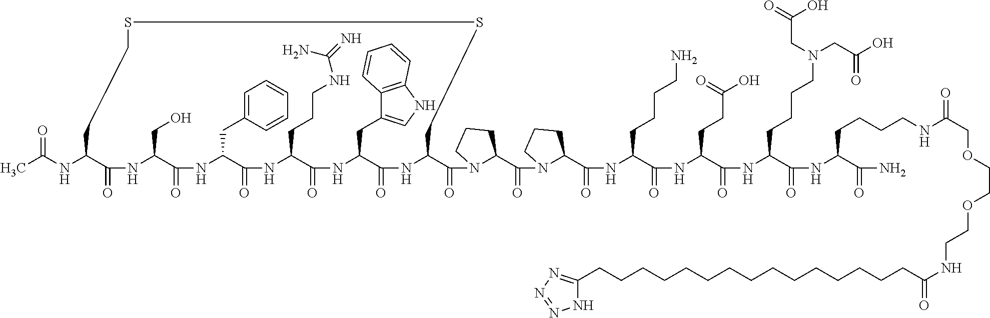 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](CO)NC1=O] | 9.0 8.6 7.6 7.5 6.6 6.5 6.2 5.9 3.9 3.0 | 365 250 385 80 30 37 21 8 56 539 | Water |
| 60 | Ac-c [homoCys- His-D- Phe-Arg- Trp-Cys]- Ser-Pro- Lys-Glu- Lys(bis- carboxy- methyl)- Lys({2-[2- (15- carboxy- penta- decanoyl- amino) ethoxy] ethoxy} acetyl)- NH2 | 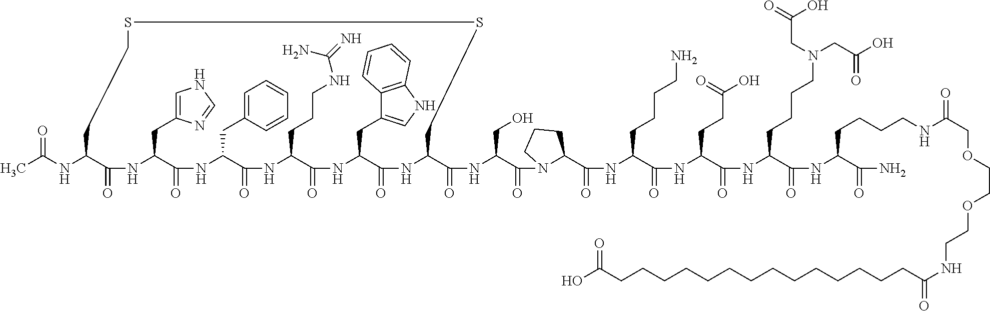 [SMILES: CC(=O)N[C@H]1CCSSC[C@@H](C(=O)N[C@@H](CO)C(=O)N2CCC[C@H]2C(=O)N[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.9 7.8 7.6 7.3 6.8 6.4 6.2 6.0 5.4 4.0 | 450 461 456 459 457 415 171 133 126 424 | Water |
| 61 | Ac-c [homoCys- His-D- Phe-Arg- Trp-Cys]- Pro-Pro- Lys-Asp- Dap{[bis (carboxy- methyl) amino] acetyl}- Lys[(2-{2- [16- (tetrazol-5- yl)hexa- decanoyl- amino] ethoxy} ethoxy) acetyl]- NH2 | 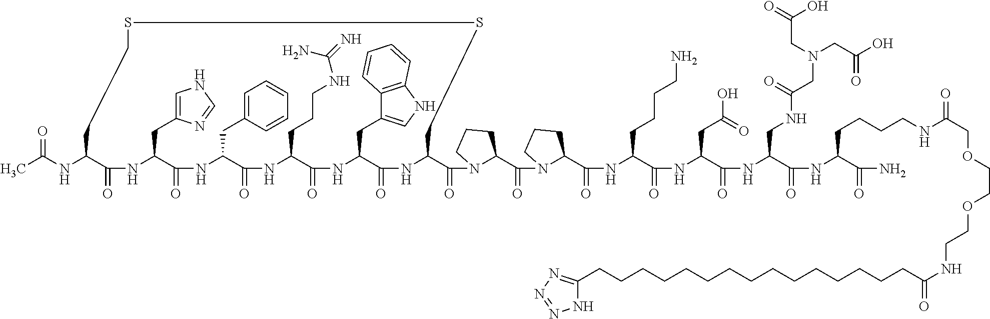 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 7.7 7.4 7.3 7.1 6.8 6.3 5.3 4.8 4.4 3.3 | 805 713 630 256 46 26 32 584 575 623 | Water |
| 62 | Ac-c [homoCys- His-D- Phe-Arg- Trp-Cys]- Ser-Pro- His-Glu- Lys(bis- carboxy- methyl)- Lys({2-[2- (17- carboxy- hepta- decanoyl- amino) ethoxy] ethoxy} acetyl)- NH2 | 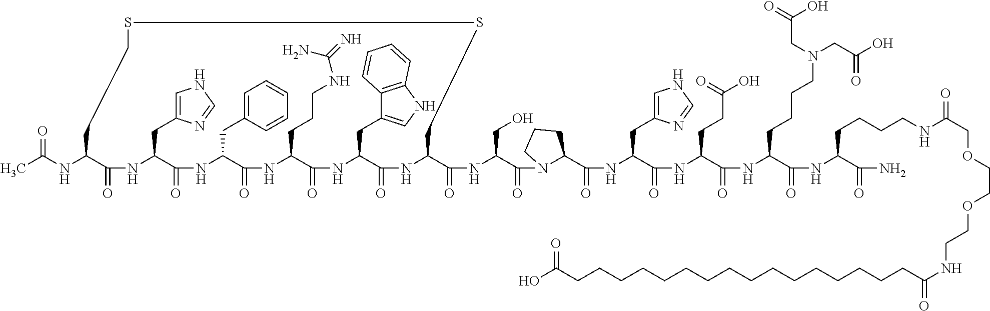 [SMILES: CC(=O)N[C@H]1CCSSC[C@@H](C(=O)N[C@@H](CO)C(=O)N2CCC[C@H]2C(=O)N[C@@H](Cc2c[nH]cn2)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.9 7.7 7.4 7.3 7.0 6.9 6.7 5.0 3.4 | 233 245 251 243 241 42 82 41 15 235 | water |
| 63 | Ac-c[Cys- Glu-His- D-Phe- Arg-Trp- Cys]-Pro- Pro-Lys- Glu- Lys(bis- carboxy- methyl)- Lys[(2-{2- [16- (tetrazol-5- yl)hexa- decanoyl- amino] ethoxy} ethoxy) acetyl]- NH2 | 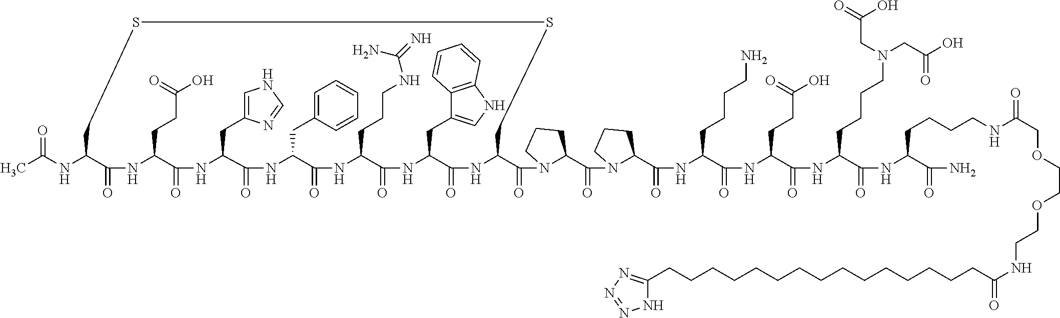 [SMILES: CC(=O)N[C@H]1CSSC[C@@H](C(=O)N2CCC[C@H]2C(=O)N2CCC[C@H]2C(=O)N[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@H](CCC(=O)O)NC1=O] | 8.9 8.5 8.1 7.9 6.0 6.0 5.1 4.6 4.2 3.8 | 465 495 417 451 484 388 78 28 23 33 | Water |
| 66 | Ac-c [homoCys- Gln-D- Phe-Arg- Trp-Cys]- Pro-Pro- Lys-Asp- Dap{[bis carboxy- methyl) amino] acetyl}- Lys[(2-{2- [16- (tetrazol-5- yl)hexa- decanoyl- amino] ethoxy} ethoxy) acetyl]- NH2 | 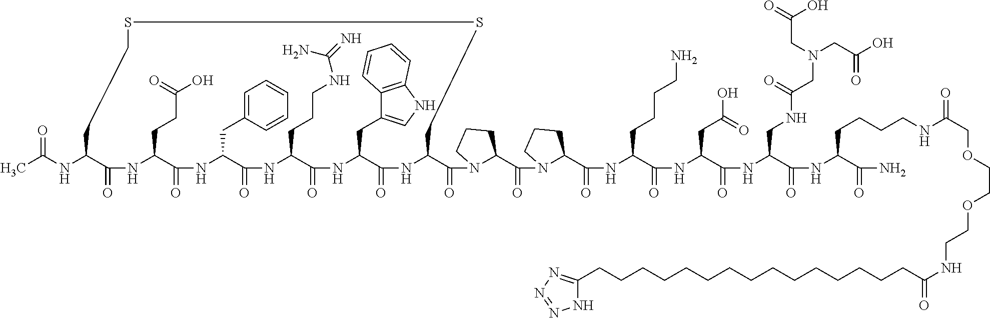 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](CCC(=O)O)NC1=O] | 8.1 7.6 7.4 7.2 6.9 6.7 6.5 6.4 6.4 4.8 | 499 507 478 461 455 447 474 390 396 38 | Water |
| 67 | Ac-c [homoCys- Hyp-D- Phe-Arg- Trp-Cys]- Pro-Pro- Lys-Asp- Dap{[bis (carboxy- methyl) amino] acetyl}- Lys[(2-{2- [16- (tetrazol-5- yl)hexa- decanoyl- amino] ethoxy} ethoxy) acetyl]- NH2 | 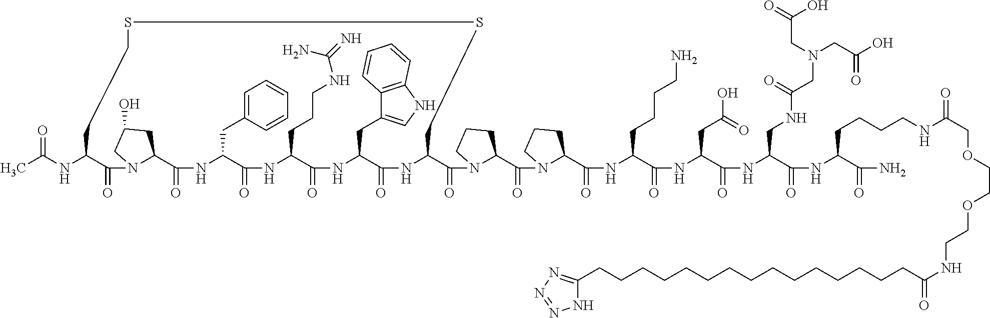 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@@H]2C[C@@H](O)CN2C1=O] | 7.3 6.8 6.4 6.3 5.9 5.4 4.1 3.6 3.4 3.3 | 590 587 533 482 207 38 37 250 420 600 | Water |
| 68 | Ac-c[Cys- Glu-His- D-Phe- Arg-Trp- Gly-Cys]- Pro-Pro- Lys-Glu- Glu- Lys({2-[2- (15- carboxy- penta- decanoyl- amino) ethoxy] ethoxy} acetyl)- NH2 | 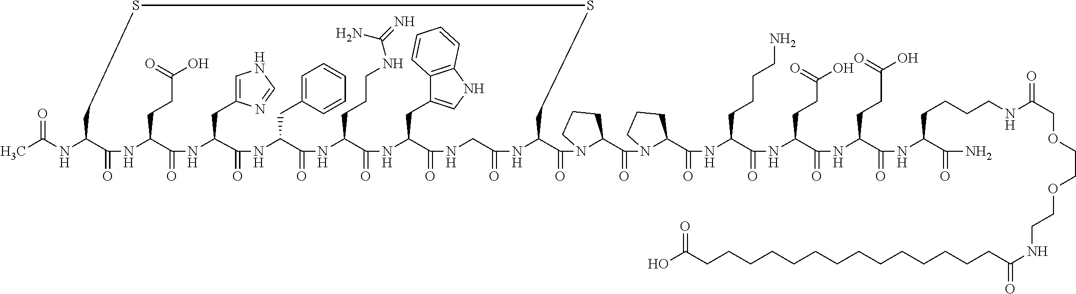 [SMILES: CC(=O)N[C@H]1CSSC[C@@H](C(=O)N2CCC[C@H]2C(=O)N2CCC[C@H]2C(=O)N[C@@H](CCCCN)C(=O)N[C@@H](CCC(=O)O)C(=O)N[C@@H](CCC(=O)O)C(=O)N[C@@H](CCCCNC(=O)COCCOCCNC(=O)CCCCCCCCCCCCCCC(=O)O)C(N)=O)NC(=O)CNC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@H](CCC(=O)O)NC1=O] | 8.0 7.9 7.5 7.3 6.9 6.9 6.2 5.8 5.1 4.0 | 183 188 184 181 182 177 164 102 87 167 | Water |
| 70 | Ac-c [homoCys- His-D- Phe-Arg- Trp-Cys]- Pro-Pro- Ser-Glu- Lys(bis] carboxy- methyl)- Lys({2-[2- (15- carboxy- penta- decanoyl- amino) ethoxy] ethoxy} acetyl)- NH2 | 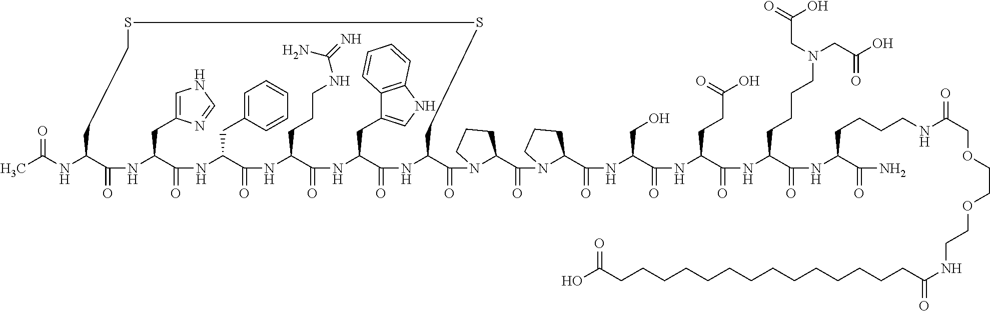 [SMILES: CC(=O)N[C@H]1CCSSC[C@@H](C(=O)N2CCC[C@H]2C(=O)N2CCC[C@H]2C(=O)N[C@@H](CO)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 8.0 7.8 7.5 6.1 5.8 5.6 5.5 4.4 3.9 3.4 | 440 463 444 429 210 142 59 40 65 190 | Water |

### Pharmacological Methods

Assay (I)—Experimental Protocol for Efficacy Testing on Appetite with MC4 Analogues, Using an Ad Libitum Fed Rat Model.

Male Sprague Dawley rats from Taconic Europe were used for the experiments. The rats had a body weight of 200-250 g at the start of experiment. The rats arrive at least 10-14 days before start of experiment with a body weight of 180-200 g. Immediately after arrival and two weeks before dosing the rats were housed in reversed light cycle (dark from 10 am to 10 pm two rats in each cage). One week before dosing, rats were moved to the FeedWin system, where the rats were placed in individual cages for acclimatisation. Since rats normally initiate food intake when light is removed, and eat the major part of their daily food intake during the night, this set up results in an alteration of the initiation time for food intake to 7:30 am, when lights were switched off. During the acclimatization period of 10-14 days, the rats had free access to food and water. During that period the animals were handled at least 3 times. Each dose of compound was tested in a group of 8 rats. A vehicle group of 8 rats was included in each set of testing.

Immediately before dosing the rats are randomised to the various treatment groups (n=5-7) by body weight. Rats are dosed in the morning before onset of dark at 10 am with a 1-3 mg/kg solution administered subcutaneously (sc). Data were recorded for 48 hours and illustrated per dose group as accumulated food intake as a function of time. At the end of the experimental session, the animals were euthanised. Results describe reduction of food intake of the test group as a percentages of the mean food intake of the vehicle group.

Rats (app. 250 g) were dosed at day 1 of the experiment, according to the table below, in the morning before onset of light. After dosing water-, food-intake and activity were registered by the FeedWin system. Data was collected each 15 minutes for 48 hours.

The effects on food intake was measured using the FeedWin system (Ellegårds Systems, Faaborg, Denmark) which contains 32 stations for individual and continuous registration of food and water intakes. One station was defined by 1 cage with a metal lid plus 2 scales, one for food-intake and one for water-intake. Food and water intake was estimated by measurements of the disappearance of preloaded amounts of food and water that were placed on the 2 scales on each side of the cage. Food intake data were recorded electronically. Movements of the animal during recordings of food intake were registered by passive infrared sensors (PIR) placed on top of the cage. These sensors detect body heat within a protected area. In addition two PIR cameras (one for food and one for water) register the time and number of episodes spent eating and drinking. The stations were linked to 2 PC's where a specially designed programme (FeedWin) collected, registered and processed all results.

SYSTEM 1, scantainer 1+2, cage 1A to 16A
SYSTEM 2, scantainer 3+4, cage 1B to 16B

The individual data were recorded in Microsoft excel sheets. Outliers were excluded after applying the Grubbs statistical evaluation test for outliers and the result were presented graphically using the GraphPad Prism program. Results describe reduction of food intake as a percentage of the mean food intake of the vehicle group. Results for 24, and 48 hours, respectively are given.

TABLE 2
In vivo efficacy testing on appetite dosing 1 mg/kg of MC4 agonists

| | | | Assay (I) | |
|---|---|---|---|---|
| | | | Food | |
| | | | con- | |
| Ex- | | | sumption | |
| am- | | | (% of | |
| ple | | | vehicle) | |
| nr. | Compound | Molecule | 24 h | 48 h |
| 3 | Ac-c[Dab-His-D- Phe-Arg-Trp-Asp]- Pro-Ser-Ser-Ser-Lys (biscarboxymethyl)- Ser-Ser-NH(2- {2-[2-(17- carboxyhepta- decanoylamino) ethoxy]ethoxy} ethyl) | 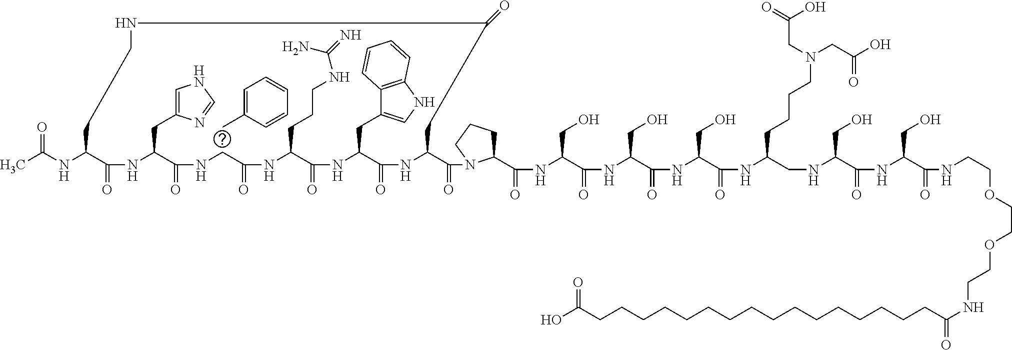 | 64 | 75 |
| 10 | Ac-c[Orn-His-D- Phe-Arg-Trp- Asp]-Pro- Pro-His-Glu-Lys (biscarboxymethyl)- Lys[(2-{2- [16-(tetrazol-5-yl) hexadecanoylamino] ethoxy}ethoxy) acetyl]-NH2 | 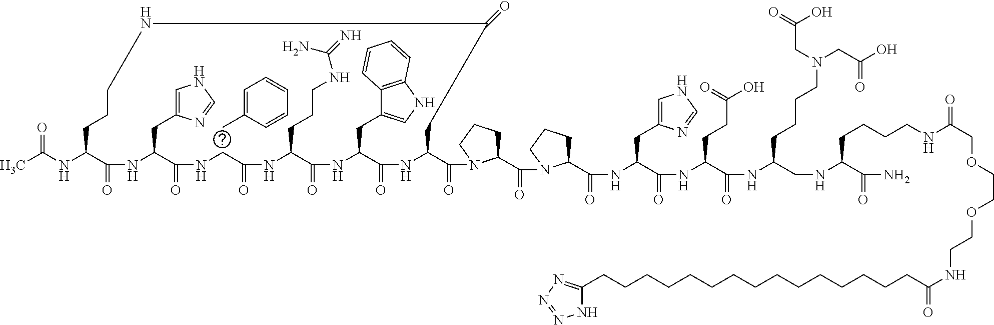 | 49 | 62 |
| 18 | Ac-c[Glu-His-D-Phe- Arg-Trp-Orn]-Pro- Pro-Arg-Asp-Lys (biscarboxymethyl)- Lys({2-[2-(15- carboxypenta- decanoylamino) ethoxy]ethoxy} acetyl)-NH2 | 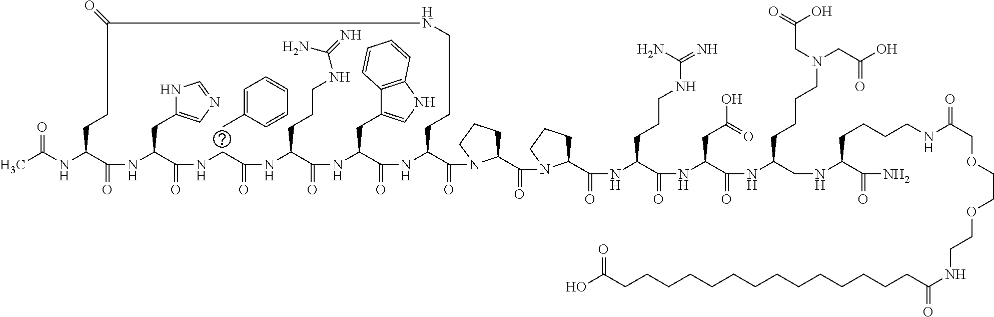 | 57 | 75 |
| 19 | Ac-c[Orn-His-D- Phe-Arg-Trp- Asp]-Pro- Pro-Arg-Glu-Lys (biscarboxymethyl)- Lys[(2-{2- [16-(tetrazol-5-yl) hexadecanoylamino] ethoxy}ethoxy) acetyl]-NH2 | 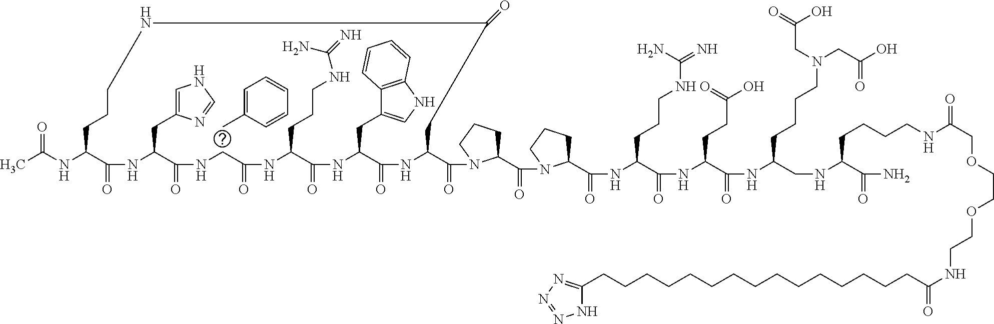 | 51 | 59 |
| 26 | Ac-c[Glu-His-D-Phe- Arg-Trp-Orn]-Pro- Pro-Asp-Lys (biscarboxymethyl)- Lys({2-[2-(17- carboxyhepta- decanoylamino) ethoxy] ethoxy}acetyl)- NH2 | 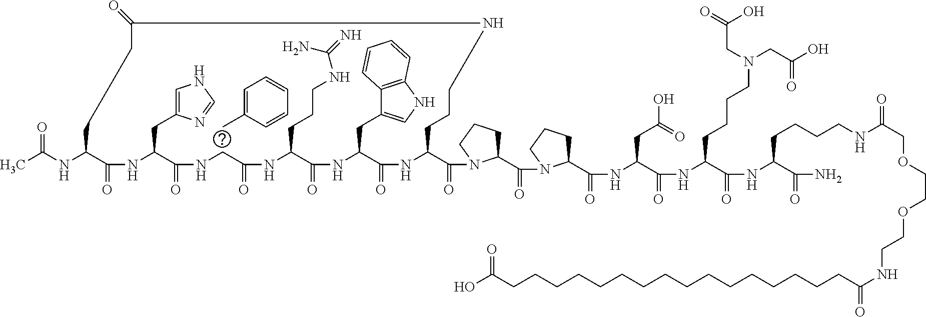 | 58 | 71 |
| 28 | Ac-c[Orn-His-D-Phe- Arg-Trp-Asp]-Pro- Ser-Ser-Ser-Lys (biscarboxymethyl)- Ser-Ser-Lys[(2- {2-[16-(tetrazol-5-yl) hexadecanoylamino] ethoxy}ethoxy) acetyl]-NH2 | 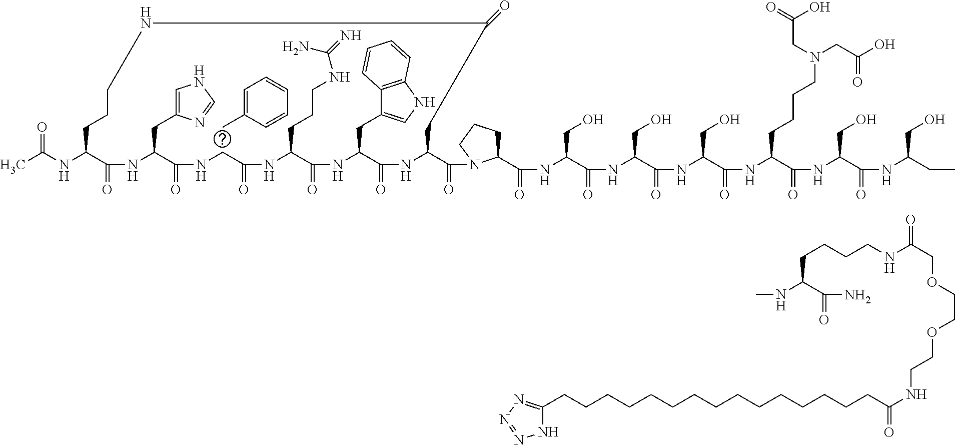 | 40 | 55 |
| 29 | Ac-c[Glu-His-D- Phe-Arg-Trp-Orn]-D- Pro-D-Pro- Arg-Asp-Lys (biscarboxymethyl)- Lys[(2-{2-[16- (tetrazol-5-yl) hexadecanoylamino] ethoxy}ethoxy) acetyl]-NH2 | 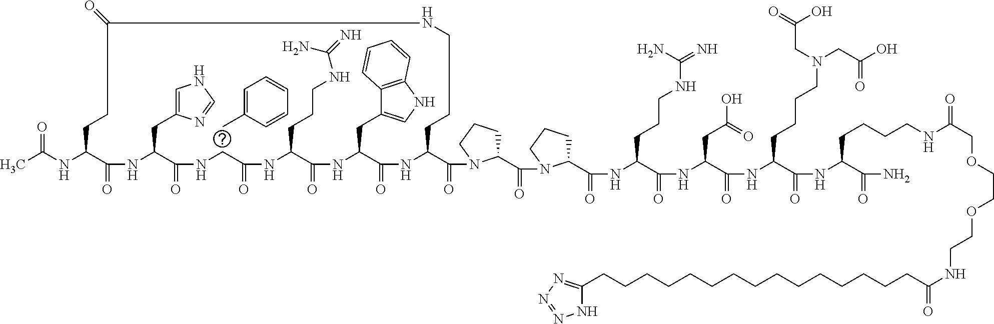 [SMILES: CC(=O)N[C@H]1CCC(=O)NCCC[C@@H](C(=O)N2CCC[C@@H]2C(=O)N2CCC[C@@H]2C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc2nnn[nH]2)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | 54 | 66 |
| 31 | Ac-c[Glu-His-D- Phe-Arg-Trp-Dap]- Hyp-Hyp- Arg-Asp-Lys (biscarboxymethyl)- Lys[(2-{2-[16- (tetrazol-5-yl) hexadecanoylamino] ethoxy}ethoxy) acetyl]-NH2 | 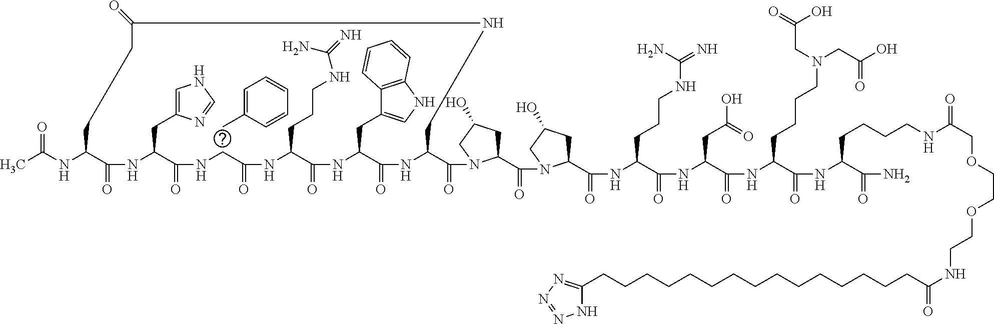 | 89 | 90 |
| 34 | Ac-c[Orn-His-D-Phe- Arg-Trp-Asp]-Pro- Pro-Arg-Glu-Lys (biscarboxymethyl)- Lys({2-[2-(17- carboxyhepta- decanoylamino) ethoxy] ethoxy}acetyl)- NH2 | 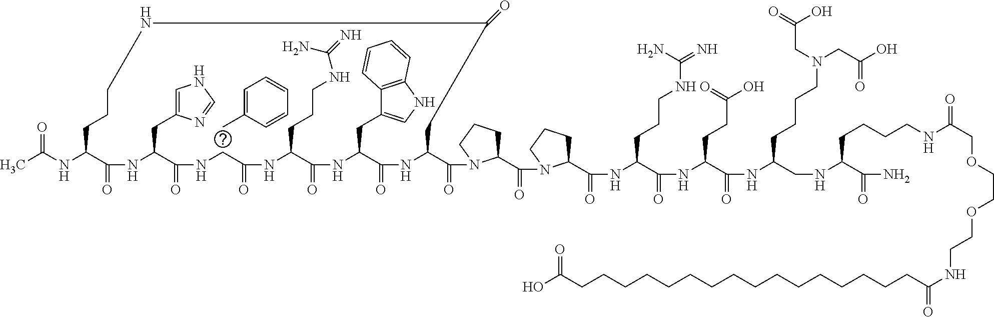 | 43 | 53 |
| 38 | Ac-c[Glu-His-D-Phe- Arg-Trp-Orn]-Pro- Pro-Arg-Asp-Lys (biscarboxymethyl)- Lys({2-[2-(17- carboxyhepta- decanoylamino) ethoxy] ethoxy}acetyl)- NH2 | 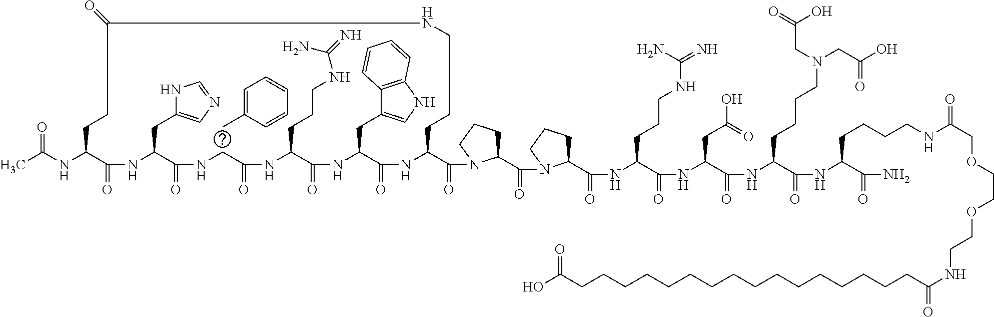 | 58 | 71 |
| 42 | Ac-c[Orn-His-D-Phe- Arg-Trp-Asp]-Pro- Pro-Arg-Glu-Lys (biscarboxymethyl)- Lys({2-[2-(15- carboxypenta- decanoylamino) ethoxy] ethoxy}acetyl)- NH2 | 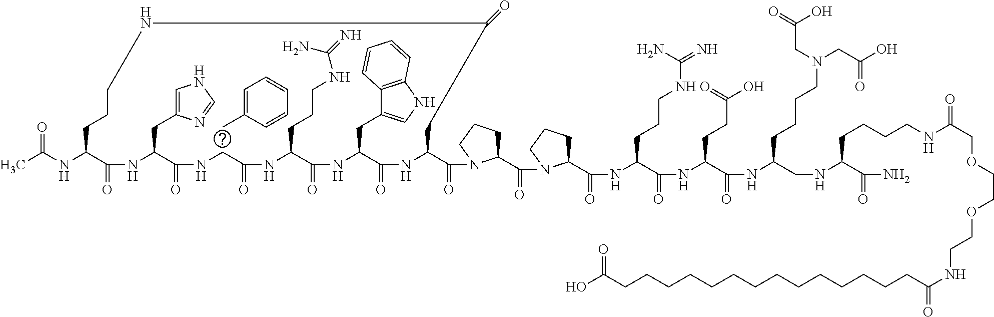 | 44 | 62 |
| 43 | Ac-c[Glu-His-D-Phe- Arg-Trp-Orn]-Pro- Pro-His-Asp-Lys (biscarboxymethyl)- Lys[(2-{2-[16- (tetrazol-5-yl) hexadecanoylamino] ethoxy}ethoxy) acetyl]-NH2 | 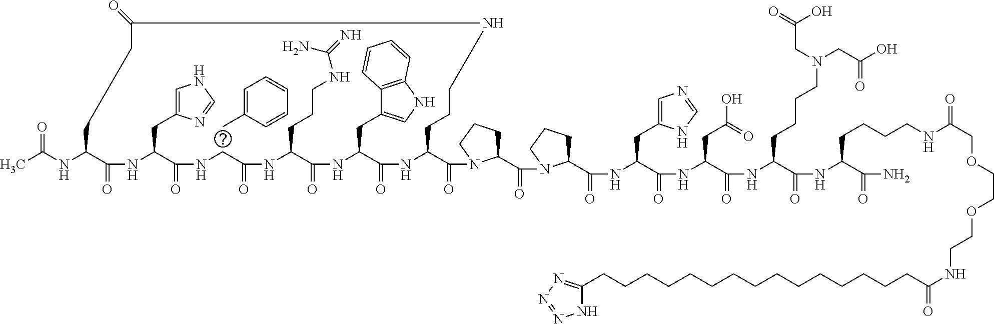 | 49 | 63 |
| 44 | Ac-c[Lys-His-D-Phe- Arg-Trp-Asp]-Pro- Pro-His-Glu-Lys (biscarboxymethyl)- Lys({2-[2-(16- carboxyhexa- decanoylamino) ethoxy] ethoxy}acetyl)- NH2 | 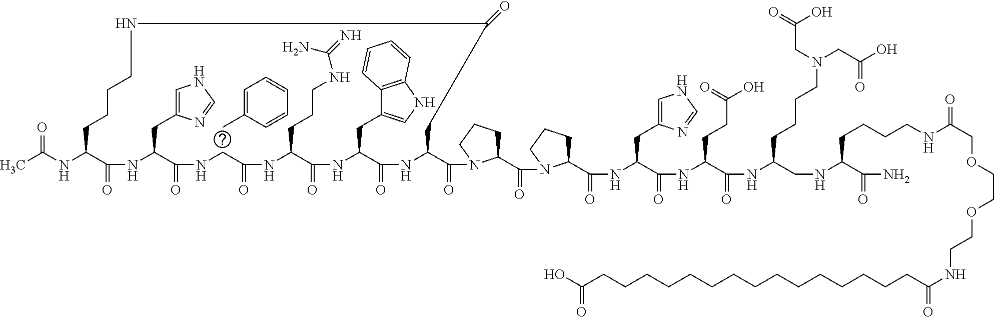 | 68 | 81 |
| 45 | Ac-c[Lys-His-D-Phe- Arg-Trp-Asp]-Pro- Pro-His-Ser-Lys (biscarboxymethyl)- Ser-Ser-Glu- Lys({2-[2-(16- carboxyhexa- decanoylamino) ethoxy] ethoxy}acetyl)- NH2 | 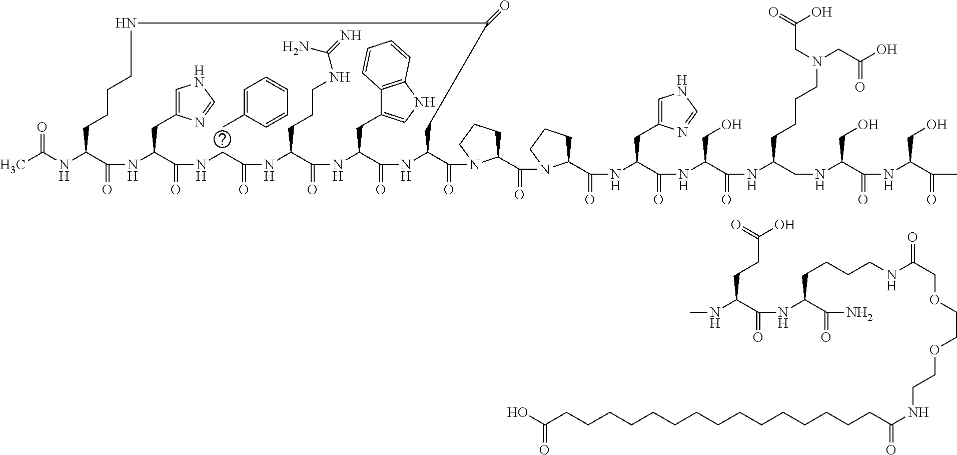 | 68 | 81 |
| 46 | Ac-c[Orn-His-D-Phe- Arg-Trp-Asp]-Pro- Pro-His-Glu-Lys (biscarboxymethyl)- Lys({2-[2-(17- carboxyhepta- decanoylamino) ethoxy] ethoxy}acetyl)- NH2 | 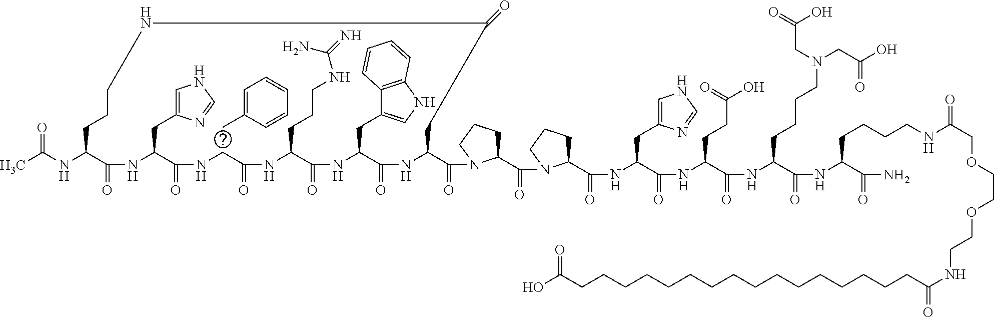 | 44 | 61 |
| 47 | Ac-c[Dap-His-D-Phe- Arg-Trp-Glu]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 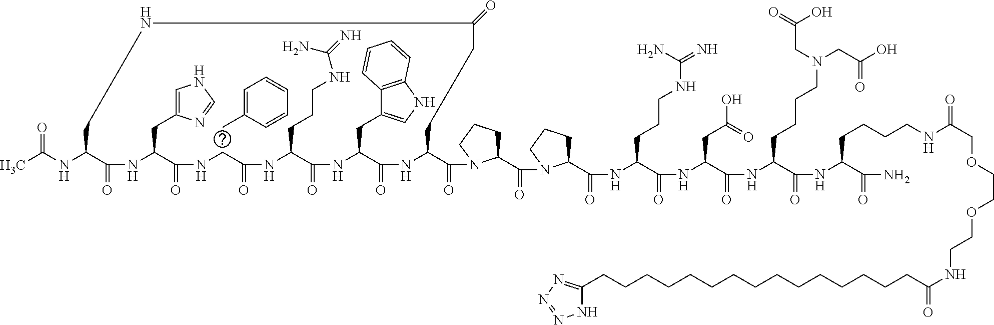 | 35 | 51 |
| 50 | Ac-c[Orn-His-D-Phe- Arg-Trp-Asp]-Pro- Pro-Arg-Glu-Lys (biscarboxymethyl)- Ser-Ser-NH (2-{2-[2-(17- carboxyhepta- decanoylamino) ethoxy]ethoxy} ethyl) | 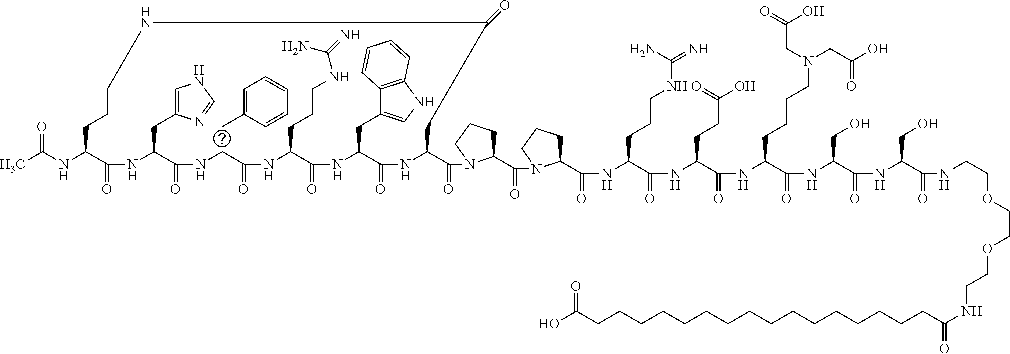 | 53 | 65 |
| 53 | Ac-c[Lys-His-D-Phe- Arg-Trp-Asp]-Pro- Ser-Ser-Ser-Lys (biscarboxymethyl)- Ser-Ser-NH (2-{2-[2-(17- carboxyhepta- decanoylamino) ethoxy] ethoxy}ethyl) | 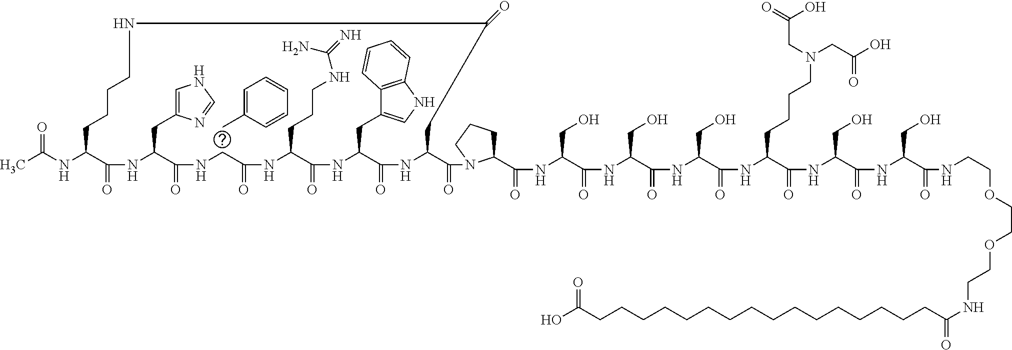 | 59 | 74 |
| 54 | Ac-c[Glu-His-D-Phe- Arg-Trp-Orn]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 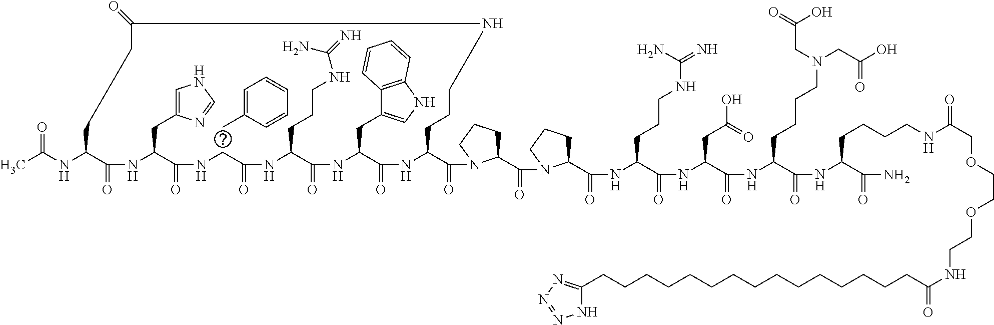 | 42 | 60 |
| 55 | Ac-c[Orn-His-D-Phe- Arg-Trp-Asp]-Pro- Pro-His-Glu-Lys (biscarboxymethyl)- Lys({2-[2-(16- carboxyhexa- decanoylamino) ethoxy] ethoxy}acetyl)- NH2 | 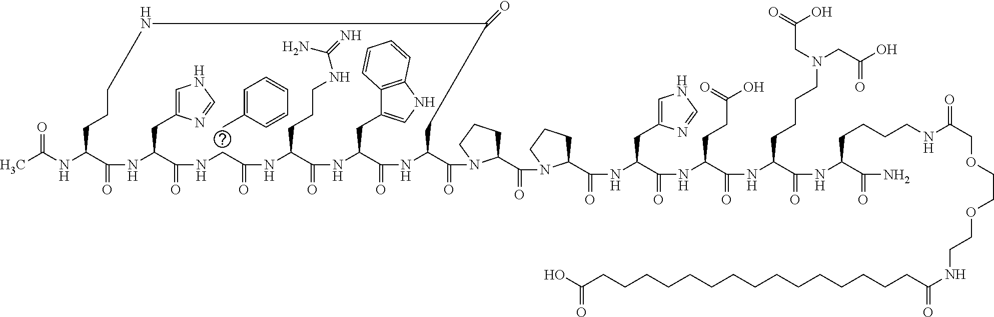 | 46 | 61 |
| 56 | Ac-c[Glu-His-D-Phe- Arg-Trp-Orn]-Hyp- Hyp-Arg-Asp-Lys (biscarboxymethyl)- Lys[(2-{2- [16-(tetrazol-5-yl) hexadecanoylamino] ethoxy}ethoxy) acetyl]-NH2 | 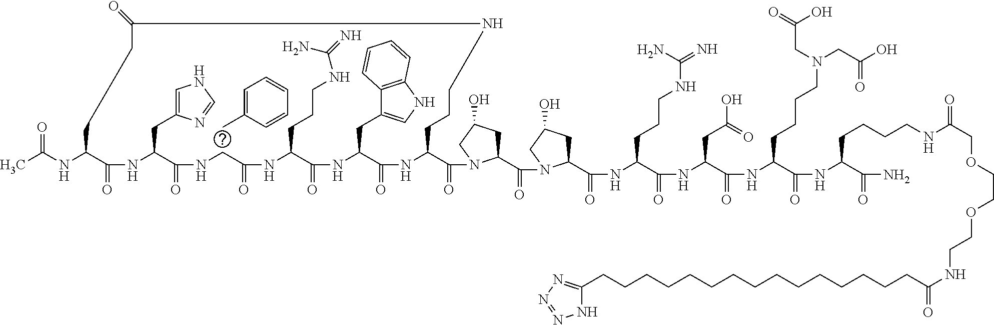 | 58 | 68 |

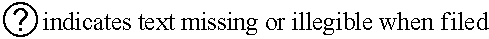

Assay (II)—Melanocortin Receptor 3 and 5 (MC3 and MC5) cAMP Functional Assay Using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer (5 mM HEPES, 0.1% ovalbumin, 0.005% Tween™ 20 and 0.5 mM IBMX, pH 7.4), and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited by increasing the amount of potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the MC4 receptor are stimulated with potential MC4 agonists, and the degree of stimulation of cAMP is measured using the Flash Plate® cAMP assay (NEN™ Life Science Products, cat. No. SMP004).

The MC4 receptor-expressing BHK cells are produced by transfecting the cDNA encoding MC4 receptor into BHK570/KZ10-20-48, and selecting for stable clones expressing the MC4 receptor. The MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 2×10⁶ cells/ml (consumption thereof: 7 ml per 96-well microtiter plate). 50 μl of cell suspension is added to the Flash Plate containing 50 μl of test compound or reference compound (all diluted in PBS, 0.1% HSA and 0.005% Tween). The mixture is shaken for 5 minutes and then allowed to stand for 25 minutes at RT. The reaction is stopped by addition of 100 μl Detection Mix per well (Detection Mix 11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). The assay procedure and the buffers are generally as described in the Flash Plate kit-protocol (Flash Plate® cAMP assay (NEN™ Life Science Products, cat. No. SMP004)). However the cAMP standards are diluted in PBS with 0.1% HSA and 0.005% Tween™ 20 and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay was performed on BHK cell membranes stably expressing the MC1 receptor. The assay was performed in a total volume of 250 μl: 25 μl of ¹²⁵NDP-α-MSH (22 pM in final concentration), 25 μl of test compound/control and 200 μl of cell membrane (25 μg/ml). Test compounds were dissolved in DMSO. Radioactively labeled ligand, membranes and test compounds were diluted in buffer: 25 mM HEPES, pH 7.4, 0.1 mM CaCl₂, 1 mM MgSO₄, 1 mM EDTA, 0.1% HSA and 0.005% Tween™ 20. Alternatively, HSA may be substituted with ovalbumin. The samples were incubated at 30° C. for 90 min. in Costar round-bottom microtiter plates. Incubation was terminated by filtration on a Packard harvester filtermate. Rapid filtration through Packard Unifilter-96 GF/B filters pre-treated with polyetylenimine (PerkinElmer 6005277). The filters were washed with ice-cold 0.9% NaCl 8-10 times. The plates were air-dried at app. 55° C. for 30 min, and 50 μl Microscint 0 (Packard, cat. No. 6013616) was added to each well. The plates were counted in a Topcounter (1 min/well).

The data were analysed by non-linear regression analysis of binding curves, using the Windows™ program GraphPad™ Prism (GraphPad Software, USA).

TABLE 3
In vitro data on receptor binding

| | | | Assay | Assay |
|---|---|---|---|---|
| Ex- | | | (V) | (IV) |
| am- | | | MC4 | MC1 |
| ple | | | [Ki] | [Ki] |
| nr. | Compound | Molecule | (nM) | (nM) |
| 1 | Ac-c[Dap- His-D-Phe- Arg-Trp- Asp]-Pro-Pro- Arg-Asp-Lys{[bis (carboxymethyl) amino]acetyl}- Lys[(2-{2- [16-(tetrazol-5-yl) hexadecanoylamino] ethoxy}ethoxy) acetyl]-NH2 | 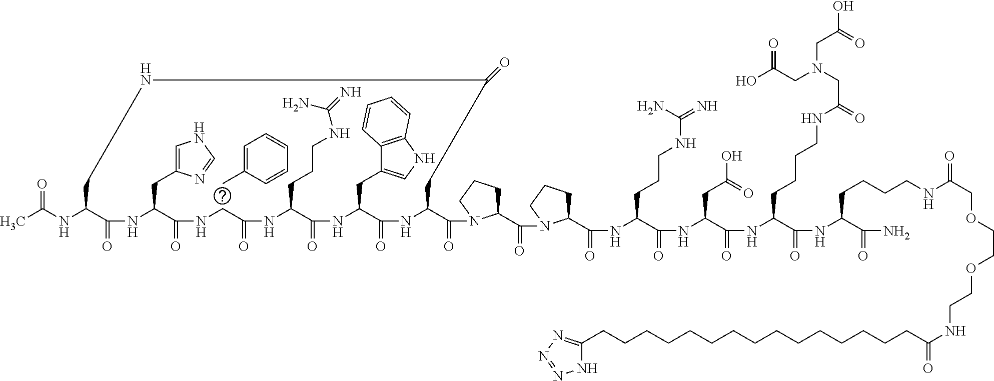 | 8.95 | 5549 |
| 2 | Ac-c[Asp- His-D-Phe- Arg-Trp-Orn]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 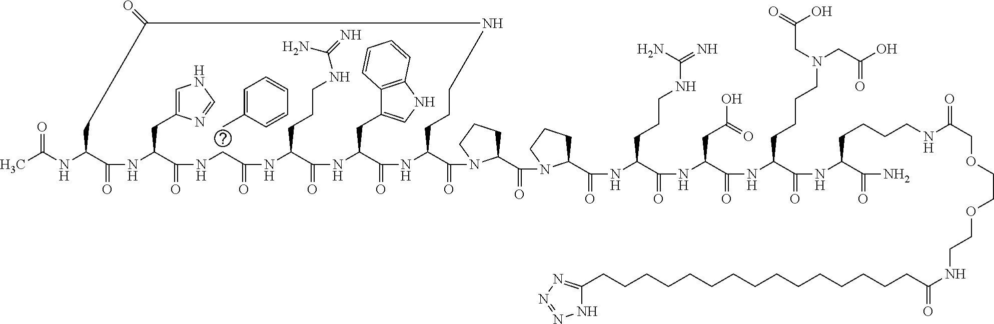 | 33 | 2539 |
| 3 | Ac-c[Dab- His-D-Phe- Arg-Trp-Asp]-Pro- Ser-Ser-Ser-Lys (biscarboxymethyl)- Ser-Ser-NH (2-{2-[2-(17- carboxyhepta- decanoylamino) ethoxy] ethoxy}ethyl) | 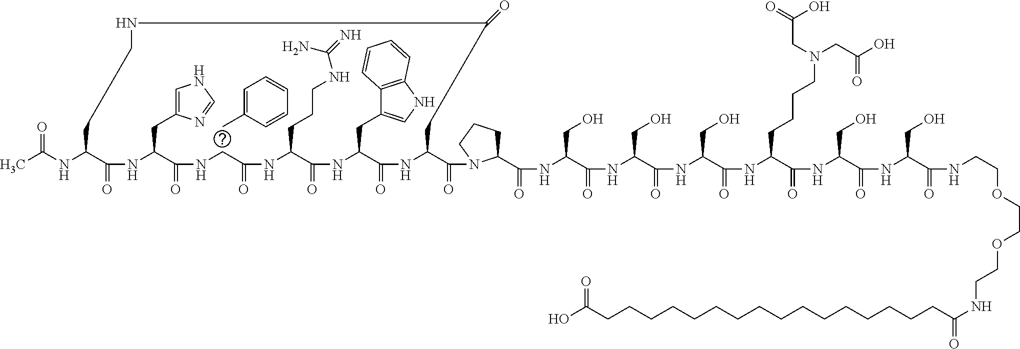 | 10.8 | 9130 |
| 4 | Ac- c[homoCys-His- D-Phe-Arg- Trp-Cys]- Pro-Pro- Lys-Asp- Dap{[bis (carboxymethyl) amino]acetyl}- Lys[(2-{2- [16-(tetrazol-5-yl) hexadecanoylamino] ethoxy}ethoxy) acetyl]-OH | 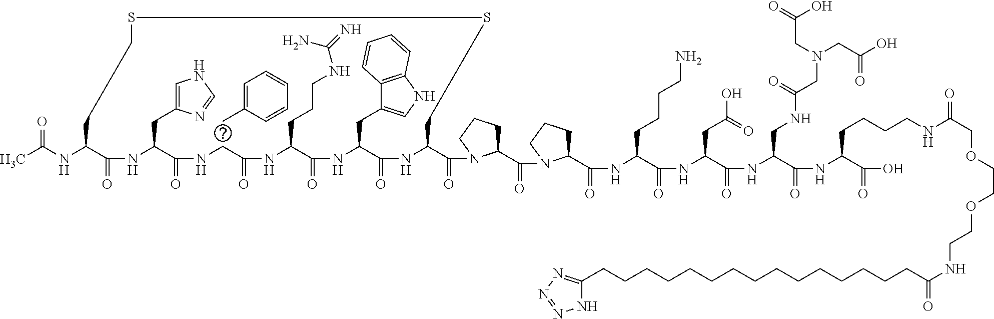 | 6.4 | >10000 |
| 5 | Ac- c[homoCys-His-D- Phe-Arg-Trp-Cys]- Pro-Pro-Lys- Glu-Lys (biscarboxymethyl)- Lys({2-[2-(15- carboxypenta- decanoylamino) ethoxy] ethoxy}acetyl)- NH2 | 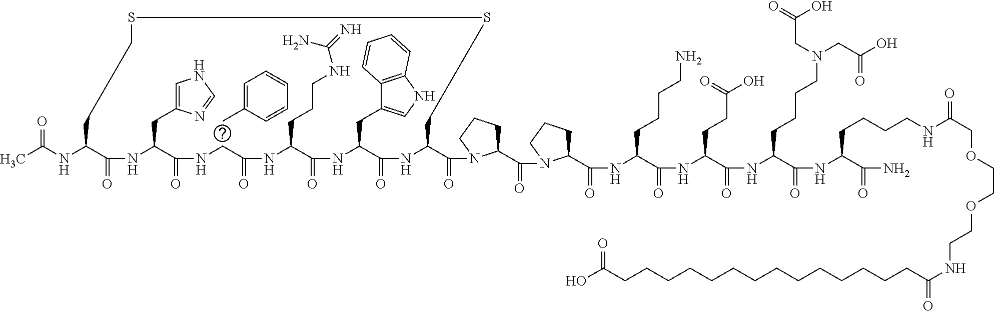 | 17 | >10000 |
| 6 | Ac-c[Glu- His-D-Phe- Arg-Trp- Dap]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 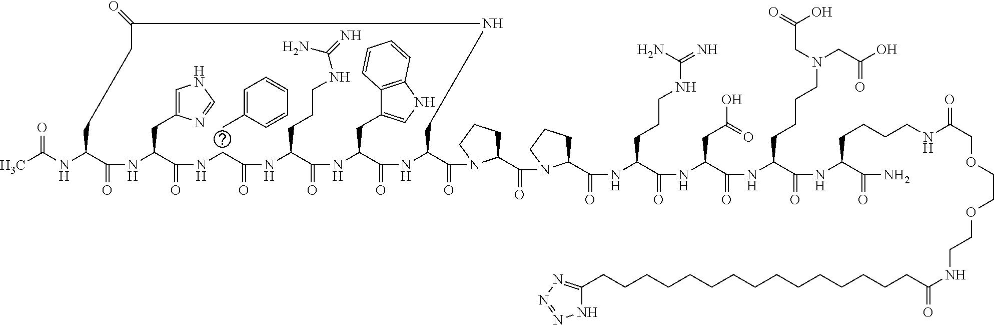 | 13 | >10000 |
| 7 | Ac-c[Lys- His-D-Phe- Arg-Trp- Glu]-Pro- Pro-His-Glu-Lys (biscarboxymethyl)- Lys({2-[2-(16- carboxyhexa- decanoylamino) ethoxy]ethoxy} acetyl)-NH2 | 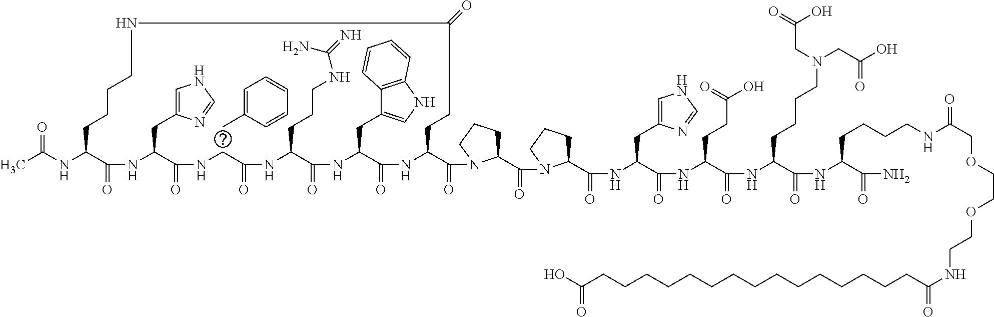 | 348 | >10000 |
| 8 | Ac-c[Orn- His-D-Phe- Arg-Trp- Glu]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 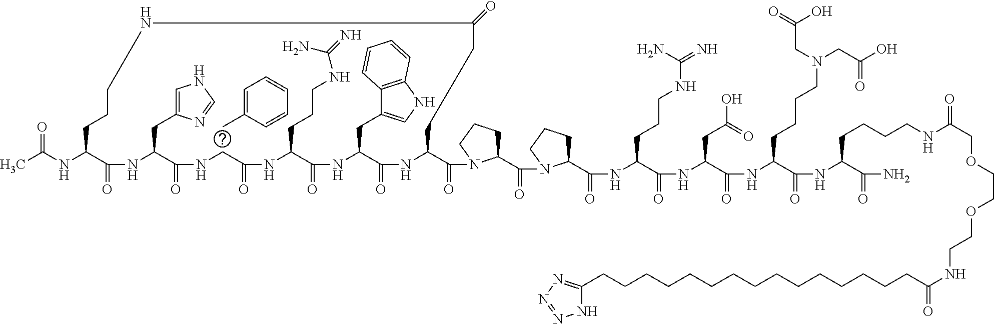 | 3.53 | 433 |
| 9 | Ac-c[Dab- His-D-Phe- Arg-Trp-Asp]-Pro- Pro-Arg-Glu-Lys (biscarboxymethyl)- Ser-Ser-NH(2- {2-[2-(17- carboxyhepta- decanoylamino) ethoxy]ethoxy} ethyl) | 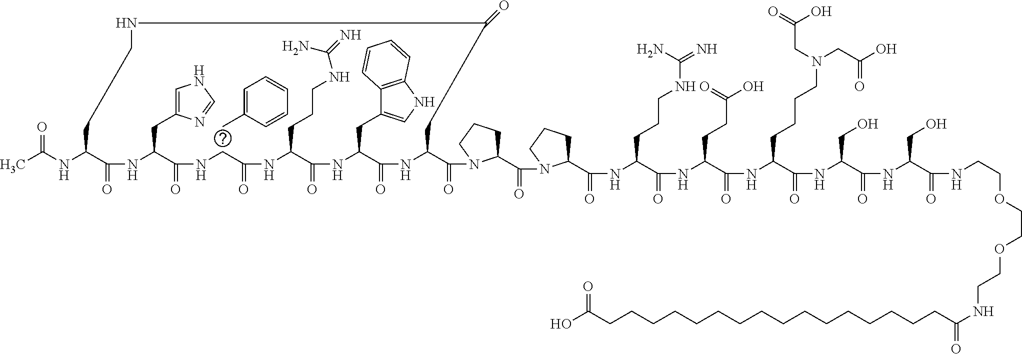 | 12.5 | >10000 |
| 10 | Ac-c[Orn- His-D-Phe- Arg-Trp-Asp]-Pro- Pro-His-Glu-Lys (biscarboxymethyl)- Lys[(2-{2- [16-(tetrazol-5-yl) hexadecanoylamino] ethoxy}ethoxy) acetyl]-NH2 | 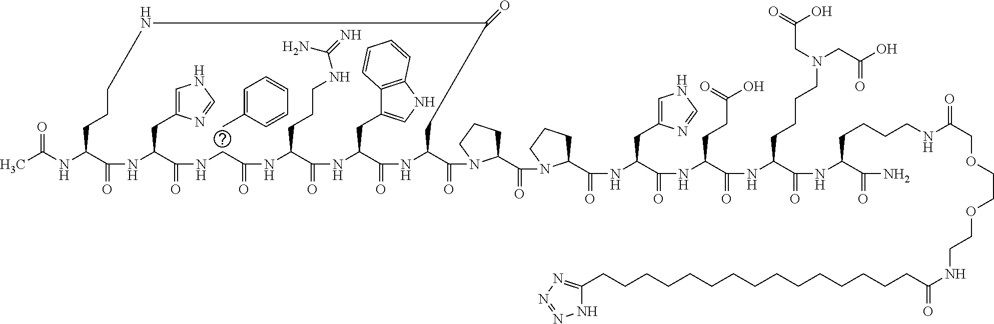 | 1.43 | >10000 |
| 11 | Ac-c[Lys- His-D-Phe- Arg-Trp- Asp]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 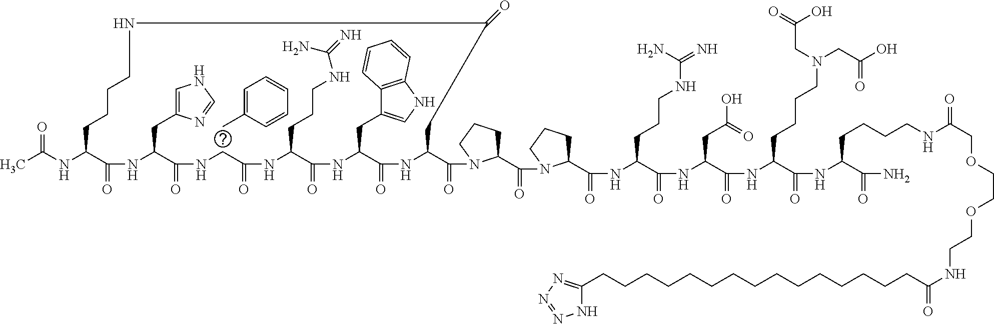 | 3.87 | >10000 |
| 12 | Ac-c[Orn- His-D-Phe- Arg-Trp-Asp]-Pro- Ser-Ser-Ser-Lys (biscarboxymethyl)- Ser-Ser-Lys ({2-[2-(15- carboxypenta- decanoylamino) ethoxy]ethoxy} acetyl)-NH2 | 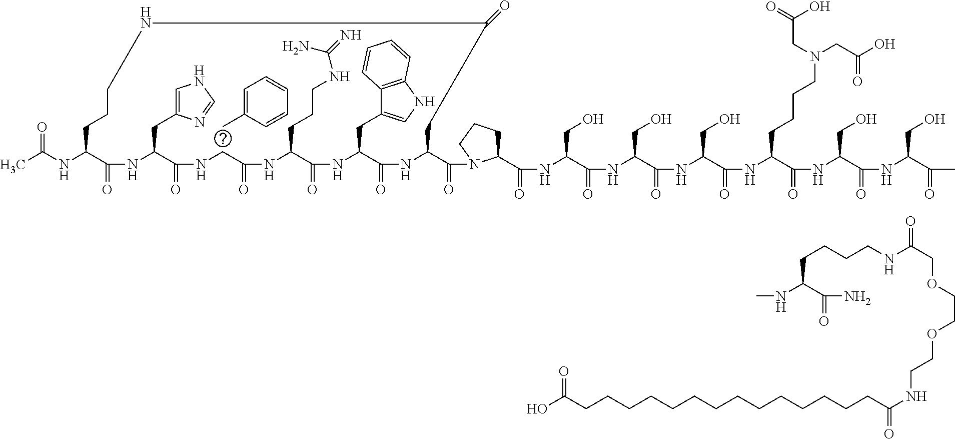 | 1.2 | 3888 |
| 13 | Ac-c[Glu- His-D-Phe- Arg-Trp-Orn]-Pro- Pro-Asp-Lys (biscarboxymethyl)- Lys({2-[2-(15- carboxypenta- decanoylamino) ethoxy]ethoxy} acetyl)-NH2 | 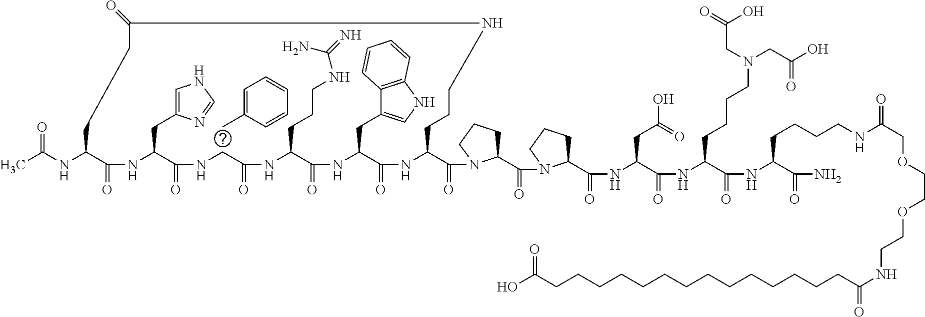 | 64 | >10000 |
| 14 | Ac-c[Orn- His-D-Phe- Arg-Trp-Asp]-Pro- Ser-Ser-Ser-Lys (biscarboxymethyl)- Ser-Ser-Lys ({2-[2-(17- carboxyhepta- decanoylamino) ethoxy]ethoxy} acetyl)-NH2 | 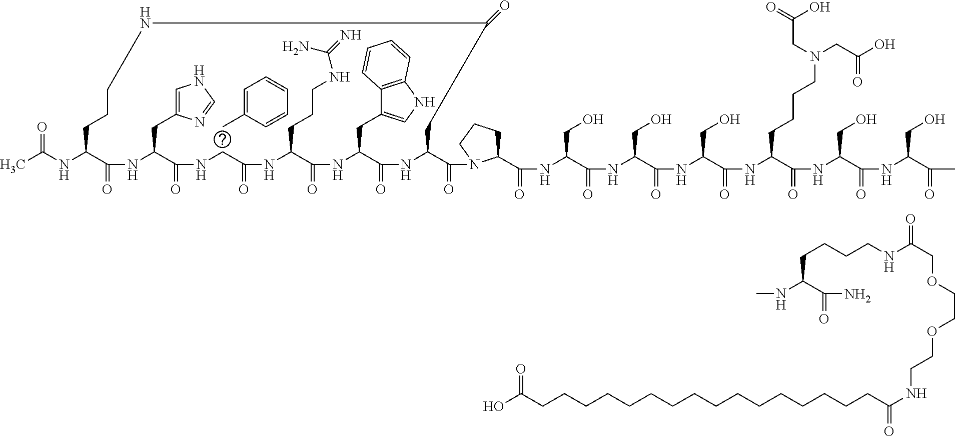 | 0.8 | 141 |
| 15 | Ac-c[Dab- His-D-Phe- Arg-Trp-Asp]-Pro- Pro-Arg-Glu-Lys (biscarboxymethyl)- Lys[(2-{2- [16-(tetrazol-5-yl) hexadecanoylamino] ethoxy}ethoxy) acetyl]-NH2 | 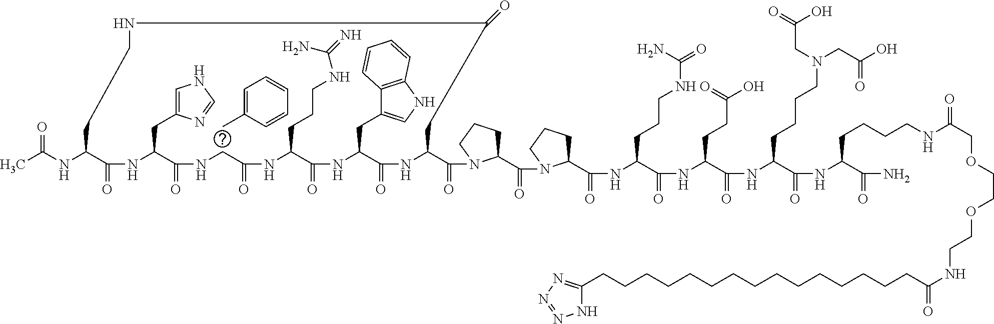 | 8.45 | >10000 |
| 16 | Ac-c[Glu- His-D-Phe- Arg-Trp-Dab]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 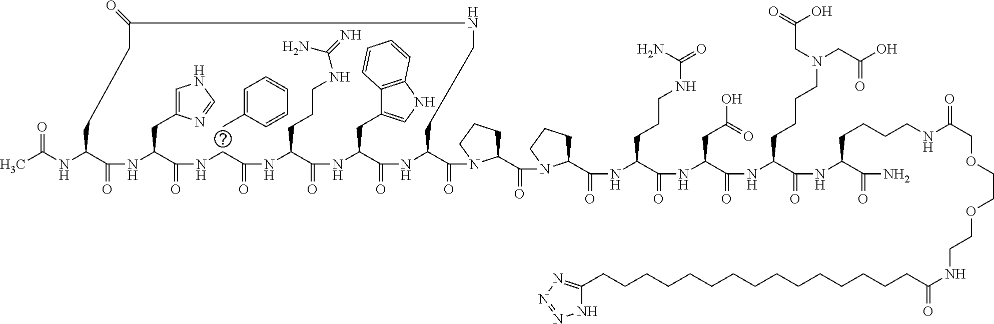 | 39 | 3422 |
| 17 | Ac-c[Orn- His-D-Phe- Arg-Trp-Asp]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 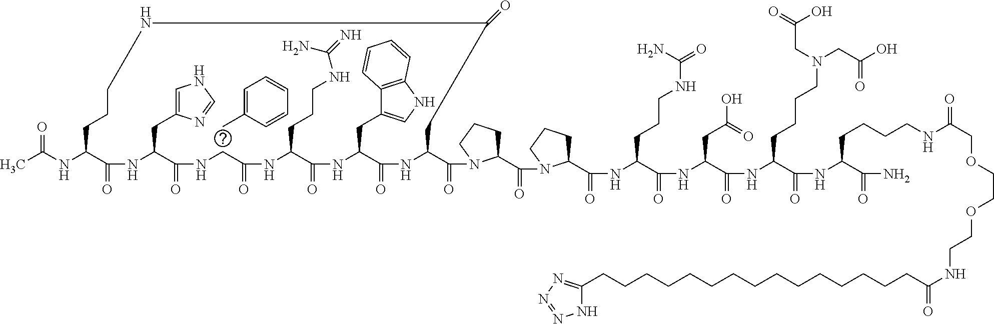 | 0.85 | 2340 |
| 18 | Ac-c[Glu- His-D-Phe- Arg-Trp-Orn]-Pro- Pro-Arg-Asp-Lys (biscarboxymethyl)- Lys({2-[2-(15- carboxypenta- decanoylamino) ethoxy]ethoxy} acetyl)-NH2 | 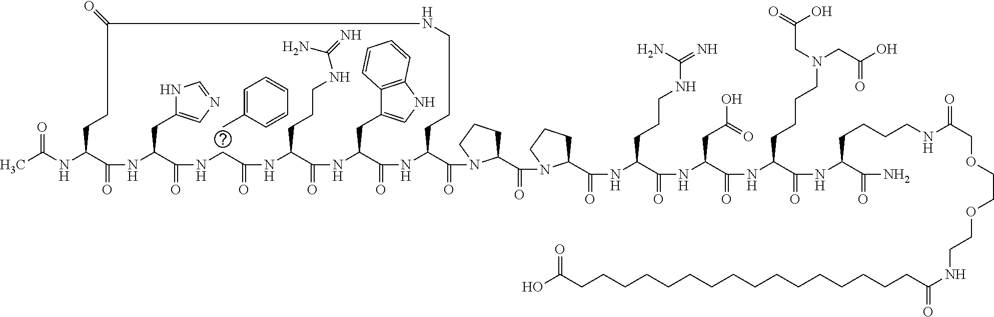 | 5.8 | 3972 |
| 19 | Ac-c[Orn- His-D-Phe- Arg-Trp-Asp]-Pro- Pro-Arg-Glu-Lys (biscarboxymethyl)- Lys[(2-{2- [16-(tetrazol-5-yl) hexadecanoylamino] ethoxy}ethoxy) acetyl]-NH2 | 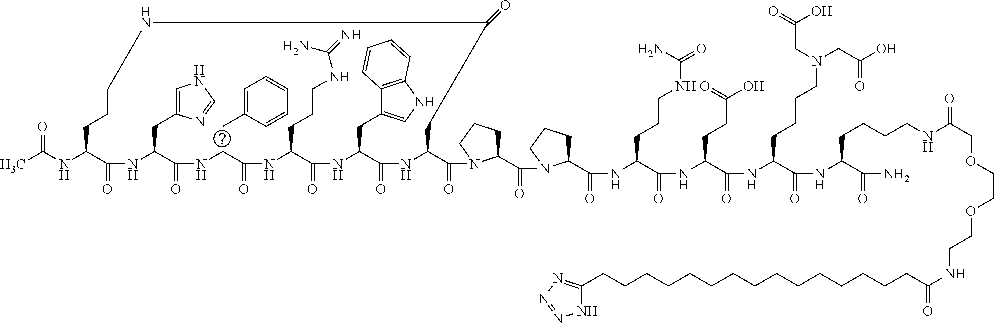 | 0.33 | 2773 |
| 20 | Ac-c[Orn- His-D-Phe- Arg-Trp- Glu]-Pro- Ser-Lys-Glu- Glu-Ser- Ser-Glu- Lys({2-[2-(16- carboxyhexa- decanoylamino) ethoxy] ethoxy}acetyl)- NH2 | 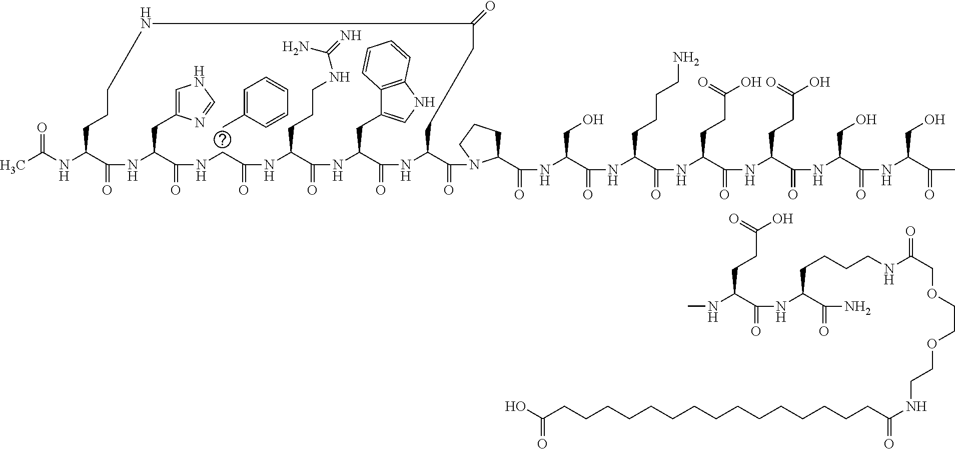 | 38 | >10000 |
| 21 | Ac-c[Lys- His-D-Phe- Arg-Trp-Glu]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 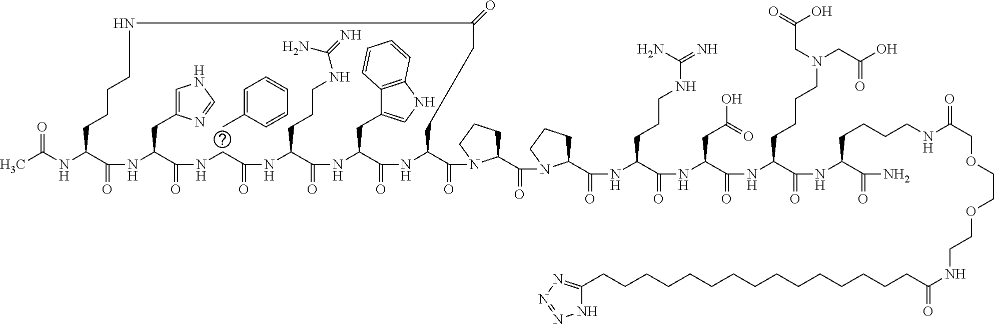 | 179 | >10000 |
| 22 | Ac-c[Orn- His-D-Phe- Arg-Trp-Glu]-Pro- Pro-His-Glu-Lys (biscarboxymethyl)- Lys({2-[2-(16- carboxyhexa- decanoylamino) ethoxy]ethoxy} acetyl)-NH2 | 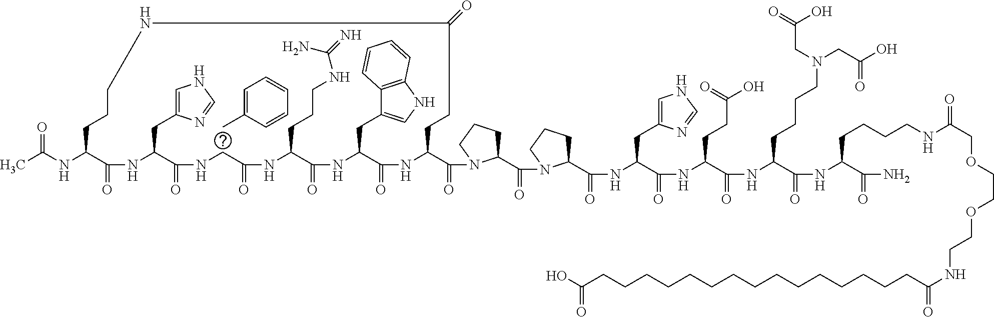 | 21.5 | >10000 |
| 23 | Ac-c[Dab- His-D-Phe- Arg-Trp-Glu]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 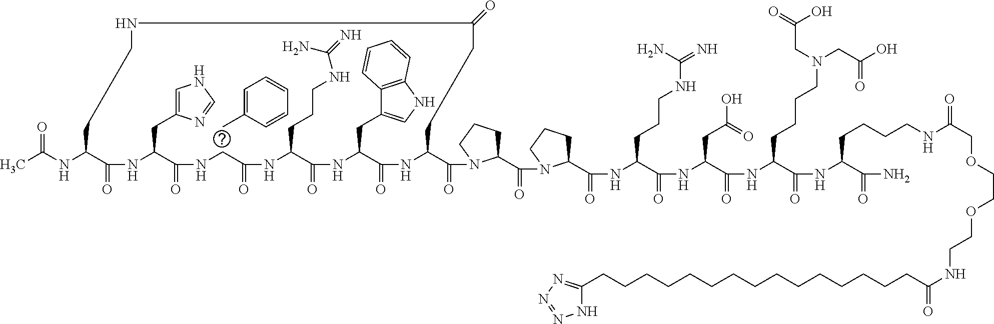 | 107 | >10000 |
| 24 | Ac-c[Asp- His-D-Phe- Arg-Trp-Dap]-Pro- Pro-Arg-Asp- Lys{[bis (carboxymethyl) amino]acetyl}- Lys[(2-{2- [16-(tetrazol-5-yl) hexadecanoylamino] ethoxy}ethoxy) acetyl]-NH2 | 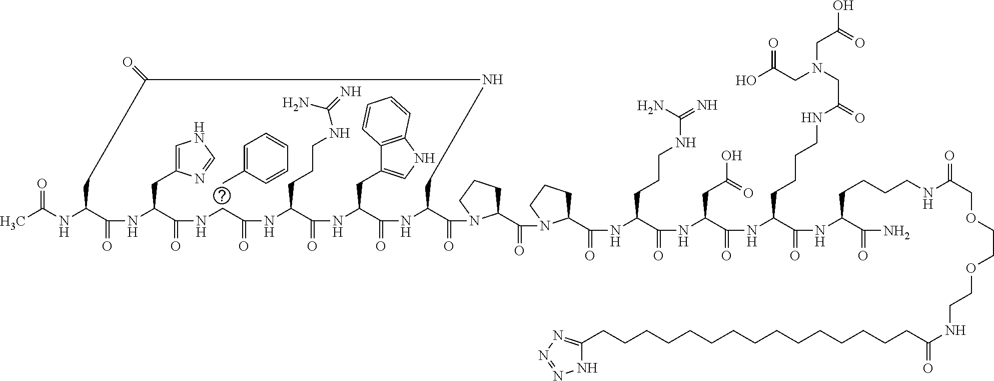 | 107 | 7977 |
| 25 | Ac-c[Dab- His-D-Phe- Arg-Trp-Asp]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 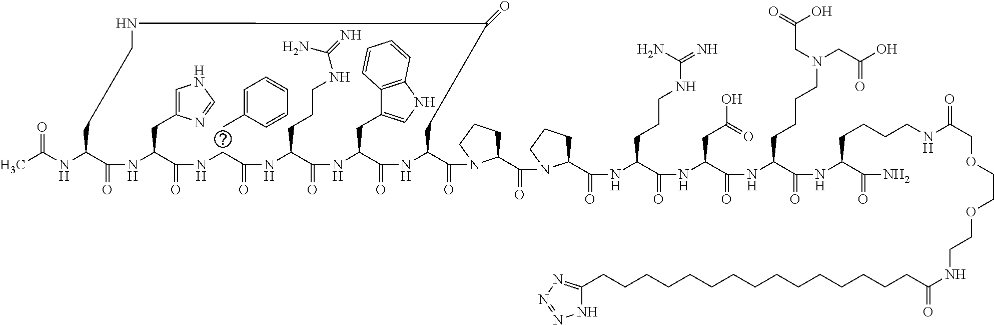 | 31 | 5736 |
| 26 | Ac-c[Glu- His-D-Phe- Arg-Trp-Orn]-Pro- Pro-Asp-Lys (biscarboxymethyl)- Lys({2-[2-(17- carboxyhepta- decanoylamino) ethoxy]ethoxy} acetyl)-NH2 | 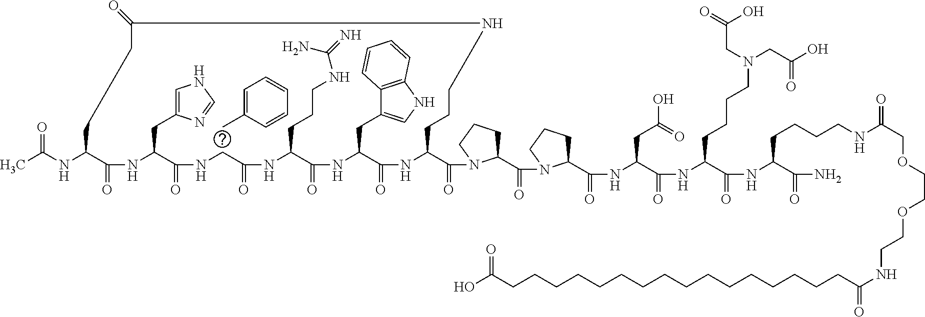 | 8.6 | >10000 |
| 27 | Ac-c[Glu- His-D-Phe- Arg-Trp-Lys]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 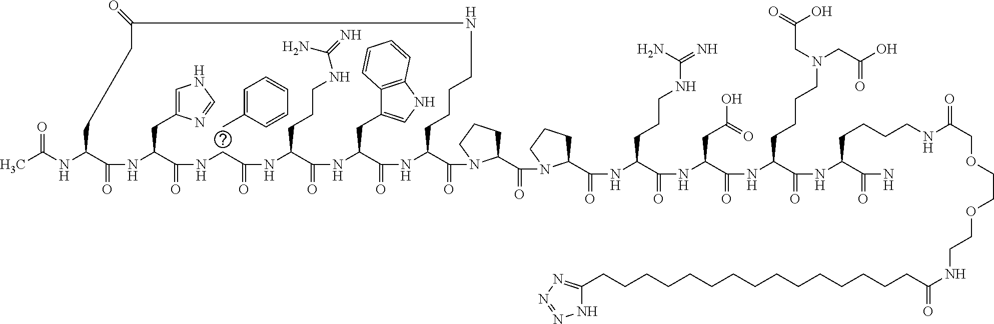 | 24 | >10000 |
| 28 | Ac-c[Orn- His-D-Phe- Arg-Trp-Asp]-Pro- Ser-Ser-Ser-Lys (biscarboxymethyl)- Ser-Ser-Lys[(2- {2-[16- (tetrazol-5-yl) hexadecanoylamino] ethoxy}ethoxy) acetyl]-NH2 | 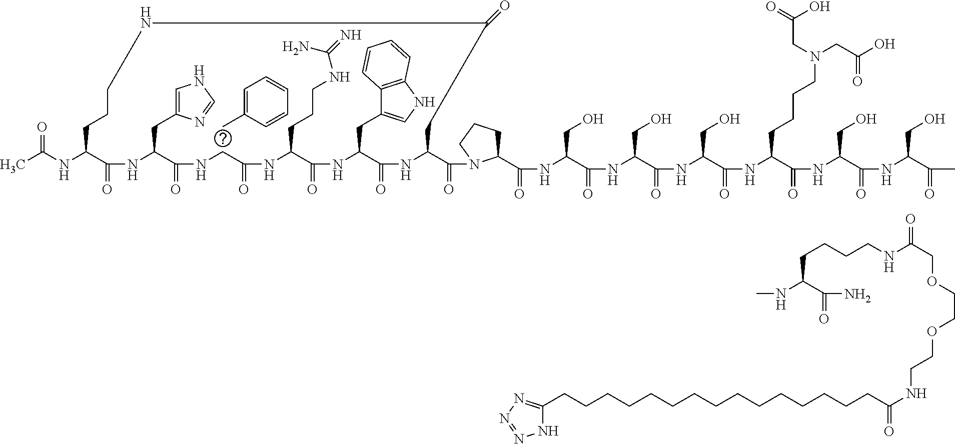 | 0.9 | 5610 |
| 29 | Ac-c[Glu- His-D-Phe- Arg-Trp- Orn]-D-Pro- D-Pro-Arg- Asp-Lys (biscarboxymethyl)- Lys[(2-{2- [16-(tetrazol-5-yl) hexadecanoylamino] ethoxy}ethoxy) acetyl]-NH2 | 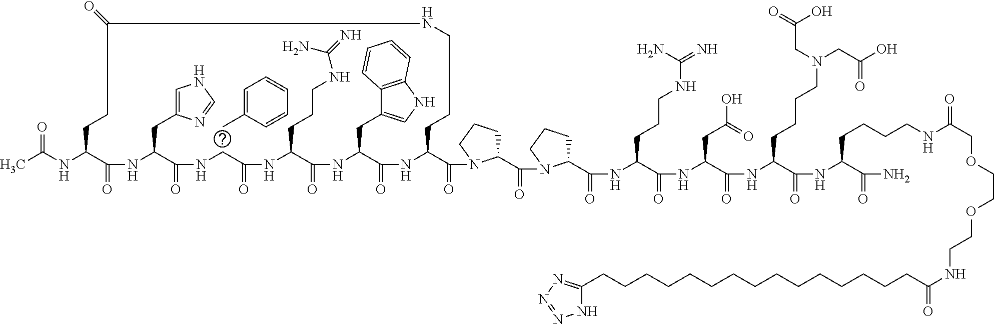 | 4.6 | 8841 |
| 30 | Ac-c[Asp- His-D-Phe- Arg-Trp-Dab]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 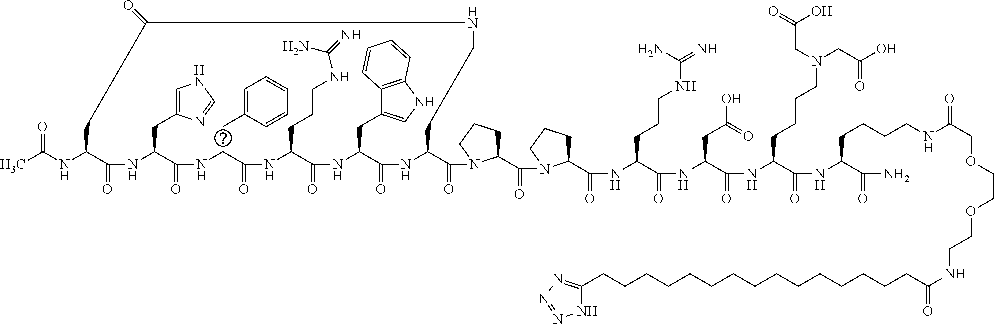 | 1.33 | 5145 |
| 31 | Ac-c[Glu- His-D-Phe- Arg-Trp-Dap]-Hyp- Hyp-Arg-Asp-Lys (biscarboxymethyl)- Lys[(2-{2- [16-(tetrazol-5-yl) hexadecanoylamino] ethoxy}ethoxy) acetyl]-NH2 | 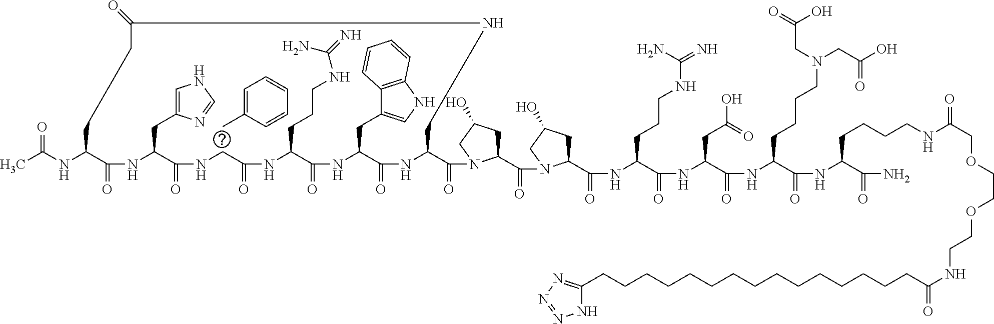 | 17 | >10000 |
| 32 | Ac-c[Glu- His-D-Phe- Arg-Trp-Dab]-Hyp- Hyp-Arg-Asp-Lys (biscarboxymethyl)- Lys[(2-{2- [16-(tetrazol-5-yl) hexadecanoylamino] ethoxy}ethoxy) acetyl]-NH2 | 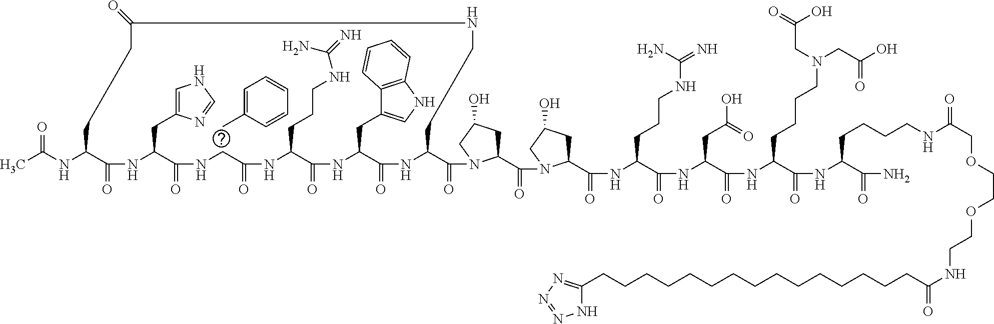 | 105 | >10000 |
| 33 | Ac-c[Dab- His-D-Phe- Arg-Trp-Asp]-Pro- Pro-His-Glu-Lys (biscarboxymethyl)- Lys[(2-{2- [16-(tetrazol-5-yl) hexadecanoylamino] ethoxy}ethoxy) acetyl]-NH2 | 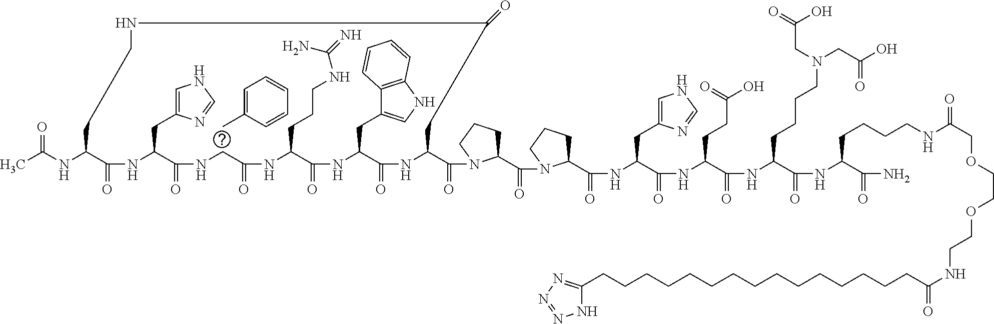 | 76 | >10000 |
| 34 | Ac-c[Orn- His-D-Phe- Arg-Trp-Asp]-Pro- Pro-Arg-Glu-Lys (biscarboxymethyl)- Lys({2-[2-(17- carboxyhepta- decanoylamino) ethoxy]ethoxy} acetyl)-NH2 | 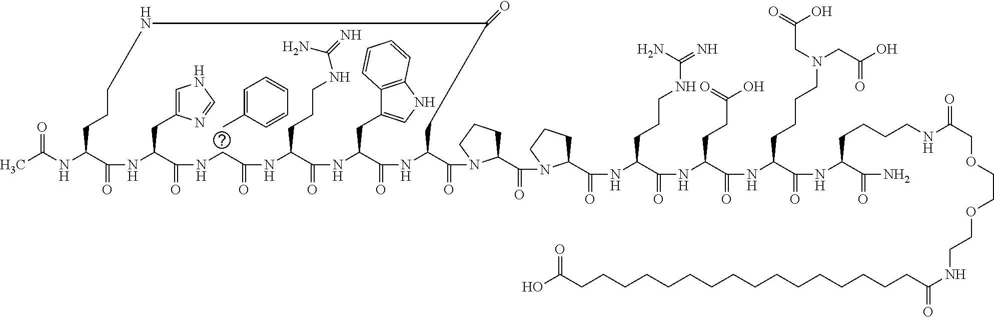 | 0.8 | 1940 |
| 35 | Ac-c[Asp- His-D-Phe- Arg-Trp-Lys]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 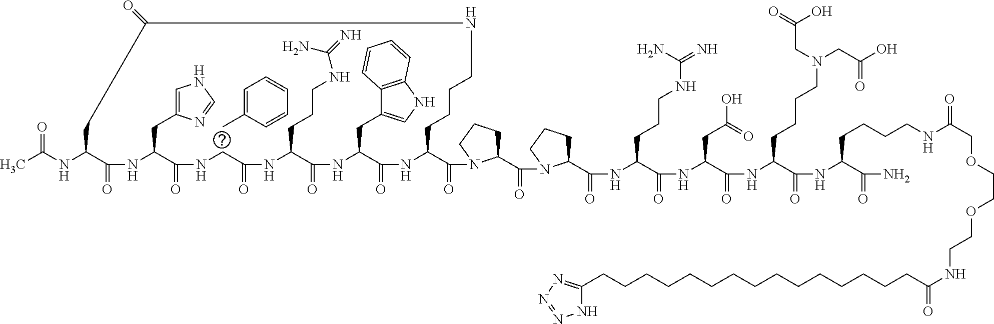 | 13 | 279 |
| 36 | Ac-c[Dap- His-D-Phe- Arg-Trp-Asp]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 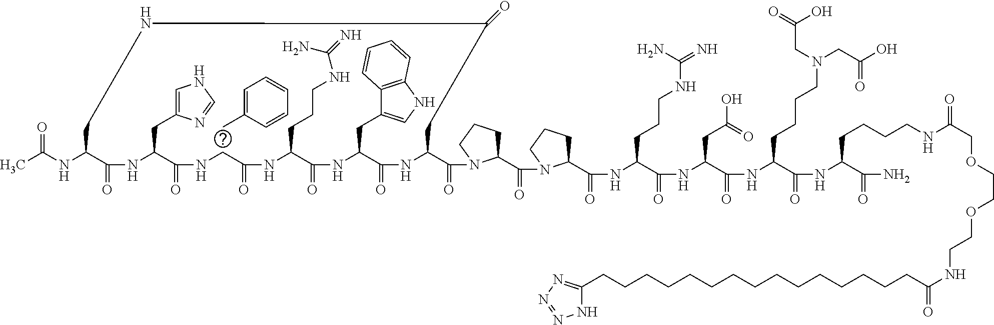 | 5.16 | 1831 |
| 37 | Ac-c[Glu- Gln-D-Phe- Arg-Trp-Orn]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 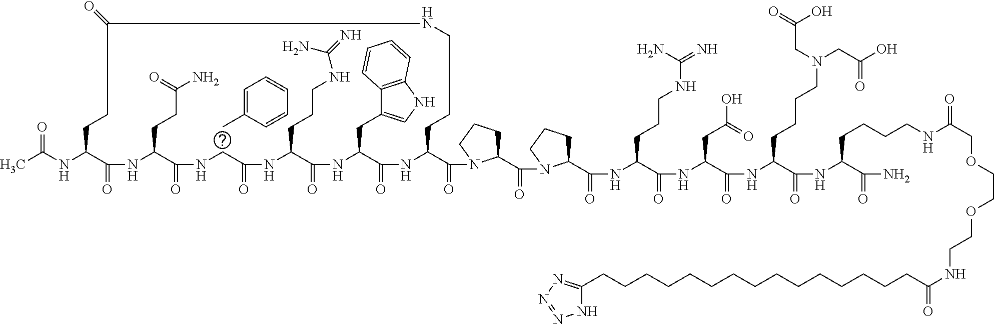 | 58 | >10000 |
| 38 | Ac-c[Glu- His-D-Phe- Arg-Trp-Orn]-Pro- Pro-Arg-Asp-Lys (biscarboxymethyl)- Lys({2-[2-(17- carboxyhepta- decanoylamino) ethoxy]ethoxy} acetyl)-NH2 | 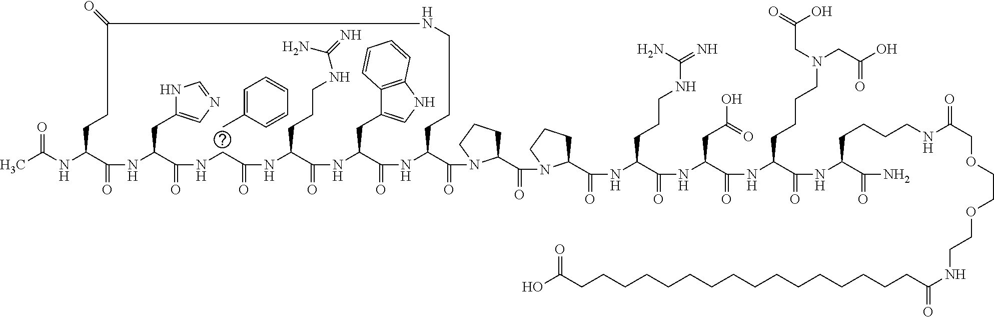 | 0.9 | 4692 |
| 39 | Ac-c[Orn- His-D-Phe- Arg-Trp-Glu]-Pro- Ser-Ser- Ser-Glu-Glu- Ser-Ser-Lys({2-[2- (16-carboxyhexa- decanoylamino) ethoxy]ethoxy} acetyl)-NH2 | 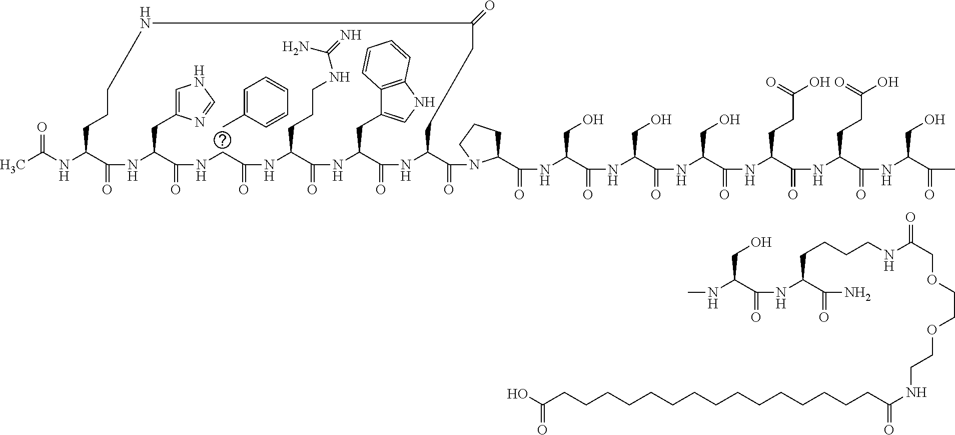 | 25 | >10000 |
| 40 | Ac-c[Orn- Glu-His-D- Phe-Arg-Trp-Gly- Asp]-Pro-Ser-Lys- Glu-Glu-Ser-Ser- Lys({2-[2-(16- carboxyhexa- decanoylamino) ethoxy]ethoxy} acetyl)-NH2 | 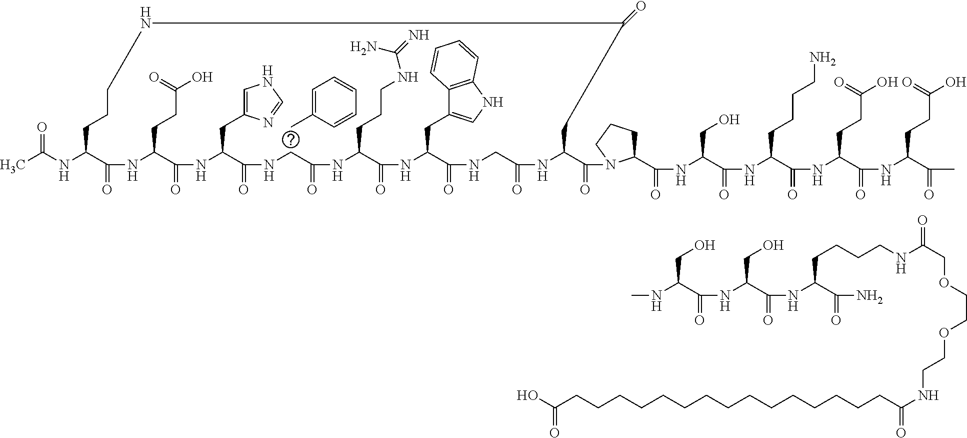 | 125 | >10000 |
| 41 | Ac-c[Asp- His-D-Phe- Arg-Trp-Dap]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 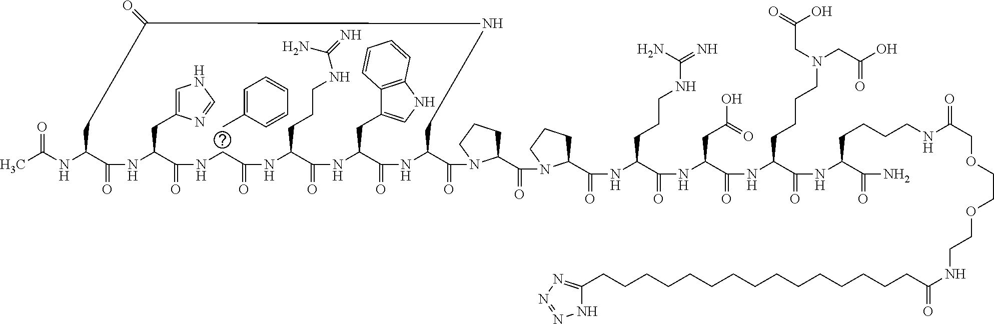 | 71.5 | >10000 |
| 42 | Ac-c[Orn- His-D-Phe- Arg-Trp-Asp]-Pro- Pro-Arg-Glu-Lys (biscarboxymethyl)- Lys({2-[2-(15- carboxypenta- decanoylamino) ethoxy]ethoxy} acetyl)-NH2 | 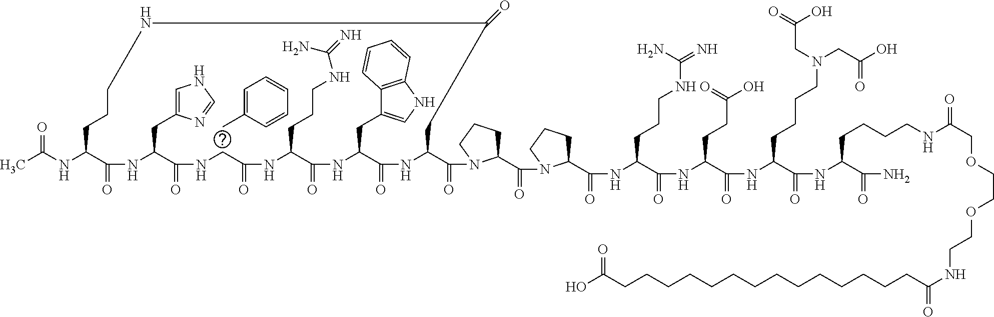 | 2.2 | 2136 |
| 43 | Ac-c[Glu- His-D-Phe- Arg-Trp-Orn]-Pro- Pro-His-Asp-Lys (biscarboxymethyl)- Lys[(2-{2- [16-(tetrazol-5-yl) hexadecanoylamino] ethoxy}ethoxy) acetyl]-NH2 | 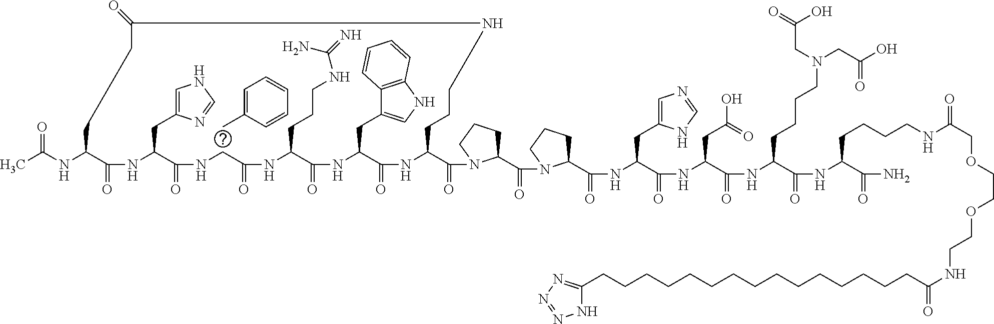 | 3.58 | 5166 |
| 44 | Ac-c[Lys- His-D-Phe- Arg-Trp-Asp]-Pro- Pro-His-Glu-Lys (biscarboxymethyl)- Lys({2-[2-(16- carboxyhexa- decanoylamino) ethoxy]ethoxy} acetyl)-NH2 | 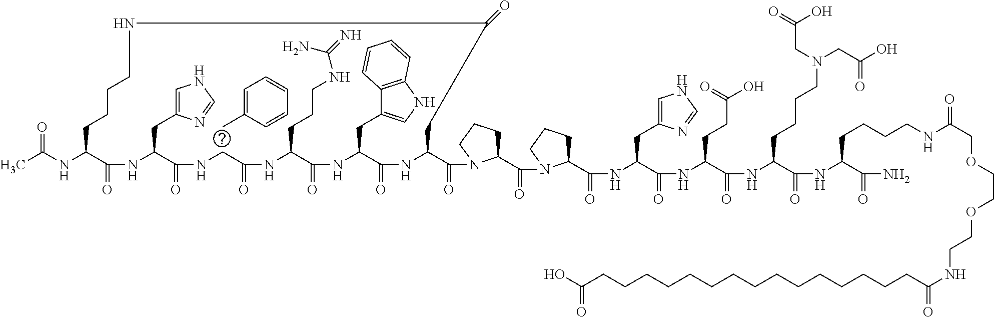 | 17 | >10000 |
| 45 | Ac-c[Lys- His-D-Phe- Arg-Trp-Asp]-Pro- Pro-His-Ser-Lys (biscarboxymethyl)- Ser-Ser-Glu- Lys({2-[2-(16- carboxyhexa- decanoylamino) ethoxy]ethoxy} acetyl)-NH2 | 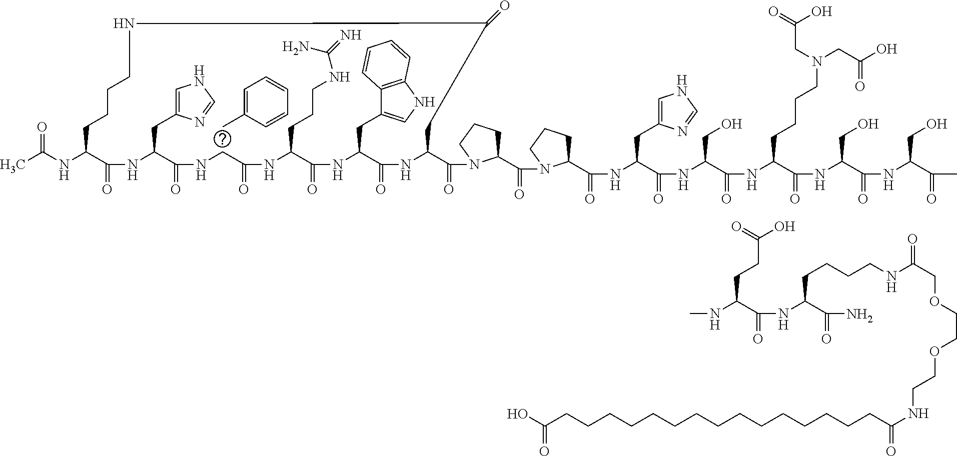 | 14 | >10000 |
| 46 | Ac-c[Orn- His-D-Phe- Arg-Trp-Asp]-Pro- Pro-His-Glu-Lys (biscarboxymethyl)- Lys({2-[2-(17- carboxyhepta- decanoylamino) ethoxy]ethoxy} acetyl)-NH2 | 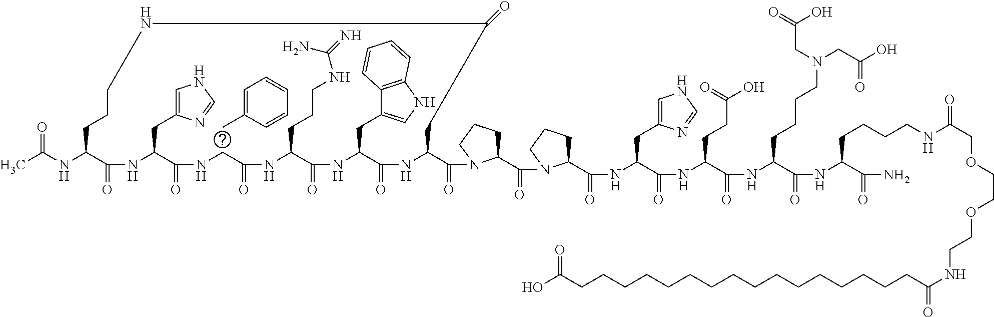 | 1.8 | 7373 |
| 47 | Ac-c[Dap- His-D-Phe- Arg-Trp-Glu]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 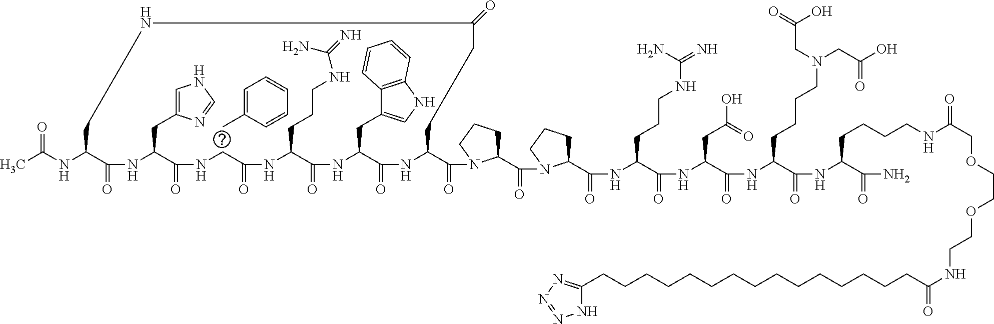 | 0.7 | 3854 |
| 48 | Ac-c[Glu- His-D-Phe- Arg-Trp-Lys]-Hyp- Hyp-Arg-Asp-Lys (biscarboxymethyl)- Lys[(2-{2- [16-(tetrazol-5-yl) hexadecanoylamino] ethoxy}ethoxy) acetyl]-NH2 | 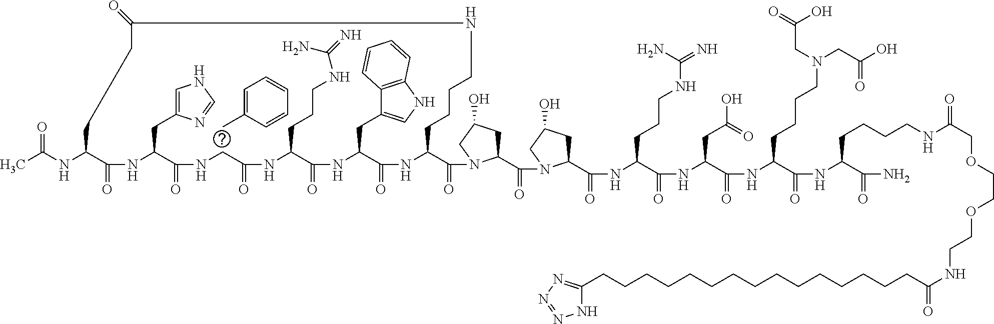 | 21 | >10000 |
| 49 | Ac-c[Lys- His-D-Phe- Arg-Trp-Asp]-Pro- Ser-His-Glu-Lys (biscarboxymethyl)- Lys({2-[2-(16- carboxyhexa- decanoylamino) ethoxy]ethoxy} acetyl)-NH2 | 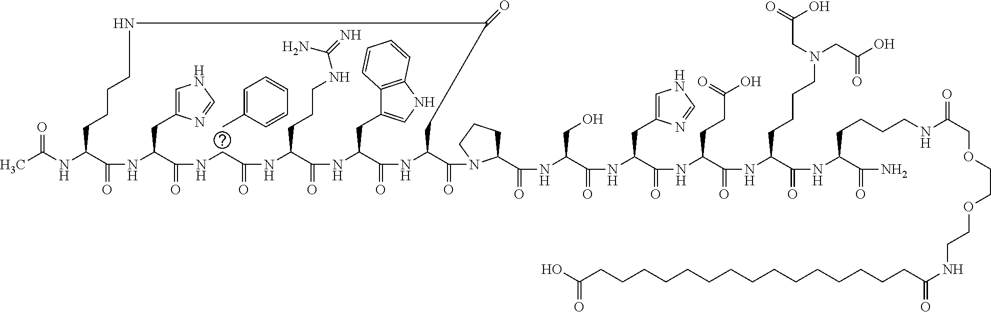 | 25 | >10000 |
| 50 | Ac-c[Orn- His-D-Phe- Arg-Trp-Asp]-Pro- Pro-Arg-Glu-Lys (biscarboxymethyl)- Ser-Ser-NH (2-{2-[2-(17- carboxyhepta- decanoylamino) ethoxy]ethoxy} ethyl) | 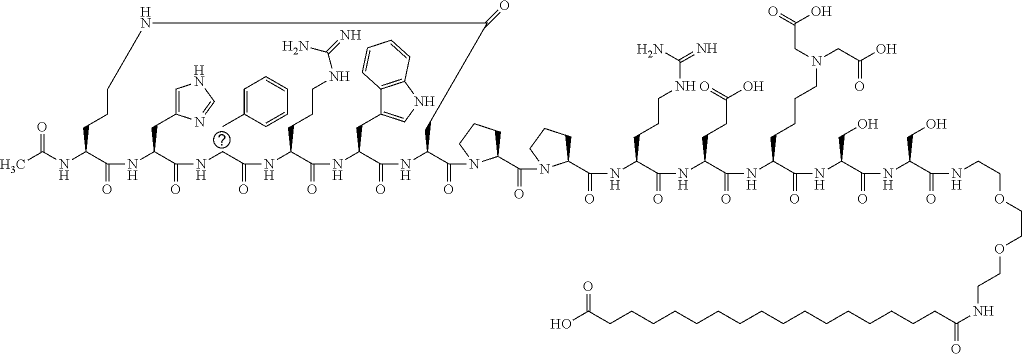 | 0.93 | 2495 |
| 51 | Ac-c[Orn- His-D-Phe- Arg-Trp-Asp]-Pro- Pro-His-Glu-Lys (biscarboxymethyl)- Lys({2-[2-(15- carboxypenta- decanoylamino) ethoxy]ethoxy} acetyl)-NH2 | 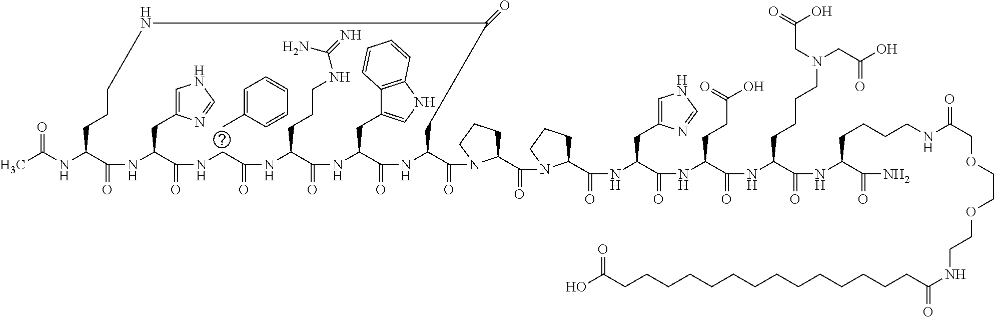 | 3.4 | 8601 |
| 52 | Ac-c[Lys- His-D-Phe- Arg-Trp-Asp]-Pro- Pro-His-Glu- Glu-Ser- Ser-Glu-Lys({2-[2- (16-carboxyhexa- decanoylamino) ethoxy]ethoxy} acetyl)-NH2 | 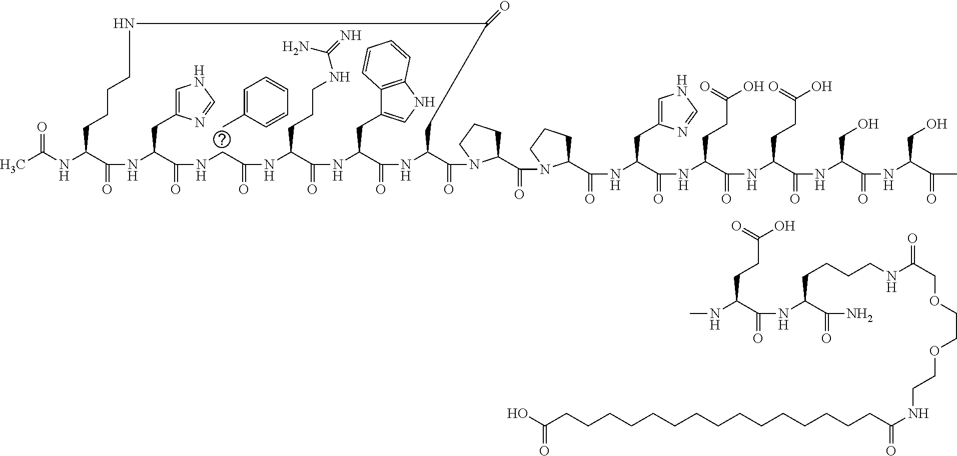 | 30 | >10000 |
| 53 | Ac-c[Lys- His-D-Phe- Arg-Trp-Asp]-Pro- Ser-Ser-Ser-Lys (biscarboxymethyl)- Ser-Ser-NH (2-{2-[2-(17- carboxyhepta- decanoylamino) ethoxy] ethoxy}ethyl) | 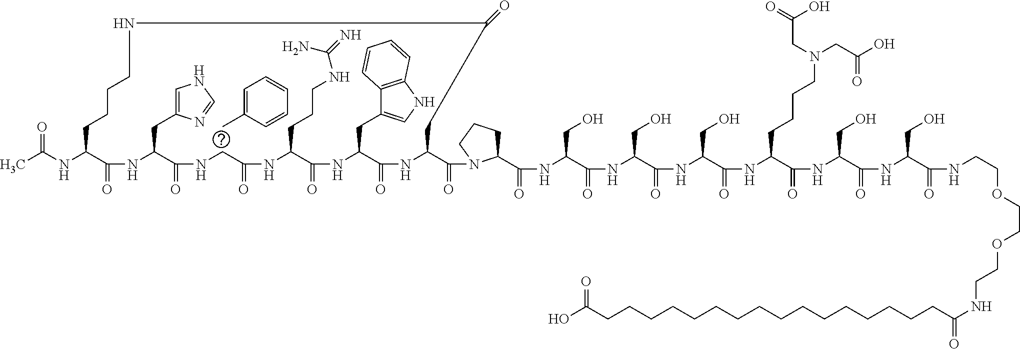 | 2.75 | >10000 |
| 54 | Ac-c[Glu- His-D-Phe- Arg-Trp-Orn]-Pro- Pro-Arg-Asp-Lys (biscarboxymethyl)- Lys[(2-{2- [16-(tetrazol-5-yl) hexadecanoylamino] ethoxy}ethoxy) acetyl]-NH2 | 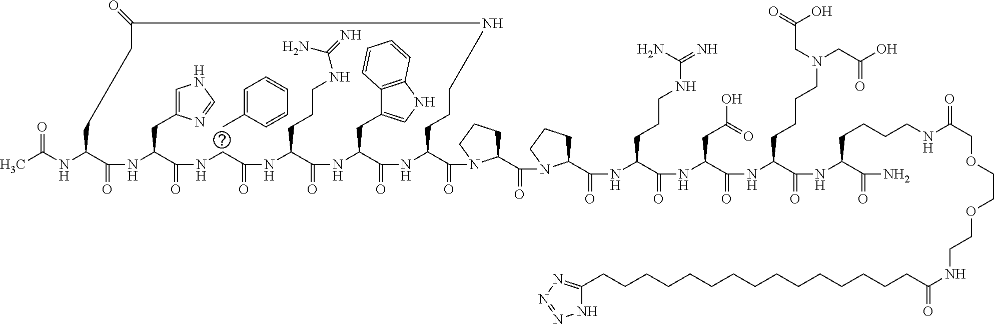 | 1.42 | 5244 |
| 55 | Ac-c[Orn- His-D-Phe- Arg-Trp-Asp]-Pro- Pro-His-Glu-Lys (biscarboxymethyl)- Lys({2-[2-(16- carboxyhexa- decanoylamino) ethoxy]ethoxy} acetyl)-NH2 | 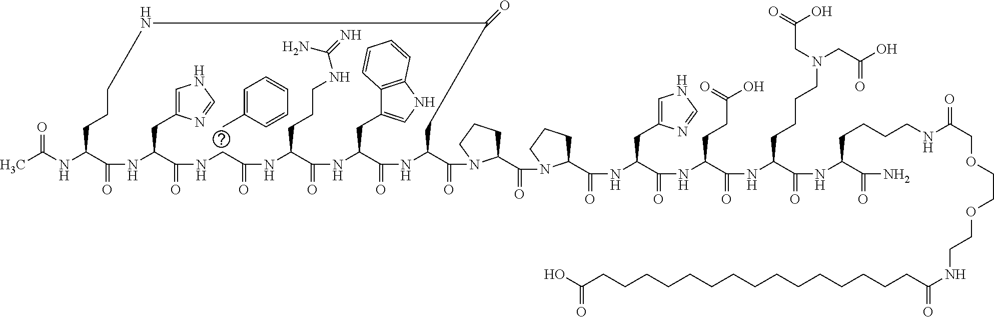 | 1.05 | 4832 |
| 56 | Ac-c[Glu- His-D-Phe- Arg-Trp-Orn]-Hyp- Hyp-Arg-Asp-Lys (biscarboxymethyl)- Lys[(2-{2- [16-(tetrazol-5-yl) hexadecanoylamino] ethoxy}ethoxy) acetyl]-NH2 | 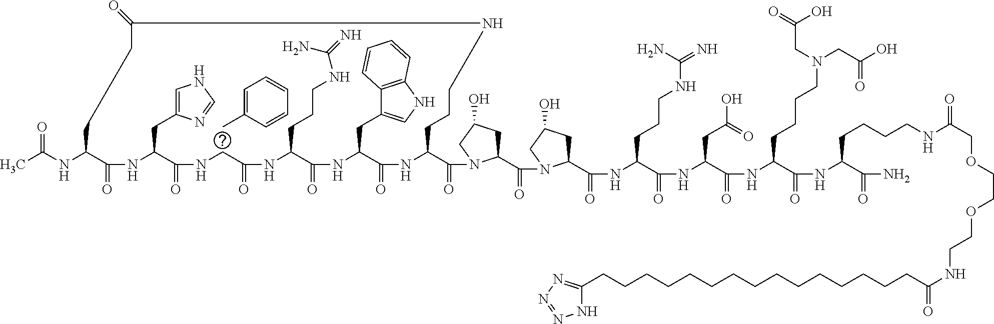 | 3.25 | 7230 |
| 57 | Ac-c[Orn- His-D-Phe- Arg-Trp-Glu]-Pro- Ser-Ser-Ser-Lys (biscarboxymethyl)- Ser-Ser-Lys ({2-[2-(16- carboxyhexa- decanoylamino) ethoxy]ethoxy} acetyl)-NH2 | 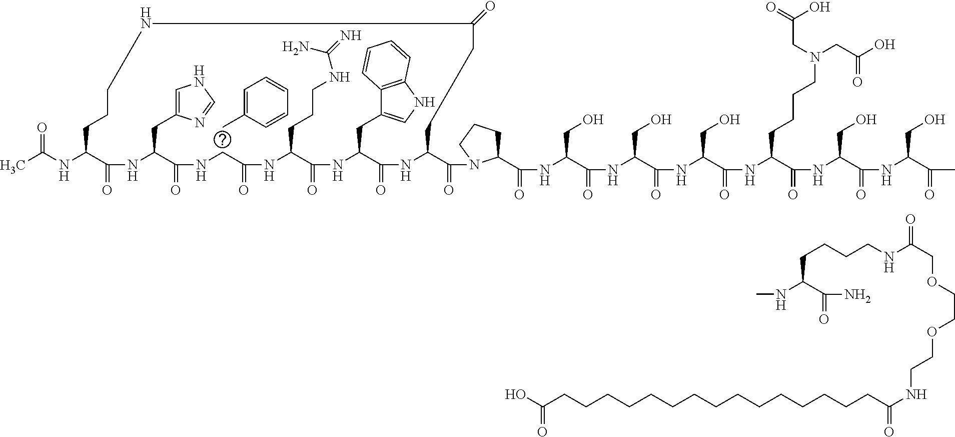 | 6.35 | >10000 |
| 58 | Ac- c[homoCys-Ser- D-Phe-Arg- Trp-Cys]- Pro-Pro-Lys- Glu-Lys (biscarboxymethyl)- Lys[(2-{2- [16-(tetrazol-5-yl) hexadecanoylamino] ethoxy}ethoxy) acetyl]-NH2 | 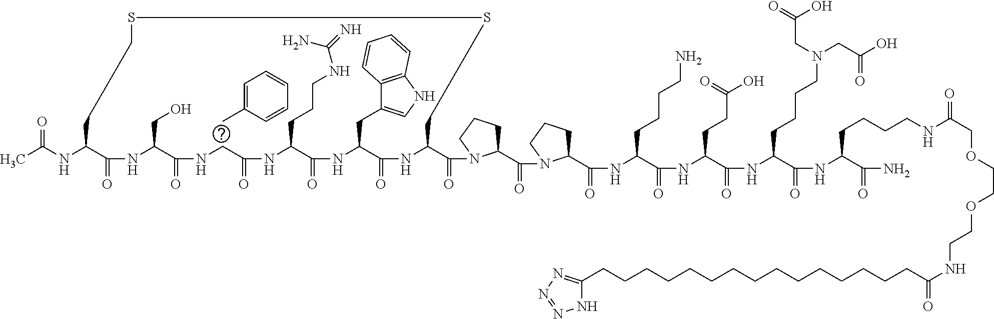 | 10 | 2063 |
| 59 | Ac-c[Cys- Glu-His-D- Phe-Arg-Trp-Gly- Cys]-Pro- Pro-Lys-Glu-Lys (biscarboxymethyl)- Lys({2-[2-(15- carboxypenta- decanoylamino) ethoxy]ethoxy} acetyl)-NH2 | 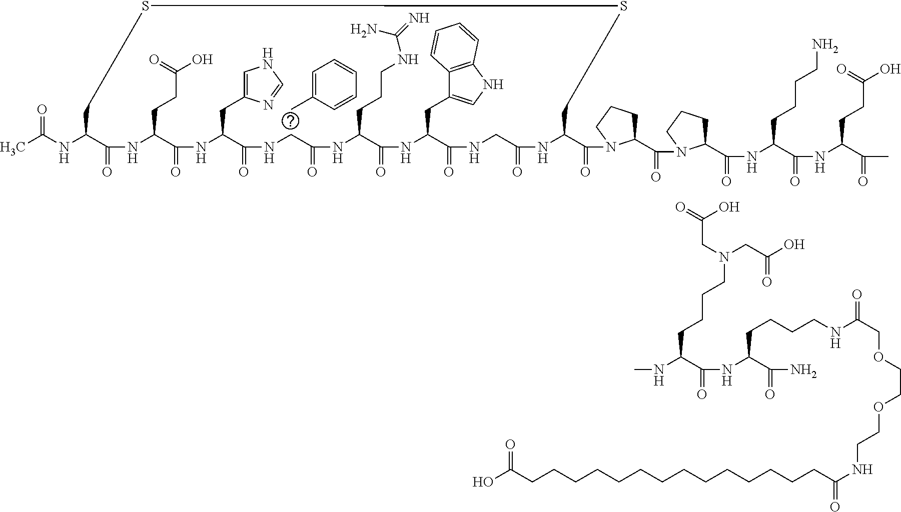 | 55 | >10000 |
| 60 | Ac- c[homoCys-His-D- Phe-Arg-Trp-Cys]- Ser-Pro-Lys- Glu-Lys (biscarboxymethyl)- Lys({2-[2-(15- carboxypenta- decanoylamino) ethoxy]ethoxy} acetyl)-NH2 | 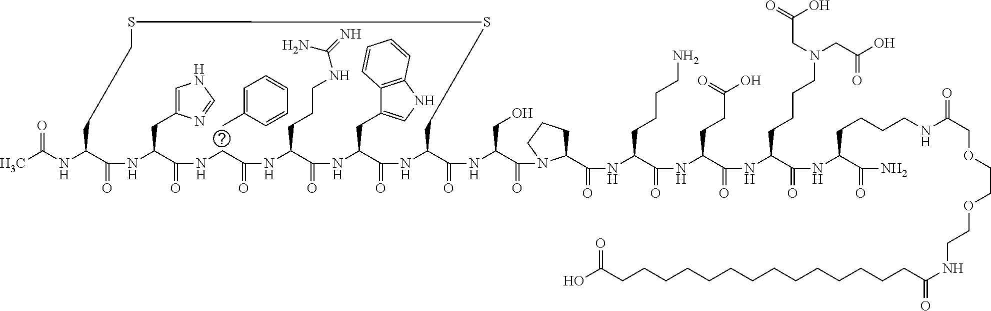 | 3.43 | 169 |
| 61 | Ac- c[homoCys-His-D- Phe-Arg-Trp-Cys]- Pro-Pro-Lys-Asp- Dap{[bis (carboxymethyl) amino]acetyl}- Lys[(2-{2- [16-(tetrazol-5-yl) hexadecanoylamino] ethoxy}ethoxy) acetyl]-NH2 | 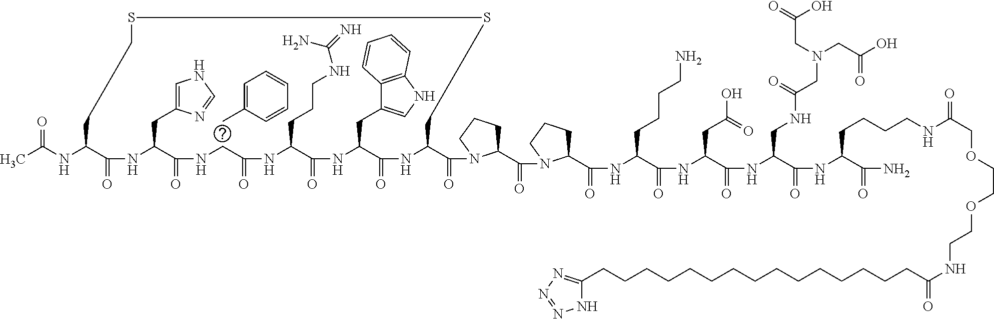 | 0.64 | 7540 |
| 62 | Ac- c[homoCys-His-D- Phe-Arg-Trp-Cys]- Ser-Pro-His- Glu-Lys (biscarboxymethyl)- Lys({2-[2-(17- carboxyhepta- decanoylamino) ethoxy]ethoxy} acetyl)-NH2 | 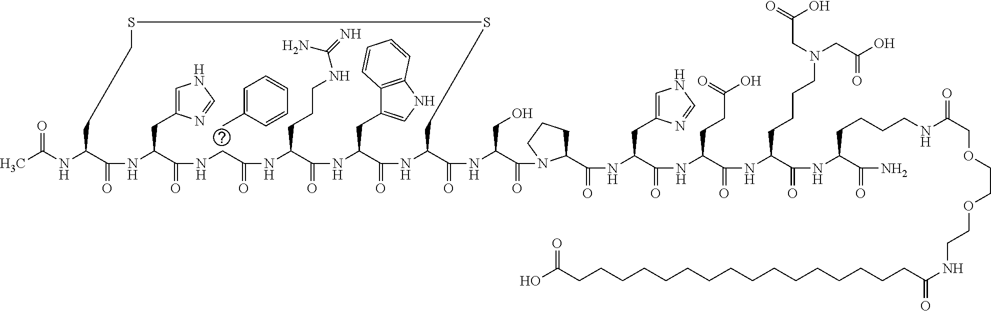 | 0.55 | 442 |
| 63 | Ac-c[Cys- Glu-His-D- Phe-Arg-Trp-Cys]- Pro-Pro-Lys- Glu-Lys (biscarboxymethyl)- Lys[(2-{2- [16-(tetrazol-5-yl) hexadecanoylamino] ethoxy}ethoxy) acetyl]-NH2 | 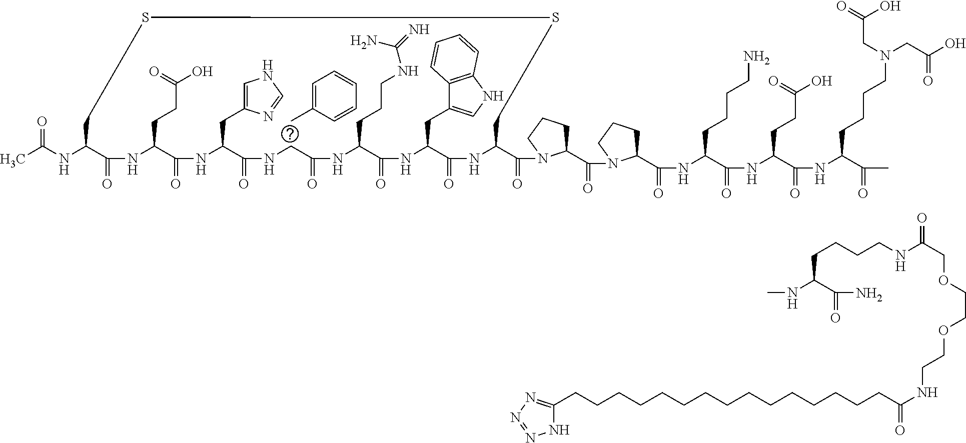 | 15 | >10000 |
| 64 | Ac- c[homoCys-His-D- Phe-Arg-Trp-Cys]- Pro-Pro-Lys-Glu- Dap{[bis (carboxymethyl) amino]acetyl}- Lys[(2-{2- [16-(tetrazol-5-yl) hexadecanoylamino] ethoxy}ethoxy) acetyl]-NH2 | 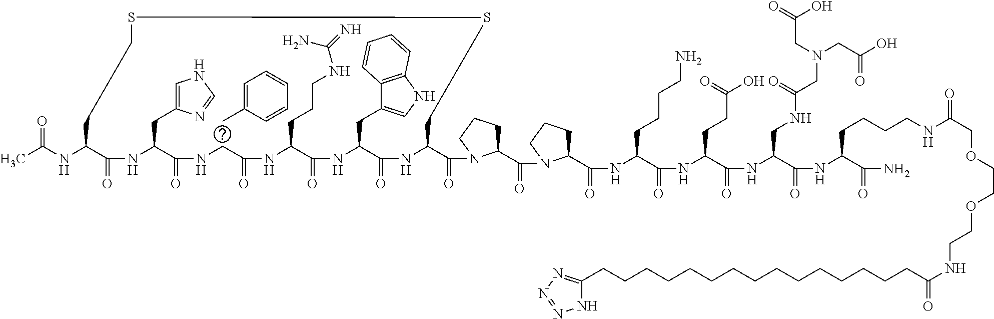 | 0.6 | 177 |
| 65 | Ac- c[homoCys-His-D- Phe-Arg-Trp-Cys]- Pro-Pro-Lys-Asp- Lys{[bis (carboxymethyl) amino]acetyl}- Lys[(2-{2- [16-(tetrazol-5-yl) hexadecanoylamino] ethoxy}ethoxy) acetyl]-NH2 | 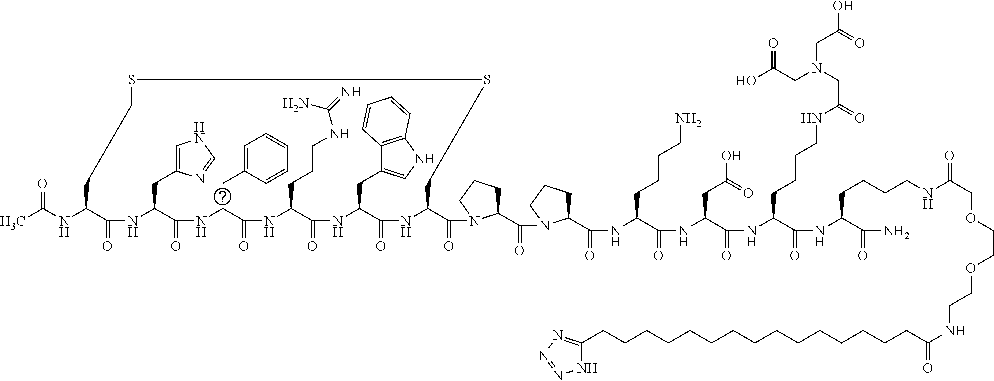 | 0.4 | >10000 |
| 66 | Ac- c[homoCys-Gln-D- Phe-Arg-Trp-Cys]- Pro-Pro-Lys-Asp- Dap{[bis (carboxymethyl) amino]acetyl}- Lys[(2-{2- [16-(tetrazol-5-yl) hexadecanoylamino] ethoxy}ethoxy) acetyl]-NH2 | 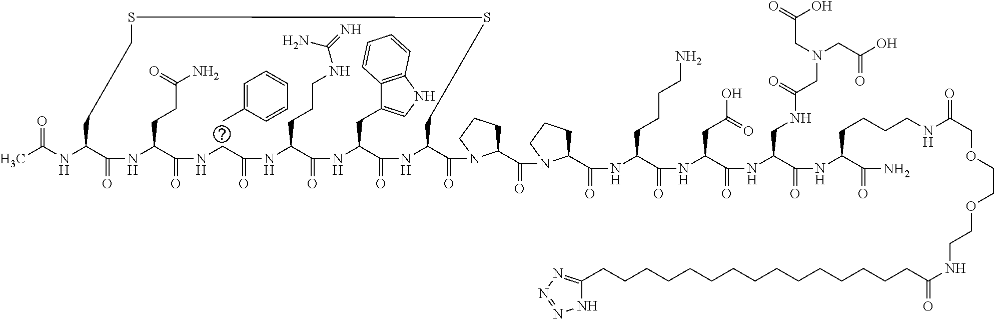 | 1.06 | >10000 |
| 67 | Ac- c[homoCys-Hyp- D-Phe-Arg-Trp- Cys]-Pro-Pro- Lys-Asp- Dap{[bis (carboxymethyl) amino]acetyl}- Lys[(2-{2- [16-(tetrazol-5-yl) hexadecanoylamino] ethoxy}ethoxy) acetyl]-NH2 | 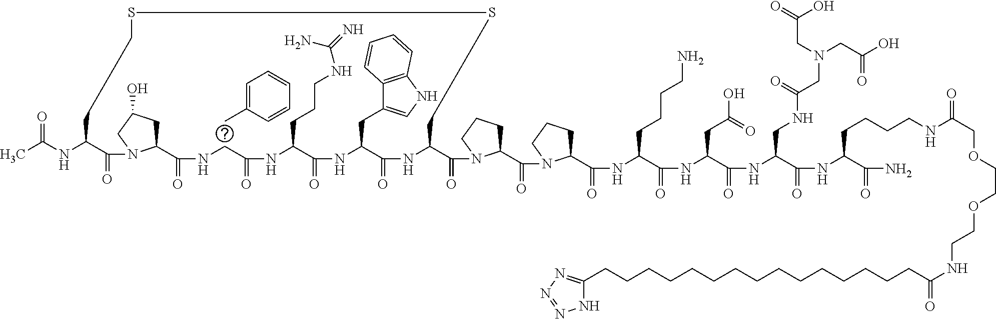 | 0.62 | >10000 |
| 68 | Ac-c[Cys- Glu-His-D- Phe-Arg- Trp-Gly- Cys]-Pro-Pro- Lys-Glu- Glu-Lys ({2-[2-(15- carboxypenta- decanoylamino) ethoxy]ethoxy} acetyl)-NH2 | 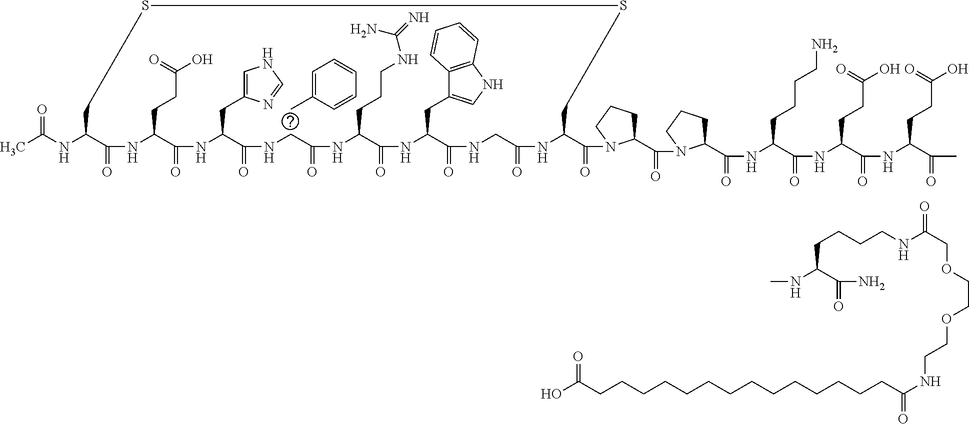 | 6.2 | 7216 |
| 69 | Ac- c[homoCys- His-D- Phe-Arg- Trp-Cys]- Pro-Pro- Lys-Asp- Dap{[bis (carboxymethyl) amino]acetyl}- Lys({2-[2-(13- carboxytride- canoylamino) ethoxy]ethoxy} acetyl)-NH2 | 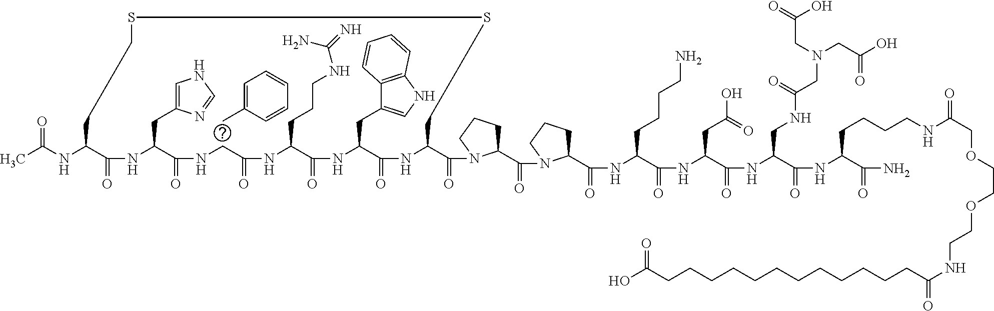 | 4.2 | >10000 |
| 70 | Ac- c[homoCys- His-D- Phe-Arg- Trp-Cys]- Pro-Pro-Ser- Glu-Lys (biscarboxymethyl)- Lys({2-[2-(15- carboxypenta- decanoylamino) ethoxy] ethoxy}acetyl)- NH2 | 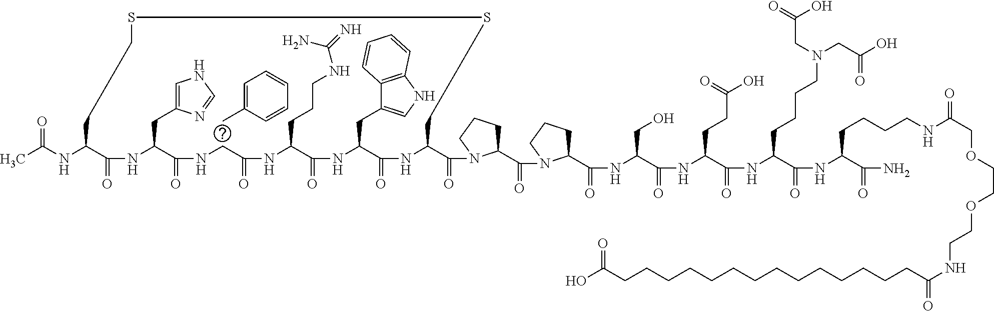 | 70 | >10000 |

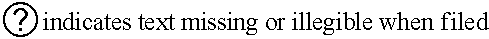

### Assay (V)—Melanocortin Receptor 4 (MC4) Binding Assay

### In Vitro ¹²⁵NDP-α-MSH Binding to Recombinant BHK Cells Expressing Human MC4 Receptor (Filtration Assay).

The assay was performed in 5 ml minisorb vials (Sarstedt No. 55.526) or in 96-well filter-plates (Millipore MADVN 6550), and using BHK cells expressing the human MC4 receptor using BHK cells stably expressing the human MC4 receptor. The membranes were prepared from frozen or fresh cells that were homogenized in 20 mM HEPES pH 7.1, 5 mM MgCl₂ and 1 mg/ml bacitracin and centrifuged at 15000 rpm at 4° C., 10 min in a Sorvall RC 5B plus, SS-34 rotor. The supernatant was discarded, and the pellets were re-suspended in buffer, homogenized and centrifuged two more times. The final pellets were resuspended in the buffer mentioned above, and the protein concentration was measured and adjusted with buffer to 14 to 17 mg/ml and the membrane preparation were kept at −80° C. until assay. The assay was run directly on a dilution of this cell membrane suspension, without any further preparation. The BHK cell membranes are kept at −80° C. until assay, and the assay is run directly on a dilution of this cell membrane suspension, without further preparation. The suspension is diluted to give maximally 10% specific binding, i.e. to approx. 50-100 fold dilution. The assay was performed in a total volume of 200 μl: 50 μl of cell suspension, 50 μl of ¹²⁵NDP-α-MSH (≈79 pM in final concentration), 50 μl of test compound and 50 μl binding buffer (pH 7) mixed and incubated for 2 h at 25° C. [binding buffer: 25 mM HEPES, pH 7.0, 1 mM CaCl₂, 1 mM MgSO₄, 1 mM EGTA, 0.02% Bacitracin, 0.005% Tween™ 20 and 0.1% HSA or, alternatively, 0.1% ovalbumin (Sigma; catalogue No. A-5503)]. Test compounds were dissolved in DMSO and diluted in binding buffer. Radiolabelled ligand and membranes were diluted in binding buffer. The incubation was stopped by dilution with 2×100 μl ice-cold 0.9% NaCl. The radioactivity retained on the filters was counted using a Cobra II auto gamma counter.

The data were analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight)^{0.75}] for oxygen consumption and carbon dioxide production, and per animal for heat. Oxygen consumption (VO₂) is regarded as the major energy expenditure parameter of interest.

### Assay (VII)—Evaluation of Binding to Albumin

Test compounds are tested in a functional assay (Assay III) and a binding assay (Assay V),

wherein Assay III contains HSA, and Assay V contains ovalbumin. EC₅₀ values are determined from Assay III, and Ki values from Assay V. The ratio EC₅₀/Ki is then calculated. In the event of no albumin binding the ratio EC₅₀/Ki will be 1 or below. The stronger the binding to albumin, the higher will be the ratio; for albumin-binding test compounds, the ratio EC₅₀/Ki will thus be ≧1 such as ≧10, e.g. ≧100.

### Assay (VIII)—Melanocortin Receptor 3 (MC3) Binding Assay

The MC3 receptor binding assay is performed on BHK cell membranes stably expressing the human MC3 receptor. The human MC3 receptor is cloned by PCR and subcloned into pcDNA3 expression vector. Cells stably expressing the human MC3 receptor are generated by transfecting the expression vector into BHK cells and using G418 to select for MC3 clones. The BHK MC3 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell-suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test compound, 25 μl ¹²⁵I-NDP-α-MSH (app. 60 000 cpm/well ˜0.25 nM in final concentration) and 50 μl membranes (30 μg/well) and incubate in Costar round-bottom wells microtiter plate, (catalogue number 3365). Test-compounds are dissolved in DMSO or H₂O. Radioligand, membranes and test compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-6-cyclodextrin 97%, (Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% polyethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results; IC₅₀ values are calculated by non-linear regression analysis of binding curves (6 points minimum) using the windows program Graph Pad Prism, Graph Pad software, USA. Ki-values were calculated according to the Cheng-Prusoff equation [Y-C. Cheng and W. H. Prusoff, Biochem. Pharmacol. 22 (1973) pp. 3099-3108].

### Assay (IX)—Melanocortin Receptor 5 (MC5) Binding Assay

The MC5 receptor binding assay is performed on BHK cell membranes stably expressing the human MC3 receptor. The human MC5 receptor is cloned by PCR and subcloned into pcDNA3 expression vector. Cells stably expressing the human MC5 receptor are generated by transfecting the expression vector into BHK cells and using G418 to select for MC5 clones. The BHK MC5 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell-suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test-compound, 25-NDP-α-MSH (app. 60 000 cpm/well ˜0.25 nM in final concentration) and 50 μl membranes (10 μg/well) and incubate incubation in Costar round-bottom wells microtiter plate, catalogue number 3365: Test-compounds are dissolved in DMSO or H₂O. Radioligand, membranes and test-compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-β-cyclodextrin, (97%, Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% polyethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results: IC₅₀ values are calculated by non-linear regression analysis of binding curves (6 points minimum) using the windows program Graph Pad Prism, Graph Pad software, USA. Ki-values were calculated according to the Cheng-Prusoff equation [Y-C. Cheng and W. H. Prusoff, Biochem. Pharmacol. 22 (1973) pp. 3099-3108].

Assay (X)—Melanocortin Receptor 3 (MC3) or Melanocortin Receptor 5 (MC5) cAMP Functional Assay Using the FlashPlate® cAMP Detection Kit

The MC3 or MC5-containing BHK cells are stimulated with potential MC3 or MC5 agonists, and the degree of stimulation of cAMP is measured using the Flash Plate® cAMP assay, cat. No SMP004, NEN™ Life Science Products.

### BHK/hMC3 or BHK/hMCS Cells

The cells are produced by transfecting the cDNA encoding MC3 or MC5 receptor into BHK570, and selecting for stable clones expressing the hMC3 receptor. The cells are grown in DMEM, 10% FCS, 1 mg/ml G418 and 1% pen/strep.

Cells at approx. 80-90% confluence are washed with PBS, lifted from the plates with Versene and diluted in PBS. After centrifugation for 5 min at 1300 rpm the supernatant is removed, and the cells are resuspended in stimulation buffer to a final concentration of 2×10⁶ cells/ml. 50 μl cell suspension is added to the Flashplate containing 50 μl of test-compound or reference compound (all dissolved in DMSO and diluted in 0.1% HSA (Sigma A-1887) and 0.005% Tween 20). The mixture is shaken for 5 minutes and then allowed to stand for 25 minutes at room temperature. The reaction is stopped with 100 μl Detection Mix pro well (Detection Mix 11 ml Detection Buffer+100 μl (˜2 μCi) cAMP [¹²⁵I] Tracer). The plates are then sealed with plastic, shaken for 30 minutes and allowed to stand overnight (or for 2 h), and then counted in the Topcounter, 2 min/well (Note that in general, the assay procedure described in the kit-protocol is followed; however, the cAMP standards are diluted in 0.1% HSA and 0.005% Tween 20, and not in stimulation buffer).

### Results

EC₅₀ values are calculated by non-linear regression analysis of dose-response curves (6 points minimum) using the Windows program GraphPad Prism, GraphPad software, USA. Results are expressed in nM. Eₘₐₓ values are calculated as % of NDP-α-MSH maximal stimulation in the hMC3cAMP assay (maximal NDP-α-MSH stimulation 100%).

Assay (XI)—GLP-1 Activity Assay—Stimulation of cAMP Formation in a Cell Line Expressing the Cloned Human GLP-1 Receptor

The following assay may be used to determine the activity (potency) of GLP-1 compounds. In brief, the ability of GLP-1 compounds to stimulate formation of cyclic AMP (cAMP) in a medium containing the human GLP-1 receptor is measured.

In principle, purified plasma membranes from a stable transfected cell line, BHK467-12A (tk-ts13), expressing the human GLP-1 receptor are stimulated with the GLP-1 compound in question, and the potency of cAMP production is measured using the AlphaScreen™ cAMP Assay Kit from Perkin Elmer Life Sciences.

The cells are grown at 5% CO₂ in DMEM, 5% FCS, 1% Pen/Strep (Penicillin/Streptomycin) and 0.5 mg/ml of the selection marker G418.

Cells at approximate 80% confluence are washed 2× with PBS (Phosphate Buffered Saline) and harvested with Versene (aqueous solution of the tetrasodium salt of ethylenediamine-tetraacetic acid), centrifuged 5 min at 1000 rpm and the supernatant removed. The additional steps are all made on ice. The cell pellet is homogenized by the Ultrathurax mixed for 20-30 sec. in 10 ml of Buffer 1 (20 mM Na-HEPES, 10 mM EDTA, pH=7.4), centrifuged 15 min at 20.000 rpm and resuspended in 10 ml of Buffer 2 (20 mM Na-HEPES, 0.1 mM EDTA, pH=7.4). The suspension is homogenized for 20-30 sec and centrifuged 15 min at 20.000 rpm. Suspension in Buffer 2, homogenization and centrifugation is repeated once and the membranes are resuspended in Buffer 2 and ready for further analysis or stored at −80° C. The functional receptor assay is carried out by measuring the peptide induced cAMP production by The AlphaScreen Technology. The basic principle of The AlphaScreen Technology is a competition between endogenous cAMP and exogenously added biotin-cAMP. The capture of cAMP is achieved by using a specific antibody conjugated to acceptor beads. Formed cAMP is counted and measured at an AlphaFusion Microplate Analyzer. The EC₅₀ values are calculated, e.g. using the Graph-Pad Prism software (version 5).

The EC₅₀ values may be indicated relative to, e.g., the EC₅₀ for compound G1. The EC₅₀ values of compounds G2 and G3 relative to that of compound G1 were about 5 times, and 3 times higher, respectively, while the EC50 value of the compound of SEQ ID NO: 4 was about 0.3 times that of compound G1.

1. A compound according to formula I or formula II: 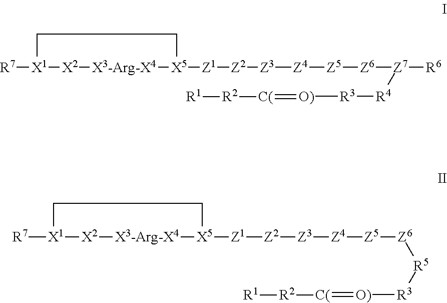
wherein
R¹ represents tetrazol-5-yl or carboxy;
R² represents a straight-chain, branched and/or cyclic C₆₋₂₀alkylene, C₆₋₂₀alkenylene or C₆₋₂₀alkynylene which may optionally be substituted with one or more substituents selected from halogen, hydroxy and aryl;
R³ is absent or represents —NH—S(═O)₂—(CH₂)₃₋₅—C(═O)— or a peptide fragment comprising one or two amino acid residues derived from natural or unnatural amino acids and containing at least one carboxy group;
wherein the side chains of R³ do not contain amino, guanidino, imidazolyl or other basic groups positively charged at neutral pH;
R⁴ in formula I represents a glycolether-based aminoacid structure according to one of the formulas IIIa-IIIh;
—HN—CH₂—CH₂—O—CH₂—CH₂—O—CH₂—C(═O)— [IIIa]
—[HN—CH₂—CH₂—O—CH₂—CH₂—O—CH₂—C(═O)]₂— [IIIb]
—[HN—CH₂—CH₂—O—CH₂—CH₂—O—CH₂—C(═O)]₃₋₅— [IIIc]
—[HN—CH₂—CH₂—O—CH₂—CH₂—O—CH₂—CH₂—NH—C(═O)—CH₂—CH₂—CH₂—C(═O)]₁₋₃— [IIId]
[HN—CH₂—CH₂—O—CH₂—CH₂—O—CH₂—CH₂—NH—C(═O)—CH₂—O—CH₂—C(═O)]₁₋₃— [IIIe]
—[HN—CH₂—CH₂—O—CH₂—CH₂—O—CH₂—CH₂—O—CH₂—CH₂—O—CH₂—CH₂—C(═O)]₁₋₃— [IIIf]
—HN—CH₂—CH₂—[O—CH₂—CH₂]₂₋₁₂—O—CH₂—C(═O)— [IIIg]
—HN—CH₂—CH₂—[O—CH₂—CH₂]₄₋₁₂—O—CH₂—CH₂—C(═O)— [IIIh]
wherein the C-terminus of R⁴ is linked to the side chain of Z⁷;
R⁵ in formula II represents a glycolether-based diamine structure according to one of the formulas IVa-IVe;
—HN—CH₂—CH₂—O—CH₂—CH₂—O—CH₂—CH₂—NH— [IVa]
—HN—CH₂—CH₂—CH₂—O—CH₂—CH₂—O—CH₂—CH₂—CH₂—NH— [IVb]
—HN—CH₂—CH₂—CH₂—[O—CH₂—CH₂]₂—O—CH₂—CH₂—CH₂—NH—[IVc]
—HN—CH₂—CH₂—[O—CH₂—CH₂]₂₋₁₀—O—CH₂—CH₂—NH— [IVd]
—HN—CH₂—CH₂—CH₂—[O—CH₂—CH₂]₃₋₁₀—O—CH₂—CH₂—CH₂—NH— [IVe]
wherein R⁵ is linked to the C-terminus of Z⁶, if Z⁶ is present, or to the C-terminus of Z⁵, if Z⁶ is absent;
R⁶ in formula I represents OR′ or N(R′)₂, wherein each R′ independently represents hydrogen or represents C₁₋₆alkyl, C₂₋₆alkenyl or C₂₋₆alkynyl which may optionally be substituted with one or more hydroxy;
R⁷ represents C₁₋₆alkanoyl, C₂₋₆alkenoyl or C₂₋₆alkynoyl which may optionally be substituted with one or more hydroxy;
X¹ represents Glu, Asp, Cys, homoCys, Lys, Orn, Dab or Dap;
X² represents His, Ser, Thr, Gln, Asn, Cit, Hyp, Pro, Ala, or dipeptide fragment Glu-His or Asp-His;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine, (S)-2,3,4,9-tetrahydro-1H-β-carboline-3-carboxylic acid, or dipeptide fragment Trp-Gly;
X⁵ represents Glu, Asp, Cys, homoCys, Lys, Orn, Dab or Dap;
wherein X¹ and X⁵ are joined, rendering the compound cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;
Z¹ represents Pro, D-Pro, Hyp, D-Hyp, Ser, D-Ser, Thr, D-Thr, Gln, D-Gln, Asn, D-Asn, Cit, D-Cit, Ala or D-Ala;
Z² represents Pro, D-Pro, Hyp, D-Hyp, Ser, D-Ser, Thr, D-Thr, Gln, D-Gln, Asn, D-Asn, Cit, D-Cit, Ala or D-Ala;
wherein at least one of residues Z¹ and Z² is selected from Pro, D-Pro, Hyp or D-Hyp;
Z³ is absent or represents Arg, D-Arg, homoArg, D-homoArg, Lys, D-Lys, homoLys, D-homoLys, Orn, D-Orn, Dab, D-Dab, Dap, D-Dap, Dab(Me₂), D-Dab(Me₂), Dap(Me₂), D-Dap(Me₂), Dab(iPr), D-Dab(iPr), Dap(iPr), D-Dap(iPr), His, D-His, Ser, D-Ser, Thr, D-Thr, Asn, D-Asn, Gln, D-Gln, Cit, D-Cit, Ala, D-Ala, Gly or β-Ala;
Z⁴ represents Glu, D-Glu, Asp, D-Asp, Ser, D-Ser, Thr, D-Thr, Asn, D-Asn, Gln, D-Gln, Cit, D-Cit, Ala, D-Ala, Gly or β-Ala;
with the proviso that Z⁴ is selected from Glu, D-Glu, Asp and D-Asp, if Z³ is Arg, D-Arg, homoArg, D-homoArg, Lys, D-Lys, homoLys, D-homoLys, Orn, D-Orn, Dab, D-Dab, Dap, D-Dap, Dab(Me₂), D-Dab(Me₂), Dap(Me₂), D-Dap(Me₂), Dab(iPr), D-Dab(iPr), Dap(iPr) or D-Dap(iPr);
Z⁵ represents Lys(BCMA), D-Lys(BCMA), homoLys(BCMA), D-homoLys(BCMA), Orn(BCMA), D-Orn(BCMA), Dab(BCMA), D-Dab(BCMA), Dap(BCMA), D-Dap(BCMA), Lys(biscarboxymethyl), D-Lys(biscarboxymethyl), homoLys(biscarboxymethyl), D-homoLys(biscarboxymethyl), Orn(biscarboxymethyl), D-Orn(biscarboxymethyl), Dab(biscarboxymethyl), D-Dab(biscarboxymethyl), Dap(biscarboxymethyl), D-Dap(biscarboxymethyl), Glu, D-Glu, Asp or D-Asp;
Z⁶ is absent or represents a peptide fragment comprising one to four amino acid residues selected from Glu, D-Glu, Asp, D-Asp, Ser, D-Ser, Thr, D-Thr, Asn, D-Asn, Gln, D-Gln, Cit, D-Cit, Ala, D-Ala, Gly or β-Ala;
Z⁷ in formula I represents Lys, D-Lys, homoLys, D-homoLys, Orn, D-Orn, Dab, D-Dab, Dap, or D-Dap;
wherein the amino group in the side chain of Z⁷ is linked to R⁴ via an amide bond;
and pharmaceutically acceptable salts, prodrugs and solvates thereof.

2. A compound according to claim 1, selected from the group consisting of:
Ac-c[Dap-His-D-Phe-Arg-Trp-Asp]-Pro-Pro-Arg-Asp-Lys(BCMA)-Lys[(2-{2-[16-(tetrazol-5-yl)hexadecanoylamino]ethoxy}ethoxy)acetyl]-NH₂ 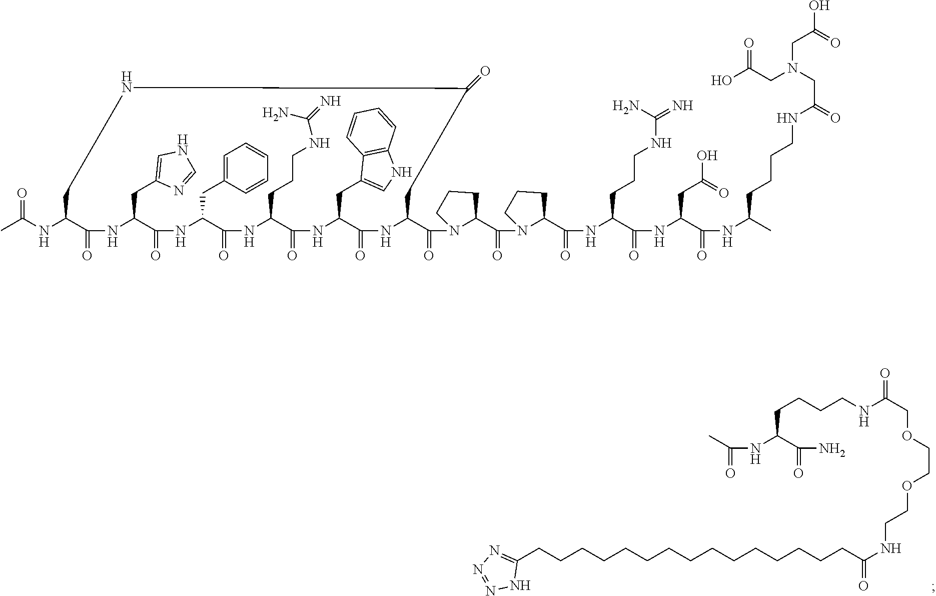 [SMILES: CC(=O)N[C@H]1CNC(=O)C[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@H](C)CCCCNC(=O)CN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(N)=O]
Ac-c[Asp-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 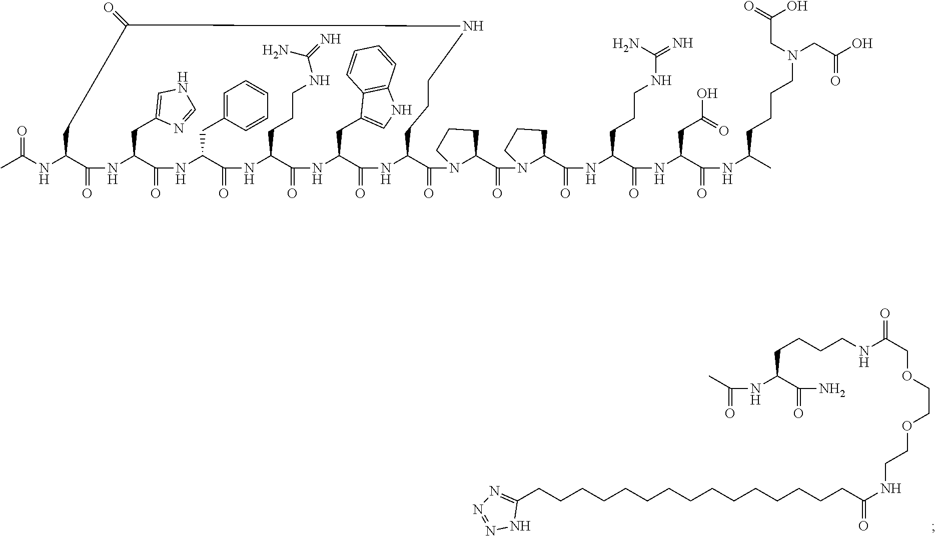 [SMILES: CC(=O)N[C@H]1CC(=O)NCCC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(N)=O]
Ac-c[Dab-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl) 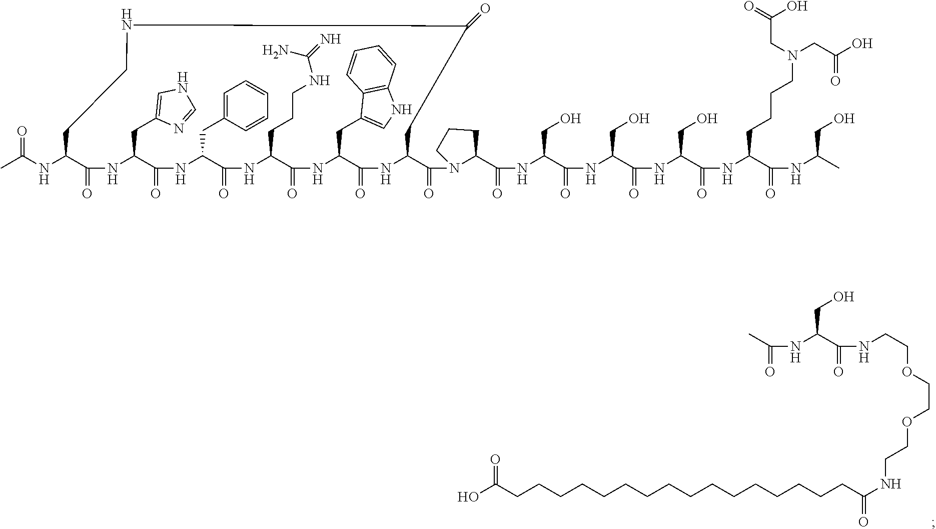 [SMILES: CC(=O)N[C@H]1CCNC(=O)C[C@@H](C(=O)N2CCC[C@H]2C(=O)N[C@@H](CO)C(=O)N[C@@H](CO)C(=O)N[C@@H](CO)C(=O)N[C@@H](CCCCN(CC(=O)O)CC(=O)O)C(=O)N[C@H](C)CO)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CO)C(=O)NCCOCCOCCNC(=O)CCCCCCCCCCCCCCCCC(=O)O]
Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-OH 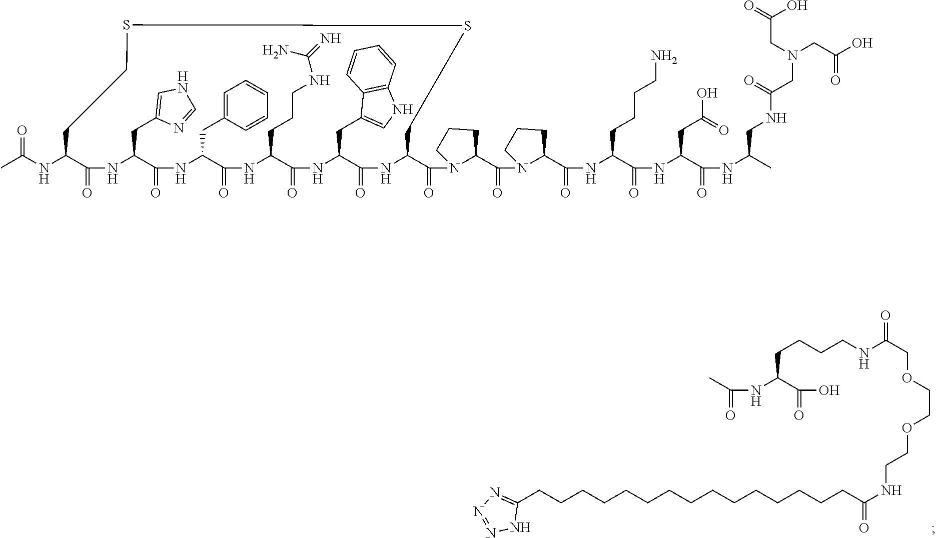 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CC(=O)O)C(=O)N[C@H](C)CNC(=O)CN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(=O)O]
Ac-c[homoCys-His-D-Phe-Arg-Trp-Cys]-Pro-Pro-Lys-Glu-Lys(biscarboxymethyl)-Lys({2-[2(15-carboxypentadecanoylamino)ethoxy]ethoxy}acetyl)-NH₂ 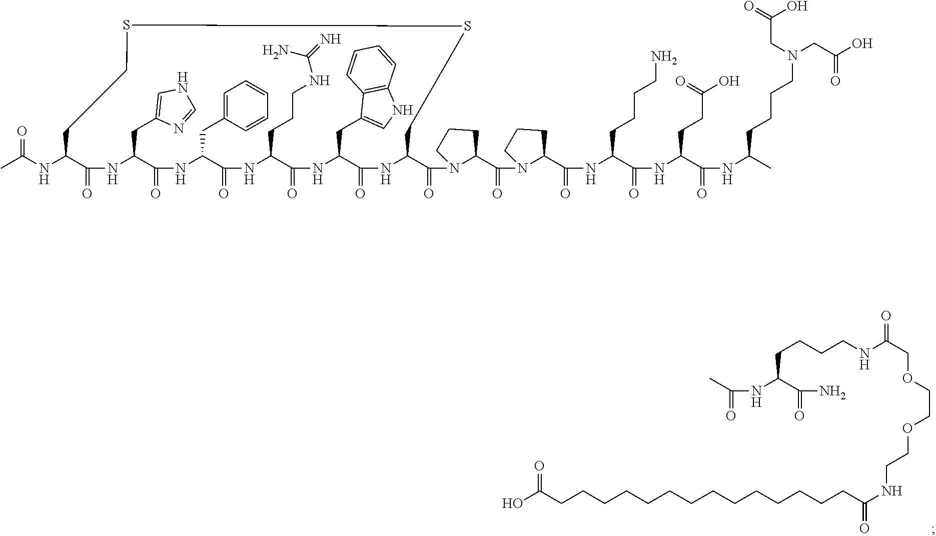 [SMILES: CC(=O)N[C@H]1CCSSC[C@@H](C(=O)N2CCC[C@H]2C(=O)N2CCC[C@H]2C(=O)N[C@@H](CCCCN)C(=O)N[C@@H](CCC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCC(=O)O)C(N)=O]
Ac-c[Glu-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 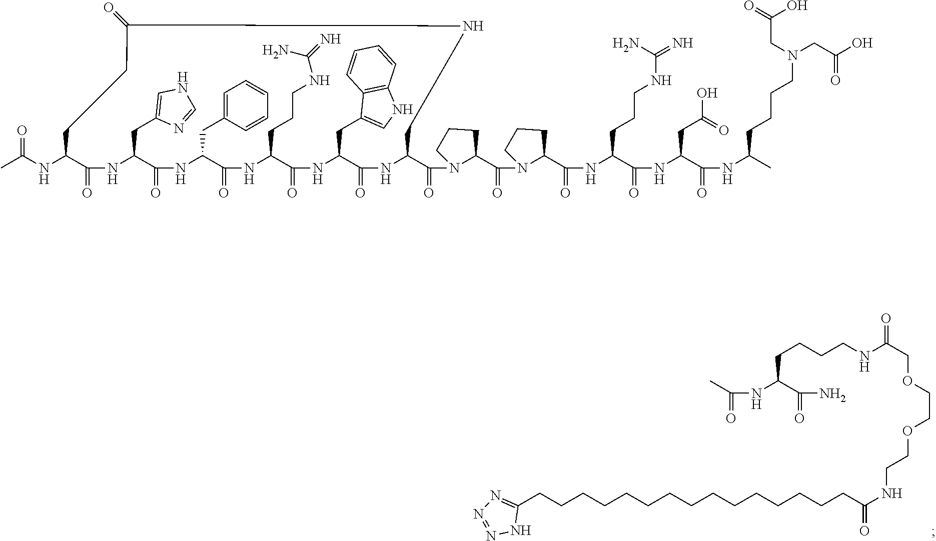 [SMILES: CC(=O)N[C@H]1CCC(=O)NC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(N)=O]
Ac-c[Lys-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂ 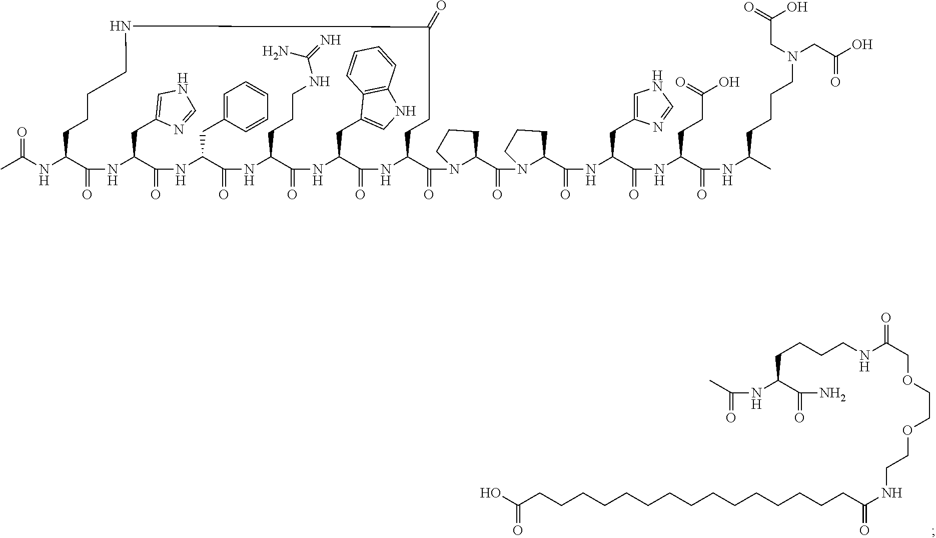 [SMILES: CC(=O)N[C@H]1CCCCNC(=O)CC[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCC(=O)O)C(N)=O]
Ac-c[Orn-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 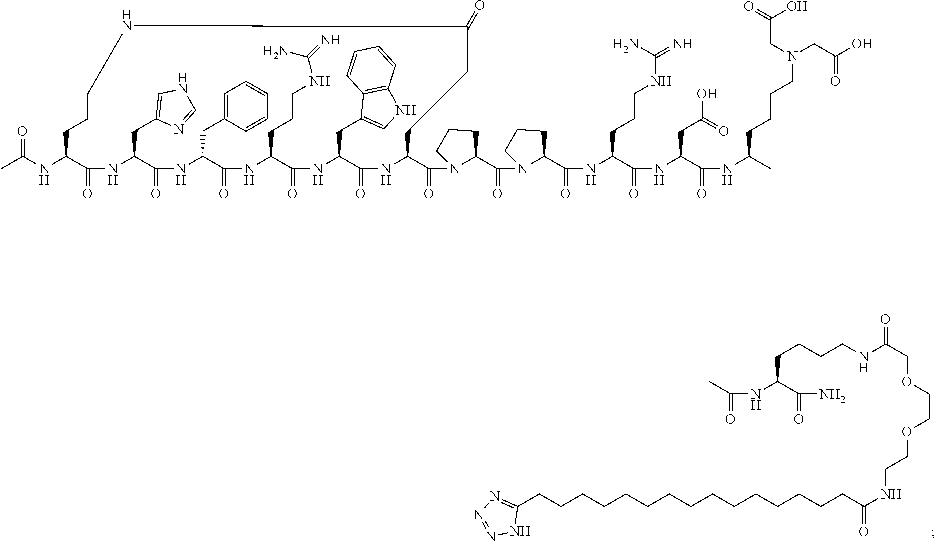 [SMILES: CC(=O)N[C@H]1CCCNC(=O)CC[C@@H](C(=O)N2CCC[C@H]2C(=O)N2CCC[C@H]2C(=O)N[C@@H](CCCNC(=N)N)C(=O)N[C@@H](CC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(N)=O]
Ac-c[Dab-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl) 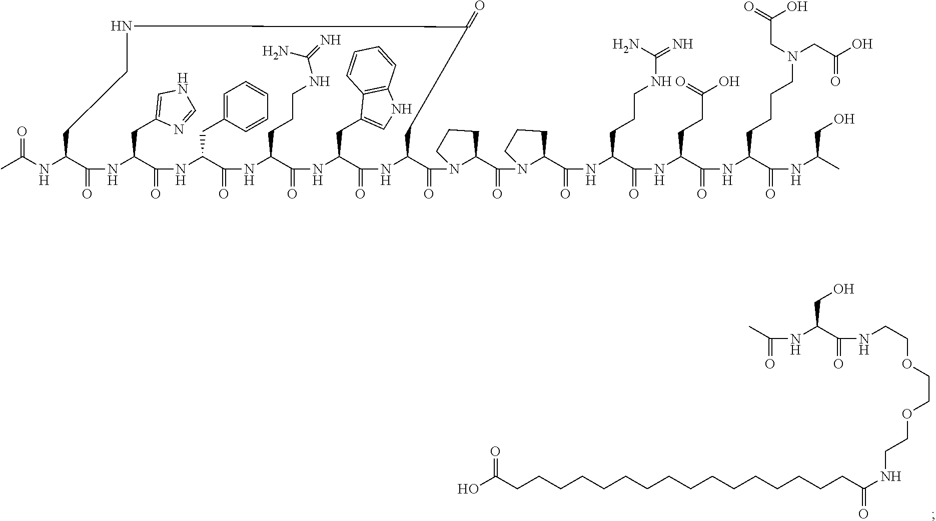 [SMILES: CC(=O)N[C@H]1CCNC(=O)C[C@@H](C(=O)N2CCC[C@H]2C(=O)N2CCC[C@H]2C(=O)N[C@@H](CCCNC(=N)N)C(=O)N[C@@H](CCC(=O)O)C(=O)N[C@@H](CCCCN(CC(=O)O)CC(=O)O)C(=O)N[C@H](C)CO)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CO)C(=O)NCCOCCOCCNC(=O)CCCCCCCCCCCCCCCCC(=O)O]
Ac-c[Orn-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂ 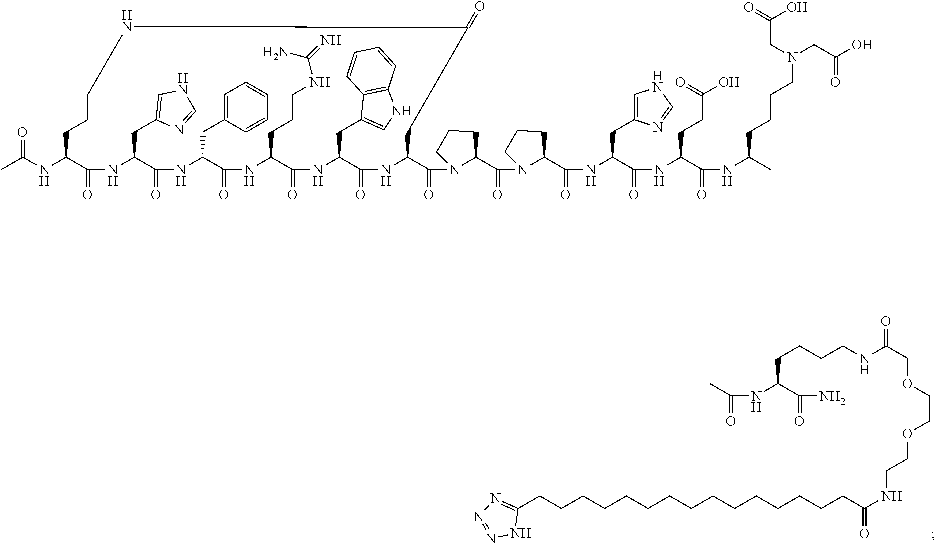 [SMILES: CC(=O)N[C@H]1CCCNC(=O)C[C@@H](C(=O)N2CCC[C@H]2C(=O)N2CCC[C@H]2C(=O)N[C@@H](Cc2c[nH]cn2)C(=O)N[C@@H](CCC(=O)O)C(=O)N[C@H](C)CCCCN(CC(=O)O)CC(=O)O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCCNC(=O)COCCOCCNC(=O)CCCCCCCCCCCCCCCc1nnn[nH]1)C(N)=O]
Ac-c[Lys-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 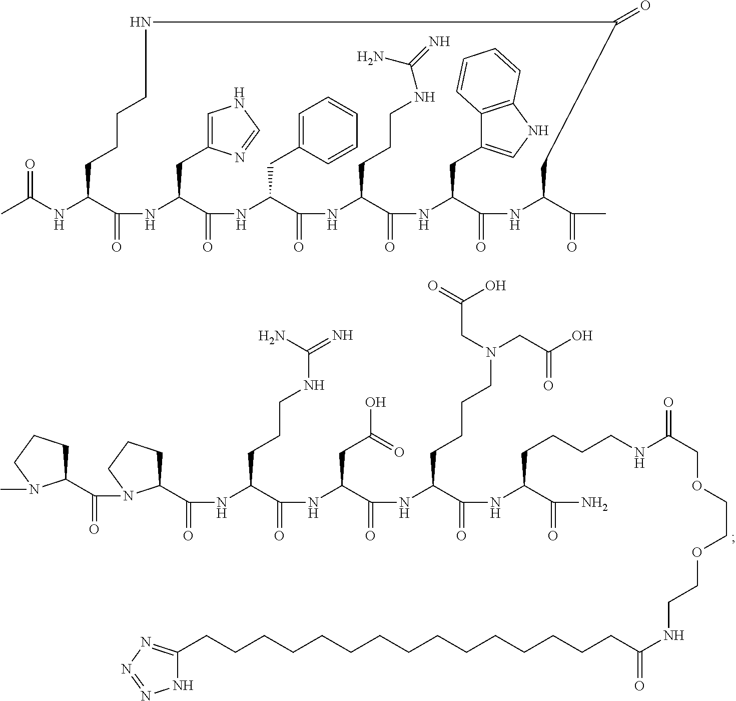
Ac-c[Orn-His-D-Phe-Arg-Trp-Asp]-Pro-Ser-Ser-Ser-Lys(biscarboxymethyl)-Ser-Ser-Lys({2-[2-(15-carboxypentadecanoylamino)ethoxy]ethoxy}acetyl)-NH₂ 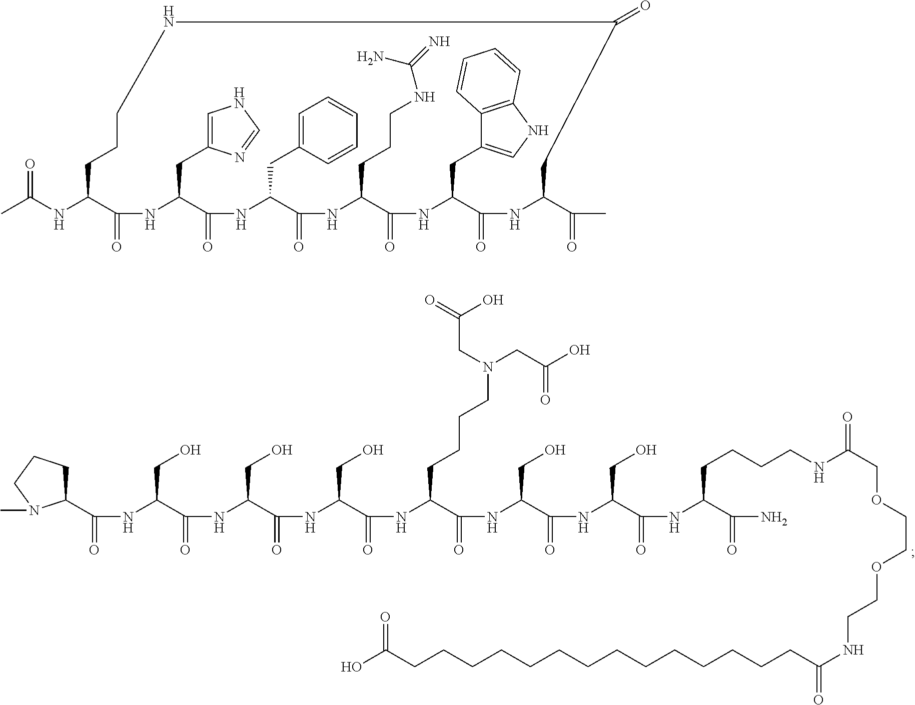 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCC(=O)O)C(N)=O]
Ac-c[Glu-His-D-Phe-Arg-Trp-Orn]-Pro-Pro-Asp-Lys(biscarboxymethyl)-Lys({2-[2-(15-carboxypentadecanoylamino)ethoxy]ethoxy}acetyl)-NH₂ 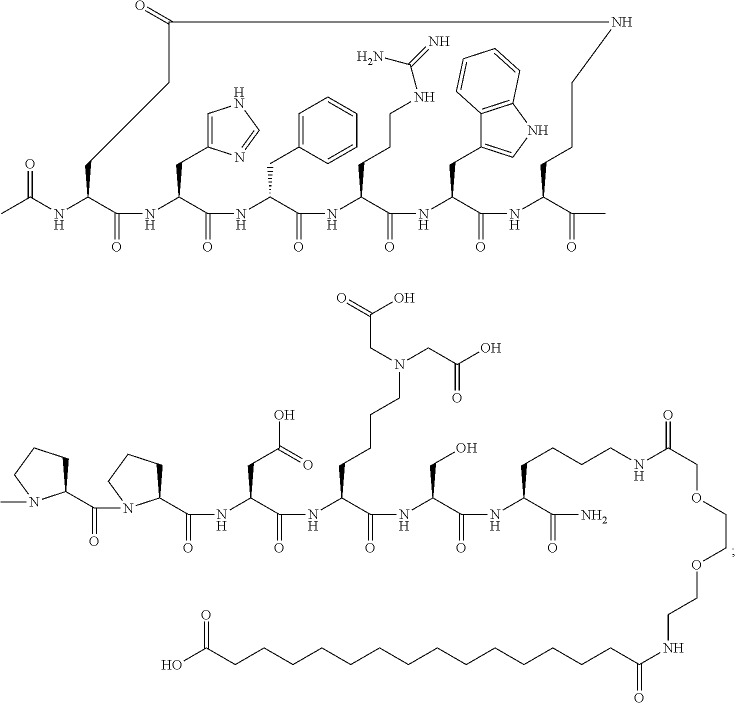 [SMILES: CC(=O)N[C@H]1CCC(=O)NCCC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CC(=O)O)C(=O)N[C@@H](CCCCN(CC(=O)O)CC(=O)O)C(=O)N[C@@H](CO)C(=O)N[C@@H](CCCCNC(=O)COCCOCCNC(=O)CCCCCCCCCCCCCCC(=O)O)C(N)=O]
Ac-c[Orn-His-D-Phe-Arg-Trp-Asp]-Pro-Ser-Ser-Ser-Lys(biscarboxymethyl)-Ser-Ser-Lys({2-[2-(17-carboxyheptadecanoylamino)ethoxy]ethoxy}acetyl)-NH₂ 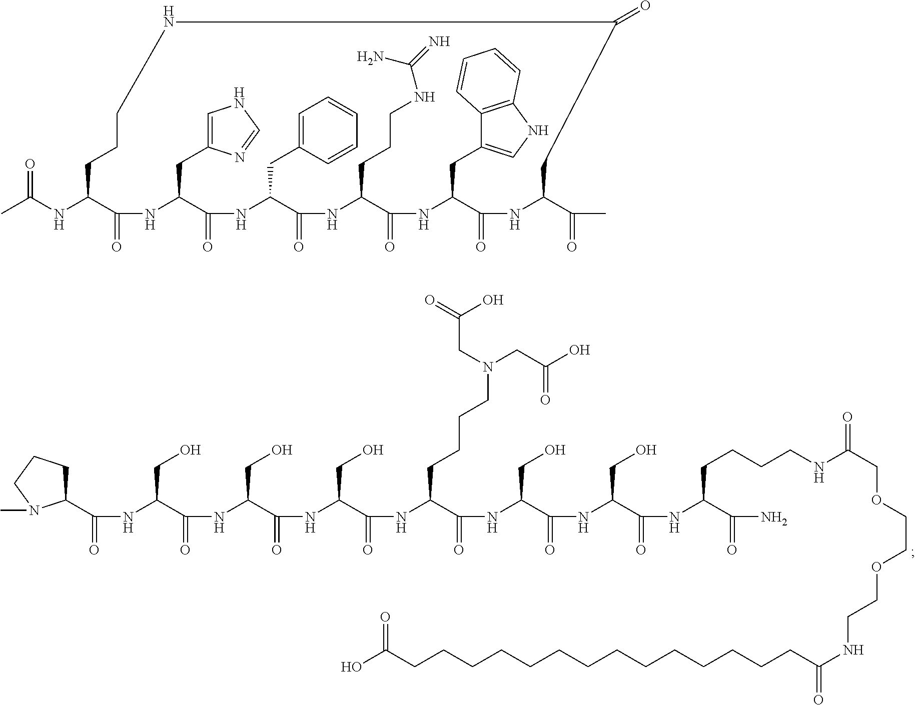 [SMILES: CC(=O)N[C@H]1CCCNC(=O)C[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)N[C@@H](CCCCNC(=O)COCCOCCNC(=O)CCCCCCCCCCCCCCC(=O)O)C(N)=O]
Ac-c[Dab-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂ 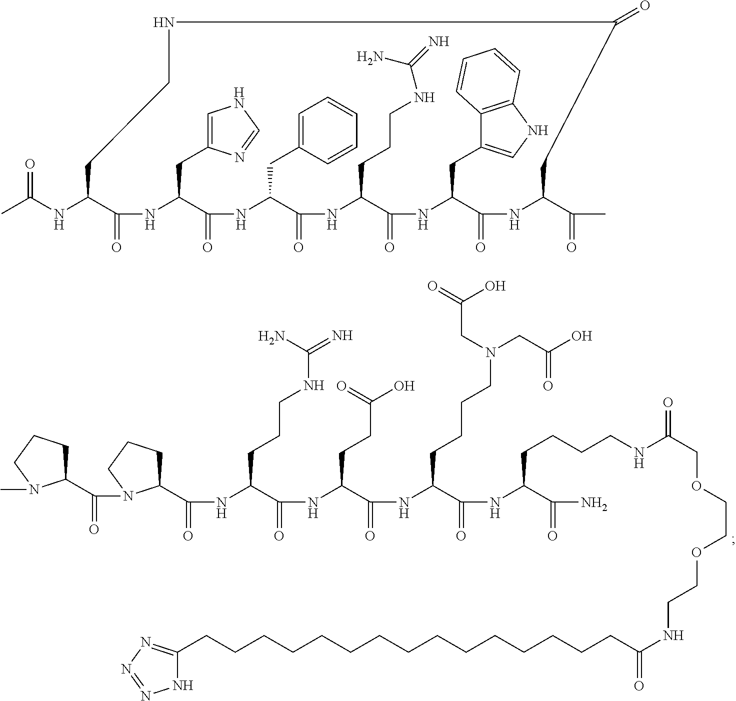
Ac-c[Glu-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 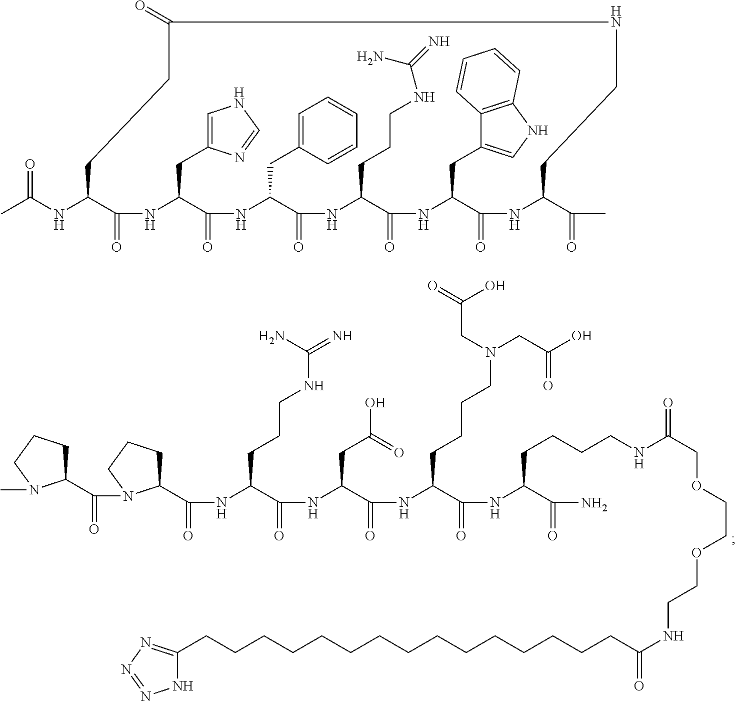
Ac-c[Orn-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 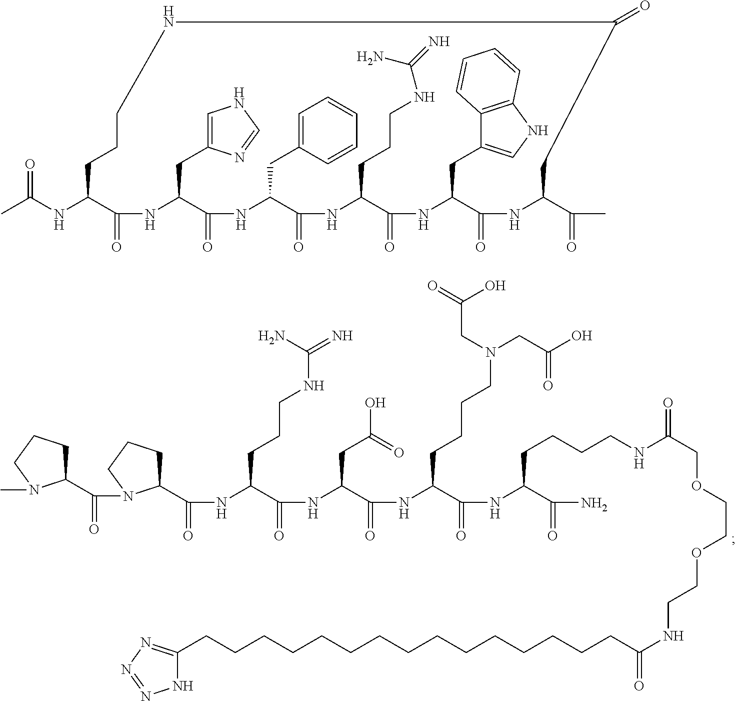
Ac-c[Glu-His-D-Phe-Arg-Trp-Orn]-Pro-Pro-Arg-Asp-Lys(biscarboxymethyl)-Lys({2-[2-(15-carboxypentadecanoylamino)ethoxy]ethoxy}acetyl)-NH₂ 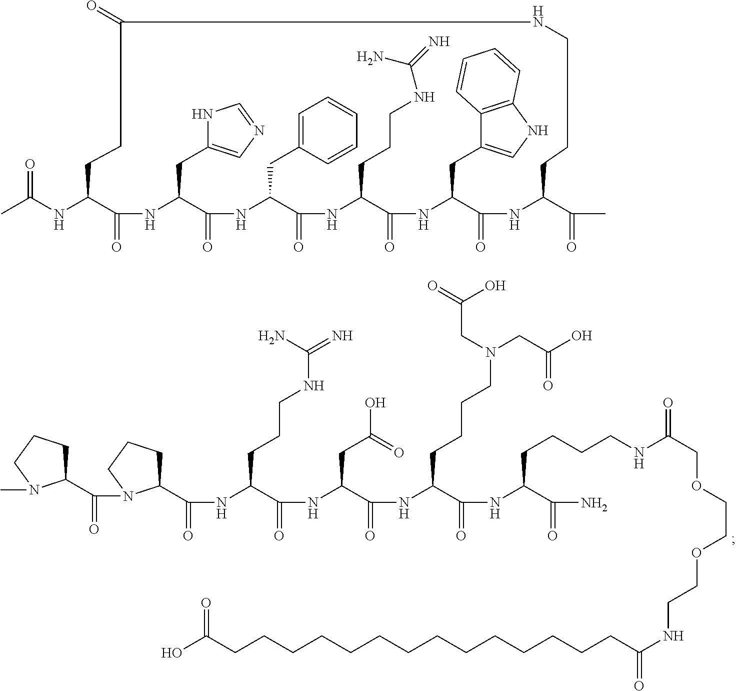
Ac-c[Orn-His-D-Phe-Arg-Trp-Asp]-Pro-Pro-Arg-Glu-Lys(biscarboxymethyl)-Lys[(2-{2-[16-(tetrazol-5-yl)hexadecanoylamino]ethoxy}ethoxy)acetyl]-NH₂ 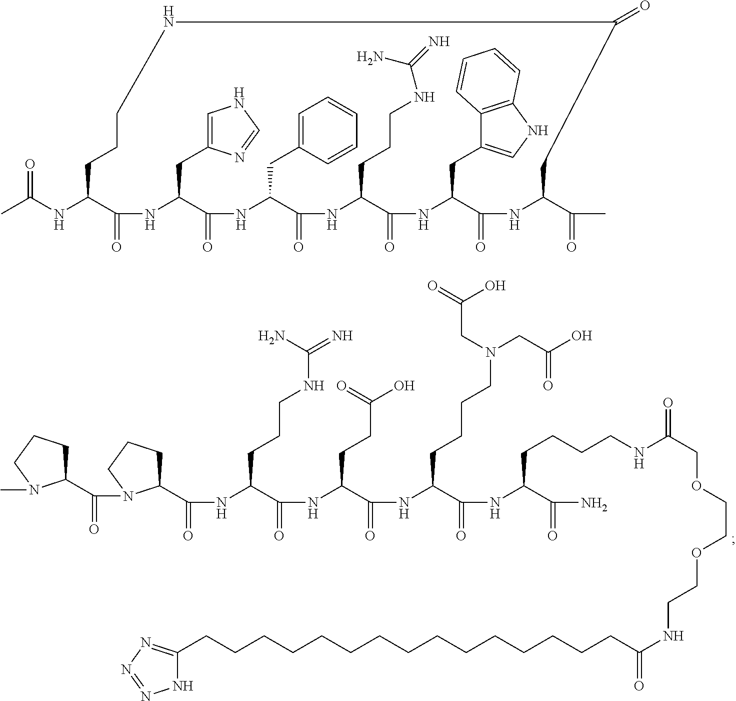
Ac-c[Orn-His-D-Phe-Arg-Trp-Glu]-Pro-Ser-Lys-Glu-Glu-Ser-Ser-Glu-Lys({2-[2-(16-carboxyhexadecanoylamino)ethoxy]ethoxy}acetyl)-NH₂ 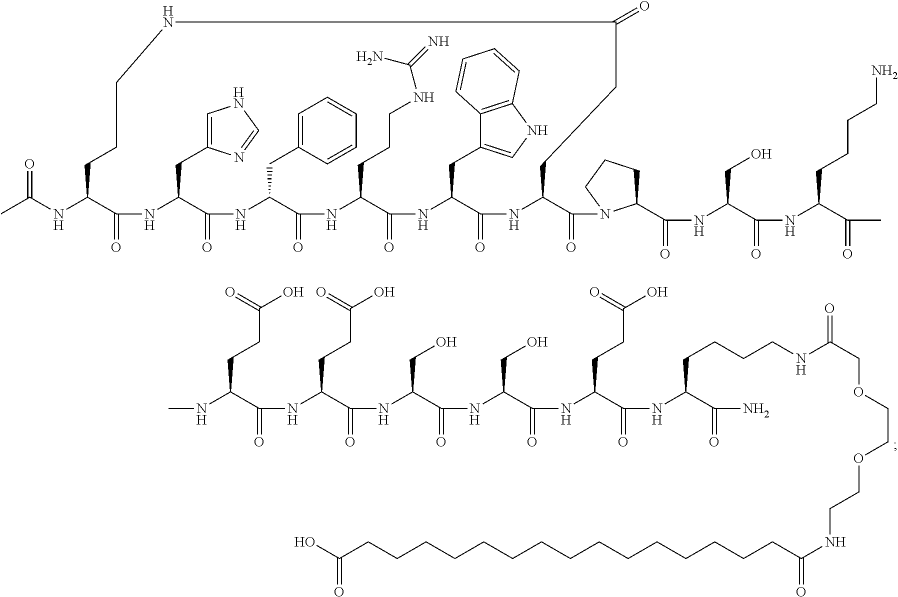
Ac-c[Lys-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 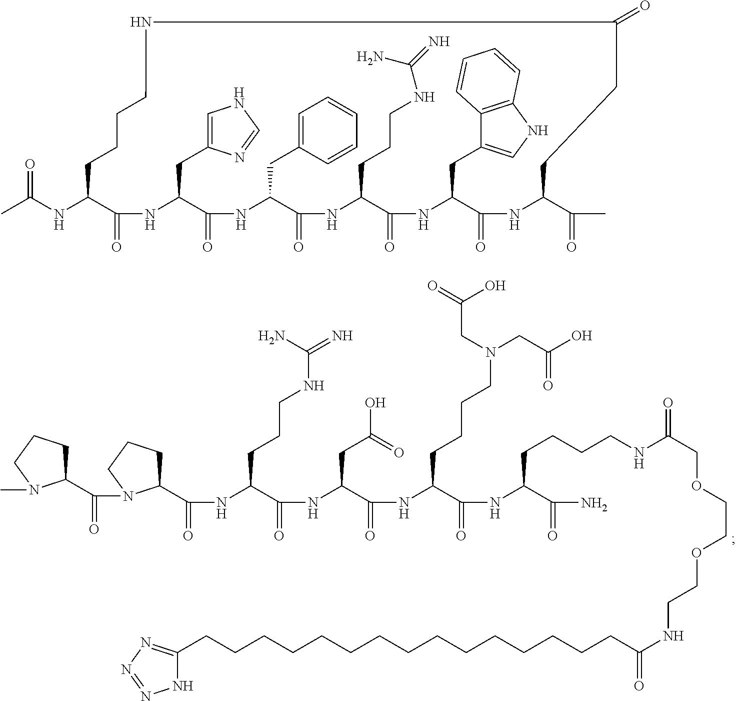
Ac-c[Orn-His-D-Phe-Arg-Trp-Glu]-Pro-Pro-His-Glu-Lys(biscarboxymethyl)-Lys({2-[2-(16-carboxyhexadecanoylamino)ethoxy]ethoxy}acetyl)-NH₂ 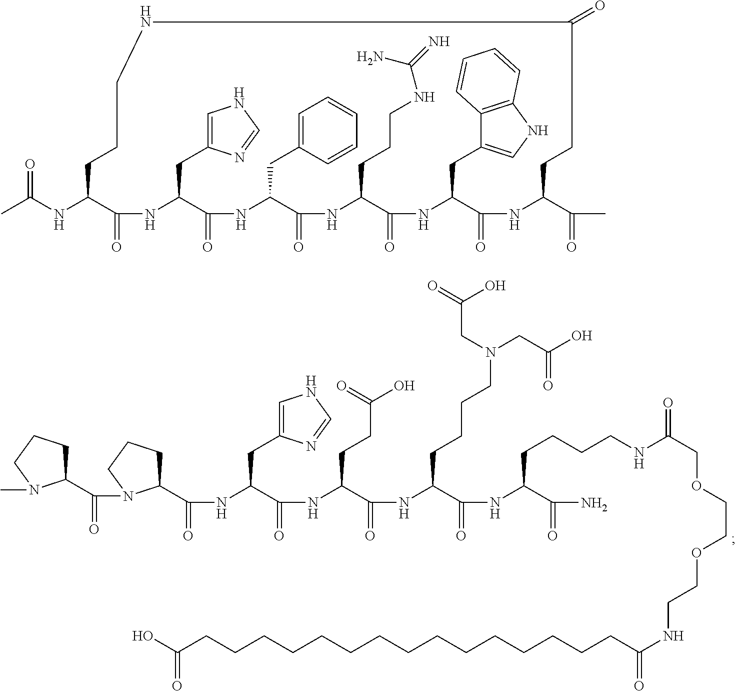
Ac-c[Dab-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 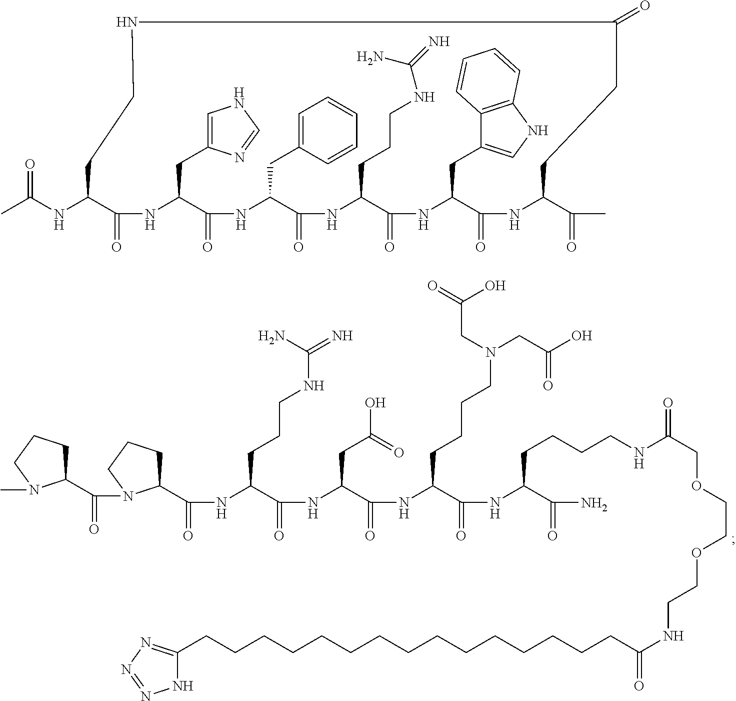
Ac-c[Asp-His-D-Phe-Arg-Trp-Dap]-Pro-Pro-Arg-Asp-Lys(BCMA)-Lys[(2-{2-[16-(tetrazol-5-yl)hexadecanoylamino]ethoxy}ethoxy)acetyl]-NH₂ 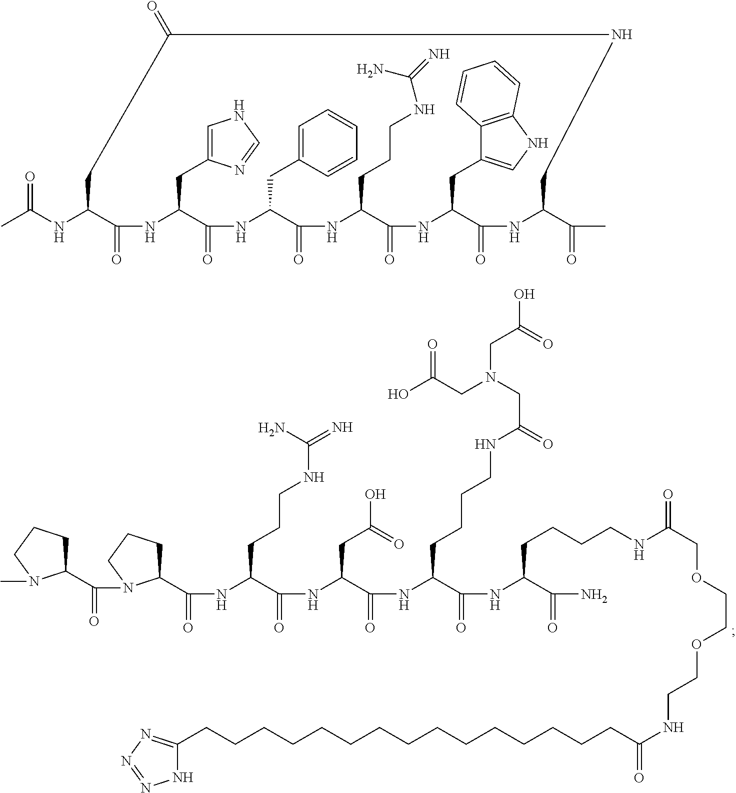 [SMILES: CC(=O)N[C@H]1CC(=O)NC[C@@H](C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN1CCC[C@H]1C(=O)N1CCC[C@H]1C(=O)N[C@@H](CCCNC(=N)N)C(=O)N[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Dab-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 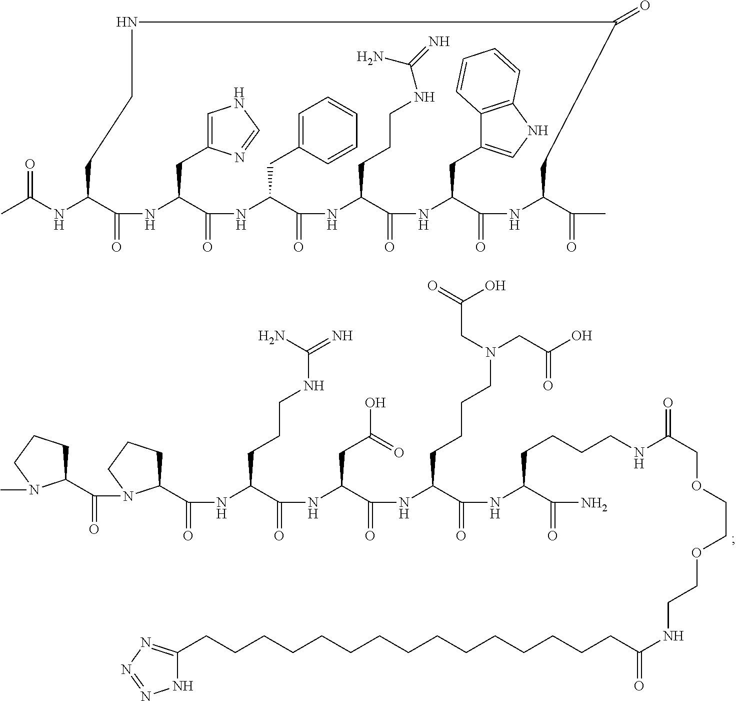
Ac-c[Glu-His-D-Phe-Arg-Trp-Orn]-Pro-Pro-Asp-Lys(biscarboxymethyl)-Lys({2-[2-(17-carboxyheptadecanoylamino)ethoxy]ethoxy}acetyl)-NH₂ 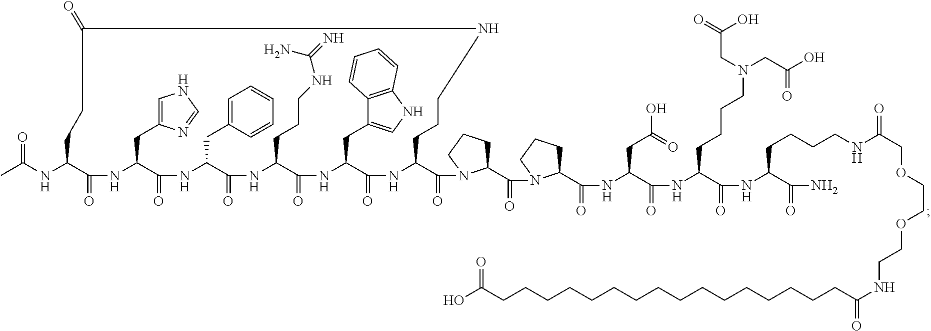 [SMILES: CC(=O)N[C@H]1CCC(=O)NCCC[C@@H](C(=O)N2CCC[C@H]2C(=O)N2CCC[C@H]2C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O]
Ac-c[Glu-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 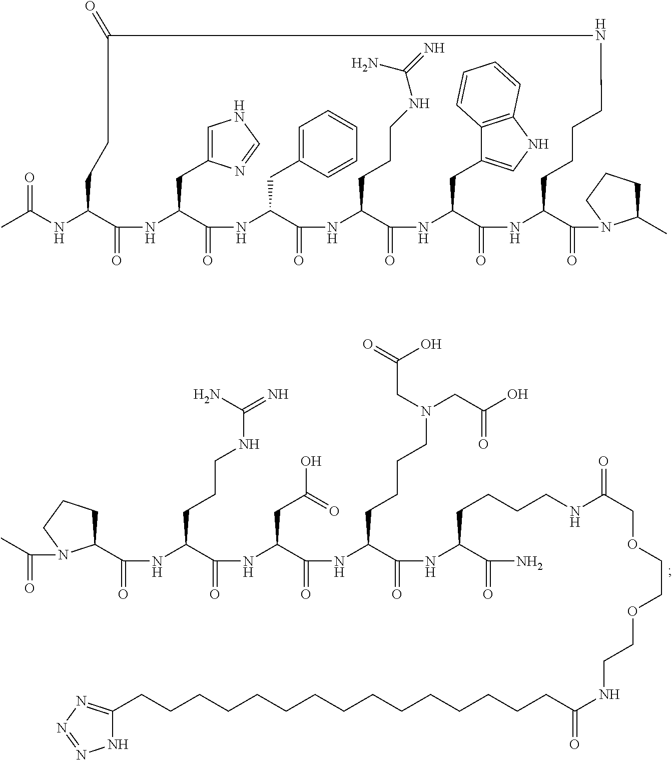
Ac-c[Orn-His-D-Phe-Arg-Trp-Asp]-Pro-Ser-Ser-Ser-Lys(biscarboxymethyl)-Ser-Ser-Lys[(2-{2-[16-(tetrazol-5-yl)hexadecanoylamino]ethoxy}ethoxy)acetyl]-NH₂ 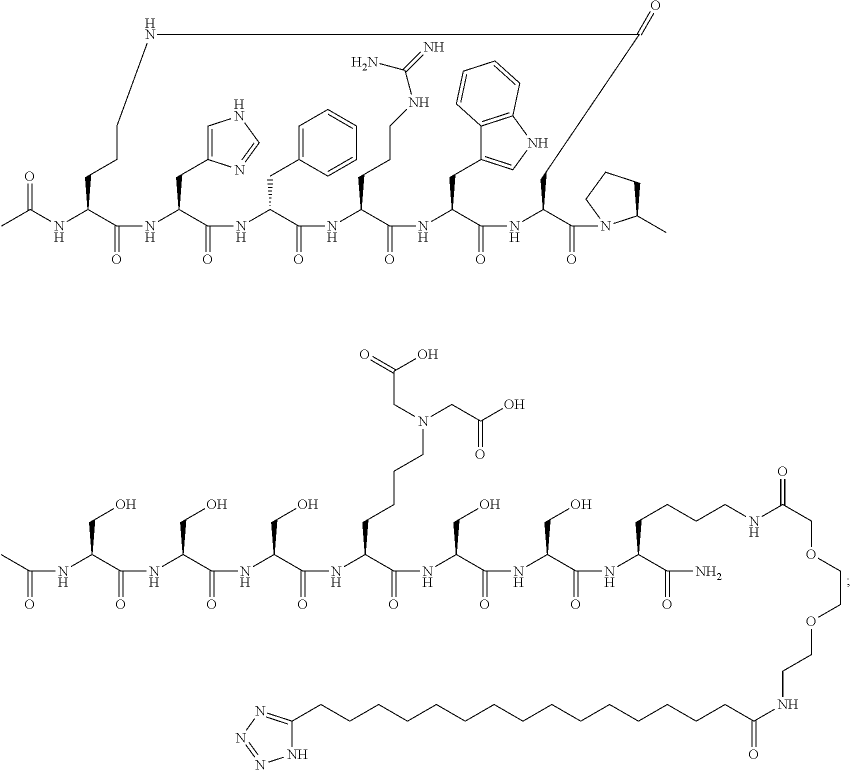
Ac-c[Glu-His-D-Phe-Arg-Trp-Orn]-D-Pro-D-Pro-Arg-Asp-Lys(biscarboxymethyl)-Lys[(2-{2-[16-(tetrazol-5-yl)hexadecanoylamino]ethoxy}ethoxy)acetyl]-NH₂ 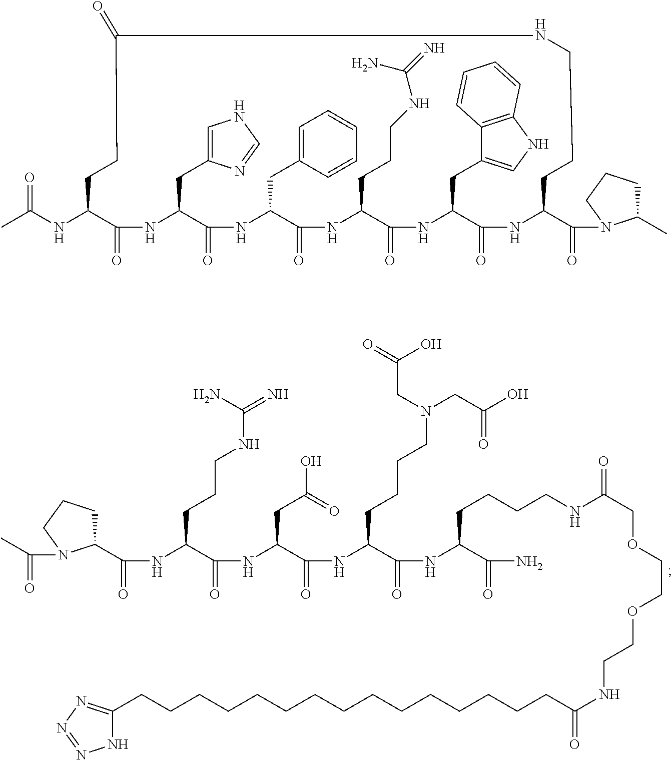
Ac-c[Asp-His-D-Phe-Arg-Trp-Dab]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 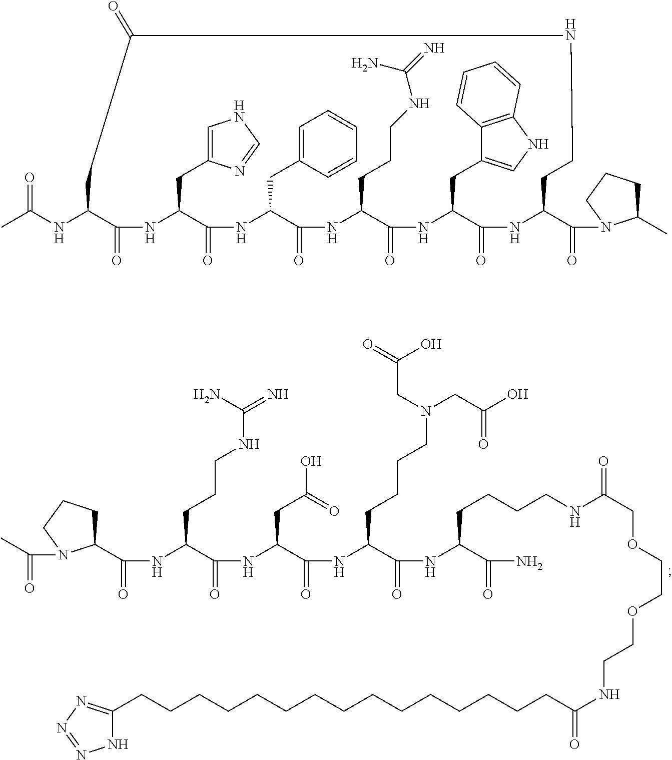
Ac-c[Glu-His-D-Phe-Arg-Trp-Dap]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂ 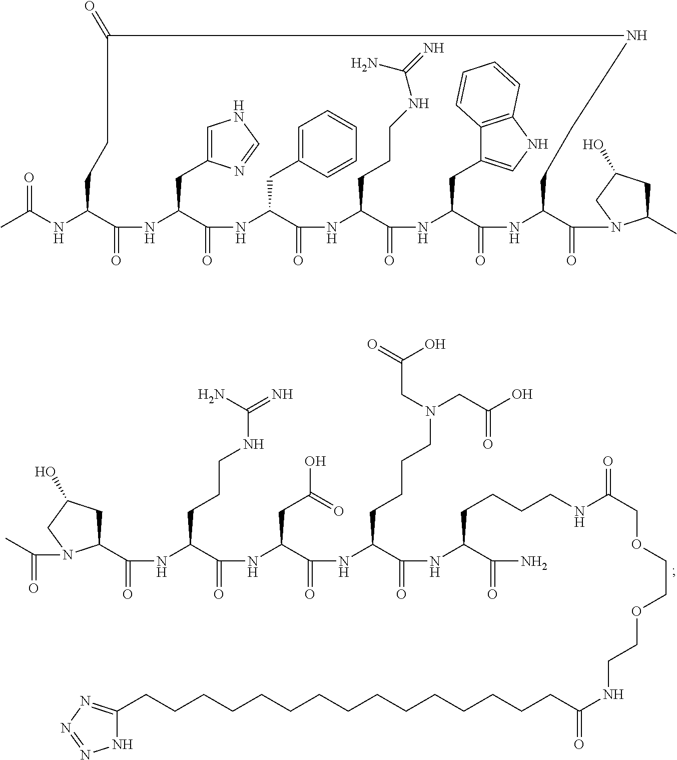
Ac-c[Glu-His-D-Phe-Arg-Trp-Dab]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂ 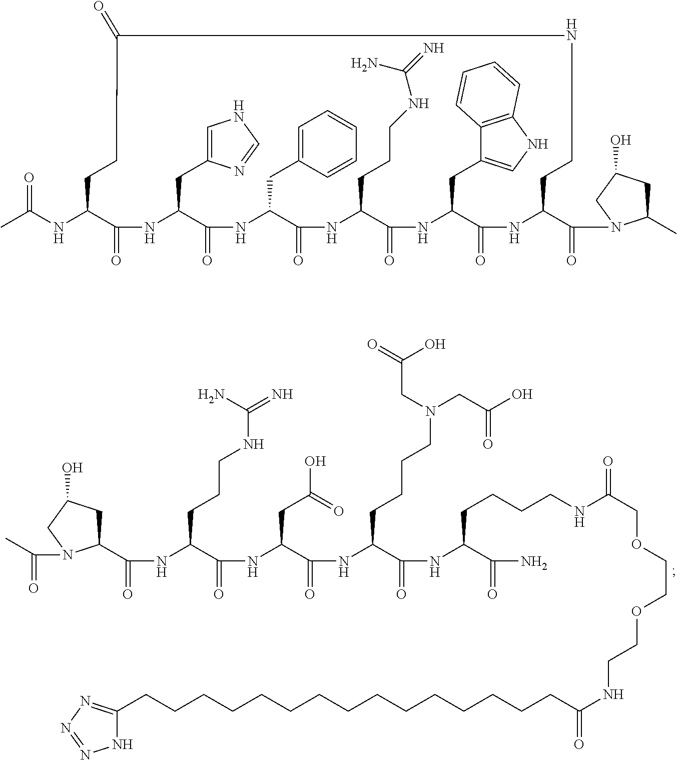
Ac-c[Dab-His-D-Phe-Arg-Trp-Asp]-Pro-Pro-His-Glu-Lys(biscarboxymethyl)-Lys[(2-{2-[16-(tetrazol-5-yl)hexadecanoylamino]ethoxy}ethoxy)acetyl]-NH₂ 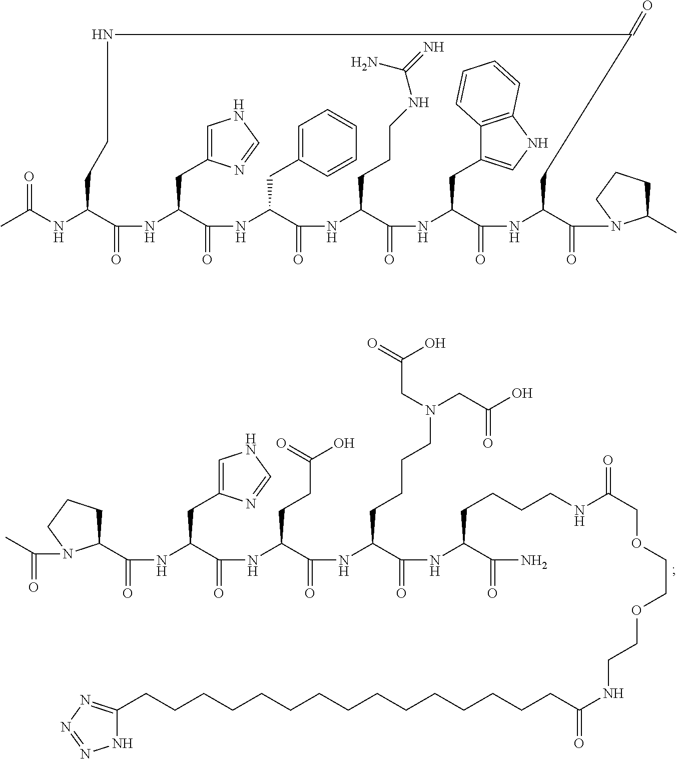
Ac-c[Orn-His-D-Phe-Arg-Trp-Asp]-Pro-Pro-Arg-Glu-Lys(biscarboxymethyl)-Lys({2-[2-(17-carboxyheptadecanoylamino)ethoxy]ethoxy}acetyl)-NH₂ 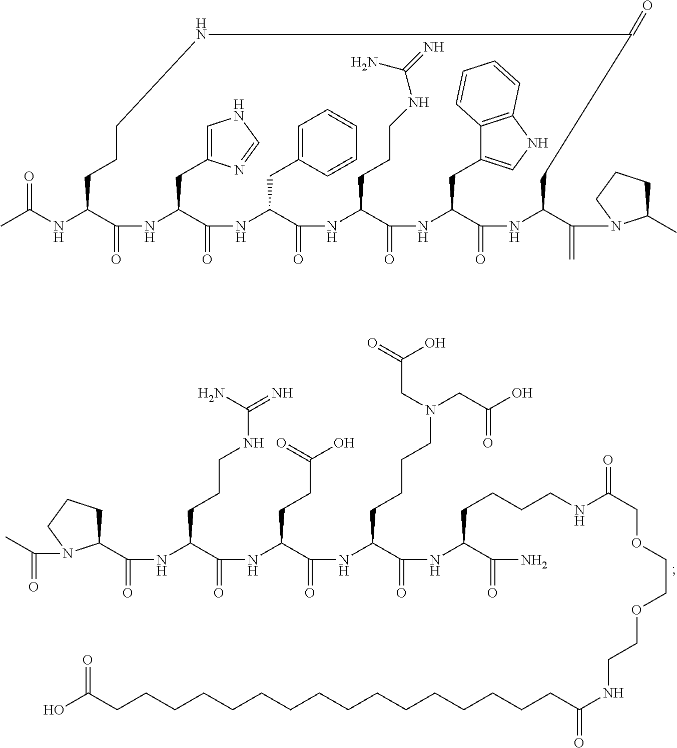
Ac-c[Asp-His-D-Phe-Arg-Trp-Lys]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 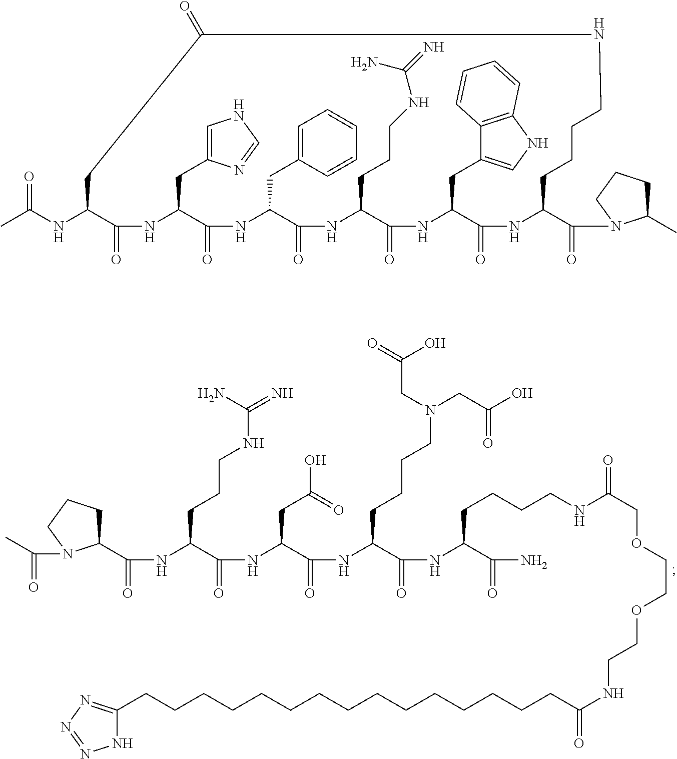
Ac-c[Dap-His-D-Phe-Arg-Trp-Asp]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 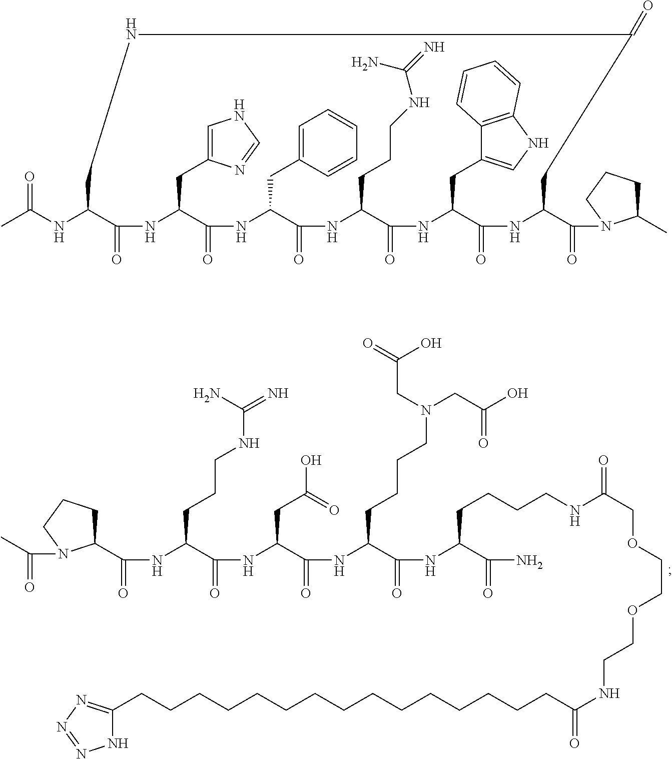
Ac-c[Glu-Gln-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 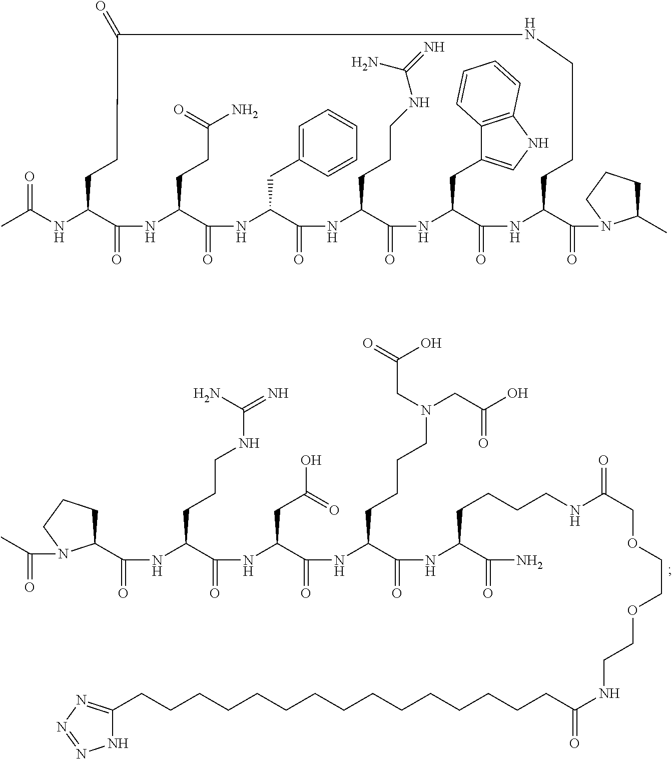
Ac-c[Glu-His-D-Phe-Arg-Trp-Orn]-Pro-Pro-Arg-Asp-Lys(biscarboxymethyl)-Lys({2-[2-(17-carboxyheptadecanoylamino)ethoxy]ethoxy}acetyl)-NH₂ 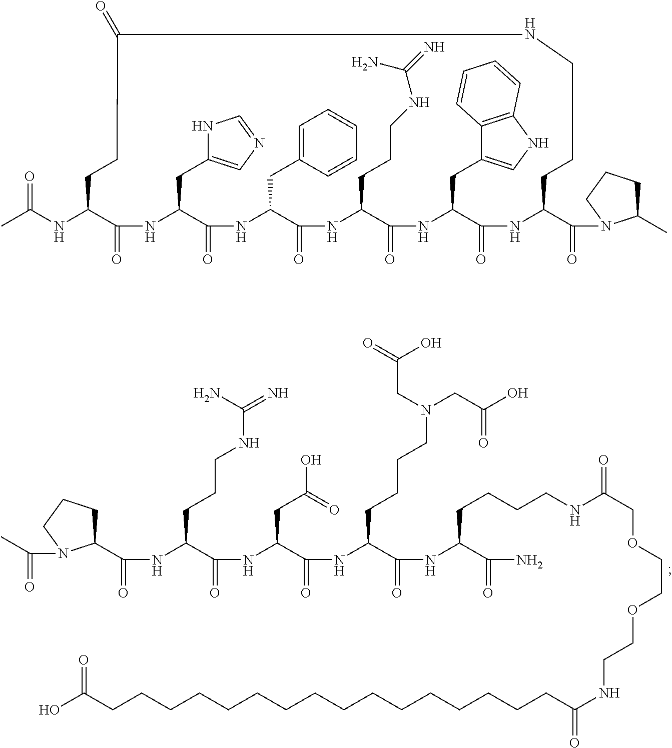
Ac-c[Orn-His-D-Phe-Arg-Trp-Glu]-Pro-Ser-Ser-Ser-Glu-Glu-Ser-Ser-Lys({2-[2-(16-carboxyhexadecanoylamino)ethoxy]ethoxy}acetyl)-NH₂ 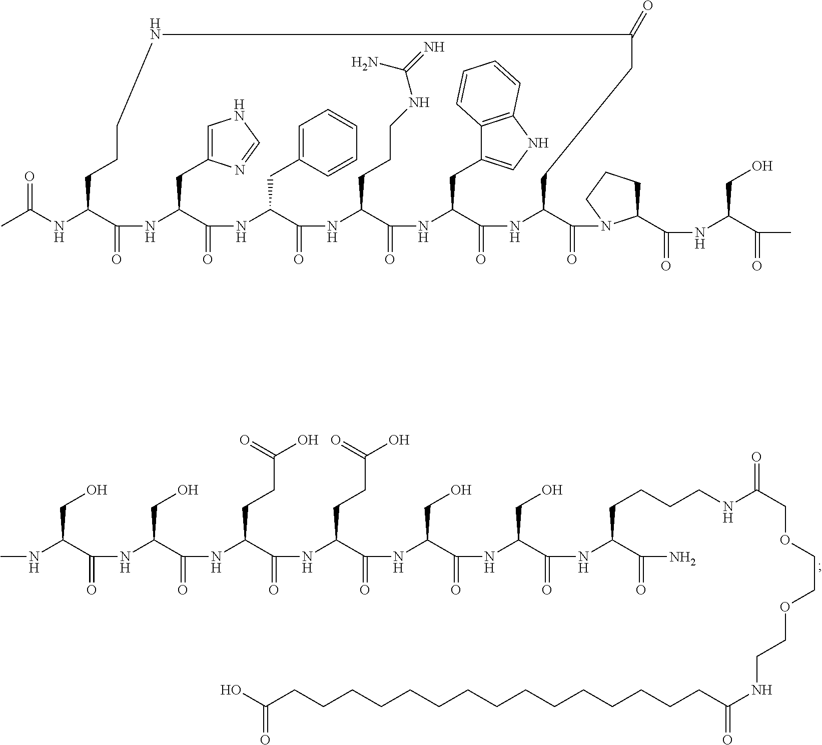
Ac-c[Orn-Glu-His-D-Phe-Arg-Trp-Gly-Asp]-Pro-Ser-Lys-Glu-Glu-Ser-Ser-Lys({2-[2-(16-carboxyhexadecanoylamino)ethoxy]ethoxy}acetyl)-NH₂ 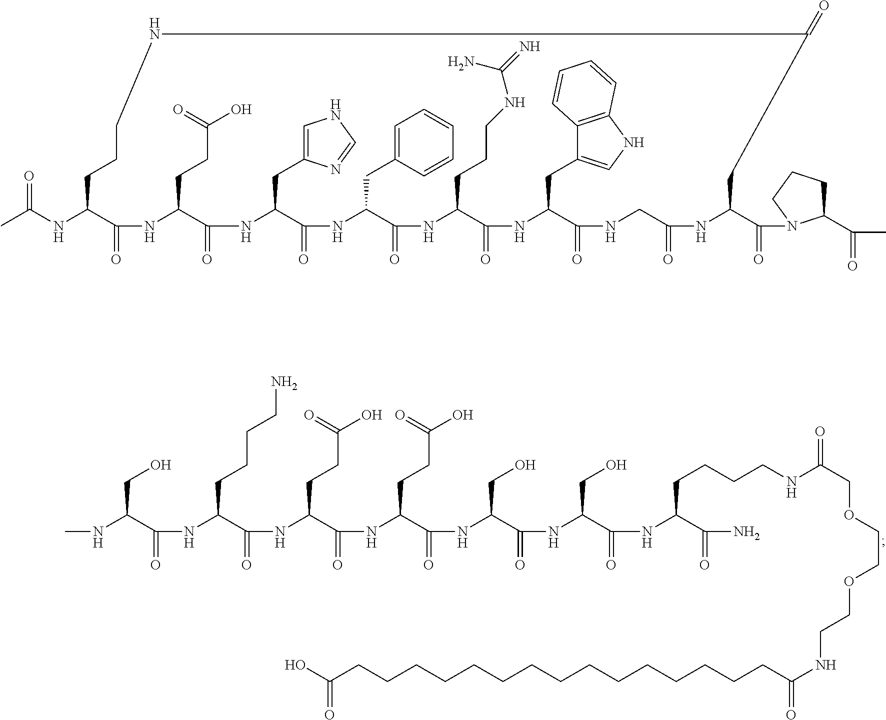
Ac-c[Asp-His-D-Phe-Arg-Trp-Dap]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 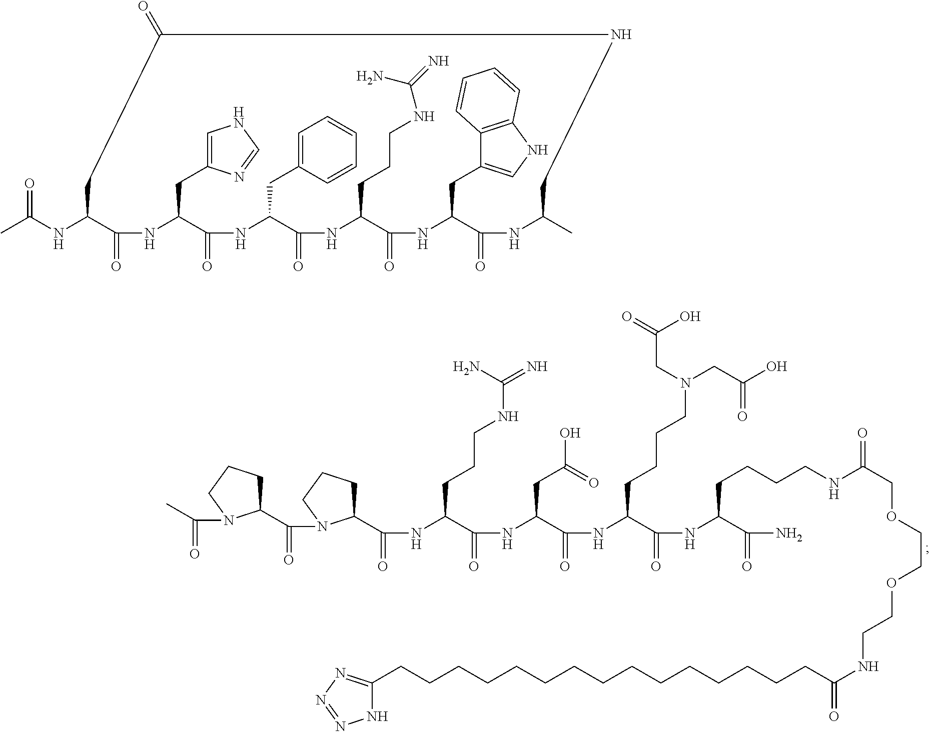 [SMILES: CC(=O)N[C@H]1CC(=O)NC[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Orn-His-D-Phe-Arg-Trp-Asp]-Pro-Pro-Arg-Glu-Lys(biscarboxymethyl)-Lys({2-[2-(15-carboxypentadecanoylamino)ethoxy]ethoxy}acetyl)-NH₂ 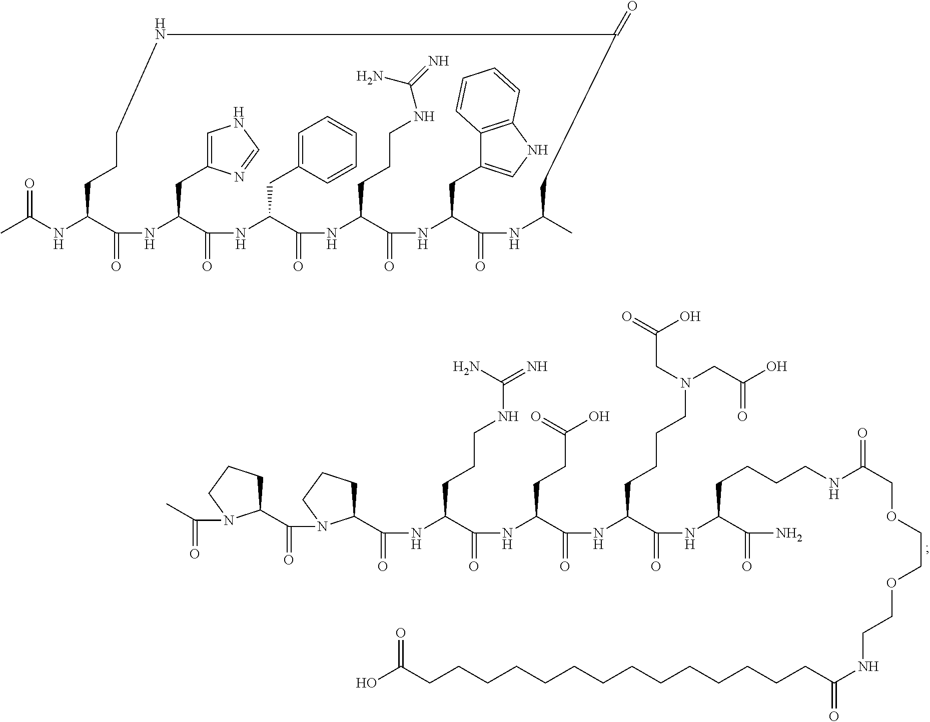 [SMILES: CC(=O)N[C@H]1CCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]
Ac-c[Glu-His-D-Phe-Arg-Trp-Orn]-Pro-Pro-His-Asp-Lys(biscarboxymethyl)-Lys[(2-{2-[16-(tetrazol-5-yl)hexadecanoylamino]ethoxy}ethoxy)acetyl]-NH₂ 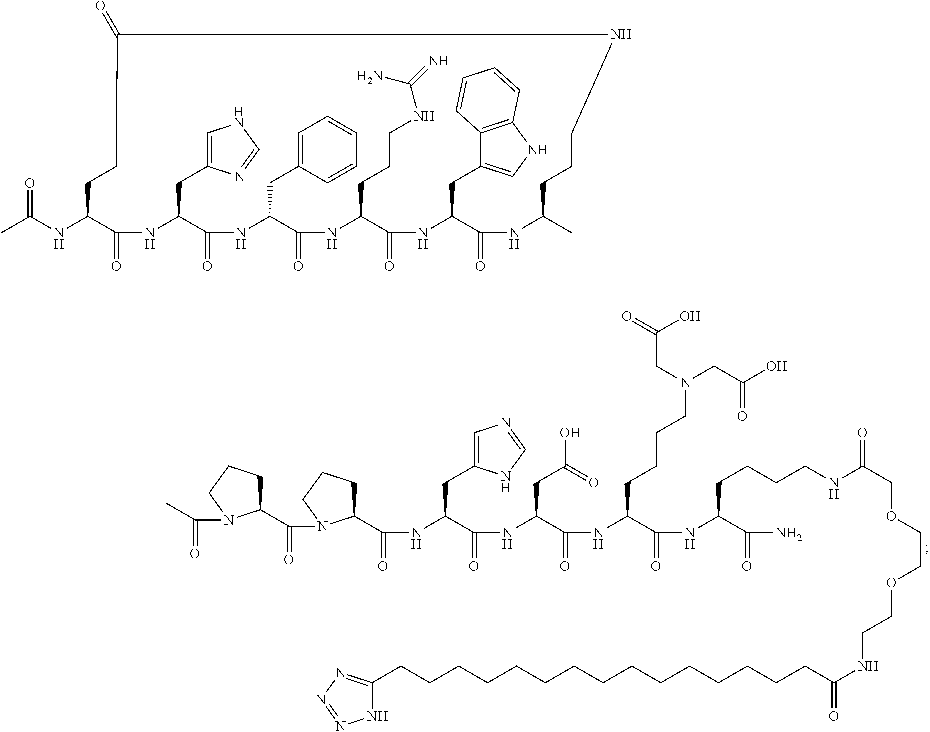 [SMILES: CC(=O)N[C@H]1CCC(=O)NCCC[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](Cc1cnc[nH]1)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Lys-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂ 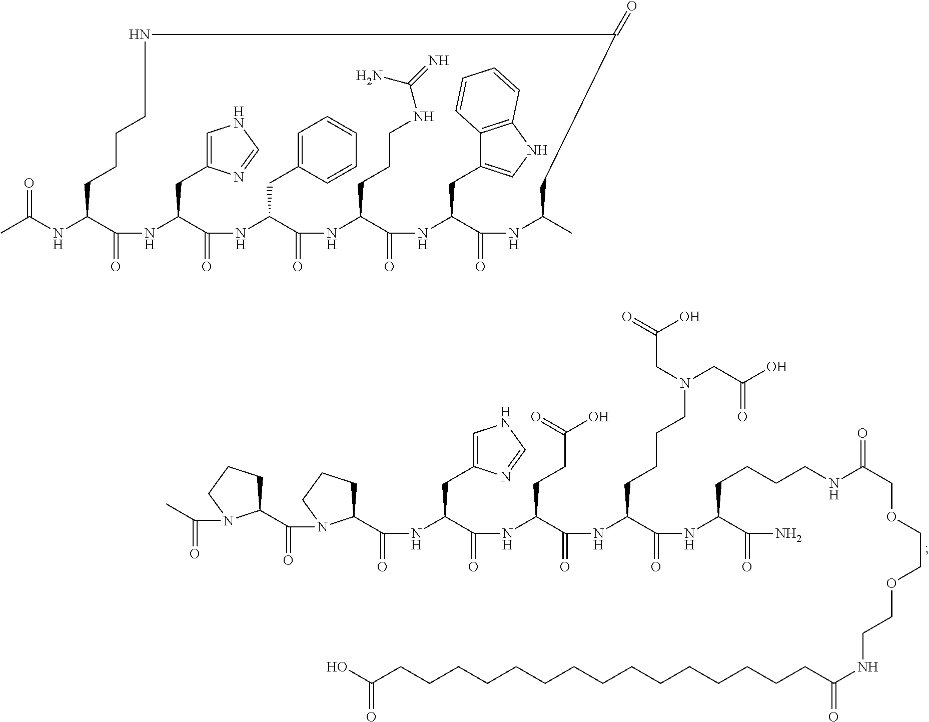 [SMILES: CC(=O)N[C@H]1CCCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]
Ac-c[Lys-His-D-Phe-Arg-Trp-Asp]-Pro-Pro-His-Ser-Lys(biscarboxymethyl)-Ser-Ser-Glu-Lys({2-[2(16-carboxyhexadecanoylamino)ethoxy]ethoxy}acetyl)-NH₂ 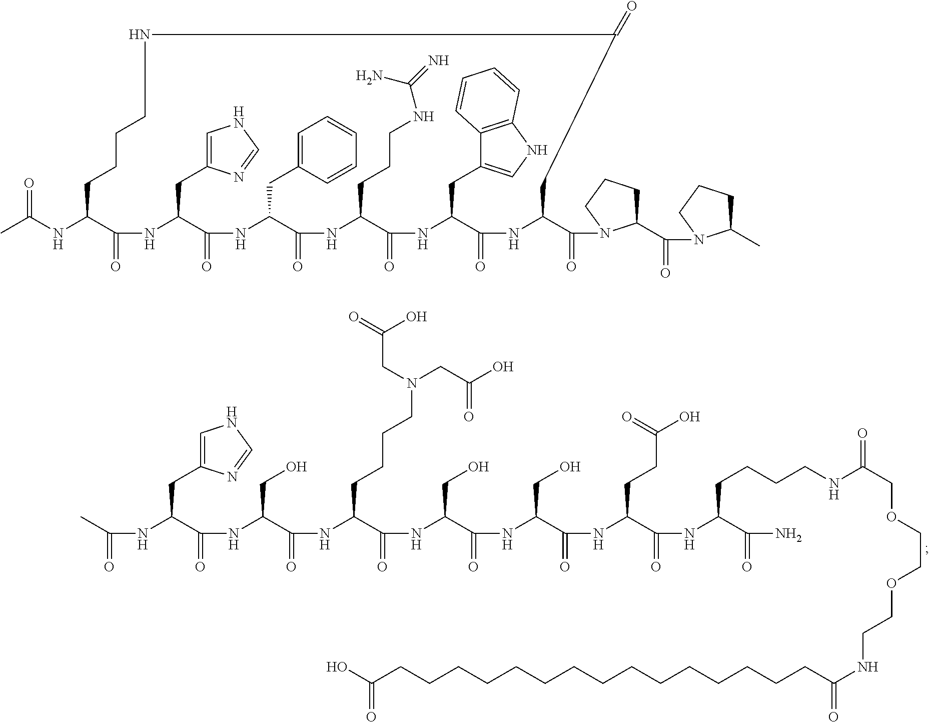
Ac-c[Orn-His-D-Phe-Arg-Trp-Asp]-Pro-Pro-His-Glu-Lys(biscarboxymethyl)-Lys({2-[2-(17-carboxyheptadecanoylamino)ethoxy]ethoxy}acetyl)-NH₂ 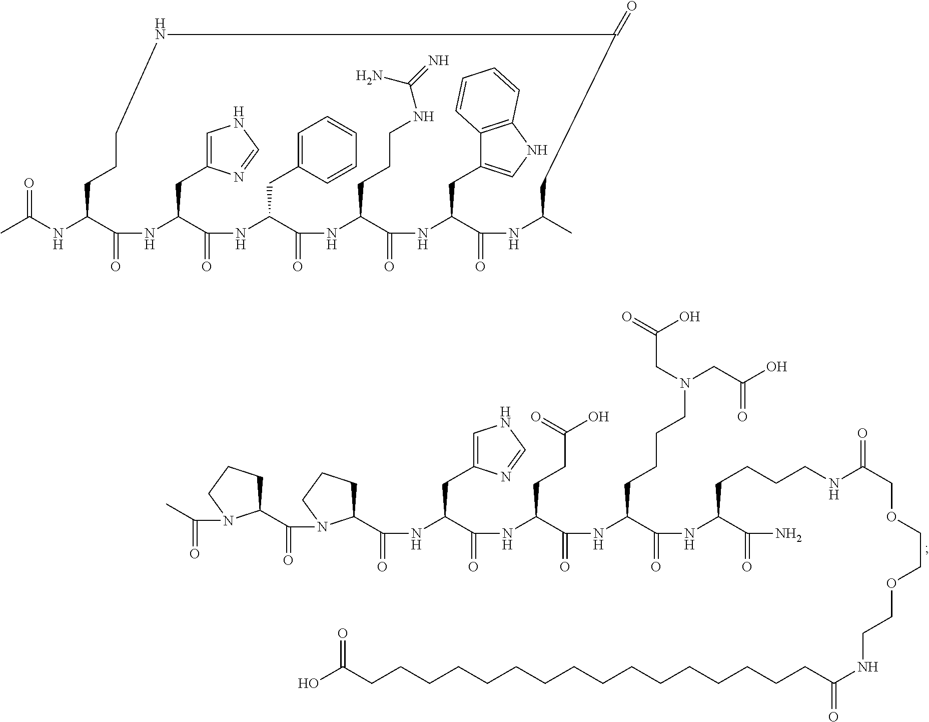 [SMILES: CC(=O)N[C@H]1CCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]
Ac-c[Dap-His-D-Phe-Arg-Trp-Glu]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 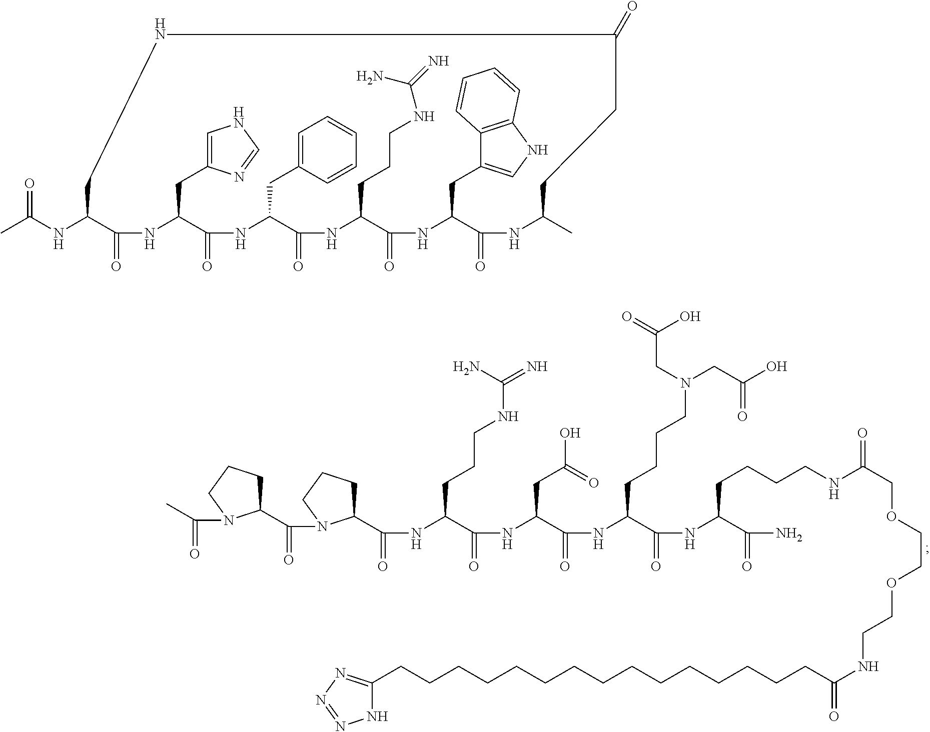 [SMILES: CC(=O)N[C@H]1CNC(=O)CC[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Glu-His-D-Phe-Arg-Trp-Lys]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂ 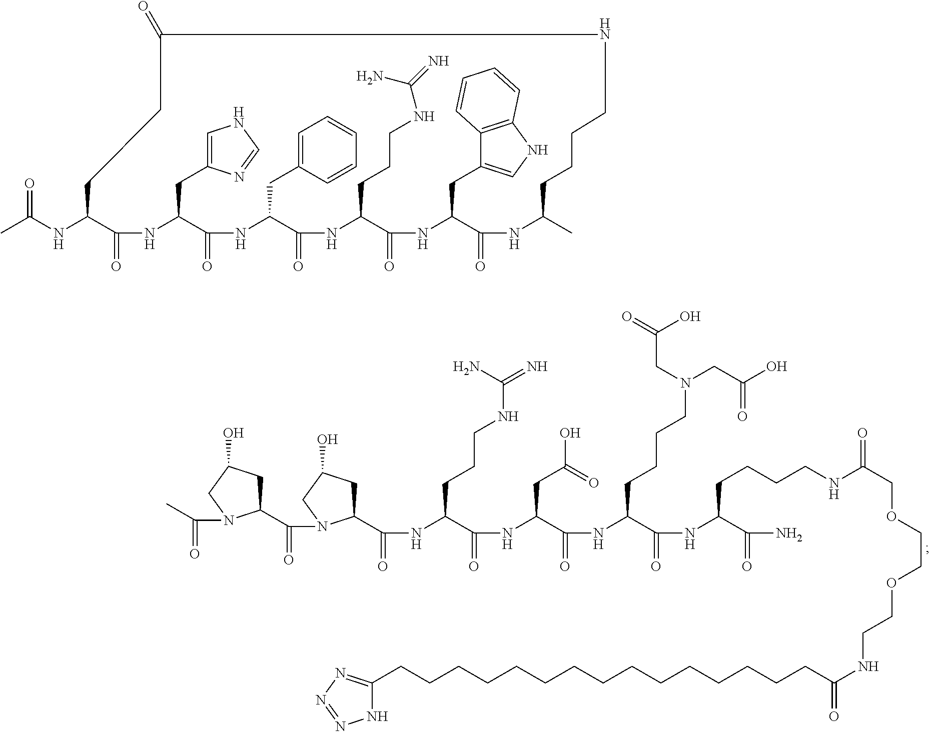 [SMILES: CC(=O)N[C@H]1CCC(=O)NCCCC[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1C[C@H](O)C[C@H]1C(=O)N1C[C@H](O)C[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Lys-His-D-Phe-Arg-Trp-Asp]-Pro-Ser-His-Glu-Lys(biscarboxymethyl)-Lys({2-[2-(16-carboxyhexadecanoylamino)ethoxy]ethoxy}acetyl)-NH₂ 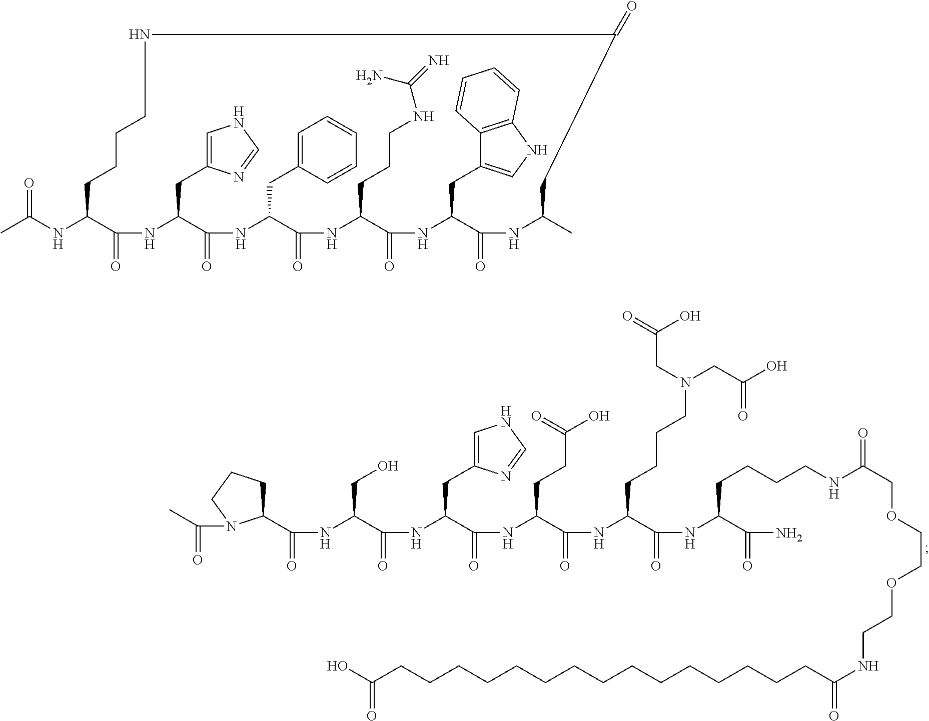 [SMILES: CC(=O)N[C@H]1CCCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N[C@@H](CO)C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]
Ac-c[Orn-His-D-Phe-Arg-Trp-Asp]-Pro-Pro-Arg-Glu-Lys(biscarboxymethyl)-Ser-Ser-NH(2-{2-[2-(17-carboxyheptadecanoylamino)ethoxy]ethoxy}ethyl) 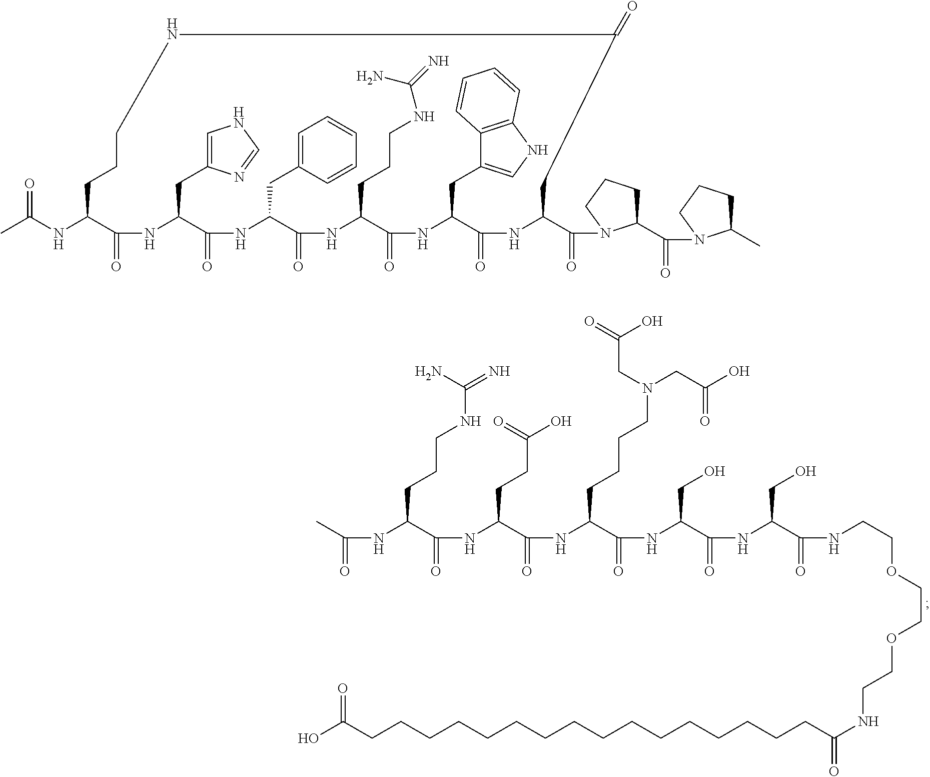 [SMILES: CC(=O)N[C@H]1CCCNC(=O)C[C@@H](C(=O)N2CCC[C@H]2C(=O)N2CCC[C@H]2C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N[C@@H](CCCNC(=N)N)C(=O)N[C@@H](CCC(=O)O)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]
Ac-c[Orn-His-D-Phe-Arg-Trp-Asp]-Pro-Pro-His-Glu-Lys(biscarboxymethyl)-Lys({2-[2-(15-carboxypentadecanoylamino)ethoxy]ethoxy}acetyl)-NH₂ 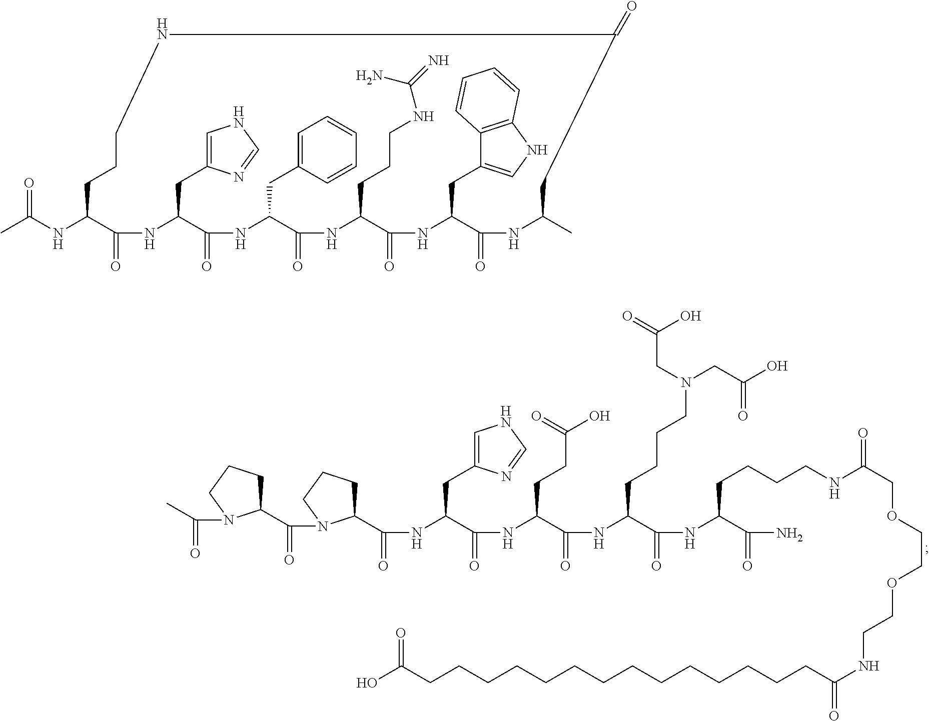 [SMILES: CC(=O)N[C@H]1CCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]
Ac-c[Lys-His-D-Phe-Arg-Trp-Asp]-Pro-Pro-His-Glu-Glu-Ser-Ser-Glu-Lys({2-[2-(16-carboxyhexadecanoylamino)ethoxy]ethoxy}acetyl)-NH₂ 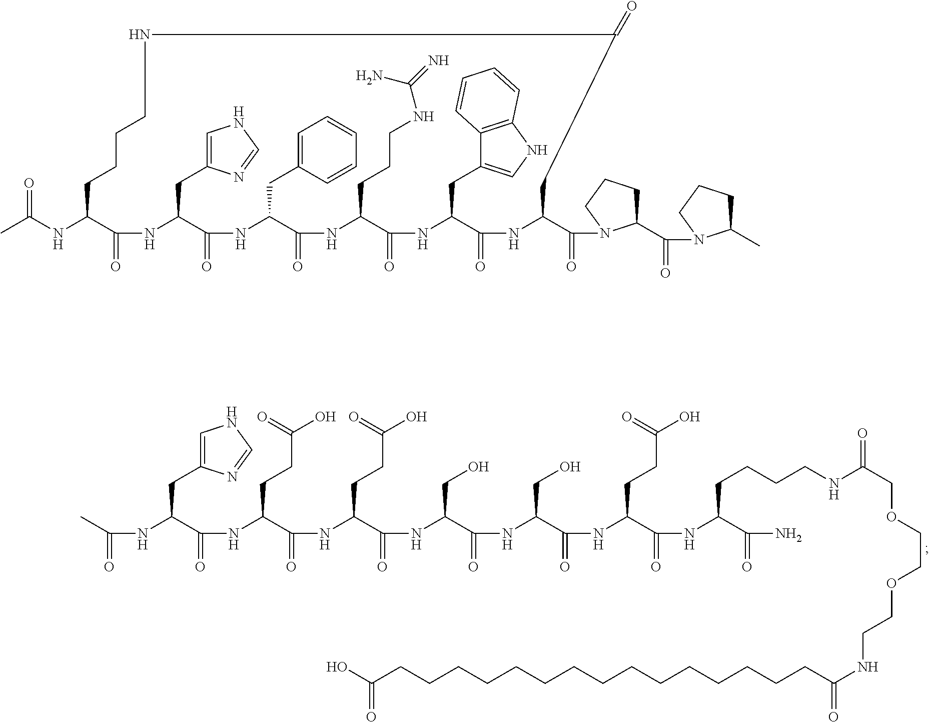
Ac-c[Lys-His-D-Phe-Arg-Trp-Asp]-Pro-Ser-Ser-Ser-Lys(biscarboxymethyl)-Ser-Ser-NH(2-{2-[2-(17-carboxyheptadecanoylamino)ethoxy]ethoxy}ethyl) 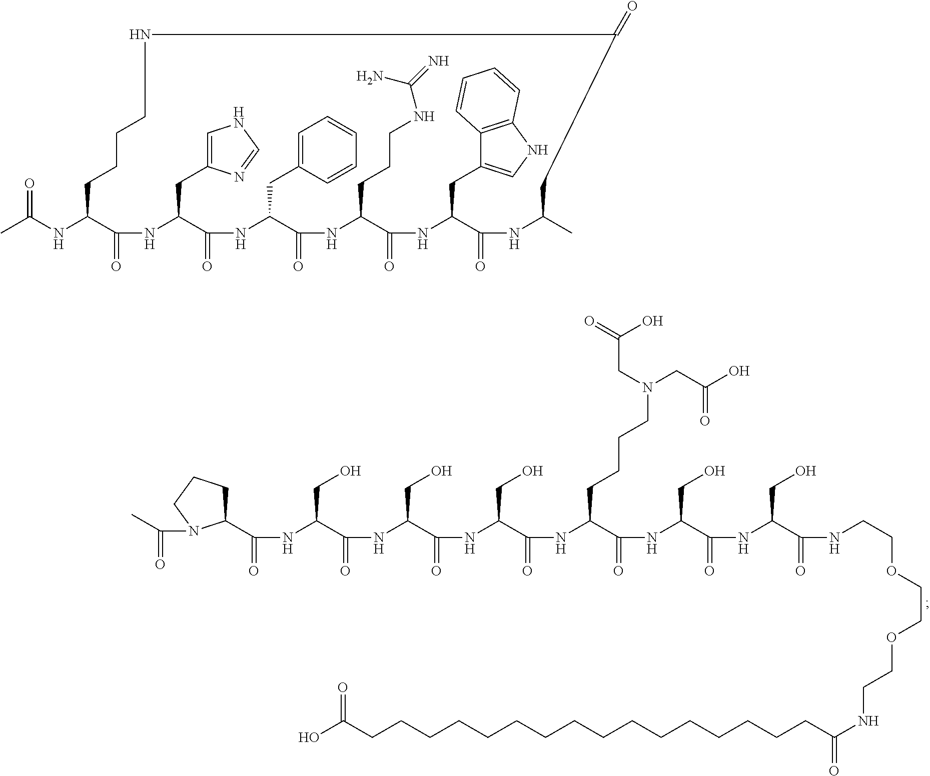 [SMILES: CC(=O)N[C@H]1CCCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N[C@@H](CO)C(=O)N[C@@H](CO)C(=O)N[C@@H](CO)C(=O)N[C@@H](CCCCN(CC(=O)O)CC(=O)O)C(=O)N[C@@H](CO)C(=O)N[C@@H](CO)C(=O)NCCOCCOCCNC(=O)CCCCCCCCCCCCCCCCC(=O)O]
Ac-c[Glu-His-D-Phe-Arg-Trp-Orn]-Pro-Pro-Arg-Asp-Lys(biscarboxymethyl)-Lys[(2-{2-[16-(tetrazol-5-yl)hexadecanoylamino]ethoxy}ethoxy)acetyl]-NH₂ 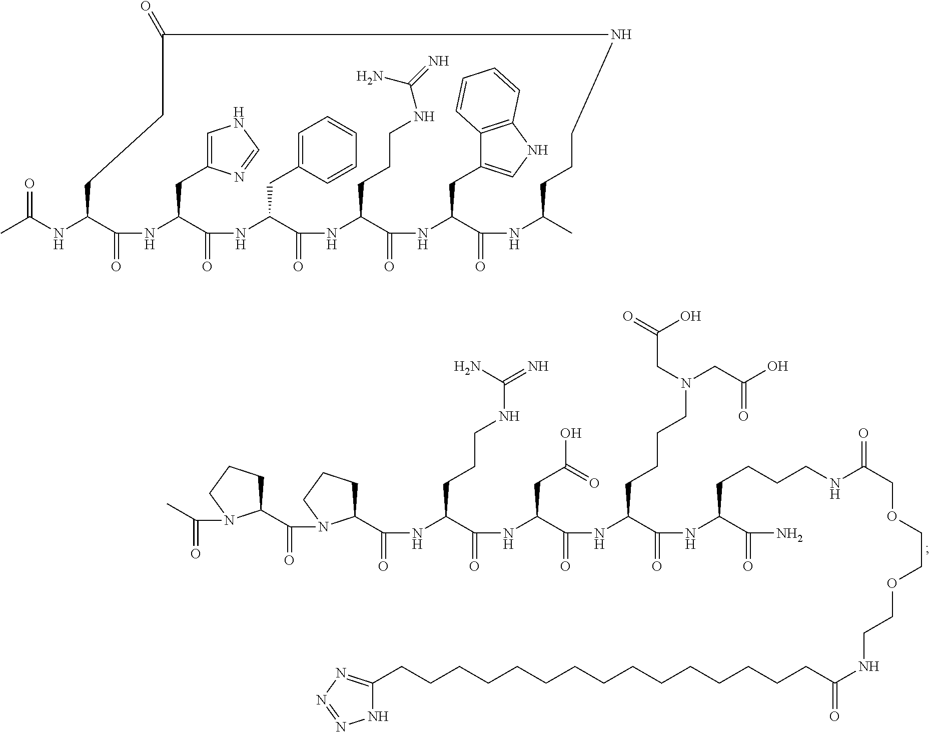 [SMILES: CC(=O)N[C@H]1CCC(=O)NCCC[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Orn-His-D-Phe-Arg-Trp-Asp]-Pro-Pro-His-Glu-Lys(biscarboxymethyl)-Lys({2-[2-(16-carboxyhexadecanoylamino)ethoxy]ethoxy}acetyl)-NH₂ 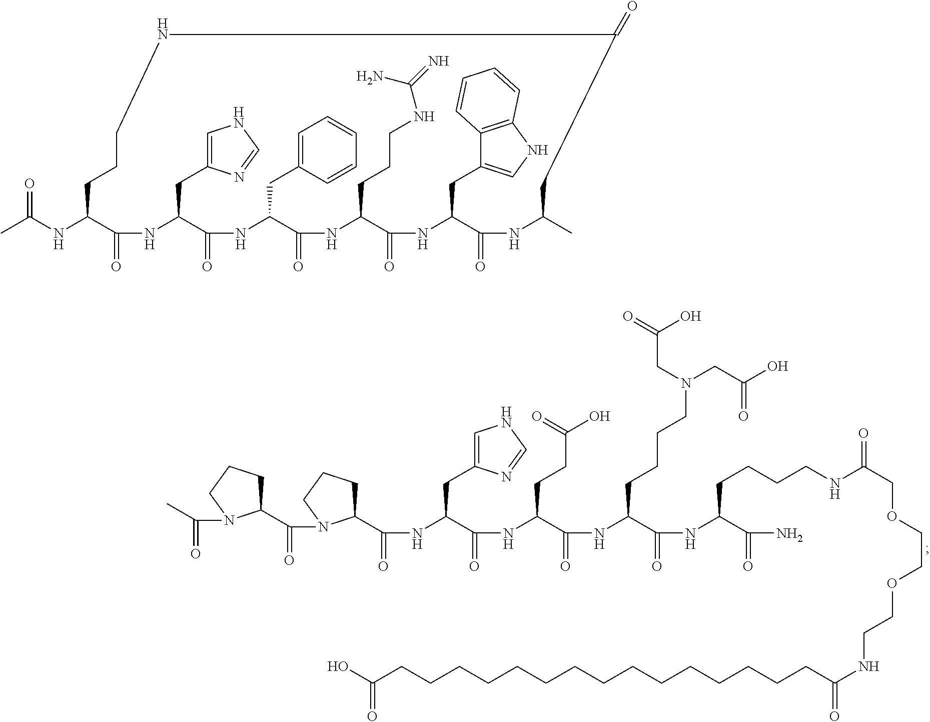 [SMILES: CC(=O)N[C@H]1CCCNC(=O)C[C@@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CC(=O)N1CCC[C@H]1C(=O)N1CCC[C@H]1C(=O)N[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCC(=O)O)C(N)=O]
Ac-c[Glu-His-D-Phe-Arg-Trp-Orn]-Hyp-Hyp-Arg-Asp-Lys(biscarboxymethyl)-Lys[(2-{2-[16-(tetrazol-5-yl)hexadecanoylamino]ethoxy}ethoxy)acetyl]-NH₂ 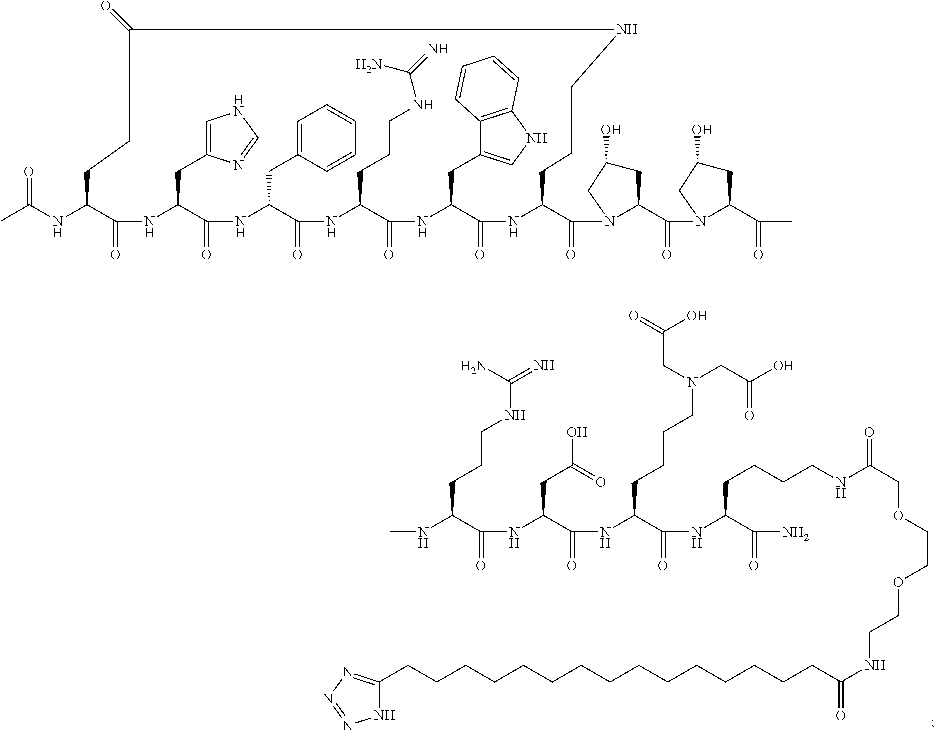 [SMILES: CC(=O)N[C@H]1CCC(=O)NCCC[C@@H](C(=O)N2C[C@H](O)C[C@H]2C(=O)N2C[C@H](O)C[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCNC(=N)N)C(=O)N[C@@H](CC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Orn-His-D-Phe-Arg-Trp-Glu]-Pro-Ser-Ser-Ser-Lys(biscarboxymethyl)-Ser-Ser-Lys({2-[2-(16-carboxyhexadecanoylamino)ethoxy]ethoxy}acetyl)-NH₂ 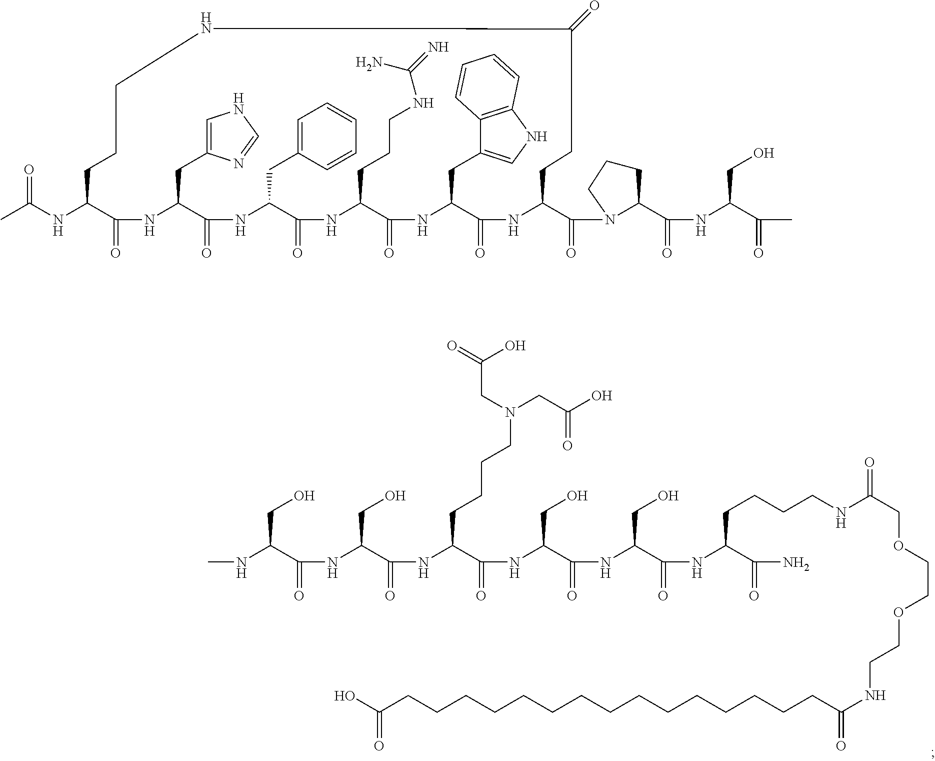
Ac-c[homoCys-Ser-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂ 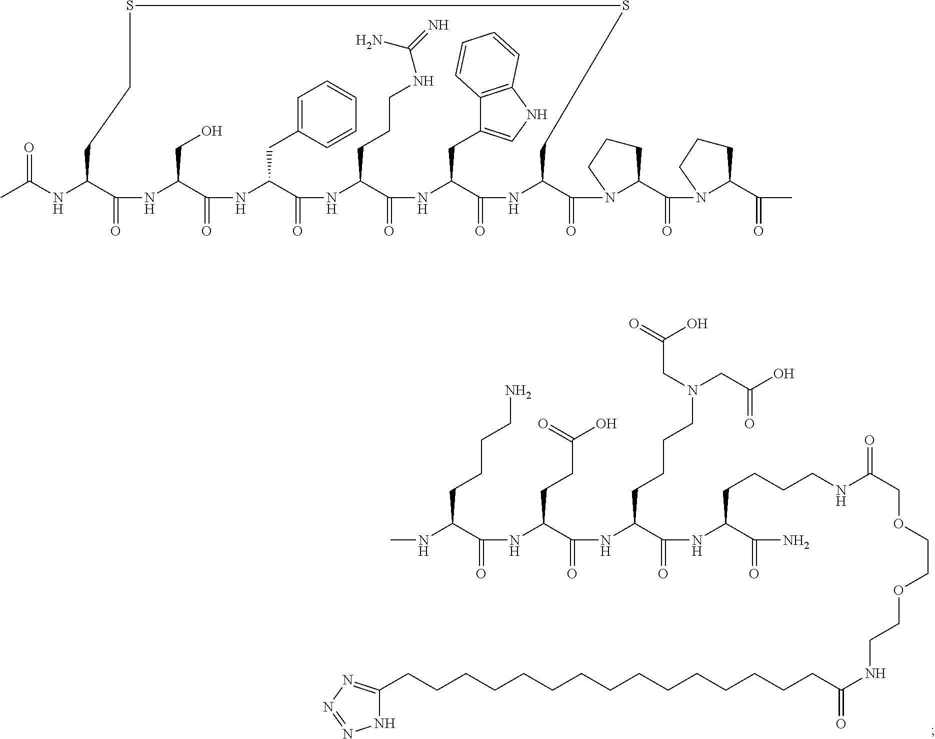 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](CO)NC1=O ; CN[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Cys-Glu-His-D-Phe-Arg-Trp-Gly-Cys]-Pro-Pro-Lys-Glu-Lys(biscarboxymethyl)-Lys({2-[2-(15-carboxypentadecanoylamino)ethoxy]ethoxy}acetyl)-NH₂ 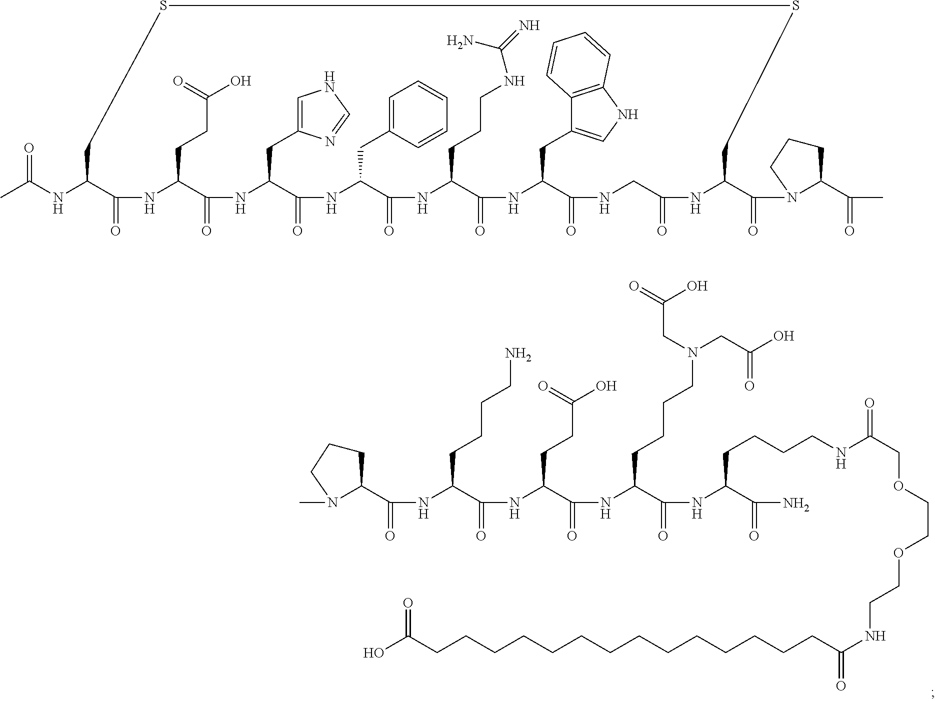
Ac-c[homoCys-His-D-Phe-Arg-Trp-Cys]-Ser-Pro-Lys-Glu-Lys(biscarboxymethyl)-Lys({2-[2-(15-carboxypentadecanoylamino)ethoxy]ethoxy}acetyl)-NH₂ 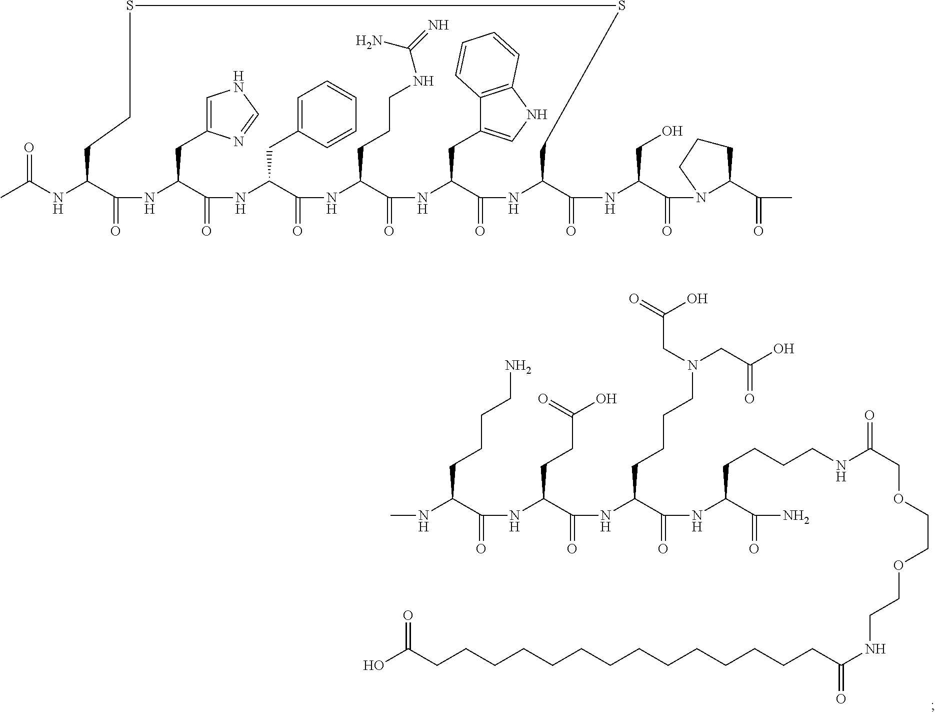 [SMILES: CC(=O)N[C@H]1CCSSC[C@@H](C(=O)N[C@@H](CO)C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]
Ac-c[homoCys-His-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂ 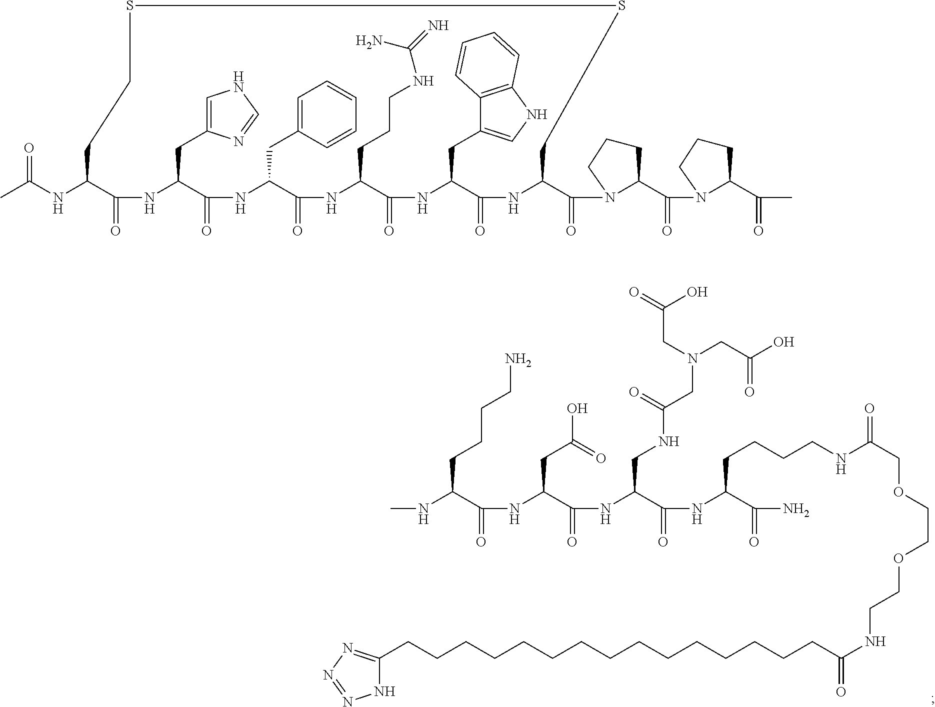 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[homoCys-His-D-Phe-Arg-Trp-Cys]-Ser-Pro-His-Glu-Lys(biscarboxymethyl)-Lys({2-[2-(17-carboxyheptadecanoylamino)ethoxy]ethoxy}acetyl)-NH₂ 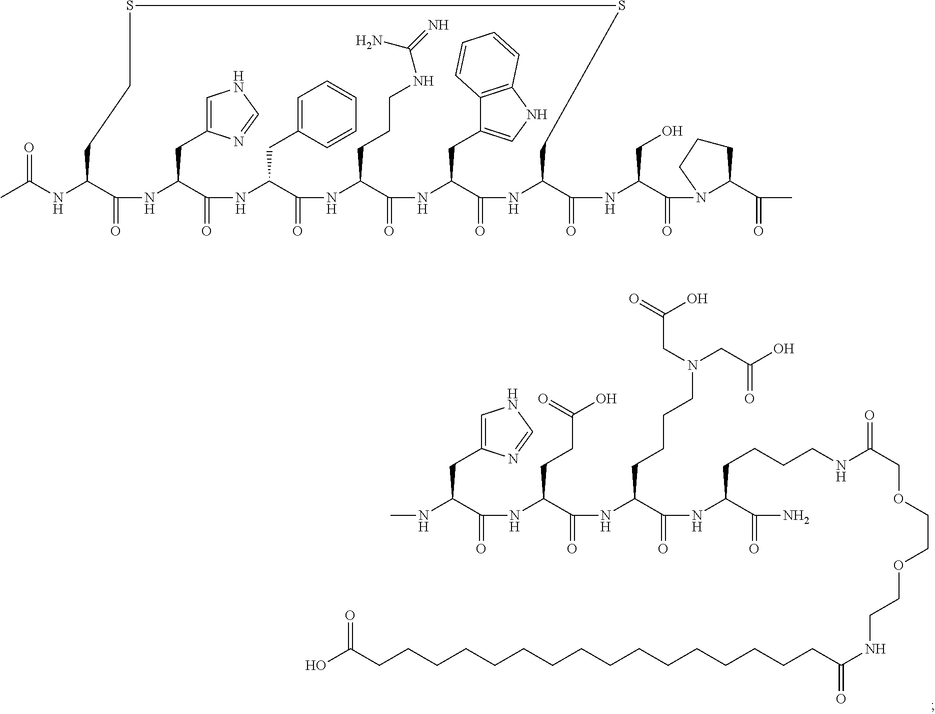 [SMILES: CC(=O)N[C@H]1CCSSC[C@@H](C(=O)N[C@@H](CO)C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](Cc1c[nH]cn1)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCCC(=O)O)C(N)=O]
Ac-c[Cys-Glu-His-D-Phe-Arg-Trp-Cys]-Pro-Pro-Lys-Glu-Lys(biscarboxymethyl)-Lys[(2-{2-[16-(tetrazol-5-yl)hexadecanoylamino]ethoxy}ethoxy)acetyl]-NH₂ 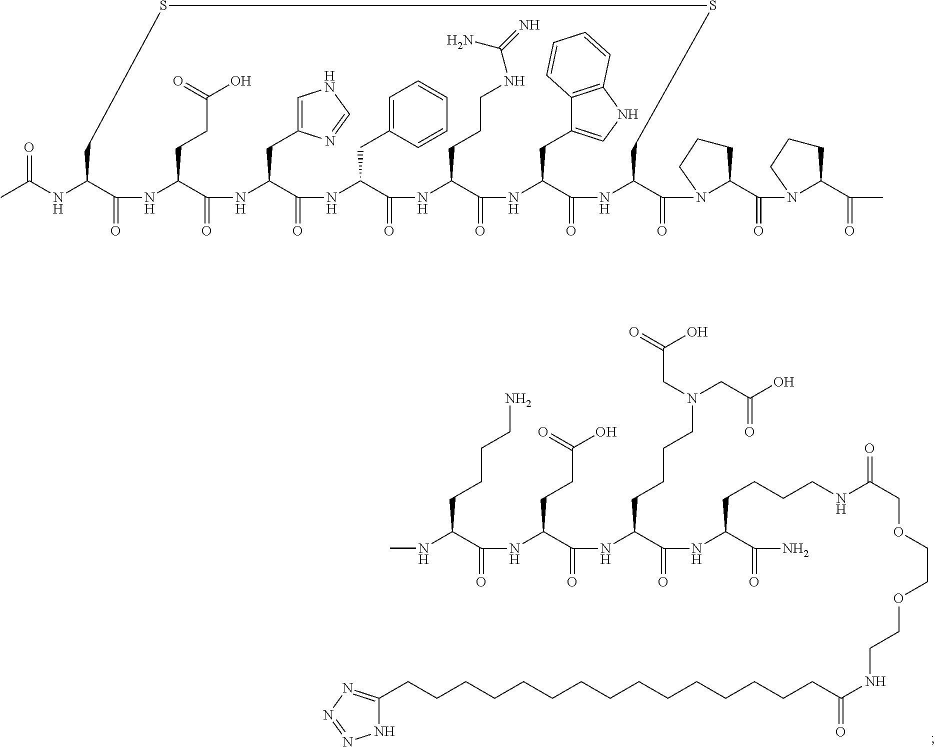 [SMILES: CC(=O)N[C@H]1CSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@H](CCC(=O)O)NC1=O ; CN[C@@H](CCCCN)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[homoCys-His-D-Phe-Arg-Trp-Cys]-Pro-Pro-Lys-Glu-Dap(BCMA)-Lys[(2-{2-[16-(tetrazol-5-yl)hexadecanoylamino]ethoxy}ethoxy)acetyl]-NH₂ 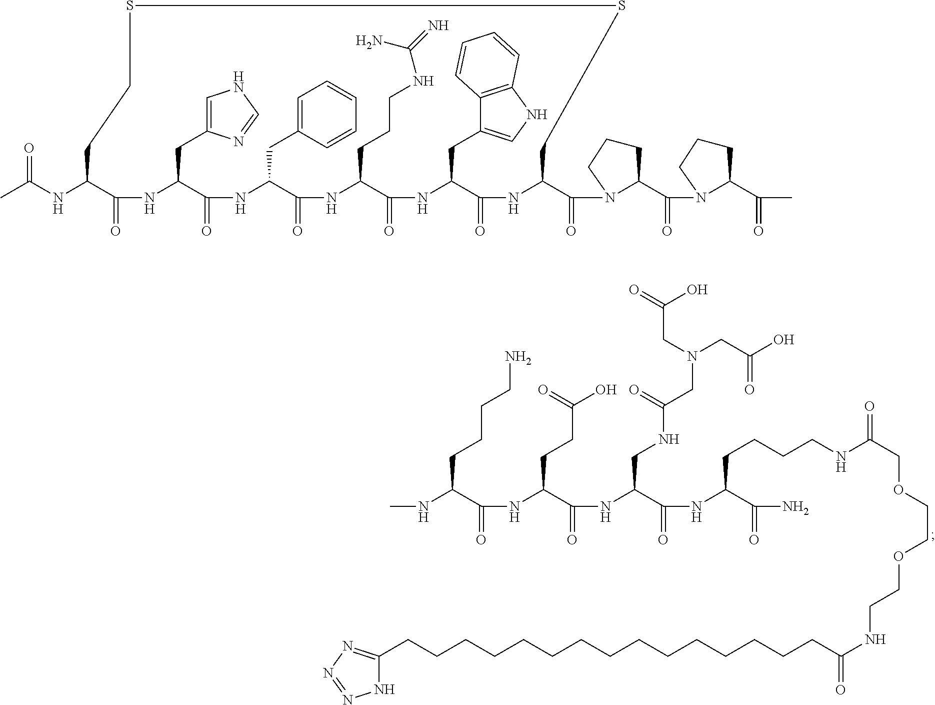 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CCC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[homoCys-His-D-Phe-Arg-Trp-Cys]-Pro-Pro-Lys-Asp-Lys(BCMA)-Lys[(2-{2-[16-(tetrazol-5-yl)hexadecanoylamino]ethoxy}ethoxy)acetyl]-NH₂ 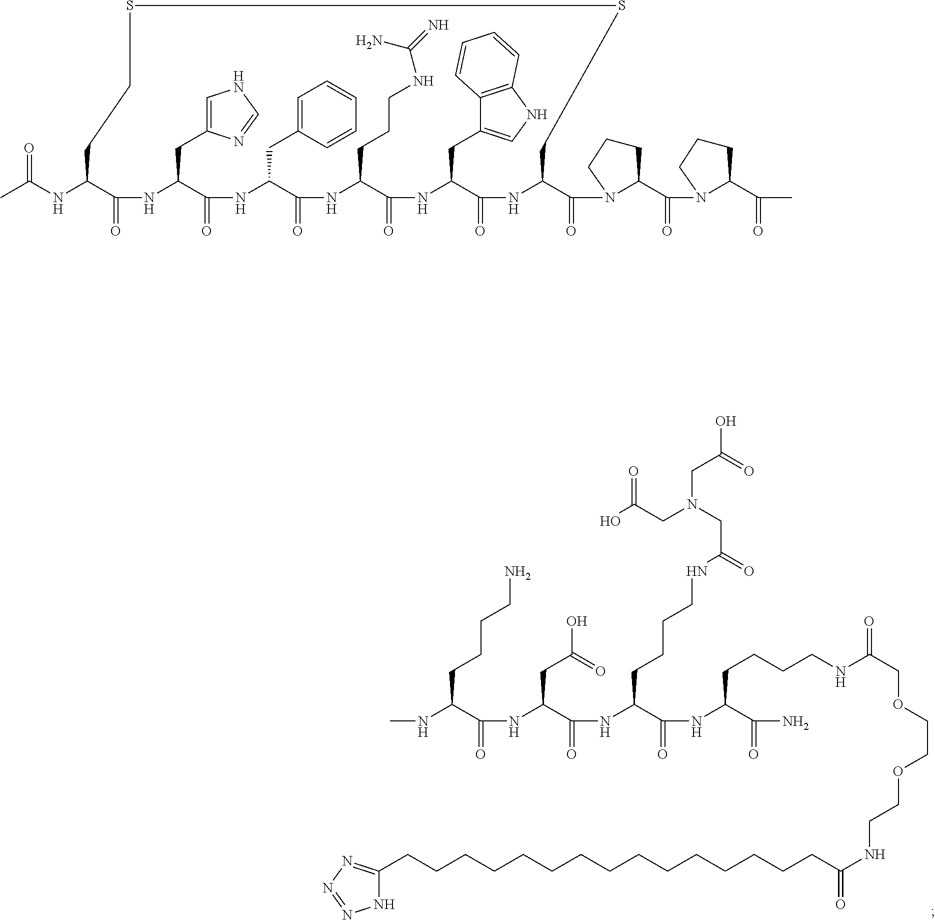 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CCCCN)C(=O)N[C@@H](CC(=O)O)C(=O)N[C@@H](CCCCNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[homoCys-Gln-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂ 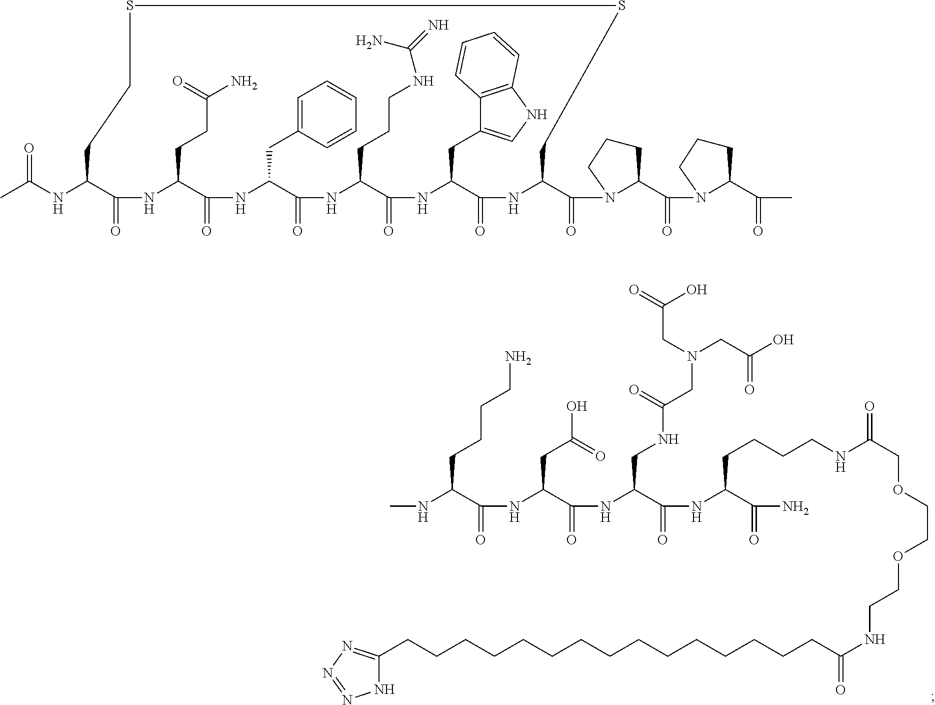 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](CCC(N)=O)NC1=O ; CN[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[homoCys-Hyp-D-Phe-Arg-Trp-Cys]-Pro-Pro-Lys-Asp-Dap(BCMA)-Lys[(2-{2-[16-(tetrazol-5-yl)hexadecanoylamino]ethoxy}ethoxy)acetyl]-NH₂ 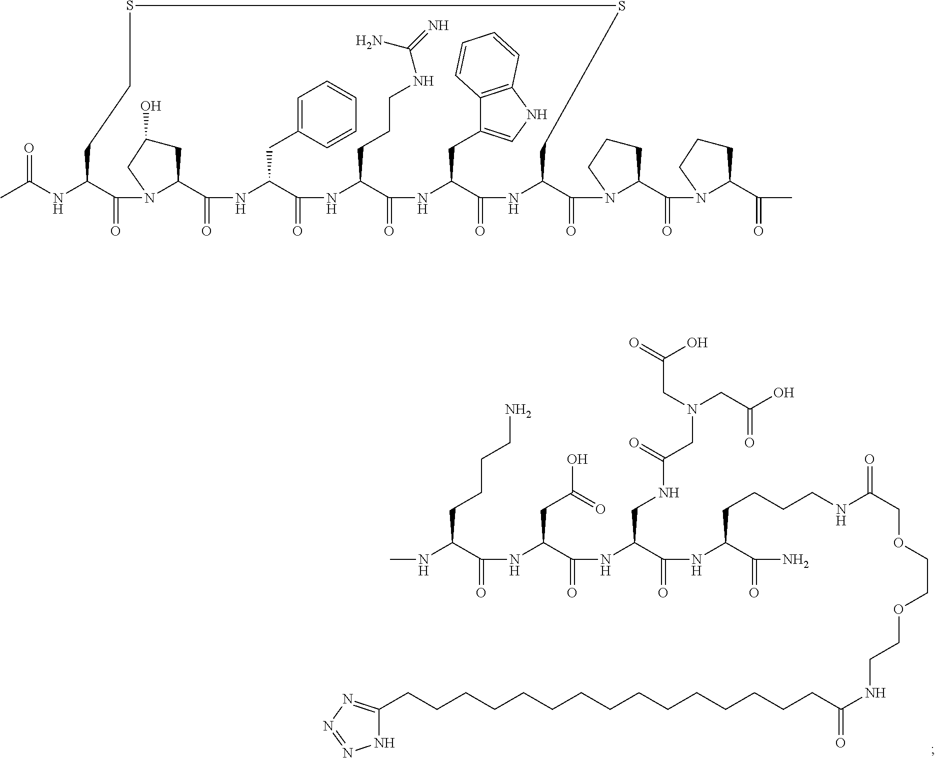 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@@H]2C[C@@H](O)CN2C1=O ; CN[C@@H](CCCCN)C(=O)N[C@@H](CC(=O)O)C(=O)N[C@@H](CNC(=O)CN(CC(=O)O)CC(=O)O)C(=O)N[C@@H](CCCCNC(=O)COCCOCCNC(=O)CCCCCCCCCCCCCCCc1nnn[nH]1)C(N)=O]
Ac-c[Cys-Glu-His-D-Phe-Arg-Trp-Gly-Cys]-Pro-Pro-Lys-Glu-Glu-Lys({2-[2-(15-carboxypentadecanoylamino)ethoxy]ethoxy}acetyl)-NH₂ 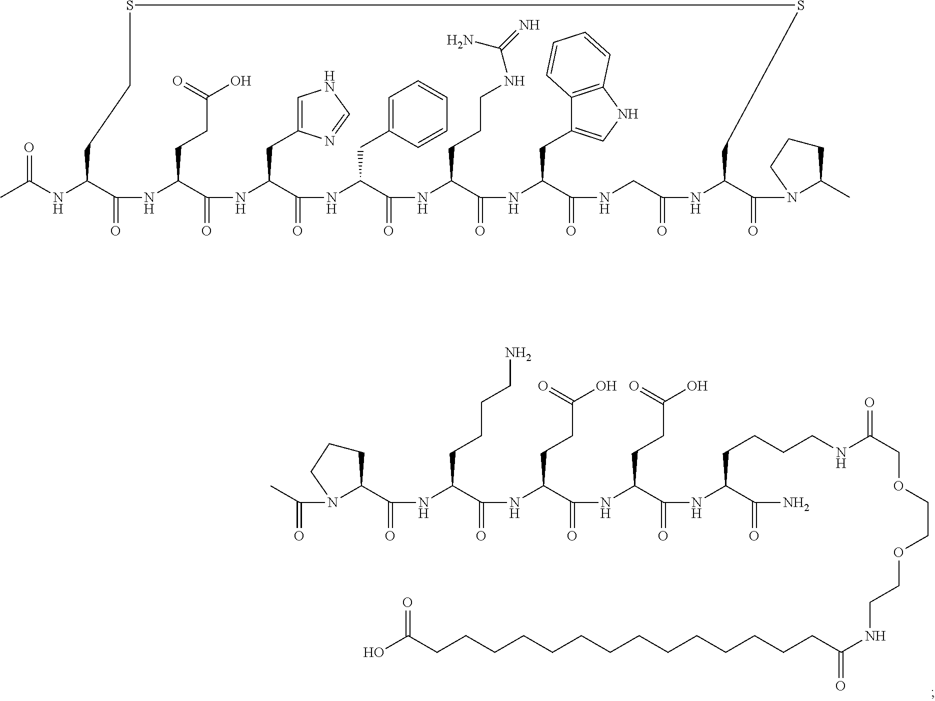
Ac-c[homoCys-His-D-Phe-Arg-Trp-Cys]-Pro-Pro-Lys-Asp-Dap(BCMA)-Lys({2-[2-(13-carboxytridecanoylamino)ethoxy]ethoxy}acetyl)-NH₂ 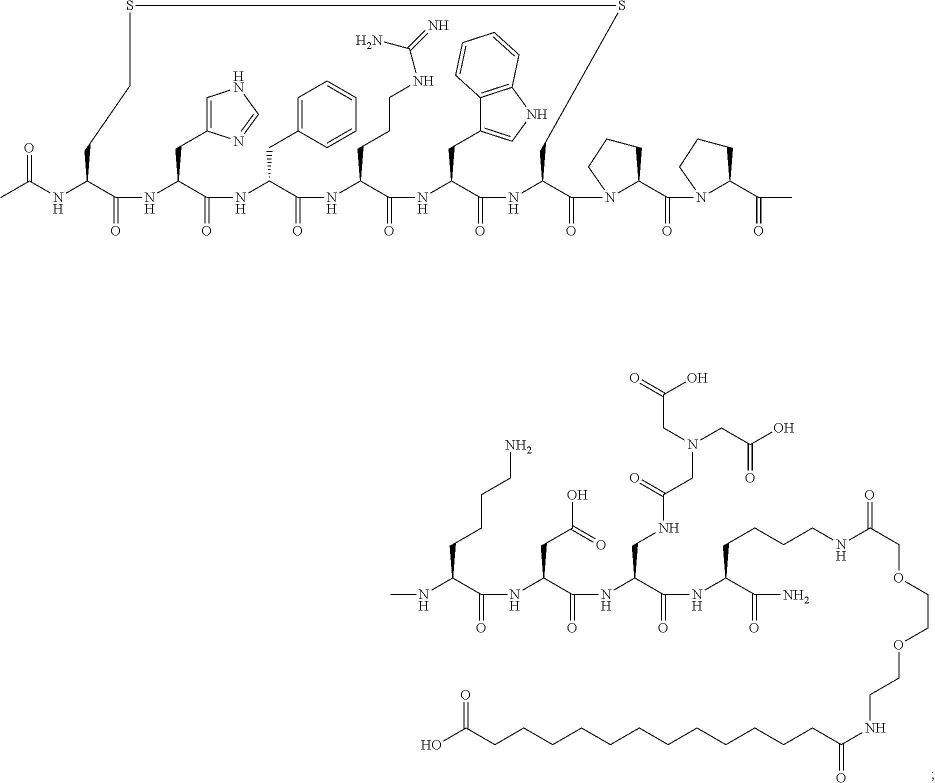
and
Ac-c[homoCys-His-D-Phe-Arg-Trp-Cys]-Pro-Pro-Ser-Glu-Lys(biscarboxymethyl)-Lys({2-[2(15-carboxypentadecanoylamino)ethoxy]ethoxy}acetyl)-NH₂

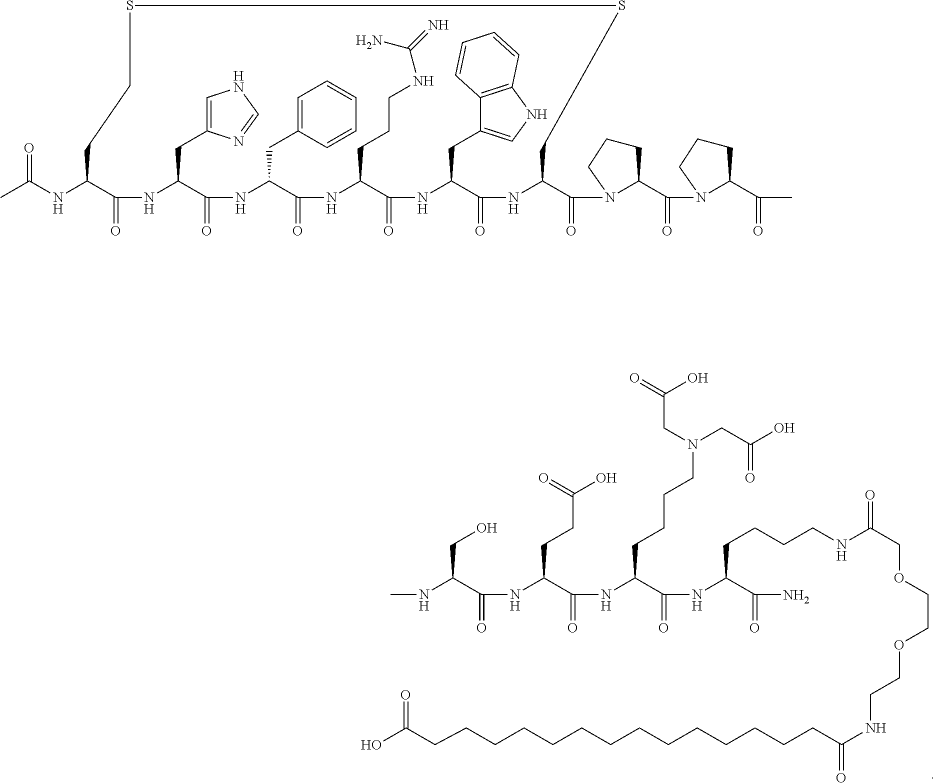 [SMILES: CC(=O)N[C@H]1CCSSC[C@@H](C(=O)N2CCC[C@H]2C(=O)N2CCC[C@H]2C(C)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O ; CN[C@@H](CO)C(=O)N[C@@H](CCC(=O)O)C(=O)N[C@@H](CCCCN(CC(=O)O)CC(=O)O)C(=O)N[C@@H](CCCCNC(=O)COCCOCCNC(=O)CCCCCCCCCCCCCCC(=O)O)C(N)=O]

3. A method of delaying the progression from IGT to type 2 diabetes, comprising administering to a patient in need thereof an effective amount of a compound according to claim 1, optionally in combination with one or more additional therapeutically active compounds.

4. A method of treating obesity or preventing overweight, comprising administering to a patient in need thereof an effective amount of a compound according to claim 1, optionally in combination with one or more additional therapeutically active compounds.

5. A method of regulating appetite, comprising administering to a patient in need thereof an effective amount of a compound according to claim 1, optionally in combination with one or more additional therapeutically active compounds.

6. A method of inducing satiety, comprising administering to a patient in need thereof an effective amount of a compound according to claim 1, optionally in combination with one or more additional therapeutically active compounds.

7. A method of preventing weight gain after successfully having lost weight, comprising administering to a patient in need thereof an effective amount of a compound according to claim 1, optionally in combination with one or more additional therapeutically active compounds.

8. A method of treating a disease or state related to overweight or obesity, comprising administering to a patient in need thereof an effective amount of a compound according to claim 1, optionally in combination with one or more additional therapeutically active compounds.

9. A method of treating bulimia, comprising administering to a patient in need thereof an effective amount of a compound according to claim 1, optionally in combination with one or more additional therapeutically active compounds.

10. A method of treating a disease or state selected from atherosclerosis, hypertension, type 2 diabetes, impaired glucose tolerance (IGT), dyslipidemia, coronary heart dis-ease, gallbladder disease, gall stone, osteoarthritis, cancer, sexual dysfunction and risk of premature death, comprising administering to a patient in need thereof an effective amount of a compound according to claim 1, optionally in combination with one or more additional therapeutically active compounds.

11. A method of treating, in an obese patient, a disease or state selected from atherosclerosis, hypertension, type 2 diabetes, impaired glucose tolerance (IGT), dyslipidemia, coronary heart disease, gallbladder disease, gall stone, osteoarthritis, cancer, sexual dysfunction, hypothalamic amenorrhea or risk of premature death, neuronal protection, effect in ischemic heart disease or anti-inflammatory effects comprising administering to an obese patient in need thereof an effective amount of a compound according to claim 1, optionally in combination with one or more additional therapeutically active compounds.

12. A method according to claim 3, wherein said additional therapeutically active compound is selected from antidiabetic agents, antihyperlipidemic agents, antiobesity agents, antihypertensive agents and agents for the treatment of complications resulting from, or associated with, diabetes.

13. A pharmaceutical composition comprising a compound according to claim 1 and one or more excipients.

14. A compound according to claim 1 for use in therapy.

15. The use of a compound according to claim 1 in the manufacture of a medicament for delaying the progression from impaired glucose tolerance (IGT) to type 2 diabetes; delaying the progression from type 2 diabetes to insulin-requiring diabetes; treating obesity or preventing overweight; regulating appetite; inducing satiety; preventing weight regain after successful weight loss; increasing energy expenditure; treating a disease or state related to overweight or obesity; treating bulimia; treating binge-eating; treating atherosclerosis, hypertension, type 2 diabetes, IGT, dyslipidemia, coronary heart disease, gallbladder disease, gall stone, osteoarthritis, cancer, sexual dysfunction, hypothalamic amenorrhea or risk of premature death; or treating, in an obese patient, a disease or state selected from atherosclerosis, hypertension, type 2 diabetes, IGT, dyspilidemia, coronary heart disease, gallbladder disease, gall stone, osteoarthritis, cancer, sexual dysfunction, hypothalamic amenorrhea or risk of premature death; for providing neuronal protection, for having an effect on ischemic heart disease or anti-inflammatory effects and for the treatment of autoimmune diseases, e.g. multiple sclerosis.
